(19) United States
(12) Patent Application Publication
(10) Pub. No.: US 2025/0066369 A1
(43) Pub. Date: Feb. 27, 2025

(54) BICYCLIC PHTHALAZIN-1(2H)-ONE DERIVATIVES AND RELATED USES

(71) Applicant: NodThera Limited, Little Chesterford (GB)

(72) Inventors: Mark G. BOCK, Boston, MA (US); David HARRISON, Little Chesterford (GB); Jane E. SCANLON, Little Cheseterford (GB)

(21) Appl. No.: 18/726,919

(22) PCT Filed: Jan. 6, 2023
(86) PCT No.: PCT/US2023/010314

§ 371 Date: Jul. 5, 2024

Related U.S. Application Data
(60) Provisional application No. 63/297,444, filed on Jan. 7, 2022.

(51) Int. Cl. C07D 487/04; A61K 31/5025; A61K 31/506; C07D 471/04; C07D 491/048; C07D 495/04; C07D 519/00

(52) U.S. Cl. CPC C07D 487/04; A61K 31/5025; A61K 31/506; C07D 471/04; C07D 491/048; C07D 495/04; C07D 519/00

(57) ABSTRACT

The present disclosure relates to compounds of Formula (III):

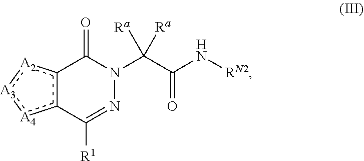

and to their pharmaceutically acceptable salts, pharmaceutical compositions, methods of use, and methods for their preparation. The compounds disclosed herein are useful for inhibiting the maturation of cytokines of the IL-1 family by inhibiting inflammasomes and may be used in the treatment of disorders in which inflammasome activity is implicated, such as inflammatory, autoinflammatory and autoimmune diseases and cancers.

### RELATED APPLICATIONS

This application claims priority to U.S. Provisional Application No. 63/297,444, filed Jan. 7, 2022, the entire contents of which is incorporated herein by reference.

### BACKGROUND

Autoimmune diseases are associated with the overproduction of proinflammatory factors. One of them is interleukin-1 (IL-1), produced by activated macrophages, monocytes, fibroblasts, and other components of the innate immune system like dendritic cells. IL-1 is involved in a variety of cellular activities, including cell proliferation, differentiation and apoptosis (Masters, S. L., et. al., Annu. Rev. Immunol. 2009. 27:621-68).

In humans, 22 NLR proteins are divided into four NLR subfamilies according to their N-terminal domains. NLRA contains a CARD-AT domain, NLRB (NAIP) contains a BIR domain, NLRC (including NOD1 and NOD2) contains a CARD domain, and NLRP contains a pyrin domain. Multiple NLR family members are associated with inflammasome formation.

Although inflammasome activation appears to have evolved as an important component of host immunity to pathogens, the NLRP3 inflammasome is unique in its ability activate in response to endogenous sterile danger signals. Many such sterile signals have been elucidated, and their formation is associated with specific disease states. For example, uric acid crystals found in gout patients are effective triggers of NLRP3 activation. Similarly, cholesterol crystals found in atherosclerotic patients can also promote NLRP3 activation. Recognition of the role of sterile danger signals as NLRP3 activators led to IL-1 and IL-18 being implicated in a diverse range of pathophysiological indications including metabolic, physiologic, inflammatory, hematologic and immunologic disorders.

The disclosure arises from a need to provide further compounds for the specific modulation of NLRP3-dependent cellular processes. In particular, compounds with improved physicochemical, pharmacological and pharmaceutical properties to existing compounds are desirable.

### SUMMARY

In some aspects, the present disclosure relates to a compound of Formula (III):

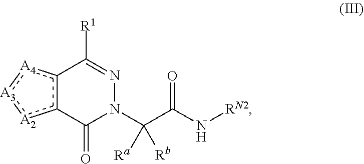

or a prodrug, solvate, or pharmaceutically acceptable salt thereof, wherein:
each [symbol] is independently a single bond or double bond as valency permits;
A₂ is CR², N, NR^{2a}, O, or S, as valency allows;
A₃ is CR², N, NR^{2a}, O, or S, as valency allows;
A₄ is CR², N, NR^{2a}, O, or S, as valency allows,
wherein at least one of A₂, A₃, or A₄ is N, NR^{2a}, O, or S, provided that when A₂ is S, A₄ is CR², NR^{2a}, O, or S;
R¹ is H, —N(C₁-C₆ alkyl)₂, C₁-C₆ alkyl, C₂-C₆ alkenyl, or C₃-C₁₂ cycloalkyl, wherein the —N(C₁-C₆ alkyl)₂, C₁-C₆ alkyl, C₂-C₆ alkenyl, or C₃-C₁₂ cycloalkyl is optionally substituted with one or more R^{1S};
each R^{1S} independently is halogen, cyano, —OH, or C₁-C₆ alkyl;
each R² independently is H, halogen, cyano, —OH, —NH₂, —NO₂, —C(═O)NH₂, C₁-C₆ alkyl, C₂-C₆ alkenyl, C₂-C₆ alkynyl, —O(C₁-C₆ alkyl), —NH(C₁-C₆ alkyl), —N(C₁-C₆ alkyl)₂, C₃-C₁₂ cycloalkyl, 3- to 12-membered heterocycloalkyl, C₆-C₁₀ aryl, or 5- to 10-membered heteroaryl, wherein the C₁-C₆ alkyl, C₂-C₆ alkenyl, C₂-C₆ alkynyl, —O(C₁-C₆ alkyl), —NH(C₁-C₆ alkyl), —N(C₁-C₆ alkyl)₂, C₃-C₁₂ cycloalkyl, 3- to 12-membered heterocycloalkyl, C₆-C₁₀ aryl, or 5- to 10-membered heteroaryl is optionally substituted with one or more R^{2S},
or two R² together with the atoms to which they are attached form a C₃-C₁₂ cycloalkyl or 3- to 12-membered heterocycloalkyl, wherein the C₃-C₁₂ cycloalkyl or 3- to 12-membered heterocycloalkyl is optionally substituted with one or more R^{2S};
each R^{2S} independently is halogen, —OH, —O(C₁-C₆ alkyl), —NH₂, —NH(C₁-C₆ alkyl), —N(C₁-C₆ alkyl)₂, or C₃-C₁₂ cycloalkyl;
each R^{2a} independently is H, C₁-C₆ alkyl, C₂-C₆ alkenyl, C₂-C₆ alkynyl, C₁-C₆ haloalkyl, —(CH₂)₀₋₃—(C₃-C₁₂ cycloalkyl), or —(CH₂)₀₋₃-(3- to 12-membered heterocycloalkyl);
each R^{a} independently is H or C₁-C₆ alkyl; or two R^{a}, together with the atom they attach to, form C₃-C₁₂ cycloalkyl;
R^{N2} is C₁-C₆ alkyl, C₂-C₆ alkenyl, C₂-C₆ alkynyl, —O—(C₁-C₆ alkyl), —O—(C₂-C₆ alkenyl), —O—(C₂-C₆ alkynyl), —NH—(C₁-C₆ alkyl), —NH—(C₂-C₆ alkenyl), —NH—(C₂-C₆ alkynyl), C₃-C₁₂ cycloalkyl, 3- to 12-membered heterocycloalkyl, C₆-C₁₀ aryl, 5- to 10-membered heteroaryl, —(C₁-C₆ alkyl)-(C₃-C₁₂ cycloalkyl), —(C₁-C₆ alkyl)-(3- to 12-membered heterocycloalkyl), —(C₁-C₆ alkyl)-(C₆-C₁₀ aryl), or —(C₁-C₆ alkyl)-(5- to 10-membered heteroaryl); wherein the C₁-C₆ alkyl, C₂-C₆ alkenyl, C₂-C₆ alkynyl, —O—(C₁-C₆ alkyl), —O—(C₂-C₆ alkenyl), —O—(C₂-C₆ alkynyl), —NH—(C₁-C₆ alkyl), —NH—(C₂-C₆ alkenyl), —NH—(C₂-C₆ alkynyl), C₃-C₁₂ cycloalkyl, 3- to 12-membered heterocycloalkyl, C₆-C₁₀ aryl, 5- to 10-membered heteroaryl, —(C₁-C₆ alkyl)-(C₃-C₁₂ cycloalkyl), —(C₁-C₆ alkyl)-(3- to 12-membered heterocycloalkyl), —(C₁-C₆ alkyl)-(C₆-C₁₀ aryl), or —(C₁-C₆ alkyl)-(5- to 10-membered heteroaryl) is optionally substituted with one or more R^{N2a};
each R^{N2a} independently is oxo, halogen, cyano, —OH, —NH₂, —C(═O)H, —C(═O)OH, C₁-C₆ alkyl, C₂-C₆ alkenyl, C₂-C₆ alkynyl, —O(C₁-C₆ alkyl), —NH(C₁-C₆ alkyl), —N(C₁-C₆ alkyl)₂, —C(═O)(C₁-C₆ alkyl), —C(═O)O(C₁-C₆ alkyl), —NHC(═O)O(C₁-C₆ alkyl), —S(═O)₂(C₁-C₆ alkyl), —S(═O)₂N(C₁-C₆ alkyl)₂, C₃-C₁₂ cycloalkyl, 3- to 12-membered heterocycloalkyl, C₆-C₁₀ aryl, 5- to 10-membered heteroaryl, —(C₁-C₆ alkyl)-(C₃-C₁₂ cycloalkyl), —(C₁-C₆ alkyl)-(3- to 12-membered heterocycloalkyl), —(C₁-C₆ alkyl)-(C₆-C₁₀ aryl), or —(C₁-C₆ alkyl)-(5- to 10-membered heteroaryl); wherein the C₁-C₆ alkyl, C₂-C₆ alkenyl, C₂-C₆ alkynyl, —O(C₁-C₆ alkyl), —NH(C₁-C₆ alkyl), —N(C₁-C₆ alkyl)₂, —C(═O)(C₁-C₆ alkyl), —C(═O)O(C₁-C₆ alkyl), —NHC(═O)O(C₁-C₆ alkyl), —S(═O)₂(C₁-C₆ alkyl), —S(═O)₂N(C₁-C₆ alkyl)₂, C₃-C₁₂ cycloalkyl, 3- to 12-membered heterocycloalkyl, C₆-C₁₀ aryl, 5- to 10-membered heteroaryl, —(C₁-C₆ alkyl)-(C₃-C₁₂ cycloalkyl), —(C₁-C₆ alkyl)-(3- to 12-membered heterocycloalkyl), —(C₁-C₆ alkyl)-(C₆-C₁₀ aryl), or —(C₁-C₆ alkyl)-(5- to 10-membered heteroaryl) is optionally substituted with one or more R^{N2ab}; and
each R^{N2ab} independently is oxo, halogen, cyano, —OH, —NH₂, —C(═O)H, —C(═O)OH, —O(C₁-C₆ alkyl), —NH(C₁-C₆ alkyl), —N(C₁-C₆ alkyl)₂, —C(═O)(C₁-C₆ alkyl), —C(═O)O(C₁-C₆ alkyl), —NHC(═O)O(C₁-C₆ alkyl), —S(═O)₂(C₁-C₆ alkyl), or —S(═O)₂N(C₁-C₆ alkyl)₂.

In some aspects, the present disclosure provides a compound obtainable by, or obtained by, a method for preparing a compound as described herein.

In some embodiments, the present disclosure provides a pharmaceutical composition comprising a compound described herein and one or more pharmaceutically acceptable carriers or excipients.

In some aspects, the present disclosure provides an intermediate as described herein, being suitable for use in a method for preparing a compound as described herein.

In some aspects, the present disclosure provides a method of inhibiting inflammasome (e.g., the NLRP3 inflammasome) activity (e.g., in vitro or in vivo), comprising contacting a cell with an effective amount of a compound of the present disclosure.

In some aspects, the present disclosure provides a method of treating or preventing a disease or disorder disclosed herein in a subject in need thereof, comprising administering to the subject a compound of the present disclosure or a pharmaceutical composition of the present disclosure.

In some aspects, the present disclosure provides a method of treating or preventing a disease or disorder disclosed herein in a subject in need thereof, comprising administering to the subject a therapeutically effective amount of a compound of the present disclosure or a pharmaceutical composition of the present disclosure.

In some aspects, the present disclosure provides a compound of the present disclosure for use in inhibiting inflammasome (e.g., the NLRP3 inflammasome) activity (e.g., in vitro or in vivo).

In some aspects, the present disclosure provides a compound of the present disclosure or a pharmaceutically acceptable salt thereof for use in treating or preventing a disease or disorder disclosed herein.

In some aspects, the present disclosure provides use of a compound of the present disclosure in the manufacture of a medicament for inhibiting inflammasome (e.g., the NLRP3 inflammasome) activity (e.g., in vitro or in vivo).

In some aspects, the present disclosure provides use of a compound of the present disclosure in the manufacture of a medicament for treating or preventing a disease or disorder disclosed herein.

In some aspects, the present disclosure provides a method of preparing a compound of the present disclosure.

In some aspects, the present disclosure provides a method of a compound, comprising one or more steps described herein.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods, and examples are illustrative only and are not intended to be limiting. In the case of conflict between the chemical structures and names of the compounds disclosed herein, the chemical structures will control.

Other features and advantages of the disclosure will be apparent from the following detailed description and claims.

### DETAILED DESCRIPTION

Autoimmune diseases are associated with the overproduction of proinflammatory factors. One of them is interleukin-1 (IL-1), produced by activated macrophages, monocytes, fibroblasts, and other components of the innate immune system like dendritic cells, involved in a variety of cellular activities, including cell proliferation, differentiation and apoptosis (Masters, S. L. et al., Annu. Rev. Immunol. 2009. 27:621-68).

Autoimmune diseases are associated with the overproduction of proinflammatory factors. One of them is interleukin-1 (IL-1), produced by activated macrophages, monocytes, fibroblasts, and other components of the innate immune system like dendritic cells, involved in a variety of cellular activities, including cell proliferation, differentiation, and apoptosis (Masters, S. L., et al. Annu. Rev. Immunol. 2009. 27:621-68).

Cytokines from the IL-1 family are highly active and, as important mediators of inflammation, primarily associated with acute and chronic inflammation (Sims, J. et al., Nature Reviews Immunology 10, 89-102 (February 2010)). The overproduction of IL-1 is considered to be a mediator of some autoimmune and autoinflammatory diseases. Autoinflammatory diseases are characterised by recurrent and unprovoked inflammation in the absence of autoantibodies, infection, or antigen-specific T lymphocytes.

Proinflammatory cytokines of the IL-1 superfamily include IL-1α, IL-1β, IL-18, and IL-36α, β, λ and are produced in response to pathogens and other cellular stressors as part of a host innate immune response. Unlike many other secreted cytokines, which are processed and released via the standard cellular secretory apparatus consisting of the endoplasmic reticulum and Golgi apparatus, IL-1 family members lack leader sequences required for endoplasmic reticulum entry and thus are retained intracellularly following translation. In addition, IL-1β, IL-18, and IL-36α, β, λ are synthesised as procytokines that require proteolytic activation to become optimal ligands for binding to their cognate receptors on target cells.

In the case of IL-1α, IL-1β and IL-18, it is now appreciated that a multimeric protein complex known as an inflammasome is responsible for activating the proforms of IL-1β and IL-18 and for release of these cytokines extracellularly. An inflammasome complex typically consists of a sensor molecule, such as an NLR (Nucleotide-Oligerimisation Domain (NOD)-like receptor), an adaptor molecule ASC (Apoptosis-associated speck-like protein containing a CARD (Caspase Recruitment Domain)) and procaspase-1. In response to a variety of “danger signals”, including pathogen-associated molecule patterns (PAMPs) and danger associated molecular patterns (DAMPs), subunits of an inflammasome oligomerise to form a supramolecular structure within the cell. PAMPs include molecules such as peptidoglycan, viral DNA or RNA and bacterial DNA or RNA. DAMPs, on the other hand, consist of a wide range of endogenous or exogenous sterile triggers including monosodium urate crystals, silica, alum, asbestos, fatty acids, ceramides, cholesterol crystals and aggregates of beta-amyloid peptide. Assembly of an inflammasome platform facilitates autocatalysis of procaspase-1 yielding a highly active cysteine protease responsible for activation and release of pro-IL-1β and pro-IL-18. Thus, release of these highly inflammatory cytokines is achieved only in response to inflammasome sensors detecting and responding to specific molecular danger signals.

In humans, 22 NLR proteins are divided into four NLR subfamilies according to their N-terminal domains. NLRA contains a CARD-AT domain, NLRB (NAIP) contains a BIR domain, NLRC (including NOD1 and NOD2) contains a CARD domain, and NLRP contains a pyrin domain. Multiple NLR family members are associated with inflammasome formation including NLRP1, NLRP3, NLRP6, NLRP7, NLRP12 and NLRC4 (IPAF).

Two other structurally distinct inflammasome structures containing a PYHIN domain (pyrin and HIN domain containing protein) namely Absent in Melanoma 2 (AIM2) and IFNλ-inducible protein 16 (IFI16) (Latz et al., Nat Rev Immunol 2013 13(6) 397-311) serve as intracellular DNA sensors. Pyrin (encoded by the MEFV gene) represents another type of inflammasome platform associated with proIL-1β activation (Chae et al., Immunity 34, 755-768, 2011).

Requiring assembly of an inflammasome platform to achieve activation and release of IL-1β and IL-18 from monocytes and macrophages ensures their production is carefully orchestrated via a 2-step process. First, the cell must encounter a priming ligand (such as the TLR4 receptor ligand LPS, or an inflammatory cytokine such as TNFα) which leads to NFkB dependent transcription of NLRP3, pro-IL-1β and pro-IL-18. The newly translated procytokines remain intracellular and inactive unless producing cells encounter a second signal leading to activation of an inflammasome scaffold and maturation of procaspase-1.

In addition to proteolytic activation of pro-IL-1β and pro-IL-18, active caspase-1 also triggers a form of inflammatory cell death known as pyroptosis through cleavage of gasdermin-D. Pyroptosis allows the mature forms of IL-1β and IL-18 to be externalised along with release of alarmin molecules (compounds that promote inflammation and activate innate and adaptive immunity) such as high mobility group box 1 protein (HMGB1), IL-33, and IL-1α.

Although inflammasome activation appears to have evolved as an important component of host immunity to pathogens, the NLRP3 inflammasome is unique in its ability activate in response to endogenous and exogenous sterile danger signals. Many such sterile signals have been elucidated, and their formation is associated with specific disease states. For example, uric acid crystals found in gout patients are effective triggers of NLRP3 activation. Similarly, cholesterol crystals found in atherosclerotic patients can also promote NLRP3 activation. Recognition of the role of sterile danger signals as NLRP3 activators led to IL-1p and IL-18 being implicated in a diverse range of pathophysiological indications including metabolic, physiologic, inflammatory, hematologic and immunologic disorders.

A link to human disease is best exemplified by discovery that mutations in the NLRP3 gene which lead to gain-of-function confer a range of autoinflammatory conditions collectively known as cryopyrin-associated periodic syndromes (CAPS) including familial cold autoinflammatory syndrome (FCAS), Muckle-Wells syndrome (MWS) and Neonatal onset multisystem inflammatory disease (NOMID) (Hoffman et al., Nat. Genet. 29(3) (2001) 301-305). Likewise, sterile mediator-induced activation of NLRP3 has been implicated in a wide range of disorders including joint degeneration (gout, rheumatoid arthritis, osteoarthritis), cardiometabolic (type 2 diabetes, atherosclerosis, hypertension), Central Nervous System (Alzheimer's Disease, Parkinson's disease, multiple sclerosis), gastrointestinal (Crohn's disease, ulcerative colitis), lung (chronic obstructive pulmonary disease (COPD), asthma, idiopathic pulmonary fibrosis) and liver (fibrosis, non-alcoholic fatty liver disease, non-alcoholic steatohepatitis (NASH)). It is further believed that NLRP3 activation promotes kidney inflammation and thus contributes to chronic kidney disease (CKD).

Current treatment options for diseases where IL-1 is implicated as a contributor to pathogenesis include the IL-1 receptor antagonist anakinra, an Fc-containing fusion construct of the extracellular domains of the IL-1 receptor and IL-1 receptor accessory protein (rilonacept) and the anti-IL-1β monoclonal antibody canakinumab. For example, canakinumab is licensed for CAPS, Tumor Necrosis Factor Receptor Associated Periodic Syndrome (TRAPS), Hyperimmunoglobulin D Syndrome (HIDS)/Mevalonate Kinase Deficiency (MKD), Familial Mediterranean Fever (FMF) and gout.

Some small molecules have been reported to inhibit function of the NLRP3 inflammasome. Glyburide, for example, is a specific inhibitor of NLRP3 activation, albeit at micromolar concentrations which are unlikely attainable in vivo. Non-specific agents such as parthenolide, Bay 11-7082, and 3,4-methylenedioxy-β-nitrostyrene are reported to impair NLRP3 activation but are expected to possess limited therapeutic utility due to their sharing of a common structural feature consisting of an olefin activated by substitution with an electron withdrawing group; this can lead to undesirable formation of covalent adducts with protein-bearing thiol groups. A number of natural products, for example β-hydroxybutyrate, sulforaphane, quercetin, and salvianolic acid, also are reported to suppress NLRP3 activation. Likewise, numerous effectors/modulators of other molecular targets have been reported to impair NLRP3 activation including agonists of the G-protein coupled receptor TGR5, an inhibitor of sodium-glucose co-transport epigliflozin, the dopamine receptor antagonist A-68930, the serotonin reuptake inhibitor fluoxetine, fenamate non-steroidal anti-inflammatory drugs, and the β-adrenergic receptor blocker nebivolol. Utility of these molecules as therapeutics for the chronic treatment of NLRP3-dependent inflammatory disorders remains to be established.

The disclosure relates to compounds useful for the specific modulation of NLRP3-dependent cellular processes. In particular, compounds with improved physicochemical, pharmacological and pharmaceutical properties to existing NLRP3-modulating compounds are desired.

### Compounds of the Present Disclosure

In some aspects, the present disclosure relates to a compound of Formula (III):

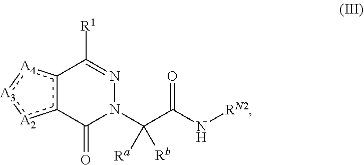

or a prodrug, solvate, or pharmaceutically acceptable salt thereof, wherein:
each [symbol] is independently a single bond or double bond as valency permits;
A₂ is CR², N, NR^{2a}, O, or S, as valency allows;
A₃ is CR², N, NR^{2a}, O, or S, as valency allows;
A₄ is CR², N, NR^{2a}, O, or S, as valency allows,
wherein at least one of A₂, A₃, or A₄ is N, NR^{2a}, O, or S, provided that when A₂ is S, A₄ is CR², NR^{2a}, O, or S;
R¹ is H, —N(C₁-C₆ alkyl)₂, C₁-C₆ alkyl, C₂-C₆ alkenyl, or C₃-C₁₂ cycloalkyl, wherein the —N(C₁-C₆ alkyl)₂, C₁-C₆ alkyl, C₂-C₆ alkenyl, or C₃-C₁₂ cycloalkyl is optionally substituted with one or more R^{1S};
each R^{1S} independently is halogen, cyano, —OH, or C₁-C₆ alkyl;
each R² independently is H, halogen, cyano, —OH, —NH₂, —NO₂, —C(═O)NH₂, C₁-C₆ alkyl, C₂-C₆ alkenyl, C₂-C₆ alkynyl, —O(C₁-C₆ alkyl), —NH(C₁-C₆ alkyl), —N(C₁-C₆ alkyl)₂, C₃-C₁₂ cycloalkyl, 3- to 12-membered heterocycloalkyl, C₆-C₁₀ aryl, or 5- to 10-membered heteroaryl, wherein the C₁-C₆ alkyl, C₂-C₆ alkenyl, C₂-C₆ alkynyl, —O(C₁-C₆ alkyl), —NH(C₁-C₆ alkyl), —N(C₁-C₆ alkyl)₂, C₃-C₁₂ cycloalkyl, 3- to 12-membered heterocycloalkyl, C₆-C₁₀ aryl, or 5- to 10-membered heteroaryl is optionally substituted with one or more R^{2S},
or two R² together with the atoms to which they are attached form a C₃-C₁₂ cycloalkyl or 3- to 12-membered heterocycloalkyl, wherein the C₃-C₁₂ cycloalkyl or 3- to 12-membered heterocycloalkyl is optionally substituted with one or more R^{2S};
each R^{2S} independently is halogen, —OH, —O(C₁-C₆ alkyl), —NH₂, —NH(C₁-C₆ alkyl), —N(C₁-C₆ alkyl)₂, or C₃-C₁₂ cycloalkyl;
each R^{2a} independently is H, C₁-C₆ alkyl, C₂-C₆ alkenyl, C₂-C₆ alkynyl, C₁-C₆ haloalkyl, —(CH₂)₀₋₃—(C₃-C₁₂ cycloalkyl), or —(CH₂)₀₋₃-(3- to 12-membered heterocycloalkyl);
each R^{a} independently is H or C₁-C₆ alkyl; or two R^{a}, together with the atom they attach to, form C₃-C₁₂ cycloalkyl;
R^{N2} is C₁-C₆ alkyl, C₂-C₆ alkenyl, C₂-C₆ alkynyl, —O—(C₁-C₆ alkyl), —O—(C₂-C₆ alkenyl), —O—(C₂-C₆ alkynyl), —NH—(C₁-C₆ alkyl), —NH—(C₂-C₆ alkenyl), —NH—(C₂-C₆ alkynyl), C₃-C₁₂ cycloalkyl, 3- to 12-membered heterocycloalkyl, C₆-C₁₀ aryl, 5- to 10-membered heteroaryl, —(C₁-C₆ alkyl)-(C₃-C₁₂ cycloalkyl), —(C₁-C₆ alkyl)-(3- to 12-membered heterocycloalkyl), —(C₁-C₆ alkyl)-(C₆-C₁₀ aryl), or —(C₁-C₆ alkyl)-(5- to 10-membered heteroaryl); wherein the C₁-C₆ alkyl, C₂-C₆ alkenyl, C₂-C₆ alkynyl, —O—(C₁-C₆ alkyl), —O—(C₂-C₆ alkenyl), —O—(C₂-C₆ alkynyl), —NH—(C₁-C₆ alkyl), —NH—(C₂-C₆ alkenyl), —NH—(C₂-C₆ alkynyl), C₃-C₁₂ cycloalkyl, 3- to 12-membered heterocycloalkyl, C₆-C₁₀ aryl, 5- to 10-membered heteroaryl, —(C₁-C₆ alkyl)-(C₃-C₁₂ cycloalkyl), —(C₁-C₆ alkyl)-(3- to 12-membered heterocycloalkyl), —(C₁-C₆ alkyl)-(C₆-C₁₀ aryl), or —(C₁-C₆ alkyl)-(5- to 10-membered heteroaryl) is optionally substituted with one or more R^{N2a};
each R^{N2a} independently is oxo, halogen, cyano, —OH, —NH₂, —C(═O)H, —C(═O)OH, C₁-C₆ alkyl, C₂-C₆ alkenyl, C₂-C₆ alkynyl, —O(C₁-C₆ alkyl), —NH(C₁-C₆ alkyl), —N(C₁-C₆ alkyl)₂, —C(═O)(C₁-C₆ alkyl), —C(═O)O(C₁-C₆ alkyl), —NHC(═O)O(C₁-C₆ alkyl), —S(═O)₂(C₁-C₆ alkyl), —S(═O)₂N(C₁-C₆ alkyl)₂, C₃-C₁₂ cycloalkyl, 3- to 12-membered heterocycloalkyl, C₆-C₁₀ aryl, 5- to 10-membered heteroaryl, —(C₁-C₆ alkyl)-(C₃-C₁₂ cycloalkyl), —(C₁-C₆ alkyl)-(3- to 12-membered heterocycloalkyl), —(C₁-C₆ alkyl)-(C₆-C₁₀ aryl), or —(C₁-C₆ alkyl)-(5- to 10-membered heteroaryl); wherein the C₁-C₆ alkyl, C₂-C₆ alkenyl, C₂-C₆ alkynyl, —O(C₁-C₆ alkyl), —NH(C₁-C₆ alkyl), —N(C₁-C₆ alkyl)₂, —C(═O)(C₁-C₆ alkyl), —C(═O)O(C₁-C₆ alkyl), —NHC(═O)O(C₁-C₆ alkyl), —S(═O)₂(C₁-C₆ alkyl), —S(═O)₂N(C₁-C₆ alkyl)₂, C₃-C₁₂ cycloalkyl, 3- to 12-membered heterocycloalkyl, C₆-C₁₀ aryl, 5- to 10-membered heteroaryl, —(C₁-C₆ alkyl)-(C₃-C₁₂ cycloalkyl), —(C₁-C₆ alkyl)-(3- to 12-membered heterocycloalkyl), —(C₁-C₆ alkyl)-(C₆-C₁₀ aryl), or —(C₁-C₆ alkyl)-(5- to 10-membered heteroaryl) is optionally substituted with one or more R^{N2ab}; and
each R^{N2ab} independently is oxo, halogen, cyano, —OH, —NH₂, —C(═O)H, —C(═O)OH, —O(C₁-C₆ alkyl), —NH(C₁-C₆ alkyl), —N(C₁-C₆ alkyl)₂, —C(═O)(C₁-C₆ alkyl), —C(═O)O(C₁-C₆ alkyl), —NHC(═O)O(C₁-C₆ alkyl), —S(═O)₂(C₁-C₆ alkyl), or —S(═O)₂N(C₁-C₆ alkyl)₂.

In some aspects, the present disclosure relates to a compound of Formula (I):

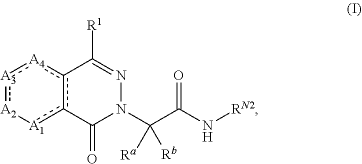

or a prodrug, solvate, or pharmaceutically acceptable salt thereof, wherein:
each [symbol] is independently a single bond or double bond as valency permits;
A₁ is absent, CR², N, NR^{2a}, O, or S, as valency allows;
A₂ is CR², N, NR^{2a}, O, or S, as valency allows;
A₃ is CR², N, NR^{2a}, O, or S, as valency allows;
A₄ is CR², N, NR^{2a}, O, or S, as valency allows,
wherein at least one of A₁, A₂, A₃, or A₄ is N, NR^{2a}, O, or S;
R¹ is H, —N(C₁-C₆ alkyl)₂, C₁-C₆ alkyl, C₂-C₆ alkenyl, or C₃-C₁₂ cycloalkyl, wherein the —N(C₁-C₆ alkyl)₂, C₁-C₆ alkyl, C₂-C₆ alkenyl, or C₃-C₁₂ cycloalkyl is optionally substituted with one or more R^{1S};
each R^{1S} independently is halogen, cyano, —OH, or C₁-C₆ alkyl;
each R² independently is H, halogen, cyano, —OH, —NH₂, —NO₂, —C(═O)NH₂, C₁-C₆ alkyl, C₂-C₆ alkenyl, C₂-C₆ alkynyl, —O(C₁-C₆ alkyl), —NH(C₁-C₆ alkyl), —N(C₁-C₆ alkyl)₂, C₃-C₁₂ cycloalkyl, 3- to 12-membered heterocycloalkyl, C₆-C₁₀ aryl, or 5- to 10-membered heteroaryl, wherein the C₁-C₆ alkyl, C₂-C₆ alkenyl, C₂-C₆ alkynyl, —O(C₁-C₆ alkyl), —NH(C₁-C₆ alkyl), —N(C₁-C₆ alkyl)₂, C₃-C₁₂ cycloalkyl, 3- to 12-membered heterocycloalkyl, C₆-C₁₀ aryl, or 5- to 10-membered heteroaryl is optionally substituted with one or more R^{2S},
or two R² together with the atoms to which they are attached form a C₃-C₁₂ cycloalkyl or 3- to 12-membered heterocycloalkyl, wherein the C₃-C₁₂ cycloalkyl or 3- to 12-membered heterocycloalkyl is optionally substituted with one or more R^{2S};
each R^{2S} independently is halogen, —OH, —O(C₁-C₆ alkyl), —NH₂, —NH(C₁-C₆ alkyl), —N(C₁-C₆ alkyl)₂, or C₃-C₁₂ cycloalkyl;
each R^{2a} independently is H, C₁-C₆ alkyl, C₂-C₆ alkenyl, C₂-C₆ alkynyl, C₁-C₆ haloalkyl, —(CH₂)₀₋₃—(C₃-C₁₂ cycloalkyl), or —(CH₂)₀₋₃-(3- to 12-membered heterocycloalkyl);
each R^{a} independently is H or C₁-C₆ alkyl; or two R^{a}, together with the atom they attach to, form C₃-C₁₂ cycloalkyl;
R^{N1} is H or C₁-C₆ alkyl;
R^{N2} is C₁-C₆ alkyl, C₂-C₆ alkenyl, C₂-C₆ alkynyl, —O—(C₁-C₆ alkyl), —O—(C₂-C₆ alkenyl), —O—(C₂-C₆ alkynyl), —NH—(C₁-C₆ alkyl), —NH—(C₂-C₆ alkenyl), —NH—(C₂-C₆ alkynyl), C₃-C₁₂ cycloalkyl, 3- to 12-membered heterocycloalkyl, C₆-C₁₀ aryl, 5- to 10-membered heteroaryl, —(C₁-C₆ alkyl)-(C₃-C₁₂ cycloalkyl), —(C₁-C₆ alkyl)-(3- to 12-membered heterocycloalkyl), —(C₁-C₆ alkyl)-(C₆-C₁₀ aryl), or —(C₁-C₆ alkyl)-(5- to 10-membered heteroaryl); wherein the C₁-C₆ alkyl, C₂-C₆ alkenyl, C₂-C₆ alkynyl, —O—(C₁-C₆ alkyl), —O—(C₂-C₆ alkenyl), —O—(C₂-C₆ alkynyl), —NH—(C₁-C₆ alkyl), —NH—(C₂-C₆ alkenyl), —NH—(C₂-C₆ alkynyl), C₃-C₁₂ cycloalkyl, 3- to 12-membered heterocycloalkyl, C₆-C₁₀ aryl, 5- to 10-membered heteroaryl, —(C₁-C₆ alkyl)-(C₃-C₁₂ cycloalkyl), —(C₁-C₆ alkyl)-(3- to 12-membered heterocycloalkyl), —(C₁-C₆ alkyl)-(C₆-C₁₀ aryl), or —(C₁-C₆ alkyl)-(5- to 10-membered heteroaryl) is optionally substituted with one or more R^{N2a};
each R^{N2a} independently is oxo, halogen, cyano, —OH, —NH₂, —C(═O)H, —C(═O)OH, C₁-C₆ alkyl, C₂-C₆ alkenyl, C₂-C₆ alkynyl, —O(C₁-C₆ alkyl), —NH(C₁-C₆ alkyl), —N(C₁-C₆ alkyl)₂, —C(═O)(C₁-C₆ alkyl), —C(═O)O(C₁-C₆ alkyl), —NHC(═O)O(C₁-C₆ alkyl), —S(═O)₂(C₁-C₆ alkyl), —S(═O)₂N(C₁-C₆ alkyl)₂, C₃-C₁₂ cycloalkyl, 3- to 12-membered heterocycloalkyl, C₆-C₁₀ aryl, 5- to 10-membered heteroaryl, —(C₁-C₆ alkyl)-(C₃-C₁₂ cycloalkyl), —(C₁-C₆ alkyl)-(3- to 12-membered heterocycloalkyl), —(C₁-C₆ alkyl)-(C₆-C₁₀ aryl), or —(C₁-C₆ alkyl)-(5- to 10-membered heteroaryl); wherein the C₁-C₆ alkyl, C₂-C₆ alkenyl, C₂-C₆ alkynyl, —O(C₁-C₆ alkyl), —NH(C₁-C₆ alkyl), —N(C₁-C₆ alkyl)₂, —C(═O)(C₁-C₆ alkyl), —C(═O)O(C₁-C₆ alkyl), —NHC(═O)O(C₁-C₆ alkyl), —S(═O)₂(C₁-C₆ alkyl), —S(═O)₂N(C₁-C₆ alkyl)₂, C₃-C₁₂ cycloalkyl, 3- to 12-membered heterocycloalkyl, C₆-C₁₀ aryl, 5- to 10-membered heteroaryl, —(C₁-C₆ alkyl)-(C₃—C₁₂ cycloalkyl), —(C₁-C₆ alkyl)-(3- to 12-membered heterocycloalkyl), —(C₁-C₆ alkyl)-(C₆-C₁₀ aryl), or —(C₁-C₆ alkyl)-(5- to 10-membered heteroaryl) is optionally substituted with one or more R^{N2ab}; and
each R^{N2ab} independently is oxo, halogen, cyano, —OH, —NH₂, —C(═O)H, —C(═O)OH, —O(C₁-C₆ alkyl), —NH(C₁-C₆ alkyl), —N(C₁-C₆ alkyl)₂, —C(═O)(C₁-C₆ alkyl), —C(═O)O(C₁-C₆ alkyl), —NHC(═O)O(C₁-C₆ alkyl), —S(═O)₂(C₁-C₆ alkyl), or —S(═O)₂N(C₁-C₆ alkyl)₂; or
R^{N1} and R^{N2}, together with the atom they attach to, form 3- to 12-membered heterocycloalkyl optionally substituted with one or more R^{b};
each R^{b1} independently is oxo, halogen, cyano, —OH, —NH₂, —C(═O)H, —C(═O)OH, C₁-C₆ alkyl, —O(C₁-C₆ alkyl), —NH(C₁-C₆ alkyl), —N(C₁-C₆ alkyl)₂, —C(═O)(C₁-C₆ alkyl), —C(═O)O(C₁-C₆ alkyl), —NHC(═O)O(C₁-C₆ alkyl), —S(═O)₂(C₁-C₆ alkyl), or —S(═O)₂N(C₁-C₆ alkyl)₂, wherein the C₁-C₆ alkyl, —O(C₁-C₆ alkyl), —NH(C₁-C₆ alkyl), —N(C₁-C₆ alkyl)₂, —C(═O)(C₁-C₆ alkyl), —C(═O)O(C₁-C₆ alkyl), —NHC(═O)O(C₁-C₆ alkyl), —S(═O)₂(C₁-C₆ alkyl), or —S(═O)₂N(C₁-C₆ alkyl)₂ is optionally substituted with one or more R^{b1}; and
each R^{b1} independently is oxo, halogen, cyano, —OH, or —NH₂.

In some aspects, the present disclosure relates to a compound of Formula (III), wherein:
R¹ is C₁-C₆ alkyl or C₂-C₆ alkenyl, wherein the C₁-C₆ alkyl or C₂-C₆ alkenyl is optionally substituted with one or more R^{1S};
each R^{1S} independently is halogen or C₁-C₆ alkyl;
each R² independently is H, halogen, cyano, C₁-C₆ alkyl, or C₃-C₁₂ cycloalkyl, wherein the C₁-C₆ alkyl or C₃-C₁₂ cycloalkyl is optionally substituted with one or more R^{2S}.
each R^{2S} independently is halogen, —O(C₁-C₆ alkyl), or C₃-C₁₂ cycloalkyl;
each R^{2a} independently is H, C₁-C₆ alkyl, C₁-C₆ haloalkyl, or —(CH₂)₀₋₃—C₃-C₁₂ cycloalkyl;
each R^{a} independently is H; or two R^{a}, together with the atom they attach to, form C₃-C₁₂ cycloalkyl;
R^{N2} is 3- to 12-membered heterocycloalkyl or 5- to 10-membered heteroaryl, wherein the 3- to 12-membered heterocycloalkyl or 5- to 10-membered heteroaryl is optionally substituted with one or more R^{N2a}; and
each R^{N2a} independently is halogen, C₁-C₆ alkyl, or C₃-C₁₂ cycloalkyl.

In some aspects, the present disclosure relates to a compound of Formula (III), wherein:
R¹ is C₁-C₆ alkyl, C₂-C₆ alkenyl, or C₃-C₇ cycloalkyl, wherein the C₁-C₆ alkyl is optionally substituted with one or more R^{1S};
each R^{1S} independently is halogen;
each R² independently is H, halogen, cyano, —NH₂, C₁-C₆ alkyl, —O(C₁-C₆ alkyl), —NH(C₁-C₆ alkyl), —N(C₁-C₆ alkyl)₂, C₃-C₁₂ cycloalkyl, wherein the C₁-C₆ alkyl and —NH(C₁-C₆ alkyl) is optionally substituted with one or more R^{2S};
each R^{2S} independently is halogen, —O(C₁-C₆ alkyl or —NH₂;
each R^{2a} independently is C₁-C₆ alkyl or —(CH₂)₀₋₃—C₃-C₁₂ cycloalkyl;
R^{N2} is C₃-C₁₂ cycloalkyl, 3- to 12-membered heterocycloalkyl or 5- to 10-membered heteroaryl, wherein the C₃-C₁₂ cycloalkyl, 3- to 12-membered heterocycloalkyl or 5- to 10-membered heteroaryl is optionally substituted with one or more R^{N2a}.
each R^{N2a} independently is halogen, cyano, —OH, C₁-C₆ alkyl, C₃-C₁₂ cycloalkyl, —C(═O)O(C₁-C₆ alkyl), wherein the C₁-C₆ alkyl is optionally substituted with one or more R^{N2ab}.; and
each R^{N2ab} independently is —C(═O)O(C₁-C₆ alkyl).

It is understood that, for a compound of Formula (III), A₂, A₃, A₄, R¹, R^{1S}, R², R^{2S}, R^{2a}, n, R^{a}, R^{N2}, R^{N2a}, and R^{N2ab}, can each be, where applicable, selected from the groups described herein, and any group described herein for any of A₂, A₃, A₄, R¹, R^{1S}, R², R^{2S}, R^{2a}, n, R^{a}, R^{N2}, R^{N2a}, and R^{N2ab}, can be combined, where applicable, with any group described herein for one or more of the remainder of A₂, A₃, A₄, R¹, R^{1S}, R², R^{2S}, R^{2a}, n, R^{a}, R^{N2}, R^{N2a}, and R^{N2ab}.

Variables A₂, A₃, and A₄

In some embodiments, each [symbol] independently is a single bond or double bond as valency permits.

In some embodiments, each [symbol] independently is a single bond as valency permits.

In some embodiments, each [symbol] independently is a double bond as valency permits.

In some embodiments, A₂ is CR², N, NR^{2a}, O, or S, as valency allows.

In some embodiments, A₂ is CR², N, O, or S.

In some embodiments, A₂ is CR².

In some embodiments, A₂ is CH.

In some embodiments, A₂ is NR^{2a}.

In some embodiments, A₂ is N(CH₃).

In some embodiments, A₂ is N, O, or S.

In some embodiments, A₂ is N. In some embodiments, A₂ is O. In some embodiments, A₂ is S.

In some embodiments, A₂ is CH, N(CH₃), N, O, or S, as valency allows.

In some embodiments, A₃ is CR², N, NR^{2a}, O, or S, as valency allows.

In some embodiments, A₃ is CR², N, O, or S.

In some embodiments, A₃ is CR².

In some embodiments, A₂ is CH, C(Cl), C(Br), C(CN), C(CH₃), C(CH₂CH₃), C(CH₂OCH₃), C(cyclopropyl), C(NH(CH₂CH₃)), C(NH(i-propyl)), C(NH(CH₃)), C(CH₂CF₃), C(NH(CH₂CHF₂)), C(NH(CH₂CH₂OCH₃)), C(N(CH₃)(CH₂CH₃)), C(i-propyl), C(NH₂), C(OCH₃), C(OCH₂CH₃), or C(CH₂NH₂).

In some embodiments, A₃ is NR^{2a}.

In some embodiments, A₃ is N(CH₃), N(CH₂CH₃), N(i-propyl), N(cyclobutyl), N(cyclopropyl).

In some embodiments, A₃ is N, O, or S.

In some embodiments, A₃ is N. In some embodiments, A₃ is O. In some embodiments, A₃ is S.

In some embodiments, A₂ is N(CH₃), N(CH₂CH₃), N, N(i-propyl), N(cyclopropyl), N(cyclobutyl), C(Cl), C(Br), C(CN), C(CH₃), C(CH₂CH₃), C(CH₂OCH₃), C(cyclopropyl), C(NH(CH₂CH₃)), C(NH(i-propyl)), C(NH(CH₃)), C(CH₂CF₃), C(NH(CH₂CHF₂)), C(NH(CH₂CH₂OCH₃)), C(N(CH₃)(CH₂CH₃)), C(i-propyl), C(NH₂), C(OCH₃), C(OCH₂CH₃), C(CH₂NH₂), or CH, as valency allows.

In some embodiments, A₄ is CR², N, NR^{2a}, O, or S, as valency allows.

In some embodiments, A₄ is CR², N, O, or S.

In some embodiments, A₄ is CR².

In some embodiments, A₄ is CH.

In some embodiments, A₄ is NR^{2a}.

In some embodiments, A₄ is N(CH₃).

In some embodiments, A₄ is N, O, or S.

In some embodiments, A₄ is N. In some embodiments, A₄ is O. In some embodiments, A₄ is S.

In some embodiments, A₄ is CH, N, N(CH₃), O, or S, as valency allows.

In some embodiments, at least one of A₂, A₃, or A₄ is N, NR^{2a}, O, or S.

In some embodiments, at least one of A₂, A₃, or A₄ is N, O, or S.

In some embodiments, A₂ is S, A₃ is CR², and A₄ is CR².

In some embodiments, A₂ is CR², A₃ is CR², and A₄ is S.

In some embodiments, A₂ is N, A₃ is NR^{2a}, and A₄ is CR².

In some embodiments, A₂ is O, A₃ is CR², and A₄ is CR².

In some embodiments, A₂ is NR^{2a}, A₃ is N, and A₄ is CR².

In some embodiments, A₂ is N, A₃ is CR², and A₄ is NR^{2a}.

In some embodiments, A₂ is CR², A₃ is N, and A₄ is NR^{2a}.

In some embodiments, A₂ is CR², A₃ is NR^{2a}, and A₄ is N.

In some embodiments, A₂ is CR², A₃ is CR², and A₄ is O.

In some embodiments, A₄ is CR², NR^{2a}, O, or S, as valency allows.

In some embodiments, A₄ is not N.

In some embodiments, when A₂ is S then A₄ is CR², NR^{2a}, O, or S, as valency allows.

In some embodiments, when A₂ is S then A₄ is not N.

Variables R¹ and R^{1S}

In some embodiments, R¹ is H.

In some embodiments, R¹ is —N(C₁-C₆ alkyl)₂, C₁-C₆ alkyl, C₂-C₆ alkenyl, or C₃-C₁₂ cycloalkyl, wherein the —N(C₁-C₆ alkyl)₂, C₁-C₆ alkyl, C₂-C₆ alkenyl, or C₃-C₁₂ cycloalkyl is optionally substituted with one or more R^{1S}.

In some embodiments, R¹ is —N(C₁-C₆ alkyl)₂, C₁-C₆ alkyl, C₂-C₆ alkenyl, or C₃-C₇ cycloalkyl, wherein the —N(C₁-C₆ alkyl)₂, C₁-C₆ alkyl, C₂-C₆ alkenyl, or C₃-C₇ cycloalkyl is optionally substituted with one or more R^{1S}

In some embodiments, R¹ is —N(C₁-C₆ alkyl)₂ optionally substituted with one or more R^{1S}.

In some embodiments, R¹ is —N(C₁-C₆ alkyl)₂.

In some embodiments, R¹ is —N(CH₃)₂.

In some embodiments, R¹ is C₁-C₆ alkyl optionally substituted with one or more R^{1S}.

In some embodiments, R¹ is C₁-C₆ alkyl substituted with one or more R^{1S}.

In some embodiments, R¹ is propyl (e.g., isopropyl).

In some embodiments, R¹ is C₁-C₆ alkyl substituted with one or more R^{1S}.

In some embodiments, R¹ is propyl (e.g., isopropyl) substituted with one or more R^{1S}.

In some embodiments, R¹ is C₂-C₆ alkenyl optionally substituted with one or more R^{1S}.

In some embodiments, R¹ is C₂-C₆ alkenyl substituted with one or more R^{1S}.

In some embodiments, R¹ is propenyl (e.g., isopropenyl).

In some embodiments, R¹ is C₂-C₆ alkenyl substituted with one or more R^{1S}.

In some embodiments, R¹ is propenyl (e.g., isopropenyl) substituted with one or more R^{1S}.

In some embodiments, R¹ is C₃-C₁₂ cycloalkyl optionally substituted with one or more R^{1S}.

In some embodiments, R¹ is C₃-C₁₂ cycloalkyl substituted with one or more R^{1S}.

In some embodiments, R¹ is C₃-C₁₂ cycloalkyl.

In some embodiments, R¹ is C₃-C₇ cycloalkyl optionally substituted with one or more R^{1S}.

In some embodiments, R¹ is C₃-C₇ cycloalkyl substituted with one or more R^{1S}.

In some embodiments, R¹ is C₃-C₇ cycloalkyl.

In some embodiments, R¹ is cyclopropyl.

In some embodiments, R¹ is cyclopropyl substituted with one or more R^{1S}.

In some embodiments, at least one R^{1S} is halogen.

In some embodiments, at least one R^{1S} is F, Cl, or Br.

In some embodiments, at least one R^{1S} is F.

In some embodiments, at least one R^{1S} is Cl.

In some embodiments, at least one R^{1S} is Br.

In some embodiments, at least one R^{1S} is cyano.

In some embodiments, at least one R^{1S} is —OH.

In some embodiments, at least one R^{1S} is C₁-C₆ alkyl.

In some embodiments, R¹ is C₁-C₆ alkyl, C₂-C₆ alkenyl or C₃-C₇ cycloalkyl, wherein C₁-C₆ alkyl is optionally substituted with one or more R^{1S}.

In some embodiments, R¹ is C₁-C₆ alkyl (e.g., methyl, ethyl or isopropyl), C₂-C₆ alkenyl (e.g., isopropenyl), C₃-C₇ cycloalkyl (e.g., cyclopropyl) or C₆ alkyl is optionally substituted with one or more R^{1S}(e.g., fluoromethyl).

In some embodiments, R¹ is methyl, ethyl, isopropyl, isopropenyl, cyclopropyl or fluoromethyl.

Variables R², R^{2S}, and R^{2a}

In some embodiments, each R² independently is H.

In some embodiments, each R² independently is halogen.

In some embodiments, each R² independently is cyano.

In some embodiments, each R² independently is —OH or —NH₂.

In some embodiments, each R² independently is —NO₂.

In some embodiments, each R² independently is —C(═O)NH₂.

In some embodiments, each R² independently is C₁-C₆ alkyl, C₂-C₆ alkenyl, or C₂-C₆ alkynyl, wherein the C₁-C₆ alkyl, C₂-C₆ alkenyl, or C₂-C₆ alkynyl is optionally substituted with one or more R^{2S}.

In some embodiments, each R² independently is C₁-C₆ alkyl optionally substituted with one or more R^{2S}.

In some embodiments, each R² independently is C₁-C₆ alkyl.

In some embodiments, each R² independently is C₁-C₆ alkyl substituted with one or more R^{2S}.

In some embodiments, each R² independently is C₂-C₆ alkenyl optionally substituted with one or more R^{2S}.

In some embodiments, each R² independently is C₂-C₆ alkenyl.

In some embodiments, each R² independently is C₂-C₆ alkenyl substituted with one or more R^{2S}.

In some embodiments, each R² independently is C₂-C₆ alkynyl optionally substituted with one or more R^{2S}.

In some embodiments, each R² independently is C₂-C₆ alkynyl.

In some embodiments, each R² independently is C₂-C₆ alkynyl substituted with one or more R^{2S}.

In some embodiments, each R² independently is —O(C₁-C₆ alkyl), —NH(C₁-C₆ alkyl), or —N(C₁-C₆ alkyl)₂, wherein the —O(C₁-C₆ alkyl), —NH(C₁-C₆ alkyl), or —N(C₁-C₆ alkyl)₂ is optionally substituted with one or more R^{2S}.

In some embodiments, each R² independently is —O(C₁-C₆ alkyl) optionally substituted with one or more R^{2S}.

In some embodiments, each R² independently is —O(C₁-C₆ alkyl).

In some embodiments, each R² independently is —NH(C₁-C₆ alkyl) or —N(C₁-C₆ alkyl)₂ optionally substituted with one or more R^{2S}.

In some embodiments, each R² independently is —NH(C₁-C₆ alkyl) or —N(C₁-C₆ alkyl)₂.

In some embodiments, each R² independently is C₃-C₁₂ cycloalkyl, 3- to 12-membered heterocycloalkyl, C₆-C₁₀ aryl, or 5- to 10-membered heteroaryl, wherein the C₃-C₁₂ cycloalkyl, 3- to 12-membered heterocycloalkyl, C₆-C₁₀ aryl, or 5- to 10-membered heteroaryl is optionally substituted with one or more R^{2S}.

In some embodiments, each R² independently is C₃-C₇ cycloalkyl, 3- to 12-membered heterocycloalkyl, C₆-C₁₀ aryl, or 5- to 10-membered heteroaryl, wherein the C₃-C₇ cycloalkyl, 3- to 12-membered heterocycloalkyl, C₆-C₁₀ aryl, or 5- to 10-membered heteroaryl is optionally substituted with one or more R^{2S}.

In some embodiments, each R² independently is C₃-C₁₂ cycloalkyl optionally substituted with one or more R^{2S}.

In some embodiments, each R² independently is C₃-C₁₂ cycloalkyl.

In some embodiments, each R² independently is C₃-C₁₂ cycloalkyl substituted with one or more R^{2S}.

In some embodiments, each R² independently is C₃-C₇ cycloalkyl optionally substituted with one or more R^{2S}.

In some embodiments, each R² independently is C₃-C₇ cycloalkyl.

In some embodiments, each R² independently is C₃-C₇ cycloalkyl substituted with one or more R^{2S}.

In some embodiments, each R² independently is 3- to 12-membered heterocycloalkyl optionally substituted with one or more R^{2S}.

In some embodiments, each R² independently is 3- to 12-membered heterocycloalkyl.

In some embodiments, each R² independently is 3- to 12-membered heterocycloalkyl substituted with one or more R^{2S}.

In some embodiments, each R² independently is C₆-C₁₀ aryl optionally substituted with one or more R^{2S}.

In some embodiments, each R² independently is C₆-C₁₀ aryl.

In some embodiments, each R² independently is C₆-C₁₀ aryl substituted with one or more R^{2S}.

In some embodiments, each R² independently is 5- to 10-membered heteroaryl optionally substituted with one or more R^{2S}.

In some embodiments, each R² independently is 5- to 10-membered heteroaryl.

In some embodiments, each R² independently is 5- to 10-membered heteroaryl substituted with one or more R^{2S}.

In some embodiments, two R² together with the atoms to which they are attached form a C₃-C₁₂ cycloalkyl or 3- to 12-membered heterocycloalkyl, wherein the C₃-C₁₂ cycloalkyl or 3- to 12-membered heterocycloalkyl is optionally substituted with one or more R^{2S}.

In some embodiments, two R² together with the atoms to which they are attached form a C₃-C₇ cycloalkyl or 3- to 12-membered heterocycloalkyl, wherein the C₃-C₇ cycloalkyl or 3- to 12-membered heterocycloalkyl is optionally substituted with one or more R^{2S}.

In some embodiments, two R² together with the atoms to which they are attached form a C₃-C₁₂ cycloalkyl or 3- to 12-membered heterocycloalkyl.

In some embodiments, two R² together with the atoms to which they are attached form a C₃-C₁₂ cycloalkyl optionally substituted with one or more R^{2S}.

In some embodiments, two R² together with the atoms to which they are attached form a C₃-C₁₂ cycloalkyl.

In some embodiments, two R² together with the atoms to which they are attached form a C₃-C₇ cycloalkyl or 3- to 12-membered heterocycloalkyl.

In some embodiments, two R² together with the atoms to which they are attached form a C₃-C₇ cycloalkyl optionally substituted with one or more R^{2S}.

In some embodiments, two R² together with the atoms to which they are attached form a C₃-C₇ cycloalkyl.

In some embodiments, two R² together with the atoms to which they are attached form a 3- to 12-membered heterocycloalkyl optionally substituted with one or more R^{2S}.

In some embodiments, two R² together with the atoms to which they are attached form a 3- to 12-membered heterocycloalkyl.

In some embodiments, R² independently is H, halogen, cyano, —NH₂, C₁-C₆ alkyl —O(C₁-C₆ alkyl), —NH(C₁-C₆ alkyl), —N(C₁-C₆ alkyl)₂, C₃-C₁₂ cycloalkyl, wherein the C₁-C₆ alkyl and —NH(C₁-C₆ alkyl) is optionally substituted with one or more R^{2S}.

In some embodiments, R² independently is H, halogen (e.g., chlorine or bromine), cyano, —NH₂, C₁-C₆ alkyl (e.g., methyl, ethyl, or propyl), —O(C₁-C₆ alkyl) (e.g., —O-methyl or —O-ethyl), —NH(C₁-C₆ alkyl) (e.g., —NH-methyl, —NH-CD₃, —NH-ethyl, or —NH-isopropyl), —N(C₁-C₆ alkyl)₂ (e.g., (—N(Me)(Et)) or C₃-C₁₂ cycloalkyl (e.g., cyclopropyl), wherein the C₁-C₆ alkyl and —NH(C₁-C₆ alkyl) is optionally substituted with one or more R^{2S} (e.g., —CH₂—CF₃, —NHCH₂CHF₂, —CH₂—O-methyl, —NHCH₂CH₂OMe, or —CH₂—NH₂).

In some embodiments, R² independently is H, chlorine, bromine, cyano, —NH₂, methyl, ethyl, propyl, —O-methyl, —O-ethyl, —NH-methyl, —NH-CD₃, —NH-ethyl, —NH— isopropyl, —N(Me)(Et), cyclopropyl, —CH₂—CF₃, —NHCH₂CHF₂, —CH₂—O-methyl, —NHCH₂CH₂OMe, or —CH₂—NH₂.

In some embodiments, at least one R^{2S} is halogen.

In some embodiments, at least one R^{2S} is F, Cl, or Br.

In some embodiments, at least one R^{2S} is F.

In some embodiments, at least one R^{2S} is Cl.

In some embodiments, at least one R^{2S} is Br.

In some embodiments, at least one R^{2S} is —OH.

In some embodiments, at least one R^{2S} is —O(C₁-C₆ alkyl).

In some embodiments, at least one R^{2S} is —NH₂.

In some embodiments, at least one R^{2S} is —NH(C₁-C₆ alkyl) or —N(C₁-C₆ alkyl)₂.

In some embodiments, at least one R^{2S} is C₃-C₁₂ cycloalkyl.

In some embodiments, at least one R^{2S} is C₃-C₇ cycloalkyl.

In some embodiments, at least one R^{2S} is C₃ cycloalkyl. In some embodiments, at least one R^{2S} is C₄ cycloalkyl. In some embodiments, at least one R^{2S} is C₅ cycloalkyl. In some embodiments, at least one R^{2S} is C₆ cycloalkyl. In some embodiments, at least one R^{2S} is C₇ cycloalkyl. In some embodiments, at least one R^{2S} is C₅ cycloalkyl. In some embodiments, at least one R^{2S} is C₉ cycloalkyl. In some embodiments, at least one R^{2S} is C₁₀ cycloalkyl. In some embodiments, at least one R^{2S} is C₁₁ cycloalkyl. In some embodiments, at least one R^{2S} is C₁₂ cycloalkyl.

In some embodiments, each R^{2a} independently is H.

In some embodiments, each R^{2a} independently is H, C₁-C₆ alkyl, C₂-C₆ alkenyl, C₂-C₆ alkynyl, C₁-C₆ haloalkyl, —(CH₂)₀₋₃—(C₃-C₁₂ cycloalkyl), or —(CH₂)₀₋₃-(3- to 12-membered heterocycloalkyl).

In some embodiments, each R^{2a} independently is C₁-C₆ alkyl.

In some embodiments, each R^{2a} independently is methyl, ethyl, or isopropyl.

In some embodiments, each R^{2a} independently is C₂-C₆ alkenyl.

In some embodiments, each R^{2a} independently is C₂-C₆ alkynyl.

In some embodiments, each R^{2a} independently is C₁-C₆ haloalkyl.

In some embodiments, each R^{2a} independently is —CF₃.

In some embodiments, each R^{2a} independently is —(CH₂)₀₋₃—(C₃-C₁₂ cycloalkyl) or —(CH₂)₀₋₃-(3- to 12-membered heterocycloalkyl).

In some embodiments, each R^{2a} independently is —(CH₂)₀₋₃—(C₃-C₁₂ cycloalkyl).

In some embodiments, each R^{2a} independently is C₃-C₁₂ cycloalkyl.

In some embodiments, each R^{2a} independently is C₃-C₇ cycloalkyl.

In some embodiments, each R^{2a} independently is —(CH₂)₀₋₃-(3- to 12-membered heterocycloalkyl).

In some embodiments, each R^{2a} independently is −3- to 12-membered heterocycloalkyl.

In some embodiments, each R^{2a} independently is C₁-C₆ alkyl or —(CH₂)₀₋₃—C₃-C₁₂ cycloalkyl.

In some embodiments, each R^{2a} independently is C₁-C₆ alkyl (e.g., methyl, ethyl, or isopropyl) or —(CH₂)₀₋₃—C₃-C₁₂ cycloalkyl (e.g., cyclopropyl or cyclobutyl).

In some embodiments, each R^{2a} independently is methyl, ethyl, isopropyl, cyclopropyl, or cyclobutyl.

### Variables R^{a}

In some embodiments, at least one R^{a} is H.

In some embodiments, both R^{a} are H.

In some embodiments, at least one R^{a} is C₁-C₆ alkyl (e.g., methyl, ethyl, or propyl).

In some embodiments, at least one R^{a} is C₁-C₄ alkyl (e.g., methyl, ethyl, or propyl).

In some embodiments, one R^{a} is H, and the other R^{a} is C₁-C₆ alkyl (e.g., methyl, ethyl, or propyl).

In some embodiments, one R^{a} is H, and the other R^{a} is C₁-C₄ alkyl (e.g., methyl, ethyl, or propyl).

In some embodiments, two R^{a}, together with the atom they attach to, form C₃-C₁₂ cycloalkyl.

In some embodiments, two R^{a}, together with the atom they attach to, form C₃-C₇ cycloalkyl.

In some embodiments, two R^{a}, together with the atom they attach to, form C₃-C₆ cycloalkyl.

In some embodiments, two R^{a}, together with the atom they attach to, form cyclopropyl, cyclobutyl, or cyclopentyl, or cyclohexyl.

In some embodiments, two R^{a}, together with the atom they attach to, form cyclopropyl.

In some embodiments, both R^{a} are H or two R^{a}, together with the atom they attach to, form C₃-C₁₂ cycloalkyl (e.g., C₃-C₇ cycloalkyl or C₃-C₆ cycloalkyl).

In some embodiments, both R^{a} are H or two R^{a}, together with the atom they attach to, form cyclopropyl.

Variables R^{N2}, R^{Na}, and R^{N2ab}.

In some embodiments, R^{N2} is C₁-C₆ alkyl, C₂-C₆ alkenyl, C₂-C₆ alkynyl, —O—(C₁-C₆ alkyl), —O—(C₂-C₆ alkenyl), —O—(C₂-C₆ alkynyl), —NH—(C₁-C₆ alkyl), —NH—(C₂-C₆ alkenyl), or —NH—(C₂-C₆ alkynyl), wherein the C₁-C₆ alkyl, C₂-C₆ alkenyl, C₂-C₆ alkynyl, —O—(C₁-C₆ alkyl), —O—(C₂-C₆ alkenyl), —O—(C₂-C₆ alkynyl)-NH—(C₁-C₆ alkyl), —NH—(C₂-C₆ alkenyl), or —NH—(C₂-C₆ alkynyl) is optionally substituted with one or more R^{N2a}.

In some embodiments, R^{N2} is C₁-C₆ alkyl optionally substituted with one or more R^{N2a}.

In some embodiments, R^{N2} is C₁-C₆ alkyl.

In some embodiments, R^{N2} is C₁-C₆ alkyl substituted with one or more R^{N2a}.

In some embodiments, R^{N2} is C₂-C₆ alkenyl optionally substituted with one or more R^{N2a}.

In some embodiments, R^{N2} is C₂-C₆ alkenyl.

In some embodiments, R^{N2} is C₂-C₆ alkenyl substituted with one or more R^{N2a}.

In some embodiments, R^{N2} is C₂-C₆ alkynyl optionally substituted with one or more R^{N2a}.

In some embodiments, R^{N2} is C₂-C₆ alkynyl.

In some embodiments, R^{N2} is C₂-C₆ alkynyl substituted with one or more R^{N2a}.

In some embodiments, R^{N2} is —O—(C₁-C₆ alkyl) optionally substituted with one or more R^{N2a}.

In some embodiments, R^{N2} is —O—(C₁-C₆ alkyl).

In some embodiments, R^{N2} is —O—(C₁-C₆ alkyl) substituted with one or more R^{N2a}.

In some embodiments, R^{N2} is —O—(C₂-C₆ alkenyl) optionally substituted with one or more R^{N2a}.

In some embodiments, R^{N2} is —O—(C₂-C₆ alkenyl).

In some embodiments, R^{N2} is —O—(C₂-C₆ alkenyl) substituted with one or more R^{N2a}.

In some embodiments, R^{N2} is —O—(C₂-C₆ alkynyl) optionally substituted with one or more R^{N2a}.

In some embodiments, R^{N2} is —O—(C₂-C₆ alkynyl).

In some embodiments, R^{N2} is —O—(C₂-C₆ alkynyl) substituted with one or more R^{N2a}.

In some embodiments, R^{N2} is —NH—(C₁-C₆ alkyl) optionally substituted with one or more R^{N2a}.

In some embodiments, R^{N2} is —NH—(C₁-C₆ alkyl).

In some embodiments, R^{N2} is —NH—(C₁-C₆ alkyl) substituted with one or more R^{N2a}.

In some embodiments, R^{N2} is —NH—(C₂-C₆ alkenyl) optionally substituted with one or more R^{N2a}.

In some embodiments, R^{N2} is —NH—(C₂-C₆ alkenyl).

In some embodiments, R^{N2} is —NH—(C₂-C₆ alkenyl) substituted with one or more R^{N2a}.

In some embodiments, R^{N2} is —NH—(C₂-C₆ alkynyl) optionally substituted with one or more R^{N2a}.

In some embodiments, R^{N2} is —NH—(C₂-C₆ alkynyl).

In some embodiments, R^{N2} is —NH—(C₂-C₆ alkynyl) substituted with one or more R^{N2a}.

In some embodiments, R^{N2} is C₃-C₁₂ cycloalkyl, 3- to 12-membered heterocycloalkyl, C₆-C₁₀ aryl, or 5- to 10-membered heteroaryl, wherein the C₃-C₁₂ cycloalkyl, 3- to 12-membered heterocycloalkyl, C₆-C₁₀ aryl, or 5- to 10-membered heteroaryl is optionally substituted with one or more R^{N2a}.

In some embodiments, R^{N2} is C₃-C₇ cycloalkyl, 3- to 12-membered heterocycloalkyl, C₆-C₁₀ aryl, or 5- to 10-membered heteroaryl, wherein the C₃-C₇ cycloalkyl, 3- to 12-membered heterocycloalkyl, C₆-C₁₀ aryl, or 5- to 10-membered heteroaryl is optionally substituted with one or more R^{N2a}.

In some embodiments, R^{N2} is C₃-C₁₂ cycloalkyl optionally substituted with one or more R^{N2a}.

In some embodiments, R^{N2} is C₃-C₁₂ cycloalkyl.

In some embodiments, R^{N2} is C₃-C₁₂ cycloalkyl substituted with one or more R^{N2a}.

In some embodiments, R^{N2} is C₃-C₇ cycloalkyl optionally substituted with one or more R^{N2a}.

In some embodiments, R^{N2} is C₃-C₇ cycloalkyl.

In some embodiments, R^{N2} is C₃-C₇ cycloalkyl substituted with one or more R^{N2a}.

In some embodiments, R^{N2} is 3- to 12-membered heterocycloalkyl optionally substituted with one or more R^{N2a}.

In some embodiments, R^{N2} is 3- to 12-membered heterocycloalkyl.

In some embodiments, R^{N2} is 3- to 12-membered heterocycloalkyl substituted with one or more R^{N2a}.

In some embodiments, R^{N2} is C₆-C₁₀ aryl optionally substituted with one or more R^{N2a}.

In some embodiments, R^{N2} is C₆-C₁₀ aryl.

In some embodiments, R^{N2} is C₆-C₁₀ aryl substituted with one or more R^{N2a}.

In some embodiments, R^{N2} is 5- to 10-membered heteroaryl optionally substituted with one or more R^{N2a}.

In some embodiments, R^{N2} is 5- to 10-membered heteroaryl.

In some embodiments, R^{N2} is 5- to 10-membered heteroaryl substituted with one or more R^{N2a}.

In some embodiments, R^{N2} is —(C₁-C₆ alkyl)-(C₃-C₁₂ cycloalkyl), —(C₁-C₆ alkyl)-(3- to 12-membered heterocycloalkyl), —(C₁-C₆ alkyl)-(C₆-C₁₀ aryl), or —(C₁-C₆ alkyl)-(5- to 10-membered heteroaryl); wherein the —(C₁-C₆ alkyl)-(C₃-C₁₂ cycloalkyl), —(C₁-C₆ alkyl)-(3- to 12-membered heterocycloalkyl), —(C₁-C₆ alkyl)-(C₆-C₁₀ aryl), or —(C₁-C₆ alkyl)-(5- to 10-membered heteroaryl) is optionally substituted with one or more R^{N2a}.

In some embodiments, R^{N2} is —(C₁-C₆ alkyl)-(C₃-C₁₂ cycloalkyl) optionally substituted with one or more R^{N2a}.

In some embodiments, R^{N2} is —(C₁-C₆ alkyl)-(C₃-C₁₂ cycloalkyl).

In some embodiments, R^{N2} is —(C₁-C₆ alkyl)-(C₃-C₁₂ cycloalkyl) substituted with one or more R^{N2a}.

In some embodiments, R^{N2} is —(C₁-C₆ alkyl)-(3- to 12-membered heterocycloalkyl) optionally substituted with one or more R^{N2a}.

In some embodiments, R^{N2} is —(C₁-C₆ alkyl)-(3- to 12-membered heterocycloalkyl).

In some embodiments, R^{N2} is —(C₁-C₆ alkyl)-(3- to 12-membered heterocycloalkyl) substituted with one or more R^{N2a}.

In some embodiments, R^{N2} is —(C₁-C₆ alkyl)-(C₆-C₁₀ aryl) optionally substituted with one or more R^{N2a}.

In some embodiments, R^{N2} is —(C₁-C₆ alkyl)-(C₆-C₁₀ aryl).

In some embodiments, R^{N2} is —(C₁-C₆ alkyl)-(C₆-C₁₀ aryl) substituted with one or more R^{N2a}.

In some embodiments, R^{N2} is —(C₁-C₆ alkyl)-(5- to 10-membered heteroaryl) optionally substituted with one or more R^{N2a}.

In some embodiments, R^{N2} is —(C₁-C₆ alkyl)-(5- to 10-membered heteroaryl).

In some embodiments, R^{N2} is —(C₁-C₆ alkyl)-(5- to 10-membered heteroaryl) substituted with one or more R^{N2a}.

In some embodiments, R^{N2} is C₃-C₁₂ cycloalkyl (e.g., cyclobutyl), 3- to 12-membered heterocycloalkyl (e.g., piperidinyl, octahydroindolizin-8-yl, or oxaspiro[3.3]heptan-6-yl) or 5- to 10-membered heteroaryl (e.g., oxazolyl, pyrimidinyl, or triazolylpyridinyl), wherein the C₃-C₁₂ cycloalkyl, 3- to 12-membered heterocycloalkyl, or 5- to 10-membered heteroaryl is optionally substituted with one or more R^{N2a}.

In some embodiments, R^{N2} is cyclobutyl, piperidinyl, octahydroindolizin-8-yl, oxaspiro[3.3]heptan-6-yl), oxazolyl, pyrimidinyl, or triazolylpyridinyl, wherein the cyclobutyl, piperidinyl, octahydroindolizin-8-yl, oxaspiro[3.3]heptan-6-yl), oxazolyl, pyrimidinyl, or triazolylpyridinyl is optionally substituted with one or more R^{N2a}.

In some embodiments, at least one R^{N2a} is oxo.

In some embodiments, at least two R^{N2a} are oxo.

In some embodiments, at least one R^{N2a} is halogen.

In some embodiments, at least one R^{N2a} is F, Cl, or Br.

In some embodiments, at least one R^{N2a} is F.

In some embodiments, at least one R^{N2a} is Cl.

In some embodiments, at least one R^{N2a} is Br.

In some embodiments, at least one R^{N2a} is cyano.

In some embodiments, at least one R^{N2a} is —OH, —NH₂, —C(═O)H, or —C(═O)OH.

In some embodiments, at least one R^{N2a} is —OH.

In some embodiments, at least one R^{N2a} is —NH₂.

In some embodiments, at least one R^{N2a} is —C(═O)H.

In some embodiments, at least one R^{N2a} is —C(═O)OH.

In some embodiments, at least one R^{N2a} is C₁-C₆ alkyl, C₂-C₆ alkenyl, or C₂-C₆ alkynyl, wherein the C₁-C₆ alkyl, C₂-C₆ alkenyl, or C₂-C₆ alkynyl is optionally substituted with one or more R^{N2ab}.

In some embodiments, at least one R^{N2a} is C₁-C₆ alkyl optionally substituted with one or more R^{N2ab}.

In some embodiments, at least one R^{N2a} is C₁-C₆ alkyl.

In some embodiments, at least one R^{N2a} is C₁-C₆ alkyl substituted with one or more R^{N2ab}.

In some embodiments, at least one R^{N2a} is C₂-C₆ alkenyl optionally substituted with one or more R^{N2ab}.

In some embodiments, at least one R^{N2a} is C₂-C₆ alkenyl.

In some embodiments, at least one R^{N2a} is C₂-C₆ alkenyl substituted with one or more R^{N2ab}.

In some embodiments, at least one R^{N2a} is C₂-C₆ alkynyl optionally substituted with one or more R^{N2ab}.

In some embodiments, at least one R^{N2a} is C₂-C₆ alkynyl.

In some embodiments, at least one R^{N2a} is C₂-C₆ alkynyl substituted with one or more R^{N2ab}.

In some embodiments, at least one R^{N2a} is —O(C₁-C₆ alkyl), —NH(C₁-C₆ alkyl), or —N(C₁-C₆ alkyl)₂, wherein the —O(C₁-C₆ alkyl), —NH(C₁-C₆ alkyl), or —N(C₁-C₆ alkyl)₂ is optionally substituted with one or more R^{N2ab}.

In some embodiments, at least one R^{N2a} is —O(C₁-C₆ alkyl) optionally substituted with one or more R^{N2ab}.

In some embodiments, at least one R^{N2a} is —O(C₁-C₆ alkyl).

In some embodiments, at least one R^{N2a} is —NH(C₁-C₆ alkyl) or —N(C₁-C₆ alkyl)₂ optionally substituted with one or more R^{N2ab}.

In some embodiments, at least one R^{N2a} is —NH(C₁-C₆ alkyl) or —N(C₁-C₆ alkyl)₂.

In some embodiments, at least one R^{N2a} is —C(═O)(C₁-C₆ alkyl), —C(═O)O(C₁-C₆ alkyl), or —NHC(═O)O(C₁-C₆ alkyl), wherein the —C(═O)(C₁-C₆ alkyl), —C(═O)O(C₁-C₆ alkyl), or —NHC(═O)O(C₁-C₆ alkyl) is optionally substituted with one or more R^{N2ab}.

In some embodiments, at least one R^{N2a} is —C(═O)(C₁-C₆ alkyl) optionally substituted with one or more R^{N2ab}.

In some embodiments, at least one R^{N2a} is —C(═O)(C₁-C₆ alkyl).

In some embodiments, at least one R^{N2a} is —C(═O)(C₁-C₆ alkyl) substituted with one or more R^{N2ab}.

In some embodiments, at least one R^{N2a} is —C(═O)O(C₁-C₆ alkyl) optionally substituted with one or more R^{N2ab}.

In some embodiments, at least one R^{N2a} is —C(═O)O(C₁-C₆ alkyl).

In some embodiments, at least one R^{N2a} is —C(═O)O(C₁-C₆ alkyl) substituted with one or more R^{N2ab}.

In some embodiments, at least one R^{N2a} is —NHC(═O)O(C₁-C₆ alkyl) optionally substituted with one or more R^{N2ab}.

In some embodiments, at least one R^{N2a} is —NHC(═O)O(C₁-C₆ alkyl).

In some embodiments, at least one R^{N2a} is —NHC(═O)O(C₁-C₆ alkyl) substituted with one or more R^{N2ab}.

In some embodiments, at least one R^{N2a} is —S(═O)₂(C₁-C₆ alkyl) or —S(═O)₂N(C₁-C₆ alkyl)₂, wherein the —S(═O)₂(C₁-C₆ alkyl) or —S(═O)₂N(C₁-C₆ alkyl)₂ is optionally substituted with one or more R^{N2ab}.

In some embodiments, at least one R^{N2a} is —S(═O)₂(C₁-C₆ alkyl) optionally substituted with one or more R^{N2ab}.

In some embodiments, at least one R^{N2a} is —S(═O)₂(C₁-C₆ alkyl).

In some embodiments, at least one R^{N2a} is —S(═O)₂(C₁-C₆ alkyl) substituted with one or more R^{N2ab}.

In some embodiments, at least one R^{N2a} is —S(═O)₂N(C₁-C₆ alkyl)₂ optionally substituted with one or more R^{N2ab}.

In some embodiments, at least one R^{N2a} is —S(═O)₂N(C₁-C₆ alkyl)₂.

In some embodiments, at least one R^{N2a} is —S(═O)₂N(C₁-C₆ alkyl)₂ substituted with one or more R^{N2ab}.

In some embodiments, at least one R^{N2a} is C₃-C₁₂ cycloalkyl, 3- to 12-membered heterocycloalkyl, C₆-C₁₀ aryl, or 5- to 10-membered heteroaryl, wherein the C₃-C₁₂ cycloalkyl, 3- to 12-membered heterocycloalkyl, C₆-C₁₀ aryl, or 5- to 10-membered heteroaryl is optionally substituted with one or more R^{N2ab}.

In some embodiments, at least one R^{N2a} is C₃-C₇ cycloalkyl, 3- to 12-membered heterocycloalkyl, C₆-C₁₀ aryl, or 5- to 10-membered heteroaryl, wherein the C₃-C₇ cycloalkyl, 3- to 12-membered heterocycloalkyl, C₆-C₁₀ aryl, or 5- to 10-membered heteroaryl is optionally substituted with one or more R^{N2ab}.

In some embodiments, at least one R^{N2a} is C₃-C₁₂ cycloalkyl optionally substituted with one or more R^{N2ab}.

In some embodiments, at least one R^{N2a} is C₃-C₁₂ cycloalkyl.

In some embodiments, at least one R^{N2a} is C₃-C₁₂ cycloalkyl substituted with one or more R^{N2ab}.

In some embodiments, at least one R^{N2a} is C₃-C₇ cycloalkyl optionally substituted with one or more R^{N2ab}.

In some embodiments, at least one R^{N2a} is C₃-C₇ cycloalkyl.

In some embodiments, at least one R^{N2a} is C₃-C₇ cycloalkyl substituted with one or more R^{N2ab}.

In some embodiments, at least one R^{N2a} is 3- to 12-membered heterocycloalkyl optionally substituted with one or more R^{N2ab}.

In some embodiments, at least one R^{N2a} is 3- to 12-membered heterocycloalkyl.

In some embodiments, at least one R^{N2a} is 3- to 12-membered heterocycloalkyl substituted with one or more R^{N2ab}.

In some embodiments, at least one R^{N2a} is C₆-C₁₀ aryl optionally substituted with one or more R^{N2ab}.

In some embodiments, at least one R^{N2a} is C₆-C₁₀ aryl.

In some embodiments, at least one R^{N2a} is C₆-C₁₀ aryl substituted with one or more R^{N2ab}.

In some embodiments, at least one R^{N2a} is 5- to 10-membered heteroaryl optionally substituted with one or more R^{N2ab}.

In some embodiments, at least one R^{N2a} is 5- to 10-membered heteroaryl.

In some embodiments, at least one R^{N2a} is 5- to 10-membered heteroaryl substituted with one or more R^{N2ab}.

In some embodiments, at least one R^{N2a} is —(C₁-C₆ alkyl)-(C₃-C₁₂ cycloalkyl), —(C₁-C₆ alkyl)-(3- to 12-membered heterocycloalkyl), —(C₁-C₆ alkyl)-(C₆-C₁₀ aryl), or —(C₁-C₆ alkyl)-(5- to 10-membered heteroaryl), wherein the —(C₁-C₆ alkyl)-(C₃-C₁₂ cycloalkyl), —(C₁-C₆ alkyl)-(3- to 12-membered heterocycloalkyl), —(C₁-C₆ alkyl)-(C₆-C₁₀ aryl), or —(C₁-C₆ alkyl)-(5- to 10-membered heteroaryl) is optionally substituted with one or more R^{N2ab}.

In some embodiments, at least one R^{N2a} is —(C₁-C₆ alkyl)-(C₃-C₁₂ cycloalkyl) optionally substituted with one or more R^{N2ab}.

In some embodiments, at least one R^{N2a} is —(C₁-C₆ alkyl)-(C₃-C₁₂ cycloalkyl).

In some embodiments, at least one R^{N2a} is —(C₁-C₆ alkyl)-(C₃-C₁₂ cycloalkyl) substituted with one or more R^{N2ab}.

In some embodiments, at least one R^{N2a} is —(C₁-C₆ alkyl)-(3- to 12-membered heterocycloalkyl) optionally substituted with one or more R^{N2ab}.

In some embodiments, at least one R^{N2a} is —(C₁-C₆ alkyl)-(3- to 12-membered heterocycloalkyl).

In some embodiments, at least one R^{N2a} is —(C₁-C₆ alkyl)-(3- to 12-membered heterocycloalkyl) substituted with one or more R^{N2ab}.

In some embodiments, at least one R^{N2a} is —(C₁-C₆ alkyl)-(C₆-C₁₀ aryl) optionally substituted with one or more R^{N2ab}.

In some embodiments, at least one R^{N2a} is —(C₁-C₆ alkyl)-(C₆-C₁₀ aryl).

In some embodiments, at least one R^{N2a} is —(C₁-C₆ alkyl)-(C₆-C₁₀ aryl) substituted with one or more R^{N2ab}.

In some embodiments, at least one R^{N2a} is —(C₁-C₆ alkyl)-(5- to 10-membered heteroaryl) optionally substituted with one or more R^{N2ab}.

In some embodiments, at least one R^{N2a} is —(C₁-C₆ alkyl)-(5- to 10-membered heteroaryl).

In some embodiments, at least one R^{N2a} is —(C₁-C₆ alkyl)-(5- to 10-membered heteroaryl) substituted with one or more R^{N2ab}.

In some embodiments, R^{N2a} independently is halogen (e.g., F or Cl), cyano, —OH, C₁-C₆ alkyl (e.g., methyl), C₃-C₁₂ cycloalkyl (e.g., cyclopropyl or cyclobutyl), —C(═O)O(C₁-C₆ alkyl) (e.g., —COO-ethyl), wherein the C₁-C₆ alkyl (e.g., methyl) is optionally substituted with one or more R^{N2ab} (e.g., —C(═O)O(C₁-C₆ alkyl)).

In some embodiments, R^{N2a} independently is halogen (e.g., F or Cl), cyano, —OH, C₁-C₆ alkyl (e.g., methyl), C₃-C₁₂ cycloalkyl (e.g., cyclopropyl or cyclobutyl), —C(═O)O(C₁-C₆ alkyl) (e.g., —COO-ethyl), wherein the C₁-C₆ alkyl (e.g., methyl) is optionally substituted with one or more R^{N2ab} (e.g., —C(═O)O(ethyl)).

In some embodiments, R^{N2a} independently is F, C₁, cyano, —OH, methyl, cyclopropyl, cyclobutyl, or —COO-ethyl, wherein the methyl is optionally substituted with one or more —C(═O)O(ethyl).

In some embodiments, R^{N2a} independently is F, Cl, cyano, —OH, methyl, cyclopropyl, cyclobutyl, or —COO-ethyl.

In some embodiments, at least one R^{N2ab} is oxo.

In some embodiments, at least two R^{N2ab} are oxo.

In some embodiments, at least one R^{N2ab} is halogen.

In some embodiments, at least one R^{N2ab} is F, Cl, or Br.

In some embodiments, at least one R^{N2ab} is F.

In some embodiments, at least one R^{N2ab} is Cl.

In some embodiments, at least one R^{N2ab} is Br.

In some embodiments, at least one R^{N2ab} is cyano.

In some embodiments, at least one R^{N2ab} is —OH, —NH₂, —C(═O)H, or —C(═O)OH.

In some embodiments, at least one R^{N2ab} is —OH.

In some embodiments, at least one R^{N2ab} is —NH₂.

In some embodiments, at least one R^{N2ab} is —C(═O)H.

In some embodiments, at least one R^{N2ab} is —C(═O)OH.

In some embodiments, at least one R^{N2ab} is —O(C₁-C₆ alkyl), —NH(C₁-C₆ alkyl), —N(C₁-C₆ alkyl)₂, —C(═O)(C₁-C₆ alkyl), —C(═O)O(C₁-C₆ alkyl), or —NHC(═O)O(C₁-C₆ alkyl).

In some embodiments, at least one R^{N2ab} is —O(C₁-C₆ alkyl).

In some embodiments, at least one R^{N2ab} is —NH(C₁-C₆ alkyl) or —N(C₁-C₆ alkyl)₂.

In some embodiments, at least one R^{N2ab} is —C(═O)(C₁-C₆ alkyl).

In some embodiments, at least one R^{N2ab} is —C(═O)O(C₁-C₆ alkyl) (e.g., —C(═O)O(ethyl)).

In some embodiments, at least one R^{N2ab} is —NHC(═O)O(C₁-C₆ alkyl).

In some embodiments, at least one R^{N2ab} is —S(═O)₂(C₁-C₆ alkyl) or —S(═O)₂N(C₁-C₆ alkyl)₂.

In some embodiments, at least one R^{N2ab} is —S(═O)₂(C₁-C₆ alkyl).

In some embodiments, at least one R^{N2ab} is —S(═O)₂N(C₁-C₆ alkyl)₂.

### Exemplary Embodiments of the Compounds

In some embodiments, the compound is of Formula (II):

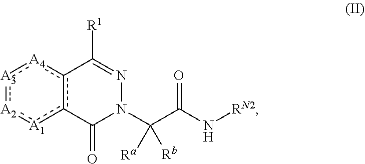

or a prodrug, solvate, or pharmaceutically acceptable salt thereof.

In some embodiments, the compound is of Formula (II-a):

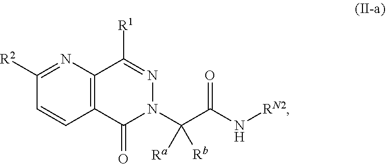

or a prodrug, solvate, or pharmaceutically acceptable salt thereof.

In some embodiments, the compound is of Formula (III-a), (III-b), (III-c), (III-d), (III-e), (III-f), or (III-g):

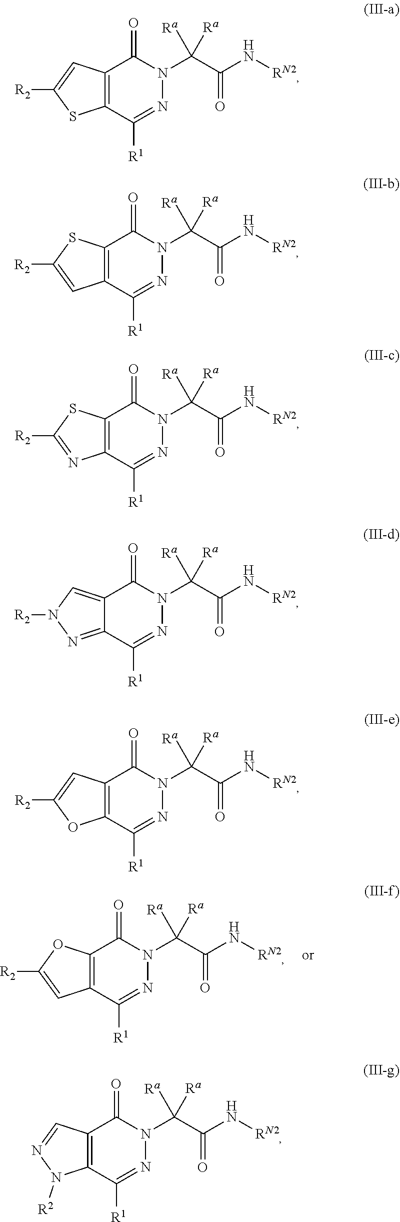

or a prodrug, solvate, or pharmaceutically acceptable salt thereof.

In some embodiments, the compound of Formula (III) is selected from Formula (III-b), Formula (III-d), and Formula (III-e).

In some embodiments, the compound of Formula (III) is a compound of Formula (III-e).

In some embodiments, the compound is selected from the compounds described in Table 1 and prodrugs and pharmaceutically acceptable salts thereof.

In some embodiments, the compound is selected from the compounds described in Table 1 and pharmaceutically acceptable salts thereof.

In some embodiments, the compound of Formula (III) is selected from the compounds described in Table 1.

TABLE 1

| Compound | |
|---|---|
| No | Structure |
| 1 | 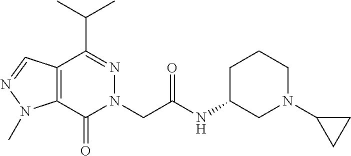 [SMILES: CC(C)c1nn(CC(=O)N[C@@H]2CCCN(C3CC3)C2)c(=O)c2c1cnn2C] |
| 2 | 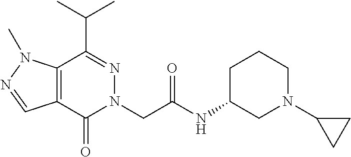 [SMILES: CC(C)c1nn(CC(=O)N[C@@H]2CCCN(C3CC3)C2)c(=O)c2cnn(C)c12] |
| 3 | 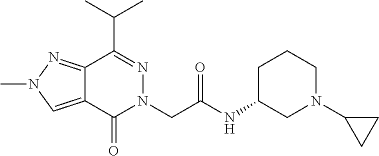 [SMILES: CC(C)c1nn(CC(=O)N[C@@H]2CCCN(C3CC3)C2)c(=O)c2cn(C)nc12] |
| 4 | 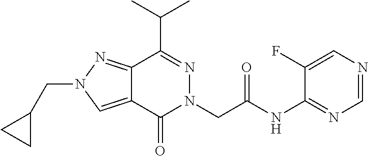 |
| 5 | 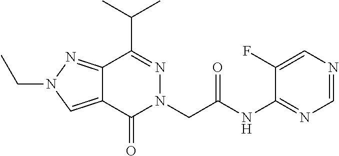 [SMILES: CCn1cc2c(=O)n(CC(=O)Nc3ncncc3F)nc(C(C)C)c2n1] |
| 6 | 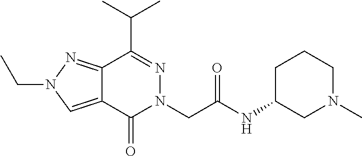 [SMILES: CCn1cc2c(=O)n(CC(=O)N[C@@H]3CCCN(C)C3)nc(C(C)C)c2n1] |
| 7 | 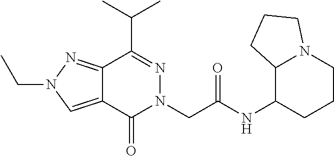 |
| 8 | 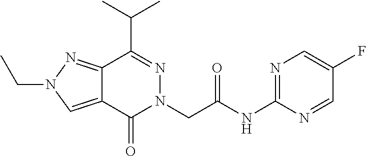 |
| 9 | 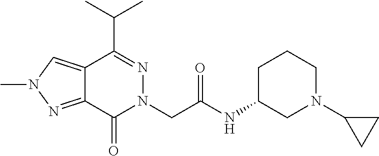 [SMILES: CC(C)c1nn(CC(=O)N[C@@H]2CCCN(C3CC3)C2)c(=O)c2nn(C)cc12] |
| 10 | 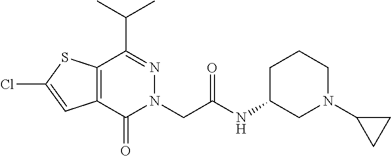 [SMILES: CC(C)c1nn(CC(=O)N[C@@H]2CCCN(C3CC3)C2)c(=O)c2cc(Cl)sc12] |
| 11 | 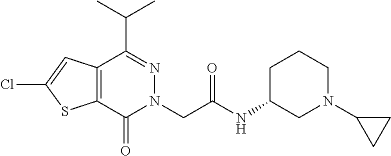 [SMILES: CC(C)c1nn(CC(=O)N[C@@H]2CCCN(C3CC3)C2)c(=O)c2sc(Cl)cc12] |
| 12 | 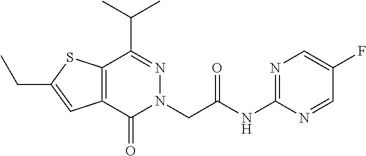 [SMILES: CCc1cc2c(=O)n(CC(=O)Nc3ncc(F)cn3)nc(C(C)C)c2s1] |
| 13 | 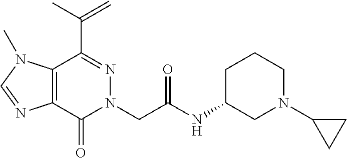 [SMILES: C=C(C)c1nn(CC(=O)N[C@@H]2CCCN(C3CC3)C2)c(=O)c2ncn(C)c12] |
| 14 | 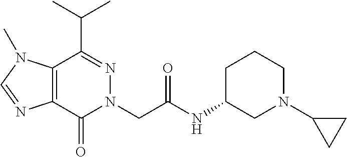 [SMILES: CC(C)c1nn(CC(=O)N[C@@H]2CCCN(C3CC3)C2)c(=O)c2ncn(C)c12] |
| 15 | 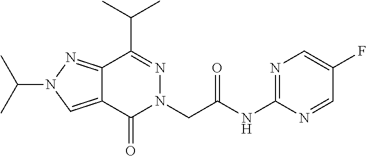 [SMILES: CC(C)c1nn(CC(=O)Nc2ncc(F)cn2)c(=O)c2cn(C(C)C)nc12] |
| 16 | 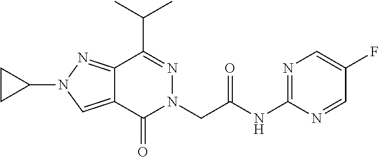 |
| 17 | 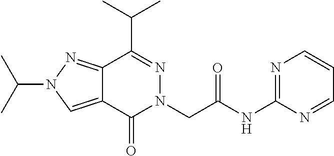 |
| 20 | 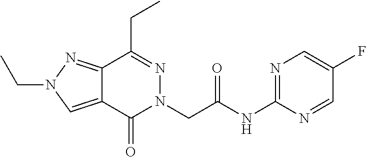 [SMILES: CCc1nn(CC(=O)Nc2ncc(F)cn2)c(=O)c2cn(CC)nc12] |
| 21 | 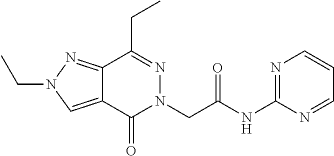 |
| 22 | 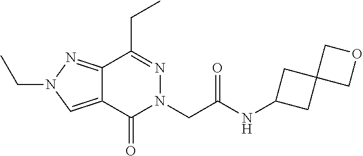 [SMILES: CCc1nn(CC(=O)NC2CC3(COC3)C2)c(=O)c2cn(CC)nc12] |
| 23 | 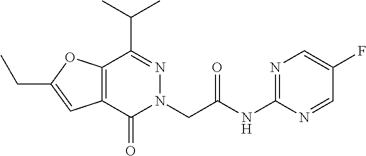 [SMILES: CCc1cc2c(=O)n(CC(=O)Nc3ncc(F)cn3)nc(C(C)C)c2o1] |
| 24 | 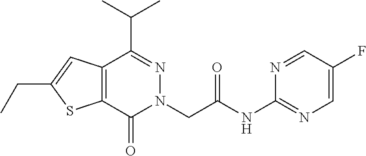 [SMILES: CCc1cc2c(C(C)C)nn(CC(=O)Nc3ncc(F)cn3)c(=O)c2s1] |
| 29 | 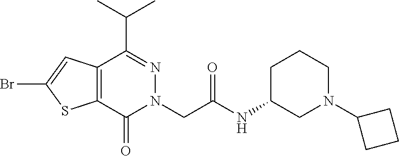 [SMILES: CC(C)c1nn(CC(=O)N[C@@H]2CCCN(C3CCC3)C2)c(=O)c2sc(Br)cc12] |
| 30 | 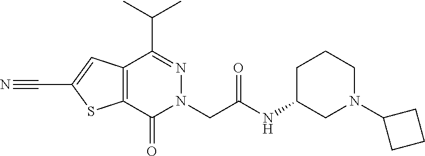 [SMILES: CC(C)c1nn(CC(=O)N[C@@H]2CCCN(C3CCC3)C2)c(=O)c2sc(C#N)cc12] |
| 31 | 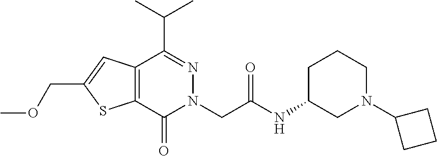 [SMILES: COCc1cc2c(C(C)C)nn(CC(=O)N[C@@H]3CCCN(C4CCC4)C3)c(=O)c2s1] |
| 39 | 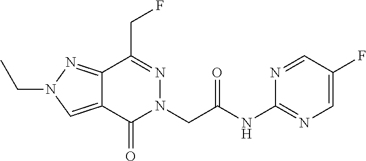 |
| 40 | 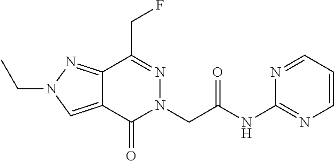 [SMILES: CCn1cc2c(=O)n(CC(=O)Nc3ncccn3)nc(CF)c2n1] |
| 50 | 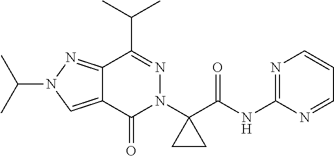 |
| 51 | 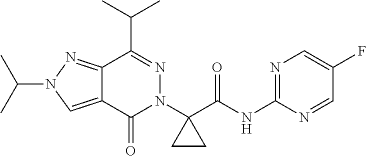 [SMILES: CC(C)c1nn(C2(C(=O)Nc3ncc(F)cn3)CC2)c(=O)c2cn(C(C)C)nc12] |
| 52 | 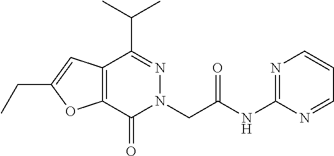 [SMILES: CCc1cc2c(C(C)C)nn(CC(=O)Nc3ncccn3)c(=O)c2o1] |
| 53 | 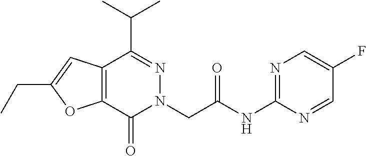 [SMILES: CCc1cc2c(C(C)C)nn(CC(=O)Nc3ncc(F)cn3)c(=O)c2o1] |
| 54 | 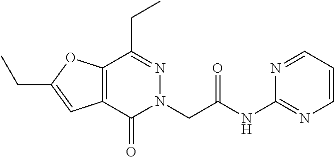 [SMILES: CCc1cc2c(=O)n(CC(=O)Nc3ncccn3)nc(CC)c2o1] |
| 55 | 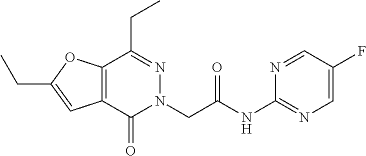 [SMILES: CCc1cc2c(=O)n(CC(=O)Nc3ncc(F)cn3)nc(CC)c2o1] |
| 56 | 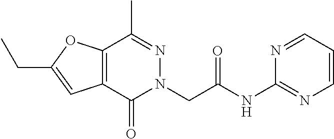 [SMILES: CCc1cc2c(=O)n(CC(=O)Nc3ncccn3)nc(C)c2o1] |
| 57 | 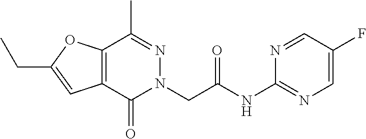 [SMILES: CCc1cc2c(=O)n(CC(=O)Nc3ncc(F)cn3)nc(C)c2o1] |
| 58 | 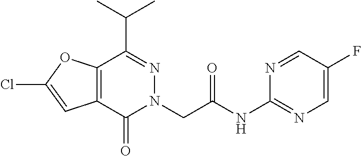 [SMILES: CC(C)c1nn(CC(=O)Nc2ncc(F)cn2)c(=O)c2cc(Cl)oc12] |
| 59 | 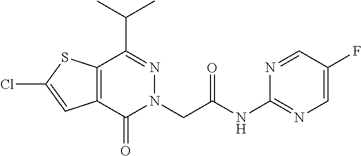 [SMILES: CC(C)c1nn(CC(=O)Nc2ncc(F)cn2)c(=O)c2cc(Cl)sc12] |
| 60 | 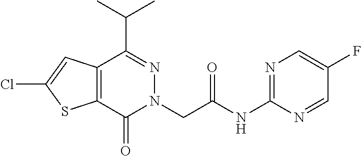 [SMILES: CC(C)c1nn(CC(=O)Nc2ncc(F)cn2)c(=O)c2sc(Cl)cc12] |
| 62 | 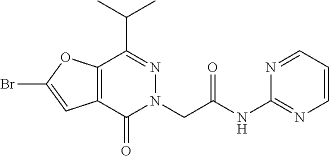 [SMILES: CC(C)c1nn(CC(=O)Nc2ncccn2)c(=O)c2cc(Br)oc12] |
| 63 | 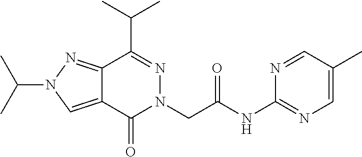 [SMILES: Cc1cnc(NC(=O)Cn2nc(C(C)C)c3nn(C(C)C)cc3c2=O)nc1] |
| 64 | 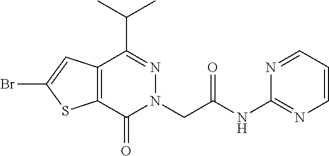 [SMILES: CC(C)c1nn(CC(=O)Nc2ncccn2)c(=O)c2sc(Br)cc12] |
| 65 | 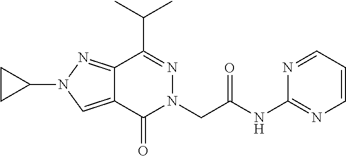 [SMILES: CC(C)c1nn(CC(=O)Nc2ncccn2)c(=O)c2cn(C3CC3)nc12] |
| 66 | 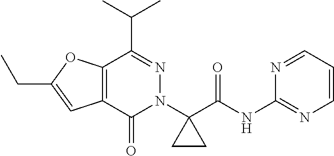 [SMILES: CCc1cc2c(=O)n(C3(C(=O)Nc4ncccn4)CC3)nc(C(C)C)c2o1] |
| 67 | 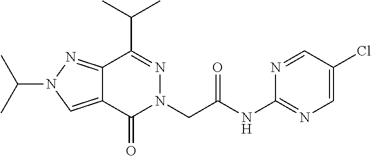 [SMILES: CC(C)c1nn(CC(=O)Nc2ncc(Cl)cn2)c(=O)c2cn(C(C)C)nc12] |
| 68 | 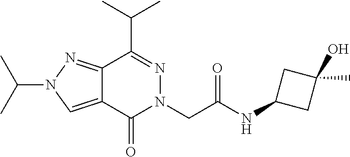 [SMILES: CC(C)c1nn(CC(=O)N[C@H]2C[C@@](C)(O)C2)c(=O)c2cn(C(C)C)nc12] |
| 69 | 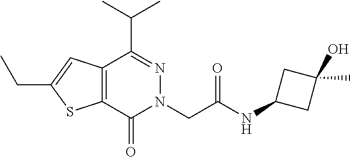 [SMILES: CCc1cc2c(C(C)C)nn(CC(=O)N[C@H]3C[C@@](C)(O)C3)c(=O)c2s1] |
| 70 | 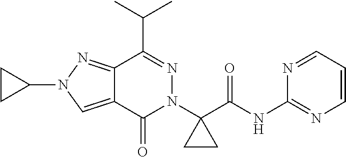 [SMILES: CC(C)c1nn(C2(C(=O)Nc3ncccn3)CC2)c(=O)c2cn(C3CC3)nc12] |
| 71 | 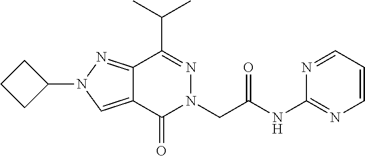 [SMILES: CC(C)c1nn(CC(=O)Nc2ncccn2)c(=O)c2cn(C3CCC3)nc12] |
| 72 | 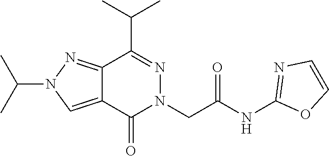 [SMILES: CC(C)c1nn(CC(=O)Nc2ncco2)c(=O)c2cn(C(C)C)nc12] |
| 73 | 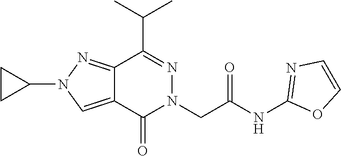 [SMILES: CC(C)c1nn(CC(=O)Nc2ncco2)c(=O)c2cn(C3CC3)nc12] |
| 74 | 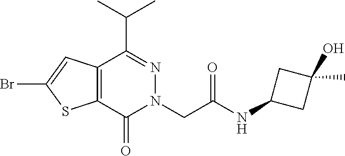 [SMILES: CC(C)c1nn(CC(=O)N[C@H]2C[C@@](C)(O)C2)c(=O)c2sc(Br)cc12] |
| 75 | 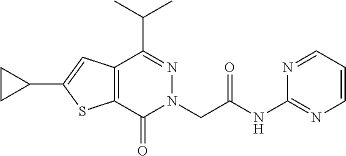 [SMILES: CC(C)c1nn(CC(=O)Nc2ncccn2)c(=O)c2sc(C3CC3)cc12] |
| 76 | 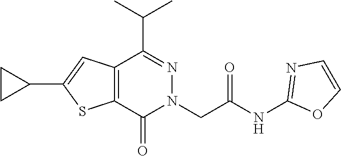 [SMILES: CC(C)c1nn(CC(=O)Nc2ncco2)c(=O)c2sc(C3CC3)cc12] |
| 77 | 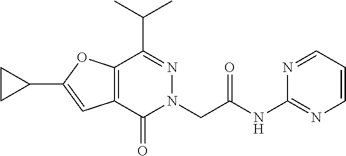 [SMILES: CC(C)c1nn(CC(=O)Nc2ncccn2)c(=O)c2cc(C3CC3)oc12] |
| 78 | 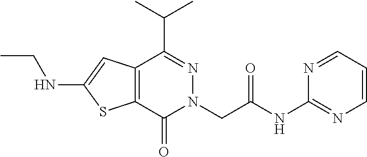 [SMILES: CCNc1cc2c(C(C)C)nn(CC(=O)Nc3ncccn3)c(=O)c2s1] |
| 79 | 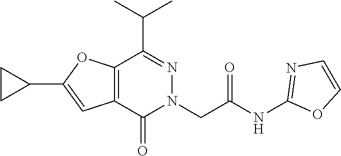 [SMILES: CC(C)c1nn(CC(=O)Nc2ncco2)c(=O)c2cc(C3CC3)oc12] |
| 80 | 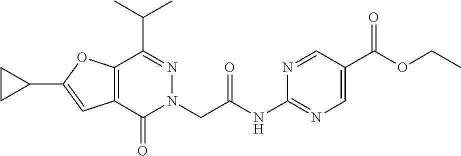 [SMILES: CCOC(=O)c1cnc(NC(=O)Cn2nc(C(C)C)c3oc(C4CC4)cc3c2=O)nc1] |
| 81 | 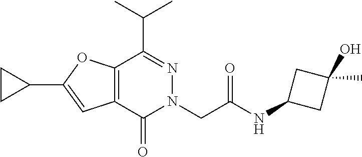 [SMILES: CC(C)c1nn(CC(=O)N[C@H]2C[C@@](C)(O)C2)c(=O)c2cc(C3CC3)oc12] |
| 82 | 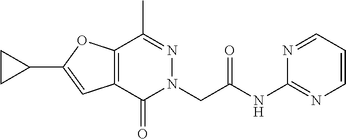 [SMILES: Cc1nn(CC(=O)Nc2ncccn2)c(=O)c2cc(C3CC3)oc12] |
| 83 | 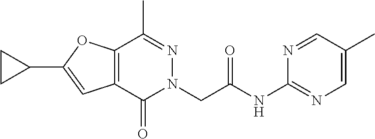 [SMILES: Cc1cnc(NC(=O)Cn2nc(C)c3oc(C4CC4)cc3c2=O)nc1] |
| 84 | 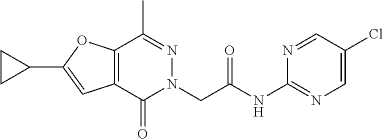 [SMILES: Cc1nn(CC(=O)Nc2ncc(Cl)cn2)c(=O)c2cc(C3CC3)oc12] |
| 85 | 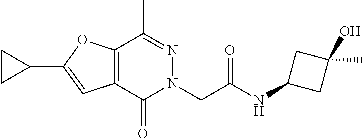 [SMILES: Cc1nn(CC(=O)N[C@H]2C[C@@](C)(O)C2)c(=O)c2cc(C3CC3)oc12] |
| 86 | 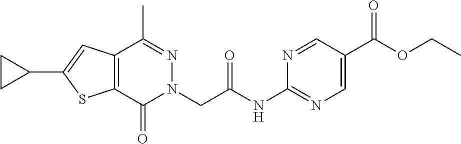 [SMILES: CCOC(=O)c1cnc(NC(=O)Cn2nc(C)c3cc(C4CC4)sc3c2=O)nc1] |
| 87 | 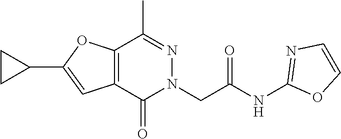 [SMILES: Cc1nn(CC(=O)Nc2ncco2)c(=O)c2cc(C3CC3)oc12] |
| 88 | 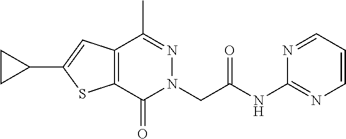 [SMILES: Cc1nn(CC(=O)Nc2ncccn2)c(=O)c2sc(C3CC3)cc12] |
| 89 | 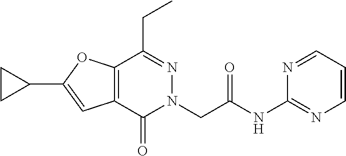 [SMILES: CCc1nn(CC(=O)Nc2ncccn2)c(=O)c2cc(C3CC3)oc12] |
| 90 | 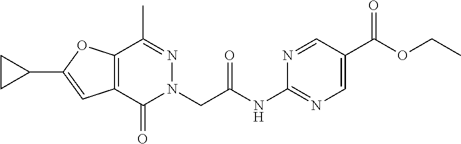 [SMILES: CCOC(=O)c1cnc(NC(=O)Cn2nc(C)c3oc(C4CC4)cc3c2=O)nc1] |
| 91 | 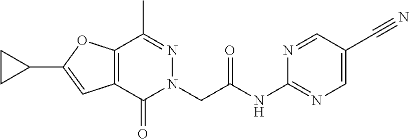 [SMILES: Cc1nn(CC(=O)Nc2ncc(C#N)cn2)c(=O)c2cc(C3CC3)oc12] |
| 92 | 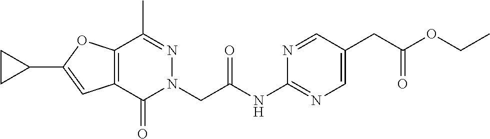 [SMILES: CCOC(=O)Cc1cnc(NC(=O)Cn2nc(C)c3oc(C4CC4)cc3c2=O)nc1] |
| 93 | 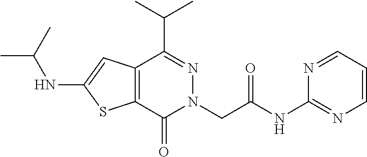 [SMILES: CC(C)Nc1cc2c(C(C)C)nn(CC(=O)Nc3ncccn3)c(=O)c2s1] |
| 94 | 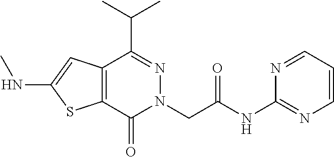 [SMILES: CNc1cc2c(C(C)C)nn(CC(=O)Nc3ncccn3)c(=O)c2s1] |
| 95 | 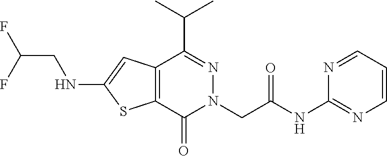 [SMILES: CC(C)c1nn(CC(=O)Nc2ncccn2)c(=O)c2sc(NCC(F)F)cc12] |
| 96 | 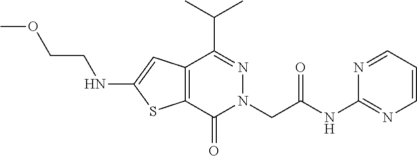 [SMILES: COCCNc1cc2c(C(C)C)nn(CC(=O)Nc3ncccn3)c(=O)c2s1] |
| 97 | 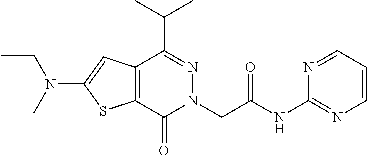 [SMILES: CCN(C)c1cc2c(C(C)C)nn(CC(=O)Nc3ncccn3)c(=O)c2s1] |
| 98 | 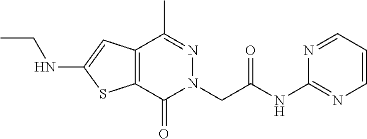 [SMILES: CCNc1cc2c(C)nn(CC(=O)Nc3ncccn3)c(=O)c2s1] |
| 99 | 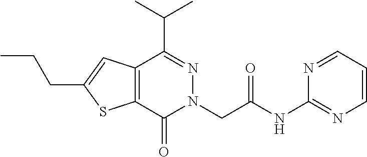 [SMILES: CCCc1cc2c(C(C)C)nn(CC(=O)Nc3ncccn3)c(=O)c2s1] |
| 100 | 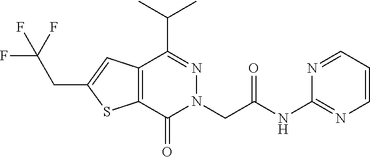 [SMILES: CC(C)c1nn(CC(=O)Nc2ncccn2)c(=O)c2sc(CC(F)(F)F)cc12] |
| 101 | 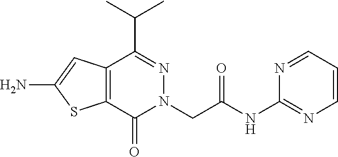 [SMILES: CC(C)c1nn(CC(=O)Nc2ncccn2)c(=O)c2sc(N)cc12] |
| 102 | 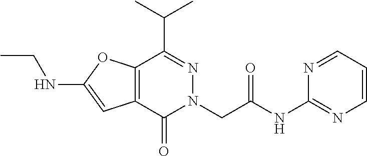 [SMILES: CCNc1cc2c(=O)n(CC(=O)Nc3ncccn3)nc(C(C)C)c2o1] |
| 103 | 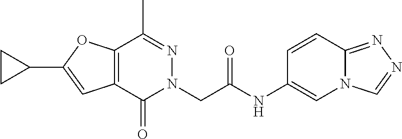 [SMILES: Cc1nn(CC(=O)Nc2ccc3nncn3c2)c(=O)c2cc(C3CC3)oc12] |
| 104 | 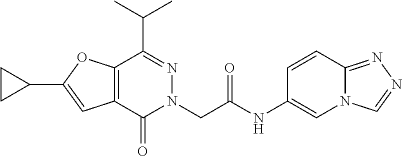 [SMILES: CC(C)c1nn(CC(=O)Nc2ccc3nncn3c2)c(=O)c2cc(C3CC3)oc12] |
| 105 | 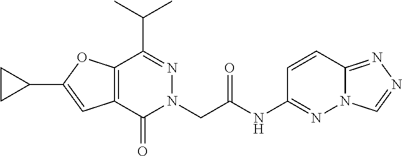 [SMILES: CC(C)c1nn(CC(=O)Nc2ccc3nncn3n2)c(=O)c2cc(C3CC3)oc12] |
| 106 | 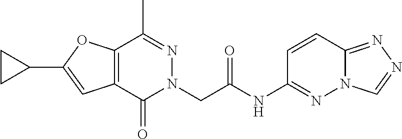 [SMILES: Cc1nn(CC(=O)Nc2ccc3nncn3n2)c(=O)c2cc(C3CC3)oc12] |
| 107 | 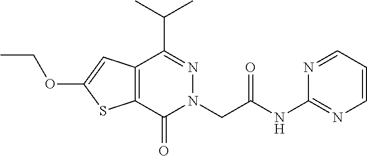 [SMILES: CCOc1cc2c(C(C)C)nn(CC(=O)Nc3ncccn3)c(=O)c2s1] |
| 108 | 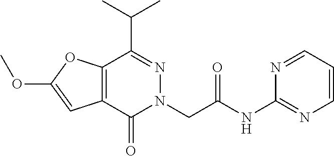 [SMILES: COc1cc2c(=O)n(CC(=O)Nc3ncccn3)nc(C(C)C)c2o1] |
| 109 | 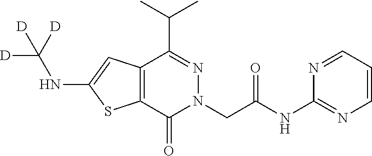 [SMILES: [2H]C([2H])([2H])Nc1cc2c(C(C)C)nn(CC(=O)Nc3ncccn3)c(=O)c2s1] |
| 110 | 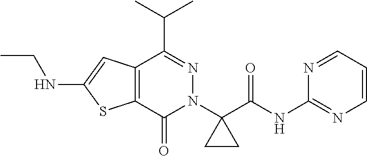 [SMILES: CCNc1cc2c(C(C)C)nn(C3(C(=O)Nc4ncccn4)CC3)c(=O)c2s1] |
| 111 | 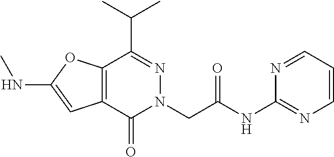 [SMILES: CNc1cc2c(=O)n(CC(=O)Nc3ncccn3)nc(C(C)C)c2o1] |
| 112 | 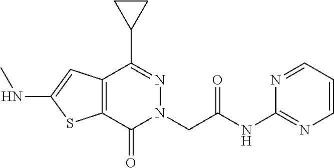 [SMILES: CNc1cc2c(C3CC3)nn(CC(=O)Nc3ncccn3)c(=O)c2s1] |
| 113 | 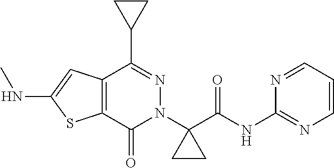 [SMILES: CNc1cc2c(C3CC3)nn(C3(C(=O)Nc4ncccn4)CC3)c(=O)c2s1] |
| 114 | 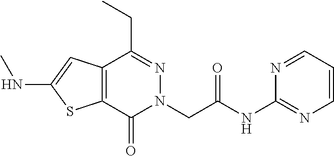 [SMILES: CCc1nn(CC(=O)Nc2ncccn2)c(=O)c2sc(NC)cc12] |
| 115 | 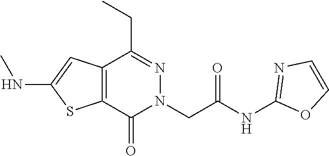 [SMILES: CCc1nn(CC(=O)Nc2ncco2)c(=O)c2sc(NC)cc12] |
| 116 | 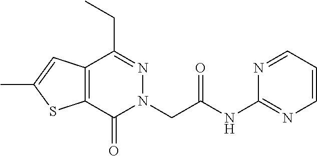 [SMILES: CCc1nn(CC(=O)Nc2ncccn2)c(=O)c2sc(C)cc12] |
| 117 | 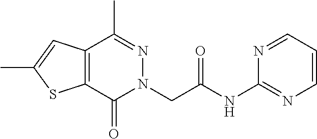 [SMILES: Cc1cc2c(C)nn(CC(=O)Nc3ncccn3)c(=O)c2s1] |
| 118 | 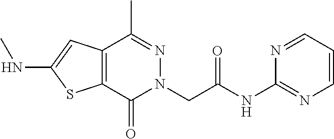 [SMILES: CNc1cc2c(C)nn(CC(=O)Nc3ncccn3)c(=O)c2s1] |
| 119 | 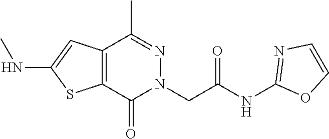 [SMILES: CNc1cc2c(C)nn(CC(=O)Nc3ncco3)c(=O)c2s1] |
| 120 | 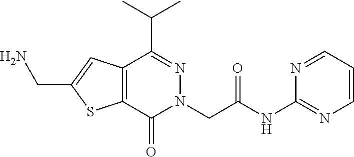 [SMILES: CC(C)c1nn(CC(=O)Nc2ncccn2)c(=O)c2sc(CN)cc12] |

In some embodiments, the compound of Formula (I) is selected from the compounds described in Table 2.

TABLE 2

| Com- | |
|---|---|
| pound | |
| No | Structure |
| 18 | 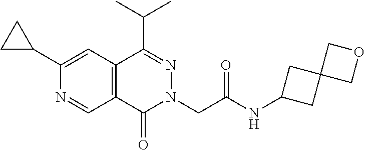 [SMILES: CC(C)c1nn(CC(=O)NC2CC3(COC3)C2)c(=O)c2cnc(C3CC3)cc12] |
| 19 | 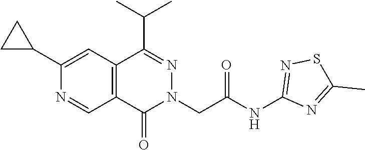 [SMILES: Cc1nc(NC(=O)Cn2nc(C(C)C)c3cc(C4CC4)ncc3c2=O)ns1] |
| 25 | 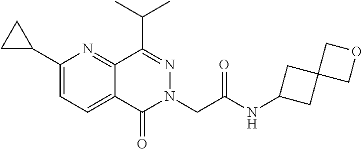 [SMILES: CC(C)c1nn(CC(=O)NC2CC3(COC3)C2)c(=O)c2ccc(C3CC3)nc12] |
| 26 | 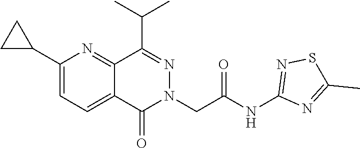 [SMILES: Cc1nc(NC(=O)Cn2nc(C(C)C)c3nc(C4CC4)ccc3c2=O)ns1] |
| 27 | 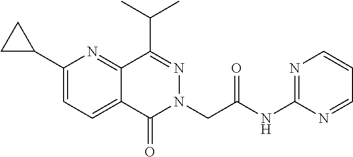 [SMILES: CC(C)c1nn(CC(=O)Nc2ncccn2)c(=O)c2ccc(C3CC3)nc12] |
| 28 | 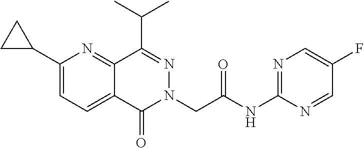 [SMILES: CC(C)c1nn(CC(=O)Nc2ncc(F)cn2)c(=O)c2ccc(C3CC3)nc12] |
| 32 | 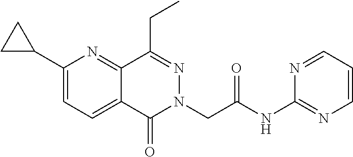 [SMILES: CCc1nn(CC(=O)Nc2ncccn2)c(=O)c2ccc(C3CC3)nc12] |
| 33 | 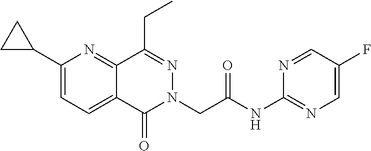 [SMILES: CCc1nn(CC(=O)Nc2ncc(F)cn2)c(=O)c2ccc(C3CC3)nc12] |
| 34 | 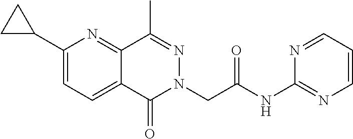 [SMILES: Cc1nn(CC(=O)Nc2ncccn2)c(=O)c2ccc(C3CC3)nc12] |
| 35 | 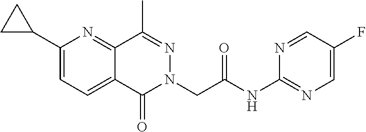 [SMILES: Cc1nn(CC(=O)Nc2ncc(F)cn2)c(=O)c2ccc(C3CC3)nc12] |
| 36 | 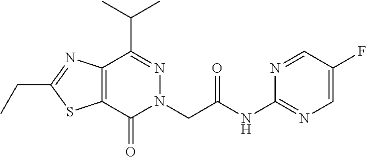 [SMILES: CCc1nc2c(C(C)C)nn(CC(=O)Nc3ncc(F)cn3)c(=O)c2s1] |
| 37 | 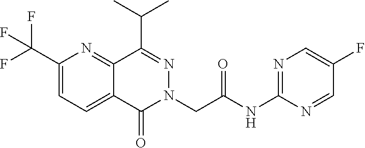 [SMILES: CC(C)c1nn(CC(=O)Nc2ncc(F)cn2)c(=O)c2ccc(C(F)(F)F)nc12] |
| 38 | 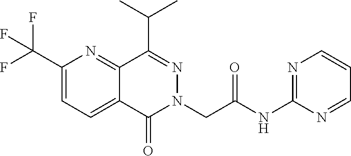 [SMILES: CC(C)c1nn(CC(=O)Nc2ncccn2)c(=O)c2ccc(C(F)(F)F)nc12] |
| 42 | 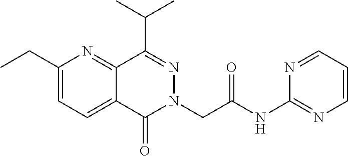 [SMILES: CCc1ccc2c(=O)n(CC(=O)Nc3ncccn3)nc(C(C)C)c2n1] |
| 43 | 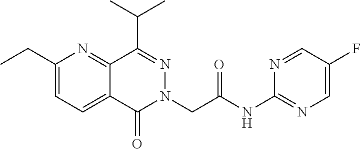 [SMILES: CCc1ccc2c(=O)n(CC(=O)Nc3ncc(F)cn3)nc(C(C)C)c2n1] |
| 44 | 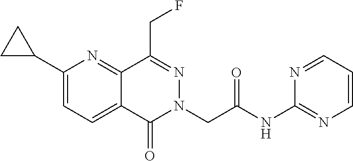 [SMILES: O=C(Cn1nc(CF)c2nc(C3CC3)ccc2c1=O)Nc1ncccn1] |
| 45 | 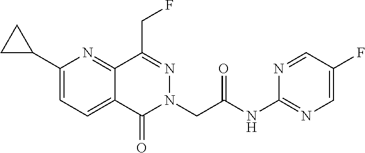 [SMILES: O=C(Cn1nc(CF)c2nc(C3CC3)ccc2c1=O)Nc1ncc(F)cn1] |
| 46 | 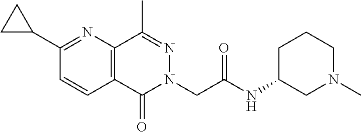 [SMILES: Cc1nn(CC(=O)N[C@@H]2CCCN(C)C2)c(=O)c2ccc(C3CC3)nc12] |
| 47 | 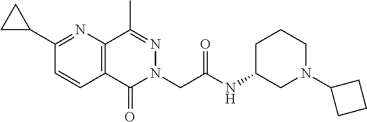 [SMILES: Cc1nn(CC(=O)N[C@@H]2CCCN(C3CCC3)C2)c(=O)c2ccc(C3CC3)nc12] |
| 48 | 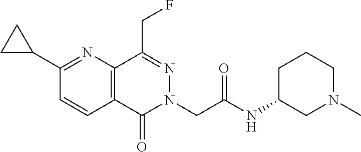 [SMILES: CN1CCC[C@@H](NC(=O)Cn2nc(CF)c3nc(C4CC4)ccc3c2=O)C1] |
| 49 | 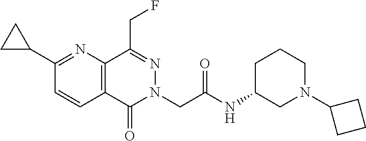 [SMILES: O=C(Cn1nc(CF)c2nc(C3CC3)ccc2c1=O)N[C@@H]1CCCN(C2CCC2)C1] |
| 61 | 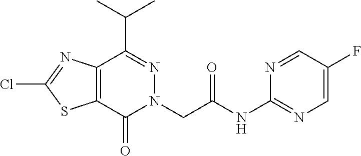 [SMILES: CC(C)c1nn(CC(=O)Nc2ncc(F)cn2)c(=O)c2sc(Cl)nc12] |

In some aspects, the present disclosure provides a compound being an isotopic derivative (e.g., isotopically labeled compound) of any one of the compounds of the Formulae disclosed herein.

In some embodiments, the compound is a protonated compound of any one of the Formulae disclosed herein and pharmaceutically acceptable salts thereof.

In some embodiments, the compound is a protonated compound of any one of the Formulae disclosed herein.

In some embodiments, the compound is a protonated compound of any one of the compounds described in Table 1 and pharmaceutically acceptable salts thereof.

In some embodiments, the compound is a protonated compound of any one of the compounds described in Table 1.

In some embodiments, the compound is an isotopic derivative of any one of the compounds described in Table 1 and prodrugs and pharmaceutically acceptable salts thereof.

In some embodiments, the compound is an isotopic derivative of any one of the compounds described in Table 1 and pharmaceutically acceptable salts thereof.

In some embodiments, the compound is an isotopic derivative of any one of the compounds described in Table 1.

It is understood that the isotopic derivative can be prepared using any of a variety of art-recognised techniques. For example, the isotopic derivative can generally be prepared by carrying out the procedures disclosed in the Schemes and/or in the Examples described herein, by substituting an isotopically labeled reagent for a non-isotopically labeled reagent.

In some embodiments, the isotopic derivative is a deuterium labeled compound.

In some embodiments, the isotopic derivative is a deuterium labeled compound of any one of the compounds of the Formulae disclosed herein.

In some embodiments, the compound is a deuterium labeled compound of any one of the compounds described in Table 1 and prodrugs and pharmaceutically acceptable salts thereof.

In some embodiments, the compound is a deuterium labeled compound of any one of the compounds described in Table 1 and pharmaceutically acceptable salts thereof.

In some embodiments, the compound is a deuterium labeled compound of any one of the compounds described in Table 1.

It is understood that the deuterium labeled compound comprises a deuterium atom having an abundance of deuterium that is substantially greater than the natural abundance of deuterium, which is 0.015%.

In some embodiments, the deuterium labeled compound has a deuterium enrichment factor for each deuterium atom of at least 3500 (52.5% deuterium incorporation at each deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). As used herein, the term “deuterium enrichment factor” means the ratio between the deuterium abundance and the natural abundance of a deuterium.

It is understood that the deuterium labeled compound can be prepared using any of a variety of art-recognised techniques. For example, the deuterium labeled compound can generally be prepared by carrying out the procedures disclosed in the Schemes and/or in the Examples described herein, by substituting a deuterium labeled reagent for a non-deuterium labeled reagent.

A compound of the invention or a pharmaceutically acceptable salt or solvate thereof that contains the aforementioned deuterium atom(s) is within the scope of the invention. Further, substitution with deuterium (i.e., ²H) may afford certain therapeutic advantages resulting from greater metabolic stability, e.g., increased in vivo half-life or reduced dosage requirements.

For the avoidance of doubt it is to be understood that, where in this specification a group is qualified by “described herein”, the said group encompasses the first occurring and broadest definition as well as each and all of the particular definitions for that group.

A suitable pharmaceutically acceptable salt of a compound of the disclosure is, for example, an acid-addition salt of a compound of the disclosure, which is sufficiently basic, for example, an acid-addition salt with, for example, an inorganic or organic acid, for example hydrochloric, hydrobromic, sulphuric, phosphoric, formic, citric methane sulphonate or maleic acid. In addition, a suitable pharmaceutically acceptable salt of a compound of the disclosure which is sufficiently acidic is an alkali metal salt, for example a sodium or potassium salt, an alkaline earth metal salt, for example a calcium or magnesium salt, an ammonium salt or a salt with an organic base which affords a pharmaceutically acceptable cation, for example a salt with methylamine, dimethylamine, diethylamine, trimethylamine, piperidine, morpholine or tris-(2-hydroxyethyl)amine.

It will be understood that the compounds of any one of the Formulae disclosed herein and any pharmaceutically acceptable salts thereof, comprise stereoisomers, mixtures of stereoisomers, polymorphs of all isomeric forms of said compounds.

As used herein, the term “isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

As used herein, the term “chiral centre” refers to a carbon atom bonded to four nonidentical substituents.

As used herein, the term “chiral isomer” means a compound with at least one chiral centre. Compounds with more than one chiral centre may exist either as an individual diastereomer or as a mixture of diastereomers, termed “diastereomeric mixture.” When one chiral centre is present, a stereoisomer may be characterised by the absolute configuration (R or S) of that chiral centre. Absolute configuration refers to the arrangement in space of the substituents attached to the chiral centre. The substituents attached to the chiral centre under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

As used herein, the term “geometric isomer” means the diastereomers that owe their existence to hindered rotation about double bonds or a cycloalkyl linker (e.g., 1,3-cyclobutyl). These configurations are differentiated in their names by the prefixes cis and trans, or Z and E, which indicate that the groups are on the same or opposite side of the double bond in the molecule according to the Cahn-Ingold-Prelog rules.

It is to be understood that the compounds of the present disclosure may be depicted as different chiral isomers or geometric isomers. It is also to be understood that when compounds have chiral isomeric or geometric isomeric forms, all isomeric forms are intended to be included in the scope of the present disclosure, and the naming of the compounds does not exclude any isomeric forms, it being understood that not all isomers may have the same level of activity.

It is to be understood that the structures and other compounds discussed in this disclosure include all atropic isomers thereof. It is also to be understood that not all atropic isomers may have the same level of activity.

As used herein, the term “atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

As used herein, the term “tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerisation is possible, a chemical equilibrium of the tautomers will be reached. The exact ratio of the tautomers depends on several factors, including temperature, solvent and pH. The concept of tautomers that are interconvertible by tautomerisations is called tautomerism. Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

It is to be understood that the compounds of the present disclosure may be depicted as different tautomers. It should also be understood that when compounds have tautomeric forms, all tautomeric forms are intended to be included in the scope of the present disclosure, and the naming of the compounds does not exclude any tautomer form. It will be understood that certain tautomers may have a higher level of activity than others.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric centre, for example, it is bonded to four different groups, a pair of enantiomers is possible. An enantiomer can be characterised by the absolute configuration of its asymmetric centre and is described by the R- and S-sequencing rules of Cahn and Prelog, or by the manner in which the molecule rotates the plane of polarised light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this disclosure may possess one or more asymmetric centres; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the disclosure may have geometric isomeric centres (E- and Z-isomers). It is to be understood that the present disclosure encompasses all optical, diastereoisomers and geometric isomers and mixtures thereof that possess inflammasome inhibitory activity.

The present disclosure also encompasses compounds of the disclosure as defined herein which comprise one or more isotopic substitutions.

It is to be understood that the compounds of any Formula described herein include the compounds themselves, as well as their salts, and their solvates, if applicable. A salt, for example, can be formed between an anion and a positively charged group (e.g., amino) on a substituted compound disclosed herein. Suitable anions include chloride, bromide, iodide, sulphate, bisulphate, sulphamate, nitrate, phosphate, citrate, methanesulphonate, glutamate, glucuronate, glutarate, malate, maleate, succinate, fumarate, tartrate, tosylate, salicylate, lactate, naphthalenesulphonate, and acetate.

The compound of any one of the Formulae described herein may be protonated at a physiological pH. Thus, a compound may have a positive or partial positive charge at physiological pH. Such compounds may be referred to as cationic or ionizable compounds. The compound of any one of the Formulae described herein may also be zwitterionic, i.e., neutral molecules having both a positive and a negative charge.

As used herein, the term “pharmaceutically acceptable anion” refers to an anion suitable for forming a pharmaceutically acceptable salt. Likewise, a salt can also be formed between a cation and a negatively charged group (e.g., carboxylate) on a substituted compound disclosed herein. Suitable cations include sodium ion, potassium ion, magnesium ion, calcium ion, and an ammonium cation such as tetramethylammonium ion or diethylamine ion. The substituted compounds disclosed herein also include those salts containing quaternary nitrogen atoms.

It is to be understood that the compounds of the present disclosure, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

As used herein, the term “solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

As used herein, the term “derivative” refers to compounds that have a common core structure and are substituted with various groups as described herein.

As used herein, the term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include, but are not limited to, acyl sulphonamides, tetrazoles, sulphonates and phosphonates. See, e.g., Patani and LaVoie, Chem. Rev. 96, 3147-3176, 1996.

It is also to be understood that certain compounds of any one of the Formulae disclosed herein may exist in solvated as well as unsolvated forms such as, for example, hydrated forms. A suitable pharmaceutically acceptable solvate is, for example, a hydrate such as hemi-hydrate, a mono-hydrate, a di-hydrate or a tri-hydrate. It is to be understood that the disclosure encompasses all such solvated forms that possess inflammasome inhibitory activity.

It is also to be understood that certain compounds of any one of the Formulae disclosed herein may exhibit polymorphism, and that the disclosure encompasses all such forms, or mixtures thereof, which possess inflammasome inhibitory activity. It is generally known that crystalline materials may be analysed using conventional techniques such as X-Ray Powder Diffraction analysis, Differential Scanning Calorimetry, Thermal Gravimetric Analysis, Diffuse Reflectance Infrared Fourier Transform (DRIFT) spectroscopy, Near Infrared (NIR) spectroscopy, solution and/or solid state nuclear magnetic resonance spectroscopy. The water content of such crystalline materials may be determined by Karl Fischer analysis.

Compounds of any one of the Formulae disclosed herein may exist in a number of different tautomeric forms and references to compounds of Formula (III) include all such forms. For the avoidance of doubt, where a compound can exist in one of several tautomeric forms, and only one is specifically described or shown, all others are nevertheless embraced by Formula (III). Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

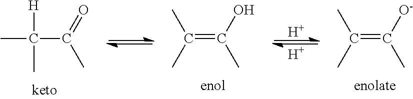

Compounds of any one of the Formulae disclosed herein containing an amine function may also form N-oxides. A reference herein to a compound of Formula (III) that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-oxides can be formed by treatment of the corresponding amine with an oxidising agent such as hydrogen peroxide or a peracid (e.g. a peroxycarboxylic acid), see for example Advanced Organic Chemistry, by Jerry March, 4th Edition, Wiley Interscience, pages. More particularly, N-oxides can be made by the procedure of L. W. Deady (Syn. Comm. 1977, 7, 509-514) in which the amine compound is reacted with meta-chloroperoxybenzoic acid (mCPBA), for example, in an inert solvent such as dichloromethane.

The compounds of any one of the Formulae disclosed herein may be administered in the form of a prodrug which is broken down in the human or animal body to release a compound of the disclosure. A prodrug may be used to alter the physical properties and/or the pharmacokinetic properties of a compound of the disclosure. A prodrug can be formed when the compound of the disclosure contains a suitable group or substituent to which a property-modifying group can be attached. Examples of prodrugs include derivatives containing in vivo cleavable alkyl or acyl substituents at the sulphonylurea group in a compound of the any one of the Formulae disclosed herein.

Accordingly, the present disclosure includes those compounds of any one of the Formulae disclosed herein as defined hereinbefore when made available by organic synthesis and when made available within the human or animal body by way of cleavage of a prodrug thereof. Accordingly, the present disclosure includes those compounds of any one of the Formulae disclosed herein that are produced by organic synthetic means and also such compounds that are produced in the human or animal body by way of metabolism of a precursor compound, that is a compound of any one of the Formulae disclosed herein may be a synthetically-produced compound or a metabolically-produced compound.

A suitable pharmaceutically acceptable prodrug of a compound of any one of the Formulae disclosed herein is one that is based on reasonable medical judgment as being suitable for administration to the human or animal body without undesirable pharmacological activities and without undue toxicity. Various forms of prodrug have been described, for example in the following documents: a) Methods in Enzymology, Vol. 42, p. 309-396, edited by K. Widder, et al. (Academic Press, 1985); b) Design of Pro-drugs, edited by H. Bundgaard, (Elsevier, 1985); c) A Textbook of Drug Design and Development, edited by Krogsgaard-Larsen and H. Bundgaard, Chapter 5 “Design and Application of Pro-drugs”, by H. Bundgaard p. 113-191 (1991); d) H. Bundgaard, Advanced Drug Delivery Reviews, 8, 1-38 (1992); e) H. Bundgaard, et al., Journal of Pharmaceutical Sciences, 77, 285 (1988); f) N. Kakeya, et al., Chem. Pharm. Bull., 32, 692 (1984); g) T. Higuchi and V. Stella, “Pro-Drugs as Novel Delivery Systems”, A.C.S. Symposium Series, Volume 14; and H) E. Roche (editor), “Bioreversible Carriers in Drug Design”, Pergamon Press, 1987.

A suitable pharmaceutically acceptable prodrug of a compound of any one of the Formulae disclosed herein that possesses a hydroxy group is, for example, an in vivo cleavable ester or ether thereof. An in vivo cleavable ester or ether of a compound of any one of the Formulae disclosed herein containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C₁-C₁₀ alkanoyl groups such as acetyl, benzoyl, phenylacetyl and substituted benzoyl and phenylacetyl groups, C₁-C₁₀ alkoxycarbonyl groups such as ethoxycarbonyl, N,N—(C₁-C₆ alkyl)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁-C₄ alkyl)piperazin-1-ylmethyl. Suitable pharmaceutically acceptable ether forming groups for a hydroxy group include α-acyloxyalkyl groups such as acetoxymethyl and pivaloyloxymethyl groups.

A suitable pharmaceutically acceptable prodrug of a compound of any one of the Formulae disclosed herein that possesses a carboxy group is, for example, an in vivo cleavable amide thereof, for example an amide formed with an amine such as ammonia, a C₁₋₄alkylamine such as methylamine, a (C₁-C₄ alkyl)₂amine such as dimethylamine, N-ethyl-N-methylamine or diethylamine, a C₁-C₄ alkoxy-C₂-C₄ alkylamine such as 2-methoxyethylamine, a phenyl-C₁-C₄ alkylamine such as benzylamine and amino acids such as glycine or an ester thereof.

A suitable pharmaceutically acceptable prodrug of a compound of any one of the Formulae disclosed herein that possesses an amino group is, for example, an in vivo cleavable amide derivative thereof. Suitable pharmaceutically acceptable amides from an amino group include, for example an amide formed with C₁-C₁₀ alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl,piperazin-1-ylmethyl and 4-(C₁-C₄ alkyl)piperazin-1-ylmethyl.

The in vivo effects of a compound of any one of the Formulae disclosed herein may be exerted in part by one or more metabolites that are formed within the human or animal body after administration of a compound of any one of the Formulae disclosed herein. As stated hereinbefore, the in vivo effects of a compound of any one of the Formulae disclosed herein may also be exerted by way of metabolism of a precursor compound (a prodrug).

Suitably, the present disclosure excludes any individual compounds not possessing the biological activity defined herein.

### Methods of Synthesis

In some aspects, the present disclosure provides a method of preparing a compound of the present disclosure.

In some aspects, the present disclosure provides a method of a compound, comprising one or more steps as described herein.

In some aspects, the present disclosure provides a compound obtainable by, or obtained by, or directly obtained by a method for preparing a compound as described herein.

In some aspects, the present disclosure provides an intermediate as described herein, being suitable for use in a method for preparing a compound as described herein.

The compounds of the present disclosure can be prepared by any suitable technique known in the art. Particular processes for the preparation of these compounds are described further in the accompanying examples.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

It will be appreciated that during the synthesis of the compounds of the disclosure in the processes defined herein, or during the synthesis of certain starting materials, it may be desirable to protect certain substituent groups to prevent their undesired reaction. The skilled chemist will appreciate when such protection is required, and how such protecting groups may be put in place, and later removed. For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule. Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium on carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively an arylmethyl group such as a benzyl group may be removed, for example, by hydrogenation over a catalyst such as palladium on carbon.

A suitable protecting group for a carboxy group is, for example, an esterifying group, for example a methyl or an ethyl group which may be removed, for example, by hydrolysis with a base such as sodium hydroxide, or for example a tert-butyl group which may be removed, for example, by treatment with an acid, for example an organic acid such as trifluoroacetic acid, or for example a benzyl group which may be removed, for example, by hydrogenation over a catalyst such as palladium on carbon.

Once a compound of Formula (III) has been synthesised by any one of the processes defined herein, the processes may then further comprise the additional steps of: (i) removing any protecting groups present; (ii) converting the compound Formula (III) into another compound of Formula (III); (iii) forming a pharmaceutically acceptable salt, hydrate or solvate thereof, and/or (iv) forming a prodrug thereof.

The resultant compounds of Formula (III) can be isolated and purified using techniques well known in the art.

Conveniently, the reaction of the compounds is carried out in the presence of a suitable solvent, which is preferably inert under the respective reaction conditions. Examples of suitable solvents comprise but are not limited to hydrocarbons, such as hexane, petroleum ether, benzene, toluene or xylene; chlorinated hydrocarbons, such as trichlorethylene, 1,2-dichloroethane, tetrachloromethane, chloroform or dichloromethane; alcohols, such as methanol, ethanol, isopropanol, n-propanol, n-butanol or tert-butanol; ethers, such as diethyl ether, diisopropyl ether, tetrahydrofuran (THF), 2-methyltetrahydrofuran, cyclopentylmethyl ether (CPME), methyl tert-butyl ether (MTBE) or dioxane; glycol ethers, such as ethylene glycol monomethyl or monoethyl ether or ethylene glycol dimethyl ether (diglyme); ketones, such as acetone, methylisobutylketone (MIBK) or butanone; amides, such as acetamide, dimethylacetamide, dimethylformamide (DMF) or N-methylpyrrolidinone (NMP); nitriles, such as acetonitrile; sulphoxides, such as dimethyl sulphoxide (DMSO); nitro compounds, such as nitromethane or nitrobenzene; esters, such as ethyl acetate or methyl acetate, or mixtures of the said solvents or mixtures with water.

The reaction temperature is suitably between about −100° C. and 300° C., depending on the reaction step and the conditions used.

Reaction times are generally in the range between a fraction of a minute and several days, depending on the reactivity of the respective compounds and the respective reaction conditions. Suitable reaction times are readily determinable by methods known in the art, for example reaction monitoring. Based on the reaction temperatures given above, suitable reaction times generally lie in the range between 10 minutes and 48 hours.

Moreover, by utilising the procedures described herein, in conjunction with ordinary skills in the art, additional compounds of the present disclosure can be readily prepared. Those skilled in the art will readily understand that known variations of the conditions and processes of the following preparative procedures can be used to prepare these compounds.

As will be understood by the person skilled in the art of organic synthesis, compounds of the present disclosure are readily accessible by various synthetic routes, some of which are exemplified in the accompanying examples. The skilled person will easily recognise which kind of reagents and reactions conditions are to be used and how they are to be applied and adapted in any particular instance—wherever necessary or useful—in order to obtain the compounds of the present disclosure. Furthermore, some of the compounds of the present disclosure can readily be synthesised by reacting other compounds of the present disclosure under suitable conditions, for instance, by converting one particular functional group being present in a compound of the present disclosure, or a suitable precursor molecule thereof, into another one by applying standard synthetic methods, like reduction, oxidation, addition or substitution reactions; those methods are well known to the skilled person. Likewise, the skilled person will apply—whenever necessary or useful—synthetic protecting (or protective) groups; suitable protecting groups as well as methods for introducing and removing them are well-known to the person skilled in the art of chemical synthesis and are described, in more detail, in, e.g., P.G.M. Wuts, T.W. Greene, “Greene's Protective Groups in Organic Synthesis”, 4th edition (2006) (John Wiley & Sons).

General routes for the preparation of compounds of Formulae (I)—(III) are described in Schemes 1-2.

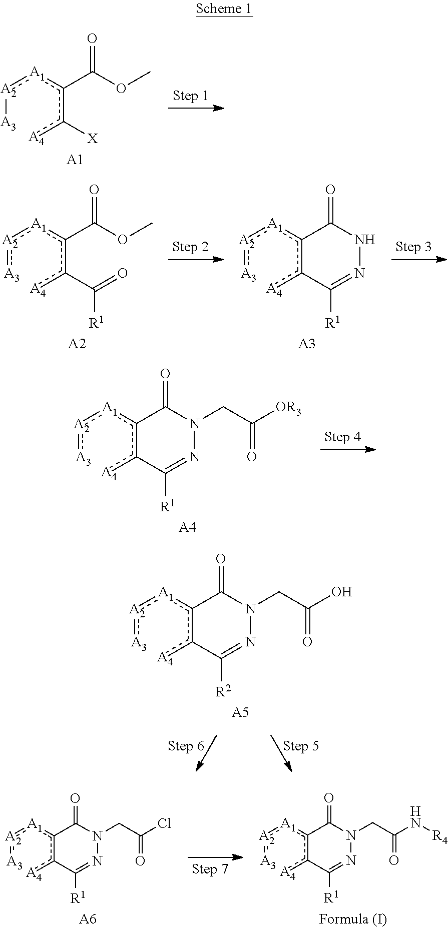

Scheme 1 shows coupling of an appropriately substituted (2-halophenyl)carboxylic ester A1 with an aldehyde under palladium catalysis to form the dicarbonyl species A2. Step 2 is a cyclisation of the dicarbonyl A2 to form the substituted phthalazin-1(2H)-one A3. Alkylation is achieved with a bromo or chloro acetate ester to yield A4. Hydrolysis provides the acid A5, which may be either treated directly with an appropriate amine under amide coupling conditions, using a coupling reagent such as hexafluorophosphate azabenzotriazole tetramethyl uronium (HATU) and an appropriate base, or alternatively transformed to an acid chloride using a chlorinating agent such as oxalyl chloride, thionyl chloride or POCl₃, followed by treatment with an amine to form compound Formula (III).

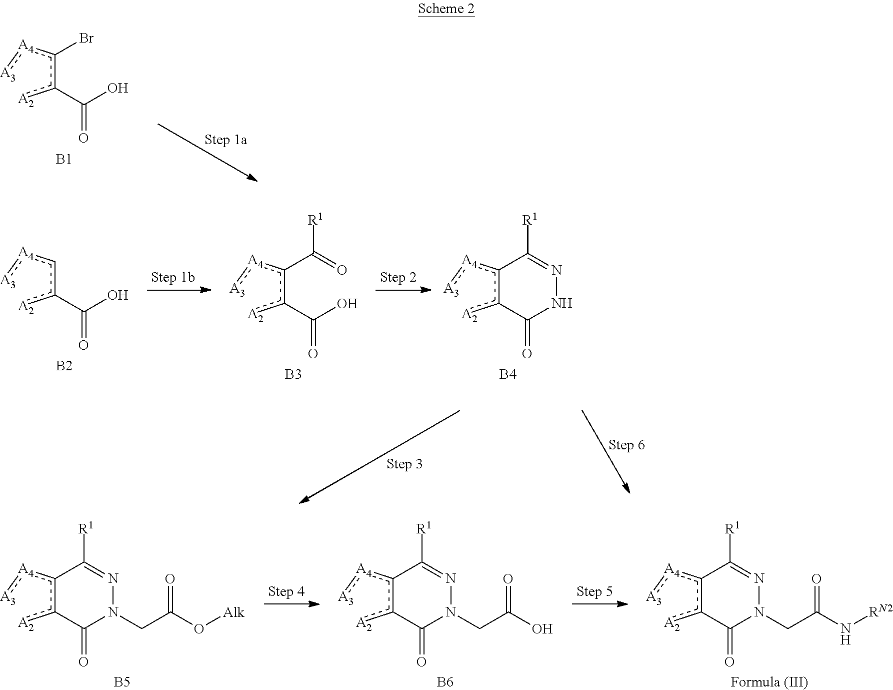

Compounds of Formula (III) can be prepared according to Scheme 2. Bromo heteroaryl B1 can be treated with n-butyllithium followed by a Weinreb amide to give B3. Alternatively, B3 can be synthesised from B2 by treating with an organometallic base (e.g., LDA) followed by a Weinreb amide. B3 can be treated with hydrazine hydrate in an appropriate solvent (e.g., ethanol) at an elevated temperature to give B4. Compounds of Formula (III) can be synthesised by treating B4 with the appropriate alkylating agent in the presence of a base. B4 can also be alkylated, for example with ethylbromoacetate, to give B5 (where Alk represents an alkyl group). B5 can be hydrolysed to give B6. Compounds of Formula (III) can be synthesised from B6 using amide coupling conditions.

### Biological Assays

Compounds designed, selected and/or optimised by methods described above, once produced, can be characterised using a variety of assays known to those skilled in the art to determine whether the compounds have biological activity. For example, the molecules can be characterised by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

Various in vitro or in vivo biological assays may be suitable for detecting the effect of the compounds of the present disclosure. These in vitro or in vivo biological assays can include, but are not limited to, enzymatic activity assays, electrophoretic mobility shift assays, reporter gene assays, in vitro cell viability assays, and the assays described herein.

In some embodiments, the biological away is a biological away testing inhibitory activity against IL-1β release upon NLRP3 activation in peripheral blood mononuclear cells (PBMC).

In some embodiments, the biological assay is a PBMC IC₅₀ Determination Assay. In some embodiments, the biological assay is a PBMC IC₅₀ Determination Assay described in Example 36.

In some embodiments, the compounds of the present disclosure may be tested for their inhibitory activity against IL-1β release upon NLRP3 activation in blood cells (e.g., peripheral blood mononuclear cells (PBMC)).

In some embodiments, PBMC may be isolated and seeded into the wells of a plate and incubated for a period of time (e.g., for 3 hours with a lipopolysaccharide). Following incubation, the medium may be exchanged and a compound added to the well (e.g., a compound of the present disclosure) and the cells may be incubated. Next, the cells may be stimulated (e.g., with ATP or nigericin) and the cell culture media collected for further analysis.

In some embodiments, the release of IL-1β into the media may be determined by a quantitative detection of IL-1β in the media (e.g., using ELISA).

In some embodiments, PBMC may be isolated (e.g., from buffy coats). Isolated cells may be seeded into wells and incubated (e.g., for 3 hours with lipopolysaccharide). The compounds of the present disclosure may then be added and the cells incubated. Next, the cells may be stimulated and the media from the wells collected for further analysis.

In some embodiments, the release of IL-1β into the media may be determined by quantitative detection (e.g., of IL-1β in media using HTRF®).

### Pharmaceutical Compositions

In some aspects, the present disclosure provides a pharmaceutical composition comprising a compound of the present disclosure as an active ingredient.

In some embodiments, the present disclosure provides a pharmaceutical composition comprising a compound described herein and one or more pharmaceutically acceptable carriers or excipients. In some embodiments, the present disclosure provides a pharmaceutical composition comprising at least one compound selected from Table 1.

As used herein, the term “composition” is intended to encompass a product comprising the specified ingredients in the specified amounts, as well as any product which results, directly or indirectly, from combination of the specified ingredients in the specified amounts.

The compounds of present disclosure can be formulated for oral administration in forms such as tablets, capsules (each of which includes sustained release or timed-release formulations), pills, powders, granules, elixirs, tinctures, suspensions, syrups and emulsions. The compounds of present disclosure on can also be formulated for intravenous (bolus or in-fusion), intraperitoneal, topical, subcutaneous, intra-muscular or transdermal (e.g., patch) administration, all using forms well known to those of ordinary skill in the pharmaceutical arts.

The formulation of the present disclosure may be in the form of an aqueous solution comprising an aqueous vehicle. The aqueous vehicle component may comprise water and at least one pharmaceutically acceptable excipient. Suitable acceptable excipients include those selected from the group consisting of a solubility enhancing agent, chelating agent, preservative, tonicity agent, viscosity/suspending agent, buffer, and pH modifying agent, and a mixture thereof.

Any suitable solubility enhancing agent can be used. Examples of a solubility enhancing agent include cyclodextrin, such as those selected from the group consisting of hydroxypropyl-β-cyclodextrin, methyl-β-cyclodextrin, randomly methylated-β-cyclodextrin, ethylated-β-cyclodextrin, triacetyl-β-cyclodextrin, peracetylated-β-cyclodextrin, carboxymethyl-β-cyclodextrin, hydroxyethyl-β-cyclodextrin, 2-hydroxy-3-(trimethylammonio)propyl-β-cyclodextrin, glucosyl-β-cyclodextrin, sulphated β-cyclodextrin (S-β-CD), maltosyl-β-cyclodextrin, β-cyclodextrin sulphobutyl ether, branched-β-cyclodextrin, hydroxypropyl-γ-cyclodextrin, randomly methylated-γ-cyclodextrin, and trimethyl-γ-cyclodextrin, and mixtures thereof.

Any suitable chelating agent can be used. Examples of a suitable chelating agent include those selected from the group consisting of ethylenediaminetetraacetic acid and metal salts thereof, disodium edetate, trisodium edetate, and tetrasodium edetate, and mixtures thereof.

Any suitable preservative can be used. Examples of a preservative include those selected from the group consisting of quaternary ammonium salts such as benzalkonium halides (preferably benzalkonium chloride), chlorhexidine gluconate, benzethonium chloride, cetyl pyridinium chloride, benzyl bromide, phenylmercury nitrate, phenylmercury acetate, phenylmercury neodecanoate, merthiolate, methylparaben, propylparaben, sorbic acid, potassium sorbate, sodium benzoate, sodium propionate, ethyl p-hydroxybenzoate, propylaminopropyl biguanide, and butyl-p-hydroxybenzoate, and sorbic acid, and mixtures thereof.

The aqueous vehicle may also include a tonicity agent to adjust the tonicity (osmotic pressure). The tonicity agent can be selected from the group consisting of a glycol (such as propylene glycol, diethylene glycol, triethylene glycol), glycerol, dextrose, glycerin, mannitol, potassium chloride, and sodium chloride, and a mixture thereof.

The aqueous vehicle may also contain a viscosity/suspending agent. Suitable viscosity/suspending agents include those selected from the group consisting of cellulose derivatives, such as methyl cellulose, ethyl cellulose, hydroxyethylcellulose, polyethylene glycols (such as polyethylene glycol 300, polyethylene glycol 400), carboxymethyl cellulose, hydroxypropylmethyl cellulose, and cross-linked acrylic acid polymers (carbomers), such as polymers of acrylic acid cross-linked with polyalkenyl ethers or divinyl glycol (Carbopols—such as Carbopol 934, Carbopol 934P, Carbopol 971, Carbopol 974 and Carbopol 974P), and a mixture thereof.

In order to adjust the formulation to an acceptable pH (typically a pH range of about 5.0 to about 9.0, more preferably about 5.5 to about 8.5, particularly about 6.0 to about 8.5, about 7.0 to about 8.5, about 7.2 to about 7.7, about 7.1 to about 7.9, or about 7.5 to about 8.0), the formulation may contain a pH modifying agent. The pH modifying agent is typically a mineral acid or metal hydroxide base, selected from the group of potassium hydroxide, sodium hydroxide, and hydrochloric acid, and mixtures thereof, and preferably sodium hydroxide and/or hydrochloric acid. These acidic and/or basic pH modifying agents are added to adjust the formulation to the target acceptable pH range. Hence it may not be necessary to use both acid and base—depending on the formulation, the addition of one of the acid or base may be sufficient to bring the mixture to the desired pH range.

The aqueous vehicle may also contain a buffering agent to stabilise the pH. When used, the buffer is selected from the group consisting of a phosphate buffer (such as sodium dihydrogen phosphate and disodium hydrogen phosphate), a borate buffer (such as boric acid, or salts thereof including disodium tetraborate), a citrate buffer (such as citric acid, or salts thereof including sodium citrate), and F-aminocaproic acid, and mixtures thereof.

The formulation may further comprise a wetting agent. Suitable classes of wetting agents include those selected from the group consisting of polyoxypropylene-polyoxyethylene block copolymers (poloxamers), polyethoxylated ethers of castor oils, polyoxyethylenated sorbitan esters (polysorbates), polymers of oxyethylated octyl phenol (Tyloxapol), polyoxyl 40 stearate, fatty acid glycol esters, fatty acid glyceryl esters, sucrose fatty esters, and polyoxyethylene fatty esters, and mixtures thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavouring agent such as peppermint, methyl salicylate, or orange flavoring.

According to a further aspect of the disclosure there is provided a pharmaceutical composition which comprises a compound of the disclosure as defined hereinbefore, or a pharmaceutically acceptable salt, hydrate or solvate thereof, in association with a pharmaceutically acceptable diluent or carrier.

The compositions of the disclosure may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the disclosure may be obtained by conventional procedures using conventional pharmaceutical excipients, well known in the art. Thus, compositions intended for oral use may contain, for example, one or more colouring, sweetening, flavouring and/or preservative agents.

An effective amount of a compound of the present disclosure for use in therapy is an amount sufficient to treat or prevent an inflammasome related condition referred to herein, slow its progression and/or reduce the symptoms associated with the condition.

The size of the dose for therapeutic or prophylactic purposes of a compound of Formula (III) will naturally vary according to the nature and severity of the conditions, the age and sex of the animal or patient and the route of administration, according to well-known principles of medicine.

### Methods of Use

In some aspects, the present disclosure provides a method of inhibiting inflammasome (e.g., the NLRP3 inflammasome) activity (e.g., in vitro or in vivo), comprising contacting a cell with an effective amount of a compound of the present disclosure or a pharmaceutically acceptable salt thereof.

In some aspects, the present disclosure provides a method of treating or preventing a disease or disorder disclosed herein in a subject in need thereof, comprising administering to the subject a compound of the present disclosure or a pharmaceutically acceptable salt thereof, or a pharmaceutical composition of the present disclosure.

In some aspects, the present disclosure provides a method of treating or preventing a disease or disorder disclosed herein in a subject in need thereof, comprising administering to the subject a therapeutically effective amount of a compound of the present disclosure or a pharmaceutically acceptable salt thereof, or a pharmaceutical composition of the present disclosure.

In some embodiments, the disease or disorder is associated with an implicated inflammasome activity. In some embodiments, the disease or disorder is a disease or disorder in which inflammasome activity is implicated.

In some embodiments, the disease or disorder is an inflammatory disorder, autoinflammatory disorder, an autoimmune disorder, a neurodegenerative disease, or cancer.

In some embodiments, the disease or disorder is an inflammatory disorder, autoinflammatory disorder and/or an autoimmune disorder.

In some embodiments, the disease or disorder is cytokine release syndrome (CRS).

In some embodiments, the disease or disorder is selected from cryopyrin-associated autoinflammatory syndrome (CAPS; e.g., familial cold autoinflammatory syndrome (FCAS), Muckle-Wells syndrome (MWS), chronic infantile neurological cutaneous and articular (CINCA) syndrome/neonatal-onset multisystem inflammatory disease (NOMID)), familial Mediterranean fever (FMF), nonalcoholic fatty liver disease (NAFLD), non-alcoholic steatohepatitis (NASH), gout, rheumatoid arthritis, osteoarthritis, Crohn's disease, chronic obstructive pulmonary disease (COPD), chronic kidney disease (CKD), fibrosis, obesity, type 2 diabetes, multiple sclerosis, dermatological disease (e.g. acne) and neuroinflammation occurring in protein misfolding diseases (e.g., Prion diseases).

In some embodiments, the disease or disorder is a neurodegenerative disease.

In some embodiments, the disease or disorder is Parkinson's disease or Alzheimer's disease.

In some embodiments, the disease or disorder is a dermatological disease.

In some embodiments, the dermatological disease is acne.

In some embodiments, the disease or disorder is cancer.

In some embodiments, the cancer is metastasising cancer, gastrointestinal cancer, skin cancer, non-small-cell lung carcinoma, brain cancer (e.g., glioblastoma) or colorectal adenocarcinoma.

In some aspects, the present disclosure provides a method of treating or preventing an autoinflammatory disorder, an autoimmune disorder, a neurodegenerative disease or cancer in a subject in need thereof, comprising administering to the subject a compound of the present disclosure or a pharmaceutically acceptable salt thereof, or a pharmaceutical composition of the present disclosure.

In some aspects, the present disclosure provides a method of treating or preventing an autoinflammatory disorder, an autoimmune disorder, a neurodegenerative disease or cancer in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing an inflammatory disorder, autoinflammatory disorder and/or an autoimmune disorder selected from cryopyrin-associated autoinflammatory syndrome (CAPS; e.g., familial cold autoinflammatory syndrome (FCAS), Muckle-Wells syndrome (MWS), chronic infantile neurological cutaneous and articular (CINCA) syndrome/neonatal-onset multisystem inflammatory disease (NOMID)), familial Mediterranean fever (FMF), nonalcoholic fatty liver disease (NAFLD), non-alcoholic steatohepatitis (NASH), gout, rheumatoid arthritis, osteoarthritis, Crohn's disease, chronic obstructive pulmonary disease (COPD), chronic kidney disease (CKD), fibrosis, obesity, type 2 diabetes, multiple sclerosis, dermatological disease (e.g. acne) and neuroinflammation occurring in protein misfolding diseases (e.g., Prion diseases) in a subject in need thereof, comprising administering to the subject a compound of the present disclosure or a pharmaceutically acceptable salt thereof, or a pharmaceutical composition of the present disclosure.

In some aspects, the present disclosure provides a method of treating or preventing an inflammatory disorder, autoinflammatory disorder and/or an autoimmune disorder selected from cryopyrin-associated autoinflammatory syndrome (CAPS; e.g., familial cold autoinflammatory syndrome (FCAS), Muckle-Wells syndrome (MWS), chronic infantile neurological cutaneous and articular (CINCA) syndrome/neonatal-onset multisystem inflammatory disease (NOMID)), familial Mediterranean fever (FMF), nonalcoholic fatty liver disease (NAFLD), non-alcoholic steatohepatitis (NASH), gout, rheumatoid arthritis, osteoarthritis, Crohn's disease, chronic obstructive pulmonary disease (COPD), chronic kidney disease (CKD), fibrosis, obesity, type 2 diabetes, multiple sclerosis, dermatological disease (e.g. acne) and neuroinflammation occurring in protein misfolding diseases (e.g., Prion diseases) in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing cytokine release syndrome (CRS) in a subject in need thereof, comprising administering to the subject a compound of the present disclosure or a pharmaceutically acceptable salt thereof, or a pharmaceutical composition of the present disclosure. In some embodiments, the CRS is associated with COVID-19. In some embodiments, the CRS is associated with adoptive cell therapy.

In some aspects, the present disclosure provides a method of treating or preventing cytokine release syndrome (CRS) in a subject in need thereof, comprising administering to the subject a therapeutically effective amount of a compound of the present disclosure or a pharmaceutically acceptable salt thereof, or a pharmaceutical composition of the present disclosure. In some embodiments, the CRS is associated with COVID-19. In some embodiments, the CRS is associated with adoptive cell therapy.

In some aspects, the present disclosure provides a method of treating or preventing a neurodegenerative disease (e.g., Parkinson's disease or Alzheimer's disease) in a subject in need thereof, said method comprising administering to the subject a compound of the present disclosure or a pharmaceutically acceptable salt thereof, or a pharmaceutical composition of the present disclosure.

In some aspects, the present disclosure provides a method of treating or preventing a neurodegenerative disease (e.g., Parkinson's disease or Alzheimer's disease) in a subject in need thereof, said method comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing cancer in a subject in need thereof, said method comprising administering to the subject a compound of the present disclosure or a pharmaceutically acceptable salt thereof, or a pharmaceutical composition of the present disclosure.

In some aspects, the present disclosure provides a method of treating or preventing cancer in a subject in need thereof, said method comprising administering to the subject a therapeutically effective amount of a compound of the present disclosure or a pharmaceutically acceptable salt thereof, or a pharmaceutical composition of the present disclosure.

In some aspects, the present disclosure provides a compound of the present disclosure or a pharmaceutically acceptable salt thereof for use in inhibiting inflammasome (e.g., the NLRP3 inflammasome) activity (e.g., in vitro or in vivo).

In some aspects, the present disclosure provides a compound of the present disclosure or a pharmaceutically acceptable salt thereof for use in treating or preventing a disease or disorder disclosed herein.

In some aspects, the present disclosure provides a compound of the present disclosure or a pharmaceutically acceptable salt thereof for use in treating or preventing an inflammatory disorder, an autoinflammatory disorder, an autoimmune disorder, a neurodegenerative disease or cancer in a subject in need thereof.

In some aspects, the present disclosure provides a compound of the present disclosure or a pharmaceutically acceptable salt thereof for use in treating or preventing an inflammatory disorder, an autoinflammatory disorder and/or an autoimmune disorder selected from cryopyrin-associated autoinflammatory syndrome (CAPS; e.g., familial cold autoinflammatory syndrome (FCAS), Muckle-Wells syndrome (MWS), chronic infantile neurological cutaneous and articular (CINCA) syndrome/neonatal-onset multisystem inflammatory disease (NOMID)), familial Mediterranean fever (FMF), nonalcoholic fatty liver disease (NAFLD), non-alcoholic steatohepatitis (NASH), gout, rheumatoid arthritis, osteoarthritis, Crohn's disease, chronic obstructive pulmonary disease (COPD), chronic kidney disease (CKD), fibrosis, obesity, type 2 diabetes, multiple sclerosis and neuroinflammation occurring in protein misfolding diseases (e.g., Prion diseases) in a subject in need thereof.

In some aspects, the present disclosure provides a compound of the present disclosure or a pharmaceutically acceptable salt thereof for use in treating or preventing CRS in a subject in need thereof.

In some aspects, the present disclosure provides a compound of the present disclosure or a pharmaceutically acceptable salt thereof for use in treating or preventing a neurodegenerative disease (e.g., Parkinson's disease or Alzheimer's disease) in a subject in need thereof.

In some aspects, the present disclosure provides a compound of the present disclosure or a pharmaceutically acceptable salt thereof for use in treating or preventing cancer in a subject in need thereof.

In some aspects, the present disclosure provides use of a compound of the present disclosure or a pharmaceutically acceptable salt thereof in the manufacture of a medicament for inhibiting inflammasome (e.g., the NLRP3 inflammasome) activity (e.g., in vitro or in vivo).

In some aspects, the present disclosure provides use of a compound of the present disclosure or a pharmaceutically acceptable salt thereof in the manufacture of a medicament for treating or preventing a disease or disorder disclosed herein.

In some aspects, the present disclosure provides use of a compound of the present disclosure or a pharmaceutically acceptable salt thereof in the manufacture of a medicament for treating or preventing an inflammatory disorder, an autoinflammatory disorder, an autoimmune disorder, a neurodegenerative disease or cancer in a subject in need thereof.

In some aspects, the present disclosure provides use of a compound of the present disclosure or a pharmaceutically acceptable salt thereof in the manufacture of a medicament for treating or preventing an inflammatory disorder, an autoinflammatory disorder and/or an autoimmune disorder selected from cryopyrin-associated autoinflammatory syndrome (CAPS; e.g., familial cold autoinflammatory syndrome (FCAS), Muckle-Wells syndrome (MWS), chronic infantile neurological cutaneous and articular (CINCA) syndrome/neonatal-onset multisystem inflammatory disease (NOMID)), familial Mediterranean fever (FMF), nonalcoholic fatty liver disease (NAFLD), non-alcoholic steatohepatitis (NASH), gout, rheumatoid arthritis, osteoarthritis, Crohn's disease, chronic obstructive pulmonary disease (COPD), chronic kidney disease (CKD), fibrosis, obesity, type 2 diabetes, multiple sclerosis, dermatological disorders (e.g., acne) and neuroinflammation occurring in protein misfolding diseases (e.g., Prion diseases) in a subject in need thereof.

In some aspects, the present disclosure provides use of a compound of the present disclosure or a pharmaceutically acceptable salt thereof in the manufacture of a medicament for treating or preventing CRS in a subject in need thereof.

In some aspects, the present disclosure provides use of a compound of the present disclosure or a pharmaceutically acceptable salt thereof in the manufacture of a medicament for treating or preventing a neurodegenerative disease (e.g., Parkinson's disease or Alzheimer's disease) in a subject in need thereof.

In some aspects, the present disclosure provides use of a compound of the present disclosure or a pharmaceutically acceptable salt thereof in the manufacture of a medicament for treating or preventing cancer in a subject in need thereof.

The present disclosure provides compounds that function as inhibitors of inflammasome activity. The present disclosure therefore provides a method of inhibiting inflammasome activity in vitro or in vivo, said method comprising contacting a cell with an effective amount of a compound, or a pharmaceutically acceptable salt thereof, as defined herein.

Effectiveness of compounds of the disclosure can be determined by industry-accepted assays/disease models according to standard practices of elucidating the same as described in the art and are found in the current general knowledge.

The present disclosure also provides a method of treating a disease or disorder in which inflammasome activity is implicated in a patient in need of such treatment, said method comprising administering to said patient a compound, or a pharmaceutically acceptable salt thereof, or a pharmaceutical composition as defined herein.

The present disclosure also provides a method of treating a disease or disorder in which inflammasome activity is implicated in a patient in need of such treatment, said method comprising administering to said patient a therapeutically effective amount of a compound, or a pharmaceutically acceptable salt thereof, or a pharmaceutical composition as defined herein.

On a general level, the compounds of the present disclosure, which inhibit the maturation of cytokines of the IL-1 family, are effective in all therapeutic indications that are mediated or associated with elevated levels of active forms of cytokines belonging to IL-1 family of cytokines (Sims J. et al. Nature Reviews Immunology 10, 89-102 (February 2010).

Exemplary diseases and the corresponding references will be given in the following: inflammatory, autoinflammatory and autoimmune diseases like CAPS (Dinarello, C. A. Immunity. 2004 March; 20(3):243-4; Hoffman, H. M. et al. Reumatologia 2005; 21(3)), gout, rheumatoid arthritis (Gabay, C. et al. Arthritis Research & Therapy 2009, 11:230; Schett, G. et al. Nat Rev Rheumatol. 2016 January; 12(1):14-24.), Crohn's disease (Jung Mogg Kim Korean J. Gastroenterol. Vol. 58 No. 6, 300-310), COPD (Mortaz, E. et al. Tanaffos. 2011; 10(2): 9-14.), fibrosis (Gasse, P. et al. Am. J. Respir. Crit. Care Med. 2009 May 15; 179(10):903-13), obesity, type 2 diabetes ((Dinarello, C. A. et al. Curr. Opin. Endocrinol. Diabetes Obes. 2010 August; 17(4):314-21)) multiple sclerosis (see EAE-model in Coll, R. C. et al. Nat. Med. 2015 March; 21(3):248-55) and many others (Martinon, F. et al. Immunol. 2009. 27:229-65) like Parkinson's disease or Alzheimer's disease (Michael, T. et al. Nature 493, 674-678 (31 Jan. 2013); Halle, A. et al., Nat. Immunol. 2008 August; 9(8):857-65; Saresella, M. et al. Mol. Neurodegener. 2016 Mar. 3; 11:23) and some oncological disorders.

Suitably, the compounds according to the present disclosure can be used for the treatment of a disease selected from the group consisting of cytokine release syndrome (CRS), an inflammatory disease, an autoinflammatory disease, an autoimmune disease, a neurodegenerative disease and cancer. Said inflammatory, autoinflammatory and autoimmune disease is suitably selected from the group consisting of a cryopyrin-associated autoinflammatory syndrome (CAPS, such as for example familial cold autoinflammatory syndrome (FCAS), Muckle-Wells syndrome (MWS), chronic infantile neurological cutaneous and articular (CINCA) syndrome/neonatal-onset multisystem inflammatory disease (NOMID)), familial Mediterranean fever (FMF), nonalcoholic fatty liver disease (NAFLD), non-alcoholic steatohepatitis (NASH), chronic kidney disease (CKD), gout, rheumatoid arthritis, osteoarthritis, Crohn's disease, COPD, fibrosis, obesity, type 2 diabetes, multiple sclerosis, dermatological diseases (e.g., acne) and neuroinflammation occurring in protein misfolding diseases, such as Prion diseases. Said neurodegenerative disease includes, but is not limited, to Parkinson's disease and Alzheimer's disease.

Accordingly, the compounds of the present disclosure can be used for the treatment of a disease selected from the group consisting of cryopyrin-associated autoinflammatory syndrome (CAPS, such as for example familial cold autoinflammatory syndrome (FCAS), Muckle-Wells syndrome (MWS), chronic infantile neurological cutaneous and articular (CINCA) syndrome/neonatal-onset multisystem inflammatory disease (NOMID)), familial Mediterranean fever (FMF), nonalcoholic fatty liver disease (NAFLD), non-alcoholic steatohepatitis (NASH), chronic kidney disease (CKD), gout, rheumatoid arthritis, osteoarthritis, Crohn's disease, COPD, fibrosis, obesity, type 2 diabetes, multiple sclerosis, dermatological diseases (e.g., acne) neuroinflammation occurring in protein misfolding diseases, such as Prion diseases, neurogenerative diseases (e.g., Parkinson's disease, Alzheimer's disease) and oncological disorders.

Inflammatory Disease Associated with Infection

In some embodiments, the disease or disorder is an inflammatory disease.

In some embodiments, the inflammatory disease is associated with an infection.

In some embodiments, the inflammatory disease is associated with an infection by a virus.

In some embodiments, the inflammatory disease is associated with an infection by an RNA virus. In some embodiments, the RNA virus is a single stranded RNA virus. Single stranded RNA viruses include group IV (positive strand) and group V (negative strand) single stranded RNA viruses. Group IV viruses include coronaviruses.

In some embodiments, the inflammatory disease is associated with an infection by a coronavirus. In some embodiments, the coronavirus is Severe Acute Respiratory Syndrome Coronavirus 2 (SARS-CoV 2), SARS coronavirus (SARS CoV) or Middle East respiratory syndrome-related coronavirus (MERS).

In some embodiments, the inflammatory disease is associated with an infection by SARS-CoV 2. In some embodiments, SARS-CoV 2 infection leads to 2019 novel coronavirus disease (COVID-19).

In some embodiments, the inflammatory disease is an inflammatory disease of lung.

In some embodiments, the inflammatory disease of lung is associated with an infection by SARS-CoV 2.

In some embodiments, the inflammatory disease comprises cytokine release syndrome (CRS).

In some embodiments, the cytokine release syndrome (CRS) is associated with an infection by SARS-CoV 2.

### Cytokine Release Syndrome and Immunotherapy

In some embodiments, the disease or disorder is an inflammatory disease.

In some embodiments, the inflammatory disease is associated with an immunotherapy.

In some embodiments, the immunotherapy causes cytokine release syndrome (CRS).

The effectiveness of immunotherapies, such as CAR-T, are hampered by the frequency with which such therapies induce cytokine release syndrome. Without wishing to be bound by theory, it is thought that the severity of CRS induced by immunotherapy is mediated by IL-6, IL-1 and NO production (Giavridis et al. Nature Medicine; doi.org/10.1038/s41591-018-0041-7). Alternatively, or in addition, CRS may occur when cells targeted by the adoptive cell therapy undergo pyroptosis, a highly inflammatory form of programmed cell death. Pyroptosis leads to release of factors that stimulate macrophages to produce pro-inflammatory cytokines, leading to CRS (Liu et al. Science Immunology (2020) V: eeax7969).

In some embodiments, the immunotherapy comprises an antibody or an adoptive cell therapy.

In some embodiments, the adoptive cell therapy comprises a CAR-T or TCR-T cell therapy.

In some embodiments, the adoptive cell therapy comprises a cancer therapy. For example, the cancer therapy can be to treat B cell lymphoma or B cell acute lymphoblastic leukemia. For example, the adoptive cells may express a CAR targeting CD19+ B cell acute lymphoblastic leukemia cells.

In some embodiments, the adoptive cell therapy comprises administration of T cells, B cells or NK cells.

In some embodiments, the adoptive cell therapy is autologous.

In some embodiments, the adoptive therapy is allogeneic.

Treatment in Cancer; Links with Inflammasome

Chronic inflammation responses have long been observed to be associated with various types of cancer. During malignant transformation or cancer therapy inflammasomes may become activated in response to danger signals and this activation may be both beneficial and detrimental in cancer.

IL-1β expression is elevated in a variety of cancers (including breast, prostate, colon, lung, head and neck cancers and melanomas) and patients with IL-1β producing tumours generally have a worse prognosis (Lewis, Anne M., et al. “Interleukin-1 and cancer progression: the emerging role of interleukin-1 receptor antagonist as a novel therapeutic agent in cancer treatment.” Journal of translational medicine 4.1 (2006): 48).

Cancers derived from epithelial cells (carcinoma) or epithelium in glands (adenocarcinoma) are heterogeneous; consisting of many different cell types. This may include fibroblasts, immune cells, adipocytes, endothelial cells and pericytes amongst others, all of which may be cytokine/chemokine secreting (Grivennikov, Sergei I., Florian R. Greten, and Michael Karin. “Immunity, inflammation, and cancer.” Cell 140.6 (2010): 883-899). This can lead to cancer-associated inflammation through the immune cell infiltration. The presence of leukocytes in tumours is known but it has only recently become evident that an inflammatory microenvironment is an essential component of all tumours. Most tumours (>90%) are the result of somatic mutations or environmental factors rather than germline mutations and many environmental causes of cancer are associated with chronic inflammation (20% of cancers are related to chronic infection, 30% to smoking/inhaled pollutants and 35% to dietary factors (20% of all cancers are linked to obesity) (Aggarwal, Bharat B., R. V. Vijayalekshmi, and Bokyung Sung. “Targeting inflammatory pathways for prevention and therapy of cancer: short-term friend, long-term foe.” Clinical Cancer Research 15.2 (2009): 425-430).

### GI Cancers

Cancers of the gastrointestinal (GI) tract are frequently associated with chronic inflammation. For example, H. pylori infection is associated with gastric cancer (Amieva, Manuel, and Richard M. Peek. “Pathobiology of Helicobacter pylori-Induced Gastric Cancer.” Gastroenterology 150.1 (2016): 64-78). Colorectal cancer is associated with inflammatory bowel disease (Bernstein, Charles N., et al. “Cancer risk in patients with inflammatory bowel disease.” Cancer 91.4 (2001): 854-862). Chronic inflammation in stomach leads to the upregulation of IL-1 and other cytokines (Basso, D. et al., (1996) Helicobacter pylori infection enhances mucosal interleukin-1 beta, interleukin-6, and the soluble receptor of interleukin-2. Int J Clin Lab Res 26:207-210) and polymorphisms in IL-1β gene can increase risk of gastric cancer (Wang, P. et al., (2007) Association of interleukin-1 gene polymorphisms with gastric cancer: a meta-analysis. Int J Cancer 120:552-562).

In 19% of gastric cancer cases, caspase-1 expression is decreased which correlates with stage, lymph node metastasis and survival (Jee et al., 2005). Mycoplasma hyorhinis is associated with the development of gastric cancer its activation of the NLRP3 inflammasome may be associated with its promotion of gastric cancer metastasis (Xu et al., 2013).

### Skin Cancers

Ultraviolet radiation is the greatest environmental risk for skin cancer which is promoted by causing DNA damage, immunosuppression and inflammation. The most malignant skin cancer, melanoma, is characterised by the upregulation of inflammatory cytokines, all of which can be regulated by IL-1β (Lázár-Molnár, Eszter, et al. “Autocrine and paracrine regulation by cytokines and growth factors in melanoma.” Cytokine 12.6 (2000): 547-554). Systemic inflammation induces an enhancement of melanoma cell metastasis and growth by IL-1-dependent mechanisms in vivo. Using thymoquinone inhibition of metastasis in a B16F10 mouse melanoma model was shown to be dependent on inhibition of the NLRP3 inflammasome (Ahmad, Israr, et al. “Thymoquinone suppresses metastasis of melanoma cells by inhibition of NLRP3 inflammasome.” Toxicology and applied pharmacology 270.1 (2013): 70-76).

### Glioblastoma

NLRP3 contributes to radiotherapy resistance in glioma. Ionising radiation can induce NLRP3 expression whereas NLRP3 inhibition reduced tumour growth and prolonged mouse survival following radiation therapy. NLRP3 inflammasome inhibition can therefore provide a therapeutic strategy for radiation-resistant glioma (Li, Lianling, and Yuguang Liu. “Aging-related gene signature regulated by Nlrp3 predicts glioma progression.” American journal of cancer research 5.1 (2015): 442).

### Metastasis

More widely, NLRP3 is considered by the applicants to be involved in the promotion of metastasis and consequently modulation of NLRP3 should plausibly block this. IL-1 is involved in tumour genesis, tumour invasiveness, metastasis, tumour host interactions (Apte, Ron N., et al. “The involvement of IL-1 in tumorigenesis, tumour invasiveness, metastasis and tumour-host interactions.” Cancer and Metastasis Reviews 25.3 (2006): 387-408) and angiogenesis (Voronov, Elena, et al. “IL-1 is required for tumor invasiveness and angiogenesis.” Proceedings of the National Academy of Sciences 100.5 (2003): 2645-2650).

The IL-1 gene is frequently expressed in metastases from patients with several types of human cancers. For example, IL-1mRNA was highly expressed in more than half of all tested metastatic human tumour specimens including specifically non-small-cell lung carcinoma, colorectal adenocarcinoma, and melanoma tumour samples (Elaraj, Dina M., et al. “The role of interleukin 1 in growth and metastasis of human cancer xenografts.” Clinica Cancer Research 12.4 (2006): 1088-1096) and IL-1RA inhibits xenograft growth in IL-1 producing tumours but without anti-proliferative effects in vitro.

Further, IL-1 signalling is a biomarker for predicting breast cancer patients at increased risk for developing bone metastasis. In mouse models IL-1β and its receptor are upregulated in breast cancer cells that metastasise to bone compared with cells that do not. In a mouse model the IL-1 receptor antagonist anakinra reduced proliferation and angiogenesis in addition to exerting significant effects on the tumour environment reducing bone turnover markers, IL-1β and TNF alpha (Holen, Ingunn, et al. “IL-1 drives breast cancer growth and bone metastasis in vivo.” Oncotarget (2016).

IL-18 induced the production of MMP-9 in the human leukaemia cell line HL-60, thus favouring degradation of the extracellular matrix and the migration and invasiveness of cancer cells (Zhang, Bin, et al. “IL-18 increases invasiveness of HL-60 myeloid leukemia cells: up-regulation of matrix metalloproteinases-9 (MMP-9) expression.” Leukemia research 28.1 (2004): 91-95). Additionally IL-18 can support the development of tumour metastasis in the liver by inducing expression of VCAM-1 on hepatic sinusoidal endothelium (Carrascal, Maria Teresa, et al. “Interleukin-18 binding protein reduces b16 melanoma hepatic metastasis by neutralizing adhesiveness and growth factors of sinusoidal endothelium.” Cancer Research 63.2 (2003): 491-497).

### CD36

The fatty acid scavenger receptor CD36 serves a dual role in priming gene transcription of pro-IL-1β and inducing assembly of the NLRP3 inflammasome complex. CD36 and the TLR4-TLR6 heterodimer recognise oxLDL, which initiates a signalling pathway leading to transcriptional upregulation of NLRP3 and pro-IL-1β (signal 1). CD36 also mediates the internalisation of oxLDL into the lysosomal compartment, where crystals are formed that induce lysosomal rupture and activation of the NLRP3 inflammasome (signal 2) (Kagan, J. and Horng T., “NLRP3 inflammasome activation: CD36 serves double duty.” Nature immunology 14.8 (2013): 772-774).

A subpopulation of human oral carcinoma cells express high levels of the fatty acid scavenger receptor CD36 and are unique in their ability to initiate metastasis. Palmitic acid or a high fat diet boosted the metastatic potential of the CD36+ cells. Neutralising anti-CD36 antibodies blocked metastasis in orthotopic mouse models of human oral cancer. The presence of CD36+ metastasis-initiating cells correlates with a poor prognosis for numerous types of carcinomas. It is suggested that dietary lipids may promote metastasis (Pasqual, G, Avgustinova, A., Mejetta, S, Martin, M, Castellanos, A, Attolini, CS—O, Berenguer, A., Prats, N, Toll, A, Hueto, JA, Bescos, C, Di Croce, L, and Benitah, S A. 2017 “Targeting metastasis-initiating cells through the fatty acid receptor CD36” Nature 541:41-45).

In hepatocellular carcinoma exogenous palmitic acid activated an epithelial-mesenchymal transition (EMT)-like program and induced migration that was decreased by the CD36 inhibitor, sulpho-N-succinimidyl oleate (Nath, Aritro, et al. “Elevated free fatty acid uptake via CD36 promotes epithelial-mesenchymal transition in hepatocellular carcinoma.” Scientific reports 5 (2015). Body mass index was not associated with the degree of EMT highlighting that it is actually CD36 and free fatty acids that are important.

Cancer stems cells (CSCs) use CD36 to promote their maintenance. Oxidised phospholipids, ligands of CD36, were present in glioblastoma and the proliferation of CSCs but not non-CSCs increased with exposure to oxidised LDL. CD36 also correlated with patient prognosis.

### Chemotherapy Resistance

In addition to direct cytotoxic effects, chemotherapeutic agents harness the host immune system which contributes to anti-tumour activity. However, gemcitabine and 5-FU were shown to activate NLRP3 in myeloid-derived suppressor cells leading to production of IL-1β which curtails anti-tumour efficacy. Mechanistically these agents destabilised the lysosome to release cathepsin B to activate NLRP3. IL-1β drove the production of IL-17 from CD4+ T cells, which in turn blunted the efficacy of the chemotherapy. Higher anti-tumoral effects for both gemcitabine and 5-FU were observed when tumours were established in NLRP3−/− or Caps1−/− mice, or WT mice treated with IL-1RA. Myeloid-derived suppressor cell NLRP3 activation therefore limits the anti-tumour efficacy of gemcitabine and 5-FU (Bruchard, Melanie, et al. “Chemotherapy-triggered cathepsin B release in myeloid-derived suppressor cells activates the Nlrp3 inflammasome and promotes tumour growth.” Nature medicine 19.1 (2013): 57-64.). Compounds of the present disclosure may therefore be useful in chemotherapy to treat a range of cancers.

Compounds of the present disclosure, or pharmaceutically acceptable salts thereof, may be administered alone as a sole therapy or can be administered in addition with one or more other substances and/or treatments. Such conjoint treatment may be achieved by way of the simultaneous, sequential or separate administration of the individual components of the treatment.

For example, therapeutic effectiveness may be enhanced by administration of an adjuvant (i.e. by itself the adjuvant may only have minimal therapeutic benefit, but in combination with another therapeutic agent, the overall therapeutic benefit to the individual is enhanced). Alternatively, by way of example only, the benefit experienced by an individual may be increased by administering the compound of Formula (III) with another therapeutic agent (which also includes a therapeutic regimen) that also has therapeutic benefit.

In the instances where the compound of the present disclosure is administered in combination with other therapeutic agents, the compound of the disclosure need not be administered via the same route as other therapeutic agents, and may, because of different physical and chemical characteristics, be administered by a different route. For example, the compound of the disclosure may be administered orally to generate and maintain good blood levels thereof, while the other therapeutic agent may be administered intravenously. The initial administration may be made according to established protocols known in the art, and then, based upon the observed effects, the dosage, modes of administration and times of administration can be modified by the skilled clinician.

The particular choice of other therapeutic agent will depend upon the diagnosis of the attending physicians and their judgment of the condition of the individual and the appropriate treatment protocol. According to this aspect of the disclosure there is provided a combination for use in the treatment of a disease in which inflammasome activity is implicated comprising a compound of the disclosure as defined hereinbefore, or a pharmaceutically acceptable salt thereof, and another suitable agent.

According to a further aspect of the disclosure there is provided a pharmaceutical composition which comprises a compound of the disclosure, or a pharmaceutically acceptable salt thereof, in combination with a suitable, in association with a pharmaceutically acceptable diluent or carrier.

In addition to its use in therapeutic medicine, compounds of Formula (III) and pharmaceutically acceptable salts thereof are also useful as pharmacological tools in the development and standardisation of in vitro and in vivo test systems for the evaluation of the effects of inhibitors of inflammasome in laboratory animals such as dogs, rabbits, monkeys, rats and mice, as part of the search for new therapeutic agents.

In any of the above-mentioned pharmaceutical composition, process, method, use, medicament, and manufacturing features of the instant disclosure, any of the alternate embodiments of macromolecules of the present disclosure described herein also apply.

### Routes of Administration

The compounds of the disclosure or pharmaceutical compositions comprising these compounds may be administered to a subject by any convenient route of administration, whether systemically/peripherally or topically (i.e., at the site of desired action).

Routes of administration include, but are not limited to, oral (e.g. by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### Definitions

Unless otherwise stated, the following terms used in the specification and claims have the following meanings set out below.

Without wishing to be limited by this statement, it is understood that, while various options for variables are described herein, the disclosure intends to encompass operable embodiments having combinations of the options. The disclosure may be interpreted as excluding the non-operable embodiments caused by certain combinations of the options.

It is to be understood that a compound of the present disclosure may be depicted in a neutral form, a cationic form (e.g., carrying one or more positive charges), or an anionic form (e.g., carrying one or more negative charges), all of which are intended to be included in the scope of the present disclosure. For example, when a compound of the present disclosure is depicted in an anionic form, such depiction also refers to the various neutral forms, cationic forms, and anionic forms of the compound. For another example, when a compound the present disclosure is depicted in an anionic form, such depiction also refers to various salts (e.g., sodium salt) of the anionic form of the compound. In some embodiments, the amine of a compound of the present disclosure is protonated.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

As used herein, “alkyl”, “C₁, C₂, C₃, C₄, C₅ or C₆ alkyl” or “C₁-C₆ alkyl” is intended to include C₁, C₂, C₃, C₄, C₅ or C₆ straight chain (linear) saturated aliphatic hydrocarbon groups and C₃, C₄, C₅ or C₆ branched saturated aliphatic hydrocarbon groups. For example, C₁-C₆ alkyl is intends to include C₁, C₂, C₃, C₄, C₅ and C₆ alkyl groups. Examples of alkyl include, moieties having from one to six carbon atoms, such as, but not limited to, methyl (i.e., CH₃), ethyl (i.e., CH₂CH₃), n-propyl, i-propyl, n-butyl, s-butyl, t-butyl, n-pentyl, i-pentyl or n-hexyl. In some embodiments, a straight chain or branched alkyl has six or fewer carbon atoms (e.g., C₁-C₆ for straight chain, C₃-C₆ for branched chain), and in another embodiment, a straight chain or branched alkyl has four or fewer carbon atoms.

As used herein, the term “optionally substituted alkyl” refers to unsubstituted alkyl or alkyl having designated substituents replacing one or more hydrogen atoms on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, azido, heterocyclyl, alkylaryl, or an aromatic or heteroaromatic moiety.

As used herein, the term “alkenyl” includes unsaturated aliphatic groups analogous in length and possible substitution to the alkyls described above, but that contain at least one double bond. For example, the term “alkenyl” includes straight chain alkenyl groups (e.g., ethenyl, propenyl, butenyl, pentenyl, hexenyl, heptenyl, octenyl, nonenyl, decenyl), and branched alkenyl groups. In certain embodiments, a straight chain or branched alkenyl group has six or fewer carbon atoms in its backbone (e.g., C₂-C₆ for straight chain, C₃-C₆ for branched chain). The term “C₂-C₆” includes alkenyl groups containing two to six carbon atoms. The term “C₃-C₆” includes alkenyl groups containing three to six carbon atoms.

As used herein, the term “optionally substituted alkenyl” refers to unsubstituted alkenyl or alkenyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, heterocyclyl, alkylaryl, or an aromatic or heteroaromatic moiety.

As used herein, the term “alkynyl” includes unsaturated aliphatic groups analogous in length and possible substitution to the alkyls described above, but which contain at least one triple bond. For example, “alkynyl” includes straight chain alkynyl groups (e.g., ethynyl, propynyl, butynyl, pentynyl, hexynyl, heptynyl, octynyl, nonynyl, decynyl), and branched alkynyl groups. In certain embodiments, a straight chain or branched alkynyl group has six or fewer carbon atoms in its backbone (e.g., C₂-C₆ for straight chain, C₃-C₆ for branched chain). The term “C₂-C₆” includes alkynyl groups containing two to six carbon atoms. The term “C₃-C₆” includes alkynyl groups containing three to six carbon atoms. As used herein, “C₂-C₆ alkenylene linker” or “C₂-C₆ alkynylene linker” is intended to include C₂, C₃, C₄, C₅ or C₆ chain (linear or branched) divalent unsaturated aliphatic hydrocarbon groups. For example, C₂-C₆ alkenylene linker is intended to include C₂, C₃, C₄, C₅ and C₆ alkenylene linker groups.

As used herein, the term “optionally substituted alkynyl” refers to unsubstituted alkynyl or alkynyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulphhydryl, alkylthio, arylthio, thiocarboxylate, alkylsulphinyl, sulphonato, sulphamoyl, sulphonamido, nitro, trifluoromethyl, cyano, azido, heterocyclyl, alkylaryl, or an aromatic or heteroaromatic moiety.

Other optionally substituted moieties (such as optionally substituted cycloalkyl, heterocycloalkyl, aryl, or heteroaryl) include both the unsubstituted moieties and the moieties having one or more of the designated substituents. For example, substituted heterocycloalkyl includes those substituted with one or more alkyl groups, such as 2,2,6,6-tetramethyl-piperidinyl and 2,2,6,6-tetramethyl-1,2,3,6-tetrahydropyridinyl.

As used herein, the term “cycloalkyl” refers to a saturated or partially unsaturated hydrocarbon monocyclic or polycyclic (e.g., fused, bridged, or spiro rings) system having 3 to 30 carbon atoms (e.g., C₃-C₁₂, C₃-C₁₀, or C₃-C₈). Examples of cycloalkyl include, but are not limited to, cyclopropyl, cyclobutyl, cyclopentyl, cyclohexyl, cycloheptyl, cyclooctyl, cyclopentenyl, cyclohexenyl, cycloheptenyl, 1,2,3,4-tetrahydronaphthalenyl, and adamantyl. In the case of polycyclic cycloalkyl, only one of the rings in the cycloalkyl needs to be non-aromatic.

As used herein, the term “heterocycloalkyl” refers to a saturated or partially unsaturated 3-8 membered monocyclic, 6-12 membered bicyclic (fused, bridged, or spiro rings), or 11-14 membered tricyclic ring system (fused, bridged, or spiro rings) having one or more heteroatoms (such as O, N, S, P, or Se), e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulphur, unless specified otherwise. Examples of heterocycloalkyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, triazolidinyl, oxiranyl, azetidinyl, oxetanyl, thietanyl, 1,2,3,6-tetrahydropyridinyl, tetrahydropyranyl, dihydropyranyl, pyranyl, morpholinyl, tetrahydrothiopyranyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl, 1,4-dioxaspiro[4.5]decanyl, 1-oxaspiro[4.5]decanyl, 1-azaspiro[4.5]decanyl, 3′H-spiro[cyclohexane-1,1′-isobenzofuran]-yl, 7′H-spiro[cyclohexane-1,5′-furo[3,4-b]pyridin]-yl, 3′H-spiro[cyclohexane-1,1′-furo[3,4-c]pyridin]-yl, 3-azabicyclo[3.1.0]hexanyl, 3-azabicyclo[3.1.0]hexan-3-yl, 1,4,5,6-tetrahydropyrrolo[3,4-c]pyrazolyl, 3,4,5,6,7,8-hexahydropyrido[4,3-d]pyrimidinyl, 4,5,6,7-tetrahydro-1H-pyrazolo[3,4-c]pyridinyl, 5,6,7,8-tetrahydropyrido[4,3-d]pyrimidinyl, 2-azaspiro[3.3]heptanyl, 2-methyl-2-azaspiro[3.3]heptanyl, 2-azaspiro[3.5]nonanyl, 2-methyl-2-azaspiro[3.5]nonanyl, 2-azaspiro[4.5]decanyl, 2-methyl-2-azaspiro[4.5]decanyl, 2-oxa-azaspiro[3.4]octanyl, 2-oxa-azaspiro[3.4]octan-6-yl, and the like. In the case of multicyclic heterocycloalkyl, only one of the rings in the heterocycloalkyl needs to be non-aromatic.

As used herein, the term “aryl” includes groups with aromaticity, including “conjugated,” or multicyclic systems with one or more aromatic rings and do not contain any heteroatom in the ring structure. The term aryl includes both monovalent species and divalent species. Examples of aryl groups include, but are not limited to, phenyl, biphenyl, naphthyl and the like. Conveniently, an aryl is phenyl.

As used herein, the term “heteroaryl” is intended to include a stable 5-, 6-, or 7-membered monocyclic or 7-, 8-, 9-, 10-, 11- or 12-membered bicyclic aromatic heterocyclic ring which consists of carbon atoms and one or more heteroatoms, e.g., 1 or 1-2 or 1-3 or 1-4 or 1-5 or 1-6 heteroatoms, or, e.g., 1, 2, 3, 4, 5, or 6 heteroatoms, independently selected from the group consisting of nitrogen, oxygen and sulphur. The nitrogen atom may be substituted or unsubstituted (i.e., N or NR wherein R is H or other substituents, as defined). The nitrogen and sulphur heteroatoms may optionally be oxidised (i.e., N→O and S(O)ₚ, where p=1 or 2). It is to be noted that total number of S and O atoms in the aromatic heterocycle is not more than 1. Examples of heteroaryl groups include pyrrole, furan, thiophene, thiazole, isothiazole, imidazole, triazole, tetrazole, pyrazole, oxazole, isoxazole, pyridine, pyrazine, pyridazine, pyrimidine, and the like. Heteroaryl groups can also be fused or bridged with alicyclic or heterocyclic rings, which are not aromatic so as to form a multicyclic system (e.g., 4,5,6,7-tetrahydrobenzo[c]isoxazolyl).

Furthermore, the terms “aryl” and “heteroaryl” include multicyclic aryl and heteroaryl groups, e.g., tricyclic, bicyclic, e.g., naphthalene, benzoxazole, benzodioxazole, benzothiazole, benzoimidazole, benzothiophene, quinoline, isoquinoline, naphthyridine, indole, benzofuran, purine, deazapurine, indolizine.

The cycloalkyl, heterocycloalkyl, aryl, or heteroaryl ring can be substituted at one or more ring positions (e.g., the ring-forming carbon or heteroatom such as N) with such substituents as described above, for example, alkyl, alkenyl, alkynyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, azido, heterocyclyl, alkylaryl, or an aromatic or heteroaromatic moiety. Aryl and heteroaryl groups can also be fused or bridged with alicyclic or heterocyclic rings, which are not aromatic so as to form a multicyclic system (e.g., tetralin, methylenedioxyphenyl such as benzo[d][1,3]dioxole-5-yl). As used herein, the term “substituted,” means that any one or more hydrogen atoms on the designated atom is replaced with a selection from the indicated groups, provided that the designated atom's normal valency is not exceeded, and that the substitution results in a stable compound. When a substituent is oxo or keto (i.e., ═O), then 2 hydrogen atoms on the atom are replaced. Keto substituents are not present on aromatic moieties. Ring double bonds, as used herein, are double bonds that are formed between two adjacent ring atoms (e.g., C═C, C═N or N═N). “Stable compound” and “stable structure” are meant to indicate a compound that is sufficiently robust to survive isolation to a useful degree of purity from a reaction mixture, and formulation into an efficacious therapeutic agent.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

When any variable (e.g., R) occurs more than one time in any constituent or formula for a compound, its definition at each occurrence is independent of its definition at every other occurrence. Thus, for example, if a group is shown to be substituted with 0-2 R moieties, then the group may optionally be substituted with up to two R moieties and R at each occurrence is selected independently from the definition of R. Also, combinations of substituents and/or variables are permissible, but only if such combinations result in stable compounds.

As used herein, the term “hydroxy” or “hydroxyl” includes groups with an —OH or —O⁻.

As used herein, the term “halo” or “halogen” refers to fluoro, chloro, bromo and iodo.

The term “haloalkyl” or “haloalkoxyl” refers to an alkyl or alkoxyl substituted with one or more halogen atoms.

As used herein, the term “optionally substituted haloalkyl” refers to unsubstituted haloalkyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, azido, heterocyclyl, alkylaryl, or an aromatic or heteroaromatic moiety.

As used herein, the term “alkoxy” or “alkoxyl” includes substituted and unsubstituted alkyl, alkenyl and alkynyl groups covalently linked to an oxygen atom. Examples of alkoxy groups or alkoxyl radicals include, but are not limited to, methoxy, ethoxy, isopropyloxy, propoxy, butoxy and pentoxy groups. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino, and alkylarylamino), acylamino (including alkylcarbonylamino, arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy and trichloromethoxy.

As used herein, the expressions “one or more of A, B, or C,” “one or more A, B, or C,” “one or more of A, B, and C,” “one or more A, B, and C,” “selected from the group consisting of A, B, and C”, “selected from A, B, and C”, and the like are used interchangeably and all refer to a selection from a group consisting of A, B, and/or C, i.e., one or more As, one or more Bs, one or more Cs, or any combination thereof, unless indicated otherwise.

As used herein “Severe Acute Respiratory Syndrome Coronavirus 2 (SARS-CoV 2)” refers to the coronavirus that causes 2019 novel coronavirus disease (COVID-19). COVID-19 was first identified in 2019 in Wuhan, China, and has resulted in an ongoing global pandemic. As of August 2020, more than 25 million cases have been reported globally, resulting in an estimated 848,000 deaths. Common symptoms of COVID-19 include fever, cough, fatigue, shortness of breath, and loss of smell and taste. While many people have mild symptoms, some people develop acute respiratory distress syndrome, possibly caused by cytokine release syndrome (CRS), multi-organ failure, septic shock, and blood clots. Time from exposure to the virus to symptom onset is typically around 5 days but may range from 2 to 14 days.

As used herein “cytokine release syndrome (CRS)” refers to a systemic inflammatory response that can be triggered by a variety of factors, including but not limited to drugs, infections such as SARS-CoV 2, and immunotherapies such as chimeric antigen receptor T cell (CAR-T) therapies. In CRS, large numbers of immune cells (e.g., T cells) are activated and release inflammatory cytokines, which in turn activate additional immune cells. Symptoms include fever, fatigue, loss of appetite, muscle and joint pain, nausea, vomiting, diarrhea, rashes, respiratory insufficiency, low blood pressure, seizures, headache and confusion. CRS may respond to IL-6 receptor inhibition, and high doses of steroids.

As used herein, “adoptive cell therapy” refers to a form of treatment that uses immune cells to treat diseases such as cancer. Immune cells, for example T cells are collected from the subject or another source, grown in large numbers, and implanted into the subject to help the immune system fight the disease. Types of adoptive cell therapy include chimerica antigen receptor T cell (CAR-T) therapy, tumor infiltrating lymphocyte (TIL) therapy, and T cell receptor T cell (TCR-T) therapy.

The term “chimeric antigen receptors (CARs),” as used herein, may refer to artificial T-cell receptors, chimeric T-cell receptors, or chimeric immunoreceptors, for example, and encompass engineered receptors that graft an artificial specificity onto a particular immune effector cell. CARs may be employed to impart the specificity of a monoclonal antibody onto a T cell, thereby allowing a large number of specific T cells to be generated, for example, for use in adoptive cell therapy. For example, CARs may direct specificity of the cell expressing the CAR to a tumor associated antigen. In some embodiments, CARs comprise an intracellular activation domain, a transmembrane domain, and an extracellular domain comprising an antigen binding domain, and optionally an extracellular hinge. The antigen binding domain can be any antigen binding domain known in the art, including antigen binding domains derived from antibodies, Fab, F(ab′)2, nanobodies, single domain antigen binding domains, scFv, VHH, and the like. In particular aspects, CARs comprise fusions of single-chain variable fragments (scFv) derived from monoclonal antibodies, fused to a CD3 transmembrane domain and endodomain. In certain cases, CARs comprise domains for additional co-stimulatory signaling, such as CD3, FcR, CD27, CD28, CD137, DAP10, and/or 0X40.

A “T cell receptor (TCR)” is a protein complex found on the surface of T cells, or T lymphocytes, that is responsible for recognizing fragments of antigen as peptides bound to major histocompatibility complex (MHC) molecules. T cell receptors can be engineered to express antigen binding domains specific to particular antigens and used in the adoptive cell therapies described herein.

It is to be understood that the present disclosure provides methods for the synthesis of the compounds of any of the Formulae described herein. The present disclosure also provides detailed methods for the synthesis of various disclosed compounds of the present disclosure according to the following schemes as well as those shown in the Examples.

It is to be understood that, throughout the description, where compositions are described as having, including, or comprising specific components, it is contemplated those compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

It is to be understood that the synthetic processes of the disclosure can tolerate a wide variety of functional groups, therefore various substituted starting materials can be used. The processes generally provide the desired final compound at or near the end of the overall process, although it may be desirable in certain instances to further convert the compound to a pharmaceutically acceptable salt thereof.

It is to be understood that compounds of the present disclosure can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5^{th} edition, John Wiley & Sons: New York, 2001; Greene, T.W., Wuts, P.G.M., Protective Groups in Organic Synthesis, 3^{rd} edition, John Wiley & Sons: New York, 1999; R. Larock, Comprehensive Organic Transformations, VCH Publishers (1989); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), incorporated by reference herein, are useful and recognised reference textbooks of organic synthesis known to those in the art

One of ordinary skill in the art will note that, during the reaction sequences and synthetic schemes described herein, the order of certain steps may be changed, such as the introduction and removal of protecting groups. One of ordinary skill in the art will recognise that certain groups may require protection from the reaction conditions via the use of protecting groups. Protecting groups may also be used to differentiate similar functional groups in molecules. A list of protecting groups and how to introduce and remove these groups can be found in Greene, T.W., Wuts, P.G. M., Protective Groups in Organic Synthesis, 3^{rd} edition, John Wiley & Sons: New York, 1999.

It is to be understood that, unless otherwise stated, any description of a method of treatment includes use of the compounds to provide such treatment or prophylaxis as is described herein, as well as use of the compounds to prepare a medicament to treat or prevent such condition. The treatment includes treatment of human or non-human animals including rodents and other disease models.

As used herein, the term “subject” includes human and non-human animals, as well as cell lines, cell cultures, tissues, and organs. In some embodiments, the subject is a mammal. The mammal can be e.g., a human or appropriate non-human mammal, such as primate, mouse, rat, dog, cat, cow, horse, goat, camel, sheep or a pig. The subject can also be a bird or fowl. In some embodiments, the subject is a human.

As used herein, the term “subject in need thereof” refers to a subject having a disease or having an increased risk of developing the disease. A subject in need thereof can be one who has been previously diagnosed or identified as having a disease or disorder disclosed herein. A subject in need thereof can also be one who is suffering from a disease or disorder disclosed herein. Alternatively, a subject in need thereof can be one who has an increased risk of developing such disease or disorder relative to the population at large (i.e., a subject who is predisposed to developing such disorder relative to the population at large). A subject in need thereof can have a refractory or resistant a disease or disorder disclosed herein (i.e., a disease or disorder disclosed herein that does not respond or has not yet responded to treatment). The subject may be resistant at start of treatment or may become resistant during treatment. In some embodiments, the subject in need thereof received and failed all known effective therapies for a disease or disorder disclosed herein. In some embodiments, the subject in need thereof received at least one prior therapy.

As used herein, the term “treating” or “treat” describes the management and care of a patient for the purpose of combating a disease, condition, or disorder and includes the administration of a compound of the present disclosure, or a pharmaceutically acceptable salt, polymorph or solvate thereof, to alleviate the symptoms or complications of a disease, condition or disorder, or to eliminate the disease, condition or disorder. The term “treat” can also include treatment of a cell in vitro or an animal model.

It is to be understood that a compound of the present disclosure, or a pharmaceutically acceptable salt, polymorph or solvate thereof, can or may also be used to prevent a relevant disease, condition, or disorder, or used to identify suitable candidates for such purposes.

As used herein, the term “preventing,” “prevent,” or “protecting against” describes reducing or eliminating the onset of the symptoms or complications of such disease, condition or disorder.

It is to be understood that one skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3^{rd} edition), Cold Spring Harbor Press, Cold Spring Harbor, New York (2000); Coligan et al., Current Protocols in Immunology, John Wiley & Sons, N.Y.; Enna et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, PA, 18^{th} edition (1990). These texts can, of course, also be referred to in making or using an aspect of the disclosure.

It is to be understood that the present disclosure also provides pharmaceutical compositions comprising any compound described herein in combination with at least one pharmaceutically acceptable excipient or carrier.

As used herein, the term “pharmaceutical composition” is a formulation containing the compounds of the present disclosure in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches, and inhalants. In one embodiment, the active compound is mixed under sterile conditions with a pharmaceutically acceptable carrier, and with any preservatives, buffers, or propellants that are required.

As used herein, the term “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

It is to be understood that a pharmaceutical composition of the disclosure is formulated to be compatible with its intended route of administration. Examples of routes of administration include parenteral, e.g., intravenous, intradermal, subcutaneous, oral (e.g., ingestion), inhalation, transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulphite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

It is to be understood that a compound or pharmaceutical composition of the disclosure can be administered to a subject in many of the well-known methods currently used for chemotherapeutic treatment. For example, a compound of the disclosure may be injected into the blood stream or body cavities or taken orally or applied through the skin with patches. The dose chosen should be sufficient to constitute effective treatment but not so high as to cause unacceptable side effects. The state of the disease condition (e.g., a disease or disorder disclosed herein) and the health of the patient should preferably be closely monitored during and for a reasonable period after treatment.

As used herein, the term “therapeutically effective amount”, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

It is to be understood that, for any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing active compounds of the present disclosure may be manufactured in a manner that is generally known, e.g., by means of conventional mixing, dissolving, granulating, dragee-making, levigating, emulsifying, encapsulating, entrapping, or lyophilising processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilisation. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebuliser.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved.

In therapeutic applications, the dosages of the pharmaceutical compositions used in accordance with the disclosure vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the symptoms of the disease or disorder disclosed herein and also preferably causing complete regression of the disease or disorder. Dosages can range from about 0.01 mg/kg per day to about 5000 mg/kg per day. In preferred aspects, dosages can range from about 1 mg/kg per day to about 1000 mg/kg per day. In an aspect, the dose will be in the range of about 0.1 mg/day to about 50 g/day; about 0.1 mg/day to about 25 g/day; about 0.1 mg/day to about 10 g/day; about 0.1 mg to about 3 g/day; or about 0.1 mg to about 1 g/day, in single, divided, or continuous doses (which dose may be adjusted for the patient's weight in kg, body surface area in m², and age in years). An effective amount of a pharmaceutical agent is that which provides an objectively identifiable improvement as noted by the clinician or other qualified observer. Improvement in survival and growth indicates regression. As used herein, the term “dosage effective manner” refers to amount of an active compound to produce the desired biological effect in a subject or cell.

It is to be understood that the pharmaceutical compositions can be included in a container, pack, or dispenser together with instructions for administration.

It is to be understood that, for the compounds of the present disclosure being capable of further forming salts, all of these forms are also contemplated within the scope of the claimed disclosure.

As used herein, the term “pharmaceutically acceptable salts” refer to derivatives of the compounds of the present disclosure wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulphonic, acetic, ascorbic, benzene sulphonic, benzoic, bicarbonic, carbonic, citric, edetic, ethane disulphonic, 1,2-ethane sulphonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulphonic, maleic, malic, mandelic, methane sulphonic, napsylic, nitric, oxalic, pamoic, pantothenic, phenylacetic, phosphoric, polygalacturonic, propionic, salicylic, stearic, subacetic, succinic, sulphamic, sulphanilic, sulphuric, tannic, tartaric, toluene sulphonic, and the commonly occurring amine acids, e.g., glycine, alanine, phenylalanine, arginine, etc.

In some embodiments, the pharmaceutically acceptable salt is a sodium salt, a potassium salt, a calcium salt, a magnesium salt, a diethylamine salt, a choline salt, a meglumine salt, a benzathine salt, a tromethamine salt, an ammonia salt, an arginine salt, or a lysine salt.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulphonic acid, 2-naphthalenesulphonic acid, 4-toluenesulphonic acid, camphorsulphonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In the salt form, it is understood that the ratio of the compound to the cation or anion of the salt can be 1:1, or any ratio other than 1:1, e.g., 3:1, 2:1, 1:2, or 1:3.

It is to be understood that all references to pharmaceutically acceptable salts include solvent addition forms (solvates) or crystal forms (polymorphs) as defined herein, of the same salt.

The compounds, or pharmaceutically acceptable salts thereof, are administered orally, nasally, transdermally, pulmonary, inhalationally, buccally, sublingually, intraperitoneally, subcutaneously, intramuscularly, intravenously, rectally, intrapleurally, intrathecally, and parenterally. In one embodiment, the compound is administered orally. One skilled in the art will recognise the advantages of certain routes of administration.

The dosage regimen utilising the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

Techniques for formulation and administration of the disclosed compounds of the disclosure can be found in Remington: the Science and Practice of Pharmacy, 19^{th} edition, Mack Publishing Co., Easton, PA (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

In the synthetic schemes described herein, compounds may be drawn with one particular configuration for simplicity. Such particular configurations are not to be construed as limiting the disclosure to one or another isomer, tautomer, regioisomer or stereoisomer, nor does it exclude mixtures of isomers, tautomers, regioisomers or stereoisomers; however, it will be understood that a given isomer, tautomer, regioisomer or stereoisomer may have a higher level of activity than another isomer, tautomer, regioisomer or stereoisomer.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

As use herein, the phrase “compound of the disclosure” refers to those compounds which are disclosed herein, both generically and specifically.

### Exemplary Embodiments

Embodiment 1. A compound of Formula (III):

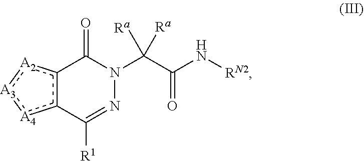

or a prodrug, solvate, or pharmaceutically acceptable salt thereof, wherein:
each [symbol] is independently a single bond or double bond as valency permits;
A₂ is CR², N, NR^{2a}, O, or S, as valency allows;
A₃ is CR², N, NR^{2a}, O, or S, as valency allows;
A₄ is CR², N, NR^{2a}, O, or S, as valency allows,
wherein at least one of A₂, A₃, or A₄ is N, NR^{2a}, O, or S, wherein when A₂ is S, A₄ is CR², NR^{2a}, O, or S;
R¹ is H, —N(C₁-C₆ alkyl)₂, C₁-C₆ alkyl, C₂-C₆ alkenyl, or C₃-C₁₂ cycloalkyl, wherein the —N(C₁-C₆ alkyl)₂, C₁-C₆ alkyl, C₂-C₆ alkenyl, or C₃-C₁₂ cycloalkyl is optionally substituted with one or more R^{1S};
each R^{1S} independently is halogen, cyano, —OH, or C₁-C₆ alkyl;
each R² independently is H, halogen, cyano, —OH, —NH₂, —NO₂, —C(═O)NH₂, C₁-C₆ alkyl, C₂-C₆ alkenyl, C₂-C₆ alkynyl, —O(C₁-C₆ alkyl), —NH(C₁-C₆ alkyl), —N(C₁-C₆ alkyl)₂, C₃-C₁₂ cycloalkyl, 3- to 12-membered heterocycloalkyl, C₆-C₁₀ aryl, or 5- to 10-membered heteroaryl, wherein the C₁-C₆ alkyl, C₂-C₆ alkenyl, C₂-C₆ alkynyl, —O(C₁-C₆ alkyl), —NH(C₁-C₆ alkyl), —N(C₁-C₆ alkyl)₂, C₃-C₁₂ cycloalkyl, 3- to 12-membered heterocycloalkyl, C₆-C₁₀ aryl, or 5- to 10-membered heteroaryl is optionally substituted with one or more R^{2S},
or two R² together with the atoms to which they are attached form a C₃-C₁₂ cycloalkyl or 3- to 12-membered heterocycloalkyl, wherein the C₃-C₁₂ cycloalkyl or 3- to 12-membered heterocycloalkyl is optionally substituted with one or more R^{2S};
each R^{2S} independently is halogen, —OH, —O(C₁-C₆ alkyl), —NH₂, —NH(C₁-C₆ alkyl), —N(C₁-C₆ alkyl)₂, or C₃-C₁₂ cycloalkyl;
each R^{2a} independently is H, C₁-C₆ alkyl, C₂-C₆ alkenyl, C₂-C₆ alkynyl, C₁-C₆ haloalkyl, —(CH₂)₀₋₃—C₃-C₁₂ cycloalkyl, or —(CH₂)₀₋₃-(3- to 12-membered heterocycloalkyl);
each R^{a} independently is H or C₁-C₆ alkyl; or two R^{a}, together with the atom they attach to, form C₃-C₁₂ cycloalkyl;
R^{N2} is C₁-C₆ alkyl, C₂-C₆ alkenyl, C₂-C₆ alkynyl, —O—(C₁-C₆ alkyl), —O—(C₂-C₆ alkenyl), —O—(C₂-C₆ alkynyl), —NH—(C₁-C₆ alkyl), —NH—(C₂-C₆ alkenyl), —NH—(C₂-C₆ alkynyl), C₃-C₁₂ cycloalkyl, 3- to 12-membered heterocycloalkyl, C₆-C₁₀ aryl, 5- to 10-membered heteroaryl, —(C₁-C₆ alkyl)-(C₃-C₁₂ cycloalkyl), —(C₁-C₆ alkyl)-(3- to 12-membered heterocycloalkyl), —(C₁-C₆ alkyl)-(C₆-C₁₀ aryl), or —(C₁-C₆ alkyl)-(5- to 10-membered heteroaryl); wherein the C₁-C₆ alkyl, C₂-C₆ alkenyl, C₂-C₆ alkynyl, —O—(C₁-C₆ alkyl), —O—(C₂-C₆ alkenyl), —O—(C₂-C₆ alkynyl), —NH—(C₁-C₆ alkyl), —NH—(C₂-C₆ alkenyl), —NH—(C₂-C₆ alkynyl), C₃-C₁₂ cycloalkyl, 3- to 12-membered heterocycloalkyl, C₆-C₁₀ aryl, 5- to 10-membered heteroaryl, —(C₁-C₆ alkyl)-(C₃-C₁₂ cycloalkyl), —(C₁-C₆ alkyl)-(3- to 12-membered heterocycloalkyl), —(C₁-C₆ alkyl)-(C₆-C₁₀ aryl), or —(C₁-C₆ alkyl)-(5- to 10-membered heteroaryl) is optionally substituted with one or more R^{N2a};
each R^{N2a} independently is oxo, halogen, cyano, —OH, —NH₂, —C(═O)H, —C(═O)OH, C₁-C₆ alkyl, C₂-C₆ alkenyl, C₂-C₆ alkynyl, —O(C₁-C₆ alkyl), —NH(C₁-C₆ alkyl), —N(C₁-C₆ alkyl)₂, —C(═O)(C₁-C₆ alkyl), —C(═O)O(C₁-C₆ alkyl), —NHC(═O)O(C₁-C₆ alkyl), —S(═O)₂(C₁-C₆ alkyl), —S(═O)₂N(C₁-C₆ alkyl)₂, C₃-C₁₂ cycloalkyl, 3- to 12-membered heterocycloalkyl, C₆-C₁₀ aryl, 5- to 10-membered heteroaryl, —(C₁-C₆ alkyl)-(C₃-C₁₂ cycloalkyl), —(C₁-C₆ alkyl)-(3- to 12-membered heterocycloalkyl), —(C₁-C₆ alkyl)-(C₆-C₁₀ aryl), or —(C₁-C₆ alkyl)-(5- to 10-membered heteroaryl); wherein the C₁-C₆ alkyl, C₂-C₆ alkenyl, C₂-C₆ alkynyl, —O(C₁-C₆ alkyl), —NH(C₁-C₆ alkyl), —N(C₁-C₆ alkyl)₂, —C(═O)(C₁-C₆ alkyl), —C(═O)O(C₁-C₆ alkyl), —NHC(═O)O(C₁-C₆ alkyl), —S(═O)₂(C₁-C₆ alkyl), —S(═O)₂N(C₁-C₆ alkyl)₂, C₃-C₁₂ cycloalkyl, 3- to 12-membered heterocycloalkyl, C₆-C₁₀ aryl, 5- to 10-membered heteroaryl, —(C₁-C₆ alkyl)-(C₃-C₁₂ cycloalkyl), —(C₁-C₆ alkyl)-(3- to 12-membered heterocycloalkyl), —(C₁-C₆ alkyl)-(C₆-C₁₀ aryl), or —(C₁-C₆ alkyl)-(5- to 10-membered heteroaryl) is optionally substituted with one or more R^{N2ab}; and
each R^{N2ab} independently is oxo, halogen, cyano, —OH, —NH₂, —C(═O)H, —C(═O)OH, —O(C₁-C₆ alkyl), —NH(C₁-C₆ alkyl), —N(C₁-C₆ alkyl)₂, —C(═O)(C₁-C₆ alkyl), —C(═O)O(C₁-C₆ alkyl), —NHC(═O)O(C₁-C₆ alkyl), —S(═O)₂(C₁-C₆ alkyl), or —S(═O)₂N(C₁-C₆ alkyl)₂.

Embodiment 2. The compound of Embodiment 1, wherein:
R¹ is C₁-C₆ alkyl, C₂-C₆ alkenyl, or C₃-C₇ cycloalkyl, wherein the C₁-C₆ alkyl is optionally substituted with one or more R^{1S};
each R^{1S} independently is halogen;
each R² independently is H, halogen, cyano, —NH₂, C₁-C₆ alkyl, —O(C₁-C₆ alkyl), —NH(C₁-C₆ alkyl), —N(C₁-C₆ alkyl)₂, C₃-C₁₂ cycloalkyl, wherein the C₁-C₆ alkyl and —NH(C₁-C₆ alkyl) is optionally substituted with one or more R^{2S},
each R^{2S} independently is halogen, —O(C₁-C₆ alkyl or —NH₂;
each R^{2a} independently is C₁-C₆ alkyl or —(CH₂)₀₋₃—C₃-C₁₂ cycloalkyl;
R^{N2} is C₃-C₁₂ cycloalkyl, 3- to 12-membered heterocycloalkyl or 5- to 10-membered heteroaryl, wherein the C₃-C₁₂ cycloalkyl, 3- to 12-membered heterocycloalkyl or 5- to 10-membered heteroaryl is optionally substituted with one or more R^{N2a}; and
each R^{N2a} independently is halogen, cyano, —OH, C₁-C₆ alkyl, C₃-C₁₂ cycloalkyl, —C(═O)O(C₁-C₆ alkyl), wherein the C₁-C₆ alkyl is optionally substituted with one or more R^{N2ab}; and
each R^{N2ab} independently is —C(═O)O(C₁-C₆ alkyl).

Embodiment 3. The compound of Embodiment 1 or Embodiment 2, wherein:
A₂ is S, A₃ is CR², and A₄ is CR²; or
A₂ is CR², A₃ is CR², and A₄ is S; or
A₂ is N, A₃ is NR^{2a}, and A₄ is CR²; or
A₂ is O, A₃ is CR², and A₄ is CR²; or
A₂ is NR^{2a}, A₃ is N, and A₄ is CR²; or
A₂ is N, A₃ is CR², and A₄ is NR^{2a}; or
A₂ is CR², A₃ is N, and A₄ is NR^{2a}; or
A₂ is CR², A₃ is NR^{2a}, and A₄ is N; or
A₂ is CR², A₃ is CR², and A₄ is O.

Embodiment 4. The compound of any one of the preceding Embodiments, wherein R² independently is H, halogen, cyano, —NH₂, C₁-C₆ alkyl —O(C₁-C₆ alkyl), —NH(C₁-C₆ alkyl), —N(C₁-C₆ alkyl)₂, C₃-C₁₂ cycloalkyl, wherein the C₁-C₆ alkyl and —NH(C₁-C₆ alkyl) is optionally substituted with one or more R^{2S}.

Embodiment 5. The compound of any one of the preceding Embodiments, wherein R² independently is H, halogen (such as chlorine or bromine), cyano, —NH₂, C₁-C₆ alkyl (such as methyl, ethyl, or propyl), —O(C₁-C₆ alkyl) (such as —O-methyl or —O-ethyl), —NH(C₁-C₆ alkyl) (such as —NH-methyl, —NH-CD₃, —NH-ethyl, or —NH-isopropyl), —N(C₁-C₆ alkyl)₂ (such as (—N(Me)(Et)), C₃-C₁₂ cycloalkyl (such as cyclopropyl), wherein the C₁-C₆ alkyl and —NH(C₁-C₆ alkyl) is optionally substituted with one or more R^{2s} (such as —CH₂—CF₃, —NHCH₂CHF₂, —CH₂—O-methyl, —NHCH₂CH₂OMe or —CH₂—NH₂).

Embodiment 6. The compound of any one of Embodiments 1 to 4, wherein R² independently is H, chlorine, bromine, cyano, —NH₂, methyl, ethyl, propyl, —O-methyl, —O— ethyl, —NH-methyl, —NH-CD₃, —NH-ethyl, —NH-isopropyl, —N(Me)(Et), cyclopropyl, —CH₂—CF₃, —NHCH₂CHF₂, —CH₂—O-methyl, —NHCH₂CH₂OMe or —CH₂—NH₂.

Embodiment 7. The compound of any one of the preceding Embodiments, wherein each R^{2a} independently is C₁-C₆ alkyl or —(CH₂)₀₋₃—C₃-C₁₂ cycloalkyl.

Embodiment 8. The compound of any one of Embodiments 1 to 6, wherein each R^{2a} independently is C₁-C₆ alkyl (such as methyl, ethyl, isopropyl) or —(CH₂)₀₋₃—C₃-C₁₂ cycloalkyl (such as cyclopropyl or cyclobutyl).

Embodiment 9. The compound of any one of Embodiments 1 to 6, wherein each R^{2a} independently is methyl, ethyl, isopropyl, cyclopropyl or cyclobutyl.

Embodiment 10. The compound of any one of the preceding Embodiments, wherein R¹ is C₁-C₆ alkyl, C₂-C₆ alkenyl or C₃-C₇ cycloalkyl, wherein C₁-C₆ alkyl is optionally substituted with one or more R^{1S}.

Embodiment 11. The compound of any one of Embodiments 1 to 9, wherein R¹ is C₁-C₆ alkyl (such as methyl, ethyl or isopropyl), C₂-C₆ alkenyl (such as isopropenyl), C₃-C₇ cycloalkyl (such as cyclopropyl) or C₆ alkyl is optionally substituted with one or more R^{1S} (such as fluoromethyl).

Embodiment 12. The compound of any one of Embodiments 1 to 9, wherein R¹ is methyl, ethyl, isopropyl, isopropenyl, cyclopropyl or fluoromethyl.

Embodiment 13. The compound of any one of the preceding Embodiments, wherein both R^{a} are H or two R^{a}, together with the atom they attach to, form C₃-C₁₂ cycloalkyl.

Embodiment 14. The compound of any one of Embodiments 1 to 12, wherein both R^{a} are H or two R^{a}, together with the atom they attach to, form C₃-C₇ cycloalkyl.

Embodiment 15. The compound of any one of Embodiments 1 to 12, wherein both R^{a} are H or two R^{a}, together with the atom they attach to, form, C₃-C₆ cycloalkyl.

Embodiment 16. The compound of any one of Embodiments 1 to 12, wherein both R^{a} are H or two R^{a}, together with the atom they attach to, form, cyclopropyl.

Embodiment 17. The compound of any one of the preceding Embodiments, wherein R^{N2} is C₃-C₁₂ cycloalkyl (such as cyclobutyl), 3- to 12-membered heterocycloalkyl (such as piperidinyl, octahydroindolizin-8-yl, or oxaspiro[3.3]heptan-6-yl) or 5- to 10-membered heteroaryl (such as oxazolyl, pyrimidinyl or triazolylpyridinyl), wherein the C₃-C₁₂ cycloalkyl, 3- to 12-membered heterocycloalkyl or 5- to 10-membered heteroaryl is optionally substituted with one or more R^{N2a}.

Embodiment 18. The compound of any one of Embodiments 1 to 16, wherein R^{N2} is cyclobutyl, piperidinyl, octahydroindolizin-8-yl, oxaspiro[3.3]heptan-6-yl), oxazolyl, pyrimidinyl or triazolylpyridinyl, each of which is optionally substituted with one or more R^{N2a}

Embodiment 19. The compound of any one of the preceding Embodiments, wherein R^{N2a} independently is halogen (such as F or Cl), cyano, —OH, C₁-C₆ alkyl (such as methyl), C₃-C₁₂ cycloalkyl (such as cyclopropyl or cyclobutyl), —C(═O)O(C₁-C₆ alkyl) (such as —COO— ethyl), wherein the C₁-C₆ alkyl (such as methyl) is optionally substituted with one or more R^{N2ab} (such as —C(═O)O(C₁-C₆ alkyl), in particular —C(═O)O(ethyl)).

Embodiment 20. The compound of any one of the preceding Embodiments, which is a compound of Formula (III-a), (III-b), (III-c), (III-d), (III-e), (III-f), or (III-g):

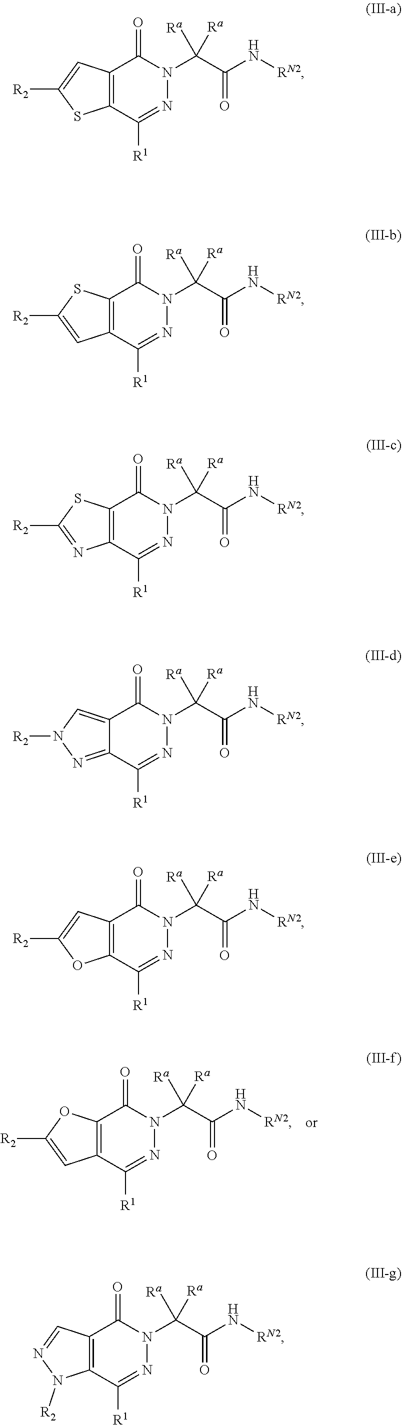

or a prodrug, solvate, or pharmaceutically acceptable salt thereof.

Embodiment 21. The compound of any one of Embodiments 1 to 19, which is a compound of Formula (III-b), Formula (III-d), or Formula (III-e).

Embodiment 22. The compound of any one of Embodiments 1 to 19, which is a compound of Formula (III-e).

Embodiment 23. The compound of any one of the preceding Embodiments, wherein the compound is selected from the compounds described in Table 1 and prodrugs and pharmaceutically acceptable salts thereof.

Embodiment 24. A compound being an isotopic derivative of the compound of any one of the preceding Embodiments.

Embodiment 25. A process for preparing a compound of Formula (III) of any one of the preceding Embodiments which comprises:

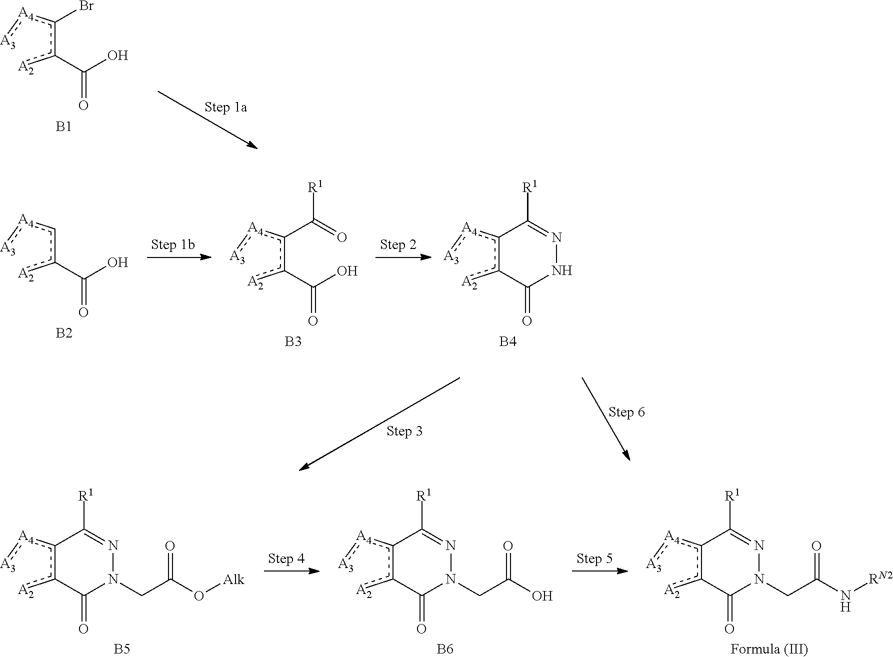

Embodiment 26. A pharmaceutical composition comprising the compound of any one of Embodiments 1 to 24 and a pharmaceutically acceptable diluent or carrier.

Embodiment 27. A method of inhibiting inflammasome activity, comprising contacting a cell with an effective amount of the compound of any one of Embodiments 1 to 24; optionally, the inflammasome is NLRP3 inflammasome, and the activity is in vitro or in vivo.

Embodiment 28. A method of treating or preventing a disease or disorder in a subject in need thereof, comprising administering to the subject the compound of any one of Embodiments 1 to 24, or the pharmaceutical composition of Embodiment 26.

Embodiment 29. The compound of any one of Embodiments 1 to 24 or the pharmaceutical composition of Embodiment 26, for use in inhibiting inflammasome activity; optionally, wherein the inflammasome is NLRP3 inflammasome, and the activity is in vitro or in vivo.

Embodiment 30. The compound of any one of Embodiments 1 to 24 or the pharmaceutical composition of Embodiment 26, for use in treating or preventing a disease or disorder.

Embodiment 31. Use of the compound of any one of Embodiments 1 to 24 in the manufacture of a medicament for inhibiting inflammasome activity; optionally, the inflammasome is NLRP3 inflammasome, and the activity is in vitro or in vivo.

Embodiment 32. Use of the compound of any one of Embodiments 1 to 24 in the manufacture of a medicament for treating or preventing a disease or disorder.

Embodiment 33. The method, compound for use, pharmaceutical composition, or use of any one of Embodiments 27 to 32, wherein the disease or disorder is associated with an implicated inflammasome activity; optionally, the disease or disorder is a disease or disorder in which inflammasome activity is implicated.

Embodiment 34. The method, compound for use, pharmaceutical composition, or use of any one of Embodiments 27 to 32, wherein the disease or disorder is an inflammatory disorder, an autoinflammatory disorder, an autoimmune disorder, a neurodegenerative disease, or cancer.

Embodiment 35. The method, compound for use, pharmaceutical composition, or use of any one of Embodiments 27 to 34, wherein the disease or disorder is an inflammatory disorder, an autoinflammatory disorder or an autoimmune disorder; optionally, the disease or disorder is selected from cryopyrin-associated auto-inflammatory syndrome (CAPS; e.g., familial cold autoinflammatory syndrome (FCAS), Muckle-Wells syndrome (MWS), chronic infantile neurological cutaneous and articular (CINCA) syndrome/neonatal-onset multisystem inflammatory disease (NOMID)), familial Mediterranean fever (FMF), nonalcoholic fatty liver disease (NAFLD), non-alcoholic steatohepatitis (NASH), gout, rheumatoid arthritis, osteoarthritis, Crohn's disease, chronic obstructive pulmonary disease (COPD), chronic kidney disease (CKD), fibrosis, obesity, type 2 diabetes, multiple sclerosis, dermatological disease (e.g., acne) and neuroinflammation occurring in protein misfolding diseases (e.g., Prion diseases).

Embodiment 36. The method, compound for use, pharmaceutical composition, or use of any one of Embodiments 27 to 34, wherein the disease or disorder is a neurodegenerative disease; optionally, the disease or disorder is Parkinson's disease or Alzheimer's disease.

Embodiment 37. The method, compound for use, pharmaceutical composition, or use of any one of Embodiments 27 to 34, wherein the disease or disorder is cancer; optionally, the cancer is metastasizing cancer, brain cancer, gastrointestinal cancer, skin cancer, non-small-cell lung carcinoma, head and neck squamous cell carcinoma or colorectal adenocarcinoma.

Embodiment 38. The method, compound for use, pharmaceutical composition, or use of any one of Embodiments 27 to 34, wherein the disease or disorder is an inflammatory disease.

Embodiment 39. The method, compound for use, pharmaceutical composition, or use of Embodiment 38, wherein the inflammatory disease is associated with an infection.

Embodiment 40. The method, compound for use, pharmaceutical composition, or use of Embodiment 39, wherein the infection is a viral infection.

Embodiment 41. The method, compound for use, pharmaceutical composition, or use of Embodiment 40, wherein the viral infection is caused by a single stranded RNA virus.

Embodiment 42. The method, compound for use, pharmaceutical composition, or use of Embodiment 41, wherein the single stranded RNA virus is a coronavirus.

Embodiment 43. The method, compound for use, pharmaceutical composition, or use of Embodiment 42, wherein the coronavirus is Severe Acute Respiratory Syndrome Coronavirus 2 (SARS-CoV 2).

Embodiment 44. The method, compound for use, pharmaceutical composition, or use of Embodiment 38, wherein the inflammatory disease is associated with an infection by SARS-CoV 2 leading to 2019 novel coronavirus disease (COVID-19).

Embodiment 45. The method, compound for use, pharmaceutical composition or use of Embodiment 38, wherein the inflammatory disease comprises cytokine release syndrome (CRS).

Embodiment 46. The method, compound for use, pharmaceutical composition, or use of Embodiment 45, wherein the CRS is associated with COVID-19.

Embodiment 47. The method, compound for use, pharmaceutical composition, or use of Embodiment 45, wherein the CRS is associated with an adoptive cell therapy.

Embodiment 48. The method, compound for use, pharmaceutical composition, or use of Embodiment 47, wherein the adoptive cell therapy comprises chimeric antigen receptor T cell (CAR-T) therapy.

### EXAMPLES

For exemplary purpose, salts of the compounds of Formula (III) are synthesized and tested in the examples. It is understood that neutral compounds of Formula (III) may be similarly synthesized and tested using the exemplary procedures described in the examples. Further, it is understood that the salts (e.g., sodium salt) of the compounds of Formula (III) may be converted to the corresponding neutral compounds using routine techniques in the art (e.g., pH adjustment and, optionally, extraction (e.g., into an aqueous phase)).

### Synthesis of Compounds

Compounds of Formula (III) can be prepared using the methods detailed herein. Those skilled in the art may be able to envisage alternative synthetic routes, using a variety of starting materials and reagents to prepare the disclosed compounds of Formula (III) and to make further modifications. For exemplary purpose, salts of some of the compounds of Formula (III) are synthesized and tested in the examples. It is understood that neutral compounds of Formula (III) may be similarly synthesized and tested using the exemplary procedures described in the examples. Further, it is understood that the salts (e.g., hydrochloride salt) of the compounds of Formula (III) may be converted to the corresponding neutral compounds using routine techniques in the art (e.g., pH adjustment and, optionally, extraction (e.g., into an aqueous phase)).

¹H, ¹³C and ¹⁹F Nuclear magnetic resonance (NMR) spectra were recorded at 400 MHz or 300 MHz as stated and at 300.3 K unless otherwise stated; the chemical shifts (δ) are reported in parts per million (ppm), relative to the residual solvent peak and the multiplicity reported together with the associated coupling constant (J), where applicable. Spectra were recorded using a Bruker or Varian instrument with 8, 16, 32 or 64 scans.

LC-MS chromatograms and spectra were recorded using an Agilent 1200 or Shimadzu LC-20 AD&MS 2020 instrument using a C-18 column such as a Luna-C18 2.0×30 mm or Xbridge Shield RPC18 2.1×50 mm. Injection volumes were 0.7-8.0 μL and the flow rates were typically 0.8 or 1.2 mL/min. Detection methods were diode array (DAD) or evaporative light scattering (ELSD) as well as positive ion electrospray ionisation. MS range was 100-1000 Da. Solvents were gradients of water and acetonitrile both containing a modifier (typically 0.01-0.04%) such as trifluoroacetic acid or ammonium carbonate.

UPLC-MS analysis was carried out on a Waters Acquity UPLC system consisting of an Acquity I-Class Sample Manager-FL, Acquity I-Class Binary Solvent Manager and an Acquity UPLC Column Manager. UV detection was afforded using an Acquity UPLC PDA detector (scanning from 210 to 400 nm), whilst mass detection was achieved using an Acquity QDa detector (mass scanning from 100-1250 Da; positive and negative modes simultaneously), and ELS detection was achieved using an Acquity UPLC ELS Detector. A Waters Acquity UPLC BEH C18 column (2.1×50 mm, 1.7 mm) was used to separate the analytes.

Samples were typically prepared by dissolution (with or without sonication) into 1 mL of 50% (v/v) MeCN in water. The resulting solutions were then filtered through a 0.2 mm syringe filter before submitting for analysis. All of the solvents, including formic acid and 36% ammonia solution, were purchased as HPLC grade. Solvents were gradients of water and acetonitrile both containing a modifier (typically 0.01-0.04%) such as formic acid or ammonia.

### ABBREVIATIONS

Ac Acetate
ACN, MeCN Acetonitrile
AcOH Acetic acid
CAN Ceric ammonium nitrate
CDCl3 Chloroform-d
DCM Dichloromethane
DIPEA N,N-Diisopropylethylamine
DME Dimethyl ether
DMEDA 1,2-Dimethylethylenediamine
DMF N,N-Dimethylformamide
DMSO-d₆ Dimethyl sulfoxide-d₆
dppf 1,1′-Bis(diphenylphosphino)ferrocene
dtbbpy 4,4′-Di-tert-butyl-2,2′-bipyridine
eq. Equivalents
ESI Electrospray Ionisation
EtOAc Ethyl Acetate
EtOH Ethanol
FCC Flash column chromatography
h Hour(s)
HATU (1-[Bis(dimethylamino)methylene]-1H-1,2,3-triazolo[4,5-b]pyridinium 3-oxide hexafluorophosphate, Hexafluorophosphate Azabenzotriazole Tetramethyl Uronium
HPLC High-performance Liquid Chromatography
LCMS Liquid Chromatography-Mass Spectrometry
LiHMDS Lithium bis(trimethylsilyl)amide
MeOH-d₄ Deuterated methanol
MeOH Methanol
min Minute(s)
n-BuLi n-Butyllithium
MTBE Methyl tert-butylether
NMR Nuclear Magnetic Resonance
RM Reaction mixture
RP Reverse phase
rt Room temperature
s-Phos 2-Dicyclohexylphosphino-2′,6′-dimethoxybiphenyl
TBHP Tert-butyl hydroperoxide
TEA Triethylamine
TFA Trifluoroacetic acid
THF Tetrahydrofuran
TMS Trimethylsilyl
TTMSS Tris-(trimethylsilyl)silane,
Y Yield
   General routes for the preparation of a compound of the present disclosure are described in Schemes 1-3.

### Intermediate A1. Tert-butyl (3R)-3-[(2-chloroacetyl) amino]piperidine-1-carboxylate

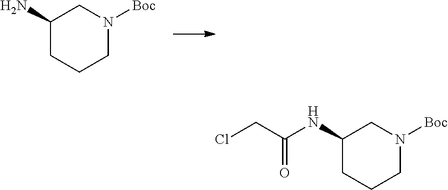 [SMILES: CC(C)(C)OC(=O)N1CCC[C@@H](N)C1 ; CC(C)(C)OC(=O)N1CCC[C@@H](NC(=O)CCl)C1]

To a mixture of tert-butyl (3R)-3-aminopiperidine-1-carboxylate (10 g, 49.9 mmol) in THE (150 mL) at 25° C. was added TEA (10.4 mL, 74.9 mmol). The solution was stirred for 30 min, then 2-chloroacetyl chloride (3.97 mL, 49.93 mmol) was added. The RM was stirred for 1 h, diluted with water (100 mL) and extracted with ethyl acetate (3×150 mL). The combined organic phases were dried over Na₂SO₄, filtered and the filtrate concentrated under reduced pressure to give the title compound as a solid (Y=98%). ¹H NMR (400 MHz, DMSO-d₆) δ 8.14 (d, J=7 Hz, 1H), 4.04 (s, 2H), 3.85-3.50 (m, 3H), 3.00-2.57 (m, 2H), 1.83-1.72 (m, 1H), 1.71-1.61 (m, 1H), 1.46-1.24 (m, 11H). LCMS (ESI): m/z: [M-55]f=221.0.

### Intermediate A2. 2-Chloro-N-[(3R)-1-cyclopropyl-3-piperidyl]acetamide

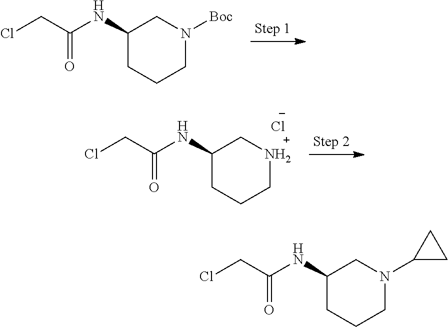 [SMILES: CC(C)(C)OC(=O)N1CCC[C@@H](NC(=O)CCl)C1 ; Cl ; O=C(CCl)N[C@@H]1CCCNC1 ; O=C(CCl)N[C@@H]1CCCN(C2CC2)C1]

Step 1. 2-Chloro-N-[(3R)-3-piperidyl]acetamide hydrochloride. Tert-butyl (3R)-3-[(2-chloroacetyl) amino]piperidine-1-carboxylate (1.5 g, 5.42 mmol) was stirred in 4 N HCl in EtOAc (50 mL) at 25° C. for 1 h. The RM was concentrated in vacuo to give the title compound as a yellow solid (quantitative), which was used without purification.

Step 2. Tert-butyl (3R)-3-[(2-chloroacetyl) amino]piperidine-1-carboxylate. To a mixture of 2-chloro-N-[(3R)-3-piperidyl]acetamide hydrochloride (1.16 g, 5.44 mmol) and (1-ethoxycyclopropoxy)-trimethyl-silane (6.02 mL, 29.9 mmol) in MeOH (10 mL) was added AcOH (0.25 mL, 4.35 mmol) and NaBH₃CN (1.37 g, 21.8 mmol). The reaction was stirred at 45° C. for 8 h. The reaction was conducted three times in total, and the batches were combined and concentrated in vacuo. The residue was diluted with water (10 mL) and extracted with ethyl acetate (3×10 mL). The combined organic phases were washed with brine (3×10 mL), dried with anhydrous Na₂SO₄, filtered and concentrated in vacuo to give the title compound as a yellow solid, which was used for the next reaction without purification. (5.4 g, crude).

### Intermediate A3. 2-Chloro-N-[(3R)-1-methyl-3-piperidyl]acetamide

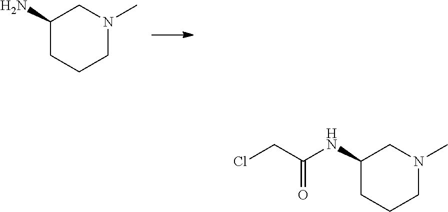

To a solution of (3R)-1-methylpiperidin-3-amine (1.0 g, 8.76 mmol) in DCM (10 mL) at 25° C. was added dropwise TEA (1.83 mL, 13.1 mmol) and 2-chloroacetyl chloride (836 μL, 10.5 mmol). The mixture was stirred at 25° C. for 1 h, diluted with water (10 mL) and extracted with DCM (3×10 mL). The organic layers were combined and concentrated under reduced pressure. The resulting residue was purified by prep-HPLC (column: Phenomenex Luna C18, 80×40 mm, 3 μm; mobile phase: [water (0.04% HCl)−ACN]; B: 1-5%, 4 min). ACN was removed from the fractions under reduced pressure. The aqueous phase was basified to pH 8 with saturated aqueous Na₂CO₃ solution and the solution was extracted with DCM (3×10 mL). The combined organic layers were washed with brine (10 mL), dried over Na₂SO₄ and concentrated to give the title compound as a yellow oil. Y=60%.

### Intermediate A4

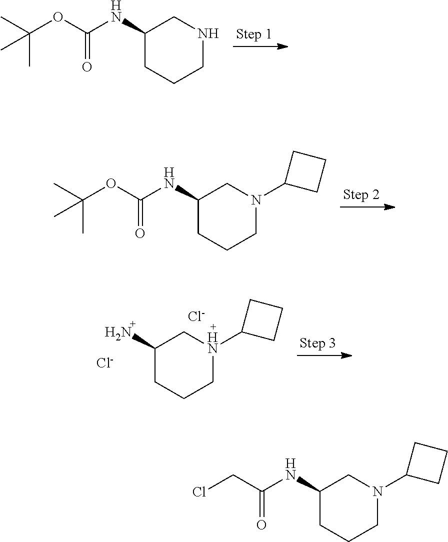

Step 1. Tert-butyl N-[(3R)-1-cyclobutyl-3-piperidyl]carbamate. To a mixture of tert-butyl N-[(3R)-3-piperidyl]carbamate (100 g, 499 mmol) and cyclobutanone (48.5 mL, 649 mmol) in DCE (500 mL) at 25° C. was added AcOH (71.4 mL, 1.25 mol). After stirring for 30 min, NaBH(OAc)₃ (265 g, 1.25 mol) was added and the mixture was stirred at 25° C. for 1 h. Saturated aqueous Na₂CO₃ (500 mL) was added and the resulting mixture was stirred for 5 min. The solution was extracted with DCM (3×300 mL). The combined organic phase was washed with brine (300 mL), dried over anhydrous Na₂SO₄, filtered and concentrated under vacuum to give the title compound as a yellow solid. Y=95%.

Step 2. (3R)-1-Cyclobutylpiperidin-3-amine hydrochloride. Tert-butyl N-[(3R)-1-cyclobutyl-3-piperidyl]carbamate (120 g, 472 mmol) was stirred in 4 N HCl in EtOAc (1 L) at 25° C. for 30 min. The reaction was concentrated under vacuum to give the title compound as an oil. This was used without further purification.

Step 3. 2-Chloro-N-[(3R)-1-cyclobutyl-3-piperidyl]acetamide. To a mixture of (3R)-1-cyclobutylpiperidin-3-amine hydrochloride (90 g, 1.42 mol) in THE (1 L) at 25° C. was added TEA (197 mL, 1.42 mol) and 2-chloroacetyl chloride (37.5 mL, 472 mmol). The RM was stirred at 25° C. for 1 h then concentrated in vacuo. The residue was poured into water (300 mL) and the resulting mixture extracted with ethyl acetate (3×300 mL). The combined organic phases were washed with brine (300 mL), dried with anhydrous Na₂SO₄, filtered and concentrated in vacuo to give the title compound as a solid. Y=66%.

### Intermediate B1. 2-Chloro-N-(5-fluoropyrimidin-4-yl) acetamide

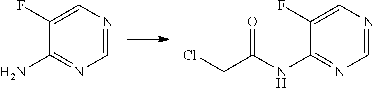 [SMILES: Nc1ncncc1F ; O=C(CCl)Nc1ncncc1F]

To a solution of 5-fluoropyrimidin-4-amine (1.0 g, 8.84 mmol) in CHCl₃ (10 mL) and Et₃N (1.84 mL, 13.2 mmol) at 25° C. under N₂ was added a solution of 2-chloroacetyl chloride (773 μL, 9.72 mmol) in CHCl₃ (4 mL). The mixture was stirred at 25° C. for 4 h. Water (15 mL) was added and the resulting mixture was extracted with DCM (3×15 mL). The combined organic layers were washed with brine (15 mL), dried over Na₂SO₄, filtered, and concentrated under reduced pressure. Purification by FCC (SiO₂, 50% EtOAc in Petroleum ether) gave the title compound as a yellow solid. Y=48%. ¹H NMR (400 MHz, DMSO-d₆) δ 11.14 (s, 1H), 8.90-8.80 (m, 2H), 4.49 (s, 2H).

### Intermediate B2. 2-Chloro-N-(5-fluoropyrimidin-2-yl)acetamide

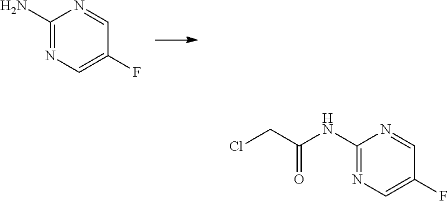 [SMILES: Nc1ncc(F)cn1 ; O=C(CCl)Nc1ncc(F)cn1]

To a solution of 5-fluoropyrimidin-2-amine (2.0 g, 17.7 mmol) in CHCl₃ (20 mL) was added TEA (3.69 mL, 26.5 mmol). The RM was stirred for 30 min at 0° C., then a solution of 2-chloroacetyl chloride (2.11 mL, 26.5 mmol) in CHCl₃ (8 mL) was added. The mixture was stirred at 25° C. for 4 h, diluted with water (20 mL) and extracted with DCM (20 mL×3). The combined organic layers were washed with brine (20 mL), dried over Na₂SO₄, filtered and concentrated under reduced pressure. Prep TLC (SiO₂, 100% EtOAc) gave the title compound as a white solid. Y=54%. ¹H NMR (400 MHz, DMSO-d₆) δ 11.09 (s, 1H), 8.75 (s, 2H), 4.45 (s, 2H).

### Intermediate B3. Chloro-N-pyrimidin-2-yl-acetamide

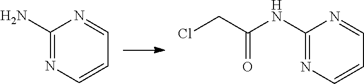 [SMILES: Nc1ncccn1 ; O=C(CCl)Nc1ncccn1]

To a solution of pyrimidin-2-amine (50 g, 526 mmol) in DCM (500 mL) at 0° C. was added TEA (110 mL, 789 mmol) and a solution of 2-chloroacetyl chloride (62.7 mL, 789 mmol) in DCM (50 mL). The RM was stirred at 25° C. for 2 h then diluted with water (500 mL) and DCM (500 mL), filtered, and the solid dried under vacuum to give the title compound as a grey solid. Y=61%. ¹H NMR (400 MHz, DMSO-d₆) δ 10.91 (s, 1H), 8.67 (d, J=5 Hz, 2H), 7.21 (t, J=5 Hz, 1H), 4.52 (s, 2H).

### Intermediate B4. 2-Chloro-N-(5-chloropyrimidin-2-yl)acetamide

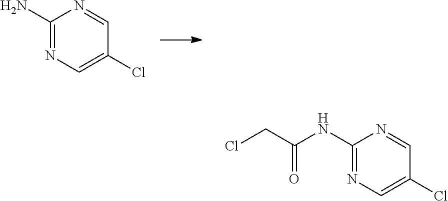 [SMILES: Nc1ncc(Cl)cn1 ; O=C(CCl)Nc1ncc(Cl)cn1]

To a stirred solution of 5-chloropyrimidin-2-amine (10.0 g, 77.2 mmol) in CHCl₃ (100 mL) at 0° C. under N₂ was added Et₃N (16.1 mL, 115 mmol) then a solution of 2-chloroacetyl chloride (116 mmol, 9.2 mL) in CHCl₃ (20 mL) at 0° C. The mixture was stirred at 25° C. for 12 h then diluted with H₂O (150 mL) and the resulting mixture extracted with DCM (3×150 mL). The combined organic layers were washed with brine (150 mL), dried over Na₂SO₄, filtered and concentrated under reduced pressure. The crude material was purified by column chromatography (SiO₂, Petroleum ether/Ethyl acetate=1/1) to give the title compound as a yellow solid, Y=50%. ¹H NMR (400 MHz, DMSO-d₆) δ 11.14 (s, 1H), 8.78 (s, 2H), 4.48 (s, 2H).

### Intermediate B5. 2-Chloro-N-(5-methylpyrimidin-2-yl)acetamide

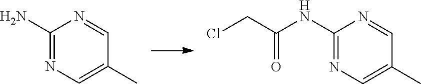

To a solution of 5-methylpyrimidin-2-amine (0.30 g, 2.75 mmol) in CHCl₃ (3 mL) was added Et₃N (572 μL, 4.11 mmol) at 0° C. under N₂, then treated with a solution of 2-chloroacetyl chloride (328 μL, 4.12 mmol) in CHCl₃ (1 mL) at 0° C. The mixture was stirred at 25° C. for 12 h then diluted with H₂O (5 mL) and extracted with DCM (3×5 mL). The combined organic layers were washed with brine (5 mL), dried over Na₂SO₄, filtered and concentrated under reduced pressure. The crude material was purified by column chromatography (SiO₂, Petroleum ether/Ethyl acetate=1/1) to give the title compound as a yellow solid, Y=39%.

### Intermediate B6. 2-Chloro-N-((1s,3s)-3-hydroxy-3-methylcyclobutyl)acetamide

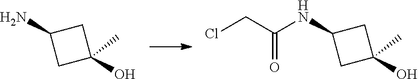

To a solution of cis-3-amino-1-methyl-cyclobutanol (1.4 g, 13.8 mmol) in CHCl₃ (300 mL) was added TEA (2.9 mL, 20.8 mmol) at 0° C., and above mixture was stirred at 0° C. for 20 min, then to the resulting mixture was added a solution of 2-chloroacetyl chloride (1.21 mL, 15.2 mmol) in CHCl₃ (30 mL) at 0° C. The mixture was stirred at 25° C. for 4 h. The reaction mixture was concentrated under reduced pressure. The crude material was purified by prep-TLC (SiO₂, 100% ethyl acetate) to give the title compound as a white solid, Y=45%. ¹H NMR (400 MHz, DMSO-d₆) δ 8.38 (d, J=7 Hz, 1H), 4.98 (s, 1H), 3.98 (s, 2H), 3.85-3.60 (m, 1H), 2.32-2.13 (m, 2H), 1.98-1.82 (m, 2H), 1.21 (s, 3H).

### Intermediate B7. 2-Chloro-N-(oxazol-2-yl)acetamide

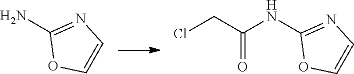 [SMILES: Nc1ncco1 ; O=C(CCl)Nc1ncco1]

To a mixture of oxazol-2-amine (9.5 g, 113 mmol) in DCM (100 mL) was added TEA (226 mmol, 31 mL) and 2-chloroacetyl chloride (9.89 mL, 124 mmol) at 0° C. The mixture was stirred at 25° C. for 2 hours. The reaction mixture was diluted with H₂O (100 mL) and extracted with DCM (100 mL). The organic phase was filtered and the filtrate concentrated under reduced pressure. The crude material was purified by column chromatography (SiO₂, 0 to 100% ethyl acetate in petroleum ether) to give the title compound as a yellow solid, Y=22%. ¹H NMR (400 MHz, DMSO-d₆) δ 11.58 (s, 1H), 7.89 (s, 1H), 7.12 (s, 1H), 4.35 (s, 2H).

### Intermediate C1. 4-Isopropyl-1-methyl-1,6-dihydro-7H-pyrazolo[3,4-d]pyridazin-7-one

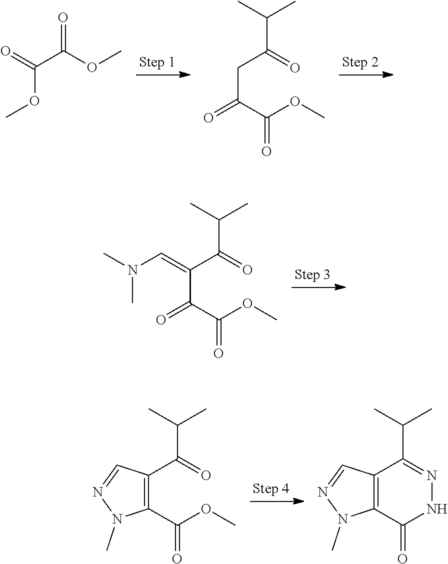

Step 1. Methyl 5-methyl-2,4-dioxo-hexanoate. To a solution of dimethyl oxalate (5.0 g, 42.3 mmol) and 3-methylbutan-2-one (4.98 mL, 46.6 mmol) in toluene (50 mL) was added dropwise a solution of KO^{t}Bu (5.70 g, 50.8 mmol) in THE (20 mL). The mixture was stirred at 25° C. for 2 h. Water (50 mL) was added and the resulting mixture was extracted with ethyl acetate (3×35 mL). The combined organic layers were washed with brine (35 mL), dried over Na₂SO₄, filtered and concentrated under reduced pressure to give the title compound as a white solid. Y=71%.

Step 2. Methyl (3Z)-3-(dimethylaminomethylene)-5-methyl-2,4-dioxo-hexanoate. A mixture of 1,1-dimethoxy-N,N-dimethyl-methanamine (10.4 g, 11.6 mL, 87.1 mmol) and methyl 5-methyl-2,4-dioxo-hexanoate (15 g, 87.1 mmol) was stirred at 25° C. for 6 h. Water (125 mL) was added and the resulting mixture was extracted with DCM (125 mL×3). The combined organic layers were washed with brine (100 mL), dried over Na₂SO₄, filtered, and concentrated under reduced pressure to give the title compound as a yellow liquid. Y=79%.

Step 3. Methyl 2-methyl-4-(2-methylpropanoyl)pyrazole-3-carboxylate. To a solution of methyl (3Z)-3-(dimethylaminomethylene)-5-methyl-2,4-dioxo-hexanoate (3.0 g, 13.2 mmol) in EtOH (80 mL) at 25° C. was added acetic acid (1.3 mL, 22.7 mmol) and a solution of methylhydrazine (40% in water, 3.13 mL) in EtOH (80 mL). The mixture was heated to 60° C. and stirred for 2 h. The reaction mixture was concentrated under reduced pressure to give a residue, which was suspended in water (50 mL) and extracted with EtOAc (3×60 mL). The combined organic layers were washed with brine (60 mL), dried over Na₂SO₄, filtered, and concentrated under reduced pressure to give a residue. Purification by FCC (SiO₂, petroleum ether:EtOAc, 25:1 to 20:1) gave the title compound as a yellow solid. Y=22%. ¹H NMR (400 MHz, DMSO-d₆) δ 8.03 (s, 1H), 3.93 (s, 3H), 3.87 (s, 3H), 3.23-3.16 (m, 1H), 1.06 (d, J=7 Hz, 6H).

Step 4. 4-Isopropyl-1-methyl-6H-pyrazolo[3,4-d]pyridazin-7-one. To a solution of methyl 2-methyl-4-(2-methylpropanoyl)pyrazole-3-carboxylate (300 mg, 1.43 mmol) in EtOH (3 mL) was added hydrazine hydrate (1.42 mL, 28.5 mmol) at 25° C. The mixture was stirred at 80° C. for 2 h. The reaction mixture was filtered, the filter cake was dried under vacuum to give the title compound as a white solid. Y=80%. ¹H NMR (400 MHz, DMSO-d₆) δ 12.60-12.10 (br s, 1H), 8.23 (s, 1H), 4.26 (s, 3H), 3.25-3.13 (m, 1H), 1.27 (d, J=7 Hz, 6H).

### Intermediate C2. 7-Isopropyl-1-methyl-5H-pyrazolo[3,4-d]pyridazin-4-one

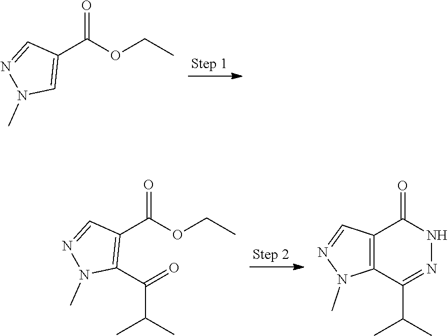 [SMILES: CCOC(=O)c1cnn(C)c1 ; CCOC(=O)c1cnn(C)c1C(=O)C(C)C ; CC(C)c1n[nH]c(=O)c2cnn(C)c12]

Step 1. Ethyl 1-methyl-5-(2-methylpropanoyl) pyrazole-4-carboxylate. To a solution of ethyl 1-methylpyrazole-4-carboxylate (1.0 g, 6.49 mmol) in THE (10 mL) at −78° C. was added a solution of LDA (2M in THF, 4.86 mL, 9.72 mmol) under a N₂ atmosphere. After stirring for 30 mins, a solution of N-methoxy-N,2-dimethyl-propanamide (1.02 g, 7.78 mmol) in THE (10 mL) was added at −78° C. The resulting mixture was stirred at 25° C. for 30 min under N₂. Saturated NH₄Cl aqueous solution (5 mL) and water (10 mL) were added and the resulting mixture was extracted with EtOAc (3×10 mL). The combined organic layers were washed with brine (3×10 mL), dried over Na₂SO₄, filtered, and concentrated under reduced pressure. Purification by FCC (SiO₂, 10-25% EtOAc in petroleum ether) gave the title compound as a colourless oil. Y=25%. ¹H NMR (400 MHz, DMSO-d₆) δ 7.90 (s, 1H), 4.22 (q, J=7 Hz, 2H), 3.77 (s, 3H), 3.32-3.27 (m, 1H), 1.25 (t, J=7 Hz, 3H), 1.09 (d, J=7 Hz, 6H).

Step 2. 7-Isopropyl-1-methyl-5H-pyrazolo[3,4-d]pyridazin-4-one. To a solution of ethyl 1-methyl-5-(2-methylpropanoyl)pyrazole-4-carboxylate (150 mg, 669 μmol) in EtOH (1 mL) was added hydrazine hydrate (331 μL, 6.69 mmol) at 25° C. The mixture was stirred at 80° C. for 12 h. The reaction mixture was filtered and dried under reduced pressure to give the title compound as a white solid. Y=86%. ¹H NMR (400 MHz, DMSO-d₆) δ 12.4-12.3 (br. s, 1H), 8.17 (s, 1H), 4.23 (s, 3H), 3.60-3.53 (m, 1H), 1.28 (d, J=6 Hz, 6H).

### Intermediate C3. 7-Isopropyl-2-methyl-5H-pyrazolo[3,4-d]pyridazin-4-one

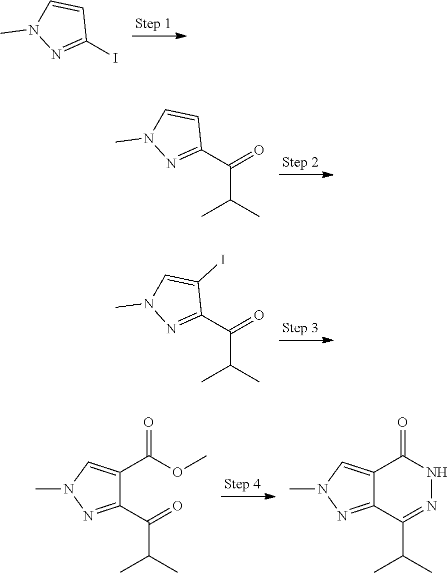

Step 1. 2-Methyl-1-(1-methylpyrazol-3-yl)propan-1-one. To a solution of 1M isopropylmagnesium chloride (6.25 mL, 6.25 mmol) in THE (13 mL) at 0° C. was added a solution of 3-iodo-1-methyl-pyrazole (1.3 g, 6.25 mmol) in THE (5 mL). After stirring for 1 h at 25° C., a solution of N-methoxy-N,2-dimethyl-propanamide (574 mg, 4.38 mmol) in THE (3 mL) was added and the RM stirred at 25° C. for 12 h. Saturated NH₄Cl aqueous solution (5 mL) was added and the resulting mixture was extracted with EtOAc (3×10 mL). The combined organic layers were washed with brine (3×10 mL), dried over Na₂SO₄, filtered, and concentrated under reduced pressure to give a residue. Purification by preparative TLC (SiO₂, 25% EtOAc in petroleum ether) gave the title compound as a red solid. Y=63%. ¹H NMR (400 MHz, DMSO-d₆) δ 7.82 (d, J=2 Hz, 1H), 6.69 (d, J=2 Hz, 1H), 3.94 (s, 3H), 3.66-3.56 (m, 1H), 1.09 (d, J=7 Hz, 6H).

Step 2. 1-(4-Iodo-1-methyl-pyrazol-3-yl)-2-methyl-propan-1-one. To a solution of 2-methyl-1-(1-methylpyrazol-3-yl)propan-1-one (550 mg, 3.61 mmol) in AcOH (5.7 mL) at 25° C. was added N-iodosuccinimide (1.22 g, 5.42 mmol). The mixture was stirred at 25° C. for 12 h, diluted with water (6 mL) and extracted with EtOAc (3×6 mL). The combined organic layers were washed with brine (3×6 mL), dried over Na₂SO₄, filtered, and concentrated under reduced pressure. Purification by FCC (SiO₂, 10-25% EtOAc in petroleum ether) gave the title compound as a red solid. Y=85%. ¹H NMR (400 MHz, DMSO-d₆) δ 8.04 (s, 1H), 3.93 (s, 3H), 3.70-3.60 (m, 1H), 1.07 (d, J=7 Hz, 6H).

Step 3. Methyl 1-methyl-3-(2-methylpropanoyl)pyrazole-4-carboxylate. To a solution of 1-(4-iodo-1-methyl-pyrazol-3-yl)-2-methyl-propan-1-one (0.40 g, 1.44 mmol) in MeOH (12.8 mL) at 25° C. was added TEA (1.0 mL, 7.19 mmol) and PdCl₂(dppf) (105 mg, 144 μmol). The suspension was degassed and purged with CO (gas) three times. The mixture was stirred under CO (gas, 50 Psi) at 70° C. for 12 h. The reaction mixture was filtered through a pad of Celite. The filtrate was diluted with water (5 mL) and extracted with EtOAc (3×5 mL). The combined organic layers were washed with brine (3×5 mL), dried over Na₂SO₄, filtered. and concentrated under reduced pressure. Purification by FCC (SiO₂, 10-50% EtOAc in petroleum ether) gave the title compound as a yellow oil. ¹H NMR (400 MHz, DMSO-d₆) δ 8.34 (s, 1H), 3.91 (s, 3H), 3.71 (s, 3H), 3.44-3.36 (m, 1H), 1.07 (d, J=7 Hz, 6H).

Step 4. 7-Isopropyl-2-methyl-5H-pyrazolo[3,4-d]pyridazin-4-one. To a solution of methyl 1-methyl-3-(2-methylpropanoyl)pyrazole-4-carboxylate (150 mg, 714 μmol) in EtOH (1.5 mL) was added hydrazine hydrate (354 μL, 7.14 mmol) at 25° C. The resulting mixture was stirred at 80° C. for 12 h. The RM was concentrated under reduced pressure to give the title compound as a white solid. Y=88%. ¹H NMR (400 MHz, DMSO-d₆) δ 11.93 (br. s, 1H), 8.62 (s, 1H), 4.12 (s, 3H), 3.27-3.24 (m, 1H), 1.29 (d, J=7 Hz, 6H).

### Intermediate C4. 2-(Cyclopropylmethyl)-7-isopropyl-5H-pyrazolo[3,4-d]pyridazin-4-one

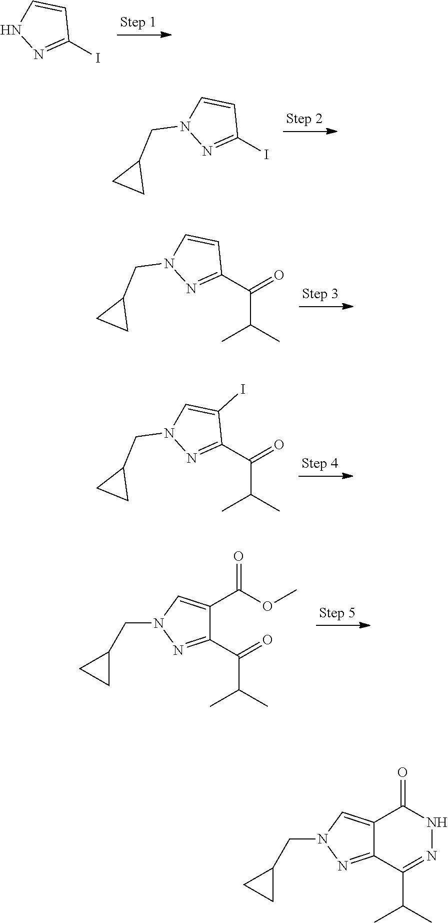 [SMILES: Ic1cc[nH]n1 ; Ic1ccn(CC2CC2)n1 ; CC(C)C(=O)c1ccn(CC2CC2)n1 ; CC(C)C(=O)c1nn(CC2CC2)cc1I ; COC(=O)c1cn(CC2CC2)nc1C(=O)C(C)C ; CC(C)c1n[nH]c(=O)c2cn(CC3CC3)nc12]

Step 1. 1-(Cyclopropylmethyl)-3-iodo-pyrazole. To a solution of 3-iodo-1H-pyrazole (4.0 g, 20.6 mmol) in DMF (40 mL) at 25° C. was added bromomethylcyclopropane (1.97 mL, 20.6 mmol) and K₂CO₃ (8.55 g, 61.9 mmol). The RM was stirred for 12 h, diluted with water (40 mL) and extracted with EtOAc (3×40 mL). The combined organic layers were washed with brine (3×40 mL), dried over Na₂SO₄, filtered, and concentrated under reduced pressure. Purification by FCC (SiO₂, 10-20% EtOAc in petroleum ether) gave the title compound as a yellow oil. Y=92%. ¹H NMR (400 MHz, DMSO-d₆) δ 7.68 (d, J=2 Hz, 1H), 6.44 (d, J=2 Hz, 1H), 3.96 (d, J=7 Hz, 2H), 1.23-1.19 (m, 1H), 0.50-0.47 (m, 2H), 0.35-0.33 (m, 2H).

Step 2. 1-[1-(Cyclopropylmethyl)pyrazol-3-yl]-2-methyl-propan-1-one. To a solution of 1M isopropylmagnesium chloride lithium chloride (8.06 mL, 8.06 mmol) in THE (20 mL) was added a solution of 1-(cyclopropylmethyl)-3-iodo-pyrazole (2.0 g, 8.06 mmol) in THE (8 mL) at 0° C. The mixture was stirred at 25° C. for 1 h, then a solution of N-methoxy-N-(2-dimethyl)-propanamide (740 mg, 5.64 mmol) in THE (3 mL) was added at 25° C. The RM was stirred at 25° C. for 12 h. The RM was quenched with saturated NH₄Cl aqueous solution (10 mL), diluted with water (20 mL) and extracted with EtOAc (3×20 mL). The combined organic layers were washed with brine (3×20 mL), dried over Na₂SO₄, filtered, and concentrated under reduced pressure. Purification by FCC (SiO₂, 10-25% EtOAc in petroleum ether) gave the title compound as a yellow oil. Y=65%. ¹H NMR (400 MHz, DMSO-d₆) δ 7.90 (d, J=2 Hz, 1H), 6.71 (d, J=2 Hz, 1H), 4.07 (d, J=7 Hz, 2H), 3.65-3.58 (m, 1H), 1.29-1.25 (m, 1H), 1.10 (d, J=7 Hz, 6H), 0.56-0.52 (m, 2H), 0.41-0.38 (m, 2H).

Step 3. 1-[1-(Cyclopropylmethyl)-4-iodo-pyrazol-3-yl]-2-methyl-propan-1-one. To a solution of 1-[1-(cyclopropylmethyl)pyrazol-3-yl]-2-methyl-propan-1-one (500 mg, 2.60 mmol) in AcOH (5 mL) at 25° C. was added N-iodosuccinimide (878 mg, 3.90 mmol). The RM was stirred for 12 h. The reaction mixture was diluted with water (6 mL) and extracted with EtOAc (3×6 mL). The combined organic layers were washed with brine (3×6 mL), dried over Na₂SO₄, filtered, and concentrated under reduced pressure. Purification by FCC (SiO₂, 10-30% EtOAc in petroleum ether) gave the title compound as a yellow solid. Y=60%. ¹H NMR (400 MHz, DMSO-d₆) δ 8.12 (s, 1H), 4.06 (d, J=7 Hz, 2H), 3.68-3.61 (m, 1H), 1.30-1.24 (m, 1H), 1.08 (d, J=7 Hz, 6H), 0.56-0.52 (m, 2H), 0.41-0.38 (m, 2H).

Step 4. Methyl 1-(cyclopropylmethyl)-3-(2-methylpropanoyl)pyrazole-4-carboxylate. To a solution of 1-[1-(cyclopropylmethyl)-4-iodo-pyrazol-3-yl]-2-methyl-propan-1-one (420 mg, 1.32 mmol) in MeOH (5 mL) was added TEA (919 μL, 6.60 mmol) and PdCl₂(dppf) (96 mg, 132 μmol) at 25° C. The suspension was degassed and purged with CO (gas) three times. The mixture was stirred under CO (gas, 50 Psi) at 70° C. for 12 h. The reaction mixture was filtered through a pad of Celite. The filtrate was diluted with water (5 mL) and extracted with EtOAc (3×5 mL). The combined organic layers were washed with brine (3×5 mL), dried over Na₂SO₄, filtered and concentrated under reduced pressure. Purification by FCC (SiO₂, 10-50% EtOAc in petroleum ether) gave the title compound as a red oil. Y=88%. ¹H NMR (400 MHz, DMSO-d₆) δ 8.41 (s, 1H), 4.05 (d, J=7 Hz, 2H), 3.72 (s, 3H), 3.45-3.37 (m, 1H), 1.33-1.24 (m, 1H), 1.08 (d, J=7 Hz, 6H), 0.56-0.56 (m, 2H), 0.40-0.39 (m, 2H).

Step 5. 2-(Cyclopropylmethyl)-7-isopropyl-5H-pyrazolo[3,4-d]pyridazin-4-one. To a solution of methyl 1-(cyclopropylmethyl)-3-(2-methylpropanoyl)pyrazole-4-carboxylate (290 mg, 1.16 mmol) in EtOH (1.5 mL) was added hydrazine hydrate (575 μL, 11.6 mmol) at 25° C. The resulting mixture was stirred at 80° C. for 12 h. The reaction mixture was filtered and dried in vacuo to give the title compound as a white solid. Y=74%. ¹H NMR (400 MHz, DMSO-d₆) δ 11.91 (s, 1H), 8.70 (s, 1H), 4.25 (d, J=7 Hz, 2H), 3.31-3.25 (m, 1H), 1.41-1.34 (m, 1H), 1.30 (d, J=7 Hz, 6H), 0.60-0.53 (m, 2H), 0.48-0.41 (m, 2H).

### Intermediate C5. 2-Ethyl-7-isopropyl-5H-pyrazolo[3,4-d]pyridazin-4-one

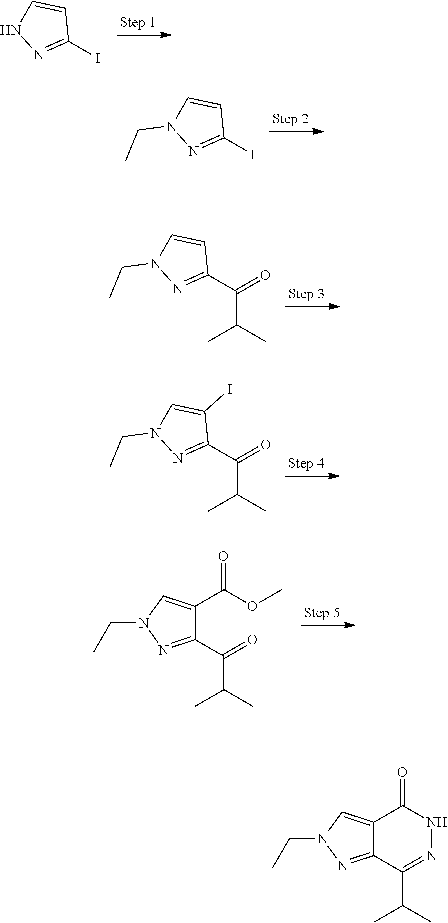

Step 1. 1-Ethyl-3-iodo-pyrazole. To a solution of 3-iodo-1H-pyrazole (6.0 g, 30.9 mmol) in DMF (60 mL) was added Cs₂CO₃ (30.2 g, 92.8 mmol). The mixture was stirred at 25° C. for 10 min, then iodoethane (4.95 mL, 61.9 mmol) was added. The RM was stirred for 2 h, diluted with water (60 mL) and extracted with EtOAc (3×60 mL). The combined organic layers were washed with brine (3×60 mL), dried over Na₂SO₄, filtered, and concentrated under reduced pressure. Purification by FCC (SiO₂, 10-20% EtOAc in petroleum ether) gave the title compound as a colourless oil. Y=41%. ¹H NMR (400 MHz, DMSO-d₆) δ 7.66 (d, J=2 Hz, 1H), 6.43 (d, J=2 Hz, 1H), 4.13 (q, J=7 Hz, 2H), 1.34 (t, J=7 Hz, 3H).

Step 2. 1-(1-Ethylpyrazol-3-yl)-2-methyl-propan-1-one. To a solution of 1M isopropyl magnesium chloride lithium chloride (8.33 mL, 8.33 mmol) in THE (20 mL) at 0° C. was added a solution of 1-ethyl-3-iodo-pyrazole (1.85 g, 8.33 mmol) in THE (7 mL), and the resulting mixture was stirred at 0° C. for 1 h. A solution of N-methoxy-N-(2-dimethyl)propanamide (765 mg, 5.83 mmol) in THE (10 mL) was added. The RM was stirred at 25° C. for 12 h under N₂, then quenched with aqueous saturated NH₄Cl (5 mL) and water (20 mL). The mixture was extracted with EtOAc (3×20 mL) and the combined organic layers were washed with brine (3×20 mL), dried over Na₂SO₄, filtered and concentrated under reduced pressure. Purification by FCC (SiO₂, 10-16% EtOAc in petroleum ether) gave the title compound as a red solid. Y=72%. ¹H NMR (400 MHz, DMSO-d₆) δ 7.87 (d, J=2 Hz, 1H), 6.70 (d, J=2 Hz, 1H), 4.23 (q, J=7 Hz, 2H), 3.66-3.56 (m, 1H), 1.40 (t, J=7 Hz, 3H), 1.09 (d, J=7 Hz, 6H).

Step 3. 1-(1-Ethyl-4-iodo-pyrazol-3-yl)-2-methyl-propan-1-one. To a solution of 1-(1-ethylpyrazol-3-yl)-2-methyl-propan-1-one (900 mg, 5.41 mmol) in AcOH (9 mL) at 25° C. was added N-iodosuccinimide (1.83 g, 8.12 mmol). The RM was stirred at 25° C. for 12 h, diluted with water (10 mL) and extracted with EtOAc (3×10 mL). The combined organic layers were washed with brine (3×10 mL), dried over Na₂SO₄, filtered, and concentrated under reduced pressure. Purification by FCC (SiO₂, 10-25% EtOAc in petroleum ether) gave the title compound as a yellow oil. Y=89%. ¹H NMR (400 MHz, DMSO-d₆) δ 8.11 (s, 1H), 4.22 (q, J=7 Hz, 2H), 3.68-3.61 (m, 1H), 1.40 (t, J=7 Hz, 3H), 1.07 (d, J=7 Hz, 6H).

Step 4. Methyl 1-ethyl-3-(2-methylpropanoyl)pyrazole-4-carboxylate. To a solution of 1-(1-ethyl-4-iodo-pyrazol-3-yl)-2-methyl-propan-1-one (1.4 g, 4.79 mmol) in MeOH (15 mL) at 25° C. was added TEA (3.34 mL, 24.0 mmol) and PdCl₂(dppf) (351 mg, 479 μmol). The suspension was degassed and purged with CO (gas) 3 times. The mixture was stirred under CO (gas, 50 psi) at 70° C. for 12 h. The reaction mixture was concentrated under reduced pressure. The residue was filtered through a pad of Celite, diluted with water (15 mL) and extracted with EtOAc (3×15 mL). The combined organic layers were washed with brine (3×15 mL), dried over Na₂SO₄, filtered, and concentrated under reduced pressure. Purification by FCC (SiO₂, 10-50% EtOAc in petroleum ether) gave the title compound as a red oil. Y=93%. ¹H NMR (400 MHz, DMSO-d₆) δ 8.40 (s, 1H), 4.21 (q, J=7 Hz, 2H), 3.71 (s, 3H), 3.42-3.36 (m, 1H), 1.40 (t, J=7 Hz, 3H), 1.08 (d, J=7 Hz, 6H).

Step 5. 2-Ethyl-7-isopropyl-5H-pyrazolo[3,4-d]pyridazin-4-one. To a solution of methyl 1-ethyl-3-(2-methylpropanoyl)pyrazole-4-carboxylate (1.0 g, 4.46 mmol) in EtOH (5 mL) at 25° C. was added hydrazine hydrate (2.28 g, 44.6 mmol). The resulting mixture was stirred at 80° C. for 12 h. The reaction mixture was filtered and the solid dried under reduced pressure to give the title compound as a white solid. Y=87%. ¹H NMR (400 MHz, DMSO-d₆) δ 11.90 (s, 1H), 8.69 (s, 1H), 4.41 (q, J=7 Hz, 2H), 3.30-3.25 (m, 1H), 1.47 (t, J=7 Hz, 3H), 1.30 (d, J=7 Hz, 6H)

### Intermediate C6. 2-(2-Ethyl-7-isopropyl-4-oxo-pyrazolo[3,4-d]pyridazin-5-yl)acetic acid

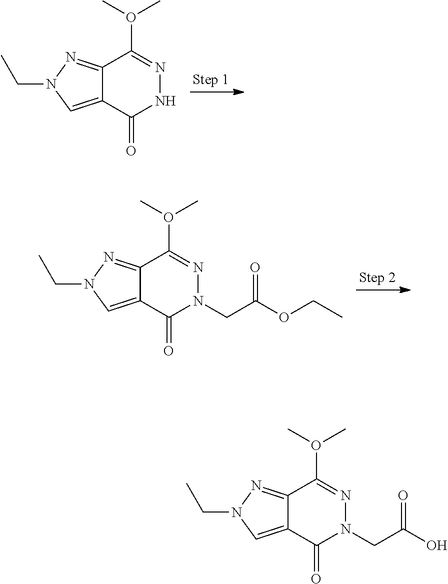

Step 1. Ethyl 2-(2-ethyl-7-isopropyl-4-oxo-pyrazolo[3,4-d]pyridazin-5-yl)acetate. To a solution of ethyl 2-bromoacetate (121 μL, 1.09 mmol) in DMF (2 mL) at 25° C. was added K₂CO₃ (302 mg, 2.18 mmol) and 2-ethyl-7-isopropyl-5H-pyrazolo[3,4-d]pyridazin-4-one (150 mg, 727 μmol). The RM was stirred at 80° C. for 5 h, diluted with water (3 mL) and extracted with EtOAc (3×3 mL). The combined organic layers were washed with brine (3×3 mL), dried over Na₂SO₄, filtered, and concentrated under reduced pressure. Purification by FCC (SiO₂, 10-100% EtOAc in petroleum ether) gave the title compound as a yellow oil. Y=80%. ¹H NMR (400 MHz, DMSO-d₆) δ 8.77 (s, 1H), 4.79 (s, 2H), 4.43 (q, J=7 Hz, 2H), 4.13 (q, J=7 Hz, 2H), 3.30-3.27 (m, 1H), 1.49 (t, J=7 Hz, 3H), 1.31 (d, J=7 Hz, 6H), 1.19 (t, J=7 Hz, 3H).

Step 2. 2-(2-Ethyl-7-isopropyl-4-oxo-pyrazolo[3,4-d]pyridazin-5-yl)acetic acid. To a solution of ethyl 2-(2-ethyl-7-isopropyl-4-oxo-pyrazolo[3,4-d]pyridazin-5-yl)acetate (170 mg, 582 μmol) in THE (2 mL) and water (0.5 mL) at 25° C. was added LiOH·H₂O (48.8 mg, 1.16 mmol). The RM was stirred at 25° C. for 3 h, diluted with water (3 mL) and extracted with EtOAc (3×3 mL). 2M HCl was added dropwise until pH=4-5. The resulting mixture was extracted with EtOAc (3×1 mL). The combined organic layers were washed with brine (3×1 mL), dried over Na₂SO₄, filtered, and concentrated under reduced pressure to give the title compound as a white solid. Y=78%. ¹H NMR (400 MHz, DMSO-d₆) δ 12.96-12.89 (m, 1H), 8.76 (s, 1H), 4.68 (s, 2H), 4.43 (q, J=7 Hz, 2H), 3.30-3.26 (m, 1H), 1.48 (t, J=7 Hz, 3H), 1.31 (d, J=7 Hz, 6H).

### Intermediate C7. 4-Isopropyl-2-methyl-2,6-dihydro-7H-pyrazolo[3,4-d]pyridazin-7-one

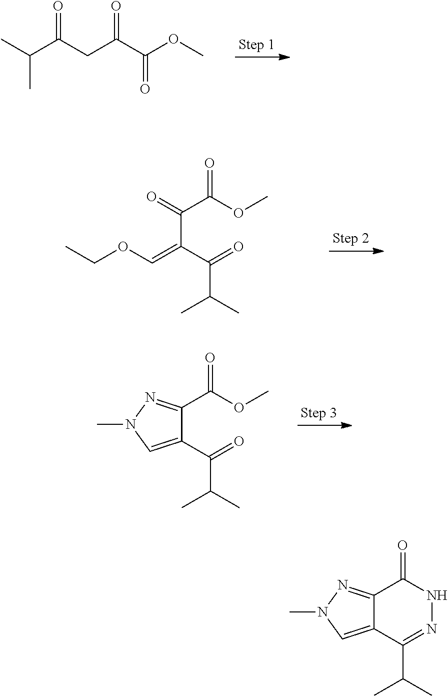

Step 1. Methyl-3-(ethoxymethylene)-5-methyl-2,4-dioxohexanoate. To a solution of methyl 5-methyl-2,4-dioxo-hexanoate (2.0 g, 11.6 mmol) in Ac₂O (2.18 mL, 23.2 mmol) at 25° C. was added diethoxymethoxyethane (2.32 mL, 13.9 mmol). The RM was stirred at 120° C. for 4 h under N₂ then concentrated in vacuo to give the title compound as a brown oil. Y=quantitative.

Step 2. Methyl 4-isobutyryl-1-methyl-1H-pyrazole-3-carboxylate. To a solution of methyl-3-(ethoxymethylene)-5-methyl-2,4-dioxohexanoate (2.6 g, 11.4 mmol) in THE (30 mL) at 0° C. was added methyl hydrazine (1.50 mL, 40% in THF, 11.4 mmol). The RM was stirred at 0° C. for 4 h under N₂ then concentrated in vacuo. Purification by prep-HPLC (column: Agela DuraShell C18, 250×70 mm, 10 μm; mobile phase: [water (10 mM NH₄HCO₃)−ACN]; B: 12-42%, 20 min) gave the title compound as a white solid. Y=40%. ¹H NMR (400 MHz, DMSO-d₆) δ 8.44 (s, 1H), 3.90 (s, 3H), 3.79 (s, 3H), 3.23-3.14 (m, 1H), 1.05 (d, J=7 Hz, 6H).

Step 3. 4-Isopropyl-2-methyl-6H-pyrazolo [3,4-d]pyridazin-7-one. To a solution of methyl 4-isobutyryl-1-methyl-1H-pyrazole-3-carboxylate (300 mg, 1.43 mmol) in EtOH (3 mL) at 25° C. was added hydrazine hydrate (1.42 mL, 28.5 mmol). The RM was stirred at 80° C. for 4 h under N₂ then concentrated under vacuum to give the title compound as a brown oil. Y=quantitative.

### Intermediate C8. 2-Chloro-7-isopropyl-5H-thieno[2,3-d]pyridazin-4-one

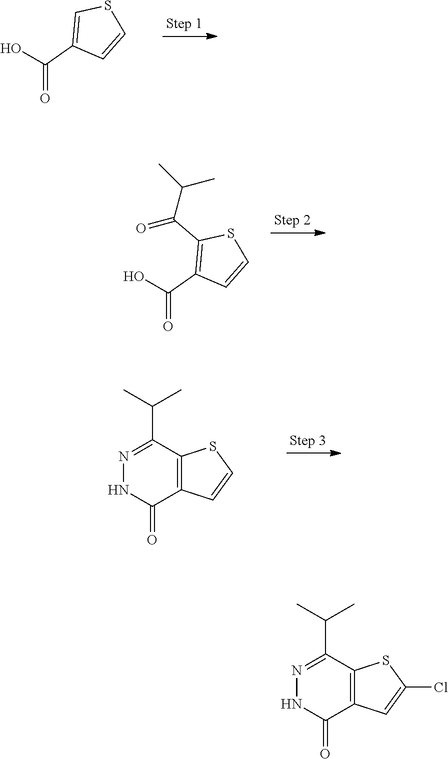

Step 1. 2-(2-Methylpropanoyl)thiophene-3-carboxylic acid. To a solution of thiophene-3-carboxylic acid (1.0 g, 7.80 mmol) in THE (10 mL) at 0° C. was added 2 M LDA in THE (8.59 mL, 17.18 mmol) dropwise with stirring under N₂. The resulting mixture was stirred for 15 min. A solution of N-methoxy-N,2-dimethylpropanamide (1.13 g, 8.58 mmol) in THE (5 mL) was added dropwise at 0° C. The RM was stirred at 25° C. for 2 h. The mixture was adjusted to pH 4 with 2 M HCl. The resulting mixture was extracted with EtOAc (3×10 mL). The combined organic phase was washed with brine (5 mL), dried with anhydrous Na₂SO₄, filtered, and concentrated under vacuum to give the title compound as a red solid. ¹H NMR (400 MHz, DMSO-d₆) δ 13.14 (br. s, 1H), 7.84 (d, J=5 Hz, 1H), 7.37 (d, J=5 Hz, 1H), 3.35-3.25 (m, 1H), 1.10 (d, J=7 Hz, 6H).

Step 2. 7-Isopropyl-5H-thieno[2,3-d]pyridazin-4-one. To a solution of 2-(2-methylpropanoyl)thiophene-3-carboxylic acid (650 mg, 3.28 mmol) in EtOH (8 mL) was added hydrazine hydrate (1.63 mL, 32.8 mmol) and the RM was stirred at 80° C. for 1 h. The mixture was concentrated under reduced pressure to give the title compound as a white solid. Y=71%.

Step 3. 2-Chloro-7-isopropyl-5H-thieno[2,3-d]pyridazin-4-one. To a solution of 7-isopropyl-5H-thieno[2,3-d]pyridazin-4-one (90 mg, 463 μmol) in DMF (1 mL) at 25° C. was added N-chlorosuccinimide (124 mg, 927 μmol). The RM was stirred at 25° C. for 1 h. Saturated aqueous Na₂S₂O₃ solution (2 mL) was added and the mixture was stirred at 0° C. for 10 min. The mixture was extracted with ethyl acetate (3×2 mL). The combined organic phase was washed with brine (2 mL), dried over anhydrous Na₂SO₄, filtered, and concentrated in vacuo. Purification by prep-TLC (SiO₂, 100% Ethyl acetate) gave the title compound as a white solid. Y=66%.

### Intermediate C9. 2-Chloro-4-isopropyl-6H-thieno[2,3-d]pyridazin-7-one

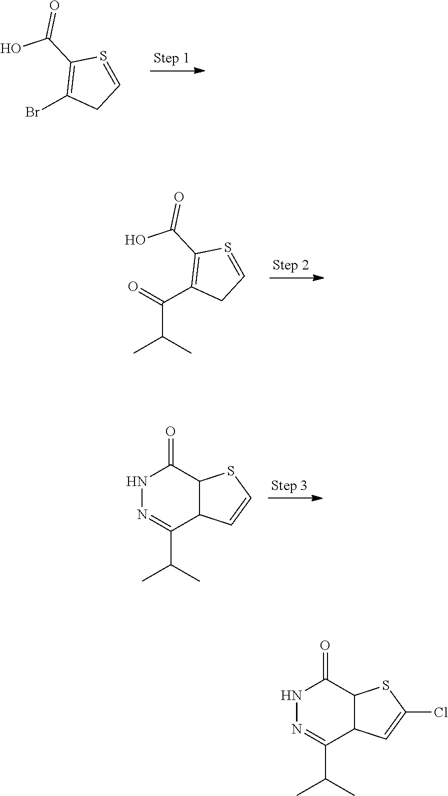

Step 1. 3-(2-Methylpropanoyl)thiophene-2-carboxylic acid. To a solution of 3-bromothiophene-2-carboxylic acid (1.6 g, 7.73 mmol) in THE (16 mL) at −78° C. was added n-BuLi (2.5 M, 6.18 mL, 15.5 mmol) and the resulting mixture was stirred for 0.5 h. N-methoxy-N,2-dimethyl-propanamide (1.22 g, 9.27 mmol) was added under N₂ at −78° C. and the RM was stirred at 25° C. for 1 h. The reaction mixture was quenched by addition of saturated aqueous NH₄Cl (10 mL) at 0° C., then diluted with 1 M HCl (8 mL). The mixture was extracted with EtOAc (3×20 mL). The combined organic layers were washed with brine (15 mL), dried over Na₂SO₄, filtered and concentrated under reduced pressure. Purification by FCC (SiO₂, 50-100% EtOAc in petroleum ether) gave the title compound as a yellow solid. Y=78%.

Step 2. 4-Isopropyl-6H-thieno[2,3-d]pyridazin-7-one. To a solution of 3-(2-methylpropanoyl)thiophene-2-carboxylic acid (500 mg, 2.52 mmol) in EtOH (6 mL) at 25° C. was added hydrazine hydrate (2.58 g, 50.4 mmol). The solution was stirred at 80° C. for 12 h. The reaction was filtered and the filter cake dried in vacuo to give the title compound as a yellow solid. Y=80%. ¹H NMR (400 MHz, DMSO-d₆) δ 12.69 (s, 1H), 8.24 (d, J=6 Hz, 1H), 7.70 (d, J=6 Hz, 1H), 3.42-3.34 (m, 1H), 1.27 (d, J=7 Hz, 6H).

Step 3. 2-Chloro-4-isopropyl-6H-thieno[2,3-d]pyridazin-7-one. To a solution of 4-isopropyl-6H-thieno[2,3-d]pyridazin-7-one (180 mg, 927 μmol) in THE (3 mL) at −78° C. was added LDA (2 M in THF, 1.16 mL, 2.32 mmol). After 30 min, hexachloroethane (105 μL, 927 μmol) was added at −25° C. The RM was stirred at 0° C. for 2 h. The reaction mixture was quenched by addition of water (2 mL) at 0° C. and extracted with EtOAc (3×2 mL). The combined organic layers were washed with brine (2×2 mL), dried over Na₂SO₄, filtered, and concentrated under reduced pressure. Purification by FCC (20-50% EtOAc in petroleum ether) gave the title compound as a white solid. Y=71%.

### Intermediate C10. 2-Ethyl-7-isopropyl-5H-thieno[2, 3-d]pyridazin-4-one

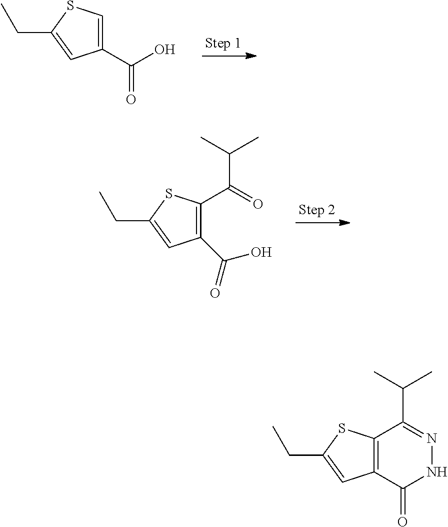

Step 1. 5-Ethyl-2-(2-methylpropanoyl) thiophene-3-carboxylic acid. To a solution of 5-ethylthiophene-3-carboxylic acid (500 mg, 3.20 mmol) in THE (6 mL) at −78° C. was added LDA (2 M in THF, 3.20 mL, 6.40 mmol) under N₂ and the mixture was stirred for 1 h. N-methoxy-N,2-dimethyl propanamide (420 mg, 3.20 mmol) was added. After stirring for 2 h, water (5 mL) was added and the resulting mixture was extracted with ethyl acetate (3×5 mL). The combined organic layers were washed with brine (5 mL), dried over anhydrous Na₂SO₄, filtered and concentrated under reduced pressure. FCC (SiO₂, 50% EtOAc in Petroleum ether) gave the title compound as a white solid. Y=73%. ¹H NMR (400 MHz, DMSO-d₆) δ 13.23 (br. s, 1H), 7.14 (s, 1H), 3.30-3.23 (m, 1H), 2.87-2.80 (m, 2H), 1.27-1.23 (m, 3H), 1.09 (d, J=7 Hz, 6H).

Step 2. 2-Ethyl-7-isopropyl-5H-thieno[2, 3-d]pyridazin-4-one. To a solution of 5-ethyl-2-(2-methylpropanoyl)thiophene-3-carboxylic acid (230 mg, 1.02 mmol) in EtOH (2 mL) at 25° C. was added hydrazine hydrate (1.04 g, 20.3 mmol) and the mixture was stirred at 80° C. for 2 h. The solution was concentrated under vacuum. The residue was triturated with petroleum ether at 25° C. for 5 min, then the solid was collected by filtration and dried in vacuo to give the title compound as a white solid. Y=31%.

### Intermediate C11. 1-Methyl-7-(prop-1-en-2-yl)-1H,4H,5H-imidazo[4,5-d]pyridazine-4-one

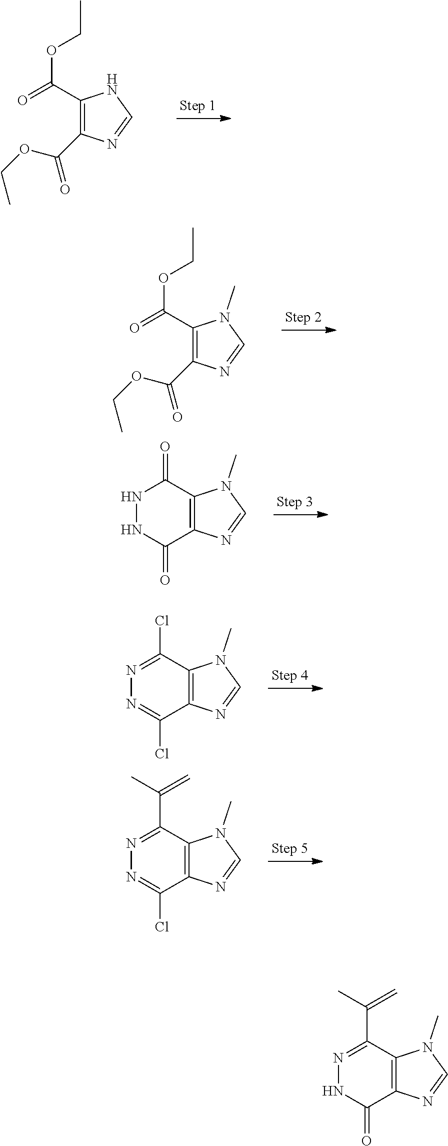

Step 1. Diethyl 1-methylimidazole-4,5-dicarboxylate. To a solution of diethyl 1H-imidazole-4,5-dicarboxylate (10 g, 47.1 mmol) in DMF (150 mL) at 25° C. was added K₂CO₃ (13.0 g, 94.3 mmol) and CH₃I (2.93 mL, 47.1 mmol). The RM was stirred for 2 h. Water (50 mL) was added and the resulting mixture was extracted with EtOAc (3×50 mL). The combined organic phase was washed with brine (50 mL), dried with anhydrous Na₂SO₄, filtered and concentrated under vacuum to give the title compound as a brown oil. ¹H NMR (400 MHz, DMSO-d₆) δ 7.89 (s, 1H), 4.38-4.12 (m, 4H), 3.77 (s, 3H), 1.35-1.25 (m, 6H).

Step 2. 3-Methyl-5,6-dihydroimidazo[4,5-d]pyridazine-4,7-dione. To a solution of diethyl 1-methylimidazole-4,5-dicarboxylate (10 g, 44.2 mmol) in EtOH (250 mL) at 25° C. was added hydrazine hydrate (13.2 mL, 265 mmol). The RM was stirred at 80° C. for 2 h then cooled and the resulting solid collected by filtration to give the title compound as a white solid.

Step 3. 4,7-Dichloro-3-methyl-imidazo[4,5-d]pyridazine. A mixture of 3-methyl-5,6-dihydroimidazo[4,5-d]pyridazine-4,7-dione (600 mg 3.61 mmol) and POCl₃ (10 mL) was stirred at 100° C. for 12 h. The solution was concentrated under vacuum and azeotroped with chloroform (3×20 mL) to give the title compound as a yellow gum.

Step 4. 4-Chloro-7-isopropenyl-1-methyl-imidazo[4,5-d]pyridazine. To a solution of 4,7-dichloro-3-methyl-imidazo[4,5-d]pyridazine (700 mg, 3.45 mmol) in water (3 mL) and THE (15 mL) at 25° C. was added 2-isopropenyl-4,4,5,5-tetramethyl-1,3,2-dioxaborolane (579 mg, 3.45 mmol), Na₂CO₃ (1.83 g, 17.2 mmol) and Pd(PPh₃)₄ (398 mg, 345 μmol). The RM was stirred at 90° C. for 12 h under N₂, then diluted with water (10 mL) and extracted with ethyl acetate (3×10 mL). The combined organic phase was washed with brine (10 mL), dried with anhydrous Na₂SO₄, filtered, and concentrated in vacuum. Purification by prep-HPLC (column: Kromasil C18 (250×50 mm, 10 μm); mobile phase: [water (10 mM NH₄HCO₃)−ACN]; B: 1-20%, 10 min) gave the title compound as a white solid. Y=9% ¹H NMR (400 MHz, DMSO-d₆) δ 8.71 (s, 1H), 5.71 (s, 1H), 5.29 (s, 1H), 3.90 (s, 3H), 2.28 (s, 3H).

Step 5. 1-Methyl-7-(prop-1-en-2-yl)-1H,4H,5H-imidazo[4,5-d]pyridazine-4-one. To a solution of 4-chloro-7-isopropenyl-1-methyl-imidazo[4,5-d]pyridazine (65 mg, 312 μmol) in AcOH (2 mL) at 25° C. was added NaOAc (76.7 mg, 934 μmol). The RM was stirred at 50° C. for 12 h, then concentrated in vacuo to give the title compound as a white solid (y=quantitative).

### Intermediate C12. 2,7-Diisopropyl-2,5-dihydro-4H-pyrazolo[3,4-d]pyridazin-4-one

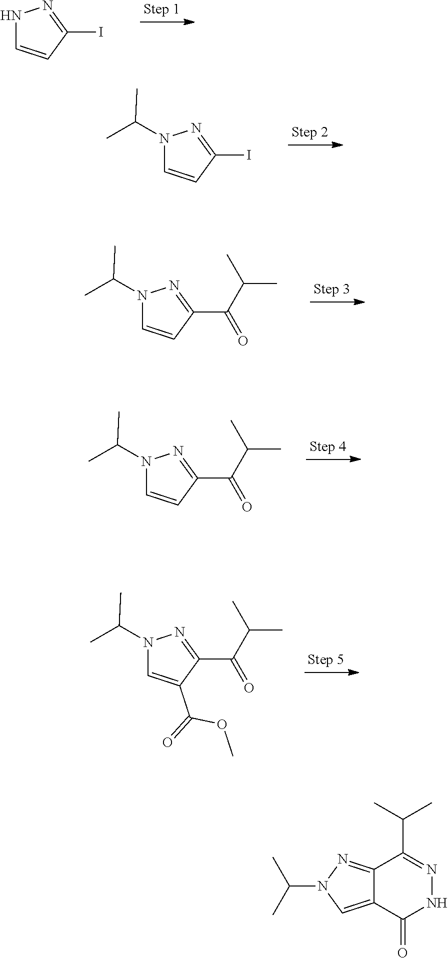

Step 1. 3-Iodo-1-isopropyl-pyrazole. To a solution of 3-iodo-1H-pyrazole (5.0 g, 25.8 mmol) in DMF (1 mL) at 0° C. was added NaHMDS (1 M in THF, 30.9 mL, 30.9 mmol). After stirring for 30 mins, 2-iodopropane (3.09 mL, 30.9 mmol) was added and the resulting mixture was stirred at 25° C. for 12 h. The RM was quenched with sat. NH₄Cl (10 mL), diluted with water (40 mL) and extracted with EtOAc (3×50 mL). The combined organic layers were washed with brine (3×50 mL), dried over Na₂SO₄, filtered, and concentrated under reduced pressure. Purification by prep-HPLC (column: Phenomenex C₁₈, 250×70 mm, 10 μm; mobile phase: [water (NH₃H₂O+NH₄HCO₃)−ACN]; B: 35-60%, 20 min) gave the title compound as a colourless oil. Y=41%. ¹H NMR (400 MHz, DMSO-d₆) δ 7.69 (d, J=2 Hz, 1H), 6.43 (d, J=2 Hz, 1H), 4.56-4.45 (m, 1H), 1.38 (d, J=7 Hz, 6H).

Step 2. 1-(1-Isopropylpyrazol-3-yl)-2-methyl-propan-1-one. To isopropylmagnesium chloride lithium chloride (1 M in THF, 10.2 mL, 10.2 mmol) in THE (10 mL) at 0° C. was added a solution of 3-iodo-1-isopropyl-pyrazole (2.4 g, 10.2 mmol) in THE (10 mL). After 1 h, a solution of N-methoxy-N,-(2-dimethyl) propanamide (934 mg, 7.12 mmol) in THE (10 mL) was added at 25° C. The mixture was stirred at 25° C. for 12 h, quenched with sat. NH₄Cl (10 mL), diluted with water (20 mL) and extracted with EtOAc (3×20 mL). The combined organic layers were washed with brine (3×20 mL), dried over Na₂SO₄, filtered, and concentrated under reduced pressure. Purification by prep-TLC (SiO₂, 25% EtOAc in petroleum ether) gave the title compound as a yellow oil. Y=60%. ¹H NMR (400 MHz, DMSO-d₆) δ 7.90 (d, J=2 Hz, 1H), 6.69 (d, J=2 Hz, 1H), 4.66-4.55 (m, 1H), 3.67-3.55 (m, 1H), 1.45 (d, J=7 Hz, 6H), 1.09 (d, J=7 Hz, 6H).

Step 3. 1-(4-Iodo-1-isopropyl-pyrazol-3-yl)-2-methyl-propan-1-one. To a solution of 1-(1-isopropylpyrazol-3-yl)-2-methyl-propan-1-one (1.0 g, 5.55 mmol) in AcOH (10 mL) at 25° C. was added N-iodosuccinimide (1.87 g, 8.32 mmol). The RM was stirred at 25° C. for 12 h, diluted with water (10 mL) and extracted with EtOAc (3×10 mL). The combined organic layers were washed with brine (3×10 mL), dried over Na₂SO₄, filtered, and concentrated under reduced pressure. Purification by FCC (SiO₂, 10-25% EtOAc in petroleum ether) gave the title compound as a yellow oil. Y=88%. ¹H NMR (400 MHz, DMSO-d₆) δ 8.16 (s, 1H), 4.66-4.54 (m, 1H), 3.72-3.58 (m, 1H), 1.44 (d, J=7 Hz, 6H), 1.08 (d, J=7 Hz, 6H).

Step 4. Methyl 1-isopropyl-3-(2-methylpropanoyl)pyrazole-4-carboxylate. To a solution of 1-(4-iodo-1-isopropyl-pyrazol-3-yl)-2-methyl-propan-1-one (1.47 g, 4.80 mmol) in MeOH (8 mL) at 25° C. was added TEA (2.43 g, 24.0 mmol, 3.34 mL) and PdCl₂(dppf) (351 mg, 480 μmol). The suspension was degassed and purged with CO gas 3 times. The mixture was stirred under CO (50 Psi) at 70° C. for 12 h. The reaction mixture was filtered through a pad of Celite. The filtrate was diluted with water (15 mL) and extracted with EtOAc (3×15 mL). The combined organic layers were washed with brine (3×15 mL), dried over Na₂SO₄, filtered, and concentrated under reduced pressure. Purification by FCC (SiO₂, 10-50% EtOAc in petroleum ether gave the title compound as a yellow oil. Y=87%. ¹H NMR (400 MHz, DMSO-d₆) δ 8.42 (s, 1H), 4.66-4.54 (m, 1H), 3.71 (s, 3H), 3.43-3.38 (m, 1H), 1.44 (d, J=7 Hz, 6H), 1.08 (d, J=7 Hz, 6H).

Step 5. 2,7-Diisopropyl-5H-pyrazolo[3,4-d]pyridazin-4-one. To methyl 1-isopropyl-3-(2-methylpropanoyl)pyrazole-4-carboxylate (1.0 g, 4.20 mmol) in EtOH (10 mL) at 25° C. was added hydrazine hydrate (2.08 mL, 42.0 mmol). The resulting mixture was stirred at 80° C. for 12 h. The reaction mixture was filtered, and the filter cake was dried in vacuo to give the title compound as a white solid. Y=65%. ¹H NMR (400 MHz, DMSO-d₆) δ 11.88 (s, 1H), 8.73 (s, 1H), 4.89-4.73 (m, 1H), 3.31-3.25 (m, 1H), 1.52 (d, J=7 Hz, 6H), 1.30 (d, J=7 Hz, 6H).

### Intermediate C13. 2-Cyclopropyl-7-isopropyl-5H-pyrazolo [3,4-d]pyridazin-4-one

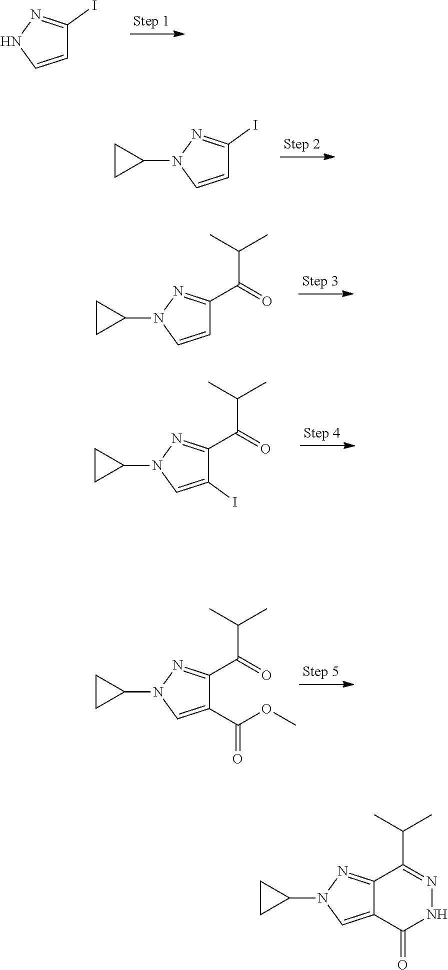 [SMILES: Ic1cc[nH]n1 ; Ic1ccn(C2CC2)n1 ; CC(C)C(=O)c1ccn(C2CC2)n1 ; CC(C)C(=O)c1nn(C2CC2)cc1I ; COC(=O)c1cn(C2CC2)nc1C(=O)C(C)C ; CC(C)c1n[nH]c(=O)c2cn(C3CC3)nc12]

Step 1. 1-Cyclopropyl-3-iodo-pyrazole. To a solution of 3-iodo-1H-pyrazole (5.0 g, 25.8 mmol) and cyclopropylboronic acid (4.43 g, 51.6 mmol) in DCE (50 mL) was added Na₂CO₃ (5.46 g, 51.6 mmol), 2-(2-pyridyl)pyridine (4.03 g, 25.8 mmol) and copper (II) diacetate (4.68 g, 25.8 mmol). The RM was stirred at 70° C. for 8 h. The reaction was cooled to 25° C. and AcOH (5 mL) was added. The resulting mixture was concentrated under vacuum. The residue was diluted with water (50 mL) and extracted with EtOAc (3×50 mL). The organic layer was dried over Na₂SO₄, filtered and concentrated in vacuo. Purification by FCC (SiO₂, 15% EtOAc in petroleum ether) gave the title compound as a colorless oil. Y=83%. ¹H NMR (400 MHz, DMSO-d₆) δ 7.68 (d, J=2 Hz, 1H), 6.42 (d, J=2 Hz, 1H), 3.78-3.66 (m, 1H), 1.05-0.98 (m, 2H), 0.97-0.89 (m, 2H).

Step 2. 1-(1-Cyclopropylpyrazol-3-yl)-2-methyl-propan-1-one. To a solution of isopropylmagnesium chloride lithium chloride (1 M, 21.4 mL, 21.4 mmol) in THE (50 mL) at 0° C. was added 1-cyclopropyl-3-iodo-pyrazole (5.0 g, 21.4 mmol). After 1 h, N-methoxy-N-(2-dimethyl)-propanamide (2.80 g, 21.4 mmol) in THE (50 mL) was added and the RM stirred at 25° C. for 12 h. The reaction mixture was quenched with sat. NH₄Cl (30 mL), diluted with water (20 mL) and extracted with EtOAc (3×30 mL). The combined organic layers were washed with brine (3×30 mL), dried over Na₂SO₄, filtered, and concentrated under reduced pressure. Purification by FCC (SiO₂, 25% EtOAc in petroleum ether) gave the title compound as a colourless liquid. Y=91%. ¹H NMR (400 MHz, CDCl₃) δ 7.45 (d, J=2 Hz, 1H), 6.74 (d, J=2 Hz, 1H), 3.73-3.62 (m, 2H), 1.20-1.16 (m, 8H), 1.10-1.04 (m, 2H).

Step 3. 1-(1-Cyclopropyl-4-iodo-pyrazol-3-yl)-2-methyl-propan-1-one. To 1-(1-cyclopropylpyrazol-3-yl)-2-methyl-propan-1-one (3.4 g, 19.1 mmol) in AcOH (35 mL) was added N-iodosuccinimide (6.44 g, 28.6 mmol). The RM was stirred at 25° C. for 8 hours, diluted with water (30 mL) and extracted with EtOAc (3×20 mL). The combined organic layers were washed with brine (30 mL), dried over Na₂SO₄, filtered, and concentrated under reduced pressure. Purification by FCC (SiO₂, 25% EtOAc in petroleum ether) gave the title compound as a brown oil. ¹H NMR (400 MHz, CDCl₃) δ 7.59 (s, 1H), 3.77-3.65 (m, 2H), 1.25-1.15 (m, 8H), 1.13-1.08 (m, 2H).

Step 4. Methyl 1-cyclopropyl-3-(2-methylpropanoyl)pyrazole-4-carboxylate. To a solution of 1-(1-cyclopropyl-4-iodo-pyrazol-3-yl)-2-methyl-propan-1-one (2.0 g, 6.58 mmol) in MeOH (50 mL) at 25° C. was added TEA (4.58 mL, 32.9 mmol) and PdCl₂ (dppf) (481 mg, 658 μmol). The RM was stirred under CO (gas, 50 psi) at 70° C. for 12 h. The reaction was filtered and the filtrate concentrated in vacuo. Purification by FCC (SiO₂, 20% EtOAc in petroleum ether) gave the title compound as a brown solid. Y=84%. ¹H NMR (400 MHz, MeOH-d₄) δ 8.23 (s, 1H), 3.82-3.79 (m, 1H), 3.79 (s, 3H), 3.58-3.45 (m, 1H), 1.20-1.17 (m, 2H), 1.15 (d, J=7 Hz, 6H), 1.12-1.06 (m, 2H).

Step 5. 2-Cyclopropyl-7-isopropyl-5H-pyrazolo[3, 4-d]pyridazin-4-one. To methyl 1-cyclopropyl-3-(2-methylpropanoyl)pyrazole-4-carboxylate (800 mg, 3.39 mmol) in EtOH (4 mL) at 25° C. was added hydrazine hydrate (1.68 mL, 33.9 mmol). The RM was stirred at 80° C. for 12 h. The reaction mixture was filtered and dried under reduced pressure to give the title compound as a green solid. Y=81% yield). ¹H NMR (400 MHz, DMSO-d₆) δ 11.90 (s, 1H), 8.74 (s, 1H), 4.09-4.00 (m, 1H), 3.30-3.20 (m, 1H), 1.30-1.23 (m, 8H), 1.14-1.07 (m, 2H).

### Intermediate C14. 2-(7-Cyclopropyl-1-isopropyl-4-oxo-pyrido[3,4-d]pyridazin-3-yl)acetic acid

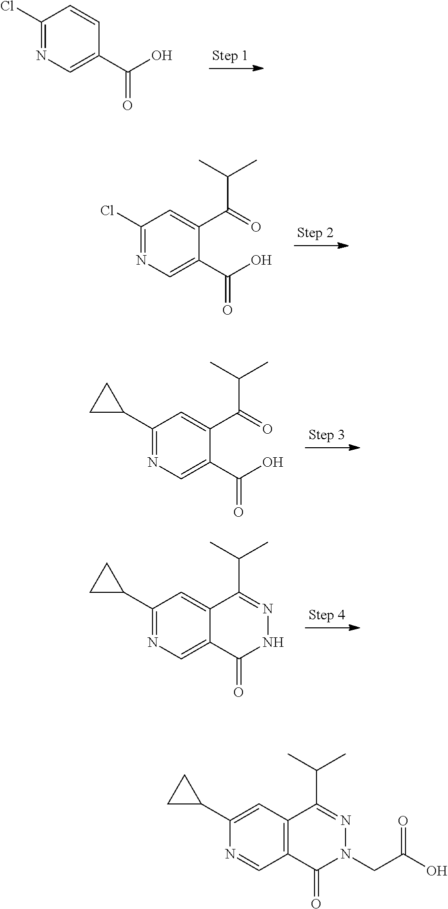

Step 1. 6-Chloro-4-(2-methylpropanoyl)pyridine-3-carboxylic acid. To a solution of n-BuLi (2.5 M, 50.8 mL, 127 mmol) in THE (130 mL) at −78° C. was added 2,2,6,6-tetramethylpiperidine (16.2 mL, 95.2 mmol) and the RM stirred at 0° C. for 30 mins. A solution of 6-chloropyridine-3-carboxylic acid (5 g, 31.7 mmol) in THE (10 mL) was added dropwise at −78° C. After addition, the mixture was stirred at this temperature for 1.5 h. N-methoxy-N-(2-dimethyl)propanamide (16.7 g, 127 mmol) was added and the mixture stirred at −78° C. for 3.5 h. The reaction mixture was quenched by addition of water (100 mL) at 0° C. and extracted with EtOAc (3×100 mL). The combined organic layers were washed with brine (2×100 mL), dried over Na₂SO₄, filtered, and concentrated under reduced pressure. FCC (SiO₂, 25-100% EtOAc in petroleum ether) gave the title compound as a white solid. Y=14%.

Step 2. 6-Cyclopropyl-4-(2-methylpropanoyl)pyridine-3-carboxylic acid. A mixture of 6-chloro-4-(2-methylpropanoyl)pyridine-3-carboxylic acid (1.0 g, 4.39 mmol), cyclopropylboronic acid (1.13 g, 13.2 mmol), K₃PO₄ (1.96 g, 9.22 mmol) and PdCl₂(dppf) (161 mg, 220 μmol) in dioxane (25 mL) was stirred at 100° C. for 8 h under N₂. The RM was concentrated under reduced pressure, then diluted water (30 mL) and extracted with DCM (3×30 mL). The combined organic layers were washed with brine (2×30 mL), dried over Na₂SO₄, filtered, and concentrated under reduced pressure. Purification by FCC (SiO₂, 25-100% EtOAc in petroleum ether) gave the title compound as a yellow oil. Y=98%.

Step 3. 7-Cyclopropyl-1-isopropyl-3H-pyrido[3,4-d]pyridazin-4-one. To 6-cyclopropyl-4-(2-methylpropanoyl)pyridine-3-carboxylic acid (0.43 g, 1.84 mmol) in EtOH (3 mL) at 25° C. was added hydrazine hydrate (1.83 mL, 36.9 mmol). The mixture was stirred at 80° C. for 12 h. The reaction was cooled to 25° C., filtered and dried to give the title compound as a white solid. Y=47%. ¹H NMR (400 MHz, DMSO-d₆) δ 9.28 (s, 1H), 7.84 (s, 1H), 3.57-3.51 (m, 1H), 2.45-2.37 (m, 1H), 1.26 (d, J=7 Hz, 6H), 1.12-1.08 (m, 4H).

Step 4. 2-(7-Cyclopropyl-1-isopropyl-4-oxo-pyrido[3,4-d]pyridazin-3-yl)acetic acid. To a solution of 2-bromoacetic acid (80.1 μL, 155 mg) in THE (1 mL) was added LiO^{t}Bu (267 μL, 2.97 mmol) and 7-cyclopropyl-1-isopropyl-3H-pyrido[3,4-d]pyridazin-4-one (0.17 g, 741 μmol). The RM was stirred at 80° C. for 3 h, cooled and 2 M HCl was added until pH=4. The resulting mixture was diluted with water (2 mL) and extracted with EtOAc (3×3 mL). The combined organic layers were washed with brine (2×3 mL), dried over Na₂SO₄, filtered, and concentrated under reduced pressure to give the title compound as a white solid. Y=94%. ¹H NMR (400 MHz, DMSO-d₆) δ 13.00 (br. s, 1H), 9.29 (s, 1H), 7.88 (s, 1H), 4.76 (s, 2H), 3.63-3.51 (m, 1H), 2.45-2.37 (m, 1H), 1.25 (d, J=7 Hz, 6H), 1.12-1.07 (m, 4H).

### Intermediate C15. 2-(2,7-Diethyl-4-oxo-pyrazolo[3,4-d]pyridazin-5-yl)acetic acid

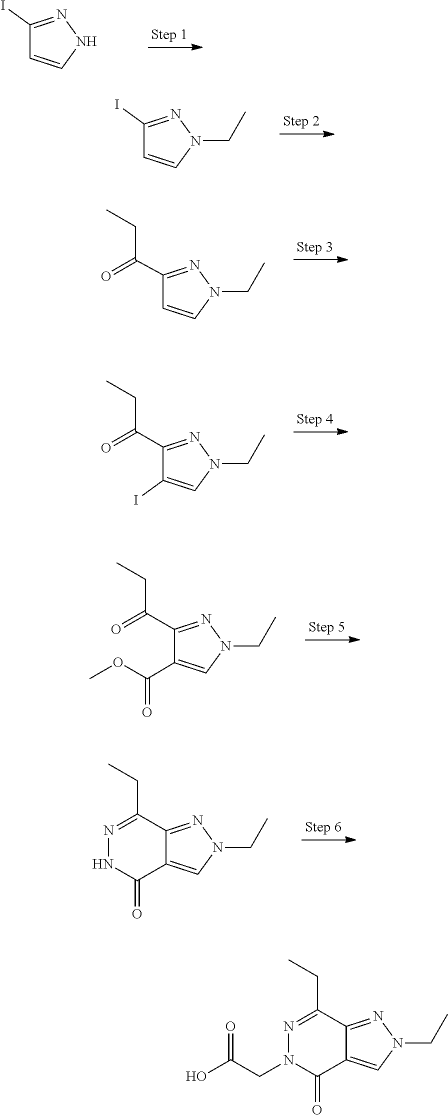

Step 1. I-Ethyl-3-iodo-pyrazole. To a solution of 3-iodo-1H-pyrazole (19 g, 98.0 mmol) in DMF (100 mL) was added Cs₂CO₃ (95.7 g, 294 mmol). After stirring for 30 mins at 25° C., iodoethane (15.7 mL, 196 mmol) was added and the RM stirred for 2 h. The reaction mixture was diluted with water (100 mL) and extracted with EtOAc (3×100 mL). The combined organic layers were washed with brine (3×100 mL), dried over Na₂SO₄, filtered, and concentrated under reduced pressure. Purification by FCC (SiO₂, 10-20% EtOAc in petroleum ether) gave the title compound as a pale yellow oil. Y=52%. ¹H NMR (400 MHz, DMSO-d₆) δ 7.66 (d, J=2 Hz, 1H), 6.43 (d, J=2 Hz, 1H), 4.13 (q, J=7 Hz, 2H), 1.34 (t, J=7 Hz, 3H).

Step 2. 1-(1-Ethylpyrazol-3-yl)-2-methyl-propan-1-one. To a solution of isopropylmagnesium chloride lithium chloride (1 M, 13.5 mL, 13.5 mmol) in THE (20 mL) was added a solution of 1-ethyl-3-iodo-pyrazole (3.0 g, 13.5 mmol) in THE (7 mL) at 0° C. The RM was stirred at 25° C. for 1 h then a solution of N-methoxy-N,2-dimethyl-propanamide (1.24 g, 9.46 mmol) in THE (10 mL) was added. The RM was stirred at 25° C. for 12 h under N₂, quenched with sat. NH₄Cl (10 mL), diluted with water (30 mL) and extracted with EtOAc (3×30 mL). The combined organic layers were washed with brine (3×20 mL), dried over Na₂SO₄, filtered, and concentrated under reduced pressure. Purification by FCC (SiO₂, 10-20% EtOAc in petroleum ether) gave the title compound as a yellow oil. Y=58%. ¹H NMR (400 MHz, DMSO-d₆) δ 7.87 (d, J=2 Hz, 1H), 6.69 (d, J=2 Hz, 1H), 4.22 (q, J=7 Hz, 2H), 3.68-3.56 (m, 1H), 1.40 (t, J=7 Hz, 3H), 1.09 (d, J=7 Hz, 6H).

Step 3. 1-(1-Ethyl-4-iodo-pyrazol-3-yl)-2-methyl-propan-1-one. To a solution of 1-(1-ethylpyrazol-3-yl)-2-methyl-propan-1-one (1.3 g, 7.82 mmol) in AcOH (15 mL) at 25° C. was added NIS (2.64 g, 11.7 mmol). The RM was stirred for 12 h, diluted with water (15 mL) and extracted with EtOAc (3×15 mL). The combined organic layers were washed with brine (3×15 mL), dried over Na₂SO₄, filtered, and concentrated under reduced pressure. Purification by FCC (SiO₂, 10-25% EtOAc in petroleum ether) gave the title compound as a yellow solid. Y=92%. ¹H NMR (400 MHz, DMSO-d₆) δ 8.11 (s, 1H), 4.22 (q, J=7 Hz, 2H), 3.70-3.57 (m, 1H), 1.40 (t, J=7 Hz, 3H), 1.08 (d, J=7 Hz, 6H).

Step 4. Methyl 1-ethyl-3-propanoyl-pyrazole-4-carboxylate. To a solution of 1-(1-ethyl-4-iodo-pyrazol-3-yl)propan-1-one (1.0 g, 3.60 mmol) in MeOH (10 mL) at 25° C. was added TEA (2.50 mL, 18.0 mmol) and PdCl₂(dppf) (263 mg, 360 μmol). The RM was stirred under CO (gas, 50 Psi) at 70° C. for 12 h, then concentrated under reduced pressure. The residue was filtered through a pad of Celite, then the filtrate was diluted with water (15 mL) and extracted with EtOAc (3×15 mL). The combined organic layers were washed with brine (3×15 mL), dried over Na₂SO₄, filtered, and concentrated under reduced pressure. Purification by FCC (SiO₂, 10-50% EtOAc in petroleum ether) gave the title compound as a yellow oil. Y=93%. ¹H NMR (400 MHz, DMSO-d₆) δ 8.39 (s, 1H), 4.21 (q, J=7 Hz, 2H), 3.72 (s, 3H), 2.92 (q, J=7 Hz, 2H), 1.40 (t, J=7 Hz, 3H), 1.05 (t, J=7 Hz, 3H).

Step 5. 2,7-Diethyl-5H-pyrazolo[3,4-d]pyridazin-4-one. To a solution of methyl 1-ethyl-3-propanoyl-pyrazole-4-carboxylate (0.70 g, 3.33 mmol) in EtOH (7 mL) at 25° C. was added hydrazine hydrate (1.65 mL, 33.3 mmol). The RM was stirred at 80° C. for 12 h, filtered and dried under reduced pressure to give the title compound as a white solid. Y=78%. ¹H NMR (400 MHz, DMSO-d₆) δ 11.90 (s, 1H), 8.69 (s, 1H), 4.41 (q, J=7 Hz, 2H), 2.81 (q, J=7 Hz, 2H), 1.47 (t, J=7 Hz, 3H), 1.26 (t, J=7 Hz, 3H).

Step 6. 2-(2,7-Diethyl-4-oxo-pyrazolo[3,4-d]pyridazin-5-yl)acetic acid. To a solution of 2,7-diethyl-5H-pyrazolo[3,4-d]pyridazin-4-one (175 mg, 910 μmol) in THE (3 mL) at 25° C. was added lithium tert-butoxide (328 μL, 3.64 mmol) and 2-bromoacetic acid (98.3 μL, 1.37 mmol). The RM was stirred at 80° C. for 3 h under N₂, then diluted with water (1 mL) and washed with EtOAc (3×1 mL). 2 M HCl was added dropwise until pH=4-5 and the aqueous phase was extracted with EtOAc (3×1 mL). The combined organic layers were washed with brine (3×1 mL), dried over Na₂SO₄, filtered, and concentrated under reduced pressure to give the title compound as a white solid. Y=79%. ¹H NMR (400 MHz, DMSO-d₆) δ 12.84 (br. s, 1H), 8.81 (s, 1H), 4.70 (s, 2H), 4.43 (q, J=7 Hz, 2H), 2.84 (q, J=7 Hz, 2H), 1.48 (t, J=7 Hz, 3H), 1.27 (t, J=7 Hz, 3H).

### Intermediate C16. 2-Ethyl-7-isopropyl-5H-furo[2,3-d]pyridazin-4-one

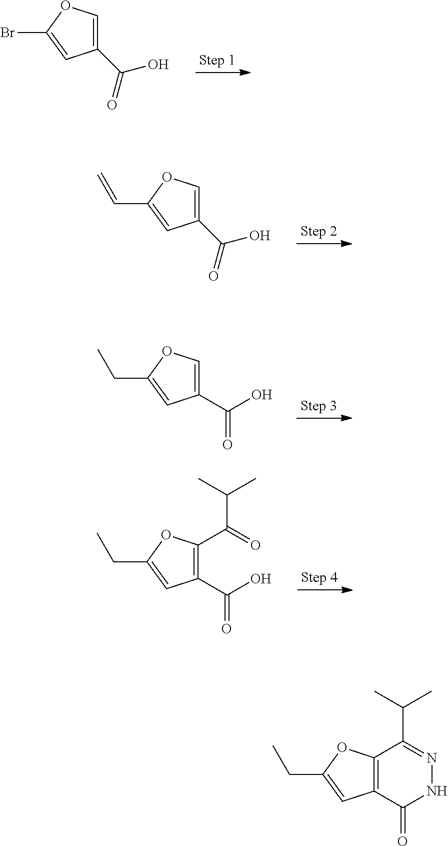

Step 1. 5-Vinylfuran-3-carboxylic acid. To a solution of 5-bromofuran-3-carboxylic acid (500 mg, 2.62 mmol) in dioxane (10 mL) and water (2 mL) at 25° C. was added Na₂CO₃ (555 mg, 5.24 mmol), Pd C₁₂(dppf) (383 mg, 524 μmol) and 4,4,5,5-tetramethyl-2-vinyl-1,3,2-dioxaborolane (666 μL, 3.93 mmol). The mixture was stirred at 80° C. for 3 h. The pH was adjusted to ˜3 by addition of 1M HCl. Water (3 mL) was added and the solution extracted with ethyl acetate (3×3 mL). The combined organic layers were washed with brine (5 mL), dried over Na₂SO₄, filtered, and concentrated under reduced pressure to give the title compound as a white solid. Y=83% yield.

Step 2. 5-Ethylfuran-3-carboxylic acid. To a solution of 5-vinylfuran-3-carboxylic acid (270 mg, 1.95 mmol) in MeOH (5 mL) at 25° C. under N₂ was added Pd/C (200 mg, 10% on carbon, 50% in water). The RM was stirred under H₂ (15 psi) for 1 h. The reaction was filtered and filtrate was adjusted to pH=8 with saturated NaHCO₃ (2 mL). The mixture was washed with ethyl acetate (2 mL). The aqueous phase was adjusted to pH=2 with 1 M HCl and extracted with ethyl acetate (3×1 mL). The organic layers were washed with brine (1×1 mL), dried over Na₂SO₄, filtered, and concentrated under reduced pressure to give the title compound as a brown solid. Y=91%. ¹H NMR (400 MHz, DMSO-d₆) δ 12.55 (br. s, 1H), 8.11 (s, 1H), 6.33 (s, 1H), 2.62 (q, J=7 Hz, 2H), 1.17 (t, J=7 Hz, 3H).

Step 3. 5-Ethyl-2-(2-methylpropanoyl)furan-3-carboxylic acid. To a solution of 5-ethylfuran-3-carboxylic acid (50 mg, 357 μmol) in THE (1 mL) at −70° C. under N₂ was added LDA (2 M in THF, 535 μL, 1.07 mmol). The mixture was stirred at −70° C. for 30 mins then N-methoxy-N-(2-dimethyl)propanamide (93.6 mg, 714 μmol) was added. The RM was stirred at −70° C. for 1 h, quenched with sat. NH₄Cl (3 mL) at 0° C. and adjusted to pH=3 with 1 M HCl. The resulting mixture was diluted with water (1 mL) and extracted with ethyl acetate (3×1 mL). The combined organic layers were washed with brine (3 mL), dried over Na₂SO₄, filtered, and concentrated under reduced pressure. Purification by prep-TLC (SiO₂, 1:1 petroleum ether:ethyl acetate) gave the title compound as a white solid. Y=20%. ¹H NMR (400 MHz, DMSO-d₆) δ 6.64 (s, 1H), 3.53-3.48 (m, 1H), 2.73 (d, J=7 Hz, 2H), 1.23-1.19 (m, 3H), 1.11 (d, J=7 Hz, 6H).

Step 4. 2-Ethyl-7-isopropyl-5H-furo[2,3-d]pyridazin-4-one. To a solution of 5-ethyl-2-(2-methylpropanoyl) furan-3-carboxylic acid (110 mg, 523 μmol) in EtOH (3 mL) at 25° C. was added hydrazine hydrate (519 μL, 10.5 mmol). The mixture was stirred at 70° C. for 2 h then concentrated under reduced pressure to give the title compound as a white solid. Y=93%.

### Intermediate C17. 2-Ethyl-4-isopropyl-6H-thieno [2,3-d]pyridazin-7-one

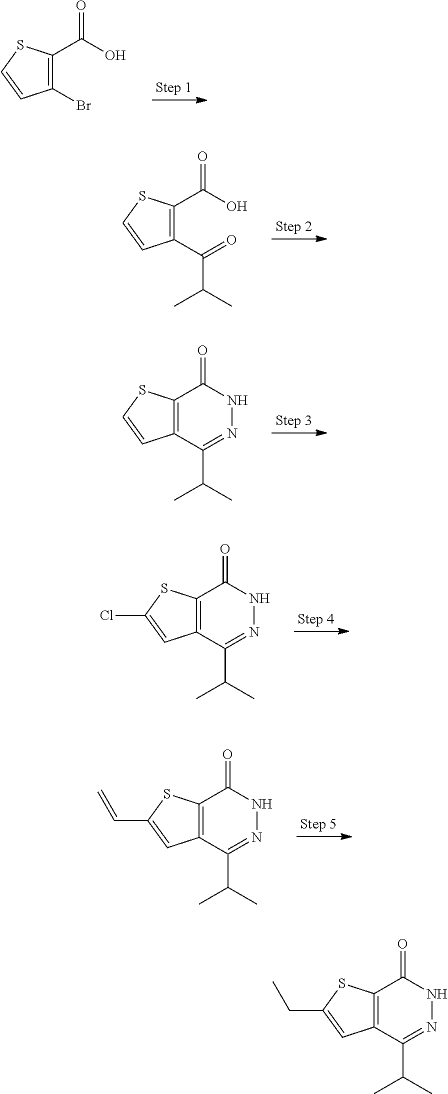

Step 1. 3-(2-Methylpropanoyl)thiophene-2-carboxylic acid. To a solution of 3-bromothiophene-2-carboxylic acid (2.0 g, 9.66 mmol) in THE (20 mL) at −78° C. was added n-BuLi (2.5 M, 7.73 mL, 19.3 mmol). After 30 mins, N-methoxy-N-(2-dimethyl)propanamide (1.52 g, 11.6 mmol) in THE (3 mL) was added and the RM was stirred at 25° C. for 1 h. Addition of 2 M HCl (10 mL) adjusted the RM to pH 4-5. The resulting mixture was diluted with water (5 mL) and extracted with ethyl acetate (3×20 mL). The combined organic layers were washed with brine (30 mL), dried over anhydrous Na₂SO₄, filtered, and concentrated under reduced pressure. Purification by FCC (SiO₂, 10-25% EtOAc in petroleum ether) gave the title compound as a white solid. Y=52%.

Step 2. 4-Isopropyl-6H-thieno[2,3-d]pyridazin-7-one. To a solution of 3-(2-methylpropanoyl) thiophene-2-carboxylic acid (0.50 g, 2.52 mmol) in EtOH (5 mL) at 25° C. was added hydrazine hydrate (2.50 mL, 50.4 mmol). The mixture was stirred at 80° C. for 2 h, concentrated in vacuo, then filtered and dried to give the title compound as a white solid. Y=71%.

Step 3. 2-Chloro-4-isopropyl-6H-thieno [2,3-d]pyridazin-7-one. To 4-isopropyl-6H-thieno[2,3-d]pyridazin-7-one (350 mg, 1.80 mmol) in THE (4 mL) at −78° C. was added 2 M LDA in THE (2.25 mL, 4.50 mmol). After stirring for 30 mins, 1,1,1,2,2,2-hexachloroethane (204 μL, 1.80 mmol) was added at −25° C. The RM was stirred at 0° C. for 2 h, quenched with water (3 mL) at 0° C. and the resulting mixture concentrated under reduced pressure. The residue was diluted with water (3 mL) and extracted with EtOAc (3×2 mL). The combined organic layers were washed with brine (2×2 mL), dried over Na₂SO₄, filtered, and concentrated under reduced pressure. Purification by FCC (SiO₂, 20% EtOAc in petroleum ether) gave the title compound as a white solid. Y=39%. ¹H NMR (400 MHz, DMSO-d₆) δ 12.87 (s, 1H), 7.86 (s, 1H), 3.30-3.27 (m, 1H), 1.24 (d, J=7 Hz, 6H).

Step 4. 4-Isopropyl-2-vinyl-6H-thieno [2,3-d]pyridazin-7-one. To a solution of 2-chloro-4-isopropyl-6H-thieno[2,3-d]pyridazin-7-one (200 mg, 875 μmol) in THE (4 mL) and water (0.8 mL) at 25° C. was added 4,4,5,5-tetramethyl-2-vinyl-1,3,2-dioxaborolane (445 μL, 2.62 mmol), Na₂CO₃ (463 mg, 4.37 mmol) and Pd(PPh₃)₄ (101 mg, 87.5 μmol). The RM was stirred at 80° C. for 12 h, diluted with water (5 mL) and extracted with ethyl acetate (3×5 mL). The combined organic layers were washed with brine (5 mL), dried over anhydrous Na₂SO₄, filtered, and concentrated under reduced pressure. Purification by FCC (SiO₂, 25% EtOAc in petroleum ether) gave the title compound as a white solid. Y=57%.

Step 5. 2-Ethyl-4-isopropyl-6H-thieno [2,3-d]pyridazin-7-one. To a solution of 4-isopropyl-2-vinyl-6H-thieno [2,3-d]pyridazin-7-one (70 mg, 318 μmol) in EtOH (2 mL) was added Pd/C (70 mg, 10% Pd on carbon, 50% in water) and the mixture stirred under H₂ (15 psi) at 25° C. for 1 h. The solution was filtered and the filtrate was concentrated under vacuum to give the title compound as a yellow oil. Y=quantitative.

### Intermediate C18. 2-(2-Cyclopropyl-8-isopropyl-5-oxo-pyrido[2,3-d]pyridazin-6-yl)acetic acid

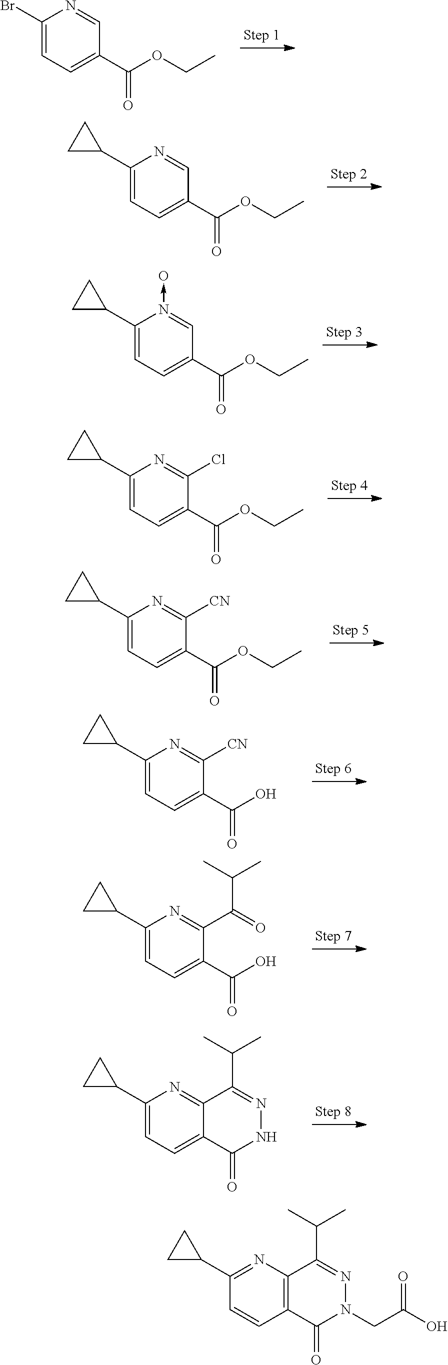

Step 1. Ethyl 6-cyclopropylpyridine-3-carboxylate. A mixture of ethyl 6-bromopyridine-3-carboxylate (13.4 g, 58.1 mmol), cyclopropyl boronic acid (12.5 g, 145 mmol), K₃PO₄ (25.9 g, 122 mmol) and PdCl₂(dppf) (2.13 g, 2.91 mmol) in dioxane (150 mL) was stirred at 100° C. for 8 h under N₂. The RM was concentrated under reduced pressure, diluted with water (100 mL) and extracted with DCM (3×100 mL). The combined organic layers were washed with brine (100 mL) and concentrated under reduced pressure. Purification by FCC (SiO₂, 0-30% EtOAc in petroleum ether) gave the title compound as a yellow oil. Y=45%. ¹H NMR (400 MHz, MeOH-d₄) δ 8.91 (dd, J=1, 2 Hz, 1H), 8.16 (dd, J=2, 8 Hz, 1H), 7.32 (dd, J=1, 8 Hz, 1H), 4.37 (q, J=7 Hz, 2H), 2.20-2.11 (m, 1H), 1.38 (t, J=7 Hz, 3H), 1.13-1.03 (m, 4H).

Step 2. Ethyl 6-cyclopropyl-1-oxido-pyridin-1-ium-3-carboxylate. To a solution of ethyl 6-cyclopropylpyridine-3-carboxylate (5.0 g, 26.1 mmol) in DCM (50 mL) at 25° C. was added m-CPBA (10.6 g, 52.3 mmol). The mixture was stirred at 25° C. for 1 h and quenched with sat. Na₂S₂O₃ (50 mL). Water (50 mL) was added and the solution extracted with DCM (3×100 mL). The combined organic layers were washed with brine (2×100 mL), dried over Na₂SO₄, filtered, and concentrated under reduced pressure. Purification by FCC (SiO₂, 10-100% EtOAc in petroleum ether) gave the title compound as a white solid. Y=92%.

Step 3. Ethyl 2-chloro-6-cyclopropyl-pyridine-3-carboxylate. Ethyl 6-cyclopropyl-1-oxido-pyridin-1-ium-3-carboxylate (4.8 g, 23.2 mmol) in POCl₃ (50 mL, 538 mmol) was stirred at 100° C. for 12 h. The mixture was cooled to 25° C., then poured into water (200 mL). The resulting mixture was extracted with EtOAc (3×200 mL). The combined organic layers were washed with brine (2×100 mL), dried over Na₂SO₄, filtered, and concentrated under reduced pressure. Purification by FCC (SiO₂, 10-30% EtOAc in petroleum ether) gave the title compound as a yellow oil. Y=93%. ¹H NMR (400 MHz, MeOH-d₄) δ 8.04 (d, J=8 Hz, 1H), 7.27 (d, J=8 Hz, 1H), 4.38-4.33 (q, J=7 Hz, 2H), 2.17-2.05 (m, 1H), 1.37 (t, J=7 Hz, 3H), 1.10-1.03 (m, 4H).

Step 4. Ethyl 2-cyano-6-cyclopropyl-pyridine-3-carboxylate. To a solution of ethyl 2-chloro-6-cyclopropyl-pyridine-3-carboxylate (2.0 g, 8.86 mmol) in DMA (16 mL) at 25° C. was added Zn(CN)₂ (1.13 mL, 17.72 mmol), Zinc (69.5 mg, 1.06 mmol), Pd₂(dba)₃ (81.2 mg, 88.6 μmol) and DPPF (98.3 mg, 177 μmol). The RM was stirred at 120° C. for 2 h under N₂, diluted with water (30 mL) and extracted with EtOAc (3×30 mL). The combined organic layers were washed with brine (2×30 mL), dried over Na₂SO₄, filtered, and concentrated under reduced pressure. Purification by FCC (SiO₂, 10-25% EtOAc in petroleum ether) gave the title compound as a yellow oil. Y=94%. ¹H NMR (400 MHz, DMSO-d₆) δ 8.28 (d, J=8 Hz, 1H), 7.74 (d, J=8 Hz, 1H), 4.37 (q, J=7 Hz, 2H), 2.33-2.26 (m, 1H), 1.34 (t, J=7 Hz, 3H), 1.16-1.10 (m, 2H), 1.05-1.00 (m, 2H).

Step 5. 2-Cyano-6-cyclopropyl-pyridine-3-carboxylic acid. To a solution of ethyl 2-cyano-6-cyclopropyl-pyridine-3-carboxylate (1.8 g, 8.32 mmol) in water (3 mL) and THE (15 mL) at 25° C. was added LiOH·H₂O (699 mg, 16.7 mmol). The mixture was stirred at 25° C. for 2 h, concentrated under reduced pressure, diluted with water (10 mL) and lyophilized to give the Li salt of the title compound as a white solid. Y=quantitative. LCMS (ESI): m/z: [M+H]⁺=189.1.

Step 6. 6-Cyclopropyl-2-(2-methylpropanoyl)pyridine-3-carboxylic acid. To 2-cyano-6-cyclopropyl-pyridine-3-carboxylic acid (0.80 g, 4.10 mmol) in THE (8 mL) at 0° C. was added isopropylmagnesium chloride (2 M in THF, 2.05 mL, 4.1 mmol). The RM was stirred at 25° C. for 5 h, quenched with water (5 mL) at 0° C. and extracted with EtOAc (3×10 mL). The combined organic layers were washed with brine (10 mL), dried over Na₂SO₄, filtered, and concentrated under reduced pressure. Purification by FCC (SiO₂, 5-50% EtOAc in petroleum ether) gave the title compound as a white solid. Y=52%.

Step 7. 2-Cyclopropyl-8-isopropyl-6H-pyrido[2,3-d]pyridazin-5-one. To 6-cyclopropyl-2-(2-methylpropanoyl)pyridine-3-carboxylic acid (0.50 g, 2.14 mmol) in EtOH (5 mL) at 25° C. was added hydrazine hydrate (2.13 mL, 42.9 mmol). The RM was stirred at 80° C. for 12 h, cooled to 25° C. gradually, filtered and dried to give the title compound as a white solid. Y=37%. ¹H NMR (400 MHz, DMSO-d₆) δ 12.56 (s, 1H), 8.40 (d, J=8 Hz, 1H), 7.76 (d, J=8 Hz, 1H), 3.78-3.68 (m, 1H), 2.43-2.28 (m, 1H), 1.25 (d, J=7 Hz, 6H), 1.22-1.00 (m, 4H).

Step 8. 2-(2-Cyclopropyl-8-isopropyl-5-oxo-pyrido[2,3-d]pyridazin-6-yl)acetic acid. To 2-cyclopropyl-8-isopropyl-6H-pyrido[2,3-d]pyridazin-5-one (0.17 g, 741 μmol) in THE (1.7 mL) at 25° C. was added lithium tert-butoxide (267 μL, 2.97 mmol) and 2-bromoacetic acid (80.1 μL, 1.11 mmol). The RM was stirred at 80° C. for 3 h, cooled and the pH adjusted to pH 4 with aqueous 2 M HCl. The solution was diluted with water (3 mL) and extracted with EtOAc (3×3 mL). The combined organic layers were washed with brine (2×3 mL), dried over Na₂SO₄, filtered and concentrated under reduced pressure to give the title compound as a white solid. Y=94%. ¹H NMR (400 MHz, DMSO-d₆) δ 12.54 (br. s, 1H), 8.41 (d, J=8 Hz, 1H), 7.78 (d, J=8 Hz, 1H), 4.76 (s, 2H), 3.72 (q, J=7 Hz, 1H), 2.40-2.33 (m, 1H), 1.24 (d, J=7 Hz, 6H), 1.17-1.10 (m, 4H).

### Intermediate C19. 2-Bromo-4-isopropyl-6H-thieno[2,3-d]pyridazin-7-one

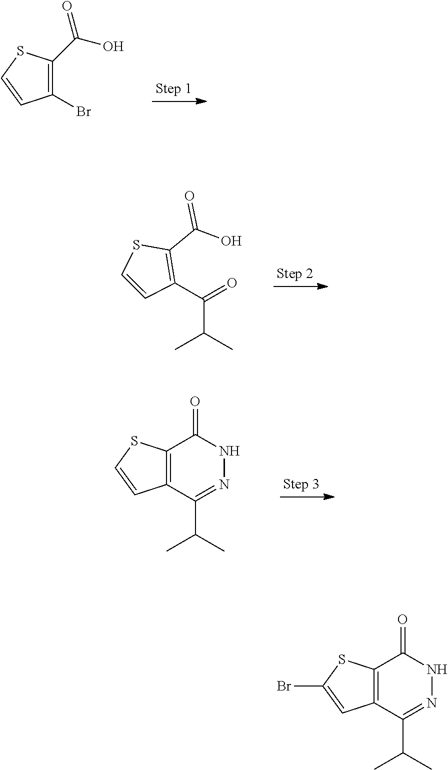

Step 1. 3-(2-Methylpropanoyl)thiophene-2-carboxylic acid. To a solution of 3-bromothiophene-2-carboxylic acid (3.0 g, 14.5 mmol) in THE (30 mL) at −78° C. was added n-BuLi (2.5 M in THF, 11.6 mL). After stirring for 30 min, N-methoxy-N-(2-dimethyl)propanamide (2.28 g, 17.4 mmol) was added−78° C. under N₂. The RM was stirred at 25° C. for 1 h, then quenched with sat. NH₄Cl (20 mL) at 0° C. Water (20 mL) was added and the solution was extracted with EtOAc (3×30 mL). The combined organic layers were washed with brine (15 mL), dried over Na₂SO₄, filtered, and concentrated under reduced pressure. Purification by FCC (SiO₂, 50-100% EtOAc in petroleum ether) gave the title compound as a yellow solid. Y=97%. ¹H NMR (400 MHz, DMSO-d₆) δ 13.44 (br. s, 1H), 7.98 (d, J=5 Hz, 1H), 7.18 (d, J=5 Hz, 1H), 3.15-3.10 (m, 1H), 1.06 (d, J=7 Hz, 6H).

Step 2. 4-Isopropyl-6H-thieno[2,3-d]pyridazin-7-one. To a solution of 3-(2-methylpropanoyl)thiophene-2-carboxylic acid (2.8 g, 14.1 mmol) in EtOH (30 mL) at 25° C. was added hydrazine hydrate (14.0 mL, 282 mmol). The RM was stirred at 80° C. for 12 h then cooled and filtered to give the title compound as a white solid. Y=55%. ¹H NMR (400 MHz, DMSO-d₆) δ 12.67 (br., 1H), 8.24 (d, J=5 Hz, 1H), 7.70 (d, J=5 Hz, 1H), 3.40-3.34 (m, 1H), 1.27 (d, J=7 Hz, 6H).

Step 3. 2-Bromo-4-isopropyl-6H-thieno[2,3-d]pyridazin-7-one. To a solution of 4-isopropyl-6H-thieno[2,3-d]pyridazin-7-one (1.3 g, 6.69 mmol) in THE (15 mL) at −78° C. was added LDA (2 M in THF, 8.37 mL, 16.7 mmol). After stirring for 30 mins, 1,2-dibromo-1,1,2,2-tetrachloro-ethane (965 μL, 8.03 mmol) was added at −78° C. The RM was stirred at 25° C. for 2 h under N₂. The RM was quenched by addition of water (15 mL) at 0° C. and extracted with EtOAc (3×25 mL). The combined organic layers were washed with brine (8 mL), dried over Na₂SO₄, filtered and concentrated under reduced pressure. Purification by FCC (SiO₂, 30-100% EtOAc in petroleum ether) gave the title compound as a yellow solid. Y=82%.

### Intermediate C20. 2-Cyclopropyl-8-ethyl-6H-pyrido[2,3-d]pyridazin-5-one

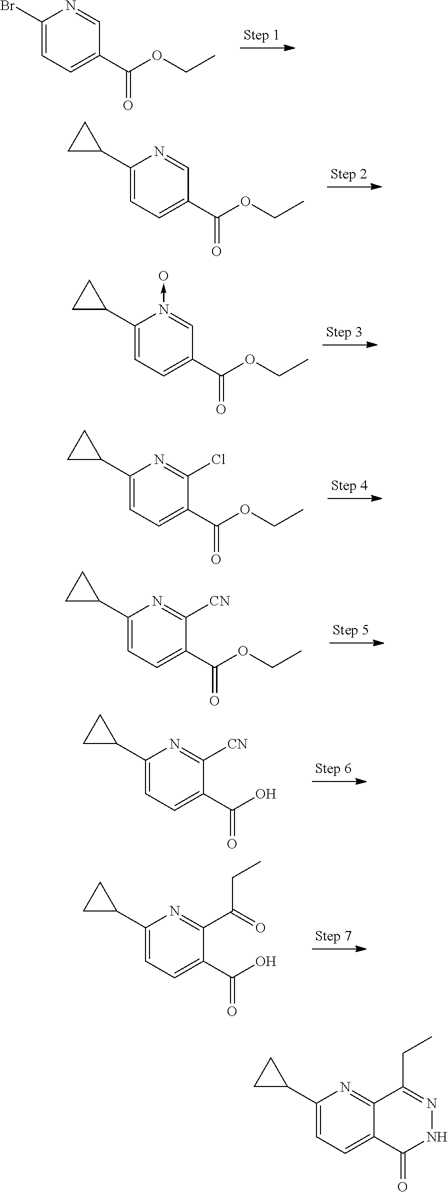

Step 1. Ethyl 6-cyclopropylpyridine-3-carboxylate. A mixture of ethyl 6-bromopyridine-3-carboxylate (13.4 g, 58.1 mmol), cyclopropylboronic acid (12.5 g, 145 mmol), K₃PO₄ (25.9 g, 122 mmol) and PdCl₂(dppf) (2.13 g, 2.91 mmol) in dioxane (150 mL) was stirred at 100° C. for 8 h under N₂. The RM was concentrated under reduced pressure, diluted with water (100 mL) and the resulting mixture was extracted with DCM (3×100 mL). The combined organic layers were washed with brine (100 mL) and concentrated under reduced pressure. Purification by FCC (SiO₂, 0-30% EtOAc in petroleum ether) gave the title compound as a yellow oil. Y=45%. ¹H NMR (400 MHz, MeOH-d₄) δ 8.91 (dd, J=1, 2 Hz, 1H), 8.16 (dd, J=2, 8 Hz, 1H), 7.32 (dd, J=1, 8 Hz, 1H), 4.37 (q, J=7 Hz, 2H), 2.20-2.11 (m, 1H), 1.38 (t, J=7 Hz, 3H), 1.13-1.03 (m, 4H).

Step 2. Ethyl 6-cyclopropyl-1-oxidopyridinium-3-carboxylate. To a solution of ethyl 6-cyclopropylpyridine-3-carboxylate (5 g, 26.15 mmol) in DCM (50 mL) was added m-CPBA (10.62 g, 52.29 mmol) at 25° C. The mixture was stirred at 25° C. for 1 hr. The reaction mixture was quenched by addition of saturated Na₂S₂O₃ (50 mL) at 25° C. The resulting mixture was diluted with H₂O (50 mL) and extracted with DCM (100 mL×3). The combined organic layers were washed with brine (100 mL×2), dried over Na₂SO₄, filtered, and concentrated under reduced pressure. FCC (SiO₂, 10-100% EtOAc in Petroleum ether gave the title compound as a white solid. Y=92%.

Step 3. Ethyl 2-chloro-6-cyclopropyl-pyridine-3-carboxylate. Ethyl 6-cyclopropyl-1-oxido-pyridinium-3-carboxylate (4.8 g, 23.2 mmol) in POCl₃ (50 mL, 538 mmol) was stirred at 100° C. for 12 hrs. The mixture was cooled down to 25° C., then poured into water (200 mL). The resulting mixture was extracted with EtOAc (3×200 mL). The combined organic layers were washed with brine (2×100 mL), dried over Na₂SO₄, filtered and concentrated under reduced pressure. Purification by FCC (SiO₂, 10-30% EtOAc in petroleum ether) gave the title compound as a yellow oil. Y=93%. ¹H NMR (400 MHz, MeOH-d₄) δ 8.04 (d, J=8 Hz, 1H), 7.27 (d, J=8 Hz, 1H), 4.38-4.33 (q, J=7 Hz, 2H), 2.17-2.05 (m, 1H), 1.37 (t, J=7 Hz, 3H), 1.10-1.03 (m, 4H).

Step 4. Ethyl 2-cyano-6-cyclopropyl-pyridine-3-carboxylate. To a solution of ethyl 2-chloro-6-cyclopropylpyridine-3-carboxylate (2.0 g, 8.86 mmol) in DMA (16 mL) was added Zn(CN)₂ (1.13 mL, 17.7 mmol), Zn (69.5 mg, 1.06 mmol), Pd₂(dba)₃ (81.2 mg, 88.6 μmol) and DPPF (98.3 mg, 177 μmol). The mixture was stirred at 120° C. for 2 h under N₂. The reaction mixture was diluted with water (30 mL) and extracted with EtOAc (3×30 mL). The combined organic layers were washed with brine (2×30 mL), dried over Na₂SO₄, filtered and concentrated under reduced pressure. Purification by FCC (SiO₂, 10-25% EtOAc in petroleum ether) gave the title compound as a yellow oil. Y=94%. ¹H NMR (400 MHz, DMSO-d₆) δ 8.28 (d, J=8 Hz, 1H), 7.74 (d, J=8 Hz, 1H), 4.37 (q, J=7 Hz, 2H), 2.33-2.26 (m, 1H), 1.34 (t, J=7 Hz, 3H), 1.16-1.10 (m, 2H), 1.05-1.00 (m, 2H).

Step 5. 2-Cyano-6-cyclopropylpyridine-3-carboxylic acid. To a solution of ethyl 2-cyano-6-cyclopropylpyridine-3-carboxylate (1.8 g, 8.32 mmol) in water (3 mL) and THE (15 mL) was added LiOH·H₂O (699 mg, 16.7 mmol) at 25° C. and stirred for 2 h. The reaction mixture was concentrated under reduced pressure to remove THF, diluted with H₂O (10 mL) and the resulting mixture was lyophilized to give the title compound as a white solid. Y=100%, Li salt. LCMS (ESI): m/z: [M+H]⁺=189.1.

Step 6. 6-Cyclopropyl-2-propanoylpyridine-3-carboxylic acid. To a solution of 2-cyano-6-cyclopropylpyridine-3-carboxylic acid (0.5 g, 2.56 mmol) in THE (5 mL) was added ethylmagnesium bromide (3 M in diethyl ether, 1.28 mL, 3.84 mmol) at 0° C. The mixture was stirred at 25° C. for 5 h, quenched with water (5 mL) at 0° C. and extracted with EtOAc (3×10 mL). The combined organic layers were washed with brine (10 mL), dried over Na₂SO₄, filtered and concentrated under reduced pressure. Purification by FCC (SiO₂, 5-50% EtOAc in petroleum ether gave the title compound as a colourless oil. Y=39%.

Step 7. 2-Cyclopropyl-8-ethyl-6H-pyrido[2,3-d]pyridazin-5-one. To a solution of 6-cyclopropyl-2-propanoylpyridine-3-carboxylic acid (0.21 g, 958 μmol) in EtOH (2 mL) at 25° C. was added hydrazine hydrate (950 μL, 19.2 mmol). The mixture was stirred at 80° C. for 12 h. The reaction was filtered and the filter cake dried to give the title compound as a white solid. Y=73%. ¹H NMR (400 MHz, DMSO-d₆) δ 8.38 (d, J=8 Hz, 1H), 7.74 (d, J=8 Hz, 1H), 2.94 (q, J=8 Hz, 2H), 2.40-2.31 (m, 1H), 1.22 (t, J=8 Hz, 3H), 1.17-1.09 (m, 4H).

### Intermediate C21. 2-cyclopropyl-8-methyl-6H-pyrido[2,3-d]pyridazin-5-one

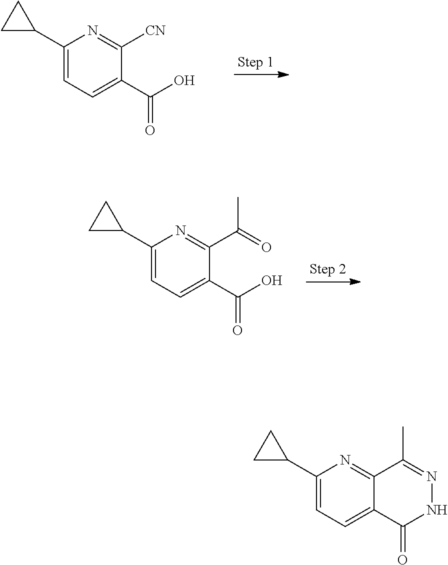

Step 1. 2-Acetyl-6-cyclopropylpyridine-3-carboxylic acid. To a solution of 2-cyano-6-cyclopropylpyridine-3-carboxylic acid (0.5 g, 2.56 mmol) in THE (5 mL) at 0° C. was added ethylmagnesium bromide (3M in diethyl ether, 1.28 mL, 3.84 mmol). The mixture was stirred at 25° C. for 5 h, quenched with water (5 mL) at 0° C., and extracted with EtOAc (3×10 mL). The combined organic layers were washed with brine (10 mL), dried over Na₂SO₄, filtered and concentrated under reduced pressure. Purification by FCC (SiO₂, 5-50% EtOAc in petroleum ether) gave the title compound as a white solid. Y=32%.

Step 2. 2-Cyclopropyl-8-methyl-6H-pyrido[2,3-d]pyridazin-5-one. To a solution of 2-acetyl-6-cyclopropylpyridine-3-carboxylic acid (0.15 g, 731 μmol) in EtOH (1.5 mL) at 25° C. was added hydrazine hydrate (725 μL, 14.6 mmol). The mixture was stirred at 80° C. for 12 h. The reaction was filtered and the filter cake dried to give the title compound as a white solid. Y=82%. ¹H NMR (400 MHz, DMSO-d₆) δ 8.38 (d, J=8 Hz, 1H), 7.73 (d, J=8 Hz, 1H), 2.46 (s, 3H), 2.38-2.33 (m, 1H), 1.17-1.12 (m, 4H).

### Intermediate C21. 2-(2,7-diisopropyl-4-oxo-2,4-dihydro-5H-pyrazolo[3,4-d]pyridazin-5-yl)acetic acid

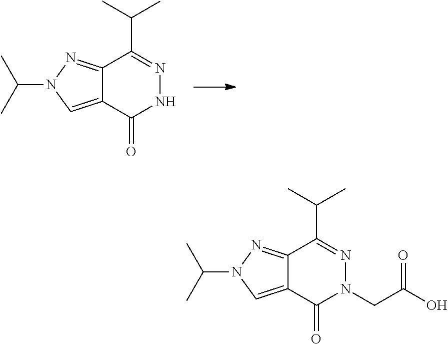

To a solution of 2-bromoacetic acid (39.2 μL, 545 μmol) in THE (2 mL) at 25° C. was added 2,7-diisopropyl-5H-pyrazolo[3,4-d]pyridazin-4-one (Intermediate C12, 100 mg, 454 μmol) and LiO^{t}Bu (164 μL, 1.82 mmol). The RM was stirred at 80° C. for 3 h under N₂, diluted with water (2 mL) and extracted with EtOAc (3×2 mL). 2 M HCl was slowly added dropwise until pH=4-5. The resulting mixture was extracted with EtOAc (3×2 mL). The combined organic layers were washed with brine (3×2 mL), dried over Na₂SO₄, filtered, and concentrated under reduced pressure to give the title compound as a white solid. Y=63%. ¹H NMR (400 MHz, DMSO-d₆) δ 12.85 (br. s, 1H), 8.80 (s, 1H), 4.87-4.76 (m, 1H), 4.69 (s, 2H), 3.35-3.20 (m, 1H), 1.53 (d, J=7 Hz, 6H), 1.32 (d, J=7 Hz, 6H).

### Intermediate C22. 2-(2,7-Diisopropyl-4-oxo-2,4-dihydro-5H-pyrazolo[3,4-d]pyridazin-5-yl)acetic acid

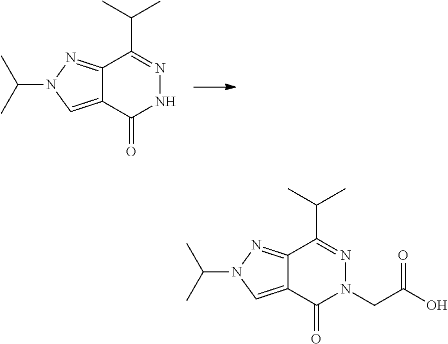

To a solution of 2-bromoacetic acid (39.2 μL, 545 μmol) in THE (2 mL) at 25° C. was added 2,7-diisopropyl-5H-pyrazolo[3,4-d]pyridazin-4-one (Intermediate C12, 100 mg, 454 μmol) and LiO^{t}Bu (145 mg, 1.82 mmol). The reaction mixture was stirred at 80° C. for 3 h under N₂ then diluted with water (2 mL) and extracted with EtOAc (3×2 mL). The mixture was acidified to pH=4˜5 using 2 M HCl_{(aq)}. The resulting mixture was extracted with EtOAc (3×2 mL). The combined organic layers were washed with brine (3×2 mL), dried over Na₂SO₄, filtered, and concentrated under reduced pressure to give the title compound as a white solid. Y=63%. ¹H NMR (400 MHz, DMSO-d₆) δ 12.85 (br. s, 1H), 8.80 (s, 1H), 4.87-4.76 (m, 1H), 4.69 (s, 2H), 3.35-3.20 (m, 1H), 1.53 (d, J=7 Hz, 6H), 1.32 (d, J=7 Hz, 6H).

### Intermediate C23. 2,7-Diisopropyl-2,5-dihydro-4H-pyrazolo[3,4-d]pyridazin-4-one

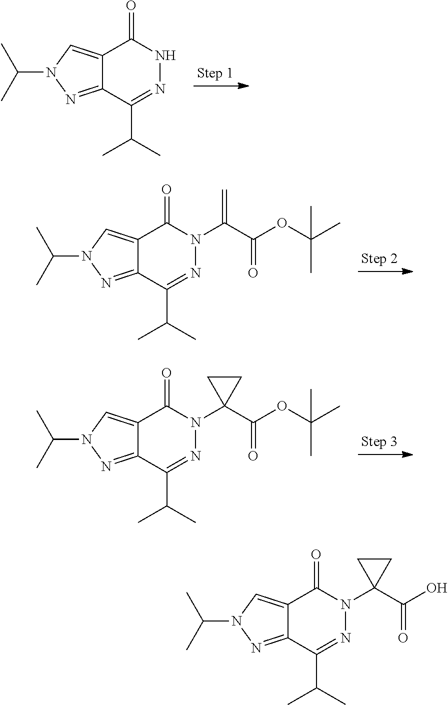

Step 1. Tert-butyl 2-(2,7-diisopropyl-4-oxo-2,4-dihydro-5H-pyrazolo[3,4-d]pyridazin-5-yl)acrylate. To a stirred solution of 2,7-diisopropyl-2,5-dihydro-4H-pyrazolo[3,4-d]pyridazin-4-one (Intermediate C22, 410 mg, 1.86 mmol) in DCM (8 mL) at 0° C. was added PPh₃ (488 mg, 1.86 mmol) then tert-butyl prop-2-ynoate (282 μL, 2.05 mmol). The resulting mixture was stirred at 25° C. for 7 h. The reaction mixture was diluted with H₂O (10 mL) and extracted with DCM (3×10 mL). The combined organic layers were washed with brine (2×10 mL), dried over Na₂SO₄, filtered and concentrated under reduced pressure. The crude material was purified by prep-TLC (SiO₂, Petroleum ether:Ethyl acetate=1:1) to give the title compound as a white solid, Y=85%. ¹H NMR (400 MHz, DMSO-d₆) δ 8.85 (s, 1H), 6.12 (s, 1H), 5.89 (s, 1H), 4.88-4.77 (m, 1H), 3.32-3.26 (m, 1H), 1.53 (d, J=7 Hz, 6H), 1.42 (s, 9H), 1.33 (d, J=7 Hz, 6H).

Step 2. Tert-butyl 1-(2,7-diisopropyl-4-oxo-2,4-dihydro-5H-pyrazolo[3,4-d]pyridazin-5-yl)cyclopropane-1-carboxylate. To a stirred solution of trimethylsulfoxonium iodide (699 mg, 3.18 mmol) in DMSO (6 mL) at 25° C. was added NaH (127 mg, 3.18 mmol, 60% in mineral oil) and the mixture was stirred at 25° C. temperature for 40 min. To the mixture was added a solution of tert-butyl 2-(2,7-diisopropyl-4-oxo-pyrazolo[3,4-d]pyridazin-5-yl)prop-2-enoate (550 mg, 1.59 mmol) in DMSO (5 mL) and the mixture was stirred at 25° C. for 3 h. The reaction mixture was quenched by addition of H₂O (5 mL) at 0° C. The mixture was extracted with EtOAc (3×8 mL) and the combined organic layers were washed with brine (2×8 mL), dried over Na₂SO₄, filtered and concentrated under reduced pressure. The crude material was purified by column chromatography (SiO₂, petroleum ether/ethyl acetate=10/1 to 1/1) to give the title compound as a white solid, Y=49%. ¹H NMR (400 MHz, DMSO-d₆) δ 8.75 (s, 1H), 4.85-4.74 (m, 1H), 3.25-3.27 (m, 1H), 1.66-1.61 (m, 2H), 1.52 (d, J=7 Hz, 6H), 1.41-1.37 (m, 2H), 1.33-1.28 (m, 15H).

Step 3. 1-(2,7-Diisopropyl-4-oxo-2,4-dihydro-5H-pyrazolo[3,4-d]pyridazin-5-yl)cyclopropane-1-carboxylic acid. A mixture of tert-butyl 1-(2,7-diisopropyl-4-oxo-pyrazolo[3,4-d]pyridazin-5-yl)cyclopropane carboxylate (280 mg, 776 mol) and a solution of HCl in EtOAc (3 mL, 4M) was stirred at 20° C. for 2 h. The reaction mixture was filtered and the filter cake was dried under reduced pressure to give the title compound as a white solid, Y=63%. ¹H NMR (400 MHz, DMSO-d₆) δ 12.42 (br. s, 1H), 8.76 (s, 1H), 4.84-4.75 (m, 1H), 3.27-3.23 (m, 1H), 1.66-1.65 (m, 2H), 1.52 (d, J=7 Hz, 6H), 1.44-1.38 (m, 2H), 1.31 (d, J=7 Hz, 6H).

### Intermediate C24. 2-(2-Ethyl-7-(fluoromethyl)-4-oxo-2,4-dihydro-5H-pyrazolo[3,4-d]pyridazin-5-yl)-N-(pyrimidin-2-yl)acetamide

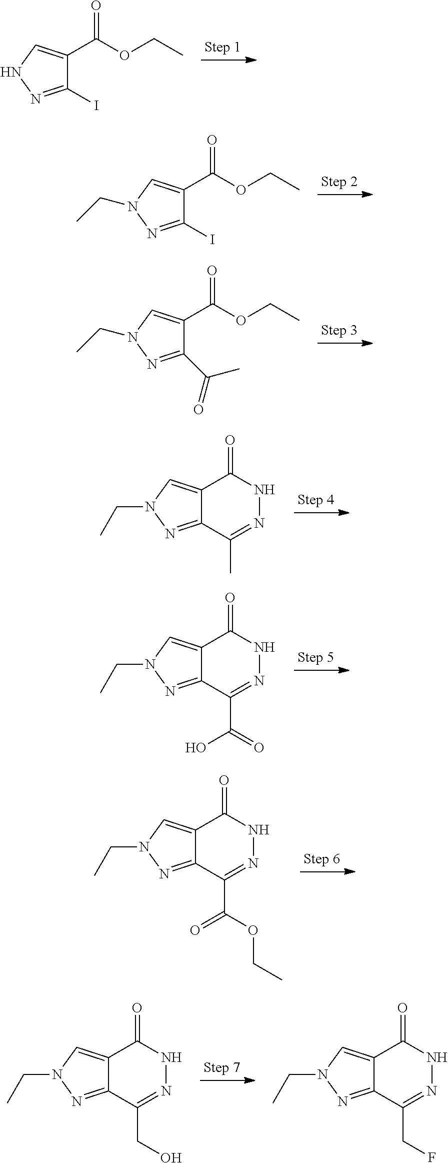

Step 1. Ethyl 1-ethyl-3-iodo-1H-pyrazole-4-carboxylate. To a stirred solution of ethyl 3-iodo-1H-pyrazole-4-carboxylate (5.5 g, 20.7 mmol) in DMF (80 mL) was added EtI (1.82 mL, 22.7 mmol) and Cs₂CO₃ (13.5 g, 41.4 mmol) at 25° C. and the mixture was stirred for 2 hr. The reaction mixture was diluted with H₂O (60 mL) and extracted with ethyl acetate (3×60 mL). The combined organic layers were washed with brine (60 mL), dried over anhydrous Na₂SO₄, filtered and concentrated under reduced pressure. The crude material was purified by column chromatography (SiO₂, Petroleum ether:Ethyl acetate=10:1 to 3:1) to give the title compound as a colorless oil, Y=61%. ¹H NMR (400 MHz, DMSO-d₆) δ 8.30 (s, 1H), 4.25-4.13 (m, 4H), 1.36 (t, J=7 Hz, 3H), 1.27 (t, J=7 Hz, 3H).

Step 2. Ethyl 3-acetyl-1-ethyl-1H-pyrazole-4-carboxylate. To a solution of ethyl 1-ethyl-3-iodo-pyrazole-4-carboxylate (3.70 g, 12.6 mmol) in toluene (30 mL) was added tributyl(1-ethoxyvinyl)stannane (6.37 mL, 18.9 mmol) and Pd(PPh₃)₂Cl₂ (883 mg, 1.26 mmol) at 25° C., the mixture was stirred at 100° C. for 10 hr under N₂. The reaction mixture was concentrated under vacuum and to the crude mixture was added THE (20 mL) and 2N HCl_{(aq)} (20 mL), the resulting mixture was stirred at 25° C. for 2 hr. The mixture was diluted with saturated KF aqueous solution (20 mL) and extracted with EtOAc (3×30 mL). The combined organic layers were washed with brine (2×20 mL), dried over anhydrous Na₂SO₄, filtered and concentrated under vacuum. The crude material was purified by column chromatography (SiO₂, Petroleum ether:Ethyl acetate=10:1 to 2:1) to give the title compound as a colourless oil, Y=60%. ¹H NMR (400 MHz, MeOD-d₄) δ 8.18 (s, 1H), 4.31-4.21 (m, 4H), 2.57 (s, 3H), 1.50 (t, J=7 Hz, 3H), 1.32 (t, J=7 Hz, 3H).

Step 3. 2-Ethyl-7-methyl-2,5-dihydro-4H-pyrazolo[3,4-d]pyridazin-4-one. To a solution of ethyl 3-acetyl-1-ethyl-pyrazole-4-carboxylate (1.4 g, 6.66 mmol) in EtOH (20 mL) was added N₂H₄·H₂O (8.26 mL, 166 mmol) at 25° C. The mixture was stirred at 80° C. for 2 h. The reaction mixture was filtered and the filter cake was dried to give the title compound as a white solid, Y=quantitative. Material was used crude in Step 4.

Step 4. 2-Ethyl-4-oxo-4,5-dihydro-2H-pyrazolo[3,4-d]pyridazine-7-carboxylic acid. To a solution of 2-ethyl-7-methyl-5H-pyrazolo[3,4-d]pyridazin-4-one (150 mg, 842 mol) in H₂O (2 mL) was added KMnO₄ (399 mg, 2.53 mmol) and K₂CO₃ (70 mg, 505 μmol) at 25° C. then the mixture was stirred at 80° C. for 12 h. The reaction was performed twice more in parallel using the exact conditions outlined. The reaction mixture was quenched by addition of saturated Na₂SO_{3(aq)} solution (5 mL), and then resulting mixture was adjusted to pH=5 by addition of 2 M HCl, filtered and the filtrate was extracted with EtOAc (3×5 mL). The combined organic phases were washed with brine (5 mL), dried over anhydrous Na₂SO₄, filtered and concentrated under reduced pressure. The residue was purified by prep-HPLC (column: Waters Xbridge BEH C18 100×30 mm, 10 m; mobile phase: [(A) water (NH₄HCO₃)−(B) ACN]; B: 1-20%, 8 min) and lyophilised to give the title compound as a white solid, Y=38%.

Step 5. Ethyl 2-ethyl-4-oxo-4,5-dihydro-2H-pyrazolo[3,4-d]pyridazine-7-carboxylate. To a solution of 2-ethyl-4-oxo-5H-pyrazolo [3,4-d]pyridazine-7-carboxylic acid (200 mg, 961 mol) in EtOH (5 mL) was added SOCl₂ (697 μL, 9.61 mmol) at 25° C., the mixture was stirred at 50° C. for 2 h. The reaction mixture was adjusted to pH 7˜8 by addition of saturated NaHCO₃ aqueous solution, and the mixture was extracted with EtOAc (3×3 mL). The combined organic layers were washed with brine (1 mL), dried over anhydrous Na₂SO₄, filtered and concentrated under reduced pressure to give the title compound as a white solid, Y=66%.

Step 6. 2-Ethyl-7-(hydroxymethyl)-2,5-dihydro-4H-pyrazolo[3,4-d]pyridazin-4-one. To a solution of ethyl 2-ethyl-4-oxo-5H-pyrazolo[3,4-d]pyridazine-7-carboxylate (150 mg, 635 μmol) in EtOH (4 mL) and THE (1 mL) was added LiCl (108 mg, 2.54 mmol) at 25° C., and the mixture was stirred for 30 min. To the mixture was added NaBH₄ (96 mg, 2.54 mmol) at 0° C. then the mixture was allowed to warm to 25° C. and was stirred for 12 h. The reaction mixture was quenched by addition of H₂O (2 mL) at 0° C. and extracted with EtOAc (3×2 mL). The combined organic layers were washed with brine (2 mL), dried over anhydrous Na₂SO₄, filtered and concentrated under reduced pressure. The crude material was purified by prep-HPLC (column: Waters Xbridge BEH C18 100×30 mm, 10 μm; mobile phase: [(A) water (NH₄HCO₃)−(B) ACN]; B: 1-15%, 10 min) and lyophilised to give the title compound as a white solid, Y=36%.

Step 7. 2-Ethyl-7-(fluoromethyl)-2,5-dihydro-4H-pyrazolo[3,4-d]pyridazin-4-one. To a solution of 2-ethyl-7-(hydroxymethyl)-5H-pyrazolo[3,4-d]pyridazin-4-one (65 mg, 335 mol) in DCM (1 mL) was added BAST (1.00 mmol, 220 μL) at 0° C., the mixture was stirred at 25° C. for 2 hr. The reaction mixture was quenched by addition of H₂O (1 mL) at 0° C., and the resulting mixture was extracted with DCM (3×1 mL). The combined organic layers were washed with brine (1 mL), dried over anhydrous Na₂SO₄, filtered and concentrated under reduced pressure. The residue was purified by prep-HPLC (column: Waters Xbridge BEH C18 100×30 mm, 10 m; mobile phase: [(A) water (NH₄HCO₃)−(B) ACN]; B: 1-35%, 10 min) and lyophilised to give the title compound as a white solid, Y=38%. ¹H NMR (400 MHz, MeOD-d₄) δ 8.57 (s, 1H), 5.59 (s, 1H), 5.47 (s, 1H), 4.51 (q, J=7 Hz, 2H), 1.59 (t, J=7 Hz, 3H).

### Intermediate C25. 2-Ethyl-4-isopropylfuro[2,3-d]pyridazin-7(6H)-one

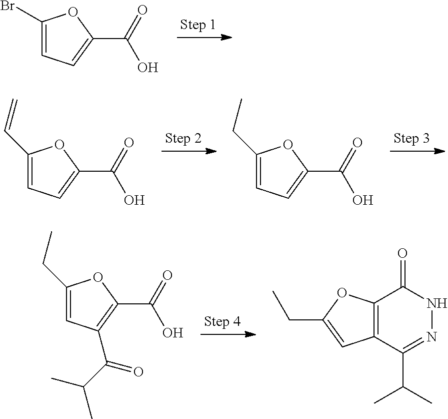

Step 1. 5-Vinylfuran-2-carboxylic acid. To a solution of 5-bromofuran-2-carboxylic acid (4 g, 20.94 mmol) in dioxane (100 mL) and H₂O (20 mL) at 25° C. under N₂ was added 4,4,5,5-tetramethyl-2-vinyl-1,3,2-dioxaborolane (5.33 mL 31.4 mmol), Na₂CO₃ (4.44 g, 41.9 mmol) and Pd(dppf)Cl₂ (3.07 g, 4.19 mmol) and the mixture was stirred at 80° C. for 18 h. The mixture was allowed to cool to room temperature, diluted with H₂O (40 mL) and extracted with EtOAc (3×40 mL). The combined organic layers were washed with brine (40 mL), dried over Na₂SO₄, filtered and the filtrate was concentrated under reduced pressure to give the title compound as a brown oil, Y=99%.

Step 2. 5-Ethylfuran-2-carboxylic acid. To a solution of 5-vinylfuran-2-carboxylic acid (2.85 g, 20.6 mmol) in MeOH (40 mL) was added palladium on carbon (900 mg, 10% wt, 50% in H₂O) under N₂. The suspension was degassed under vacuum and backfilled with H₂ three times. The mixture was stirred under H₂ (15 psi) at 25° C. for 1.5 h. The reaction mixture was filtered through a pad of Celite, the filtrate was concentrated under reduced pressure to give the title compound as a white solid, Y=45%. ¹H NMR (400 MHz, DMSO-d₆) δ 12.81 (s, 1H), 7.11 (d, J=3 Hz, 1H), 6.29 (d, J=3 Hz, 1H), 2.65 (q, J=8 Hz, 2H), 1.18 (t, J=8 Hz, 3H).

Step 3. 5-Ethyl-3-isobutyrylfuran-2-carboxylic acid. To a solution of 5-ethylfuran-2-carboxylic acid (300 mg, 2.14 mmol) in THE (6 mL) was added n-BuLi (2.5 M in THF, 1.71 mL, 4.28 mmol) at −78° C. under N₂, then to above mixture was added N-methoxy-N,2-dimethyl-propanamide (842 mg, 6.42 mmol) at −78° C. and the mixture was stirred at −78° C. for 3.5 h. The reaction mixture was quenched saturated aqueous NH₄Cl (2 mL) at 0° C., then the resulting mixture was adjusted to pH=2 by addition of 1 M HCl_{(aq)} and extracted with EtOAc (3×5 mL). The combined organic layers were dried over Na₂SO₄, filtered and concentrated under reduced pressure. The crude material was purified by prep-TLC (SiO₂, Petroleum ether:Ethyl acetate=0:1) to give the title compound as a white solid, Y=78%.

Step 4. 2-Ethyl-4-isopropylfuro[2,3-d]pyridazin-7(6H)-one. To a solution of 5-ethyl-3-(2-methylpropanoyl)furan-2-carboxylic acid (300 mg, 1.43 mmol) in EtOH (3 mL) under N₂ was added N₂H₄·H₂O (1.49 mL, 30.0 mmol) at 25° C., then the mixture was stirred at 80° C. for 20 h. The reaction mixture was concentrated under reduced pressure to remove EtOH (15 mL), the afforded precipitate was collected and dried under vacuum to give the title compound as a white solid, Y=34%. ¹H NMR (400 MHz, DMSO-d₆) δ 6.84 (s, 1H), 6.68 (br. s, 1H), 3.19-3.10 (m, 1H), 2.89-2.80 (m, 2H), 1.31-1.20 (m, 9H).

### Intermediate C26. 2,7-Diethylfuro[2,3-d]pyridazin-4(5H)-one

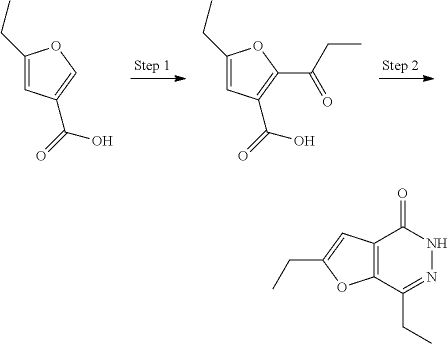

Step 1. 5-Ethyl-2-propionylfuran-3-carboxylic acid. To a solution of 5-ethylfuran-3-carboxylic acid (100 mg, 714 mol) in THE (3 mL) was added dropwise LDA (2 M in THF, 1.25 mL, 2.5 mmol) at −78° C. under N₂ and was stirred for 0.5 h. Then was added N-methoxy-N-methyl-propanamide (251 mg, 2.14 mmol) at −78° C. and the reaction mixture was stirred at −78° C. for 5.5 h. The reaction mixture was allowed to warm to 0° C. and was quenched by addition of H₂O (2 mL). The reaction mixture was extracted with EtOAc (3×2 mL) and the combined organic layers were washed with brine (2 mL), dried over anhydrous Na₂SO₄, filtered and concentrated under reduced pressure. The crude material was purified by prep-TLC (SiO₂, Petroleum ether:Ethyl acetate=1:1) to give the title compound as a white solid, Y=71%. ¹H NMR (400 MHz, DMSO-d₆) δ 13.53 (s, 1H), 6.65 (s, 1H), 2.98 (q, J=7 Hz, 2H), 2.72 (q, J=8 Hz, 2H), 1.22 (t, J=8 Hz, 3H), 1.07 (t, J=7 Hz, 3H).

Step 2. 2,7-Diethylfuro[2,3-d]pyridazin-4(5H)-one. To a solution of 5-ethyl-2-propanoyl-furan-3-carboxylic acid (120 mg, 611 mol) in EtOH (1 mL) was added N₂H₄·H₂O (12.8 mmol, 637 μL) at 25° C., then the mixture was stirred at 80° C. for 21 h under N₂. The mixture was filtered, and the filter cake was dried under vacuum to give the title compound as a white solid, Y=99%. ¹H NMR (400 MHz, DMSO-d₆) δ 12.56 (br. s, 1H), 6.75 (s, 1H), 2.87-2.77 (m, 4H), 1.35-1.20 (m, 6H).

### Intermediate C27. 2-Ethyl-7-methylfuro[2,3-d]pyridazin-4(5H)-one

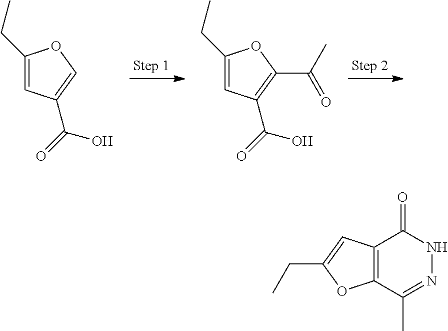

Step 1. 2-Acetyl-5-ethylfuran-3-carboxylic acid. To a solution of 5-ethylfuran-3-carboxylic acid (100 mg, 714 mol) in THE (1 mL) was added LDA (2 M in THF, 1.07 mL, 2.14 mmol) at −78° C. under N₂. The mixture was stirred at for 0.5 h then was added N-methoxy-N-methylacetamide (152 μL, 1.43 mmol) at −78° C. The mixture was stirred at −78° C. for 1 h. The reaction was quenched by addition of saturated NH₄Cl aqueous solution (2 mL), and the resulting mixture was adjusted to pH=3 by aqueous 1 M HCl (3 mL) and extracted with ethyl acetate (3×2 mL). The combined organic layers were washed with brine (3 mL), dried over Na₂SO₄, filtered and concentrated under reduced pressure to give the title compound as a yellow gum, Y=77%.

Step 2. 2-Ethyl-7-methylfuro[2,3-d]pyridazin-4(5H)-one. To a solution of 2-acetyl-5-ethyl-furan-3-carboxylic acid (100 mg, 549 mol) in EtOH (2 mL) was added N₂H₄·H₂O (1.0 mmol, 544 μL) at 25° C., then the mixture was stirred at 70° C. for 4 h under N₂ atmosphere. The reaction mixture was concentrated under reduced pressure to give the title compound as a white solid, Y=80 mg, used as is.

### Intermediate C28. 7-Isopropylfuro[2,3-d]pyridazin-4(5H)-one

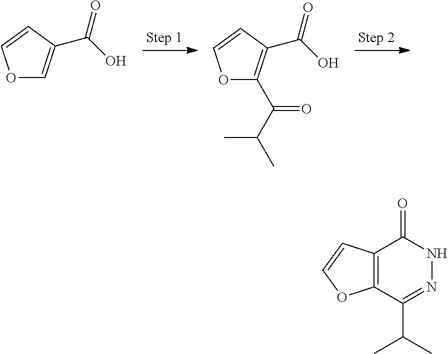

Step 1. 2-Isobutyrylfuran-3-carboxylic acid. To a solution of furan-3-carboxylic acid (3.0 g, 26.8 mmol) in THE (50 mL) at −78° C. under N₂ was added LDA (2 M in THF, 26.8 mL, 53.6 mmol) and the solution was stirred for 0.5 h. To the mixture was added N-methoxy-N,2-dimethyl-propanamide (5.27 g, 40.2 mmol) at −78° C. and the mixture was stirred at −78° C. for 2 h. The mixture was quenched by saturated aqueous NH₄Cl (30 mL) and the resulting mixture was extracted with EtOAc (5×30 mL). The combined organic layers were washed with brine (3×30 mL), dried over anhydrous Na₂SO₄, filtered and concentrated under reduced pressure. The crude product was purified by column chromatography (SiO₂, ethyl acetate) to give the title compound as a white solid, Y=92%. ¹H NMR (400 MHz, DMSO-d₆) δ 7.79 (d, J=8 Hz, 1H), 7.51 (d, J=8 Hz, 1H), 3.65-3.49 (m, 1H), 1.65-0.96 (m, 6H).

Step 2. 7-Isopropylfuro[2,3-d]pyridazin-4(5H)-one. To a solution of 2-(2-methylpropanoyl)furan-3-carboxylic acid (2.0 g, 11.0 mmol) in n-BuOH (30 mL) was added N₂H₄·H₂O (10.9 mL, 222 mmol) and TsOH (3.78 g, 22.0 mmol) at 25° C., the mixture was stirred at reflux for 12 h. The reaction mixture was diluted with H₂O (20 mL) and the resulting mixture was extracted with ethyl acetate (3×20 mL). The combined organic layers were washed with brine (20 mL), dried over anhydrous Na₂SO₄, filtered and concentrated under reduced pressure to give the title compound as yellow solid.

### Intermediate C29. 2-Cyclopropyl-7-isopropylfuro[2,3-d]pyridazin-4(5H)-one

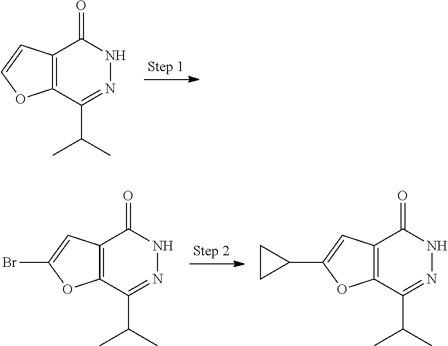

Step 1. 2-Bromo-7-isopropylfuro[2,3-d]pyridazin-4(5H)-one. To a solution of 7-isopropyl-5H-furo[2,3-d]pyridazin-4-one (Intermediate C28, 800 mg, 4.49 mmol) in THE (15 mL) at −78° C. under N₂ was added n-BuLi (2.5 Min THF, 3.59 mL, 8.98 mmol) and the mixture was stirred at −78° C. for 0.5 h. Then 1,2-dibromo-1,1,2,2-tetrachloro-ethane (647 μL, 5.39 mmol) was added at −78° C. and the mixture was stirred at 25° C. for 2 h. The reaction mixture was diluted with H₂O (10 mL) and extracted with EtOAc (3×20 mL). The combined organic layers were washed with brine (2×20 mL), dried over anhydrous Na₂SO₄, filtered and concentrated under reduced pressure. The crude material was purified by column chromatography (SiO₂, petroleum ether:ethyl acetate=10:1 to 1:1) to the give title compound as a yellow solid, Y=24%. ¹H NMR (400 MHz, DMSO-d₆) δ 7.32 (s, 1H), 3.27-3.18 (m, 1H), 1.27 (d, J=7 Hz, 6H).

Step 2. 2-Cyclopropyl-7-isopropylfuro[2,3-d]pyridazin-4(5H)-one. To a solution of 2-bromo-7-isopropyl-5H-furo[2,3-d]pyridazin-4-one (280 mg, 1.09 mmol) in toluene (5 mL) and H₂O (0.5 mL) at 25° C. under N₂ were added cyclopropylboronic acid (140 mg, 1.63 mmol), Pd(OAc)₂ (12 mg, 55 mol), tricyclohexylphosphine (31 mg, 109 mol) and K₃PO₄ (809 mg, 3.81 mmol) and the mixture was stirred at 80° C. for 4 h. The mixture was cooled to 25° C., diluted with H₂O (5 mL) and extracted with ethyl acetate (3×5 mL). The combined organic layers were washed with brine (5 mL), dried over anhydrous Na₂SO₄, filtered and concentrated under reduced pressure. The crude material was purified by column chromatography (SiO₂, petroleum ether:ethyl acetate=10:1 to 2:1) to give the title compound as a yellow solid, Y=21%.

### Intermediate C30. 2-Chloro-7-isopropylfuro[2,3-d]pyridazin-4(5H)-one

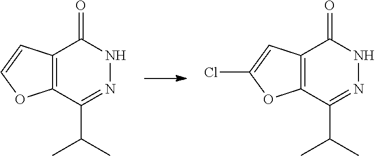

To a solution of 7-isopropyl-5H-furo[2,3-d]pyridazin-4-one (Intermediate C28, 200 mg, 1.12 mmol) in DMF (3 mL) was added NCS (300 mg, 2.25 mmol) at 25° C., the solution was stirred for 2 h under N₂. The reaction mixture was quenched by addition of saturated Na₂S₂O_{3(aq)} (3 mL) at 0° C. and then extracted with ethyl acetate (3×5 mL). The combined organic layers were washed with brine (5 mL), dried over Na₂SO₄, filtered and concentrated under reduced pressure. The crude material was purified by prep-TLC (SiO₂, petroleum ether:ethyl acetate=1:1) to give the title compound as a yellow solid, Y=15%. ¹H NMR (400 MHz, DMSO-d₆) δ 12.80 (s, 1H), 7.24 (s, 1H), 3.30-3.20 (m, 1H), 1.28 (d, J=7 Hz, 6H).

### Intermediate C31. 2-Chloro-7-isopropylthieno[2,3-d]pyridazin-4(5H)-one

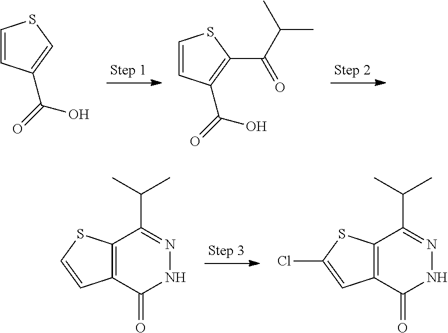

Step 1. 2-Isobutyrylthiophene-3-carboxylic acid. To a stirred solution of thiophene-3-carboxylic acid (1.0 g, 7.80 mmol) in THE (10 mL) was added LDA (2 M, 8.59 mL, 17.2 mmol) dropwise at 0° C. under N₂, following the addition the mixture was stirred at 0° C. for 15 min. A solution of N-methoxy-N,2-dimethylpropanamide (1.13 g, 8.58 mmol) in THE (5 mL) was added dropwise to the mixture at 0° C., then the reaction mixture was allowed to warm to 25° C. then was stirred for a further 2 h. The reaction mixture was adjusted to pH=4 by addition of 2 N HCl_{(aq)} solution, the resulting mixture was extracted with EtOAc (3×10 mL). The combined organic phase was washed with brine (5 mL), dried with anhydrous Na₂SO₄, filtered and concentrated in vacuum to give the title compound as a solid, used as is. ¹H NMR (400 MHz, DMSO-d₆) δ 13.14 (br., 1H), 7.84 (d, J=5 Hz, 1H), 7.37 (d, J=5 Hz, 1H), 3.35-3.25 (m, 1H), 1.10 (d, J=7 Hz, 6H).

Step 2. 7-Isopropylthieno[2,3-d]pyridazin-4(5H)-one. To a solution of 2-(2-methylpropanoyl)thiophene-3-carboxylic acid (650 mg, 3.28 mmol) in EtOH (8 mL) was added hydrazine hydrate (32.8 mmol, 1.63 mL) and the mixture was stirred at 80° C. for 1 h. The mixture was concentrated under reduced pressure to give the title compound as a white solid, Y=71%.

Step 3. 2-Chloro-7-isopropylthieno[2,3-d]pyridazin-4(5H)-one. To a solution of 7-isopropyl-5H-thieno[2,3-d]pyridazin-4-one (90 mg, 463 mol) in DMF (1 mL) was added NCS (124 mg, 927 mol) at 25° C. and the mixture was stirred for 1 h. Saturated Na₂S₂O_{3(aq)} solution (2 mL) was added, and the resulting mixture was stirred at 0° C. for 10 min and then extracted with ethyl acetate (3×2 mL). The combined organic phases were washed with brine (2 mL), dried over anhydrous Na₂SO₄, filtered and the filtrate was concentrated under vacuum. The crude material was purified by prep-TLC (SiO₂, ethyl acetate) to give the title compound as a white solid, Y=66%.

### Intermediate C32. 2-Bromo-7-isopropylfuro[2,3-d]pyridazin-4(5H)-one

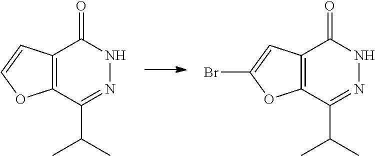

To a solution of 7-isopropyl-5H-furo[2,3-d]pyridazin-4-one (Intermediate C28, 120 mg, 673 μmol) in DMF (2 mL) at 25° C. under N₂ was added NBS (240 mg, 1.35 mmol) and the mixture was stirred for 3 h. The reaction mixture was quenched by addition of saturated Na₂S₂O_{3(aq)} solution (2 mL) and extracted with ethyl acetate (3×0.5 mL). The combined organic layers were washed with brine (2×0.5 mL), dried over Na₂SO₄, filtered and concentrated under reduced pressure. The residue was purified by prep-TLC (SiO₂, petroleum ether:ethyl acetate=1:1) to give the title compound as a white solid, Y=35%. ¹H NMR (DMSO-d₆) δ 12.77 (s, 1H), 7.31 (s, 1H), 3.30-3.20 (m, 1H), 1.28 (d, J=7 Hz, 6H).

### Intermediate C33. 4-Isopropylthieno[2,3-d]pyridazin-7(6H)-one

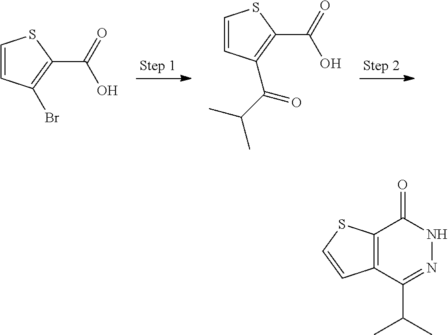

Step 1. 3-Isobutyrylthiophene-2-carboxylic acid. To a stirred solution of 3-bromothiophene-2-carboxylic acid (2.0 g, 9.66 mmol) in THE (20 mL) at −78° C. under N₂ was added n-BuLi (2.5 M, 7.73 mL, 19.3 mmol) and the mixture was stirred at −78° C. for 30 min. A solution of N-methoxy-N, 2-dimethyl-propanamide (1.52 g, 11.6 mmol) in THE (3 mL) was added at −78° C. and the mixture was allowed to warm to 25° C., then stirred for 1 h. The mixture was adjusted to pH=4-5 by addition of 2 M HCl (10 mL), and the resulting mixture was diluted with H₂O (5 mL) and extracted with ethyl acetate (3×20 mL). The combined organic layers were washed with brine (30 mL), dried over anhydrous Na₂SO₄, filtered and concentrated under reduced pressure. The residue was purified by column chromatography (SiO₂, petroleum ether:ethyl acetate=10:1 to 3:1) to give the title compound as a white solid, Y=52%.

Step 2. 4-Isopropylthieno[2,3-d]pyridazin-7(6H)-one. To a solution of 3-(2-methylpropanoyl) thiophene-2-carboxylic acid (0.50 g, 2.52 mmol) in EtOH (5 mL) was added N₂H₄·H₂O (50.4 mmol, 2.50 mL) at 25° C., the mixture was stirred at 80° C. for 2 h. The solution was partially concentrated in vacuum, then filtered and the filter cake was dried to give the title compound as a white solid. Y=71%.

### Intermediate C34. 2-Bromo-4-isopropylthieno[2,3-d]pyridazin-7(6H)-one

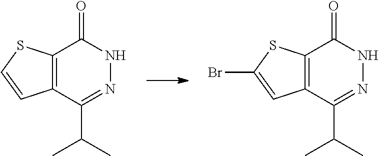

To a stirred solution of 4-isopropyl-6H-thieno[2,3-d]pyridazin-7-one (Intermediate C33, 1.3 g, 6.69 mmol) in THE (15 mL) was added LDA (2 M in THF, 8.37 mL, 16.7 mmol) at −78° C. under nitrogen and the mixture was stirred for 0.5 h. To the reaction mixture was added 1,2-dibromo-1,1,2,2-tetrachloro-ethane (965 μL, 8.03 mmol) at −78° C. and the reaction was allowed to warm to 25° C. then stirred for a further 2 h. The reaction mixture was quenched by addition of H₂O (15 mL) at 0° C. and then extracted with EtOAc (3×25 mL). The combined organic layers were washed with brine (8 mL), dried over Na₂SO₄, filtered and concentrated under reduced pressure. The crude material was purified by column chromatography (SiO₂, Petroleum ether:Ethyl acetate=2:1 to 0:1) to give the title as a yellow solid, Y=82%.

### Intermediate C35. 1-(2-Cyclopropyl-7-isopropyl-4-oxo-2,4-dihydro-5H-pyrazolo[3,4-d]pyridazin-5-yl)cyclopropane-1-carboxylic acid

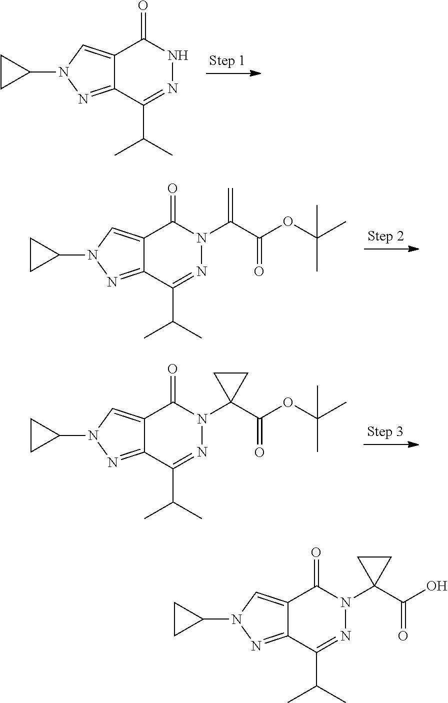

Step 1. Tert-butyl 2-(2-cyclopropyl-7-isopropyl-4-oxo-2,4-dihydro-5H-pyrazolo[3,4-d]pyridazin-5-yl)acrylate. To a solution of 2-cyclopropyl-7-isopropyl-5H-pyrazolo[3,4-d]pyridazin-4-one (Intermediate C13, 1.1 g, 5.04 mmol) in DCM (10 mL) was added PPh₃ (661 mg, 2.52 mmol) at 0° C. and the mixture was stirred for 30 min. Then was added tert-butyl prop-2-ynoate (954 mg, 7.56 mmol, 1.04 mL) and the mixture was stirred at 25° C. for 2 h. The reaction mixture was quenched by addition of H₂O (15 mL) and extracted with EtOAc (3×15 mL). The combined organic layers were washed with brine (15 mL), dried over Na₂SO₄, filtered and concentrated under reduced pressure. The residue was purified by prep-TLC (SiO₂, Petroleum ether:Ethyl acetate=1:1) to give the title compound as a white solid, Y=69%.

Step 2. Tert-butyl 1-(2-cyclopropyl-7-isopropyl-4-oxo-2,4-dihydro-5H-pyrazolo[3,4-d]pyridazin-5-yl)cyclopropane-1-carboxylate. To a solution of trimethylsulfoxonium iodide (639 mg, 2.90 mmol) in DMSO (5 mL) was added NaH (116 mg, 2.90 mmol, 60% in mineral oil) at 25° C. and the mixture was stirred for 30 min. To the mixture was added a solution of tert-butyl 2-(2-cyclopropyl-7-isopropyl-4-oxo-pyrazolo[3,4-d]pyridazin-5-yl)prop-2-enoate (500 mg, 1.45 mmol) in DMSO (5 mL) at 25° C. and the mixture was stirred for 2 h. The reaction mixture was diluted with H₂O (5 mL) and extracted with EtOAc (3×5 mL). The combined organic layers were washed with brine (5 mL), dried over Na₂SO₄, filtered and concentrated under reduced pressure. The crude material was purified by prep-HPLC (column: Phenomenex Luna C18 150×30 mm, 5 m; mobile phase: [(A) water (TFA)−(B) ACN]; B: 55-85%, 8 min) and lyophilised to give the title compound as a white solid, Y=38%.

Step 3. 1-(2-Cyclopropyl-7-isopropyl-4-oxo-2,4-dihydro-5H-pyrazolo[3,4-d]pyridazin-5-yl)cyclopropane-1-carboxylic acid. A mixture of tert-butyl 1-(2-cyclopropyl-7-isopropyl-4-oxo-pyrazolo[3,4-d]pyridazin-5-yl)cyclopropanecarboxylate (200 mg, 558 mol) and 4 M HCl in EtOAc (2.79 mL) was stirred at 25° C. for 2 h. The reaction mixture was concentrated under reduced pressure to give the title compound as a yellow solid, Y=89%.

### Intermediate C36. 2-Cyclobutyl-7-isopropyl-2,5-dihydro-4H-pyrazolo[3,4-d]pyridazin-4-one

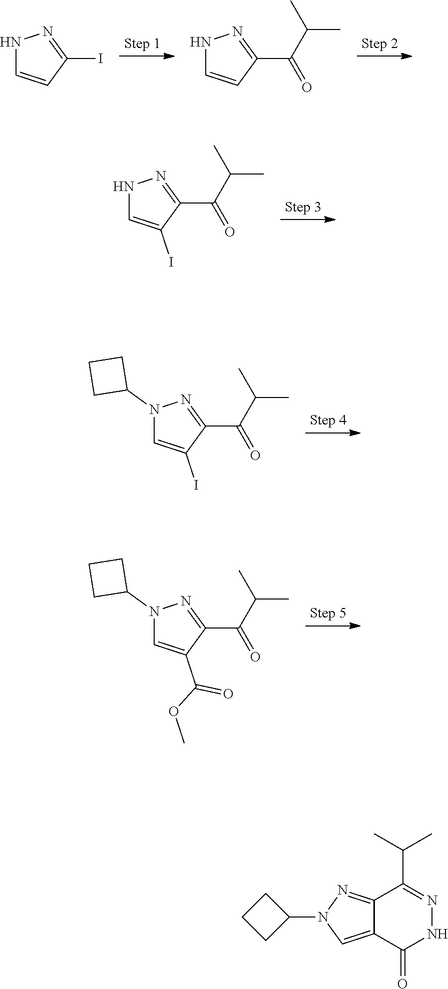

Step 1. 2-Methyl-1-(1H-pyrazol-3-yl)propan-1-one. To a mixture of i-PrMgCl·LiCl complex solution in THE (1.3 M, 59.5 mL, 77.4 mmol) and THE (100 mL) at 0° C. under N₂ was added N-methoxy-N,2-dimethyl-propanamide (5.07 g, 38.7 mmol). The mixture was stirred at 0° C. for 1 h then treated with 3-iodo-1H-pyrazole (5.0 g, 25.8 mmol). The mixture was allowed to warm to 25° C. then was stirred for a further 3 h. The reaction mixture was quenched by addition of saturated NH₄Cl_{(aq)} (100 mL) at 25° C. and extracted with ethyl acetate (3×50 mL). The combined organic layers were washed with brine (3×50 mL), dried over Na₂SO₄, filtered and concentrated under reduced pressure to give the title compound as a yellow oil.

Step 2. 1-(4-Iodo-1H-pyrazol-3-yl)-2-methylpropan-1-one. To a solution of 2-methyl-1-(1H-pyrazol-3-yl)propan-1-one (5.0 g, 36.2 mmol) in AcOH (60 mL) at 25° C. under N₂ was added NIS (12.2 g, 54.3 mmol). The mixture was stirred for 8 h then diluted with H₂O (60 mL) and extracted with ethyl acetate (3×30 mL). The combined organic layers were washed with brine (30 mL), dried over Na₂SO₄, filtered and concentrated under reduced pressure. The crude material was purified by column chromatography (SiO₂, petroleum ether:ethyl acetate=10:1 to 1:1) to give the title compound as a yellow solid, Y=62%. ¹H NMR (400 MHz, DMSO-d₆) δ 13.78 (s, 1H), 8.09 (s, 1H), 3.72-3.60 (m, 1H), 1.08 (d, J=7 Hz, 6H).

Step 3. 1-(1-Cyclobutyl-4-iodo-1H-pyrazol-3-yl)-2-methylpropan-1-one. To a mixture of 1-(4-iodo-1H-pyrazol-3-yl)-2-methyl-propan-1-one (5.9 g, 22.3 mmol) and K₂CO₃ (4.63 g, 33.5 mmol) in DMF (60 mL) at 25° C. was added bromocyclobutane (33.5 mmol, 3.16 mL). The mixture was stirred at 65° C. for 16 h then the reaction mixture was diluted with H₂O (50 mL) and extracted with EtOAc (3×50 mL). The combined organic layers were washed with brine (3×50 mL), dried over anhydrous Na₂SO₄, filtered and concentrated under reduced pressure. The crude material was purified by column chromatography (SiO₂, petroleum ether:ethyl acetate=10:1 to 1:1) to give the title compound as a white solid, Y=51%. ¹H NMR (400 MHz, DMSO-d₆) δ 8.21 (s, 1H), 5.20-4.70 (m, 1H), 3.95-3.50 (m, 1H), 2.47-2.31 (m, 4H), 1.84-1.77 (m, 2H), 1.08 (d, J=7 Hz, 6H).

Step 4. Methyl 1-cyclobutyl-3-isobutyryl-1H-pyrazole-4-carboxylate. To a solution of 1-(1-cyclobutyl-4-iodo-pyrazol-3-yl)-2-methyl-propan-1-one (1.0 g, 3.14 mmol) in MeOH (10 mL) was added TEA (1.31 mL, 9.43 mmol) and Pd(dppf)Cl₂ (230 mg, 314 mol) at 25° C. The mixture was vacuum purged and backfilled with CO three times then stirred at 70° C. for 12 hr under CO. The reaction mixture was filtered and the filtrate was concentrated under reduced pressure. To the crude material was added H₂O (10 mL) and the mixture was extracted with ethyl acetate (3×10 mL). The combined organic layers were washed with brine (2×10 mL), dried over Na₂SO₄, filtered and concentrated under reduced pressure to give the title compound as a solid, used as is.

Step 5. 2-Cyclobutyl-7-isopropyl-2,5-dihydro-4H-pyrazolo[3,4-d]pyridazin-4-one. To a solution of methyl 1-cyclobutyl-3-(2-methylpropanoyl)pyrazole-4-carboxylate (1.6 g, 6.39 mmol) in EtOH (20 mL) at 25° C. under N₂ was added N₂H₄·H₂O (6.34 mL, 128 mmol). The mixture was stirred at 80° C. for 3 h then the reaction mixture was cooled to 25° C. and diluted with 1 M HCl_{(aq)} (25 mL). The resulting mixture was filtered and the filter cake was dried under reduced pressure to give the title compound as a solid, Y=40%. ¹H NMR (400 MHz, DMSO-d₆) δ 11.84 (s, 1H), 8.72 (s, 1H), 7.54-7.39 (m, 1H), 5.20-4.80 (m, 1H), 3.50-3.10 (m, 1H), 2.58-2.44 (m, 4H), 1.85-1.75 (m, 2H), 1.25 (d, J=7 Hz, 6H).

### Intermediate C37. 2-Cyclopropyl-4-isopropylthieno[2,3-d]pyridazin-7(6H)-one

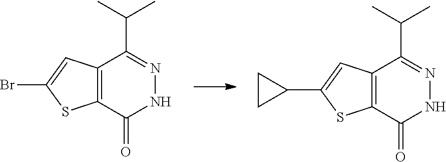 [SMILES: CC(C)c1n[nH]c(=O)c2sc(Br)cc12 ; CC(C)c1n[nH]c(=O)c2sc(C3CC3)cc12]

To a solution of 2-bromo-4-isopropyl-6H-thieno[2,3-d]pyridazin-7-one (Intermediate C34, 600 mg, 2.20 mmol) in toluene (6 mL) and H₂O (0.3 mL) at 25° C. under N₂ was added cyclopropylboronic acid (245 mg, 2.86 mmol), tricyclohexylphosphine (62 mg, 220 mol, 10 mol %), K₃PO₄ (1.63 g, 7.69 mmol) and Pd(OAc)₂ (25 mg, 110 mol). The mixture was stirred at 110° C. for 2 h. The reaction mixture was diluted with H₂O (10 mL) and then extracted with EtOAc (3×10 mL). The combined organic layers were washed with brine (10 mL), dried over anhydrous Na₂SO₄, filtered and concentrated under reduced pressure. The crude material was purified by column chromatography (SiO₂, petroleum ether:ethyl acetate=1:1) to give the title compound as a white solid, Y=39%.

### Intermediate C38. 2-(2-Cyclopropyl-7-isopropyl-4-oxofuro[2,3-d]pyridazin-5(4H)-yl)acetic acid

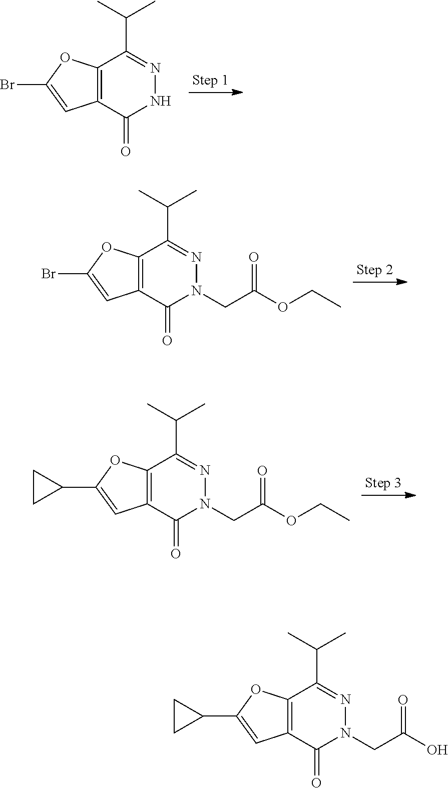

Step 1. Ethyl 2-(2-bromo-7-isopropyl-4-oxofuro[2,3-d]pyridazin-5(4H)-yl)acetate. To a solution of 2-bromo-7-isopropyl-5H-furo[2,3-d]pyridazin-4-one (Intermediate C32,700 mg, 2.72 mmol) in DMF (10 mL) at 25° C. was added K₂CO₃ (1.13 g, 8.17 mmol) and ethyl 2-bromoacetate (361 μL, 3.27 mmol) then the mixture was stirred at 80° C. for 3 h. The reaction mixture was diluted with H₂O (10 mL) and extracted with ethyl acetate (3×10 mL). The combined organic layers were washed with brine (10 mL), dried over anhydrous Na₂SO₄, filtered and concentrated under reduced pressure to give the title compound as a yellow oil. ¹H NMR (400 MHz, DMSO-d₆) δ 7.39 (s, 1H), 4.89 (s, 2H), 4.15 (q, J=7 Hz, 2H), 3.30-3.21 (m, 1H), 1.29 (d, J=7 Hz, 6H), 1.20 (t, J=7 Hz, 3H).

Step 2. Ethyl 2-(2-cyclopropyl-7-isopropyl-4-oxofuro[2,3-d]pyridazin-5(4H)-yl)acetate. To a solution of ethyl 2-(2-bromo-7-isopropyl-4-oxo-furo[2,3-d]pyridazin-5-yl)acetate (1.0 g, 2.91 mmol) in toluene (20 mL) and H₂O (2 mL) at 25° C. was added cyclopropylboronic acid (375 mg, 4.37 mmol), Pd(OAc)₂ (33 mg, 146 mol), P(Cy)₃ (83 mg, 291 μmol) and K₃PO₄ (2.16 g, 10.2 mmol) then the mixture was stirred at 80° C. for 2 h. The reaction mixture was cooled to at 25° C., diluted with H₂O (20 mL) and extracted with ethyl acetate (3×20 mL). The combined organic layers were washed with brine (20 mL), dried over anhydrous Na₂SO₄, filtered and concentrated under reduced pressure. The crude material was purified by column chromatography (SiO₂, petroleum ether:ethyl acetate=10:1 to 1:1) to give the title compound as a yellow gum, Y=68%. ¹H NMR (400 MHz, DMSO-d₆) δ 6.80 (s, 1H), 4.86 (s, 2H), 4.14 (q, J=7 Hz, 2H), 3.27-3.15 (m, 1H), 2.26-2.14 (m, 1H), 1.29 (d, J=7 Hz, 6H), 1.19 (t, J=7 Hz, 3H), 1.11-1.04 (m, 2H), 1.00-0.93 (m, 2H).

Step 3. 2-(2-Cyclopropyl-7-isopropyl-4-oxofuro[2,3-d]pyridazin-5(4H)-yl)acetic acid. To a solution of ethyl 2-(2-cyclopropyl-7-isopropyl-4-oxo-furo[2,3-d]pyridazin-5-yl)acetate (150 mg, 493 mol) in THE (2 mL) and H₂O (0.4 mL) 25° C. was added LiOH·H₂O (62 mg, 1.48 mmol) and the mixture was stirred for 2 h. The reaction mixture was adjusted to pH˜4 with 2 M HCl_{(aq)} and extracted with ethyl acetate (3×3 mL). The combined organic layers were washed with brine (3 mL), dried over anhydrous Na₂SO₄, filtered and concentrated under reduced pressure to give the title compound as a white solid, Y=40%.

### Intermediate C39. 2-(2-(Ethylamino)-4-isopropyl-7-oxothieno[2,3-d]pyridazin-6(7H)-yl)acetic acid

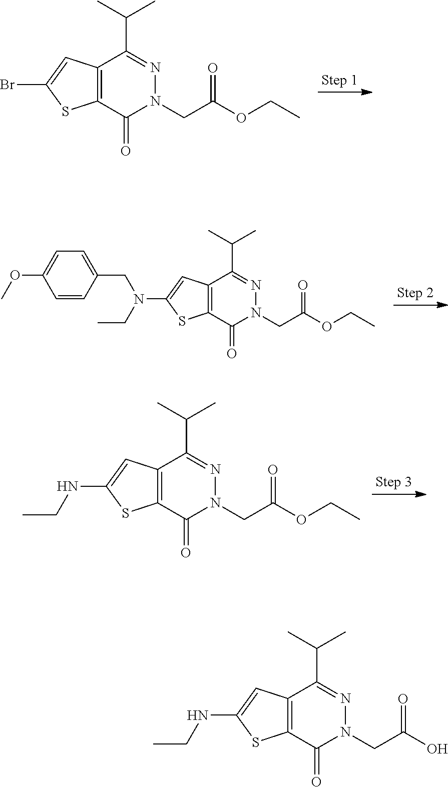

Step 1. Ethyl 2-(2-(ethyl(4-methoxybenzyl)amino)-4-isopropyl-7-oxothieno[2,3-d]pyridazine-6(7H)-yl)acetate. To a solution of ethyl 2-(2-bromo-4-isopropyl-7-oxo-thieno[2,3-d]pyridazine-6-yl)acetate (Intermediate D4, 400 mg, 1.11 mmol) in dioxane (4 mL) at 25° C. was added BINAP (69 mg, 111 mol), N-[(4-methoxyphenyl)methyl]ethanamine (552 mg, 3.34 mmol), Pd(OAc)₂ (25.0 mg, 111 mol) and Cs₂CO₃ (544 mg, 1.67 mmol) then the mixture was stirred at 105° C. for 4 hr. The reaction mixture was filtered, and the filtrate diluted with H₂O (4 mL) and extracted with EtOAc (3×4 mL). The combined organic layers were washed with brine (4 mL), dried over anhydrous Na₂SO₄, filtered and concentrated under reduced pressure. The crude material was purified by column chromatography (SiO₂, Petroleum ether:Ethyl acetate=1:1) to give the title compound as a white solid, Y=81%. ¹H NMR (400 MHz, DMSO-d₆) δ 7.26 (d, J=9 Hz, 2H), 6.92 (d, J=9 Hz, 2H), 6.29 (s, 1H), 4.79 (s, 2H), 4.58 (s, 2H), 4.20-4.10 (m, 2H), 3.73 (s, 3H), 3.55-3.45 (m, 2H), 3.23-3.11 (m, 1H), 1.25-1.11 (m, 12H).

Step 2. Ethyl 2-(2-(ethylamino)-4-isopropyl-7-oxothieno[2,3-d]pyridazin-6(7H)-yl)acetate. A mixture of ethyl 2-[2-[ethyl-[(4-methoxyphenyl)methyl]amino]-4-isopropyl-7-oxo-thieno [2,3-d]pyridazin-6-yl]acetate (200 mg, 451 mol) and TFA (2 mL) was stirred at 70° C. for 12 hr. The reaction mixture was concentrated under reduced pressure to give the title compound as a white solid, Y=86%.

Step 3. 2-(2-(ethylamino)-4-isopropyl-7-oxothieno[2,3-d]pyridazin-6(7H)-yl)acetic acid. To a mixture of ethyl 2-[2-(ethylamino)-4-isopropyl-7-oxo-thieno[2,3-d]pyridazin-6-yl]acetate (250 mg, 773 mol) in THE (1 mL) and H₂O (1 mL) at 25° C. was added LiOH H₂O (65 mg, 1.55 mmol). The mixture was stirred at 25° C. for 2 hr. The reaction mixture was diluted with H₂O (3 mL) and extracted with EtOAc (3×3 mL). Then the aqueous phase was adjusted to pH=4˜5 by aqueous solution 2N HCl and the resulting mixture was extracted with EtOAc (3×3 mL). The combined organic layers were washed with brine (2×3 mL), dried over anhydrous Na₂SO₄, filtered and concentrated under reduced pressure to give the title compound as a white solid, Y=88%. ¹H NMR (400 MHz, DMSO-d₆) δ 12.65 (br., 1H), 7.65 (s, 1H), 6.09 (s, 1H), 4.70 (s, 2H), 3.35-3.02 (m, 3H), 1.24-1.17 (m, 9H).

### Intermediate D1. 2-{2-Cyclopropyl-7-methyl-4-oxo-4H,5H-furo[2,3-d]pyridazin-5-yl}acetic acid

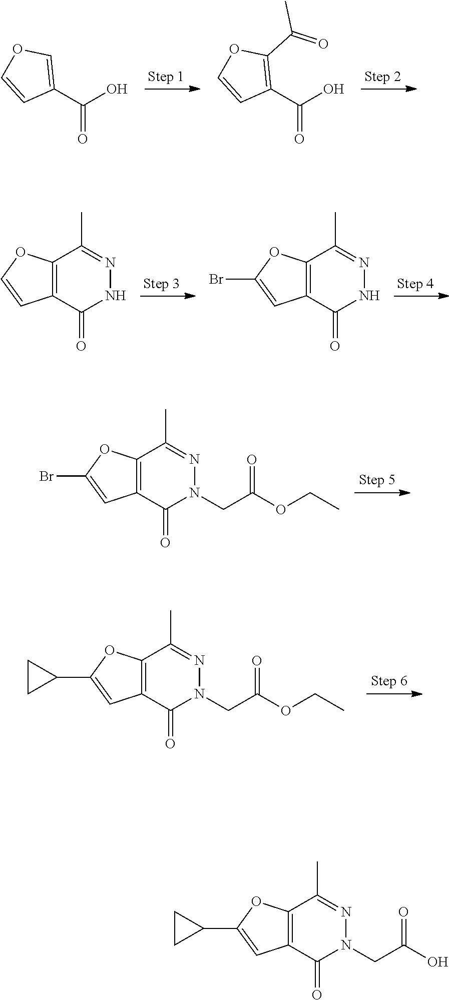

Step 1. 2-Acetylfuran-3-carboxylic acid. To a solution of furan-3-carboxylic acid (25 g, 223 mmol, 1 eq) in THE (200 mL) was added LDA (2 M in THF, 223 mL) at −78° C. under N₂. The mixture was stirred at −78° C. under N₂ for 0.5 h. To the resulting solution was added N-methoxy-N-methyl-acetamide (35.57 mL, 335 mmol). The resulting mixture was stirred at −78° C. for 3 h. The reaction mixture was quenched by saturated aqueous NH₄Cl (200 mL) and adjusted to pH˜5 using 2M HCl. The mixture was extracted with EtOAc (5×200 mL). The combined organic layers were washed with brine (3×200 mL), dried over anhydrous Na₂SO₄, filtered and the filtrate concentrated under reduced pressure to give a residue. The residue was purified by column chromatography (10-50% EtOAc in petroleum ether) to give the title compound as a yellow solid. Y=23%. ¹H NMR (400 MHz, DMSO-d₆) δ 13.47 (s, 1H), 8.03 (d, J=2 Hz, 1H), 6.95 (d, J=2 Hz, 1H), 2.55 (s, 3H).

Step 2. 7-Methyl-5H-furo[2,3-d]pyridazin-4-one. To a solution of 2-acetylfuran-3-carboxylic acid (8.0 g, 51.9 mmol) in n-BuOH (100 mL) was added NH₂NH₂·H₂O (50.5 mL, 1.04 mol) and TsOH (17.9 g, 104 mmol) at 25° C. The mixture was stirred at 140° C. for 12 h. The reaction mixture was cooled to room temperature and the precipitated solid was collected and dried to give the title compound as a white solid. Y=58%. ¹H NMR (400 MHz, DMSO-d₆) δ 12.63 (br. s, 1H), 8.20 (d, J=2 Hz, 1H), 7.11 (d, J=2 Hz, 1H), 2.44 (s, 3H).

Step 3. 2-Bromo-7-methyl-5H-furo[2,3-d]pyridazin-4-one. To a solution of 7-methyl-5H-furo[2,3-d]pyridazin-4-one (4.3 g, 28.6 mmol) in THE (50 mL) was added n-BuLi (2.5 M in n-hexane, 22.9 mL, 45.8 mmol) at −78° C. under N₂. The mixture was stirred at −78° C. under N₂ for 0.5 h. To the solution was added 1,2-dibromo-1,1,2,2-tetrachloroethane (3.44 mL, 28.6 mmol). The resulting mixture was stirred at −78° C. for 2 h. The reaction mixture was quenched by addition of H₂O (30 mL) at 0° C., and the resulting mixture was extracted with EtOAc (3×20 mL). The combined organic layers were washed with brine (20 mL), dried over anhydrous Na₂SO₄, filtered and concentrated under reduced pressure to give a residue. The residue was purified by column chromatography (100% ethyl acetate) to give the title compound as a yellow solid. Y=12%. ¹H NMR (400 MHz, DMSO-d₆) δ 12.74 (s, 1H), 7.32 (s, 1H), 2.42 (s, 3H).

Step 4. Ethyl 2-(2-bromo-7-methyl-4-oxo-furo[2,3-d]pyridazin-5-yl)acetate. To a solution of 2-bromo-7-methyl-5H-furo[2,3-d]pyridazin-4-one (800 mg, 3.49 mmol) in DMF (20 mL) was added K₂CO₃ (1.45 g, 10.5 mmol) and ethyl 2-bromoacetate (444 μL, 4.02 mmol) at 25° C. The mixture was stirred at 80° C. for 12 h. The reaction mixture was diluted with H₂O (20 mL) and the resulting mixture was extracted with ethyl acetate (3×20 mL). The combined organic layers were washed with brine (20 mL), dried over anhydrous Na₂SO₄, filtered and concentrated under reduced pressure to give the title compound as a yellow solid. Y=quantitative. ¹H NMR (400 MHz, DMSO-d₆) δ 7.40 (s, 1H), 4.89 (s, 2H), 4.15 (q, J=7 Hz, 2H), 2.46 (s, 3H), 1.20 (t, J=7 Hz, 3H).

Step 5. Ethyl 2-(2-cyclopropyl-7-methyl-4-oxo-furo[2,3-d]pyridazin-5-yl)acetate. To a solution of ethyl 2-(2-bromo-7-methyl-4-oxo-furo[2,3-d]pyridazin-5-yl)acetate (1.1 g, 3.49 mmol) in toluene (20 mL) and H₂O (2 mL) was added cyclopropylboronic acid (450 mg, 5.24 mmol), Pd(OAc)₂ (39 mg, 175 μmol, 0.05 eq), P(Cy)₃ (98 mg, 349 μmol) and K₃PO₄ (2.59 g, 12.2 mmol) at 25° C. under N₂. The mixture was stirred at 80° C. for 12 h. The reaction mixture was diluted with H₂O (20 mL) and extracted with ethyl acetate (3×20 mL). The combined organic layers were washed with brine (20 mL), dried over anhydrous Na₂SO₄, filtered and concentrated under reduced pressure to give a residue. The residue was purified by column chromatography (10-50% EtOAc in petroleum ether) to the title compound as a yellow oil. Y=45%. ¹H NMR (400 MHz, DMSO-d₆) δ 6.80 (s, 1H), 4.86 (s, 2H), 4.14 (q, J=7 Hz, 2H), 2.43 (s, 3H), 2.22-2.14 (m, 1H), 1.19 (t, J=7 Hz, 3H), 1.10-1.04 (m, 2H), 0.99-0.93 (m, 2H).

Step 6. 2-(2-Cyclopropyl-7-methyl-4-oxo-furo[2,3-d]pyridazin-5-yl)acetic acid. To a solution of ethyl 2-(2-cyclopropyl-7-methyl-4-oxo-furo[2,3-d]pyridazin-5-yl)acetate (430 mg, 1.56 mmol) in THE (10 mL) and H₂O (2.5 mL) was added LiOH·H₂O (196 mg, 4.67 mmol) at 25° C. The reaction mixture was stirred at 25° C. for 2 h. The pH was adjusted to ˜5 using 2 M HCl and the mixture extracted with ethyl acetate (3×5 mL). The combined organic layers were washed with brine (5 mL), dried over anhydrous Na₂SO₄, filtered and concentrated under reduced pressure to give the title compound as a yellow solid. Y=quantitative. ¹H NMR (400 MHz, DMSO-d₆) δ 12.97 (br. s, 1H), 6.80 (s, 1H), 4.76 (s, 2H), 2.43 (s, 3H), 2.22-2.13 (m, 1H), 1.10-1.02 (m, 2H), 0.99-0.91 (m, 2H).

### Intermediate D2. 2-{2-Cyclopropyl-4-methyl-7-oxo-6H,7H-thieno[2,3-d]pyridazin-6-yl}acetic acid

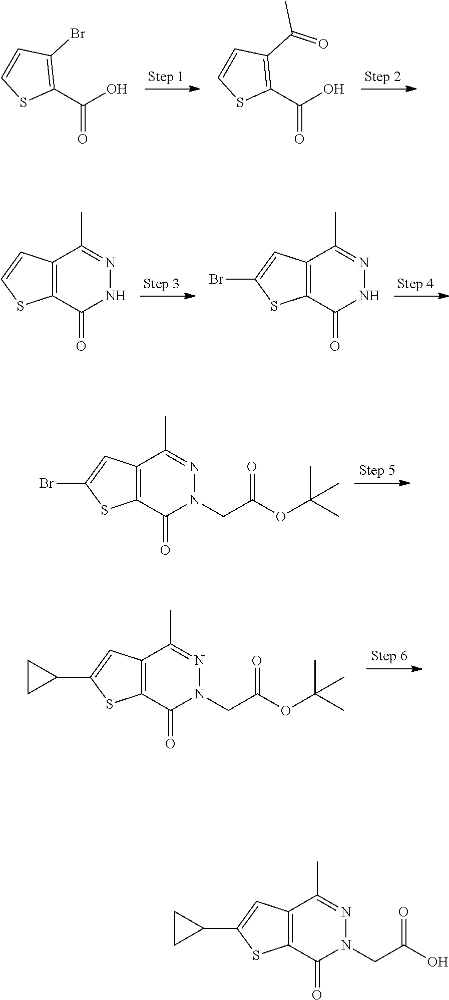

Step 1. 3-Acetylthiophene-2-carboxylic acid. To a solution of 3-bromothiophene-2-carboxylic acid (20 g, 96.6 mmol) in THE (200 mL) was added n-BuLi (2.5 M in hexane, 116 mL, 290 mmol) at −78° C. under nitrogen. The reaction mixture was stirred at −78° C. for 30 min. To the resulting solution was added N-methoxy-N-methyl-acetamide (12.32 mL, 116 mmol). The mixture was stirred at −78° C. for 2 h. The reaction mixture was quenched by saturated aqueous NH₄Cl (100 mL) solution at 0° C., then the resulting mixture was adjusted to pH˜2 with 1 M HCl and extracted with EtOAc (3×100 mL). The combined organic layers were dried over Na₂SO₄, filtered and concentrated under reduced pressure to give the title compound as a yellow solid. Y=52%. ¹H NMR (400 MHz, DMSO-d₆) δ 13.17 (br. s, 1H), 7.94-7.79 (m, 1H), 7.79-7.68 (m, 1H), 2.50 (s, 3H).

Step 2. 4-Methyl-6H-thieno [2,3-d]pyridazin-7-one. To a solution of 3-acetylthiophene-2-carboxylic acid (16.5 g, 97.0 mmol) in EtOH (165 mL) was added NH₂NH₂·H₂O (96.2 mL, 1.94 mol) at 25° C. The reaction mixture was stirred at reflux for 12 h. The reaction mixture was filtered and the solid dried under reduced pressure to give the title compound as a white solid. Y=56%. ¹H NMR (400 MHz, DMSO-d₆) δ 12.56 (br. s, 1H), 8.23 (d, J=5 Hz, 1H), 7.59 (d, J=5 Hz, 1H), 2.50 (s, 3H).

Step 3. 2-Bromo-4-methyl-6H-thieno[2,3-d]pyridazin-7-one. To a solution of 4-methyl-6H-thieno[2,3-d]pyridazin-7-one (3.8 g, 22.9 mmol) in THE (55 mL) was added n-BuLi (2.5 M in hexane, 18.3 mL, 36.6 mmol) at −78° C. The RM was stirred at −78° C. for 1 h, then treated with 1,2-dibromo-1,1,2,2-tetrafluoroethane (17.8 g, 68.6 mmol). The resulting solution was stirred at −78° C. for a further 2 h under N₂. The reaction mixture was quenched by addition of saturated aqueous NH₄Cl solution (20 mL) at 0° C., and then the resulting mixture was diluted with H₂O (20 mL) and extracted with EtOAc (3×30 mL). The combined organic layers were washed with brine (2×20 mL), dried over Na₂SO₄, filtered and concentrated under reduced pressure to give a residue. The residue was purified by prep-TLC (SiO₂, 1:1 petroleum ether/ethyl acetate) to give the title compound as a white solid. Y=32%. ¹H NMR (400 MHz, DMSO-d₆) δ 12.78 (s, 1H), 7.83 (s, 1H), 2.45 (s, 3H).

Step 4. Tert-butyl 2-(2-bromo-4-methyl-7-oxo-thieno[2,3-d]pyridazin-6-yl)acetate. To a mixture of 2-bromo-4-methyl-6H-thieno[2,3-d]pyridazin-7-one (650 mg, 2.65 mmol) and Cs₂CO₃ (2.59 g, 7.96 mmol) in DMF (6.5 mL) was added tert-butyl 2-bromoacetate (588 μL, 3.98 mmol) at 25° C. The mixture was stirred at 80° C. for 12 h. The reaction mixture was diluted with H₂O (7 mL) and the resulting mixture was extracted with EtOAc (3×7 mL). The combined organic layers were washed with brine (7 mL), dried over Na₂SO₄, filtered and concentrated under reduced pressure to give a residue. The residue was purified by column chromatography (25% EtOAc in petroleum ether) to give the title compound as a white solid. Y=63%.

Step 5. Tert-butyl 2-(2-cyclopropyl-4-methyl-7-oxo-thieno[2,3-d]pyridazin-6-yl)acetate. To a solution of tert-butyl 2-(2-bromo-4-methyl-7-oxo-thieno[2,3-d]pyridazin-6-yl)acetate (470 mg, 1.31 mmol) in toluene (5 mL) and H₂O (0.25 mL) was added Pd(OAc)₂ (14.7 mg, 65.4 μmol), K₃PO₄ (972 mg, 4.58 mmol), P(Cy)₃ (42.4 μL, 131 μmol) and cyclopropylboronic acid (112 mg, 1.31 mmol) at 25° C. The mixture was stirred at reflux for 2 h. The reaction mixture was diluted with H₂O (5 mL) and the resulting mixture was extracted with EtOAc (3×5 mL). The combined organic layers were washed with brine (5 mL), dried over Na₂SO₄, filtered and concentrated under reduced pressure to give a residue. The residue was purified by column chromatography (50% EtOAc in petroleum ether) to give the title compound as a white solid. Y=72%.

Step 6. 2-(2-Cyclopropyl-4-methyl-7-oxo-thieno[2,3-d]pyridazin-6-yl)acetic acid. To a solution of tert-butyl 2-(2-cyclopropyl-4-methyl-7-oxo-thieno[2,3-d]pyridazin-6-yl)acetate (260 mg, 811 μmol) in DCM (2.6 mL) was added TFA (541 μL, 7.30 mmol) at 0° C. The mixture was stirred at 25° C. for 12 h. The reaction mixture was concentrated under reduced pressure to give the title compound as a white solid. Y=98%.

### Intermediate D3. 2-{2-Cyclopropyl-7-ethyl-4-oxo-4H,5H-furo[2,3-d]pyridazin-5-yl}acetic acid

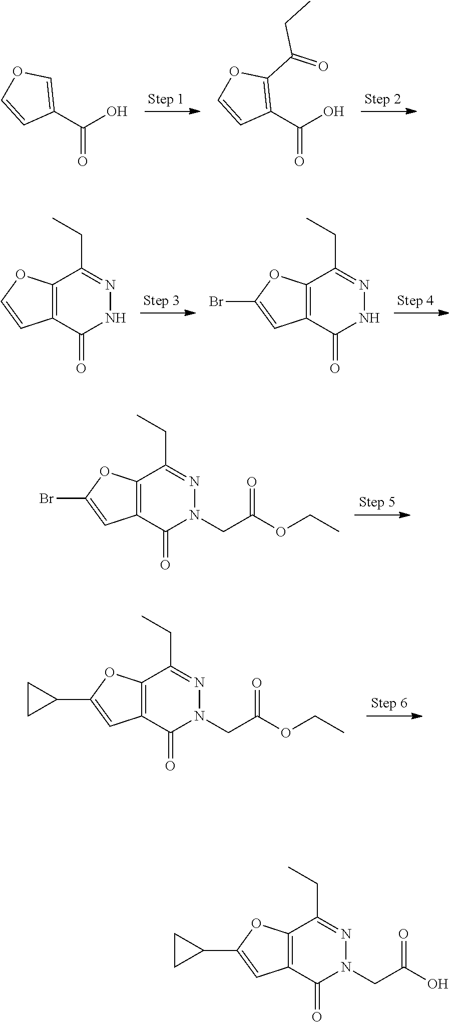

Step 1. 2-Propanoylfuran-3-carboxylic acid. To a solution of furan-3-carboxylic acid (5.0 g, 44.6 mmol) in THE (50 mL) was added dropwise LDA (2 M in THF, 44.6 mL, 89.2 mmol) at −78° C. The resulting solution was stirred at −78° C. for 0.5 h, then treated with N-methoxy-N-methyl-propanamide (7.84 g, 66.9 mmol). The mixture was stirred at −78° C. for 1 h under N₂. The reaction mixture was quenched with saturated aqueous NH₄Cl (50 mL) and the resulting mixture adjusted to pH˜5 with aqueous 2 M HCl. The mixture was extracted with EtOAc (5×70 mL). The combined organic layers were washed with brine (3×70 mL), dried over anhydrous Na₂SO₄, filtered and the filtrate concentrated under reduced pressure to give a residue. The residue was purified by column chromatography (10-50% EtOAc in petroleum ether) to give the title compound as a yellow solid. Y=41%. ¹H NMR (400 MHz, DMSO-d₆) δ 13.45 (s, 1H), 8.01 (d, J 2 Hz, 1H), 6.94 (d, J 2 Hz, 1H), 3.00 (q, J 8 Hz, 2H), 1.06 (t, J 8 Hz, 3H).

Step 2. 7-ethyl-5H-furo[2,3-d]pyridazin-4-one. To a solution of 2-propanoylfuran-3-carboxylic acid (3.0 g, 17.8 mmol) in n-BuOH (30 mL) was added TsOH (6.14 g, 35.7 mmol) and N₂H₄·H₂O (17.70 mL, 357 mmol) at 25° C. The mixture was stirred at reflux for 12 h under N₂. The reaction mixture was cooled to room temperature and filtered, the filter cake was dried under reduced pressure to give the title compound as a white solid. Y=55%. ¹H NMR (400 MHz, DMSO-d₆) δ 12.65 (s, 1H), 8.20 (d, J 2 Hz, 1H), 7.12 (d, J 2 Hz, 1H), 2.85 (q, J 8 Hz, 2H), 1.26 (t, J 8 Hz, 3H).

Step 3. 2-bromo-7-ethyl-5H-furo[2,3-d]pyridazin-4-one. To a solution of 1,2-dibromo-1,1,2,2-tetrachloroethane (1.46 mL, 12.2 mmol) in THE (10 mL) was added n-BuLi (2.5 M in n-hexane, 6.1 mL, 15.3 mmol) at −78° C., and the solution was stirred at −78° C. for 0.5 h. To the resulting solution was added 7-ethyl-5H-furo[2,3-d]pyridazin-4-one (1.0 g, 6.09 mmol) at −78° C., then the solution was stirred at 25° C. for 1 h under N₂. The mixture was quenched by addition of saturated aqueous NH₄Cl (10 mL) and the resulting mixture was extracted with EtOAc (3×10 mL). The combined organic layers were washed with brine (3×10 mL), dried over Na₂SO₄, filtered and concentrated under reduced pressure to give a residue. The residue was purified by prep-HPLC (column: Phenomenex Luna 80×30 mm 3 μm; mobile phase: [water (HCl)−ACN]; B: 20-40%, 8 min) and lyophilised to give the title compound as a white solid. Y=14%. ¹H NMR (400 MHz, DMSO-d₆) δ 12.76 (s, 1H), 7.31 (s, 1H), 2.81 (q, J 8 Hz, 2H), 1.24 (t, J 8 Hz, 3H).

Step 4. Ethyl 2-(2-bromo-7-ethyl-4-oxo-furo[2,3-d]pyridazin-5-yl)acetate. To a solution of 2-bromo-7-ethyl-5H-furo[2,3-d]pyridazin-4-one (200 mg, 0.82 mmol) in DMF (1.6 mL) was added ethyl 2-bromoacetate (100 μL, 0.91 mmol) and Cs₂CO₃ (804 mg, 2.47 mmol) at 25° C. The mixture was stirred at 80° C. for 1 h under N₂. The mixture was diluted with H₂O (2 mL) and the resulting mixture was extracted with EtOAc (3×2 mL). The combined organic layers were washed with brine (2×6 mL), dried over Na₂SO₄, filtered and concentrated under reduced pressure to give the title compound as a white solid. Y=81%. ¹H NMR (400 MHz, DMSO-d₆) δ 7.39 (s, 1H), 4.89 (s, 2H), 4.16-4.13 (m, 2H), 2.83 (q, J=8 Hz, 2H), 1.24-1.20 (m, 6H).

Step 5. Ethyl 2-(2-cyclopropyl-7-ethyl-4-oxo-furo[2,3-d]pyridazin-5-yl)acetate. To a solution of cyclopropylboronic acid (69 mg, 0.80 mmol) in toluene (4 mL) and H₂O (0.4 mL) was added ethyl 2-(2-bromo-7-ethyl-4-oxo-furo[2,3-d]pyridazin-5-yl)acetate (0.22 g, 0.67 mmol), K₃PO₄ (497 mg, 2.34 mmol), P(Cy)₃ (21.7 μL, 67 μmol) and Pd(OAc)₂ (7.5 mg, 33.4 μmol) at 25° C. The mixture was stirred at 80° C. for 12 h under N₂. The reaction mixture was diluted with H₂O (4 mL) and the resulting mixture was extracted with EtOAc (3×4 mL). The combined organic layers were washed with brine (4 mL), dried over Na₂SO₄, filtered and concentrated under reduced pressure to give a residue. The residue was purified by prep-TLC (SiO₂, 1:5 EtOAc in petroleum ether) to give the title compound as a white solid. Y=41%.

¹H NMR (400 MHz, DMSO-d₆) δ 6.80 (s, 1H), 4.91-4.82 (m, 2H), 4.14 (q, J 8 Hz, 2H), 2.89-2.75 (m, 2H), 2.25-2.12 (m, 1H), 1.30-1.15 (m, 6H), 1.10-1.04 (m, 2H), 1.00-0.93 (m, 2H).

Step 6. 2-(2-Cyclopropyl-7-ethyl-4-oxo-furo[2,3-d]pyridazin-5-yl)acetic acid. To a solution ethyl 2-(2-cyclopropyl-7-ethyl-4-oxo-furo[2,3-d]pyridazin-5-yl)acetate (80 mg, 0.28 mmol) in THE (0.8 mL) and H₂O (0.2 mL) was added LiOH·H₂O (34.7 mg, 0.83 mmol) at 25° C. The reaction mixture was stirred at 25° C. for 2 h. The mixture was diluted with H₂O (1 mL) and the resulting mixture was extracted with EtOAc (3×1 mL). The aqueous layer was adjusted to pH ˜5 with 2 M HCl and the resulting mixture extracted with EtOAc (3×3 mL). The combined organic layers were washed with brine (2×10 mL), dried over Na₂SO₄, filtered and concentrated under reduced pressure to give the title compound as a white solid. Y=61%. ¹H NMR (400 MHz, DMSO-d₆) δ 12.98 (br. s, 1H), 6.79 (s, 1H), 4.76 (s, 2H), 2.82 (q, J 8 Hz, 2H), 2.22-2.14 (m, 1H), 1.24 (t, J 8 Hz, 3H), 1.09-1.04 (m, 2H), 0.98-0.92 (m, 2H).

### Intermediate D4. Ethyl 2-(2-bromo-4-isopropyl-7-oxo-thieno[2,3-d]pyridazin-6-yl)acetate

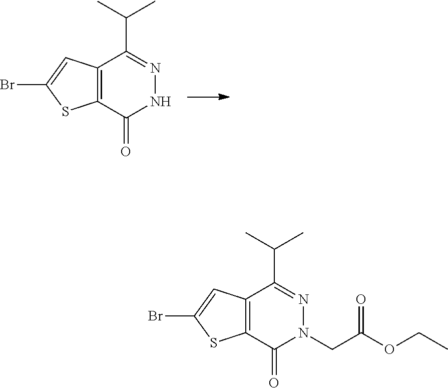 [SMILES: CC(C)c1n[nH]c(=O)c2sc(Br)cc12 ; CCOC(=O)Cn1nc(C(C)C)c2cc(Br)sc2c1=O]

To a mixture of 2-bromo-4-isopropyl-6H-thieno[2,3-d]pyridazin-7-one (Intermediate C19, 1.0 g, 3.66 mmol) and Cs₂CO₃ (3.58 g, 11.0 mmol) in DMF (10 mL) was added ethyl 2-bromoacetate (526 μL 4.76 mmol) at 25° C. The mixture was stirred at 80° C. for 2 h. The reaction mixture was allowed to cool to rt, diluted with H₂O (10 mL) and extracted with EtOAc (3×10 mL). The combined organic layers were washed with brine (10 mL), dried over anhydrous Na₂SO₄, filtered and concentrated under reduced pressure to give a residue. The residue was purified by column chromatography (25% EtOAc in petroleum ether) to give the title compound as a white solid. Y=76%. ¹H NMR (400 MHz, DMSO-d₆) δ 8.00 (s, 1H), 4.91 (s, 2H), 4.25-4.10 (m, 2H), 3.35-3.32 (m, 1H), 1.23 (d, J=7 Hz, 6H), 1.25-1.15 (m, 3H).

### Intermediate D5. Ethyl 2-[2-bromo-4-oxo-7-(propan-2-yl)-4H,5H-furo[2,3-d]pyridazin-5-yl]acetate

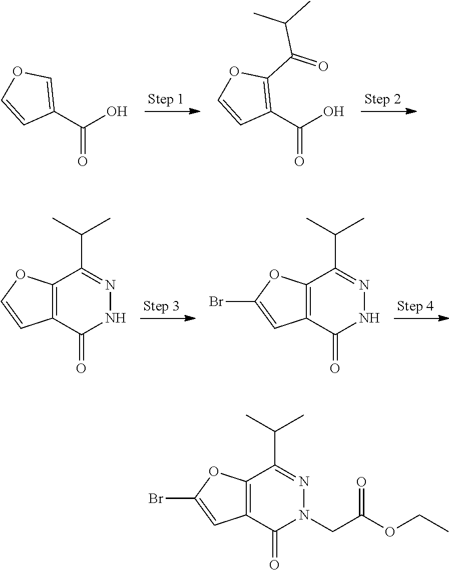 [SMILES: O=C(O)c1ccoc1 ; CC(C)C(=O)c1occc1C(=O)O ; CC(C)c1n[nH]c(=O)c2ccoc12 ; CC(C)c1n[nH]c(=O)c2cc(Br)oc12 ; CCOC(=O)Cn1nc(C(C)C)c2oc(Br)cc2c1=O]

Step 1. 2-(2-Methylpropanoyl)furan-3-carboxylic acid. To a solution of furan-3-carboxylic acid (3.0 g, 26.8 mmol) in THE (50 mL) at −78° C. was added LDA (2 M in THF, 26.8 mL, 53.6 mmol), and the solution stirred at −78° C. for 0.5 h. The RM was treated with N-methoxy-N,2-dimethyl-propanamide (5.27 g, 40.2 mmol) at −78° C. and the mixture stirred at −78° C. for 2 h. The mixture was quenched with saturated aqueous NH₄Cl (30 mL) and the resulting mixture extracted with EtOAc (5×30 mL). The combined organic layers were washed with brine (3×30 mL), dried over anhydrous Na₂SO₄, filtered and concentrated under reduced pressure to give a residue. The residue was purified by column chromatography (100% EtOAc) to give the title compound as a white solid. Y=92%. ¹H NMR (400 MHz, DMSO-d₆) δ 7.79 (d, J=8 Hz, 1H), 7.51 (d, J=8 Hz, 1H), 3.65-3.49 (m, 1H), 1.65-0.96 (m, 6H).

Step 2. 7-isopropyl-5H-furo[2,3-d]pyridazin-4-one. To a solution of 2-(2-methylpropanoyl)furan-3-carboxylic acid (2.0 g, 11.0 mmol) in n-BuOH (30 mL) at 25° C. were added NH₂NH₂·H₂O (10.9 mL, 220 mmol) and TsOH (3.78 g, 22.0 mmol). The reaction mixture was stirred at reflux for 12 h. The reaction mixture was diluted with H₂O (20 mL) and extracted with ethyl acetate (3×20 mL). The combined organic layers were washed with brine (20 mL), dried over anhydrous Na₂SO₄, filtered and concentrated under reduced pressure to give the title compound (2.0 g) as a yellow solid, used as is.

Step 3. 2-Bromo-7-isopropyl-5H-furo[2,3-d]pyridazin-4-one. To a solution of 7-isopropyl-5H-furo[2,3-d]pyridazin-4-one (800 mg, 4.49 mmol) in THE (15 mL) at −78° C. was added n-BuLi (2.5 M in THF, 3.59 mL, 7.18 mmol). The solution was stirred at −78° C. for 0.5 h, then treated with 1,2-dibromo-1,1,2,2-tetrachloroethane (647 μL, 5.39 mmol). The solution was stirred at 25° C. for 2 h. The reaction mixture was cooled to 0° C. and quenched by addition of H₂O (10 mL). The resulting mixture was extracted with EtOAc (3×20 mL). The combined organic layers were washed with brine (2×20 mL), dried over anhydrous Na₂SO₄, filtered and concentrated under reduced pressure to give a residue. The residue was purified by column chromatography (10-50% EtOAc in petrol) to give the title compound as a yellow solid. Y=24%. ¹H NMR (400 MHz, DMSO-d₆) δ 7.32 (s, 1H), 3.27-3.18 (m, 1H), 1.27 (d, J=7 Hz, 6H).

Step 4. Ethyl 2-[2-bromo-4-oxo-7-(propan-2-yl)-4H,5H-furo[2,3-d]pyridazin-5-yl]acetate. To a solution of 2-bromo-7-isopropyl-5H-furo[2,3-d]pyridazin-4-one (700 mg, 2.72 mmol) in DMF (10 mL) at 25° C. were added K₂CO₃ (1.13 g, 8.17 mmol) and ethyl 2-bromoacetate (361 μL, 3.27 mmol). The mixture was stirred at 80° C. for 3 h. The reaction mixture was diluted with H₂O (10 mL) and extracted with ethyl acetate (3×10 mL). The combined organic layers were washed with brine (10 mL), dried over anhydrous Na₂SO₄, filtered and concentrated under reduced pressure to give the title compound (1.0 g) as a yellow oil, used as is. ¹H NMR (400 MHz, DMSO-d₆) δ 7.39 (s, 1H), 4.89 (s, 2H), 4.15 (q, J=7 Hz, 2H), 3.30-3.21 (m, 1H), 1.29 (d, J=7 Hz, 6H), 1.20 (t, J=7 Hz, 3H).

### Intermediate D6. 2-[2-Cyclopropyl-4-oxo-7-(propan-2-yl)-4H,5H-furo[2,3-d]pyridazin-5-yl]acetic acid

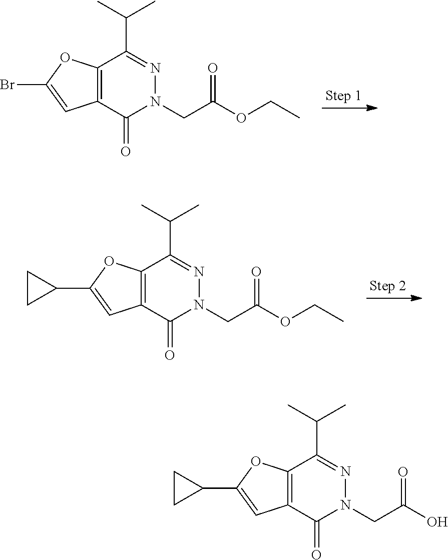 [SMILES: CCOC(=O)Cn1nc(C(C)C)c2oc(Br)cc2c1=O ; CCOC(=O)Cn1nc(C(C)C)c2oc(C3CC3)cc2c1=O ; CC(C)c1nn(CC(=O)O)c(=O)c2cc(C3CC3)oc12]

Step 1. Ethyl 2-(2-cyclopropyl-7-isopropyl-4-oxo-furo[2,3-d]pyridazin-5-yl)acetate. To a solution of ethyl 2-(2-bromo-7-isopropyl-4-oxo-furo[2,3-d]pyridazin-5-yl)acetate (Intermediate D5, 1.0 g, 2.91 mmol) in toluene (20 mL) and H₂O (2 mL) at 25° C. were added cyclopropylboronic acid (375 mg, 4.37 mmol), Pd(OAc)₂ (33 mg, 0.15 mmol), P(Cy)₃ (94 μL, 0.29 mmol) and K₃PO₄ (2.16 g, 10.2 mmol). The reaction mixture was stirred at 80° C. for 2 h. The reaction mixture was diluted with H₂O (20 mL) and the resulting mixture extracted with ethyl acetate (3×20 mL). The combined organic layers were washed with brine (20 mL), dried over anhydrous Na₂SO₄, filtered and concentrated under reduced pressure to give a residue. The residue was purified by column chromatography (10-50% EtOAc in petrol) to give the title compound as a yellow gum. Y=68%. ¹H NMR (400 MHz, DMSO-d₆) δ 6.80 (s, 1H), 4.86 (s, 2H), 4.14 (q, J=7 Hz, 2H), 3.27-3.15 (m, 1H), 2.26-2.14 (m, 1H), 1.29 (d, J=7 Hz, 6H), 1.19 (t, J=7 Hz, 3H), 1.11-1.04 (m, 2H), 1.00-0.93 (m, 2H).

Step 2. 2-[2-Cyclopropyl-4-oxo-7-(propan-2-yl)-4H,5H-furo[2,3-d]pyridazin-5-yl]acetic acid. To a solution of ethyl 2-(2-cyclopropyl-7-isopropyl-4-oxo-furo[2,3-d]pyridazin-5-yl)acetate (150 mg, 0.49 mmol) in THE (2 mL) and H₂O (0.4 mL) at 25° C. was added LiOH·H₂O (62 mg, 1.48 mmol) and the solution was stirred at 25° C. for 2 h. The reaction mixture was adjusted to pH ˜4 with 2M HCl and the resulting mixture extracted with ethyl acetate (3×3 mL). The combined organic layers were washed with brine (3 mL), dried over anhydrous Na₂SO₄, filtered and concentrated under reduced pressure to give the title compound as a white solid. Y=40%.

### Intermediate D7. Ethyl 2-(2-chloro-4-isopropyl-7-oxo-thieno[2,3-d]pyridazin-6-yl)acetate

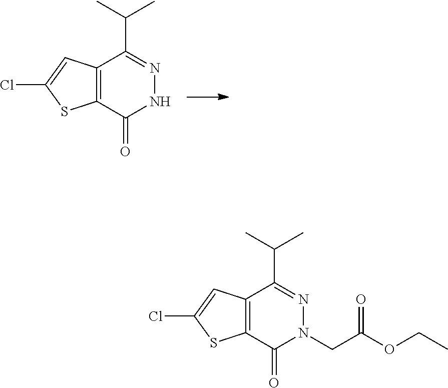 [SMILES: CC(C)c1n[nH]c(=O)c2sc(Cl)cc12 ; CCOC(=O)Cn1nc(C(C)C)c2cc(Cl)sc2c1=O]

Prepared in an analogous way to Intermediate D4 using Intermediate C9 and ethyl bromoacetate to give the title compound as a white solid. Y=81%. ¹H NMR (400 MHz, DMSO-d₆) δ 7.91 (s, 1H), 4.91 (s, 2H), 4.16 (q, J=7 Hz, 2H), 3.39-3.33 (m, 1H), 1.24 (d, J=7 Hz, 6H), 1.20 (t, J=7 Hz, 3H).

### Intermediate D8. Ethyl 2-{2-bromo-4-cyclopropyl-7-oxo-6H,7H-thieno[2,3-d]pyridazin-6-yl}acetate

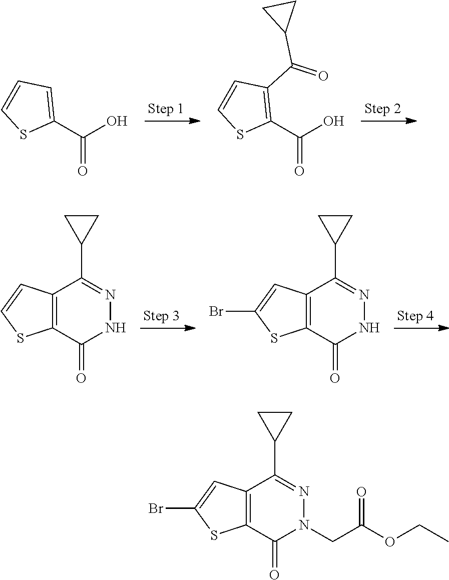 [SMILES: O=C(O)c1cccs1 ; O=C(O)c1sccc1C(=O)C1CC1 ; O=c1[nH]nc(C2CC2)c2ccsc12 ; O=c1[nH]nc(C2CC2)c2cc(Br)sc12 ; CCOC(=O)Cn1nc(C2CC2)c2cc(Br)sc2c1=O]

Step 1. 3-(cyclopropanecarbonyl)thiophene-2-carboxylic acid. To a solution of thiophene-2-carboxylic acid (30 g, 234 mmol) in THE (300 mL) at −78° C. under N₂ was added dropwise n-BuLi (2.5 M in hexane, 187 mL, 468 mmol). The mixture was stirred at −78° C. for 1 h, then treated dropwise with N-methoxy-N methyl-cyclopropane carboxamide (39.3 g, 304 mmol). The RM was stirred at −78° C. for 2 h. The reaction mixture was quenched by addition of saturated aqueous NH₄Cl (300 mL) at 25° C. The resulting mixture was extracted with dichloromethane (3×300 mL). The aqueous phase was acidified to pH ˜3 with 2 M HCl and diluted with H₂O (300 mL). This mixture was extracted with dichloromethane (3×300 mL). The combined organic phase was washed with brine (300 mL), dried over Na₂SO₄, filtered and concentrated under reduced pressure to give the title compound as a white solid. Y=37%. ¹H NMR (400 MHz, DMSO-d₆) δ 13.80 (br. s, 1H), 7.87 (d, J=5 Hz, 1H), 7.25 (d, J=5 Hz, 1H), 2.5-2.40 (m, 1H), 1.10-1.00 (m, 4H).

Step 2. 4-Cyclopropyl-6H-thieno [2,3-d]pyridazin-7-one. To a solution of 3-(cyclopropane carbonyl)thiophene-2-carboxylic acid (13 g, 66.3 mmol) in EtOH (130 mL) was added NH₂NH₂·H₂O (65.7 mL, 1.33 mol). The mixture was stirred at reflux under N₂ for 2 h. The reaction mixture was concentrated under reduced pressure to remove EtOH. The residue was diluted with H₂O (130 mL) and the resulting mixture extracted with ethyl acetate (3×130 mL). The combined organic layers were washed with brine (130 mL), dried over anhydrous Na₂SO₄, filtered and concentrated under reduced pressure to give the title compound as a white solid. Y=79%. ¹H NMR (400 MHz, DMSO-d₆) δ 8.26 (d, J=5 Hz, 1H), 7.82 (d, J=5 Hz, 1H), 2.44-2.34 (m, 1H), 1.01-0.90 (m, 4H).

Step 3. 2-Bromo-4-cyclopropyl-6H-thieno[2,3-d]pyridazin-7-one. To a solution of 4-cyclopropyl-6H-thieno[2,3-d]pyridazin-7-one (5.0 g, 26.0 mmol) in THE (50 mL) at −78° C. was added LDA (2 M in THF, 32.5 mL, 65 mmol). The RM was stirred at −78° C. under nitrogen for 0.5 h, then treated with 1,2-dibromo-1,1,2,2-tetrachloro-ethane (3.75 mL, 31.2 mmol. The solution was stirred at 25° C. under N₂ for 2 h. The reaction mixture was quenched by addition of saturated aqueous NH₄Cl (50 mL). The reaction mixture was diluted with H₂O (50 mL) and the resulting mixture was extracted with ethyl acetate (3×50 mL). The combined organic layers were washed with brine (50 mL), dried over Na₂SO₄, filtered and concentrated under reduced pressure to give a residue. The residue was purified by prep-HPLC (column: Agela DuraShell C18 250×70 mm×10 μm; mobile phase: [water (NH₄HCO₃)−ACN]; B: 27-50%, 20 min) and lyophilised to give the title compound as a white solid. Y=30%. ¹H NMR (400 MHz, DMSO-d₆) δ 12.86-12.57 (m, 1H), 8.05 (s, 1H), 2.39-2.29 (m, 1H), 1.01-0.85 (m, 4H).

Step 4. Ethyl 2-{2-bromo-4-cyclopropyl-7-oxo-6H, 7H-thieno[2,3-d]pyridazin-6-yl}acetate. To a solution of 2-bromo-4-cyclopropyl-6H-thieno[2,3-d]pyridazin-7-one (650 mg, 2.40 mmol) in DMF (6.5 mL) at 25° C. under N₂ were added Cs₂CO₃ (2.34 g, 7.19 mmol) and ethyl 2-bromoacetate (345 μL, 3.12 mmol). The mixture was stirred at 80° C. for 2 h under N₂. The reaction mixture was diluted with H₂O (6.5 mL) and the resulting mixture extracted with ethyl acetate (3×6.5 mL). The combined organic layers were washed with brine (6.5 mL), dried over Na₂SO₄, filtered and concentrated under reduced pressure to give a residue. The residue was purified by column chromatography (50% EtOAc in petrol) to give the title compound as a white solid. Y=91%.

### Intermediate D9. Ethyl 2-{2-bromo-4-ethyl-7-oxo-6H,7H-thieno[2,3-d]pyridazin-6-yl}acetate

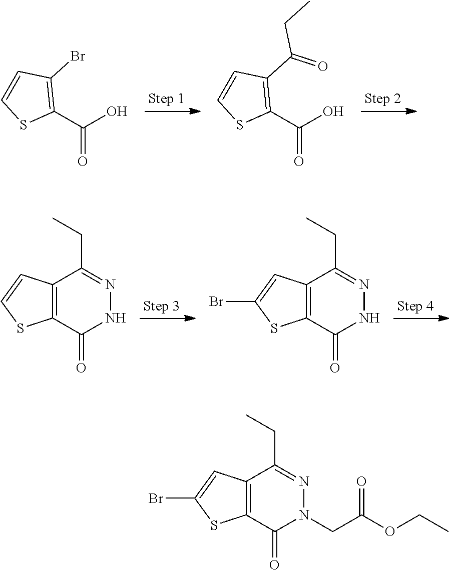 [SMILES: O=C(O)c1sccc1Br ; CCC(=O)c1ccsc1C(=O)O ; CCc1n[nH]c(=O)c2sccc12 ; CCc1n[nH]c(=O)c2sc(Br)cc12 ; CCOC(=O)Cn1nc(CC)c2cc(Br)sc2c1=O]

Step 1. 3-Propanoylthiophene-2-carboxylic acid. To a solution of 3-bromothiophene-2-carboxylic acid (50 g, 241 mmol) in THE (500 mL) at −78° C. was added n-BuLi (2.5 M in hexane, 241 mL, 241 mmol). The mixture was stirred at −78° C. for 30 min, then treated with N-methoxy-N-methyl-propanamide (34.0 g, 290 mmol). The mixture was stirred at −78° C. for 6 h. The reaction mixture was quenched by addition saturated aqueous NH₄Cl (500 mL) and the resulting mixture extracted with EtOAc (3×500 mL). The aqueous phase was adjusted to pH˜2 with 2 M HCl and the resulting mixture extracted with EtOAc (3×500 mL). The organic layers were washed with brine (500 mL), dried over Na₂SO₄, filtered and concentrated under reduced pressure to give a residue. The residue was purified by column chromatography (0-50% EtOAc in petrol) to give the title compound as a yellow solid. Y=67%. ¹H NMR (400 MHz, DMSO-d₆) δ 13.52 (br. s, 1H), 7.88 (d, J=5 Hz, 1H), 7.22 (d, J=5 Hz, 1H), 2.83 (q, J=8 Hz, 2H), 1.05 (t, J=8 Hz, 3H).

Steps 2,3 and 4 were analogous to Intermediate D8 to give the title compound as a white solid. ¹H NMR (400 MHz, DMSO-d₆) δ 7.95 (s, 1H), 4.91 (s, 2H), 4.15 (q, J=8 Hz, 2H), 2.88 (q, J=8 Hz, 2H), 1.23-1.18 (m, 6H).

### Compound 1. N-[(3R)-1-Cyclopropyl-3-piperidyl]-2-(4-isopropyl-1-methyl-7-oxo-pyrazolo[3,4-d]pyridazin-6-yl)acetamide

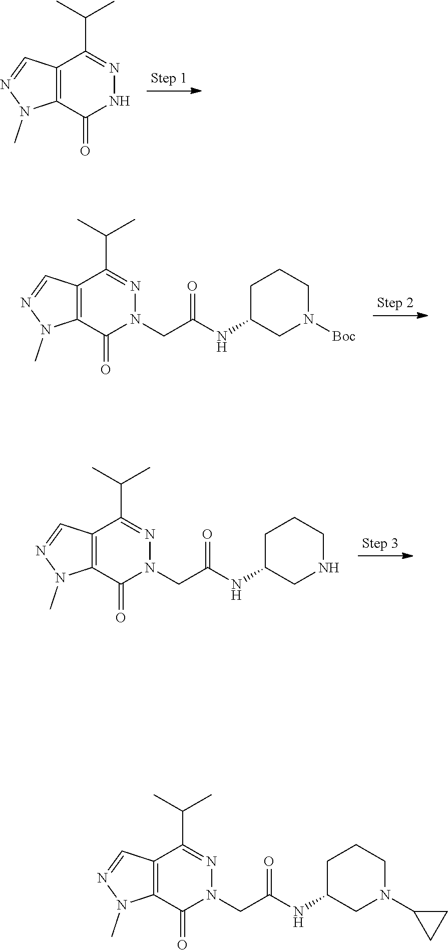

Step 1. Tert-butyl (3R)-3-[[2-(4-isopropyl-1-methyl-7-oxo-pyrazolo[3,4-d]pyridazin-6-yl) acetyl]amino]piperidine-1-carboxylate. To a solution of 4-isopropyl-1-methyl-6H-pyrazolo[3,4-d]pyridazin-7-one (Intermediate C1, 210 mg, 1.09 mmol) in DMF (2.1 mL) at 25° C. was added Cs₂CO₃ (1.07 g, 3.28 mmol) and tert-butyl (3R)-3-[(2-chloroacetyl)amino]piperidine-1-carboxylate (Intermediate B1, 453 mg, 1.64 mmol). The mixture was stirred at 80° C. for 1 h. The RM was concentrated under reduced pressure, diluted with water (10 mL) and extracted with EtOAc (3×10 mL). The combined organic layers were washed with brine (10 mL), dried over Na₂SO₄, filtered and concentrated under reduced pressure to give a residue. Purification by FCC (SiO₂, 2-5% EtOAc in petroleum ether) gave the title compound as a yellow solid. Y=85%. ¹H NMR (400 MHz, DMSO-d₆) δ 8.27 (s, 1H), 8.03 (d, J=8 Hz, 1H), 4.76-4.59 (m, 2H), 4.27 (s, 3H), 3.59-3.51 (m, 3H), 3.21-3.13 (m, 1H), 3.01-2.70 (m, 2H), 1.85-1.75 (m, 1H), 1.71-1.62 (m, 1H), 1.43-1.33 (m, 11H), 1.27 (d, J=7 Hz, 6H).

Step 2. 2-(4-Isopropyl-1-methyl-7-oxo-pyrazolo[3,4-d]pyridazin-6-yl)-N-[(3R)-3-piperidylacetamide hydrochloride. solution of tert-butyl (3R)-3-[[2-(4-isopropyl-1-methyl-7-oxo-pyrazolo[3,4-d]pyridazin-6-yl) acetyl]amino]piperidine-1-carboxylate (400 mg, 925 μmol) in 4M HCl in EtOAc (4 mL) was stirred at 25° C. for 1 h. The reaction mixture was concentrated under reduced pressure to give the title compound as a yellow solid (quantitative), which was used without purification. ¹H NMR (400 MHz, DMSO-d₆) δ 9.36-9.05 (m, 2H), 8.43 (d, J=8 Hz, 1H), 8.28 (s, 1H), 4.70 (s, 2H), 4.27 (s, 3H), 3.99-3.91 (m, 1H), 3.22-3.06 (m, 3H), 2.91-2.66 (m, 2H), 1.88-1.76 (m, 2H), 1.70-1.67 (m, 1H), 1.55-1.42 (m, 1H), 1.28 (d, J=7 Hz, 6H).

Step 3. N-[(3R)-1-Cyclopropyl-3-piperidyl]-2-(4-isopropyl-1-methyl-7-oxo-pyrazolo[3,4-d]pyridazin-6-yl)acetamide. To a solution of 2-(4-isopropyl-1-methyl-7-oxo-pyrazolo[3,4-d]pyridazin-6-yl)-N-[(3R)-3-piperidyl]acetamide hydrochloride (150 mg, 370 μmol) in MeOH (1.5 mL) at 25° C. was added (1-ethoxycyclopropoxy)-trimethyl-silane (409 μL, 2.04 mmol), NaBH₃CN (93.0 mg, 1.48 mmol) and AcOH (16.9 μL, 296 μmol) under N₂. The mixture was stirred at 45° C. for 12 h. The product was collected by filtration and dried in vacuo. The solid was triturated with water (3 mL) and dried in vacuo to give the title compound as a white solid. Y=33%. ¹H NMR (400 MHz, DMSO-d₆) δ 8.27 (s, 1H), 7.84 (d, J=8 Hz, 1H), 4.66 (s, 2H), 4.27 (s, 3H), 3.74-3.52 (m, 1H), 3.21-3.14 (m, 1H), 2.90-2.80 (m, 1H), 2.75-2.65 (m, 1H), 2.25-2.12 (m, 1H), 2.10-2.00 (m, 1H), 1.73-1.54 (m, 3H), 1.46-1.32 (m, 1H), 1.28 (d, J=7 Hz, 6H), 1.25-1.10 (m, 1H), 0.39-0.37 (m, 2H), 0.27-0.17 (m, 2H). LCMS (ESI): m z: [M+H]⁺=373.2.

### Compound 2. N-[(3R)-1-Cyclopropyl-3-piperidyl]-2-(7-isopropyl-1-methyl-4-oxo-pyrazolo[3,4-d]pyridazin-5-yl)acetamide

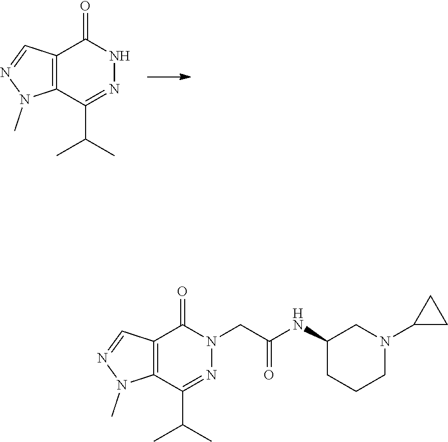 [SMILES: CC(C)c1n[nH]c(=O)c2cnn(C)c12 ; CC(C)c1nn(CC(=O)N[C@@H]2CCCN(C3CC3)C2)c(=O)c2cnn(C)c12]

To a mixture of 7-isopropyl-1-methyl-5H-pyrazolo[3,4-d]pyridazin-4-one (Intermediate C2, 100 mg, 0.52 mmol) and Cs₂CO₃ (339 mg, 1.04 mmol) in DMF (1 mL) at 25° C. was added 2-chloro-N-[(3R)-1-cyclopropyl-3-piperidyl]acetamide (Intermediate A2, 16.9 μL, 1.04 mmol). The RM was stirred at 60° C. for 12 h, diluted with water (1 mL) and extracted with EtOAc (3×1 mL). The combined organic layers were washed with brine (3×1 mL), dried over Na₂SO₄, filtered and concentrated under reduced pressure. Purification by Prep HPLC (column: Phenomenex Gemini NX—C₁₈ (75×30 mm, 3 μm); mobile phase: [water (10 mM NH₄HCO₃)−ACN]; B: 13-43%, 10 min) gave the title compound as a white solid. Y=41%. ¹H NMR (400 MHz, DMSO-d₆) δ 8.20 (s, 1H), 7.83 (d, J=8 Hz, 1H), 4.65 (s, 2H), 4.25 (s, 3H), 3.64-3.59 (m, 1H), 3.59-3.56 (m, 1H), 2.83 (d, J=8 Hz, 1H), 2.69-2.66 (m, 1H), 2.18 (t, J=10 Hz, 1H), 2.04 (t, J=10 Hz, 1H), 1.67-1.61 (m, 1H), 1.60-1.55 (m, 2H), 1.43-1.33 (m, 1H), 1.27 (d, J=6 Hz, 6H), 1.22-1.09 (m, 1H), 0.40-0.35 (m, 2H), 0.26-0.18 (m, 2H). LC-MS (ESI): m z: [M+H]⁺=373.3.

### Compound 3. N-[(3R)-1-Cyclopropyl-3-piperidyl]-2-(7-isopropyl-2-methyl-4-oxo-pyrazolo[3,4-d]pyridazin-5-yl)acetamide

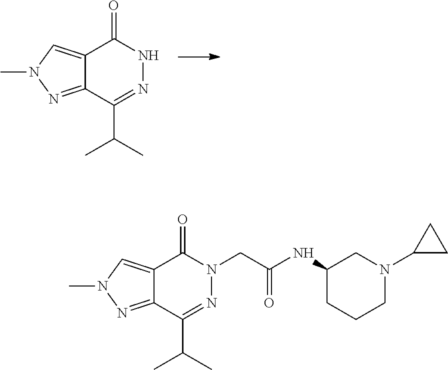 [SMILES: CC(C)c1n[nH]c(=O)c2cn(C)nc12 ; CC(C)c1nn(CC(=O)N[C@@H]2CCCN(C3CC3)C2)c(=O)c2cn(C)nc12]

To a mixture of 7-isopropyl-2-methyl-5H-pyrazolo[3,4-d]pyridazin-4-one (Intermediate C3, 100 mg, 0.52 mmol) and Cs₂CO₃ (339 mg, 1.04 mmol) in DMF (1 mL) at 25° C. was added 2-chloro-N-[(3R)-1-cyclopropyl-3-piperidyl]acetamide (Intermediate A2, 169 mg 0.78 mmol). The mixture was stirred at 60° C. for 12 h. The RM was diluted with water (1 mL) and extracted with EtOAc (3×1 mL). The combined organic layers were washed with brine (3×1 mL), dried over Na₂SO₄, filtered and concentrated under reduced pressure. Purification by Prep HPLC (column: Waters Xbridge BEH C₁₈, 100×30 mm, 10 μm; mobile phase: [water (10 mM NH₄HCO₃)−ACN]; B: 23-53%, 10 min) gave the title compound as a white solid. Y=14%. ¹H NMR (400 MHz, DMSO-d₆) δ 8.66 (s, 1H), 7.76 (s, 1H), 4.59 (s, 2H), 4.13 (s, 3H), 3.75-3.50 (m, 1H), 3.28-3.23 (m, 1H), 2.88-2.70 (m, 1H), 2.25-2.15 (m, 1H), 2.14-2.00 (m, 1H), 1.75-1.50 (m, 4H), 1.49-1.40 (m, 1H), 1.31 (d, J=7 Hz, 6H), 1.25-1.16 (m, 1H), 0.50-0.35 (m, 2H), 0.30-0.15 (m, 2H). LC-MS (ESI): m/z: [M+H]⁺=373.3.

### Compound 4. 2-[2-(Cyclopropylmethyl)-7-isopropyl-4-oxo-pyrazolo[3,4-d]pyridazin-5-yl]-N-(5-fluoropyrimidin-4-yl)acetamide

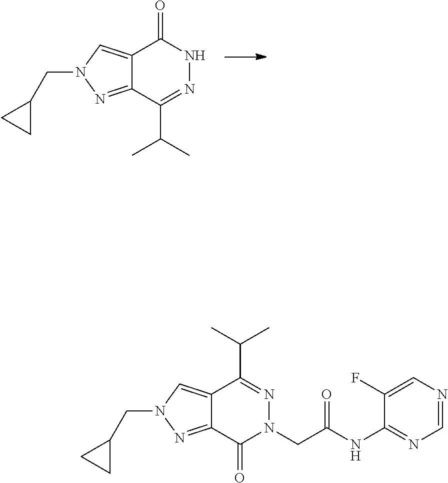 [SMILES: CC(C)c1n[nH]c(=O)c2cn(CC3CC3)nc12 ; CC(C)c1nn(CC(=O)Nc2ncncc2F)c(=O)c2nn(CC3CC3)cc12]

To a solution of 2-(cyclopropylmethyl)-7-isopropyl-5H-pyrazolo[3,4-d]pyridazin-4-one (Intermediate C4, 30 mg, 129 μmol) in DMF (1 mL) was added LiHMDS (1 M in THF, 258 μL, 0.258 mmol) at 0° C. and the mixture was stirred at 0° C. for 30 min. A solution of 2-chloro-N-(5-fluoropyrimidin-4-yl)acetamide (Intermediate B1, 36.7 mg, 194 μmol) in DMF (0.5 mL) was added at 0° C. and the resulting mixture was stirred at 30° C. for 12 h. The reaction mixture was quenched by aqueous saturated NH₄Cl solution (1 mL), filtered and the filtrate was concentrated under reduced pressure. Purification by prep-HPLC (column: Waters Xbridge BEH C₁₈, 100×30 mm, 10 μm; mobile phase: [water (10 mM NH₄HCO₃)−ACN]; B: 15-40%, 8 min) gave the title compound as a white solid. Y=47%. ¹H NMR (400 MHz, DMSO-d₆) δ 11.06 (s, 1H), 8.82-8.79 (m, 2H), 8.78 (s, 1H), 5.04 (s, 2H), 4.27 (d, J=7 Hz, 2H), 3.38-3.28 (m, 1H), 1.41-1.36 (m, 1H), 1.33 (d, J=7 Hz, 6H), 0.61-0.54 (m, 2H), 0.49-0.43 (m, 2H). LC-MS (ESI): m/z: [M+H]⁺=386.2.

### Compound 5. 2-(2-Ethyl-7-isopropyl-4-oxo-pyrazolo[3,4-d]pyridazin-5-yl)-N-(5-fluoropyrimidin-4-yl) acetamide

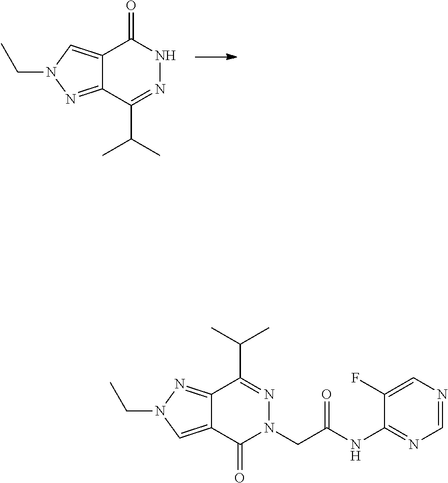 [SMILES: CCn1cc2c(=O)[nH]nc(C(C)C)c2n1 ; CCn1cc2c(=O)n(CC(=O)Nc3ncncc3F)nc(C(C)C)c2n1]

To a solution of 2-ethyl-7-isopropyl-5H-pyrazolo[3,4-d]pyridazin-4-one (Intermediate C5, 200 mg, 0.97 mmol) in DMF (1 mL) was added LiHMDS (1 M in THF, 1.94 mL, 1.94 mmol) at 0° C., the mixture was stirred at 0° C. for 30 min. To the mixture was added a solution of 2-chloro-N-(5-fluoropyrimidin-4-yl)acetamide (Intermediate B1, 276 mg, 1.45 mmol) in DMF (0.5 mL) at 0° C., and the resulting mixture was stirred at 30° C. for 3 h. The reaction mixture was quenched with saturated NH₄Cl (1 mL), filtered and the filtrate was concentrated under reduced pressure. Purification by prep-HPLC (column: Phenomenex Luna 80×30 mm, 3 μm; mobile phase: [water (0.04% HCl)−ACN]; B: 5-30%, 8 min) gave the title compound as a white solid. Y=30%. ¹H NMR (400 MHz, DMSO-d₆) δ 11.09 (s, 1H), 8.81 (d, J=2 Hz, 1H), 8.80 (d, J=2 Hz, 1H), 8.77 (s, 1H), 5.03 (s, 2H), 4.43 (q, J=7 Hz, 2H), 3.33-3.24 (m, 1H), 1.49 (t, J=7 Hz, 3H), 1.32 (d, J=7 Hz, 6H). LC-MS (ESI): m/z: [M+H]⁺=360.1.

### Compound 6. 2-(2-Ethyl-7-isopropyl-4-oxo-pyrazolo[3,4-d]pyridazin-5-yl)-N-[(3R)-1-methyl-3-piperidyl]acetamide

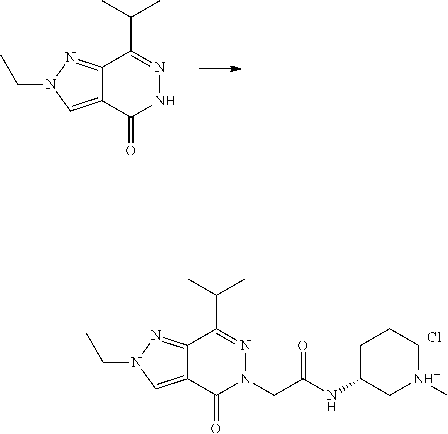 [SMILES: CCn1cc2c(=O)[nH]nc(C(C)C)c2n1 ; CCn1cc2c(=O)n(CC(=O)N[C@@H]3CCC[NH+](C)C3)nc(C(C)C)c2n1 ; Cl]

A mixture of 2-chloro-N-[(3R)-1-methyl-3-piperidyl]acetamide (Intermediate A3, 97.1 mg, 509 μmol), Cs₂CO₃ (442 mg, 1.36 mmol) and 2-ethyl-7-isopropyl-5H-pyrazolo[3,4-d]pyridazin-4-one (Intermediate C5, 70 mg, 339 μmol) in DMF (1 mL) was stirred at 60° C. for 8 h. The reaction mixture was diluted with water (1 mL) and extracted with EtOAc (3×1 mL). The combined organic layers were washed with brine (3×1 mL), dried over Na₂SO₄, filtered and concentrated. Purification by prep-HPLC (column: Phenomenex Luna C₁₈, 75×30 mm, 3 μm; mobile phase: [water (0.04% HCl)−ACN]; B: 1-25%, 8 min) gave the title compound as a white solid. Y=25%. ¹H NMR (400 MHz, DMSO-d₆) δ 10.57-10.36 (m, 1H), 8.70 (s, 1H), 8.68-8.26 (m, 1H), 4.69-4.56 (m, 2H), 4.47-4.38 (m, 2H), 4.05-3.96 (m, 1H), 3.40-3.08 (m, 3H), 2.90-2.70 (m, 4H), 2.70-2.58 (m, 1H), 1.92-1.61 (m, 3H), 1.48 (t, J=7 Hz, 3H), 1.44-1.34 (m, 1H), 1.31 (d, J=7 Hz, 6H). LC-MS (ESI): m/z: [M+H]+=361.2.

### Compound 7. N-[Cis-1,2,3,5,6,7,8,8a-octahydroindolizin-8-yl]-2-(2-ethyl-7-isopropyl-4-oxo-pyrazolo[3,4-d]pyridazin-5-yl) acetamide hydrochloride

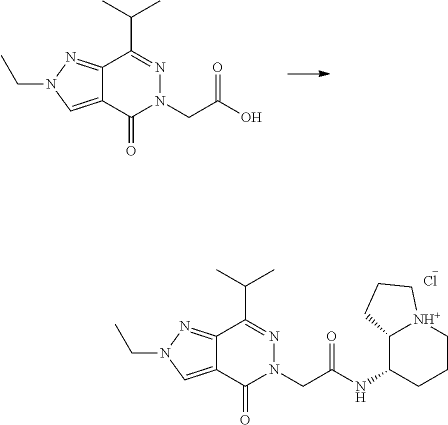

To a solution of 2-(2-ethyl-7-isopropyl-4-oxo-pyrazolo[3,4-d]pyridazin-5-yl)acetic acid (Intermediate C6, 60 mg, 227 μmol) in DMF (1 mL) was added HATU (86.3 mg, 227 μmol) at 0° C. The mixture was stirred at 0° C. for 10 min. Cis-1,2,3,5,6,7,8,8a-octahydroindolizin-8-amine hydrochloride (40.1 mg, 227 μmol) and DIPEA (119 μL, 681 μmol) were added at 0° C. The RM was stirred at 25° C. for 2 h. The reaction mixture was diluted with water (1 mL) and extracted with EtOAc (3×1 mL). The combined organic layers were washed with brine (3×1 mL), dried over Na₂SO₄, filtered and concentrated. Purification by Prep-HPLC (column: Phenomenex Luna C₁₈, 80×30 mm, 3 μm; mobile phase: [water (HCl)−ACN]; B: 1-30%, 8 min) gave the title compound as a white solid. Y=31%. ¹H NMR (400 MHz, DMSO-d₆) δ 10.50-10.39 (m, 1H), 8.74 (s, 1H), 8.28-8.16 (m, 1H), 4.64-4.60 (m, 2H), 4.45-4.39 (m, 2H), 3.86-3.79 (m, 1H), 3.56-3.33 (m, 2H), 3.31-3.21 (m, 1H), 3.14-2.90 (m, 2H), 2.89-2.73 (m, 1H), 2.17-2.07 (m, 1H), 1.94-1.81 (m, 4H), 1.80-1.58 (m, 2H), 1.51-1.40 (m, 4H), 1.30 (d, J=7 Hz, 6H). LC-MS (ESI): m/z: [M+H]⁺=387.2.

### Compound 8. 2-(2-Ethyl-7-isopropyl-4-oxo-pyrazolo[3,4-d]pyridazin-5-yl)-N-(5-fluoropyrimidin-2-yl)acetamide

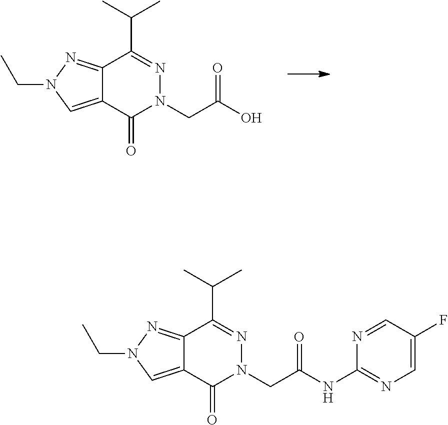 [SMILES: CCn1cc2c(=O)n(CC(=O)O)nc(C(C)C)c2n1 ; CCn1cc2c(=O)n(CC(=O)Nc3ncc(F)cn3)nc(C(C)C)c2n1]

To a solution of 5-fluoropyrimidin-2-amine (Intermediate B2, 257 mg, 2.27 mmol) in ACN (5 mL) was added 2-(2-ethyl-7-isopropyl-4-oxo-pyrazolo [3,4-d]pyridazin-5-yl)acetic acid (Intermediate C6, 400 mg, 1.51 mmol), COMU (843 mg, 1.97 mmol) and N-methylmorpholine (166 μL, 1.51 mmol) at 25° C. The mixture was stirred at 50° C. for 8 h. The reaction mixture was concentrated under reduced pressure. Purification by prep-HPLC (column: Waters Xbridge BEH C18, 250×50 mm, 10 μm; mobile phase: [water (10 mM NH₄HCO₃)−ACN]; B: 10-50%, 10 min) gave the title compound as a white solid. Y=50%. ¹H NMR (400 MHz, DMSO-d₆) δ 11.03 (s, 1H), 8.81-8.73 (m, 3H), 5.01 (s, 2H), 4.43 (q, J=8 Hz, 2H), 3.30-3.25 (m, 1H), 1.49 (t, J=8 Hz, 3H), 1.32 (d, J=7 Hz, 6H). LC-MS (ESI): m/z: [M+H]⁺=360.2.

### Compound 9. N-[(3R)-1-Cyclopropyl-3-piperidyl]-2-(4-isopropyl-2-methyl-7-oxo-pyrazolo[3,4-d]pyridazin-6-yl)acetamide

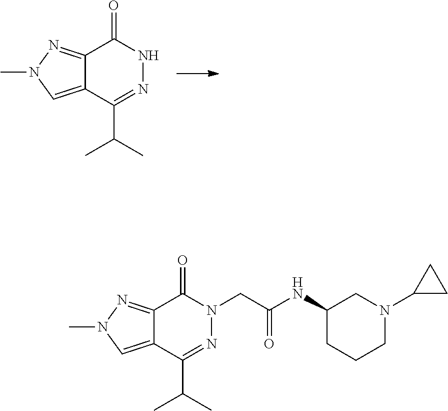 [SMILES: CC(C)c1n[nH]c(=O)c2nn(C)cc12 ; CC(C)c1nn(CC(=O)N[C@@H]2CCCN(C3CC3)C2)c(=O)c2nn(C)cc12]

To a solution of 4-isopropyl-2-methyl-6H-pyrazolo[3,4-d]pyridazin-7-one (Intermediate C7, 150 mg, 780 μmol) in DMF (3 mL) was added 2-chloro-N-[(3R)-1-cyclopropyl-3-piperidyl]acetamide (Intermediate A2,338 mg, 1.56 mmol) and Cs₂CO₃ (509 mg, 1.56 mmol) at 25° C. The RM was stirred at 50° C. for 17 h under N₂, then filtered and the filtrate was concentrated. Purification by prep-HPLC (column: Waters Xbridge Prep OBD C18, 150×40 mm, 10 μm; mobile phase: [water (10 mM NH₄HCO₃)−ACN]; B: 15-45%, 8 min) gave the title compound as a white solid. Y=18%. ¹H NMR (400 MHz, DMSO-d₆) δ 8.67 (s, 1H), 7.79 (d, J=8 Hz, 1H), 4.60 (s, 2H), 4.14 (s, 3H), 3.71-3.57 (m, 1H), 3.09-3.02 (m, 1H), 2.82 (d, J=9 Hz, 1H), 2.72-2.61 (m, 1H), 2.18 (t, J=9 Hz, 1H), 2.04 (t, J=9 Hz, 1H), 1.70-1.61 (m, 1H), 1.61-1.54 (m, 2H), 1.44-1.31 (m, 1H), 1.30-1.20 (m, 7H), 0.41-0.34 (m, 2H), 0.26-0.16 (m, 2H). LCMS (ESI): m/z: [M+H]⁺=373.1.

### Compound 10. 2-(2-Chloro-7-isopropyl-4-oxo-thieno [2,3-d]pyridazin-5-yl)-N-[(3R)-1-cyclopropyl-3-piperidyl]acetamide

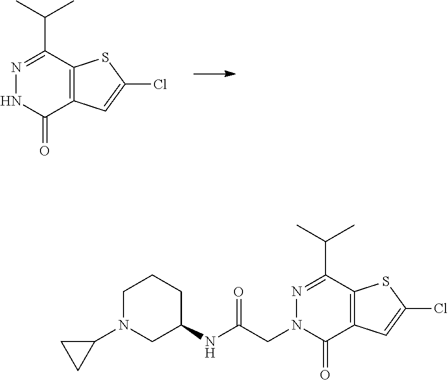 [SMILES: CC(C)c1n[nH]c(=O)c2cc(Cl)sc12 ; CC(C)c1nn(CC(=O)N[C@@H]2CCCN(C3CC3)C2)c(=O)c2cc(Cl)sc12]

To a solution of 2-chloro-7-isopropyl-5H-thieno[2,3-d]pyridazin-4-one (Intermediate C8, 70 mg, 306 μmol) in DMF (2 mL) was added 2-chloro-N-[(3R)-1-cyclopropyl-3-piperidyl]acetamide (Intermediate A2, 133 mg, 612 μmol) and Cs₂CO₃ (299 mg, 918 μmol). The RM was stirred at 25° C. for 1 h. Purification by prep-HPLC (column: Waters Xbridge BEH C18, 100×30 mm, 10 μm; mobile phase: [water (10 mM NH₄HCO₃)−ACN]; B: 40-60%, 8 min) gave the title compound as a white solid. Y=48%. 1H NMR (400 MHz, DMSO-d₆) δ 7.91 (d, J=8 Hz, 1H), 7.74 (s, 1H), 4.69 (s, 2H), 3.55-3.71 (m, 1H), 3.09-3.01 (m, 1H), 2.86-2.83 (m, 1H), 2.63-2.75 (m, 1H), 2.18 (t, J=10 Hz, 1H), 2.04 (t, J=10 Hz, 1H), 1.53-1.74 (m, 3H), 1.45-1.20 (m, 8H), 0.32-0.47 (m, 2H), 0.15-0.29 (m, 2H). LCMS (ESI): m/z: [M+H]⁺=409.2.

### Compound 11. 2-(2-Chloro-4-isopropyl-7-oxo-thieno[2,3-d]pyridazin-6-yl)-N-[(3R)-1-cyclopropyl-3-piperidyl]acetamide

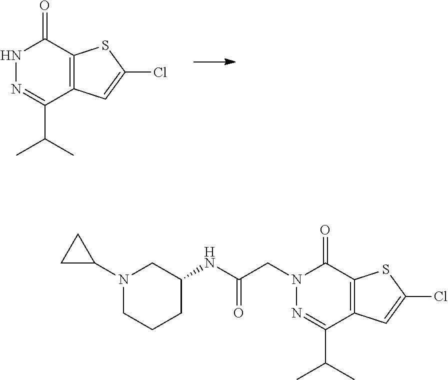 [SMILES: CC(C)c1n[nH]c(=O)c2sc(Cl)cc12 ; CC(C)c1nn(CC(=O)N[C@@H]2CCCN(C3CC3)C2)c(=O)c2sc(Cl)cc12]

To a solution of 2-chloro-4-isopropyl-6H-thieno[2,3-d]pyridazin-7-one (Intermediate C8, 50 mg, 219 μmol) in DMF (1 mL) at 25° C. was added 2-chloro-N-[(3R)-1-cyclopropyl-3-piperidyl]acetamide (Intermediate A2, 71.1 mg, 328 μmol) and Cs₂CO₃ (107 mg, 328 μmol). The RM was stirred at 50° C. for 10 h. The reaction mixture was filtered and the filtrate concentrated in vacuo. Purification by prep-HPLC (column: Waters Xbridge BEH C18 100×25 mm, 5 μm; mobile phase: [water (10 mM NH₄HCO₃)−ACN]; B: 25-65%, 10 min) gave the title compound as a white solid. Y=51%. ¹H NMR (400 MHz, DMSO-d₆) δ 7.98-7.89 (m, 2H), 4.70 (s, 2H), 3.68-3.57 (m, 1H), 3.32-3.27 (m, 1H), 2.84 (d, J=8 Hz, 1H), 2.73-2.67 (m, 1H), 2.18 (t, J=10 Hz, 1H), 2.08-1.99 (m, 1H), 1.74-1.54 (m, 3H), 1.45-1.33 (m, 1H), 1.30-1.15 (m, 7H), 0.43-0.33 (m, 2H), 0.29-0.17 (m, 2H). LCMS (ESI): m/z: [M+H]*=409.2.

### Compound 12. 2-(2-Ethyl-7-isopropyl-4-oxo-thieno[2,3-d]pyridazin-5-yl)-N-(5-fluoropyrimidin-2-yl)acetamide

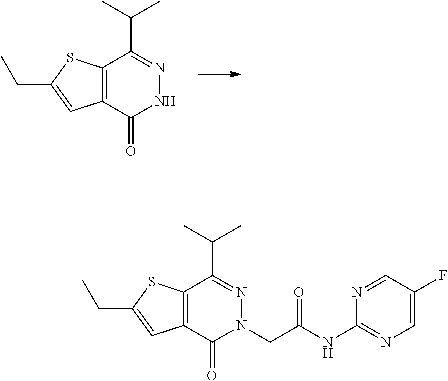 [SMILES: CCc1cc2c(=O)[nH]nc(C(C)C)c2s1 ; CCc1cc2c(=O)n(CC(=O)Nc3ncc(F)cn3)nc(C(C)C)c2s1]

To a solution of 2-ethyl-7-isopropyl-5H-thieno[2,3-d]pyridazin-4-one (Intermediate C10, 50 mg, 225 μmol) in DMF (2 mL) at 25° C. was added 2-chloro-N-(5-fluoropyrimidin-2-yl)acetamide (Intermediate B2, 85.3 mg, 450 μmol) and Cs₂CO₃ (220 mg, 675 μmol). The RM was stirred at 80° C. for 2 h then concentrated in vacuo. Purification by prep-HPLC (column: Waters Xbridge BEH C18 100×30 mm, 10 μm; mobile phase: [water (NH₄HCO₃)−ACN]; B: 30-60%, 10 min) gave the title compound as a white solid. Y=59%. ¹H NMR (400 MHz, DMSO-d₆) δ 11.10 (s, 1H), 8.77 (s, 2H), 7.42 (s, 1H), 5.10 (s, 2H), 3.15-3.03 (m, 1H), 2.99 (q, J=7 Hz, 2H), 1.35-1.28 (m, 9H). LCMS (ESI): m/z: [M+H]⁺=376.2.

### Compound 13. N-[(3R)-1-Cyclopropyl-3-piperidyl]-2-(7-isopropenyl-1-methyl-4-oxo-imidazo[4,5-d]pyridazin-5-yl)acetamide

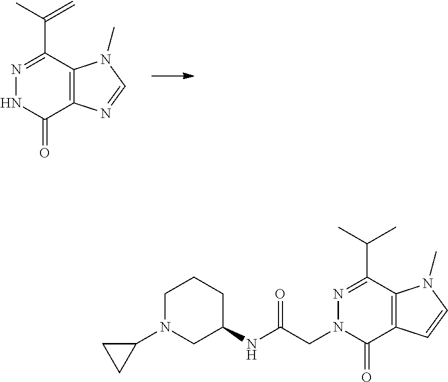 [SMILES: C=C(C)c1n[nH]c(=O)c2ncn(C)c12 ; CC(C)c1nn(CC(=O)N[C@@H]2CCCN(C3CC3)C2)c(=O)c2ccn(C)c12]

To a solution of 4-isopropenyl-3-methyl-6H-imidazo[4,5-d]pyridazin-7-one (Intermediate C11, 60 mg, 315 μmol) in THE (2 mL) at 0° C. was added NaH (60% in mineral oil, 25.2 mg, 631 μmol) and 2-chloro-N-[(3R)-1-cyclopropyl-3-piperidyl]acetamide (Intermediate A2, 137 mg, 631 μmol). The RM was stirred at 55° C. for 2 h. Water (1 mL) was added dropwise and the resulting mixture was stirred for 5 min and extracted with ethyl acetate (3×2 mL). The combined organic phase was washed with brine (2 mL), dried with anhydrous Na₂SO₄, filtered and concentrated under vacuum. Purification by prep-HPLC (column: Waters Xbridge BEH C18 100×25 mm, 5 μm; mobile phase: [water (10 mM NH₄HCO₃)−ACN]; B: 5-45%, 10 min) gave the title compound as a white solid. Y=53%. ¹H NMR (400 MHz, DMSO-d₆) δ 8.30 (s, 1H), 7.88 (d, J=8 Hz, 1H), 5.57 (s, 1H), 5.24 (s, 1H), 4.71 (s, 2H), 3.82 (s, 3H), 3.69-3.56 (m, 1H), 2.84 (d, J=8 Hz, 1H), 2.74-2.64 (m, 1H), 2.25-2.15 (m, 1H), 2.12 (s, 3H), 2.08-1.99 (m, 1H), 1.74-1.53 (m, 3H), 1.47-1.13 (m, 2H), 0.45-0.30 (m, 2H), 0.29-0.19 (m, 2H). LCMS (ESI): m/z: [M+H]⁺=371.3.

### Compound 14. N-[(3R)-1-Cyclopropyl-3-piperidyl]-2-(7-isopropyl-1-methyl-4-oxo-imidazo[4,5-d]pyridazin-5-yl)acetamide

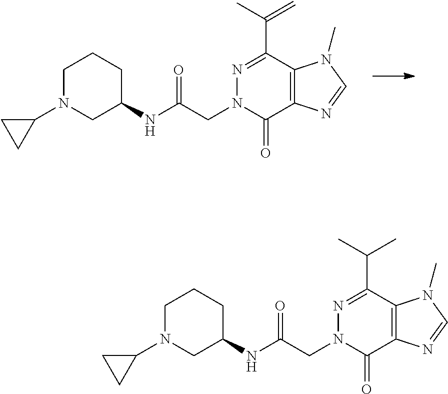 [SMILES: C=C(C)c1nn(CC(=O)N[C@@H]2CCCN(C3CC3)C2)c(=O)c2ncn(C)c12 ; CC(C)c1nn(CC(=O)N[C@@H]2CCCN(C3CC3)C2)c(=O)c2ncn(C)c12]

To a solution of N-[(3R)-1-cyclopropyl-3-piperidyl]-2-(7-isopropenyl-1-methyl-4-oxo-imidazo [4,5-d]pyridazin-5-yl)acetamide (Compound 13, 40 mg, 108 μmol) in MeOH (1 mL) at 25° C. was added 10% Pd on carbon (50% in water, 40 mg) under N₂. The suspension was degassed under vacuum and purged with H₂ several times. The mixture was stirred under H₂ (15 psi) at 25° C. for 30 min. The reaction mixture was filtered and the filtrate concentrated. Purification by prep-HPLC (column: Waters Xbridge BEH C18 100×25 mm, 5 μm; mobile phase: [water (10 mM NH₄HCO₃)−ACN]; B: 20-50%, 10 min) gave the title compound as a white solid. Y=40%. ¹H NMR (400 MHz, DMSO-d₆) δ 8.23 (s, 1H), 7.80 (d, J=8 Hz, 1H), 4.67 (s, 2H), 4.02 (s, 3H), 3.68-3.58 (m, 1H), 3.55-3.45 (m, 1H), 2.83 (d, J=9 Hz, 1H), 2.72-2.63 (m, 1H), 2.25-1.99 (m, 2H), 1.71-1.52 (m, 3H), 1.46-1.32 (m, 1H), 1.30-1.16 (m, 7H), 0.42-0.34 (m, 2H), 0.26-0.18 (m, 2H). LCMS (ESI): m/z: [M+H]⁺=373.3.

### Compound 15. 2-(2,7-Diisopropyl-4-oxo-pyrazolo[3,4-d]pyridazin-5-yl)-N-(5-fluoropyrimidin-2-yl)acetamide

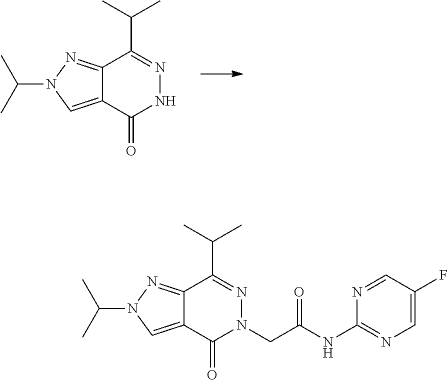 [SMILES: CC(C)c1n[nH]c(=O)c2cn(C(C)C)nc12 ; CC(C)c1nn(CC(=O)Nc2ncc(F)cn2)c(=O)c2cn(C(C)C)nc12]

To a solution of 2,7-diisopropyl-5H-pyrazolo[3,4-d]pyridazin-4-one (100 mg, 454 μmol) in DMF (0.5 mL) at 0° C. was added LiHMDS (1 M in THF, 0.91 mL, 0.91 mmol). After 30 mins, a solution of 2-chloro-N-(5-fluoropyrimidin-2-yl)acetamide (Intermediate B2, 129 mg, 681 μmol) in DMF (0.5 mL) was added. The RM was stirred at 25° C. for 8 h. The reaction mixture was quenched with aqueous NH₄Cl (1 mL), filtered and the filtrate was concentrated under reduced pressure. Purification by prep-HPLC (column: Waters Xbridge BEH C18 100×25 mm, 5 μm; mobile phase: [water (NH₄HCO₃)−ACN]; B: 20-50%, 10 min) gave the title compound as a white solid. Y=24%. ¹H NMR (400 MHz, DMSO-d₆) δ 11.03 (s, 1H), 8.81 (s, 1H), 8.77 (s, 2H), 5.02 (s, 2H), 4.88-4.74 (m, 1H), 3.31-3.26 (m, 1H), 1.54 (d, J=7 Hz, 6H), 1.32 (d, J=7 Hz, 6H). LC-MS (ESI): m/z: [M+H]⁺=374.0.

### Compound 16. 2-(2,7-diisopropyl-4-oxo-pyrazolo[3,4-d]pyridazin-5-yl)-N-pyrimidin-2-yl-acetamide

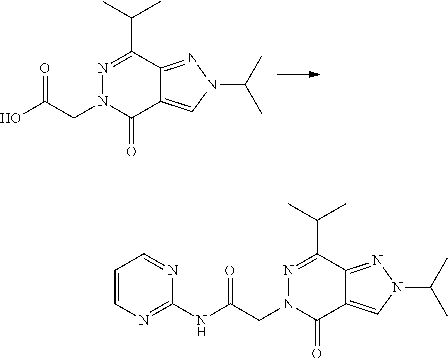 [SMILES: CC(C)c1nn(CC(=O)O)c(=O)c2cn(C(C)C)nc12 ; CC(C)c1nn(CC(=O)Nc2ncccn2)c(=O)c2cn(C(C)C)nc12]

To a solution of pyrimidin-2-amine (25.6 mg, 269 μmol) in ACN (1 mL) at 25° C. was added 2-(2,7-diisopropyl-4-oxo-pyrazolo[3,4-d]pyridazin-5-yl)acetic acid (Intermediate C12, 50 mg, 180 μmol), COMU (115 mg, 269 μmol) and N-methylmorpholine (20 μL, 180 μmol,). The resulting mixture was stirred under N₂ at 50° C. for 12 h. The reaction mixture was concentrated under reduced pressure. Purification by prep-HPLC (column: Waters Xbridge BEH C18 100×30 mm, 10 μm; mobile phase: [water (NH₄HCO₃)−ACN]; B: 20-50%, 8 min) gave the title compound as a white solid. Y=25%. ¹H NMR (400 MHz, DMSO-d₆) δ 10.87 (s, 1H), 8.80 (s, 1H), 8.67 (d, J=5 Hz, 2H), 7.20 (t, J=5 Hz, 1H), 5.08 (s, 2H), 4.86-4.77 (m, 1H), 3.40-3.28 (m, 1H), 1.54 (d, J=7 Hz, 6H), 1.32 (d, J=7 Hz, 6H). LC-MS (ESI): m/z: [M+H]⁺=356.2.

### Compound 17. 2-(2-Cyclopropyl-7-isopropyl-4-oxo-pyrazolo[3, 4-d]pyridazin-5-yl)-N-(5-fluoropyrimidin-2-yl) acetamide

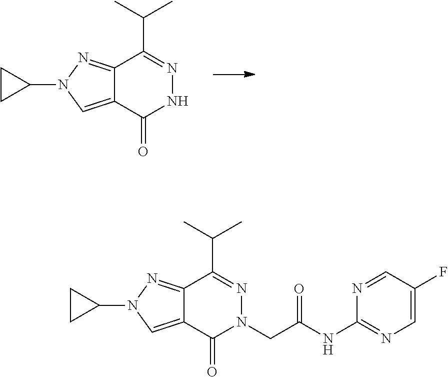 [SMILES: CC(C)c1n[nH]c(=O)c2cn(C3CC3)nc12 ; CC(C)c1nn(CC(=O)Nc2ncc(F)cn2)c(=O)c2cn(C3CC3)nc12]

To a mixture of 2-cyclopropyl-7-isopropyl-5H-pyrazolo[3,4-d]pyridazin-4-one (Intermediate C13, 0.15 g, 687 μmol) in DMF (3 mL) at 25° C. was added LiHMDS (1 M in THF, 1.37 mL, 1.37 mmol) and 2-chloro-N-(5-fluoropyrimidin-2-yl)acetamide (Intermediate B2, 130 mg, 687 μmol). The mixture was stirred at 25° C. for 2 h. Purification by prep-HPLC (column: Waters Xbridge BEH C₁₈ 100×30 mm, 10 μm; mobile phase: [water (NH₄HCO₃)−ACN]; B: 20-50%,8 min) gave the title compound as a white solid. Y=10%. ¹H NMR (400 MHz, DMSO-d₆) δ 11.02 (s, 1H), 8.82 (s, 1H), 8.76 (s, 2H), 5.01 (s, 2H), 4.15-4.05 (m, 1H), 3.30-3.23 (m, 1H), 1.30 (d, J=7 Hz, 6H), 1.28-1.25 (m, 2H), 1.16-1.10 (m, 2H). LCMS (ESI): m/z: [M+H]⁺=372.0.

### Compound 18. 2-(7-Cyclopropyl-1-isopropyl-4-oxo-pyrido[3,4-d]pyridazin-3-yl)-N-(2-oxaspiro[3.3]heptan-6-yl)acetamide

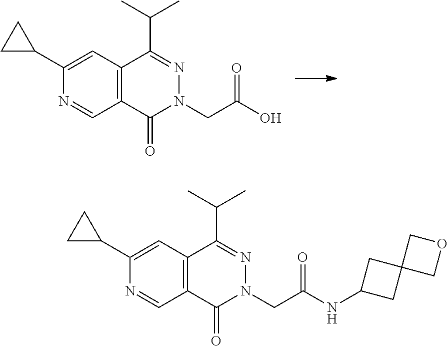 [SMILES: CC(C)c1nn(CC(=O)O)c(=O)c2cnc(C3CC3)cc12 ; CC(C)c1nn(CC(=O)NC2CC3(COC3)C2)c(=O)c2cnc(C3CC3)cc12]

To a solution of 2-(7-cyclopropyl-1-isopropyl-4-oxo-pyrido [3,4-d]pyridazin-3-yl)acetic acid (Intermediate C14, 53 mg, 184 μmol) in DMF (1 mL) at 0° C. was added slowly HATU (105 mg, 277 μmol). The mixture was stirred at 0° C. for 30 mins. 2-Oxaspiro[3.3]heptan-6-amine hydrochloride (41.4 mg, 277 μmol) and DIPEA (112 μL, 646 μmol) were added at 0° C. The resulting mixture was stirred at 25° C. for 2 h. The reaction mixture was diluted with water (2 mL) and extracted with DCM (3×2 mL). The combined organic layers were washed with brine (2×2 mL), dried over Na₂SO₄, filtered and concentrated under reduced pressure. Purification by prep-HPLC (column: Waters Xbridge BEH C18, 100×30 mm, 10 μm; mobile phase: [water (NH₄HCO₃)−ACN]; B: 20-50%, 8 min) gave the title compound as a white solid. Y=71%. ¹H NMR (400 MHz, DMSO-d₆) δ 9.29 (s, 1H), 8.24 (d, J=7 Hz, 1H), 7.87 (s, 1H), 4.62 (s, 2H), 4.57 (s, 2H), 4.46 (s, 2H), 4.05-3.97 (m, 1H), 3.58-3.54 (m, 1H), 2.44-2.39 (m, 2H), 2.15-2.01 (m, 3H), 1.24 (d, J=7 Hz, 6H), 1.13-1.07 (m, 4H). LCMS (ESI): m/z: [M+H]⁺=383.2.

### Compound 19. 2-(7-Cyclopropyl-1-isopropyl-4-oxo-pyrido[3,4-d]pyridazin-3-yl)-N-(5-methyl-1,2,4-thiadiazol-3-yl)acetamide

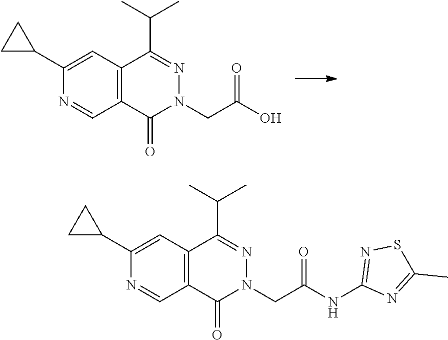 [SMILES: CC(C)c1nn(CC(=O)O)c(=O)c2cnc(C3CC3)cc12 ; Cc1nc(NC(=O)Cn2nc(C(C)C)c3cc(C4CC4)ncc3c2=O)ns1]

To a solution of 2-(7-cyclopropyl-1-isopropyl-4-oxo-pyrido[3,4-d]pyridazin-3-yl)acetic acid (Intermediate C14, 50 mg, 174 μmol) in ACN (0.5 mL) at 25° C. was added COMU (112 mg, 261 μmol), 5-methyl-1,2,4-thiadiazol-3-amine (39.6 mg, 344 μmol) and N-methylmorpholine (19.1 μL, 174 μmol). The resulting mixture was stirred at 50° C. for 12 h then concentrated in vacuo. Purification by prep-HPLC (column: Waters Xbridge BEH C18, 100×30 mm, 10 μm; mobile phase: [water (10 mM NH₄HCO₃)−ACN]; B: 25-50%, 8 min) gave the title compound as a white solid. Y=60% yield. ¹H NMR (400 MHz, DMSO-d₆) δ 11.51 (s, 1H), 9.31 (s, 1H), 7.90 (s, 1H), 5.02 (s, 2H), 3.61-3.57 (m, 1H), 2.76 (s, 3H), 2.45-2.40 (m, 1H), 1.27 (d, J=7 Hz, 6H), 1.16-1.08 (m, 4H). LCMS (ESI): m/z: [M+H]⁺=385.2.

### Compound 20. 2-(2,7-Diethyl-4-oxo-pyrazolo[3,4-d]pyridazin-5-yl)-N-(5-fluoropyrimidin-2-yl)acetamide

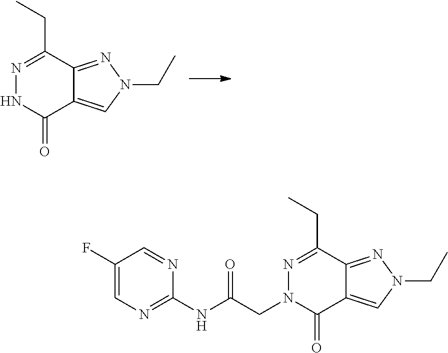 [SMILES: CCc1n[nH]c(=O)c2cn(CC)nc12 ; CCc1nn(CC(=O)Nc2ncc(F)cn2)c(=O)c2cn(CC)nc12]

To a solution of 2,7-diethyl-5H-pyrazolo[3,4-d]pyridazin-4-one (for synthesis see Intermediate C15) (70 mg, 364 μmol) in DMF (1 mL) at 0° C. was added LiHMDS (1 M in THF, 728 μL). The RM was stirred at 0° C. for 30 min. A solution of 2-chloro-N-(5-fluoropyrimidin-2-yl)acetamide (Intermediate B2, 104 mg, 546 μmol) in DMF (0.2 mL) at 0° C. was added. The RM was stirred at 25° C. for 8 h. The reaction mixture was quenched with aqueous NH₄Cl (1 mL), filtered and the filtrate was concentrated under reduced pressure. Purification by prep-HPLC (column: Waters Xbridge BEH C18 100×30 mm, 10 μm; mobile phase: [water (10 mM NH₄HCO₃)−ACN]; B: 10-40%, 8 min) gave the title compound as a white solid. Y=32%. ¹H NMR (400 MHz, DMSO-d₆) δ 11.03 (s, 1H), 8.77 (s, 3H), 5.02 (s, 2H), 4.43 (q, J=7 Hz, 2H), 2.84 (q, J=7 Hz, 2H), 1.49 (t, J=7 Hz, 3H), 1.27 (t, J=7 Hz, 3H). LC-MS (ESI): m/z: [M+H]⁺=346.2.

### Compound 21. 2-(2,7-Diethyl-4-oxo-pyrazolo[3,4-d]pyridazin-5-yl)-N-pyrimidin-2-yl-acetamide

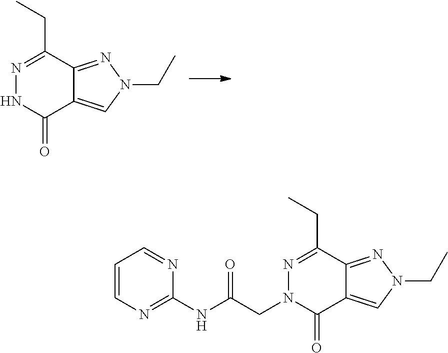 [SMILES: CCc1n[nH]c(=O)c2cn(CC)nc12 ; CCc1nn(CC(=O)Nc2ncccn2)c(=O)c2cn(CC)nc12]

To a solution of 2,7-diethyl-5H-pyrazolo[3,4-d]pyridazin-4-one (for synthesis see Intermediate C15) (100 mg, 0.52 mmol) in DMF (1 mL) at 0° C. was added LiHMDS (1 M in THF, 1.04 mL, 1.04 mmol). After stirring for 20 mins, a solution of 2-chloro-N-pyrimidin-2-yl-acetamide (Intermediate B3, 134 mg, 780 μmol) in DMF (0.5 mL) was added dropwise. The RM was stirred at 25° C. for 5 h. The reaction mixture was quenched with water (3 mL) at 0° C. The mixture was extracted with EtOAc (3×2 mL) and the combined organic layers washed with brine (2 mL), dried over Na₂SO₄, filtered and concentrated under reduced pressure. Purification by prep-HPLC (column: Waters Xbridge BEH C18, 100×30 mm, 10 μm; mobile phase: [water (10 mM NH₄HCO₃)−ACN]; B: 5-35%, 8 min) gave the title compound as a white solid. Y=28%. ¹H NMR (400 MHz, DMSO-d₆) δ 10.87 (s, 1H), 8.76 (s, 1H), 8.67 (d, J=5 Hz, 2H), 7.20 (t, J=5 Hz, 1H), 5.08 (s, 2H), 4.43 (q, J=7 Hz, 2H), 2.84 (q, J=7 Hz, 2H), 1.49 (t, J=7 Hz, 3H), 1.27 (t, J=7 Hz, 3H). LCMS (ESI): m/z: [M+H]⁺=328.2.

### Compound 22. 2-(2,7-Diethyl-4-oxo-pyrazolo[3,4-d]pyridazin-5-yl)-N-(2-oxaspiro[3.3]heptan-6-yl)acetamide

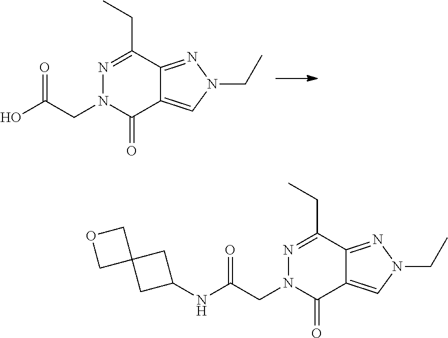 [SMILES: CCc1nn(CC(=O)O)c(=O)c2cn(CC)nc12 ; CCc1nn(CC(=O)NC2CC3(COC3)C2)c(=O)c2cn(CC)nc12]

To a solution of 2-(2,7-diethyl-4-oxo-pyrazolo[3,4-d]pyridazin-5-yl)acetic acid (Intermediate C15, 50 mg, 200 μmol) in DMF (0.5 mL) at 0° C. was added HATU (98.8 mg, 260 μmol). After stirring for 15 min, DIPEA (139 μL, 799 μmol) and 2-oxaspiro[3.3]heptan-6-amine hydrochloride (44.8 mg, 300 μmol) were added. The RM was stirred at 25° C. for 1 h under N₂. The reaction mixture was concentrated under reduced pressure. Purification by prep-HPLC (column: Waters Xbridge BEH C18, 100×30 mm, 10 μm; mobile phase: [water (10 mM NH₄HCO₃)−ACN]; B: 5-35%, 8 min) gave the title compound as a white solid. Y=39%. ¹H NMR (400 MHz, DMSO-d₆) δ 8.75 (s, 1H), 8.17 (d, J=8 Hz, 1H), 4.68-4.51 (m, 4H), 4.46 (s, 2H), 4.45-4.38 (m, 2H), 4.06-3.94 (m, 1H), 2.82 (q, J=7 Hz, 2H), 2.51-2.18 (m, 2H), 2.11-2.03 (m, 2H), 1.48 (t, J=7 Hz, 3H), 1.25 (t, J=7 Hz, 3H). LC-MS (ESI): m/z: [M+H]⁺=346.2.

### Compound 23. 2-(2-Ethyl-7-isopropyl-4-oxo-furo [2,3-d]pyridazin-5-yl)-N-(5-fluoropyrimidin-2-yl) acetamide

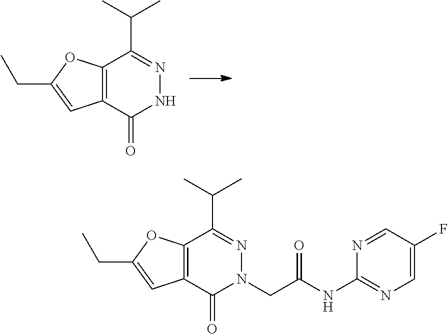 [SMILES: CCc1cc2c(=O)[nH]nc(C(C)C)c2o1 ; CCc1cc2c(=O)n(CC(=O)Nc3ncc(F)cn3)nc(C(C)C)c2o1]

To a solution of 2-ethyl-7-isopropyl-5H-furo[2,3-d]pyridazin-4-one (Intermediate C16, 50 mg, 242 μmol) in DMF (1 mL) at 25° C. was added Cs₂CO₃ (158 mg, 485 μmol) and 2-chloro-N-(5-fluoropyrimidin-2-yl)acetamide (Intermediate B2, 91.9 mg, 485 μmol). The mixture was stirred at 80° C. for 1 h. Purification by prep-HPLC (column: Waters Xbridge BEH C18 100×30 mm, 10 μm; mobile phase: [water (NH₄HCO₃)−ACN]; B: 30-60%, 10 min) gave the title compound as a white solid. Y=17%. ¹H NMR (400 MHz, DMSO-d₆) δ 11.07 (br. s, 1H), 8.76 (s, 2H), 6.81 (s, 1H), 5.10 (s, 2H), 3.27-3.18 (m, 1H), 2.85 (q, J=7 Hz, 2H), 1.36-1.20 (m, 9H). LCMS (ESI): m/z: [M+H]⁺=360.0.

### Compound 24. 2-(2-Ethyl-4-isopropyl-7-oxo-thieno[2,3-d]pyridazin-6-yl)-N-(5-fluoropyrimidin-2-yl) acetamide

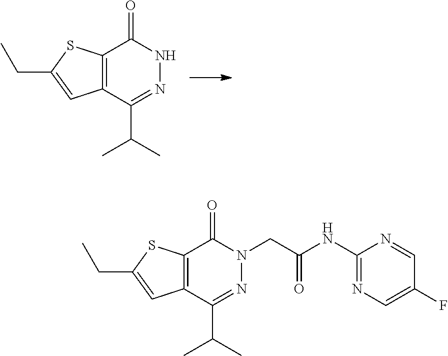 [SMILES: CCc1cc2c(C(C)C)n[nH]c(=O)c2s1 ; CCc1cc2c(C(C)C)nn(CC(=O)Nc3ncc(F)cn3)c(=O)c2s1]

To a solution of 2-ethyl-4-isopropyl-6H-thieno[2,3-d]pyridazin-7-one (Intermediate C17, 100 mg, 450 μmol) in DMF (2 mL) at 25° C. was added Cs₂CO₃ (440 mg, 1.35 mmol) and 2-chloro-N-(5-fluoropyrimidin-2-yl)acetamide (Intermediate B2, 128 mg, 675 μmol). The RM was stirred at 80° C. for 2 h. The mixture was diluted with water (3 mL) and extracted with ethyl acetate (3×3 mL). The combined organic layers were washed with brine (3 mL), dried over anhydrous Na₂SO₄, filtered and concentrated under reduced pressure. Purification by prep-HPLC (column: Waters Xbridge BEH C₁₈ 100×30 mm, 10 μm; mobile phase: [water (10 mM NH₄HCO₃)−ACN]; B: 30-60%, 10 min) gave the title compound as a white solid. Y=13%. ¹H NMR (400 MHz, DMSO-d₆) δ 11.11 (s, 1H), 8.77 (s, 2H), 7.47 (s, 1H), 5.11 (s, 2H), 3.32-3.28 (m, 1H), 3.01 (q, J=7 Hz, 2H), 1.33 (t, J=7 Hz, 3H), 1.26 (d, J=7 Hz, 6H). LCMS (ESI): m/z: [M+H]⁺=376.1.

### Compound 25. 2-(2-Cyclopropyl-8-isopropyl-5-oxo-pyrido[2,3-d]pyridazin-6-yl)-N-(2-oxaspiro[3.3]heptan-6-yl)acetamide

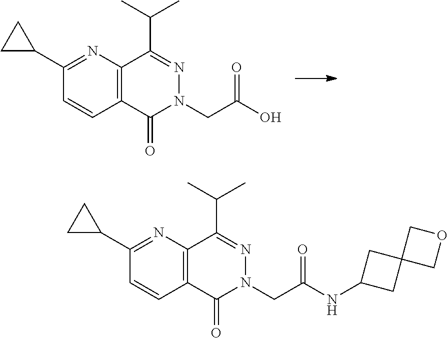 [SMILES: CC(C)c1nn(CC(=O)O)c(=O)c2ccc(C3CC3)nc12 ; CC(C)c1nn(CC(=O)NC2CC3(COC3)C2)c(=O)c2ccc(C3CC3)nc12]

To a solution of 2-(2-cyclopropyl-8-isopropyl-5-oxo-pyrido[2,3-d]pyridazin-6-yl)acetic acid (Intermediate C18, 50 mg, 174 μmol, Li salt) in DMF (1 mL) at 0° C. was added HATU (99 mg, 261 μmol). After stirring for 30 mins, 2-oxaspiro[3.3]heptan-6-amine hydrochloride (39.1 mg, 261 μmol) and DIPEA (106 μL, 609 μmol) were added at 0° C. The RM was stirred at 25° C. for 2 h, diluted with water (3 mL), and extracted with DCM (3×5 mL). The combined organic layers were washed with brine (2×10 mL), dried over Na₂SO₄, filtered and concentrated under reduced pressure. Purification by prep-HPLC (column: Waters Xbridge BEH C18, 100×30 mm, 10 μm; mobile phase: [water (10 mM NH₄HCO₃)−ACN]; B: 30-55%, 8 min) gave the title compound as a white solid. Y=45%. ¹H NMR (400 MHz, DMSO-d₆) δ 8.42 (d, J=8 Hz, 1H), 8.23 (d, J=7 Hz, 1H), 7.79 (d, J=8 Hz, 1H), 4.62 (s, 2H), 4.57 (s, 2H), 4.46 (s, 2H), 4.07-3.96 (m, 1H), 3.76-3.69 (m, 1H), 2.60-2.52 (m, 2H), 2.43-2.35 (m, 1H), 2.14-2.02 (m, 2H), 1.25 (d, J=7 Hz, 6H), 1.21-1.09 (m, 4H). LCMS (ESI): m/z: [M+H]⁺=383.2.

### Compound 26. 2-(2-Cyclopropyl-8-isopropyl-5-oxo-pyrido[2,3-d]pyridazin-6-yl)-N-(5-methyl-1,2,4-thiadiazol-3-yl)acetamide

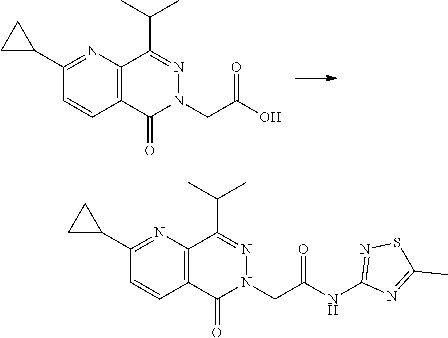 [SMILES: CC(C)c1nn(CC(=O)O)c(=O)c2ccc(C3CC3)nc12 ; Cc1nc(NC(=O)Cn2nc(C(C)C)c3nc(C4CC4)ccc3c2=O)ns1]

To a solution of 2-(2-cyclopropyl-8-isopropyl-5-oxo-pyrido[2,3-d]pyridazin-6-yl)acetic acid (Intermediate C18, 50 mg, 174 μmol, Li salt) in ACN (0.5 mL) at 25° C. was added COMU (112 mg, 261 μmol), 5-methyl-1,2,4-thiadiazol-3-amine (39.5 mg, 343 μmol) and N-methylmorpholine (19.1 μL, 174 μmol). The resulting mixture was stirred at 50° C. for 12 h, then concentrated under vacuum. Purification by prep-HPLC (column: Waters Xbridge BEH C18, 100×30 mm, 10 μm; mobile phase: [water (10 mM NH₄HCO₃)−ACN]; B: 30-60%, 8 min) gave the title compound as a white solid. Y=39%. ¹H NMR (400 MHz, DMSO-d₆) δ 11.47 (s, 1H), 8.41 (d, J=8 Hz, 1H), 7.78 (d, J=8 Hz, 1H), 5.00 (s, 2H), 3.78-3.69 (m, 1H), 2.74 (s, 3H), 2.41-2.34 (m, 1H), 1.25 (d, J=7 Hz, 6H), 1.19-1.09 (m, 4H). LCMS (ESI): m/z: [M+H]⁺=385.2.

### Compound 27. 2-(2-Cyclopropyl-8-isopropyl-5-oxo-pyrido [2,3-d]pyridazin-6-yl)-N-pyrimidin-2-yl-acetamide

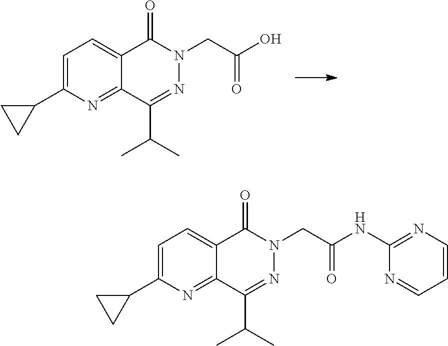 [SMILES: CC(C)c1nn(CC(=O)O)c(=O)c2ccc(C3CC3)nc12 ; CC(C)c1nn(CC(=O)Nc2ncccn2)c(=O)c2ccc(C3CC3)nc12]

To a solution of 2-(2-cyclopropyl-8-isopropyl-5-oxo-pyrido[2,3-d]pyridazin-6-yl)acetic acid (Intermediate C18, 50 mg, 174 μmol, Li salt) in ACN (0.5 mL) at 25° C. was added pyrimidin-2-amine (24.8 mg, 261 μmol), COMU (112 mg, 261 μmol) and N-methylmorpholine (19.1 μL, 174 μmol). The mixture was stirred at 80° C. for 2 h. The reaction mixture was diluted with water (1 mL) and extracted with EtOAc (3×1 mL). The combined organic layers were washed with brine (1 mL), dried over Na₂SO₄, filtered and concentrated. Purification by prep-HPLC (column: Phenomenex Luna 80×30 mm, 3 μm; mobile phase: [water (HCl)−ACN]; B: 15-65%, 8 min) gave the title compound as a white solid. Y=24%. ¹H NMR (400 MHz, DMSO-d₆) δ 10.95 (s, 1H), 8.67 (d, J=5 Hz, 2H), 8.44 (d, J=8 Hz, 1H), 7.81 (d, J=8 Hz, 1H), 7.21 (t, J=5 Hz, 1H), 5.15 (s, 2H), 3.77-3.73 (m, 1H), 2.41-2.39 (m, 1H), 1.27 (d, J=7 Hz, 6H), 1.20-1.12 (m, 4H). LCMS (ESI): m/z [M+H]⁺=365.2.

### Compound 28. 2-(2-Cyclopropyl-8-isopropyl-5-oxo-pyrido[2,3-d]pyridazin-6-yl)-N-(5-fluoropyrimidin-2-yl)acetamide

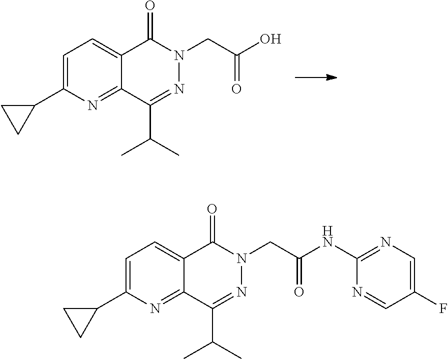 [SMILES: CC(C)c1nn(CC(=O)O)c(=O)c2ccc(C3CC3)nc12 ; CC(C)c1nn(CC(=O)Nc2ncc(F)cn2)c(=O)c2ccc(C3CC3)nc12]

Using 5-fluoro-2 amino pyrimidine and the same procedure as in Compound 27, the title compound was obtained as a white solid. Y=24%. ¹H NMR (400 MHz, DMSO-d₆) δ 11.10 (s, 1H), 8.77 (s, 2H), 8.44 (d, J=8 Hz, 1H), 7.81 (d, J=8 Hz, 1H), 5.09 (s, 2H), 3.79-3.71 (m, 1H), 2.41-2.39 (m, 1H), 1.27 (d, J=7 Hz, 6H), 1.20-1.10 (m, 4H). LCMS (ESI): m/z [M+H]⁺=383.2; RT=2.994 min.

### Compound 29. 2-(2-Bromo-4-isopropyl-7-oxo-thieno[2,3-d]pyridazin-6-yl)-N-[(3R)-1-cyclobutyl-3-piperidyl]acetamide

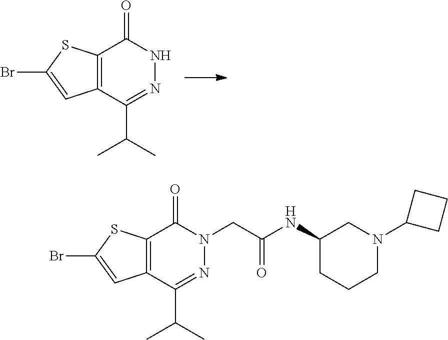

To a solution of 2-bromo-4-isopropyl-6H-thieno[2,3-d]pyridazin-7-one (Intermediate C19, 380 mg, 1.39 mmol) in THE (6 mL) at 25° C. was added Cs₂CO₃ (453 mg, 1.39 mmol) and 2-chloro-N-[(3R)-1-cyclobutyl-3-piperidyl]acetamide (Intermediate A4, 481 mg, 2.09 mmol). The RM was stirred at 60° C. for 3 h, then concentrated under reduced pressure. The residue was diluted with water (5 mL) and extracted with EtOAc (3×8 mL). The combined organic layers were washed with brine (2×10 mL), dried over Na₂SO₄, filtered and concentrated under reduced pressure to give a residue. Purification by prep-HPLC (column: Diacel Chiralpak AD (250 mm×30 mm, 10 μm); mobile phase: [0.1% NH₃H₂O in MeOH]; B: 45%, 10 min) gave the title compound as a racemate. Chiral separation by SFC (condition: NH₁₃H₂O) gave the title compound in 100% ee as a yellow solid. Y=51% yield. ¹H NMR (400 MHz, DMSO-d₆) δ 7.98 (s, 1H), 7.92 (d, J=8 Hz 1H), 4.70 (s, 2H), 3.69-3.65 (m, 1H), 3.31-3.29 (m, 1H), 2.65-2.58 (m, 2H), 2.55-2.45 (m, 1H), 2.00-1.90 (m, 2H), 1.82-1.53 (m, 8H), 1.43-1.39 (m, 2H), 1.24 (d, J=7 Hz, 6H). LCMS (ESI): m/z: [M+H]⁺=467.1.

### Compound 30. 2-(2-Cyano-4-isopropyl-7-oxo-thieno[2,3-d]pyridazin-6-yl)-N-[(3R)-1-cyclobutyl-3-piperidyl]acetamide

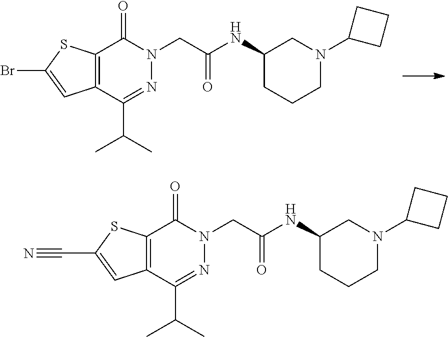

To a solution of 2-(2-bromo-4-isopropyl-7-oxo-thieno[2,3-d]pyridazin-6-yl)-N-[(3R)-1-cyclo butyl-3-piperidyl]acetamide (Compound 29, 200 mg, 428 μmol) in DMF (2 mL) at 25° C. was added Zn(CN)₂ (60 μL, 941 μmol), Pd₂(dba)₃ (39 mg, 43 μmol) and DPPF (47 mg, 86 μmol). The mixture was heated in a microwave reactor at 120° C. for 1 h under N₂. The RM was diluted with water (2 mL) and extracted with EtOAc (3×2 mL). The combined organic layers were washed with brine (3×1 mL), dried over Na₂SO₄, filtered and concentrated under reduced pressure. Purification by prep-HPLC (column: Waters Xbridge BEH C18, 100×30 mm, 10 μm; mobile phase: [water (10 mM NH₄HCO₃)−ACN]; B: 35-55%, 8 min) gave the title compound as a white solid. Y=10%. ¹H NMR (400 MHz, DMSO-d₆) δ 8.71 (s, 1H), 7.97 (d, J=8 Hz, 1H), 4.73 (s, 2H), 3.75-3.61 (m, 1H), 3.41-3.35 (m, 1H), 2.69-2.56 (m, 2H), 1.98-1.86 (m, 2H), 1.83-1.51 (m, 9H), 1.48-1.35 (m, 1H), 1.26 (d, J=7 Hz, 7H). LCMS (ESI): m/z: [M+H]⁺=414.1.

### Compound 31. N-[(3R)-1-Cyclobutyl-3-piperidyl]-2-[4-isopropyl-2-(methoxy methyl)-7-oxo-thieno [2,3-d]pyridazin-6-yl]acetamide hydrochloride

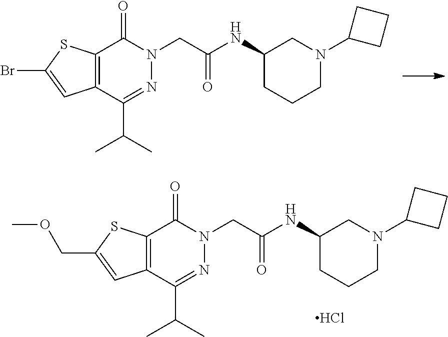

To a solution of 2-(2-bromo-4-isopropyl-7-oxo-thieno[2,3-d]pyridazin-6-yl)-N-[(3R)-1-cyclo butyl-3-piperidyl]acetamide (Compound 29, 20 mg, 42.8 μmol) in DMA (0.6 mL) at 25° C. were added potassium trifluoro(methoxymethyl)boranuide (13.0 mg, 85.6 μmol), dichloronickel 1,2-dimethoxyethane (94 μg, 4.28 μmol), 4-tert-butyl-2-(4-tert-butyl-2-pyridyl)pyridine (1.2 mg, 4.3 μmol), Na₂CO₃ (13.6 mg, 128 μmol) and bis[3,5-difluoro-2-[5-(trifluoromethyl)-2-pyridyl]phenyl]iridium2-(2-pyridyl)pyridinehexafluoro phosphate (2.2 mg, 2.1 μmol). The mixture was stirred at 25° C. for 3 h under a 26 W CFL light and under a N₂ atmosphere. The RM was diluted with water (5 mL) and extracted with EtOAc (3×8 mL). The combined organic layers were washed with brine (2×10 mL), dried over Na₂SO₄, filtered, and concentrated under reduced pressure. Purification by prep-TLC (SiO₂, EtOAc:MeOH=3:1) gave the title compound as a free base. This was treated with 1M HCl (2 mL) and lyophilized to give the title compound as a white solid. Y=6%. ¹H NMR (400 MHz, DMSO-d₆) δ 9.89-9.77 (br. s, 1H), 8.40 (d, J=8 Hz, 1H), 7.64 (s, 1H), 4.78 (s, 2H), 4.72 (s, 2H), 3.94-3.65 (m, 1H), 3.72-3.60 (m, 1H), 3.37 (s, 3H), 3.30-3.20 (m, 2H), 2.23-2.06 (m, 4H), 2.05-1.78 (m, 3H), 1.77-1.52 (m, 4H), 1.50-1.37 (m, 2H), 1.25 (d, J=7 Hz, 6H). LCMS (ESI): m/z: [M+H]⁺=433.2.

### Compound 32. 2-(2-Cyclopropyl-8-ethyl-5-oxo-pyrido[2,3-d]pyridazin-6-yl)-N-pyrimidin-2-yl-acetamide

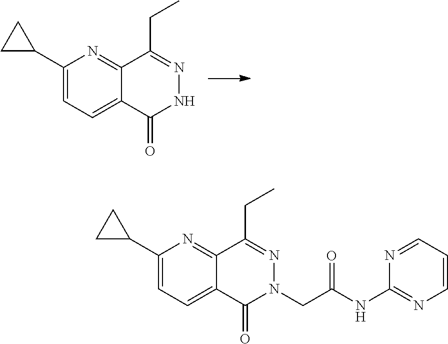 [SMILES: CCc1n[nH]c(=O)c2ccc(C3CC3)nc12 ; CCc1nn(CC(=O)Nc2ncccn2)c(=O)c2ccc(C3CC3)nc12]

To a solution of 2-cyclopropyl-8-ethyl-6H-pyrido[2,3-d]pyridazin-5-one (Intermediate C20, 50 mg, 232 μmol) in DMF (0.5 mL) at 0° C. was added NaH (60% in mineral oil, 32.5 mg, 813 μmol). The mixture was stirred at 0° C. for 0.5 h, and then 2-chloro-N-pyrimidin-2-yl-acetamide (Intermediate B3, 79.7 mg, 465 μmol) in DMF (0.5 mL) was added at 0° C. The RM was stirred at 25° C. for 2 h, quenched with water (1 mL) at 0° C. and extracted with EtOAc (3×1 mL). The combined organic layers were washed with brine (2×2 mL), dried over Na₂SO₄, filtered and concentrated under reduced pressure. Purification by prep-HPLC (column: Waters Xbridge BEH C18, 100×30 mm, 10 μm; mobile phase:water (10 mM NH₄HCO₃)−ACN]; B: 25-55%, 10 min) gave the title compound as a white solid. Y=22%. ¹H NMR (400 MHz, DMSO-d₆) δ 10.95 (s, 1H), 8.67 (d, J=5 Hz, 2H), 8.43 (d, J=8 Hz, 1H), 7.80 (d, J=8 Hz, 1H), 7.20 (t, J=5 Hz, 1H), 5.15 (s, 2H), 2.98 (q, J=7 Hz, 2H), 2.41-2.37 (m, 1H), 1.24 (t, J=7 Hz, 3H), 1.19-1.13 (m, 4H). LCMS (ESI): m/z: [M+H]⁺=351.2.

### Compound 33. 2-(2-Cyclopropyl-8-ethyl-5-oxo-pyrido[2,3-d]pyridazin-6-yl)-N-(5-fluoropyrimidin-2-yl)acetamide

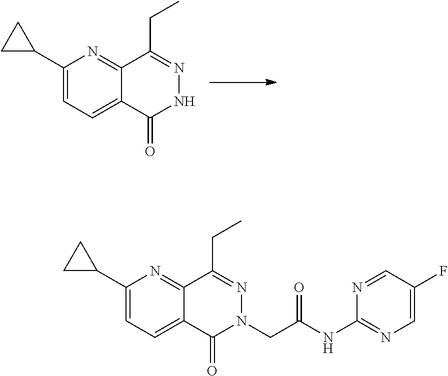 [SMILES: CCc1n[nH]c(=O)c2ccc(C3CC3)nc12 ; CCc1nn(CC(=O)Nc2ncc(F)cn2)c(=O)c2ccc(C3CC3)nc12]

Using 2-chloro-N-(5-fluoropyrimidin-2-yl)acetamide (Intermediate B2) and the same procedure as in Compound 32, the title compound was obtained as a white solid. Y=27%. ¹H NMR (400 MHz, DMSO-d₆) δ 11.11 (s, 1H), 8.77 (s, 2H), 8.43 (d, J=8 Hz, 1H), 7.80 (d, J=8 Hz, 1H), 5.10 (s, 2H), 2.98 (q, J=7 Hz, 2H), 2.41-2.38 (m, 1H), 1.24 (t, J=7 Hz, 3H), 1.20-1.10 (m, 4H). LCMS (ESI): m/z: [M+H]⁺=369.1.

### Compound 34. 2-(2-Cyclopropyl-8-methyl-5-oxo-pyrido[2,3-d]pyridazin-6-yl)-N-pyrimidin-2-yl-acetamide

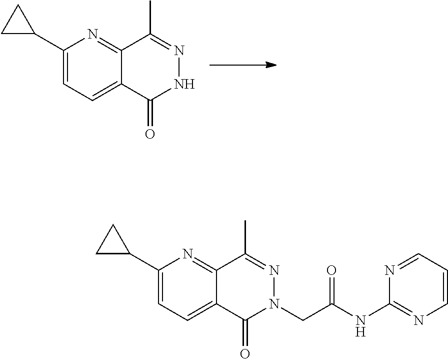 [SMILES: Cc1n[nH]c(=O)c2ccc(C3CC3)nc12 ; Cc1nn(CC(=O)Nc2ncccn2)c(=O)c2ccc(C3CC3)nc12]

Using 2-cyclopropyl-8-methyl-6H-pyrido[2,3-d]pyridazin-5-one (Intermediate C21), and 2-chloro-N-pyrimidin-2-yl-acetamide (Intermediate B3) and the same procedure as in Compound 32, title compound was obtained as a white solid. Y=19%. ¹H NMR (400 MHz, DMSO-d₆) δ 10.96 (s, 1H), 8.68 (d, J=5 Hz, 2H), 8.43 (d, J=8 Hz, 1H), 7.79 (d, J=8 Hz, 1H), 7.21 (t, J=5 Hz, 1H), 5.15 (s, 2H), 2.41 (s, 3H), 2.39-2.36 (m, 1H), 1.21-1.13 (m, 4H). ¹H NMR (400 MHz, MeOH-d₄) δ 8.63 (d, J=5 Hz, 2H), 8.47 (d, J=8 Hz, 1H), 7.72 (d, J=8 Hz, 1H), 7.17 (t, J=5 Hz, 1H), 5.26 (s, 2H), 2.60 (s, 3H), 2.40-2.30 (m, 1H), 1.29-1.16 (m, 4H). LCMS (ESI): m/z: [M+H]⁺=337.1.

### Compound 35. 2-(2-Cyclopropyl-8-methyl-5-oxo-pyrido[2,3-d]pyridazin-6-yl)-N-(5-fluoropyrimidin-2yl)acetamide

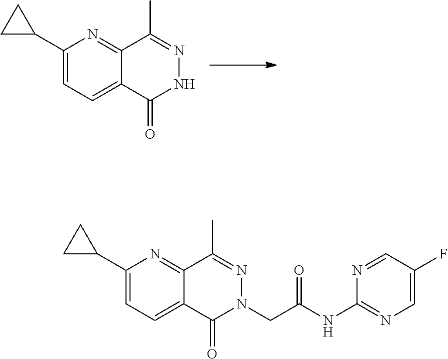 [SMILES: Cc1n[nH]c(=O)c2ccc(C3CC3)nc12 ; Cc1nn(CC(=O)Nc2ncc(F)cn2)c(=O)c2ccc(C3CC3)nc12]

Using 2-cyclopropyl-8-methyl-6H-pyrido[2,3-d]pyridazin-5-one (Intermediate C21) and 2-chloro-N-(5-fluoropyrimidin-2-yl)acetamide (Intermediate B3), and the same procedure as in Compound 32, the title compound was obtained as a white solid. Y=37%. ¹H NMR (400 MHz, DMSO-d₆) δ 11.10 (s, 1H), 8.77 (s, 2H), 8.43 (d, J=8 Hz, 1H), 7.79 (d, J=8 Hz, 1H), 5.09 (s, 2H), 2.52 (s, 3H), 2.41-2.37 (m, 1H), 1.23-1.13 (m, 4H). ¹H NMR (400 MHz, MeOH-d₄) δ 8.59 (s, 2H), 8.47 (d, J=8 Hz, 1H), 7.72 (d, J=8 Hz, 1H), 5.22 (s, 2H), 2.59 (s, 3H), 2.40-2.30 (m, 1H), 1.28-1.17 (m, 4H). LCMS (ESI): m/z: [M+H]⁺=355.2.

### Compound 40. 2-(2-Ethyl-7-(fluoromethyl)-4-oxo-2,4-dihydro-5H-pyrazolo[3,4-d]pyridazin-5-yl)-N-(pyrimidin-2-yl)acetamide

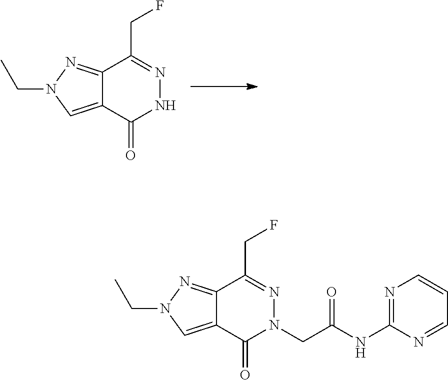 [SMILES: CCn1cc2c(=O)[nH]nc(CF)c2n1 ; CCn1cc2c(=O)n(CC(=O)Nc3ncccn3)nc(CF)c2n1]

To a solution of 2-ethyl-7-(fluoromethyl)-5H-pyrazolo[3,4-d]pyridazin-4-one (25 mg, 127 mol) in DMF (0.5 mL) at 0° C. was added NaH (15 mg, 382 mol, 60% in mineral oil) and the mixture was stirred for 30 mins, then a solution of 2-chloro-N-(pyrimidin-2-yl)acetamide (Intermediate B3, 33 mg, 191 mol) in DMF (0.5 mL) was added at 0° C. The reaction mixture was then stirred at 25° C. for 1 hr. The reaction mixture was quenched by addition of H₂O (0.5 mL) at 0° C. and then was extracted with EtOAc (3×0.5 mL). The combined organic layers were washed with brine (0.5 mL), dried over anhydrous Na₂SO₄, filtered and concentrated under reduced pressure. The crude product was purified by prep-HPLC (column: Waters Xbridge BEH C18 100×30 mm, 10 m; mobile phase: [(A) water (NH₄HCO₃)−(B) ACN]; B: 1-35%, 10 min) and lyophilised to give the title compound as a white solid, Y=19%. ¹H NMR (400 MHz, DMSO-d₆) δ 10.94 (s, 1H), 8.88 (s, 1H), 8.68 (d, J=5 Hz, 2H), 7.21 (t, J=5 Hz, 1H), 5.60 (s, 1H), 5.48 (s, 1H), 5.16 (s, 2H), 4.47 (q, J=7 Hz, 2H), 1.51 (t, J=7 Hz, 3H). LCMS (ESI): m/z [M+H]⁺=332.2.

### Compound 50. 1-(2,7-Diisopropyl-4-oxo-2,4-dihydro-5H-pyrazolo[3,4-d]pyridazin-5-yl)-N-(pyrimidin-2-yl)cyclopropane-1-carboxamide

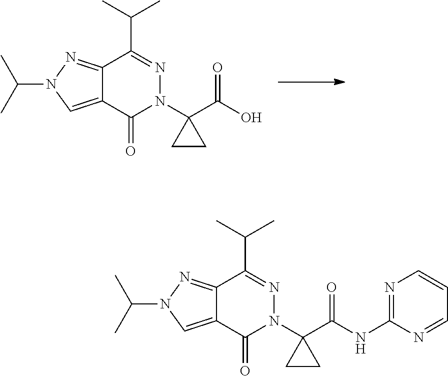 [SMILES: CC(C)c1nn(C2(C(=O)O)CC2)c(=O)c2cn(C(C)C)nc12 ; CC(C)c1nn(C2(C(=O)Nc3ncccn3)CC2)c(=O)c2cn(C(C)C)nc12]

To a stirred solution of pyrimidin-2-amine (38 mg, 394 μmol) in ACN (1 mL) at 25° C. was added 1-(2,7-diisopropyl-4-oxo-pyrazolo[3,4-d]pyridazin-5-yl)cyclopropanecarboxylic acid (Intermediate C23, 80 mg, 263 mol), NMM (29 μL, 263 μmol) and COMU (146 mg, 342 mol). The mixture was then stirred at 80° C. for 8 h. The reaction mixture was concentrated under reduced pressure. The crude material was purified by prep-HPLC (column: Waters Xbridge BEH C18 100×30 mm, 10 m; mobile phase: [(A) water (NH₄HCO₃)−(B) ACN]; B: 25-50%, 10 min) and lyophilised to give the title compound as a white solid, Y=35%. ¹H NMR (400 MHz, DMSO-d₆) δ 10.04 (s, 1H), 8.78 (s, 1H), 8.65 (d, J=5 Hz, 2H), 7.21 (t, J=5 Hz, 1H), 4.86-4.77 (m, 1H), 3.28-3.26 (m, 1H), 1.78-1.70 (m, 2H), 1.52 (d, J=7 Hz, 6H), 1.40-1.37 (m, 2H), 1.35 (d, J=7 Hz, 6H). LC-MS (ESI): m/z [M+H]⁺=382.2.

### Compound 51. 1-(2,7-Diisopropyl-4-oxo-2,4-dihydro-5H-pyrazolo[3,4-d]pyridazin-5-yl)-N-(5-fluoropyrimidin-2-yl)cyclopropane-1-carboxamide

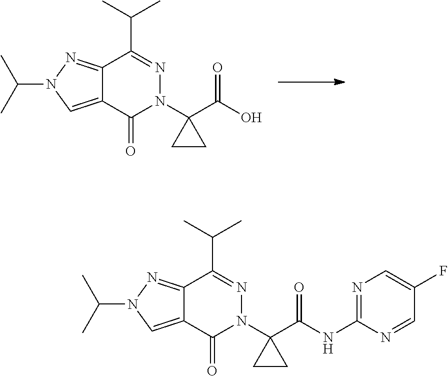 [SMILES: CC(C)c1nn(C2(C(=O)O)CC2)c(=O)c2cn(C(C)C)nc12 ; CC(C)c1nn(C2(C(=O)Nc3ncc(F)cn3)CC2)c(=O)c2cn(C(C)C)nc12]

To a solution of 1-(2,7-diisopropyl-4-oxo-pyrazolo[3,4-d]pyridazin-5-yl)cyclopropane carboxylic acid (30 mg, 99 mol) in ACN (0.5 mL) at 25° C. was added 5-fluoropyrimidin-2-amine (17 mg, 148 mol), COMU (55 mg, 128 mmol) and NMM (99 mol, 11 μL). The mixture was stirred at 80° C. for 8 h. The reaction mixture was concentrated under reduced pressure. The crude material was purified by prep-HPLC (column: Waters Xbridge Prep OBD C18 150×40 mm, 10 m; mobile phase: [(A) water (NH₄HCO₃)−(B) ACN]; B: 25-60%, 8 min) and lyophilised to give the title compound as a white solid, Y=63%. ¹H NMR (400 MHz, DMSO-d₆) δ 10.25 (s, 1H), 8.78-8.73 (m, 3H), 4.87-4.76 (m, 1H), 3.29-3.22 (m, 1H), 1.77-1.69 (m, 2H), 1.52 (d, J=7 Hz, 6H), 1.39-1.36 (m, 2H), 1.34 (d, J=7 Hz, 6H). LC-MS (ESI): m/z [M+H]⁺400.2.

### Compound 52. 2-(2-Ethyl-4-isopropyl-7-oxofuro[2,3-d]pyridazin-6(7H)-yl)-N-(pyrimidin-2-yl)acetamide

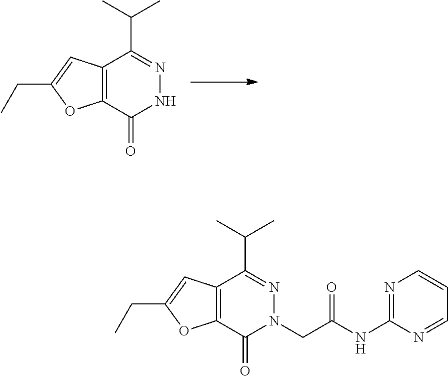 [SMILES: CCc1cc2c(C(C)C)n[nH]c(=O)c2o1 ; CCc1cc2c(C(C)C)nn(CC(=O)Nc3ncccn3)c(=O)c2o1]

To a solution of 2-ethyl-4-isopropyl-6H-furo[2,3-d]pyridazin-7-one (Intermediate C25, 70 mg, 339 μmol) in DMF (2 mL) was added NaH (54 mg, 1.36 mmol, 60% in mineral oil) at 0° C., the mixture was stirred at 0° C. for 0.5 h, and then was added dropwise a solution of 2-chloro-N-pyrimidin-2-yl-acetamide (87 mg, 509 mol) in DMF (1 mL) at 0° C. The mixture was stirred at 25° C. for 5.5 h under N₂. The mixture was quenched by addition of saturated aqueous NH₄Cl (5 mL) and the resulting mixture was extracted with EtOAc (3×5 mL). The combined organic layers were washed with brine (2 mL), dried over Na₂SO₄, filtered and the filtrate was concentrated under reduced pressure. The residue was purified by prep-HPLC (column: Phenomenex C18 75×30 mm, 3 m; mobile phase: [(A) water (NH₄HCO₃)−(B) ACN]; B: 20-30%, 8 min) and lyophilised to give the title compound as a white solid, Y=17%. ¹H NMR (400 MHz, DMSO-d₆) δ 10.93 (s, 1H), 8.68 (d, J=5 Hz, 2H), 7.20 (t, J=5 Hz, 1H), 6.90 (s, 1H), 5.18 (s, 2H), 3.19-3.13 (m, 1H), 2.86 (q, J=8 Hz, 2H), 1.36-1.20 (m, 9H). LCMS (ESI): m/z [M+H]⁺=342.2.

### Compound 53. 2-(2-Ethyl-4-isopropyl-7-oxofuro[2,3-d]pyridazin-6(7H)-yl)-N-(5-fluoropyrimidin-2-yl)acetamide

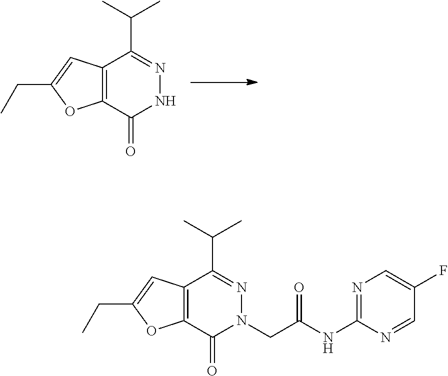 [SMILES: CCc1cc2c(C(C)C)n[nH]c(=O)c2o1 ; CCc1cc2c(C(C)C)nn(CC(=O)Nc3ncc(F)cn3)c(=O)c2o1]

The reaction was performed in the same manner as compound 52, using 2-ethyl-4-isopropyl-6H-furo[2,3-d]pyridazin-7-one (Intermediate C25, 60 mg, 291 mol) and 2-chloro-N-(5-fluoropyrimidin-2-yl)acetamide (Intermediate B2, 83 mg, 436 mol). The crude material was purified by prep-HPLC (column: Waters Xbridge BEH C18 100×30 mm, 10 m; mobile phase: [(A) water (NH₄HCO₃)−(B) ACN]B: 25-55%, 10 min) and lyophilised to give the title compound as a white solid, Y=19%. ¹H NMR (400 MHz, DMSO-d₆) δ 11.09 (s, 1H), 8.77 (s, 2H), 6.90 (s, 1H), 5.12 (s, 2H), 3.22-3.10 (m, 1H), 2.86 (q, J=8 Hz, 2H), 1.32-1.23 (m, 9H). LCMS (ESI): m/z [M+H]⁺=360.2.

### Compound 54. 2-(2,7-Diethyl-4-oxofuro[2,3-d]pyridazin-5(4H)-yl)-N-(pyrimidin-2-yl)acetamide

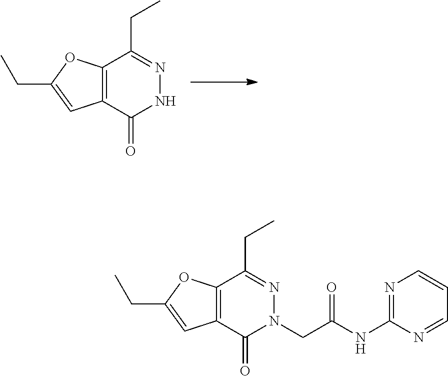 [SMILES: CCc1cc2c(=O)[nH]nc(CC)c2o1 ; CCc1cc2c(=O)n(CC(=O)Nc3ncccn3)nc(CC)c2o1]

The reaction was performed in the same manner as compound 52, using 2,7-diethylfuro[2,3-d]pyridazin-4(5H)-one (Intermediate C26, 60 mg, 312 mol) and 2-chloro-N-pyrimidin-2-yl-acetamide (Intermediate B3, 80 mg, 468 mol). The crude material was purified by prep-HPLC (column: Waters Xbridge BEH C18 100×30 mm, 10 m; mobile phase: [(A) water (NH₄HCO₃)−(B) ACN]B: 20-50%, 10 min) and lyophilised to the title compound as a white solid, Y=35%. ¹H NMR (400 MHz, DMSO-d₆) δ 10.93 (s, 1H), 8.67 (d, J=5 Hz, 2H), 7.20 (t, J=5 Hz, 1H), 6.82 (s, 1H), 5.17 (s, 2H), 2.90-2.80 (m, 4H), 1.30-1.20 (m, 6H). LCMS (ESI): m/z [M+H]⁺=328.2.

### Compound 55. 2-(2,7-Diethyl-4-oxofuro[2,3-d]pyridazin-5(4H)-yl)-N-(5-fluoropyrimidin-2-yl)acetamide

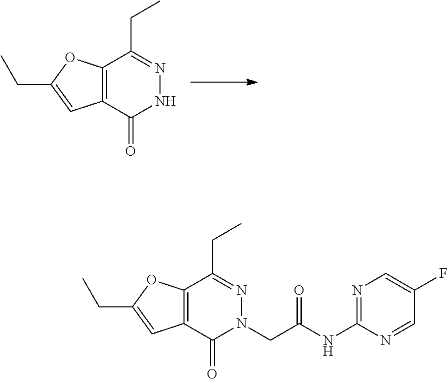 [SMILES: CCc1cc2c(=O)[nH]nc(CC)c2o1 ; CCc1cc2c(=O)n(CC(=O)Nc3ncc(F)cn3)nc(CC)c2o1]

The reaction and work-up was performed in the same manner as compound 52, using 2,7-diethyl-5H-furo[2,3-d]pyridazin-4-one (Intermediate C26, 60 mg, 312 mol) and 2-chloro-N-(5-fluoropyrimidin-2-yl)acetamide (Intermediate B2, 89 mg, 468 mol). The crude material was purified by prep-HPLC (column: Waters Xbridge BEH C₁₈ 100×30 mm, 10 m; mobile phase: [(A) water (NH₄HCO₃)−(B) ACN]B: 25-55%, 10 min) and lyophilised to give the title compound as a white solid, Y=45%. ¹H NMR (400 MHz, DMSO-d₆) δ 11.08 (s, 1H), 8.77 (s, 2H), 6.82 (s, 1H), 5.11 (s, 2H), 2.89-2.81 (m, 4H), 1.35-1.20 (m, 6H). LCMS (ESI): m/z [M+H]⁺=346.2.

### Compound 56. 2-(2-Ethyl-7-methyl-4-oxofuro[2,3-d]pyridazin-5(4H)-yl)-N-(pyrimidin-2-yl)acetamide

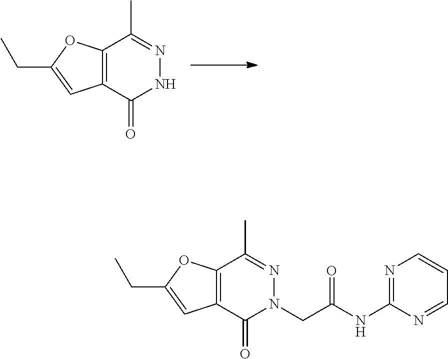 [SMILES: CCc1cc2c(=O)[nH]nc(C)c2o1 ; CCc1cc2c(=O)n(CC(=O)Nc3ncccn3)nc(C)c2o1]

The reaction and work-up was performed in the same manner as compound 52, using 2-ethyl-7-methyl-5H-furo[2,3-d]pyridazin-4-one (Intermediate C27, 30 mg, 168 mol) and 2-chloro-N-pyrimidin-2-yl-acetamide (Intermediate B3, 58 mg, 337 mol). The residue was purified by prep-HPLC (column: Waters Xbridge BEH C₁₈ 100×30 mm, 10 m; mobile phase: [(A) water (NH₄HCO₃)−(B) ACN]B: 15-45%, 10 min) and lyophilised to the title compound as a white solid, Y=19%. ¹H NMR (400 MHz, DMSO-d₆) δ 10.92 (s, 1H), 8.68 (d, J=5 Hz, 2H), 7.20 (t, J=5 Hz, 1H), 6.83 (s, 1H), 5.16 (s, 2H), 2.89 (q, J=8 Hz, 2H), 2.45 (s, 3H), 1.28 (t, J=8 Hz, 3H). LCMS (ESI): m/z [M+H]⁺=314.1.

### Compound 57. 2-(2-Ethyl-7-methyl-4-oxofuro[2,3-d]pyridazin-5(4H)-yl)-N-(5-fluoropyrimidin-2-yl)acetamide

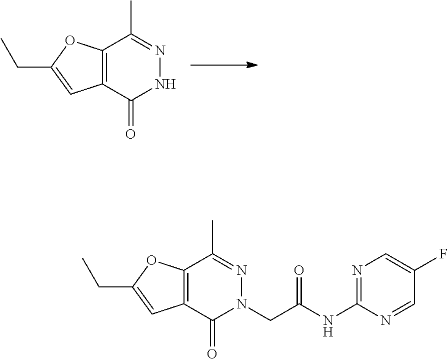 [SMILES: CCc1cc2c(=O)[nH]nc(C)c2o1 ; CCc1cc2c(=O)n(CC(=O)Nc3ncc(F)cn3)nc(C)c2o1]

The reaction and work-up was performed in the same manner as Compound 52, using 2-ethyl-7-methyl-5H-furo[2,3-d]pyridazin-4-one (Intermediate C27, 10 mg, 56 mol) and 2-chloro-N-(5-fluoropyrimidin-2-yl)acetamide (21 mg, 112 mol). The residue was combined another batch of residue from the same scale reaction, and the combined residue was purified by prep-HPLC (column: Waters Xbridge BEH C₁₈ 100×30 mm, 10 m; mobile phase: [(A) water (NH₄HCO₃)−(B) ACN]; B: 20-50%, 10 min) and lyophilised to give the title compound as a white solid, Y=26%. ¹H NMR (400 MHz, DMSO-d₆) δ 11.08 (s, 1H), 8.77 (s, 2H), 6.82 (s, 1H), 5.10 (s, 2H), 2.84 (q, J=8 Hz, 2H), 2.45 (s, 3H), 1.28 (t, J=8 Hz, 3H). LCMS (ESI): m/z [M+H]⁺=332.1.

### Compound 58. 2-(2-Chloro-7-isopropyl-4-oxofuro[2,3-d]pyridazin-5(4H)-yl)-N-(5-fluoropyrimidin-2-yl)acetamide

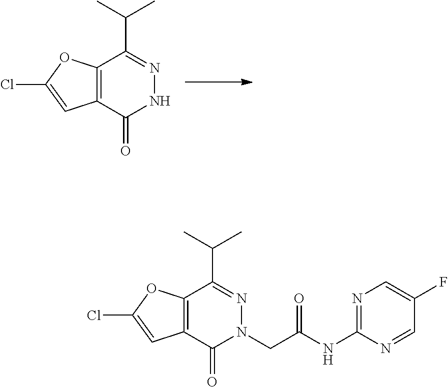 [SMILES: CC(C)c1n[nH]c(=O)c2cc(Cl)oc12 ; CC(C)c1nn(CC(=O)Nc2ncc(F)cn2)c(=O)c2cc(Cl)oc12]

To a solution of 2-chloro-7-isopropyl-5H-furo[2,3-d]pyridazin-4-one (Intermediate C30, 50 mg, 235 mol) in DMF (1 mL) was added Cs₂CO₃ (213 mg, 653 mol) at 25° C. and the mixture was stirred at 25° C. for 1 h. Then 2-chloro-N-(5-fluoropyrimidin-2-yl)acetamide (Intermediate B2 103 mg, 544 mol) was added at 25° C. and the mixture was stirred 2 h under N₂. The reaction mixture was diluted with H₂O (0.5 mL) at 25° C. The reaction mixture was directly purified by prep-HPLC (column: Waters Xbridge Prep OBD C18 150×40 mm, 10 m; mobile phase: [(A) water (NH₄HCO₃)−(B) ACN]; B: 25-55%, 8 min) and lyophilised to give the title compound as a white solid, Y=16%. ¹H NMR (400 MHz, DMSO-d₆) δ 11.11 (s, 1H), 8.77 (s, 2H), 7.32 (s, 1H), 5.12 (s, 2H), 3.30-3.20 (m, 1H), 1.29 (d, J=7 Hz, 6H). LCMS (ESI): m/z [M+H]⁺=366.1.

### Compound 59. 2-(2-Chloro-7-isopropyl-4-oxothieno[2,3-d]pyridazin-5(4H)-yl)-N-(5-fluoropyrimidin-2-yl)acetamide

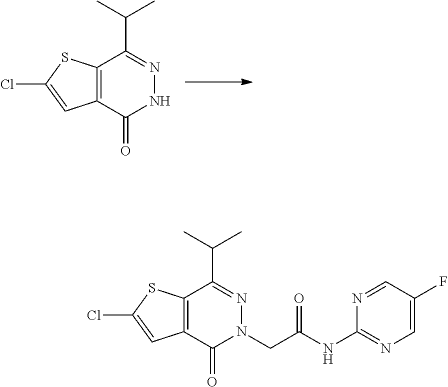 [SMILES: CC(C)c1n[nH]c(=O)c2cc(Cl)sc12 ; CC(C)c1nn(CC(=O)Nc2ncc(F)cn2)c(=O)c2cc(Cl)sc12]

The reaction was performed in the same manner as Compound 52, using 2-chloro-7-isopropyl-5H-thieno[2,3-d]pyridazin-4-one (Intermediate C31, 45 mg, 197 mol) and 2-chloro-N-(5-fluoropyrimidin-2-yl)acetamide (Intermediate B2, 75 mg, 394 mol. The crude material was purified by prep-HPLC (column: Waters Xbridge Prep OBD C18 150×40 mm, m; mobile phase: [(A) water (NH₄HCO₃)−(B) ACN]; B: 28-58%, 8 min) and lyophilised to give the title compound as a white solid, Y=23%. ¹H NMR (400 MHz, DMSO-d₆) δ 11.12 (s, 1H), 8.77 (s, 2H), 7.76 (s, 1H), 5.11 (s, 2H), 3.15-3.05 (m, 1H), 1.29 (d, J=7 Hz, 6H). LCMS (ESI): m/z: [M+H]⁺=382.1.

### Compound 60. 2-(2-Chloro-4-isopropyl-7-oxothieno[2,3-d]pyridazin-6(7H)-yl)-N-(5-fluoropyrimidin-2-yl)acetamide

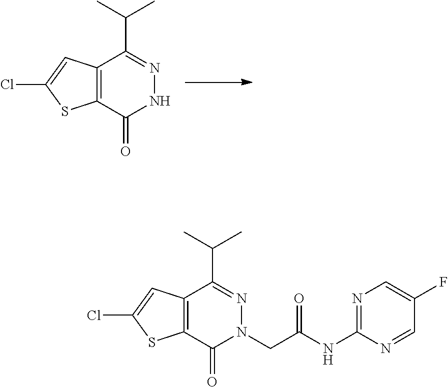 [SMILES: CC(C)c1n[nH]c(=O)c2sc(Cl)cc12 ; CC(C)c1nn(CC(=O)Nc2ncc(F)cn2)c(=O)c2sc(Cl)cc12]

The reaction was performed in the same manner as Compound 52, using 2-chloro-4-isopropyl-6H-thieno[2,3-d]pyridazin-7-one (Intermediate C9, 70 mg, 306 mol) and 2-chloro-N-(5-fluoropyrimidin-2-yl)acetamide (Intermediate B2, 87 mg, 459 mol). The crude material was purified by prep-HPLC (column: Waters Xbridge BEH C18 100×30 mm, 10 m; mobile phase: [(A) water (NH₄HCO₃)−(B) ACN]; B: 30-60%, 10 min) and lyophilised to give the title compound as a white solid, Y=33%. 1H NMR (400 MHz, DMSO-d₆) δ 11.13 (s, 1H), 8.77 (s, 2H), 7.91 (s, 1H), 5.13 (s, 2H), 3.32-3.25 (m, 1H), 1.25 (d, J=7 Hz, 6H). LCMS (ESI): m/z [M+H]⁺=382.1.

### Compound 62. 2-(2-Bromo-7-isopropyl-4-oxofuro[2,3-d]pyridazin-5(4H)-yl)-N-(pyrimidin-2-yl)acetamide

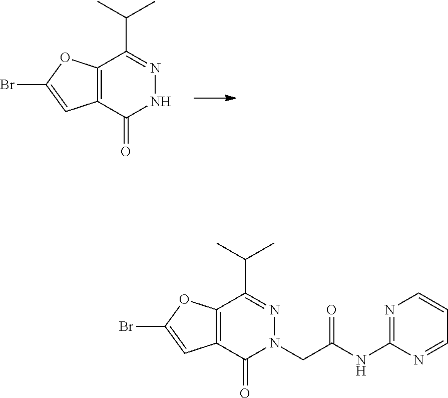 [SMILES: CC(C)c1n[nH]c(=O)c2cc(Br)oc12 ; CC(C)c1nn(CC(=O)Nc2ncccn2)c(=O)c2cc(Br)oc12]

The reaction was performed in the same manner as Compound 52, using 2-bromo-7-isopropyl-5H-furo[2,3-d]pyridazin-4-one (Intermediate C32, 50 mg, 194 mol) and 2-chloro-N-pyrimidin-2-yl-acetamide (Intermediate B3, 67 mg, 389 mol). The reaction mixture was quenched by addition of H₂O (0.5 mL), and the resulting mixture was directly purified by prep-HPLC (column: Waters Xbridge BEH C₁₈ 100×30 mm, 10 m; mobile phase: [(A) water (NH₄HCO₃)−(B) ACN]; B: 25-55%, 10 min) and lyophilised to give the title compound as a white solid, Y=13%. ¹H NMR (DMSO-d₆) δ 10.95 (s, 1H), 8.67 (d, J=5 Hz, 2H), 7.39 (s, 1H), 7.21 (t, J=5 Hz, 1H), 5.18 (s, 2H), 3.22-3.27 (m, 1H), 1.30 (d, J=7 Hz, 6H). LCMS (ESI): m/z [M+H]⁺=392.1.

### Compound 63. 2-(2,7-Diisopropyl-4-oxo-pyrazolo[3,4-d]pyridazin-5-yl)-N-(5-methylpyrimidin-2-yl)acetamide

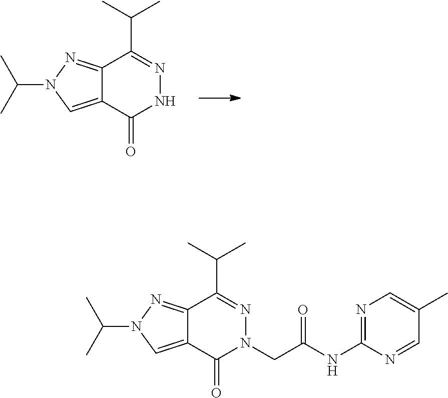 [SMILES: CC(C)c1n[nH]c(=O)c2cn(C(C)C)nc12 ; Cc1cnc(NC(=O)Cn2nc(C(C)C)c3nn(C(C)C)cc3c2=O)nc1]

To a stirred solution of 2,7-diisopropyl-2,5-dihydro-4H-pyrazolo[3,4-d]pyridazin-4-one (Intermediate C12, 70 mg, 318 mol) in DMF (0.5 mL) at 0° C. was added a solution LiHMDS (1 M in THF, 794 μL, 794 μmol) and the resulting mixture stirred at 0° C. for 30 min. Then to above mixture was added a solution of 2-chloro-N-(5-methylpyrimidin-2-yl)acetamide (Intermediate B5, 88 mg, 477 mol) in DMF (0.5 mL) at 0° C., the resulting mixture was stirred at 25° C. for 2 h. The reaction mixture was quenched by addition of saturated NH₄Cl aqueous solution (2 mL), and extracted with EtOAc (3×2 mL). The combined organic layers were washed with brine (2 mL), dried over Na₂SO₄, filtered and concentrated under reduced pressure. The crude material was purified by prep-HPLC (column: Waters Xbridge Prep OBD C₁₈ 150×40 mm, 10 m; mobile phase: [(A) water (NH₄HCO₃)−(B) ACN]; B: 20-50%, 8 min) and lyophilised to give the title compound as a white solid, Y=15%. 1H NMR (400 MHz, DMSO-d₆) δ 10.75 (s, 1H), 8.80 (s, 1H), 8.52 (s, 2H), 5.04 (s, 2H), 4.87-4.78 (m, 1H), 3.27-3.25 (m, 1H), 2.22 (s, 3H), 1.54 (d, J=7 Hz, 6H), 1.32 (d, J=7 Hz, 6H). LC-MS (ESI): m/z [M+H]⁺=370.2.

### Compound 64. 2-(2-Bromo-4-isopropyl-7-oxothieno[2,3-d]pyridazin-6(7H)-yl)-N-(pyrimidin-2-yl)acetamide

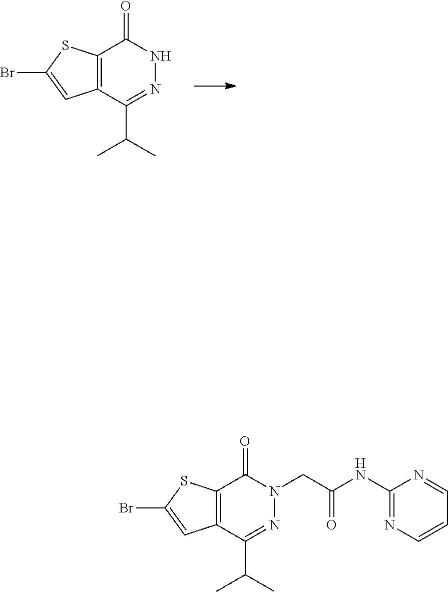 [SMILES: CC(C)c1n[nH]c(=O)c2sc(Br)cc12 ; CC(C)c1nn(CC(=O)Nc2ncccn2)c(=O)c2sc(Br)cc12]

The reaction was performed in the same manner as compound 52, using 2-bromo-4-isopropyl-6H-thieno[2,3-d]pyridazin-7-one (Intermediate C34, 140 mg, 513 mol) and 2-chloro-N-pyrimidin-2-yl-acetamide (Intermediate B3, 264 mg, 1.54 mmol). The crude product was purified by prep-HPLC (column: Waters Xbridge Prep OBD C18 150×40 mm, 10 m; mobile phase: [(A) water (NH₄HCO₃)−(B) ACN]; B: 15-45%, 8 min) and lyophilised to give the title compound as a white solid, Y=24%. ¹H NMR (400 MHz, DMSO-d₆) δ 10.97 (s, 1H), 8.68 (d, J=5 Hz, 2H), 7.99 (s, 1H), 7.21 (t, J=5 Hz, 1H), 5.19 (s, 2H), 3.39-3.33 (m, 1H), 1.25 (d, J=7 Hz, 6H). LCMS (ESI): m/z [M+H]⁺=408.0.

### Compound 65. 2-(2-Cyclopropyl-7-isopropyl-4-oxo-2,4-dihydro-5H-pyrazolo[3,4-d]pyridazin-5-yl)-N-(pyrimidin-2-yl)acetamide

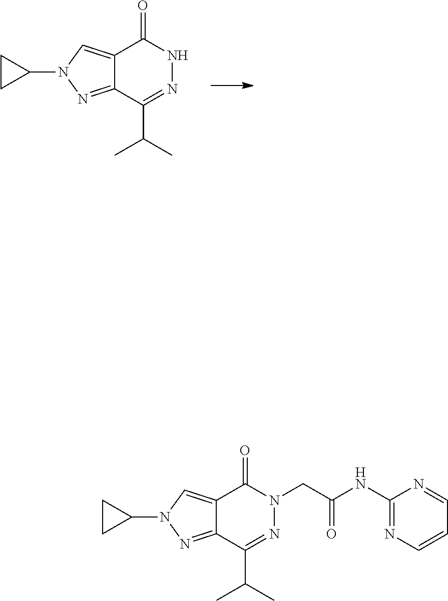 [SMILES: CC(C)c1n[nH]c(=O)c2cn(C3CC3)nc12 ; CC(C)c1nn(CC(=O)Nc2ncccn2)c(=O)c2cn(C3CC3)nc12]

The reaction was performed in the same manner as Compound 52, using 2-cyclopropyl-7-isopropyl-5H-pyrazolo[3,4-d]pyridazin-4-one (Intermediate C13, 100 mg, 458 mol) and 2-chloro-N-pyrimidin-2-yl-acetamide (Intermediate B3, 157 mg, 916 mol) at 0° C. The crude product was purified by prep-HPLC (column: Waters Xbridge Prep OBD C₁₈ 150×40 mm, 10 μm; mobile phase: [(A) water (NH₄HCO₃)−(B) ACN]; B: 15-45%, 8 min) to give the title compound as a white solid, Y=19%. ¹H NMR (400 MHz, DMSO-d₆) δ 10.91-10.61 (m, 1H), 8.82 (s, 1H), 8.67 (d, J=5 Hz, 2H), 7.20 (t, J=5 Hz, 1H), 5.07 (s, 2H), 4.14-4.06 (m, 1H), 3.29-3.17 (m, 1H), 1.30 (d, J=7 Hz, 6H), 1.29-1.25 (m, 2H), 1.17-1.06 (m, 2H). LCMS (ESI): m/z [M+H]⁺=354.0.

### Compound 66. 1-(2-Cyclopropyl-7-isopropyl-4-oxo-2,4-dihydro-5H-pyrazolo[3,4-d]pyridazin-5-yl)-N-(pyrimidin-2-yl)cyclopropane-1-carboxamide

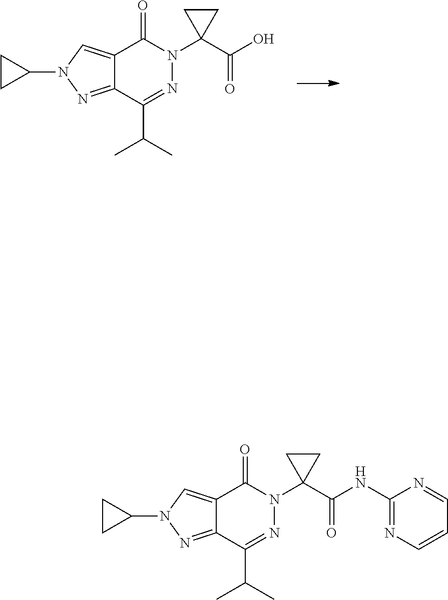 [SMILES: CC(C)c1nn(C2(C(=O)O)CC2)c(=O)c2cn(C3CC3)nc12 ; CC(C)c1nn(C2(C(=O)Nc3ncccn3)CC2)c(=O)c2cn(C3CC3)nc12]

The reaction was performed in the same manner as Compound 50, using 1-(2-cyclopropyl-7-isopropyl-4-oxo-pyrazolo[3,4-d]pyridazin-5-yl)cyclopropanecarboxylic acid (Intermediate C35, 100 mg, 331 mol) and pyrimidin-2-amine (47 mg, 496 mol). The crude material was purified by prep-HPLC (column: Waters Xbridge Prep OBD C18 150×40 mm, m; mobile phase: [(A) water (NH₄HCO₃)−(B) ACN]; B: 15-45%, 8 mins) and lyophilised to give the title compound as a white solid, Y=19%. ¹H NMR (400 MHz, DMSO-d₆) δ 10.00 (s, 1H), 8.72 (s, 1H), 8.65 (d, J=5 Hz, 2H), 7.21 (t, J=5 Hz, 1H), 4.20-4.02 (m, 1H), 3.35-3.15 (m, 1H), 1.86-1.68 (m, 2H), 1.41-1.31 (m, 8H), 1.26-1.21 (m, 2H), 1.17-1.08 (m, 2H). LCMS (ESI): m/z [M+H]⁺=380.2.

### Compound 67. N-(5-Chloropyrimidin-2-yl)-2-(2,7-diisopropyl-4-oxo-2,4-dihydro-5H-pyrazolo[3,4-d]pyridazin-5-yl)acetamide

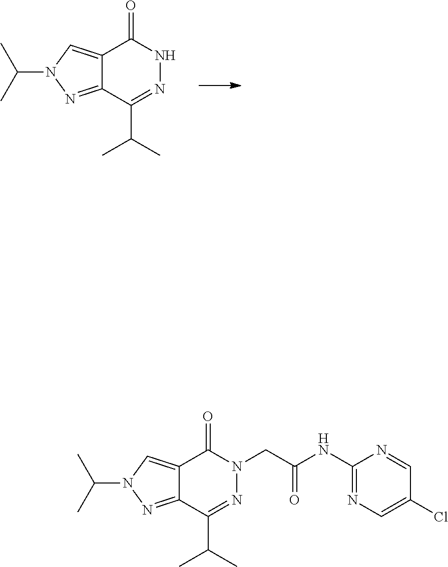 [SMILES: CC(C)c1n[nH]c(=O)c2cn(C(C)C)nc12 ; CC(C)c1nn(CC(=O)Nc2ncc(Cl)cn2)c(=O)c2cn(C(C)C)nc12]

To a stirred solution of 2,7-diisopropyl-2,5-dihydro-4H-pyrazolo[3,4-d]pyridazin-4-one (Intermediate C22, 100 mg, 454 mol) in DMF (3 mL) at 0° C. was added NaH (54 mg, 1.36 mmol, 60% in mineral oil). The mixture was stirred at 0° C. for 0.5 h, then 2-chloro-N-(5-chloropyrimidin-2-yl)acetamide (Intermediate B4, 187 mg, 908 mol) was added. The mixture was allowed to warm to 25° C. then was stirred for a further 2 h. The reaction mixture was quenched by addition of saturated NH₄Cl_{(aq)} solution (2 mL) at 0° C. The resulting mixture was extracted with ethyl acetate (3×2 mL) and the combined organic layers were washed with brine (2 mL), dried over anhydrous Na₂SO₄, filtered and concentrated under reduced pressure. The crude material was purified by prep-HPLC (column: Waters Xbridge BEH C18 100×30 mm, 10 m; mobile phase: [(A) NH₄HCO_{3(aq)} solution−(B) ACN]; B: 30-60%, 10 min) and lyophilised to the title compound as a white solid, Y=25%. ¹H NMR (400 MHz, DMSO-d₆) δ 11.09 (s, 1H), 8.81 (s, 1H), 8.78 (s, 2H), 5.04 (s, 2H), 4.88-4.77 (m, 1H), 3.31-3.25 (m, 1H), 1.54 (d, J=7 Hz, 6H), 1.32 (d, J=7 Hz, 6H). LCMS (ESI): m/z: [M+H]⁺=390.1.

### Compound 68. 2-(2,7-Diisopropyl-4-oxo-2,4-dihydro-5H-pyrazolo[3,4-d]pyridazin-5-yl)-N-((cis)-3-hydroxy-3-methylcyclobutyl)acetamide

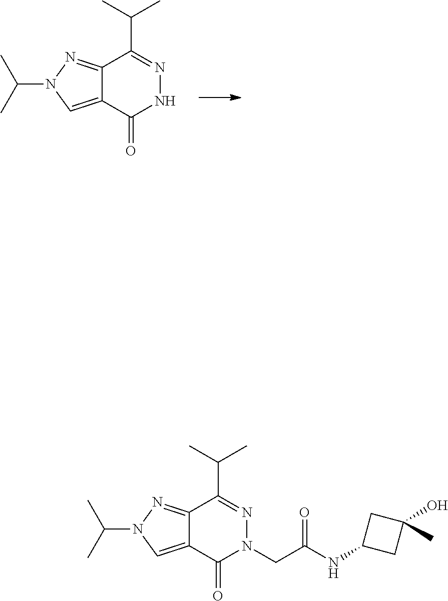 [SMILES: CC(C)c1n[nH]c(=O)c2cn(C(C)C)nc12 ; CC(C)c1nn(CC(=O)N[C@H]2C[C@@](C)(O)C2)c(=O)c2cn(C(C)C)nc12]

To a mixture of 2,7-diisopropyl-5H-pyrazolo[3,4-d]pyridazin-4-one (80 mg, 363 umol) in DMF (0.8 mL) was added cis-2-chloro-N-(3-hydroxy-3-methyl-cyclobutyl)acetamide (Intermediate B6, 97 mg, 545 mol) and Cs₂CO₃ (355 mg, 1.09 mmol) at 25° C. The mixture was stirred at 80° C. for 2 h. The reaction mixture was diluted with H₂O (2 mL) and extracted with EtOAc (3×2 mL). The combined organic layers were washed with brine (2 mL), dried over anhydrous Na₂SO₄, filtered and concentrated under reduced pressure. The crude material was purified by prep-HPLC (column: Waters Xbridge Prep OBD C18 150×40 mm, 10 m; mobile phase: [(A) water (NH₄HCO₃)−(B) ACN]; B: 25-55%, 8 min) and lyophilized to give the title compound as a white solid, Y=51%. ¹H NMR (400 MHz, DMSO-d₆) δ 8.77 (s, 1H), 8.16 (d, J=7 Hz, 1H), 4.95 (s, 1H), 4.85-4.75 (m, 1H), 4.56 (s, 2H), 3.83-3.71 (m, 1H), 3.30-3.22 (m, 1H), 2.25-2.15 (m, 2H), 2.01-1.86 (m, 2H), 1.53 (d, J=7 Hz, 6H), 1.31 (d, J=7 Hz, 6H), 1.21 (s, 3H). LCMS (ESI): m/z [M+H]⁺=362.1.

### Compound 69. 2-(2-Ethyl-4-isopropyl-7-oxothieno[2,3-d]pyridazin-6(7H)-yl)-N-((cis)-3-hydroxy-3-methylcyclobutyl)acetamide

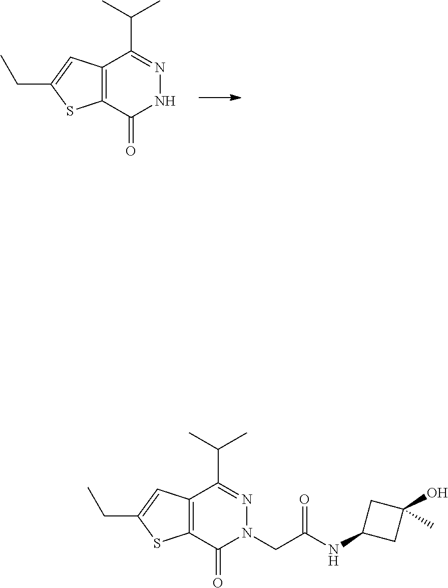 [SMILES: CCc1cc2c(C(C)C)n[nH]c(=O)c2s1 ; CCc1cc2c(C(C)C)nn(CC(=O)N[C@H]3C[C@@](C)(O)C3)c(=O)c2s1]

To a solution of 2-ethyl-4-isopropyl-6H-thieno[2,3-d]pyridazin-7-one (Intermediate C17, 100 mg, 450 mol) in DMF (1 mL) was added Cs₂CO₃ (440 mg, 1.35 mmol) and cis-2-chloro-N-(3-hydroxy-3-methyl-cyclobutyl) acetamide (Intermediate B6, 160 mg, 900 mol) at 25° C. The mixture was stirred at 80° C. for 2 h. The reaction mixture was diluted with H₂O (2 mL) and the resulting mixture extracted with EtOAc (2×2 mL). The combined organic layers were washed with brine (2 mL), dried over anhydrous Na₂SO₄, filtered and concentrated under reduced pressure. The crude material was purified by prep-HPLC (column: Phenomenex C18 80×40 mm, 3 m; mobile phase: [(A) water (NH₄HCO₃)−(B) ACN]; B: 10-40%, 8 min) to give the title compound, as a white solid, Y=32%. ¹H NMR (400 MHz, DMSO-d₆) δ 8.26 (d, J=7 Hz, 1H), 7.44 (s, 1H), 4.97 (s, 1H), 4.66 (s, 2H), 3.80-3.70 (m, 1H), 3.31-3.25 (m, 1H), 3.00 (q, J=8 Hz, 2H), 2.25-2.19 (m, 2H), 1.97-1.90 (m, 2H), 1.33 (t, J=8 Hz, 3H), 1.26-1.20 (m, 9H). LCMS (ESI): m/z [M+H]⁺=364.2.

### Compound 70. 1-(2-Cyclopropyl-7-isopropyl-4-oxo-2,4-dihydro-5H-pyrazolo[3,4-d]pyridazin-5-yl)-N-(pyrimidin-2-yl)cyclopropane-1-carboxamide

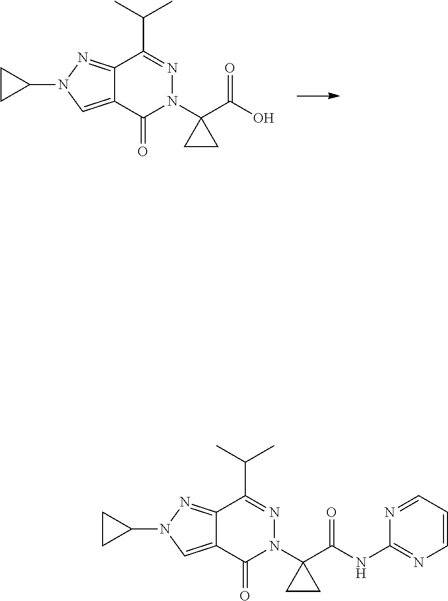 [SMILES: CC(C)c1nn(C2(C(=O)O)CC2)c(=O)c2cn(C3CC3)nc12 ; CC(C)c1nn(C2(C(=O)Nc3ncccn3)CC2)c(=O)c2cn(C3CC3)nc12]

To a solution of 1-(2-cyclopropyl-7-isopropyl-4-oxo-pyrazolo[3,4-d]pyridazin-5-yl)cyclo propanecarboxylic acid (Intermediate C35, 100 mg, 331 mol) in ACN (1 mL) at 25° C. was added pyrimidin-2-amine (47 mg, 496 mol), COMU (212 mg, 496 mol) and NMM (36 μL, 332 mol). The mixture was stirred at 80° C. for 2 h under N₂. The reaction mixture was diluted by addition of H₂O (1 mL) and the resulting mixture was extracted with EtOAc (3×1 mL). The combined organic layers were washed with brine (1 mL), dried over Na₂SO₄, filtered and concentrated under reduced pressure. The crude material was purified by prep-HPLC (column: Waters Xbridge Prep OBD C18 150×40 mm, 10 m; mobile phase: [(A) water (NH₄HCO₃)−(B) ACN]; B: 15-45%, 8 min) and lyophilised to give the title compound as a white solid, Y=9%. ¹H NMR (400 MHz, DMSO-d₆) δ 10.00 (s, 1H), 8.72 (s, 1H), 8.65 (d, J=5 Hz, 2H), 7.21 (t, J=5 Hz, 1H), 4.20-4.02 (m, 1H), 3.35-3.15 (m, 1H), 1.86-1.68 (m, 2H), 1.41-1.31 (m, 8H), 1.26-1.21 (m, 2H), 1.17-1.08 (m, 2H). LCMS (ESI): m/z [M+H]⁺=380.2.

### Compound 71. 2-(2-Cyclobutyl-7-isopropyl-4-oxo-2,4-dihydro-5H-pyrazolo[3,4-d]pyridazin-5-yl)-N-(pyrimidin-2-yl)acetamide

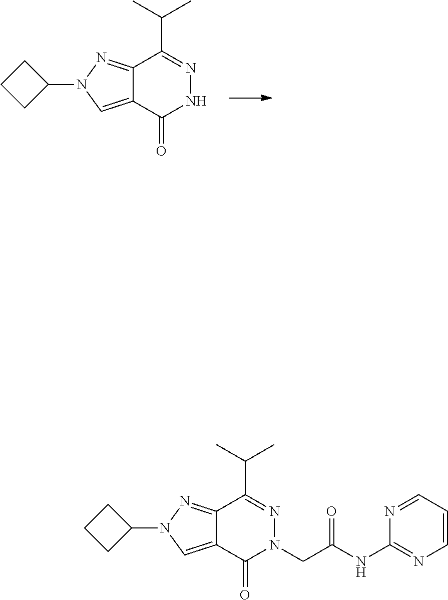 [SMILES: CC(C)c1n[nH]c(=O)c2cn(C3CCC3)nc12 ; CC(C)c1nn(CC(=O)Nc2ncccn2)c(=O)c2cn(C3CCC3)nc12]

To a solution of 2-cyclobutyl-7-isopropyl-5H-pyrazolo[3,4-d]pyridazin-4-one (Intermediate C36, 80 mg, 344 mol) in DMF (1 mL) at 0° C. was added LiHMDS (1 M in THF, 689 μL, 689 umol). The mixture was stirred at 0° C. for 30 min then a solution of 2-chloro-N-pyrimidin-2-yl-acetamide (Intermediate B3, 89 mg, 517 mol) in DMF (1 mL) added. The reaction mixture was stirred at 25° C. for 8 h then quenched by addition of saturated NH₄Cl_{(aq)} (2 mL) and extracted with EtOAc (3×2 mL). The combined organic layers were washed with brine (2 mL), dried over Na₂SO₄, filtered and concentrated under reduced pressure. The crude material purified by prep-HPLC (column: Phenomenex C18 75×30 mm, 3 μm; mobile phase: [(A) water (NH₄HCO₃)−(B) ACN]; B: 25-35%, 8 min) and lyophilised to give the title compound as a white solid, Y=26%. ¹H NMR (400 MHz, DMSO-d₆) δ 10.87 (s, 1H), 8.86 (s, 1H), 8.67 (d, J=5 Hz, 2H), 7.20 (t, J=5 Hz, 1H), 5.30-5.00 (m, 1H), 5.07 (s, 2H), 3.31-3.26 (m, 1H), 2.68-2.53 (m, 4H), 1.92-1.84 (m, 2H), 1.32 (d, J=7.0 Hz, 6H). LCMS (ESI): m/z [M+H]⁺=368.0.

### Compound 72. 2-(2,7-Diisopropyl-4-oxo-pyrazolo[3,4-d]pyridazin-5-yl)-N-oxazol-2-yl-acetamide

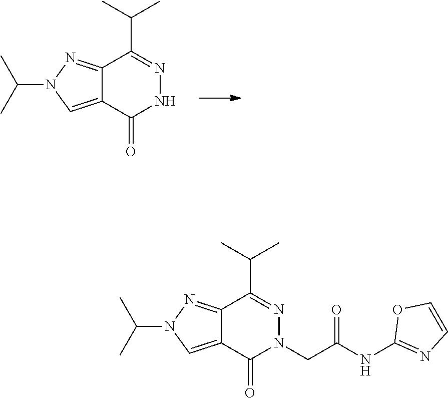 [SMILES: CC(C)c1n[nH]c(=O)c2cn(C(C)C)nc12 ; CC(C)c1nn(CC(=O)Nc2ncco2)c(=O)c2cn(C(C)C)nc12]

To a solution of 2,7-diisopropyl-5H-pyrazolo[3,4-d]pyridazin-4-one (Intermediate C12, 50 mg, 227 mol) in DMF (1 mL) at 25° C. was added t-BuOLi (55 mg, 681 μmol) and 2-chloro-N-oxazol-2-yl-acetamide (Intermediate B7, 55 mg, 340 mol), the mixture was stirred for 2 h. The reaction mixture was diluted with H₂O (1 mL) and extracted with EtOAc (3×1 mL). The combined organic layers were washed with brine (3×1 mL), dried over anhydrous Na₂SO₄, filtered and concentrated under reduced pressure to give a residue. The crude material was purified by prep-HPLC (column: Waters Xbridge BEH C18 100×30 mm, 10 m; mobile phase: [(A) water (NH₄HCO₃)−(B) ACN]; B: 20-50%, 8 min) and lyophilised to give the title compound as a white solid, Y=74%. ¹H NMR (400 MHz, DMSO-d₆) δ 11.41 (s, 1H), 8.81 (s, 1H), 7.87 (d, J=1 Hz, 1H), 7.11 (d, J=1 Hz, 1H), 4.90 (s, 2H), 4.86-4.77 (m, 1H), 3.31-3.27 (m, 1H), 1.54 (d, J=7 Hz, 6H), 1.32 (d, J=7 Hz, 6H). LCMS (ESI): m/z [M+H]+=345.2.

### Compound 73. 2-(2-Cyclopropyl-7-isopropyl-4-oxo-2,4-dihydro-5H-pyrazolo[3,4-d]pyridazin-5-yl)-N-(oxazol-2-yl)acetamide

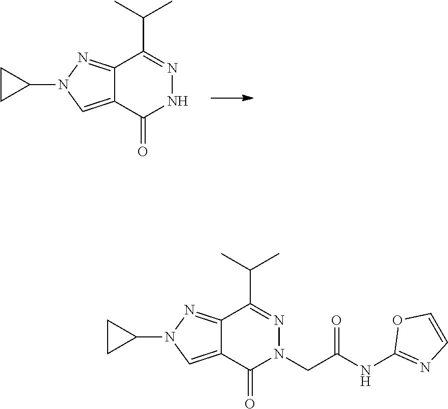 [SMILES: CC(C)c1n[nH]c(=O)c2cn(C3CC3)nc12 ; CC(C)c1nn(CC(=O)Nc2ncco2)c(=O)c2cn(C3CC3)nc12]

The reaction was performed in the same manner as Compound 72, using 2-cyclopropyl-7-isopropyl-5H-pyrazolo[3,4-d]pyridazin-4-one (Intermediate C13, 60 mg, 275 mol) and chloro-N-oxazol-2-yl-acetamide (66 mg, 412 mol). The crude material was purified by prep-HPLC (column: Waters Xbridge Prep OBD C₁₈ 150×40 mm, 10 m; mobile phase: [(A) water (NH₄HCO₃)−(B) ACN]; B: 15-45%, 8 min) and lyophilised to give the title compound as a white solid, Y=27%. ¹H NMR (400 MHz, DMSO-d₆) δ 11.50 (s, 1H), 8.84 (s, 1H), 7.88 (s, 1H), 7.11 (s, 1H), 7.10 (s, 2H), 4.20-4.10 (m, 1H), 3.30-3.20 (m, 1H), 1.30 (d, J=7 Hz, 6H), 1.24-1.29 (m, 2H), 1.09-1.16 (m, 2H). LCMS (ESI): m/z [M+H]⁺=343.2.

### Compound 74. 2-(2-Bromo-4-isopropyl-7-oxothieno[2,3-d]pyridazin-6(7H)-yl)-N-(cis-3-hydroxy-3-methylcyclobutyl)acetamide

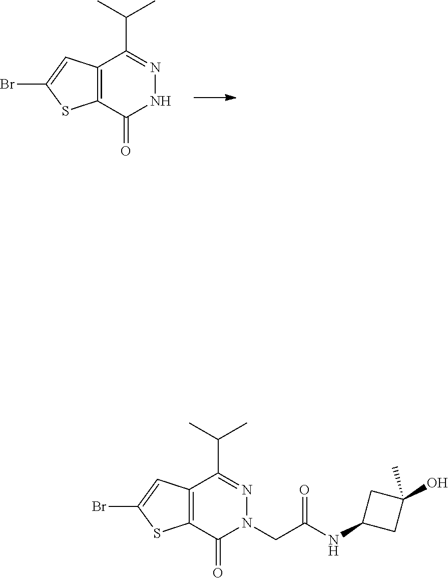 [SMILES: CC(C)c1n[nH]c(=O)c2sc(Br)cc12 ; CC(C)c1nn(CC(=O)N[C@H]2C[C@@](C)(O)C2)c(=O)c2sc(Br)cc12]

The reaction was performed in the same manner as compound 68, using 2-bromo-4-isopropyl-6H-thieno[2,3-d]pyridazin-7-one (Intermediate C34, 100 mg, 366 mol) and cis-2-chloro-N-(3-hydroxy-3-methyl-cyclobutyl) acetamide (Intermediate B6, 130 mg, 732 mol). The crude material was purified by prep-HPLC (column: Phenomenex C18 75×30 mm, 3 m; mobile phase: [(A) water (NH₄HCO₃)−(B) ACN]; B: 25-35%, 12 min) and lyophilised to give the title compound as a white solid, Y=35%. ¹H NMR (400 MHz, DMSO-d₆) δ 8.29 (d, J=7 Hz, 1H), 7.97 (s, 1H), 4.97 (s, 1H), 4.66 (s, 2H), 3.79-3.70 (m, 1H), 3.36-3.32 (m, 1H), 2.24-2.20 (m, 2H), 1.96-1.90 (m, 2H), 1.23 (d, J=7 Hz, 6H), 1.21 (s, 3H). LCMS (ESI): m/z [M+H]⁺=414.1.

### Compound 75. 2-(2-Cyclopropyl-4-isopropyl-7-oxothieno[2,3-d]pyridazin-6(7H)-yl)-N-(pyrimidin-2-yl)acetamide

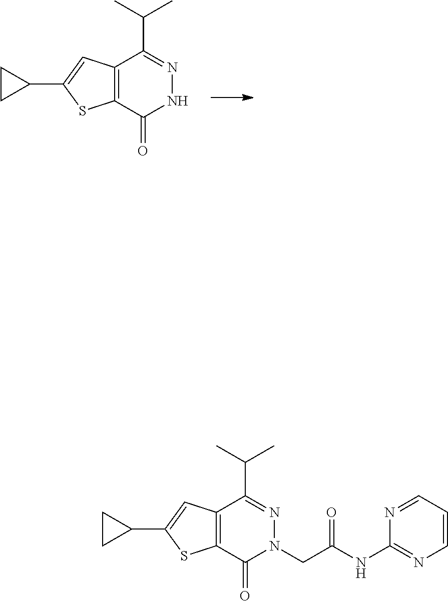 [SMILES: CC(C)c1n[nH]c(=O)c2sc(C3CC3)cc12 ; CC(C)c1nn(CC(=O)Nc2ncccn2)c(=O)c2sc(C3CC3)cc12]

The reaction was performed in the same manner as Compound 52, using 2-cyclopropyl-4-isopropyl-6H-thieno[2,3-d]pyridazin-7-one (Intermediate C37, 80 mg, 341 mol) 2-chloro-N-pyrimidin-2-yl-acetamide (Intermediate B3, 88 mg, 512 mol). The crude material was purified by prep-HPLC (column: Phenomenex C18 75×30 mm, 3 m; mobile phase: [(A) water (NH₄HCO₃)−(B) ACN]; B: 30-45%, 8 min) and lyophilised to give the title compound as a white solid, Y=9%. ¹H NMR (400 MHz, DMSO-d₆) δ 10.93 (s, 1H), 8.67 (d, J=5 Hz, 2H), 7.41 (s, 1H), 7.20 (t, J=5 Hz, 1H), 5.16 (s, 2H), 3.50-3.30 (m, 1H), 2.44-2.30 (m, 1H), 1.25 (d, J=7 Hz, 6H), 1.21-1.16 (m, 2H), 0.95-0.88 (m, 2H). LCMS (ESI): m/z [M+H]⁺=370.1.

### Compound 76. 2-(2-Cyclopropyl-4-isopropyl-7-oxothieno[2,3-d]pyridazin-6(7H)-yl)-N-(oxazol-2-yl)acetamide

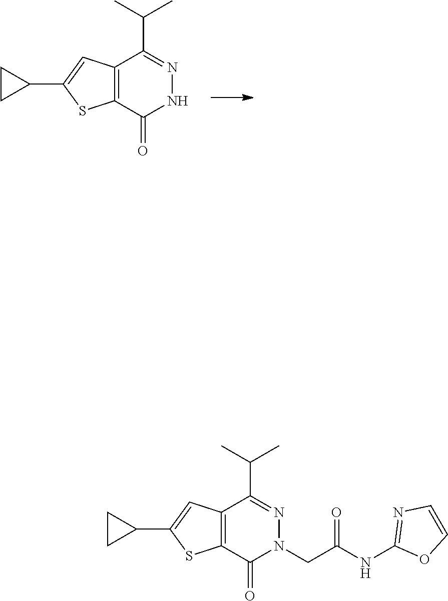 [SMILES: CC(C)c1n[nH]c(=O)c2sc(C3CC3)cc12 ; CC(C)c1nn(CC(=O)Nc2ncco2)c(=O)c2sc(C3CC3)cc12]

The reaction was performed in the same manner as compound 72, using 2-cyclopropyl-4-isopropyl-6H-thieno[2,3-d]pyridazin-7-one (Intermediate C37, 100 mg, 427 mol) and 2-chloro-N-oxazol-2-yl-acetamide (Intermediate B7, 137 mg, 854 mol) at 25° C. The crude material was purified by prep-HPLC (column: Waters Xbridge Prep OBD C18 150×40 mm, 10 μm; mobile phase: [(A) water (NH₄HCO₃)−(B) ACN]; B: 25-45%, 8 min) and lyophilised to give the title compound as a white solid, Y=12%. ¹H NMR (400 MHz, DMSO-d₆) δ 8.98 (br., 1H), 7.85 (s, 1H), 7.41 (s, 1H), 7.10 (s, 1H), 5.00 (s, 2H), 3.30-3.25 (m, 1H), 2.45-2.25 (m, 1H), 1.25 (d, J=7 Hz, 6H), 1.15-1.21 (m, 2H), 0.86-0.96 (m, 2H). LCMS (ESI): m/z [M+H]⁺=359.1.

### Compound 77. 2-(2-Cyclopropyl-4-isopropyl-7-oxofuro[2,3-d]pyridazin-6(7H)-yl)-N-(pyrimidin-2-yl)acetamide

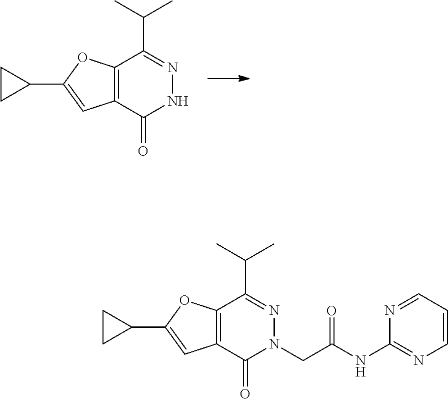 [SMILES: CC(C)c1n[nH]c(=O)c2cc(C3CC3)oc12 ; CC(C)c1nn(CC(=O)Nc2ncccn2)c(=O)c2cc(C3CC3)oc12]

The reaction was performed in the same manner as reaction 72, using 2-cyclopropyl-7-isopropyl-5H-furo[2,3-d]pyridazin-4-one (Intermediate C29, 50 mg, 229 mol) in DMF (2 mL) was added 2-chloro-N-pyrimidin-2-yl-acetamide (Intermediate B3, 79 mg, 458 mol). The crude material was purified by prep-HPLC (column: Waters Xbridge BEH C18 100×30 mm, 10 μm; mobile phase: [(A) water (NH₄HCO₃)−(B) ACN]; B: 25-50%, 8 min) and lyophilised to give the title compound as a white solid, Y=21%. ¹H NMR (400 MHz, DMSO-d₆) δ 10.91 (s, 1H), 8.67 (d, J=5 Hz, 2H), 7.20 (t, J=5 Hz, 1H), 6.80 (s, 1H), 5.15 (s, 2H), 3.27-3.16 (m, 1H), 2.24-2.14 (m, 1H), 1.29 (d, J=7 Hz, 6H), 1.12-1.04 (m, 2H), 0.99-0.93 (m, 2H). LCMS (ESI): m/z [M+H]⁺=354.1.

### Compound 78. 2-(2-(Ethylamino)-4-isopropyl-7-oxothieno[2,3-d]pyridazin-6(7H)-yl)-N-(pyrimidin-2-yl)acetamide

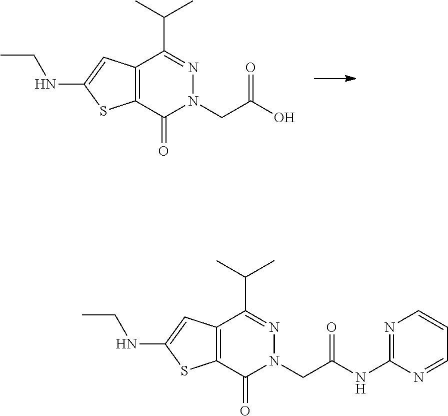 [SMILES: CCNc1cc2c(C(C)C)nn(CC(=O)O)c(=O)c2s1 ; CCNc1cc2c(C(C)C)nn(CC(=O)Nc3ncccn3)c(=O)c2s1]

To a solution of 2-[2-(ethylamino)-4-isopropyl-7-oxo-thieno[2,3-d]pyridazin-6-yl]acetic acid (Intermediate C39 100 mg, 339 μmol) in ACN (1 mL) at 25° C. under N₂ was added COMU (218 mg, 508 μmol), NMM (339 μmol, 37 μL) and pyrimidin-2-amine (48 mg, 508 μmol). The reaction mixture was stirred at 50° C. for 12 hr then it was diluted with H₂O (1 mL) and extracted with EtOAc (3×1 mL). The combined organic layers were washed with brine (1 mL), dried over anhydrous Na₂SO₄, filtered and concentrated under reduced pressure. The crude material was purified by prep-HPLC (column: Phenomenex C18 75×30 mm, 3 m; mobile phase: [(A) water (NH₄HCO₃)−(B) ACN]; B: 25-45%, 8 min) and lyophilised to give the title compound as a white solid, Y=17%. ¹H NMR (400 MHz, DMSO-d₆) δ 10.86 (br., 1H), 8.67 (d, J=5 Hz, 2H), 7.56 (t, J=5 Hz, 1H), 7.20 (t, J=5 Hz, 1H), 6.10 (s, 1H), 5.08 (s, 2H), 3.22-3.12 (m, 3H), 1.25-1.15 (m, 9H). LCMS (ESI): m/z [M+H]⁺=373.2.

### Compound 79. 2-(2-Cyclopropyl-7-isopropyl-4-oxofuro[2,3-d]pyridazin-5(4H)-yl)-N-(oxazol-2-yl)acetamide

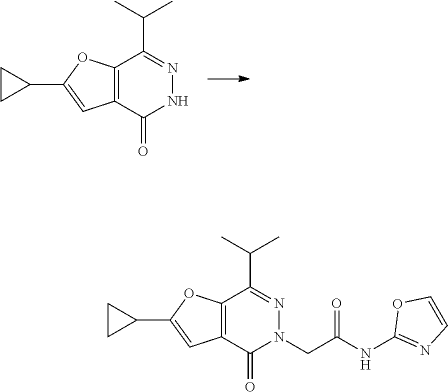 [SMILES: CC(C)c1n[nH]c(=O)c2cc(C3CC3)oc12 ; CC(C)c1nn(CC(=O)Nc2ncco2)c(=O)c2cc(C3CC3)oc12]

The reaction was performed in the same manner as compound 72, using 2-cyclopropyl-7-isopropyl-5H-furo[2,3-d]pyridazin-4-one (Intermediate C29, 50 mg, 229 mol) 2-chloro-N-oxazol-2-yl-acetamide (Intermediate B7, 74 mg, 458 mol). The crude material was purified by prep-HPLC (column: Waters Xbridge BEH C18 100×30 mm, 10 m; mobile phase: [(A) water (NH₄HCO₃)−(B) ACN]; B: 20-50%, 8 min) and lyophilised to give the title compound as a white solid, Y=23%. ¹H NMR (400 MHz, DMSO-d₆) δ 11.52 (s, 1H), 7.87 (s, 1H), 7.11 (s, 1H), 6.80 (s, 1H), 4.97 (s, 2H), 3.27-3.15 (m, 1H), 2.25-2.14 (m, 1H), 1.29 (d, J=7 Hz, 6H), 1.11-1.04 (m, 2H), 0.98-0.93 (m, 2H). LCMS (ESI): m/z [M+H]⁺=343.1.

### Compound 80. Ethyl 2-(2-(2-cyclopropyl-7-isopropyl-4-oxofuro[2,3-d]pyridazin-5(4H)-yl)acetamido)pyrimidine-5-carboxylate

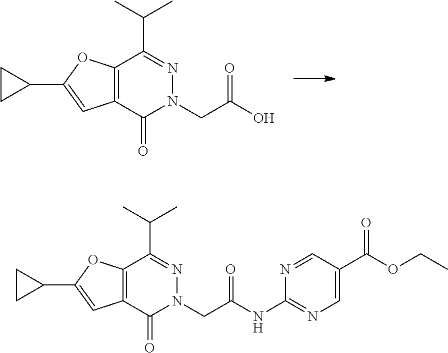 [SMILES: CC(C)c1nn(CC(=O)O)c(=O)c2cc(C3CC3)oc12 ; CCOC(=O)c1cnc(NC(=O)Cn2nc(C(C)C)c3oc(C4CC4)cc3c2=O)nc1]

The reaction was performed in the same manner as Compound 78, using 2-(2-cyclopropyl-7-isopropyl-4-oxo-furo[2,3-d]pyridazin-5-yl)acetic acid (55 mg, 199 mol) and ethyl 2-aminopyrimidine-5-carboxylate (50 mg, 299 mol). The crude material was purified by prep-HPLC (column: Waters Xbridge BEH C18 100×30 mm, 10 m; mobile phase: [(A) water (NH₄HCO₃)−(B) ACN]; B: 35-65%, 8 min) and lyophilised to give the title compound as a white solid, Y=11%. ¹H NMR (400 MHz, DMSO-d₆) δ 10.77 (br., 1H), 9.09 (s, 2H), 6.80 (s, 1H), 5.19 (s, 2H), 4.35 (q, J=7 Hz, 2H), 3.26-3.17 (m, 1H), 2.25-2.15 (m, 1H), 1.33 (t, J=7 Hz, 3H), 1.29 (d, J=7 Hz, 6H), 1.12-1.03 (m, 2H), 1.00-0.92 (m, 2H). LCMS (ESI): m/z [M+H]⁺=425.8.

### Compound 81. 2-(2-Cyclopropyl-7-isopropyl-4-oxofuro[2,3-d]pyridazin-5(4H)-yl)-N-((1s,3s)-3-hydroxy-3-methylcyclobutyl)acetamide

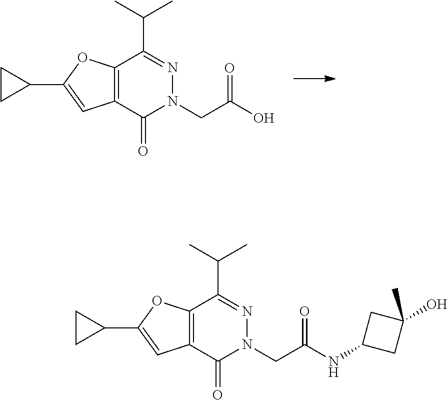 [SMILES: CC(C)c1nn(CC(=O)O)c(=O)c2cc(C3CC3)oc12 ; CC(C)c1nn(CC(=O)N[C@H]2C[C@@](C)(O)C2)c(=O)c2cc(C3CC3)oc12]

To a solution of 2-(2-cyclopropyl-7-isopropyl-4-oxo-furo[2,3-d]pyridazin-5-yl)acetic acid (Intermediate C38, 40 mg, 145 mol) in DMF (1 mL) at 0° C. was added HATU (83 mg, 217 mol) and the solution was stirred at 0° C. for 0.5 h. The mixture was allowed to warm to 25° C. then was added 3-amino-1-methyl-cyclobutanol hydrochloride (22 mg, 160 mol) and DIPEA (76 μL, 434 mol) and was stirred for 2 h. The reaction mixture was concentrated under reduced pressure. The crude material was purified by prep-HPLC (column: Waters Xbridge Prep OBD C18 150×40 mm, 10 m; mobile phase: [(A) water (NH₄HCO₃)−(B) ACN]; B: 20-50%, 8 min) and lyophilised to give the title compound as a white solid, Y=86%. ¹H NMR (400 MHz, DMSO-d₆) δ 8.22 (d, J=7 Hz, 1H), 6.77 (s, 1H), 4.98 (s, 1H), 4.63 (s, 2H), 3.81-3.67 (m, 1H), 3.24-3.12 (m, 1H), 2.27-2.14 (m, 3H), 1.98-1.87 (m, 2H), 1.27 (d, J=7 Hz, 6H), 1.20 (s, 3H), 1.11-1.04 (m, 2H), 0.97-0.91 (m, 2H). LCMS (ESI): m/z [M+H]⁺=360.2.

### Compound 82. 2-{2-Cyclopropyl-7-methyl-4-oxo-4H,5H-furo[2,3-d]pyridazin-5-yl}-N-(pyrimidin-2-yl)acetamide

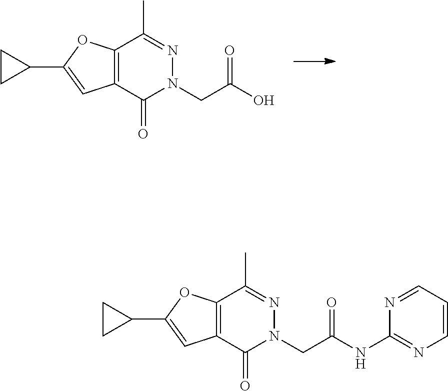 [SMILES: Cc1nn(CC(=O)O)c(=O)c2cc(C3CC3)oc12 ; Cc1nn(CC(=O)Nc2ncccn2)c(=O)c2cc(C3CC3)oc12]

To a solution of 2-(2-cyclopropyl-7-methyl-4-oxo-furo[2,3-d]pyridazin-5-yl)acetic acid (Intermediate D1, 200 mg, 0.81 mmol) in ACN (5 mL) was added pyrimidin-2-amine (115 mg, 1.21 mmol), COMU (518 mg, 1.21 mmol) and NMM (89 μL, 0.81 mmol) at 25° C. under N₂. The resulting mixture was stirred at 50° C. for 12 h. The solution was concentrated under vacuum. The residue was purified by prep-HPLC (column: Waters Xbridge Prep OBD C18 150×40 mm×10 μm; mobile phase: [water (NH₄HCO₃)−ACN]; B: 20-50%, 8 min) and lyophilised to give the title compound as a white solid. Y=42%. ¹H NMR (400 MHz, DMSO-d₆) δ 10.91 (s, 1H), 8.67 (d, J=5 Hz, 2H), 7.20 (t, J=5 Hz, 1H), 6.80 (s, 1H), 5.15 (s, 2H), 2.43 (s, 3H), 2.23-2.14 (m, 1H), 1.10-1.04 (m, 2H), 0.98-0.94 (m, 2H).

### Compound 83. 2-{2-Cyclopropyl-7-methyl-4-oxo-4H,5H-furo[2,3-d]pyridazin-5-yl}-N-(5-methylpyrimidin-2-yl)acetamide

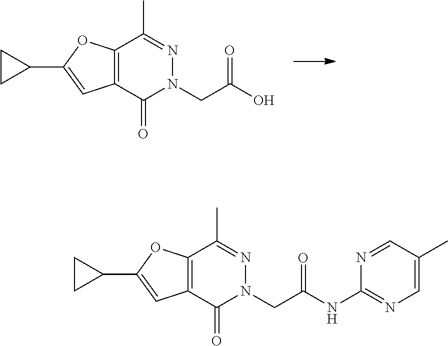 [SMILES: Cc1nn(CC(=O)O)c(=O)c2cc(C3CC3)oc12 ; Cc1cnc(NC(=O)Cn2nc(C)c3oc(C4CC4)cc3c2=O)nc1]

Prepared in an analogous way to Compound 82 using Intermediate D1 and 5-methylpyrimidin-2-amine to give the title compound as a white solid. Y=14%. ¹H NMR (400 MHz, DMSO-d₆) δ 10.75 (br. s, 0.5H), 8.52 (s, 2H), 6.80 (s, 1H), 5.11 (s, 2H), 2.43 (s, 3H), 2.22 (s, 3H), 2.18-2.17 (m, 1H), 1.09-1.04 (m, 2H), 0.98-0.93 (m, 2H). ¹H NMR (400 MHz, DMSO-d₆+D₂O) δ 8.50 (s, 2H), 6.75 (s, 1H), 5.09 (s, 2H), 2.42 (s, 3H), 2.21 (s, 3H), 2.19-2.18 (m, 1H), 1.09-1.05 (m, 2H), 0.96-0.92 (m, 2H). LCMS (ESI): m/z: [M+H]⁺=340.1.

### Compound 84. N-(5-Chloropyrimidin-2-yl)-2-{2-cyclopropyl-7-methyl-4-oxo-4H,5H-furo[2,3-d]pyridazin-5-yl}acetamide

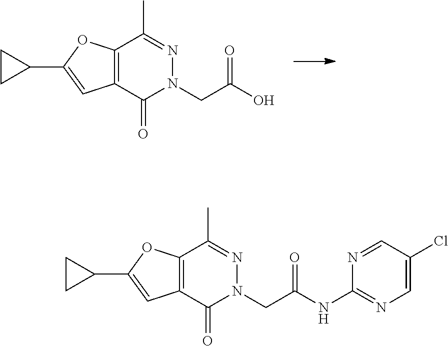 [SMILES: Cc1nn(CC(=O)O)c(=O)c2cc(C3CC3)oc12 ; Cc1nn(CC(=O)Nc2ncc(Cl)cn2)c(=O)c2cc(C3CC3)oc12]

Prepared in an analogous way to Compound 82 using Intermediate D1 and 5-chloropyrimidin-2-amine to give the title compound as a white solid. Y=12%. ¹H NMR (400 MHz, DMSO-d₆) δ 11.14 (s, 1H), 8.78 (s, 2H), 6.80 (s, 1H), 5.11 (s, 2H), 2.43 (s, 3H), 2.22-2.14 (m, 1H), 1.09-1.04 (m, 2H), 0.98-0.94 (m, 2H). ¹H NMR (400 MHz, DMSO-d₆+D₂O) δ 8.76 (s, 2H), 6.77 (s, 1H), 5.10 (s, 2H), 2.42 (s, 3H), 2.22-2.13 (m, 1H), 1.09-1.04 (m, 2H), 0.97-0.93 (m, 2H). LC-MS (ESI): m/z: [M+H]⁺=360.1.

### Compound 85. 2-{2-Cyclopropyl-7-methyl-4-oxo-4H,5H-furo[2,3-d]pyridazin-5-yl}-N-[cis-3-hydroxy-3-methylcyclobutyl]acetamide

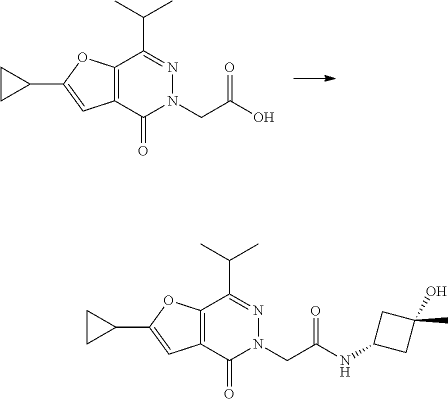 [SMILES: CC(C)c1nn(CC(=O)O)c(=O)c2cc(C3CC3)oc12 ; CC(C)c1nn(CC(=O)N[C@H]2C[C@@](C)(O)C2)c(=O)c2cc(C3CC3)oc12]

Prepared in an analogous way to Compound 82 using Intermediate D1 and cis-3-amino-1-methylcyclobutanol to give the title compound as a white solid. Y=56%. ¹H NMR (400 MHz, DMSO-d₆) δ 8.22 (d, J=7 Hz, 1H), 6.76 (s, 1H), 4.95 (s, 1H), 4.63 (s, 2H), 3.80-3.65 (m, 1H), 2.40 (s, 3H), 2.26-2.13 (m, 3H), 1.96-1.88 (m, 2H), 1.20 (s, 3H), 1.09-1.01 (m, 2H), 0.96-0.90 (m, 2H). ¹H NMR (400 MHz, DMSO-d₆+D₂O) δ 6.76 (s, 1H), 4.63 (s, 2H), 3.80-3.65 (m, 1H), 2.40 (s, 3H), 2.25-2.13 (m, 3H), 1.96-1.88 (m, 2H), 1.19 (s, 3H), 1.11-1.00 (m, 2H), 0.97-0.90 (m, 2H). LC-MS (ESI): m/z: [M+H]⁺=332.2.

### Compound 86. Ethyl 2-(2-{2-cyclopropyl-4-methyl-7-oxo-6H,7H-thieno[2,3-d]pyridazin-6-yl}acetamido)pyrimidine-5-carboxylate

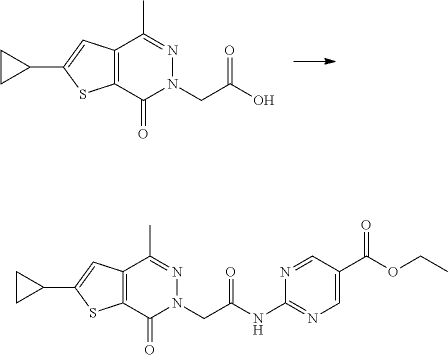 [SMILES: Cc1nn(CC(=O)O)c(=O)c2sc(C3CC3)cc12 ; CCOC(=O)c1cnc(NC(=O)Cn2nc(C)c3cc(C4CC4)sc3c2=O)nc1]

To a solution of 2-(2-cyclopropyl-4-methyl-7-oxo-thieno[2,3-d]pyridazin-6-yl)acetic acid (Intermediate D2, 100 mg, 378 μmol) in ACN (1 mL) was added ethyl 2-aminopyrimidine-5-carboxylate (94.9 mg, 568 μmol), COMU (243 mg, 568 μmol) and NMM (41.6 μL, 378 μmol) at 25° C. The mixture was stirred at 80° C. for 2 h. The reaction mixture was diluted with H₂O (1 mL) and extracted with EtOAc (3×1 mL). The combined organic layers were washed with brine (1 mL), dried over Na₂SO₄, filtered and concentrated under reduced pressure to give a residue. The residue was purified by prep-HPLC (column: Phenomenex C18 75×30 mm×3 μm; mobile phase: [water (NH₄HCO₃)−ACN]; B: 15-35%, 8 min) and lyophilised to give the title compound as a white solid. Y=3.2%. ¹H NMR (400 MHz, DMSO-d₆) δ 11.42 (s, 1H), 9.09 (s, 2H), 7.31 (s, 1H), 5.20 (s, 2H), 4.35 (q, J=7 Hz, 2H), 2.47 (s, 3H), 2.40-2.30 (m, 1H), 1.45 (t, J=7 Hz, 3H), 1.21-1.16 (m, 2H), 0.93-0.88 (m, 2H). ¹H NMR (400 MHz, DMSO-d₆+D₂O) δ 9.06 (s, 2H), 7.27 (s, 1H), 5.16 (s, 2H), 4.41-4.27 (m, 2H), 2.46-2.31 (m, 4H), 1.41-1.27 (m, 3H), 1.25-1.15 (m, 2H), 0.92-0.86 (m, 2H). LCMS (ESI): m/z [M+H]⁺=414.1.

### Compound 87. 2-{2-cyclopropyl-7-methyl-4-oxo-4H,5H-furo[2,3-d]pyridazin-5-yl}-N-(1,3-oxazol-2-yl)acetamide

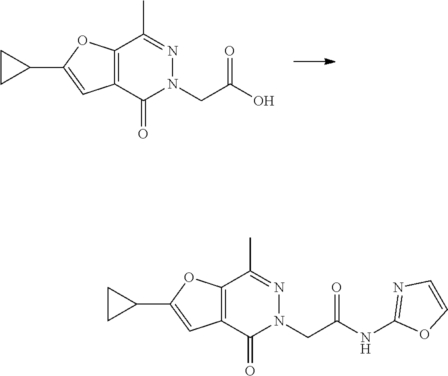 [SMILES: Cc1nn(CC(=O)O)c(=O)c2cc(C3CC3)oc12 ; Cc1nn(CC(=O)Nc2ncco2)c(=O)c2cc(C3CC3)oc12]

Prepared in an analogous way to Compound 82 using Intermediate D1 and 2-aminooxazole to give the title compound as a white solid. Y=26%. ¹H NMR (400 MHz, DMSO-d₆) δ 11.54 (br. s, 1H), 7.87 (s, 1H), 7.11 (s, 1H), 6.80 (s, 1H), 4.97 (s, 2H), 2.43 (s, 3H), 2.23-2.13 (m, 1H), 1.11-1.02 (m, 2H), 0.99-0.92 (m, 2H). ¹H NMR (400 MHz, DMSO-d₆+D₂O) δ 7.80 (s, 1H), 7.08 (s, 1H), 6.75 (s, 1H), 4.95 (s, 2H), 2.41 (s, 3H), 2.22-2.13 (m, 1H), 1.10-1.02 (m, 2H), 0.97-0.90 (m, 2H). LC-MS (ESI): m/z: [M+H]⁺=315.1.

### Compound 88. 2-{2-Cyclopropyl-4-methyl-7-oxo-6H,7H-thieno[2,3-d]pyridazin-6-yl}-N-(pyrimidin-2-yl)acetamide

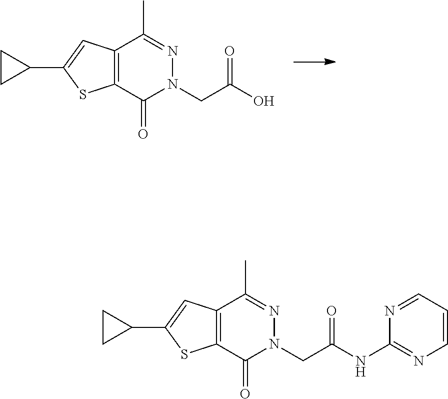 [SMILES: Cc1nn(CC(=O)O)c(=O)c2sc(C3CC3)cc12 ; Cc1nn(CC(=O)Nc2ncccn2)c(=O)c2sc(C3CC3)cc12]

Prepared in an analogous way to Compound 86 using Intermediate D2 and 2-aminopyrimidine to give the title compound as a white solid. Y=9.6%. ¹H NMR (400 MHz, DMSO-d₆) δ 10.94 (s, 1H), 8.67 (d, J=5 Hz, 2H), 7.31 (s, 1H), 7.20 (t, J=5 Hz, 1H), 5.15 (s, 2H), 2.46 (s, 3H), 2.40-2.30 (m, 1H), 1.22-1.14 (m, 2H), 0.95-0.88 (m, 2H). ¹H NMR (400 MHz, DMSO-d₆+D₂O) δ 8.62 (d, J=5 Hz, 2H), 7.24 (s, 1H), 7.18 (t, J=5 Hz, 1H), 5.09 (s, 2H), 2.43 (s, 3H), 2.38-2.26 (m, 1H), 1.20-1.13 (m, 2H), 0.91-0.81 (m, 2H). LCMS (ESI): m/z: [M+H]⁺=342.1.

### Compound 89. 2-{2-cyclopropyl-7-ethyl-4-oxo-4H,5H-furo[2,3-d]pyridazin-5-yl}-N-(pyrimidin-2-yl)acetamide

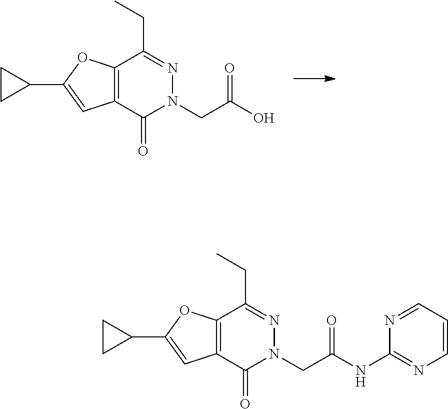 [SMILES: CCc1nn(CC(=O)O)c(=O)c2cc(C3CC3)oc12 ; CCc1nn(CC(=O)Nc2ncccn2)c(=O)c2cc(C3CC3)oc12]

Prepared in an analogous way to Compound 82 using Intermediate D3 and pyrimidin-2-amine aminopyrimidine to give the title compound as a white solid. Y=52%. ¹H NMR (400 MHz, DMSO-d₆) δ 10.95 (br. s, 1H), 8.67 (d, J 5 Hz, 2H), 7.20 (t, J 5 Hz, 1H), 6.80 (s, 1H), 5.15 (s, 2H), 2.82 (q, J 8 Hz, 2H), 2.25-2.12 (m, 1H), 1.25 (t, J 8 Hz, 3H), 1.10-1.04 (m, 2H), 0.99-0.93 (m, 2H). ¹H NMR (400 MHz, DMSO-d₆+D₂O) δ 8.65 (d, J 5 Hz, 2H), 7.19 (t, J 5 Hz, 1H), 6.77 (s, 1H), 5.13 (s, 2H), 2.81 (q, J 8 Hz, 2H), 2.23-2.12 (m, 1H), 1.23 (t, J 8 Hz, 3H), 1.10-1.04 (m, 2H), 0.97-0.92 (m, 2H). LC-MS (ESI): m/z: [M+H]⁺=340.1.

### Compound 90. Ethyl 2-(2-{2-cyclopropyl-7-methyl-4-oxo-4H,5H-furo[2,3-d]pyridazin-5-yl}acetamido)pyrimidine-5-carboxylate

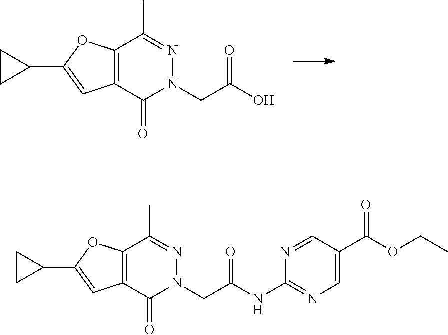 [SMILES: Cc1nn(CC(=O)O)c(=O)c2cc(C3CC3)oc12 ; CCOC(=O)c1cnc(NC(=O)Cn2nc(C)c3oc(C4CC4)cc3c2=O)nc1]

Prepared in an analogous way to Compound 82 using Intermediate D1 and ethyl 2-aminopyrimidine-5-carboxylate to give the title compound as a white solid. Y=10%. ¹H NMR (400 MHz, DMSO-d₆) δ 11.38 (s, 1H), 9.09 (s, 2H), 6.81 (s, 1H), 5.19 (s, 2H), 4.35 (q, J=7 Hz, 2H), 2.44 (s, 3H), 2.23-2.11 (m, 1H), 1.33 (t, J=7 Hz, 3H), 1.10-1.00 (m, 2H), 0.99-0.94 (m, 2H). ¹H NMR (400 MHz, DMSO-d₆+D₂O) δ 9.06 (s, 2H), 6.73 (s, 1H), 5.15 (s, 2H), 4.31 (q, J=7 Hz, 2H), 2.41 (s, 3H), 2.22-2.08 (m, 1H), 1.30 (t, J=7 Hz, 3H), 1.09-1.02 (m, 2H), 0.96-0.90 (m, 2H). LCMS (ESI): m/z: [M+H]⁺=398.2.

### Compound 91. N-(5-Cyanopyrimidin-2-yl)-2-{2-cyclopropyl-7-methyl-4-oxo-4H,5H-furo[2,3-d]pyridazin-5-yl}acetamide

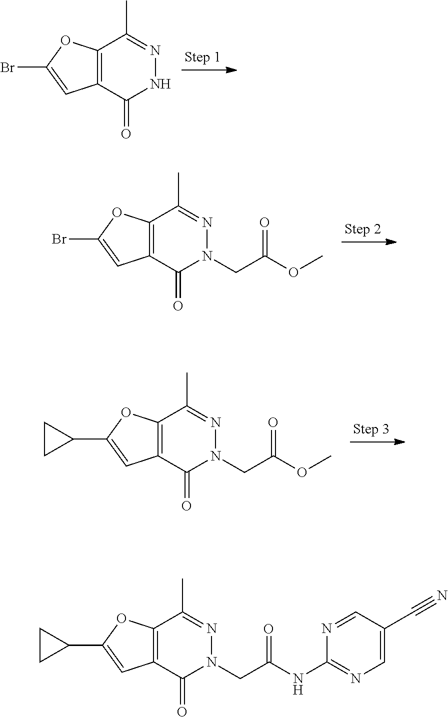 [SMILES: Cc1n[nH]c(=O)c2cc(Br)oc12 ; COC(=O)Cn1nc(C)c2oc(Br)cc2c1=O ; COC(=O)Cn1nc(C)c2oc(C3CC3)cc2c1=O ; Cc1nn(CC(=O)Nc2ncc(C#N)cn2)c(=O)c2cc(C3CC3)oc12]

Step 1. Methyl 2-(2-bromo-7-methyl-4-oxo-furo[2,3-d]pyridazin-5-yl)acetate. To a solution of 2-bromo-7-methyl-5H-furo[2,3-d]pyridazin-4-one (for synthesis see Intermediate D1)(170 mg, 0.74 mmol) in DMF (2 mL) was added Cs₂CO₃ (726 mg, 2.23 mmol) and methyl 2-bromoacetate (77.1 μL, 0.82 mmol) at 25° C. The mixture was stirred at 80° C. for 1 h. The reaction mixture was diluted with H₂O (2 mL) and extracted with ethyl acetate (3×2 mL). The combined organic layers were washed with brine (2 mL), dried over anhydrous Na₂SO₄, filtered and concentrated under reduced pressure to give the title compound as a yellow oil. Y=98%.

Step 2. Methyl 2-(2-cyclopropyl-7-methyl-4-oxo-furo[2,3-d]pyridazin-5-yl)acetate. To a solution of methyl 2-(2-bromo-7-methyl-4-oxo-furo[2,3-d]pyridazin-5-yl)acetate (220 mg, 0.73 mmol) in toluene (5 mL) and H₂O (0.5 mL) was added cyclopropylboronic acid (94 mg, 1.10 mmol), Pd(OAc)₂ (8.2 mg, 37 μmol), tricyclohexylphosphine (20.5 mg, 73 μmol) and K₃PO₄ (543 mg, 2.56 mmol) at 25° C. under N₂. The resulting mixture was stirred at 80° C. under N₂ for 12 h. The reaction mixture was diluted with H₂O (5 mL) and extracted with ethyl acetate (3×5 mL). The combined organic layers were washed with brine (5 mL), dried over anhydrous Na₂SO₄, filtered and concentrated under reduced pressure to give a residue. The residue was purified by column chromatography (10-50% EtOAc in petroleum ether) to give the title compound as a yellow oil. Y=52%. ¹H NMR (400 MHz, DMSO-d₆) δ 6.80 (s, 1H), 4.88 (s, 2H), 3.67 (s, 3H), 2.43 (s, 3H), 2.23-2.14 (m, 1H), 1.10-1.03 (m, 2H), 0.99-0.93 (m, 2H).

Step 3. N-(5-Cyanopyrimidin-2-yl)-2-{2-cyclopropyl-7-methyl-4-oxo-4H,5H-furo[2,3-d]pyridazin-5-yl}acetamide. To a solution of 2-aminopyrimidine-5-carbonitrile (115 mg, 0.95 mmol) in toluene (2 mL) was added Al(CH₃)₃ (2 M in toluene, 0.95 mL) at 0° C. The mixture was stirred for 1 h, then the mixture was treated with methyl 2-(2-cyclopropyl-7-methyl-4-oxo-furo[2,3-d]pyridazin-5-yl)acetate (100 mg, 0.38 mmol) at 0° C. The resulting mixture was stirred at 25° C. for 12 h. The mixture was quenched by saturated aqueous NH₄Cl solution (2 mL) and extracted with EtOAC (3×2 mL). The combined organic layers were washed with brine (2 mL), dried over anhydrous Na₂SO₄, filtered and concentrated under reduced pressure to give a residue. The residue was purified by prep-HPLC (column: Waters Xbridge Prep OBD C18 150×40 mm×10 μm; mobile phase: [water (NH₄HCO₃)−ACN]; B: 20-45%, 8 min) and lyophilised to give the title compound as a white solid. Y=27%. ¹H NMR (400 MHz, DMSO-d₆) δ 9.09 (s, 2H), 6.80 (s, 1H), 5.16 (s, 2H), 2.43 (s, 3H), 2.23-2.13 (m, 1H), 1.09-1.04 (m, 2H), 0.98-0.94 (m, 2H). LCMS (ESI): m/z: [M+H]⁺=351.0.

### Compound 92. Ethyl 2-[2-(2-{2-cyclopropyl-7-methyl-4-oxo-4H,5H-furo[2,3-d]pyridazin-5-yl}acetamido)pyrimidin-5-yl]acetate

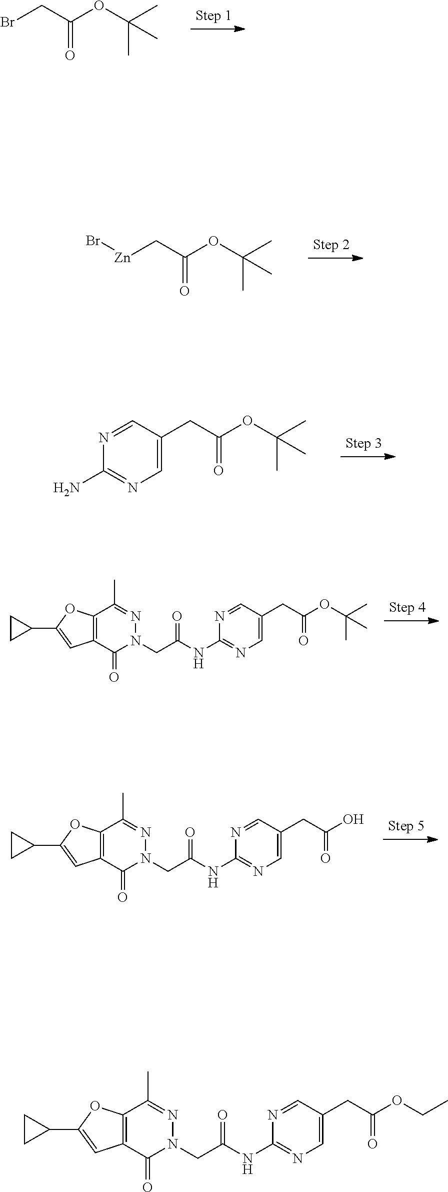

Step 1. Bromo-(2-tert-butoxy-2-oxo-ethyl)zinc. To a suspension of Zn (53 g, 0.81 mmol) in THE (1050 mL) was added TMSCl (6.83 mL, 53.8 mmol) at 20° C. under N₂. The mixture was stirred at 25° C. for 1 h. To the mixture was added tert-butyl 2-bromoacetate (53.0 mL, 359 mmol) at 25° C. under N₂. The mixture was stirred at 60° C. for 2 h under N₂ to give the title compound as a 0.34 M solution in THF. Y=quantitative.

Step 2. Tert-butyl 2-(2-aminopyrimidin-5-yl)acetate. To a solution of 5-bromopyrimidin-2-amine (6.0 g, 34.5 mmol) in THE (180 mL) was added Pd₂(dba)₃ (1.58 g, 1.72 mmol) and XPhos (1.64 g, 3.45 mmol) at 25° C. under N₂. The mixture was stirred at 25° C. for 1 h, then treated with bromo-(2-tert-butoxy-2-oxo-ethyl)zinc (0.34 M in THF, 609 mL) at 25° C. under N₂. The mixture was stirred at 80° C. for 1 h under N₂. The reaction mixture was quenched by addition of saturated NH₄Cl aqueous solution (80 mL) at 0° C., and extracted with EtOAc (3×30 mL). The combined organic layers were washed with brine (2×20 mL), dried over anhydrous Na₂SO₄, filtered and concentrated under reduced pressure to give a residue. The residue was purified by prep-HPLC (column: Welch Xtimate C₁₈ 250×100 mm×10 μm; mobile phase: [water (NH₄HCO₃)−ACN]; B: 15-45%, 18 min) to give the title compound as a white solid. Y=35%. ¹H NMR (400 MHz, DMSO-d₆) δ 8.09 (s, 2H), 6.50 (s, 2H), 3.37 (s, 2H), 1.40 (s, 9H).

Step 3. Tert-butyl 2-[2-[[2-(2-cyclopropyl-7-methyl-4-oxo-furo[2,3-d]pyridazin-5-yl)acetyl]amino]pyrimidin-5-yl]acetate. To a solution of 2-(2-cyclopropyl-7-methyl-4-oxo-furo[2,3-d]pyridazin-5-yl)acetic acid (130 mg, 0.52 mmol) in ACN (4 mL) was added tert-butyl 2-(2-aminopyrimidin-5-yl)acetate (164 mg, 0.79 mmol), COMU (336 mg, 0.79 mmol) and NMM (58 μL, 0.52 mmol) at 25° C. The resulting mixture was stirred at 50° C. for 12 h under N₂. The solution was concentrated under vacuum. The residue was purified by column chromatography (10-30% EtOAc in petroleum ether) to give the title compound as a yellow oil. Y=87%. ¹H NMR (400 MHz, DMSO-d₆) δ 10.90 (s, 1H), 8.56 (s, 2H), 6.80 (s, 1H), 5.13 (s, 2H), 3.62 (s, 2H), 2.43 (s, 3H), 2.20-2.16 (m, 1H), 1.41 (s, 9H), 1.10-1.04 (m, 2H), 0.99-0.94 (m, 2H).

Step 4. 2-[2-[[2-(2-cyclopropyl-7-methyl-4-oxo-furo[2,3-d]pyridazin-5-yl)acetyl]amino]pyrimidin-5-yl]acetic acid. To a solution of tert-butyl 2-[2-[[2-(2-cyclopropyl-7-methyl-4-oxo-furo[2,3-d]pyridazin-5-yl) acetyl]amino]pyrimidin-5-yl]acetate (180 mg, 0.41 mmol) in DCM (2 mL) was added TFA (2 mL) and the mixture was stirred at 25° C. for 2 h. The reaction mixture was concentrated under vacuum to give the title compound as a yellow solid. Y=99%.

Step 5. Ethyl 2-[2-(2-{2-cyclopropyl-7-methyl-4-oxo-4H,5H-furo[2,3-d]pyridazin-5-yl}acetamido)pyrimidin-5-yl]acetate. To a solution of 2-[2-[[2-(2-cyclopropyl-7-methyl-4-oxo-furo[2,3-d]pyridazin-5-yl)acetyl]amino]pyrimidin-5-yl]acetic acid (155 mg, 0.40 mmol) in EtOH (3 mL) was added SOCl₂ (59 μL, 0.81 mmol) at 0° C. The mixture was stirred at 25° C. for 2 h, then concentrated under vacuum. The residue was purified by prep-HPLC (column: Waters Xbridge Prep OBD C18 150×40 mm×10 μm; mobile phase: [water (NH₄HCO₃)-ACN]; B: 25-55%, 8 min) and lyophilised to give the title compound as a white solid. Y=26%. ¹H NMR (400 MHz, DMSO-d₆) δ 11.70 (br. s, 0.4H), 8.58 (s, 2H), 6.80 (s, 1H), 5.13 (s, 2H), 4.11 (q, J=7 Hz, 2H), 3.73 (s, 2H), 2.43 (s, 3H), 2.23-2.14 (m, 1H), 1.20 (t, J=7 Hz, 3H), 1.09-1.04 (m, 2H), 0.98-0.94 (m, 2H). ¹H NMR (400 MHz, DMSO-d₆+D₂O) δ 8.55 (s, 2H), 6.75 (s, 1H), 5.10 (s, 2H), 4.09 (q, J=7 Hz, 2H), 3.70 (s, 2H), 2.42 (s, 3H), 2.21-2.13 (m, 1H), 1.18 (t, J=7 Hz, 3H), 1.09-1.03 (m, 2H), 0.97-0.91 (m, 2H). LCMS (ESI): m/z: [M+H]⁺=412.0.

### Compound 93. 2-[7-Oxo-4-(propan-2-yl)-2-[(propan-2-yl)amino]-6H,7H-thieno[2,3-d]pyridazin-6-yl]-N-(pyrimidin-2-yl)acetamide

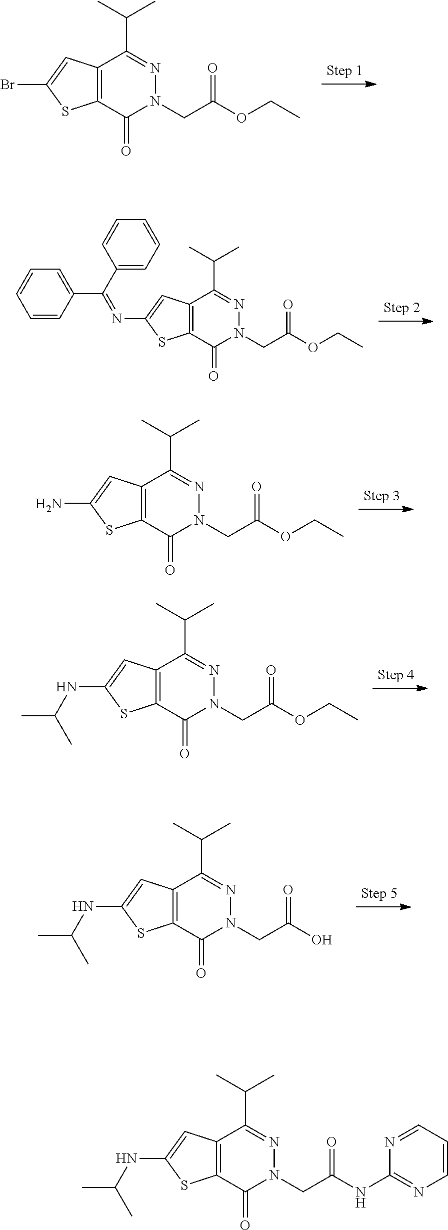

Step 1. Ethyl 2-[2-(benzhydrylideneamino)-4-isopropyl-7-oxo-thieno[2,3-d]pyridazin-6-yl]acetate. To a solution of ethyl 2-(2-bromo-4-isopropyl-7-oxo-thieno[2,3-d]pyridazin-6-yl)acetate (Intermediate D4, 0.70 g, 1.95 mmol) in dioxane (7 mL) was added Pd(OAc)₂ (44 mg, 0.19 mmol), diphenylmethanimine (392 μL 2.34 mmol), (5-diphenylphosphanyl-9,9-dimethyl-xanthen-4-yl)-diphenyl-phosphane (113 mg, 0.20 mmol) and Cs₂CO₃ (1.27 g, 3.90 mmol) at 25° C. The mixture was stirred at 105° C. for 12 h under N₂. The reaction mixture was diluted with H₂O (10 mL) and extracted with DCM (3×10 mL). The combined organic layers were washed with brine (3×10 mL), dried over Na₂SO₄, filtered and concentrated under reduced pressure to give a residue. The residue was purified by column chromatography (10-25% EtOAc in petrol) to give the title compound as a yellow solid. Y=89%. ¹H NMR (400 MHz, DMSO-d₆) δ 7.80-7.70 (m, 2H), 7.65-7.55 (m, 4H), 7.54-7.50 (m 2H), 7.45-7.32 (m, 3H), 4.82 (s, 2H), 4.12 (q, J=7 Hz, 2H), 3.30-3.22 (m, 1H), 1.25-1.15 (m, 9H).

Step 2. Ethyl 2-{2-[(diphenylmethylidene)amino]-7-oxo-4-(propan-2-yl)-6H,7H-thieno[2,3-d]pyridazin-6-yl}acetate. To a solution of ethyl 2-[2-(benzhydrylideneamino)-4-isopropyl-7-oxo-thieno[2,3-d]pyridazin-6-yl]acetate (0.20 g, 0.39 mmol) in MeOH (2 mL) was added hydroxylamine hydrochloride (93 mg, 1.34 mmol) and sodium acetate (97 mg, 1.18 mmol) at 25° C. The mixture was stirred at 25° C. for 2 h. The reaction mixture was concentrated under reduced pressure. The residue was diluted with H₂O (1 mL) and extracted with DCM (3×1 mL). The combined organic layers were washed with brine (1 mL), dried over Na₂SO₄, filtered and concentrated under reduced pressure to give a residue. The residue was purified by prep-HPLC (column: Waters Xbridge Prep OBD C18 150×40 mm×10 μm; mobile phase: [water (NH₄HCO₃)−ACN]; gradient: 20-50% over 8 min) and lyophilised to give the title compound as a white solid. Y=25%. ¹H NMR (400 MHz, DMSO-d₆) δ 10.87 (s, 1H), 8.67 (d, J=5 Hz, 2H), 7.20 (t, J=5 Hz, 1H), 6.99 (d, J=5 Hz, 1H), 6.10 (s, 1H), 5.07 (s, 2H), 3.15-3.05 (m, 1H), 1.21 (d, J=7 Hz, 6H). ¹H NMR (400 MHz, DMSO-d₆+D₂O) δ 8.64 (d, J=5 Hz, 2H), 7.18 (t, J=5 Hz, 1H), 6.14 (s, 1H), 5.04 (s, 2H), 3.20-2.95 (m, 1H), 1.20 (d, J=7 Hz, 6H). LCMS (ESI): m/z: [M+H]⁺=345.2.

Step 3. Ethyl 2-[4-isopropyl-2-(isopropylamino)-7-oxo-thieno[2,3-d]pyridazin-6-yl]acetate. To a solution of ethyl 2-(2-amino-4-isopropyl-7-oxo-thieno[2,3-d]pyridazin-6-yl)acetate (300 mg, 1.02 mmol) in DCM (3 mL) at 25° C. under N₂ were added 2-methoxypropene (439 mg, 6.09 mmol), sodium triacetoxyborohydride (646 mg, 3.05 mmol) and AcOH (290 μL, 5.08 mmol). The mixture was stirred at 25° C. for 2 h. The mixture was diluted with H₂O (5 mL) and the resulting mixture extracted with EtOAc (3×5 mL). The combined organic layers were washed with brine (5 mL), dried over Na₂SO₄, filtered and concentrated under reduced pressure to give a residue. The residue was purified by column chromatography (25-50% EtOAc in petrol) to give the title compound as a white solid. Y=76%.

Step 4. 2-[4-Isopropyl-2-(isopropylamino)-7-oxo-thieno[2,3-d]pyridazin-6-yl]acetic acid. To a solution of ethyl 2-[4-isopropyl-2-(isopropylamino)-7-oxo-thieno[2,3-d]pyridazin-6-yl]acetate (260 mg, 0.77 mmol) in THE (2 mL) and H₂O (2 mL) at 25° C. under N₂ was added LiOH·H₂O (65 mg, 1.54 mmol). The mixture was stirred at 25° C. for 1 h. The mixture was adjusted to pH 4 with 2 M HCl and the resulting mixture extracted with EtOAc (3×5 mL). The combined organic layers were washed with brine (5 mL), dried over Na₂SO₄, filtered and concentrated under reduced pressure to give the title compound as a white solid. Y=84%. ¹H NMR (400 MHz, DMSO-d₆) δ 12.79 (br. s, 1H), 7.50 (d, J=8 Hz, 1H), 6.08 (s, 1H), 4.70 (s, 2H), 3.65-3.55 (m, 1H), 3.20-3.07 (m, 1H), 1.21 (t, J=7 Hz, 12H).

Step 5. 2-[7-Oxo-4-(propan-2-yl)-2-[(propan-2-yl)amino]-6H,7H-thieno[2,3-d]pyridazin-6-yl]-N-(pyrimidin-2-yl)acetamide. To a solution of 2-[4-isopropyl-2-(isopropylamino)-7-oxo-thieno[2,3-d]pyridazin-6-yl]acetic acid (150 mg, 0.48 mmol) in ACN (2 mL) at 25° C. was added pyrimidin-2-amine (138 mg, 1.45 mmol), NMM (53 μL 0.48 mmol) and COMU (415 mg, 0.97 mmol). The mixture was stirred at 50° C. for 12 h. The mixture was diluted with H₂O (2 mL) and extracted with ethyl acetate (3×2 mL). The combined organic layers were washed with brine (2 mL), dried over Na₂SO₄, filtered and concentrated under reduced pressure to give a residue. The residue was purified by prep-HPLC (column: Waters Xbridge Prep OBD C₁₈ 150×40 mm×10 μm; mobile phase: [water (NH₄HCO₃)−ACN]; B: 25-55%, 8 min) and lyophilised to give the title compound as a white solid. Y=24%. ¹H NMR (400 MHz, DMSO-d₆) δ 10.86 (s, 1H), 8.67 (d, J=5 Hz, 2H), 7.50 (d, J=8 Hz, 1H), 7.20 (t, J=5 Hz, 1H), 6.10 (s, 1H), 5.07 (s, 2H), 3.62-3.56 (m, 1H), 3.19-3.10 (m, 1H), 1.21 (t, J=6 Hz, 12H). ¹H NMR (400 MHz, DMSO-d₆+D₂O) δ 8.66 (d, J=5 Hz, 2H), 7.19 (t, J=5 Hz, 1H), 6.09 (s, 1H), 5.06 (s, 2H), 3.62-3.55 (m, 1H), 3.18-3.09 (m, 1H), 1.20 (t, J=7 Hz, 12H). LCMS (ESI): m/z: [M+H]⁺=387.2.

### Compound 94. 2-[2-(Methylamino)-7-oxo-4-(propan-2-yl)-6H,7H-thieno[2,3-d]pyridazin-6-yl]-N-(pyrimidin-2-yl)acetamide

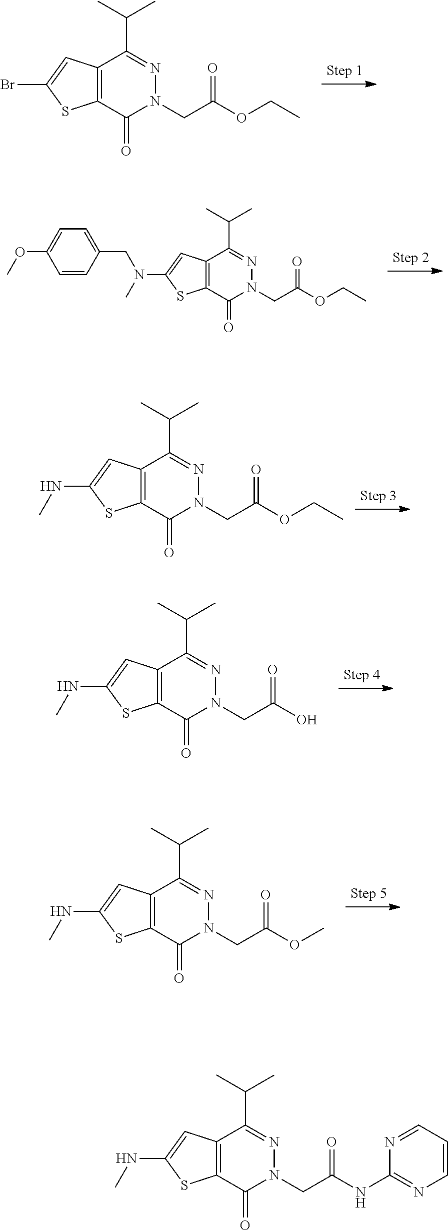

Step 1. Ethyl 2-[4-isopropyl-2-[(4-methoxyphenyl)methyl-methyl-amino]-7-oxo-thieno[2,3-d]pyridazin-6-yl]acetate. To a solution of ethyl 2-(2-bromo-4-isopropyl-7-oxo-thieno[2,3-d]pyridazin-6-yl)acetate (Intermediate D4, 500 mg, 1.39 mmol) in dioxane (5 mL) at 25° C. was added BINAP (87 mg, 139 μmol), 1-(4-methoxyphenyl)-N-methyl-methanamine (631 mg, 4.18 mmol), Cs₂CO₃ (680 mg, 2.09 mmol) and Pd(OAc)₂ (31 mg, 139 μmol). The mixture was stirred at 105° C. for 4 h. The mixture was diluted with H₂O (6 mL) and the resulting mixture extracted with ethyl acetate (3×6 mL). The combined organic layers were washed with brine (6 mL), dried over Na₂SO₄, filtered and concentrated under reduced pressure to give a residue. The residue was purified by prep-HPLC (column: Waters Xbridge Prep OBD C18 150×40 mm×10 μm; mobile phase: [water (NH₄HCO₃)−ACN]; B: 15-45%, 8 min) and lyophilised to give the title compound as a white solid. Y=87%. ¹H NMR (400 MHz, DMSO-d₆) δ 7.32-7.16 (m, 2H), 7.02-6.85 (m, 2H), 6.31 (s, 1H), 4.80 (s, 2H), 4.59 (s, 2H), 4.13 (d, J=7 Hz, 2H), 3.73 (s, 3H), 3.22-3.16 (m, 1H), 3.10 (s, 3H), 1.29-1.13 (m, 9H).

Step 2. Ethyl 2-[4-isopropyl-2-(methylamino)-7-oxo-thieno[2,3-d]pyridazin-6-yl]acetate. A mixture of ethyl 2-[4-isopropyl-2-[(4-methoxyphenyl)methyl-methyl-amino]-7-oxo-thieno [2,3-d]pyridazin-6-yl]acetate (300 mg, 0.70 mmol) and TFA (3 mL) was stirred at 70° C. for 12 h. The reaction mixture was diluted with H₂O (3 mL) and extracted with EtOAc (3×3 mL). The combined organic layers were washed with brine (3 mL), dried over anhydrous Na₂SO₄, filtered and concentrated under reduced pressure to give a residue. The residue was purified by column chromatography (10% EtOAc in petrol) to give the title compound as a white solid. Y=97%. ¹H NMR (400 MHz, DMSO-d₆) δ 7.59 (d, J=5 Hz, 1H), 6.09 (s, 1H), 4.79 (s, 2H), 4.14 (q, J=7 Hz, 2H), 3.20-3.10 (m, 1H), 2.86 (d, J=5 Hz, 3H), 1.22 (d, J=7 Hz, 6H), 1.20-1.15 (m, 3H).

Step 3. 2-[4-Isopropyl-2-(methylamino)-7-oxo-thieno[2,3-d]pyridazin-6-yl]acetic acid. To a solution of ethyl 2-[4-isopropyl-2-(methylamino)-7-oxo-thieno[2,3-d]pyridazin-6-yl]acetate (200 mg, 0.65 mmol) in THE (1 mL) and H₂O (1 mL) at 25° C. was added LiOH·H₂O (54 mg, 1.29 mmol). The mixture was stirred at 25° C. for 4 h. The reaction mixture was diluted with H₂O (3 mL) and extracted with EtOAc (3×3 mL). The combined organic layers were washed with brine (3 mL), dried over anhydrous Na₂SO₄, filtered and concentrated under reduced pressure to give a residue. The residue was purified by column chromatography (10% EtOAc in petrol) to give the title compound as a white solid. Y=93%. ¹H NMR (400 MHz, DMSO-d₆) δ 12.87 (s, 1H), 7.70-7.31 (m, 1H), 6.08 (s, 1H), 4.70 (s, 2H), 3.15 (q, J=7 Hz, 1H), 2.85 (d, J=5 Hz, 3H), 1.23 (d, J=7 Hz, 6H).

Step 4. Methyl 2-[4-isopropyl-2-(methylamino)-7-oxo-thieno [2,3-d]pyridazin-6-yl]acetate. To a solution of 2-[4-isopropyl-2-(methylamino)-7-oxo-thieno[2,3-d]pyridazin-6-yl]acetic acid (140 mg, 0.50 mmol) in MeOH (1.5 mL) at 25° C. was added TMSCl (6.3 uL, 50 μmol). The mixture was stirred at 50° C. for 4 h. The reaction mixture was diluted with H₂O (2 mL) and extracted with EtOAc (3×2 mL). The combined organic layers were washed with brine (2 mL), dried over anhydrous Na₂SO₄, filtered and concentrated under reduced pressure to give the title compound as a white solid. Y=95%.

Step 5. 2-[2-(Methylamino)-7-oxo-4-(propan-2-yl)-6H, 7H-thieno[2,3-d]pyridazin-6-yl]-N-(pyrimidin-2-yl)acetamide. To a solution of methyl 2-[4-isopropyl-2-(methylamino)-7-oxo-thieno[2,3-d]pyridazin-6-yl]acetate (140 mg, 0.47 mmol) in THE (1.5 mL) at 25° C. was added pyrimidin-2-amine (68 mg, 0.71 mmol). The mixture was cooled to 0° C. and treated with LiHMDS (1 M in THF, 1.04 mL, 1.04 mmol). The mixture was stirred at 25° C. for 12 h. The reaction mixture was diluted with H₂O (2 mL) and extracted with EtOAc (3×2 mL). The combined organic layers were washed with brine (2 mL), dried over anhydrous Na₂SO₄, filtered and concentrated under reduced pressure to give a residue. The residue was purified by prep-HPLC (column: Waters Xbridge Prep OBD C18 150×40 mm×10 μm; mobile phase: [water (NH₄HCO₃)−ACN]; B %: 15-40%, 8 min) and lyophilised to give the title compound as a white solid. Y=29%. ¹H NMR (400 MHz, DMSO-d₆) δ 10.87 (s, 1H), 8.67 (d, J=5 Hz, 2H), 7.56 (q, J=5 Hz, 1H), 7.20 (t, J=5 Hz, 1H), 6.12 (s, 1H), 5.08 (s, 2H), 3.20-3.10 (m, 1H), 2.86 (d, J=5 Hz, 3H), 1.23 (d, J=7 Hz, 6H). ¹H NMR (400 MHz, DMSO-d₆+D₂O) δ 8.64 (d, J=5 Hz, 2H), 7.18 (t, J=5 Hz, 1H), 6.09 (s, 1H), 5.05 (s, 2H), 3.20-3.10 (m, 1H), 2.83 (s, 3H), 1.20 (d, J=7 Hz, 6H). LCMS (ESI): m/z: [M+H]⁺=359.2.

### Compound 95. 2-{2-[(2,2-Difluoroethyl)amino]-7-oxo-4-(propan-2-yl)-6H,7H-thieno[2,3-d]pyridazin-6-yl}-N-(pyrimidin-2-yl)acetamide

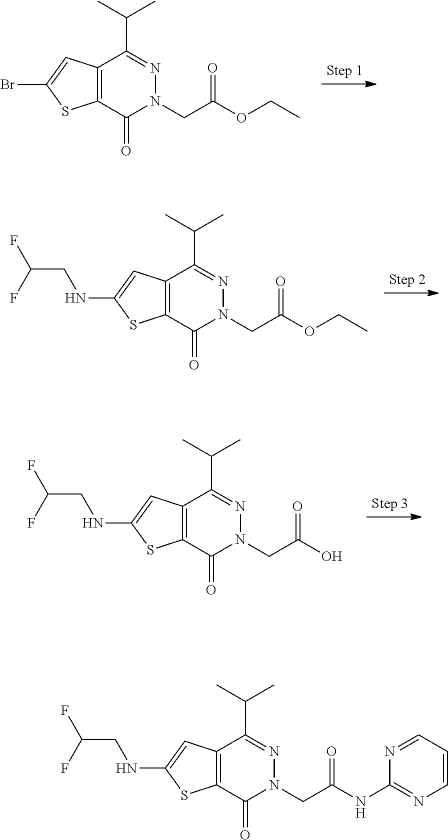 [SMILES: CCOC(=O)Cn1nc(C(C)C)c2cc(Br)sc2c1=O ; CCOC(=O)Cn1nc(C(C)C)c2cc(NCC(F)F)sc2c1=O ; CC(C)c1nn(CC(=O)O)c(=O)c2sc(NCC(F)F)cc12 ; CC(C)c1nn(CC(=O)Nc2ncccn2)c(=O)c2sc(NCC(F)F)cc12]

Step 1. ethyl 2-[2-(2,2-difluoroethylamino)-4-isopropyl-7-oxo-thieno[2,3-d]pyridazin-6-yl]acetate. To a solution of ethyl 2-(2-bromo-4-isopropyl-7-oxo-thieno[2,3-d]pyridazin-6-yl)acetate (Intermediate D4, 300 mg, 0.84 mmol) in dioxane (3 mL) at 25° C. under N₂ was added Cs₂CO₃ (816 mg, 2.51 mmol), Pd(OAc)₂ (18.8 mg, 84 μmol) and (5-diphenylphosphanyl-9,9-dimethyl-xanthen-4-yl)-diphenyl-phosphane (48 mg, 84 μmol). The RM was treated with 2,2-difluoroethanamine (66 μL, 2.51 mmol) and the solution stirred at 105° C. for 2 h. The reaction mixture was concentrated under reduced pressure. The residue was diluted with water (5 mL) and extracted with ethyl acetate (3×2 mL). The combined organic layers were washed with brine (2×2 mL), dried over Na₂SO₄, filtered and concentrated under reduced pressure to give a residue. The residue was purified by prep-TLC (SiO₂, 1:1 EtOAc in petroleum ether) to give the title compound as a yellow solid. Y=43%. ¹H NMR (400 MHz, DMSO-d₆) δ 7.88 (t, J=7 Hz, 1H), 6.39 (s, 1H), 6.36-6.07 (m, 1H), 4.80 (s, 2H), 4.16-4.11 (m, 2H), 4.16-4.10 (m, 2H), 3.71-3.65 (m,H), 3.18-3.16 (m, 1H), 1.25-1.23 (m, 6H), 1.22-1.18 (m, 3H).

Step 2. 2-[2-(2, 2-Difluoroethylamino)-4-isopropyl-7-oxo-thieno[2,3-d]pyridazin-6-yl]acetic acid. To a solution of ethyl 2-[2-(2,2-difluoroethylamino)-4-isopropyl-7-oxo-thieno[2,3-d]pyridazin-6-yl]acetate (120 mg, 0.33 mmol) in THE (2 mL) and H₂O (2 mL) at 0° C. under N₂ was added LiOH·H₂O (42 mg, 1.00 mmol). The solution was stirred at 0° C. for 3 h. The reaction mixture was concentrated under reduced pressure. The resulting residue was diluted with H₂O (3 mL) and washed with ethyl acetate (3×1 mL). The aqueous phase was adjusted to pH˜5 with 2 M HCl and extracted with ethyl acetate (3×1 mL). The combined organic layers were washed with brine (2×1 mL), dried over Na₂SO₄, filtered and concentrated under reduced pressure to give the title compound as a yellow solid. ¹H NMR (400 MHz, DMSO-d₆) δ 7.86 (t, J=7 Hz, 1H), 6.38 (s, 1H), 6.38-6.06 (m, 1H) 4.71 (s, 2H), 3.74-3.65 (m, 2H), 3.17-3.14 (m, 1H), 1.22 (d, J=7 Hz, 6H).

Step 3. 2-{2-[(2,2-Difluoroethyl)amino]-7-oxo-4-(propan-2-yl)-6H, 7H-thieno[2,3-d]pyridazin-6-yl}-N-(pyrimidin-2-yl)acetamide. To a solution of 2-[2-(2,2-difluoroethylamino)-4-isopropyl-7-oxo-thieno[2,3-d]pyridazin-6-yl]acetic acid (75 mg, 226 μmol) and pyrimidin-2-amine (65 mg, 0.68 mmol) in ACN (1 mL) at 25° C. was added COMU (145 mg, 0.34 mmol) and NMM (25 μL, 226 μmol). The RM was stirred at 50° C. for 24 h under N₂. The reaction mixture was directly purified by prep-HPLC (column: Waters Xbridge Prep OBD C18 150×40 mm×10 μm; mobile phase: [water (NH₄HCO₃)−ACN]; B: 20-50%, 8 min) and lyophilised to give the title compound as a white solid. Y=40%. ¹H NMR (400 MHz, DMSO-d₆) δ 10.88 (s, 1H), 8.67 (d, J=5 Hz, 2H), 7.85 (t, J=6 Hz, 1H), 7.20 (t, J=5 Hz, 1H), 6.39 (s, 1H), 6.39-6.07 (m, 1H), 5.09 (s, 2H), 3.74-3.67 (m, 2H), 3.18-3.13 (m, 1H), 1.23 (d, J=7 Hz, 6H). ¹H NMR (400 MHz, DMSO-d₆+D₂O) δ 8.64 (d, J=5 Hz, 2H), 7.19 (t, J=5 Hz, 1H), 6.36 (s, 1H), 6.32-6.03 (m, 1H), 5.06 (s, 2H), 3.72-3.67 (m, 2H), 3.18-3.11 (m, 1H), 1.22-1.20 (d, J=7 Hz, 6H). LCMS (ESI): m/z: [M+H]⁺=409.2.

### Compound 96. 2-{2-[(2-Methoxyethyl)amino]-7-oxo-4-(propan-2-yl)-6H,7H-thieno[2,3-d]pyridazin-6-yl}-N-(pyrimidin-2-yl)acetamide

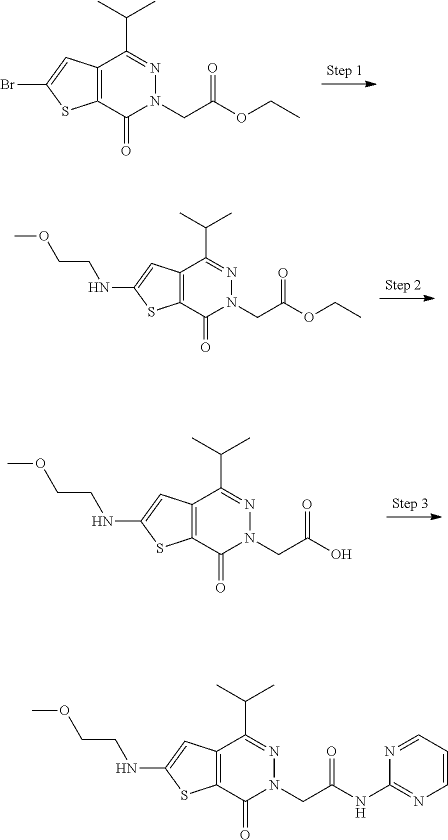

Step 1. Ethyl 2-[4-isopropyl-2-(2-methoxyethylamino)-7-oxo-thieno[2,3-d]pyridazin-6-yl]acetate. To a solution of ethyl 2-(2-bromo-4-isopropyl-7-oxo-thieno[2,3-d]pyridazin-6-yl)acetate (Intermediate D4, 700 mg, 1.95 mmol) in dioxane (7 mL) at 25° C. were added BINAP (121 mg, 195 μmol), 2-methoxyethanamine (508 μL, 5.85 mmol), Cs₂CO₃ (952 mg, 2.92 mmol) and Pd(OAc)₂ (44 mg, 195 μmol). The mixture was stirred at 105° C. for 12 h. The mixture was diluted with H₂O (10 mL) and the resulting mixture extracted with ethyl acetate (3×10 mL). The combined organic layers were washed with brine (10 mL), dried over Na₂SO₄, filtered and concentrated under reduced pressure to give a residue. The residue was purified by column chromatography (25% EtOAc in petrol) to give the title compound as a white solid. Y=20%.

Step 2. 2-[4-Isopropyl-2-(2-methoxyethylamino)-7-oxo-thieno[2,3-d]pyridazin-6-yl]acetic acid. To a solution of ethyl 2-[4-isopropyl-2-(2-methoxyethylamino)-7-oxo-thieno[2,3-d]pyridazin-6-yl]acetate (130 mg, 0.37 mmol) in THE (1 mL) and H₂O (1 mL) at 25° C. under N₂ was added LiOH·H₂O (31 mg, 0.74 mmol). The mixture was stirred at 25° C. for 1 h. The mixture was adjusted to pH˜4 with aqueous 2 M HCl and the resulting mixture was extracted with EtOAc (3×3 mL). The combined organic layers were washed with brine (3 mL), dried over Na₂SO₄, filtered and concentrated under reduced pressure to give the title compound as a white solid. Y=84%. ¹H NMR (400 MHz, DMSO-d₆) δ 12.84 (br. s, 1H), 7.68 (t, J=6 Hz, 1H), 6.16 (s, 1H), 4.70 (s, 2H), 3.53 (t, J=5 Hz, 2H), 3.35 (d, J=5 Hz, 2H), 3.29 (s, 3H), 3.19-3.09 (m, 1H), 1.22 (d, J=7 Hz, 6H).

Step 3. 2-{2-[(2-Methoxyethyl)amino]-7-oxo-4-(propan-2-yl)-6H, 7H-thieno[2,3-d]pyridazin-6-yl}-N-(pyrimidin-2-yl)acetamide. To a solution of 2-[4-isopropyl-2-(2-methoxyethylamino)-7-oxo-thieno[2,3-d]pyridazin-6-yl]acetic acid (100 mg, 0.31 mmol) in ACN (1 mL) at 25° C. was added pyrimidin-2-amine (88 mg, 0.92 mmol), NMM (34 μL, 0.31 mmol) and COMU (263 mg, 0.61 mmol). The mixture was stirred at 50° C. for 12 h. The mixture was diluted with H₂O (3 mL) and extracted with ethyl acetate (3×3 mL). The combined organic layers were washed with brine (3 mL), dried over Na₂SO₄, filtered and concentrated under reduced pressure to give a residue. The residue was purified by prep-HPLC (column: Waters Xbridge Prep OBD C₁₈ 150×40 mm×10 μm; mobile phase: [water (NH₄HCO₃)−ACN]; B: 20-50%, 8 min) and lyophilised to give the title compound as a white solid. Y=31%. ¹H NMR (400 MHz, DMSO-d₆) δ 10.85 (s, 1H), 8.67 (d, J=5 Hz, 2H), 7.66 (t, J=6 Hz, 1H), 7.20 (t, J=5 Hz, 1H), 6.16 (s, 1H), 5.08 (s, 2H), 3.56-3.52 (m, 2H), 3.36-3.33 (m, 2H), 3.29 (s, 3H), 3.20-3.10 (m, 1H), 1.22 (d, J=7 Hz, 6H). ¹H NMR (400 MHz, DMSO-d₆+D₂O) δ 8.66 (d, J=5 Hz, 2H), 7.19 (t, J=5 Hz, 1H), 6.17 (s, 1H), 5.07 (s, 2H), 3.55-3.50 (m, 2H), 3.36-3.33 (m, 2H), 3.29 (s, 3H), 3.19-3.08 (m, 1H), 1.22 (d, J=7 Hz, 6H). LCMS (ESI): m/z: [M+H]⁺=403.2.

### Compound 97. 2-{2-[Ethyl(methyl)amino]-7-oxo-4-(propan-2-yl)-6H,7H-thieno[2,3-d]pyridazin-6-yl}-N-(pyrimidin-2-yl)acetamide

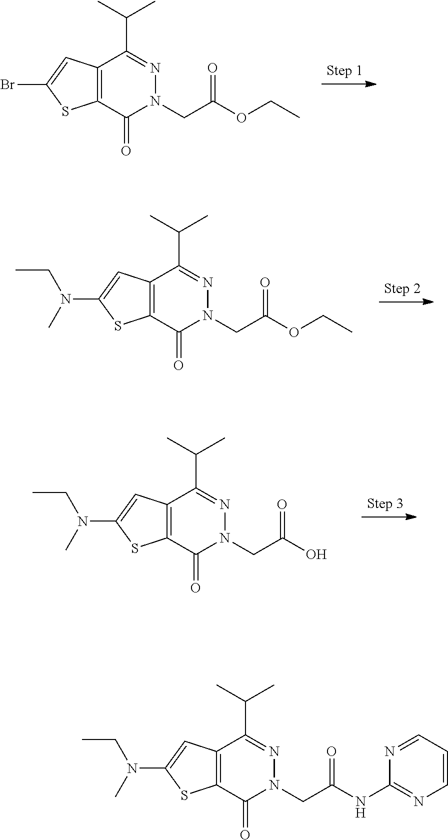

Step 1. Ethyl 2-[2-[ethyl(methyl)amino]-4-isopropyl-7-oxo-thieno[2,3-d]pyridazin-6-yl]acetate. To a solution of ethyl 2-(2-chloro-4-isopropyl-7-oxo-thieno[2,3-d]pyridazin-6-yl)acetate (0.70 g, 2.22 mmol) in dioxane (7 mL) at 25° C. under N₂ was added BINAP (138 mg, 222 μmol), N-methylethanamine (573 μL 6.67 mmol), Cs₂CO₃ (2.17 g, 6.67 mmol) and Pd(OAc)₂ (50 mg, 222 μmol). The mixture was stirred at 105° C. for 12 h. The RM was diluted with H₂O (2 mL) and the resulting mixture was extracted with EtOAc (5×3 mL). The combined organic layers were washed with brine (3 mL), dried over Na₂SO₄, filtered and concentrated under reduced pressure to give a residue. The residue was purified by prep-TLC (SiO₂, 25% EtOAc in petrol) to give the title compound as a yellow solid. Y=53%. ¹H NMR (400 MHz, DMSO-d₆) δ 6.20 (s, 1H), 4.80 (s, 2H), 4.14 (q, J=7 Hz, 2H), 3.47 (q, J=7 Hz, 2H), 3.25-3.17 (m, 1H), 3.04 (s, 3H), 1.24-1.14 (m, 12H).

Step 2. 2-[2-[ethyl(methyl)amino]-4-isopropyl-7-oxo-thieno[2,3-d]pyridazin-6-yl]acetic acid. To a solution of ethyl 2-[2-[ethyl(methyl)amino]-4-isopropyl-7-oxo-thieno[2,3-d]pyridazin-6-yl]acetate (300 mg, 0.89 mmol) in THE (3 mL) and H₂O (1 mL) at 25° C. was added LiOH·H₂O (75 mg, 1.78 mmol). The mixture was stirred at 25° C. for 2 h. The reaction mixture was adjusted to pH ˜4 with 2 M HCl and the resulting mixture extracted with ethyl acetate (3×5 mL). The combined organic layers were washed with brine (5 mL), dried over Na₂SO₄, filtered and concentrated under reduced pressure to give the title compound as a white solid. Y=91%. ¹H NMR (400 MHz, DMSO-d₆) δ 6.19 (s, 1H), 4.71 (s, 2H), 3.47 (q, J=7 Hz, 2H), 3.25-3.15 (m, 1H), 3.04 (s, 3H), 1.23 (d, J=7 Hz, 6H), 1.16 (t, J=7 Hz, 3H).

Step 3. 2-{2-[Ethyl(methyl)amino]-7-oxo-4-(propan-2-yl)-6H, 7H-thieno[2,3-d]pyridazin-6-yl}-N-(pyrimidin-2-yl)acetamide. To a solution of 2-[2-[ethyl(methyl)amino]-4-isopropyl-7-oxo-thieno[2,3-d]pyridazin-6-yl]acetic acid (0.10 g, 323 μmol) in ACN (1 mL) at 25° C. was added COMU (208 mg, 0.48 mmol), pyrimidin-2-amine (92 mg, 0.97 mmol) and NMM (36 μL 0.32 mmol). The mixture was stirred at 50° C. for 12 h. The reaction mixture was concentrated under reduced pressure to remove ACN (1 mL). The residue was diluted with H₂O (2 mL) and the resulting mixture was extracted with DCM (3×2 mL). The combined organic layers were washed with brine (2×1 mL), dried over Na₂SO₄, filtered and concentrated under reduced pressure to give a residue. The residue was purified by prep-HPLC (column: Waters Xbridge Prep OBD C18 150×40 mm×10 μm; mobile phase: [water (NH₄HCO₃) −ACN]; B: 25-55%, 8 min) and lyophilised to give the title compound as a white solid. Y=27%. ¹H NMR (400 MHz, DMSO-d₆) δ 10.87 (s, 1H), 8.67 (d, J=5 Hz, 2H), 7.20 (t, J=5 Hz, 1H), 6.20 (s, 1H), 5.09 (s, 2H), 3.47 (q, J=7 Hz, 2H), 3.25-3.15 (m, 1H), 3.04 (s, 3H), 1.23 (d, J=7 Hz, 6H), 1.16 (t, J=7 Hz, 3H). ¹H NMR (400 MHz, DMSO-d₆+D₂O) δ 10.86 (s, 0.1H), 8.66 (d, J=5 Hz, 2H), 7.19 (t, J=5 Hz, 1H), 6.18 (s, 1H), 5.08 (s, 2H), 3.47 (q, J=7 Hz, 2H), 3.25-3.15 (m, 1H), 3.03 (s, 3H), 1.23 (d, J=7 Hz, 6H), 1.16 (t, J=7 Hz, 3H). LCMS (ESI): m/z: [M+H]⁺=387.2.

### Compound 98. 2-[2-(Ethylamino)-4-methyl-7-oxo-6H,7H-thieno[2,3-d]pyridazin-6-yl]-N-(pyrimidin-2-yl)acetamide

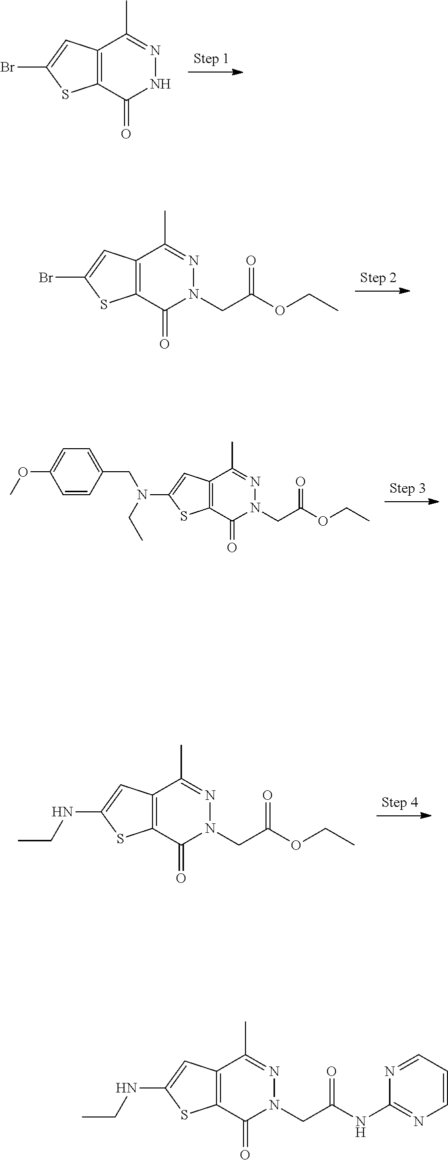

Step 1. Ethyl 2-(2-bromo-4-methyl-7-oxo-thieno [2,3-d]pyridazin-6-yl)acetate. To a solution of 2-bromo-4-methyl-6H-thieno[2,3-d]pyridazin-7-one (for synthesis see Intermediate D2) (400 mg, 1.63 mmol) in DMF (4 mL) at 25° C. under N₂ was added ethyl 2-bromoacetate (271 μL, 2.45 mmol) and K₂CO₃ (451 mg, 3.26 mmol). The RM was stirred at 80° C. for 2 h. The reaction mixture was diluted with H₂O (8 mL) and extracted with EtOAc (3×6 mL). The combined organic layers were washed with brine (2×5 mL), dried over Na₂SO₄, filtered and concentrated under reduced pressure to give a residue. The residue was purified by column chromatography (25-50% EtOAc in petrol) to give the title compound as a white solid. Y=93%. ¹H NMR (400 MHz, DMSO-d₆) δ 7.90 (s, 1H), 4.90 (s, 2H), 4.18-4.13 (m, 2H), 2.49 (s, 3H), 1.22-1.19 (m, 3H).

Step 2. Ethyl 2-[2-[ethyl-[(4-methoxyphenyl)methyl]amino]-4-methyl-7-oxo-thieno[2,3-d]pyridazin-6-yl]acetate. To a solution of ethyl 2-(2-bromo-4-methyl-7-oxo-thieno[2,3-d]pyridazin-6-yl)acetate (190 mg, 0.57 mmol) in dioxane (2 mL) at 25° C. under N₂ was added BINAP (36 mg, 57 μmol), N-[(4-methoxyphenyl)methyl]ethanamine (284 mg, 1.72 mmol), Pd(OAc)₂ (13 mg, 57 μmol) and Cs₂CO₃ (280 mg, 0.86 mmol). The mixture was stirred at 105° C. for 4 h. The reaction mixture was diluted with H₂O (2 mL) and extracted with ethyl acetate (3×2 mL). The combined organic layers were washed with brine (2×2 mL), dried over Na₂SO₄, filtered and concentrated under reduced pressure to give a residue. The residue was purified by column chromatography (17-25% EtOAc in petrol) to give the title compound as a white solid. Y=42%.

Step 3. Ethyl 2-[2-(ethylamino)-4-methyl-7-oxo-thieno[2,3-d]pyridazin-6-yl]acetate. A mixture of ethyl 2-[2-[ethyl-[(4-methoxyphenyl)methyl]amino]-4-methyl-7-oxo-thieno[2,3-d]pyridazin-6-yl]acetate (500 mg, 1.20 mmol) and TFA (5 mL) was stirred at 70° C. under N₂ for 1 h. The residue was concentrated under reduced pressure and the resulting residue purified by column chromatography (25-50% EtOAc in petrol) to give the title compound as a white solid. Y=42%.

Step 4. Pyrimidin-2-yl 2-[2-(ethylamino)-4-methyl-7-oxo-6H, 7H-thieno[2,3-d]pyridazin-6-yl]acetate. To a solution of 2-[2-(ethylamino)-4-methyl-7-oxo-thieno[2,3-d]pyridazin-6-yl]acetic acid (110 mg, 0.41 mmol) in ACN (1.5 mL) at 25° C. under N₂ was added COMU (264 mg, 0.62 mmol), pyrimidin-2-amine (78 mg, 0.82 mmol) and NMM (45 μL 0.41 mmol). The mixture was stirred at 50° C. for 4 h. The mixture was diluted with H₂O (2 mL) and the resulting mixture extracted with ethyl acetate (3×2 mL). The combined organic layers were washed with brine (2×2 mL), dried over Na₂SO₄, filtered and concentrated under reduced pressure to give a residue. The residue was purified by prep-HPLC (column: Waters Xbridge Prep OBD C18 150×40 mm×10 μm; mobile phase: [water (NH₄HCO₃)−ACN]; B: 10-40%, 8 min) and lyophilised to give the title compound as a white solid. Y=28%. ¹H NMR (400 MHz, DMSO-d₆) δ 10.87 (s, 1H), 8.67 (d, J=5 Hz, 2H), 7.59 (t, J=5 Hz, 1H), 7.20 (t, J=5 Hz, 1H), 6.03 (s, 1H), 5.07 (s, 2H), 3.24-3.15 (m, 2H), 2.35 (s, 3H), 1.21 (t, J=7 Hz, 3H). ¹H NMR (400 MHz, DMSO-d₆+D₂O) δ 8.65 (d, J=5 Hz, 2H), 7.19 (t, J=5 Hz, 1H), 6.04 (s, 1H), 5.05 (s, 2H), 3.26-3.08 (m, 2H), 2.35 (s, 3H), 1.20 (t, J=7 Hz, 3H). LC-MS (ESI): m/z: [M+H]⁺=345.2.

### Compound 99. 2-[7-Oxo-4-(propan-2-yl)-2-propyl-6H,7H-thieno[2,3-d]pyridazin-6-yl]-N-(pyrimidin-2-yl)acetamide

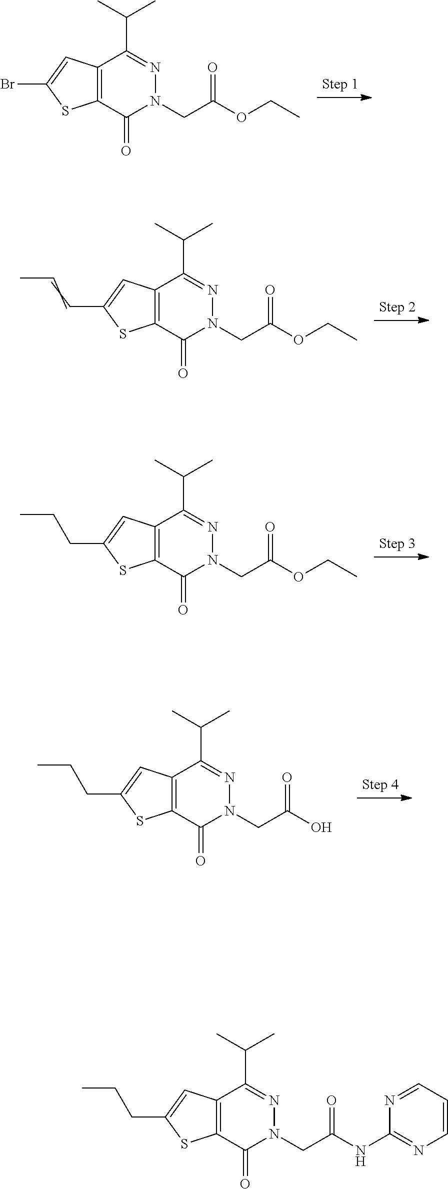

Step 1. Ethyl 2-[7-oxo-2-(prop-1-en-1-yl)-4-(propan-2-yl)-6H,7H-thieno[2,3-d]pyridazin-6-yl]acetate. To a mixture of ethyl 2-(2-bromo-4-isopropyl-7-oxo-thieno[2,3-d]pyridazin-6-yl)acetate (Intermediate D4, 400 mg, 1.11 mmol) and 4,4,5,5-tetramethyl-2-prop-1-enyl-1,3,2-dioxaborolane (281 mg, 1.67 mmol) in dioxane (3 mL) and H₂O (1 mL) at 25° C. under N₂ was added Na₂CO₃ (236 mg, 2.23 mmol) and Pd(dppf)Cl₂ (163 mg, 0.22 mmol). The mixture was stirred at 80° C. for 8 h. The reaction mixture was diluted with H₂O (3 mL) and the resulting mixture extracted with ethyl acetate (3×5 mL). The combined organic phase was washed with brine (2×5 mL), dried with anhydrous Na₂SO₄, filtered and concentrated under vacuum. The residue was purified by column chromatography (20% EtOAc in petrol) to give the title compound as a yellow oil. Y=98%. ¹H NMR (400 MHz, DMSO-d₆) δ 7.55 (s, 1H), 6.85-6.75 (m, 1H), 6.53-6.42 (m, 1H), 4.89 (s, 2H), 4.20-4.11 (m, 2H), 3.32-3.26 (m, 1H), 1.90 (d, J=7 Hz, 3H), 1.26 (d, J=7 Hz, 6H), 1.23-1.18 (m, 3H).

Step 2. Ethyl 2-(4-isopropyl-7-oxo-2-propyl-thieno [2,3-d]pyridazin-6-yl) acetate. To a solution of ethyl 2-[7-oxo-2-(prop-1-en-1-yl)-4-(propan-2-yl)-6H,7H-thieno[2,3-d]pyridazin-6-yl]acetate (0.35 g, 1.09 mmol) in EtOH (10 mL) under N₂ was added 10% Pd/C (50 wt % in water, 50 mg). The suspension was degassed under vacuum and purged with H₂ several times. The mixture was stirred under H₂ (15 psi) at 25° C. for 2 h. The reaction was filtered and the filtrate was concentrated under vacuum to give the title compound as a yellow solid. Y=91%. ¹H NMR (400 MHz, DMSO-d₆) δ 7.46 (s, 1H), 4.89 (s, 2H), 4.15 (q, J=7 Hz, 2H), 3.48-3.40 (m, 1H), 2.96 (t, J=7 Hz, 2H), 1.73 (t, J=7 Hz, 2H), 1.26 (d, J=7 Hz, 6H), 1.20 (t, J=7 Hz, 3H), 0.96 (t, J=7 Hz, 3H).

Step 3. 2-(4-Isopropyl-7-oxo-2-propyl-thieno [2,3-d]pyridazin-6-yl) acetic acid. To a mixture of ethyl 2-(4-isopropyl-7-oxo-2-propyl-thieno[2,3-d]pyridazin-6-yl) acetate (320 mg, 0.99 mmol) in THE (4 mL) and H₂O (1 mL) was added LiOH·H₂O (83 mg, 1.98 mmol). The mixture was stirred at 25° C. for 2 h. The reaction was adjusted to pH ˜4 with 2 M HCl and the resulting mixture extracted with ethyl acetate (3×3 mL). The combined organic phase was washed with brine (2×3 mL), dried with anhydrous Na₂SO₄, filtered and concentrated under vacuum to give the title compound as a yellow solid. Y=89%. ¹H NMR (400 MHz, DMSO-d₆) δ 13.00 (br. s, 1H), 7.46 (s, 1H), 4.80 (s, 2H), 3.35-3.28 (m, 1H), 2.96 (t, J=7 Hz, 2H), 1.73 (t, J=7 Hz, 2H), 1.26 (d, J=7 Hz, 6H), 0.96 (t, J=7 Hz, 3H).

Step 4. 2-[7-Oxo-4-(propan-2-yl)-2-propyl-6H,7H-thieno[2,3-d]pyridazin-6-yl]-N-(pyrimidin-2-yl)acetamide. To a mixture of 2-(4-isopropyl-7-oxo-2-propyl-thieno[2,3-d]pyridazin-6-yl)acetic acid (100 mg, 0.34 mmol) and pyrimidin-2-amine (39 mg, 0.41 mmol) in ACN (2 mL) at 25° C. was added COMU (218 mg, 0.51 mmol) and NMM (56 μL, 0.51 mmol). The mixture was stirred at 50° C. for 8 h. The reaction was concentrated under vacuum. The residue was purified by prep-HPLC (column: Waters Xbridge Prep OBD C₁₈ 150×40 mm×10 μm; mobile phase: [water (NH₄HCO₃)−ACN]; B: 30-60%, 8 min) and lyophilised to give the title compound as a white solid. Y=17%. ¹H NMR (400 MHz, DMSO-d₆) δ 10.94 (s, 1H), 8.68 (d, J=5 Hz, 2H), 7.47 (s, 1H), 7.21 (t, J=5 Hz, 1H), 5.17 (s, 2H), 3.35-3.25 (m, 1H), 2.97 (t, J=7 Hz, 2H), 1.79-1.70 (m, 2H), 1.26 (d, J=7 Hz, 6H), 0.95 (t, J=7 Hz, 3H). ¹H NMR (400 MHz, DMSO-d₆+D₂O) δ 8.66 (d, J=5 Hz, 2H), 7.44 (s, 1H), 7.20 (t, J=5 Hz, 1H), 5.15 (s, 2H), 3.35-3.30 (m, 1H), 2.96 (t, J=7 Hz, 2H), 1.79-1.70 (m, 2H), 1.25 (d, J=7 Hz, 6H), 0.95 (t, J=7 Hz, 3H). LCMS (ESI): m/z: [M+H]⁺=372.2.

### Compound 100. 2-[7-Oxo-4-(propan-2-yl)-2-[(2,2,2-trifluoroethyl)amino]-6H,7H-thieno[2,3-d]pyridazin-6-yl]-N-(pyrimidin-2-yl)acetamide

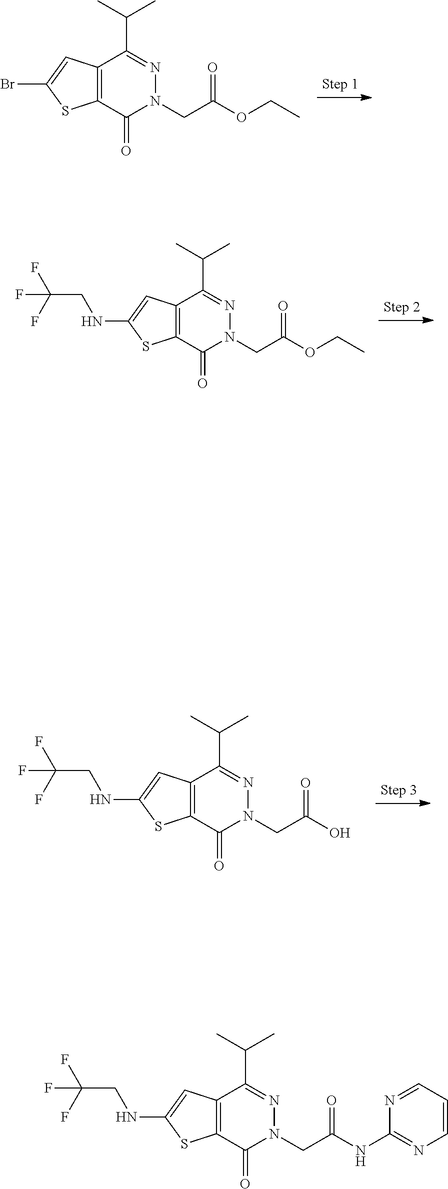 [SMILES: CCOC(=O)Cn1nc(C(C)C)c2cc(Br)sc2c1=O ; CCOC(=O)Cn1nc(C(C)C)c2cc(NCC(F)(F)F)sc2c1=O ; CC(C)c1nn(CC(=O)O)c(=O)c2sc(NCC(F)(F)F)cc12 ; CC(C)c1nn(CC(=O)Nc2ncccn2)c(=O)c2sc(NCC(F)(F)F)cc12]

Prepared in an analogous way to Compound 95. ¹H NMR (400 MHz, DMSO-d₆) δ 10.88 (s, 1H), 8.67 (d, J=5 Hz, 2H), 8.05 (t, J=7 Hz, 1H), 7.20 (t, J=5 Hz, 1H), 6.52 (s, 1H), 5.09 (s, 2H), 4.22-4.14 (m, 2H), 3.19-3.12 (m, 1H), 1.23 (d, J=7 Hz, 6H). ¹H NMR (400 MHz, DMSO-d₆+D₂O) δ 8.65 (d, J=5 Hz, 2H), 7.19 (t, J=5 Hz, 1H), 6.50 (s, 1H), 5.07 (s, 2H), 4.18-4.11 (m, 2H), 3.20-3.09 (m, 1H), 1.22 (d, J=7 Hz, 6H). LCMS (ESI): m/z: [M+H]⁺=427.1.

### Compound 101. 2-[2-Amino-7-oxo-4-(propan-2-yl)-6H,7H-thieno[2,3-d]pyridazin-6-yl]-N-(pyrimidin-2-yl)acetamide

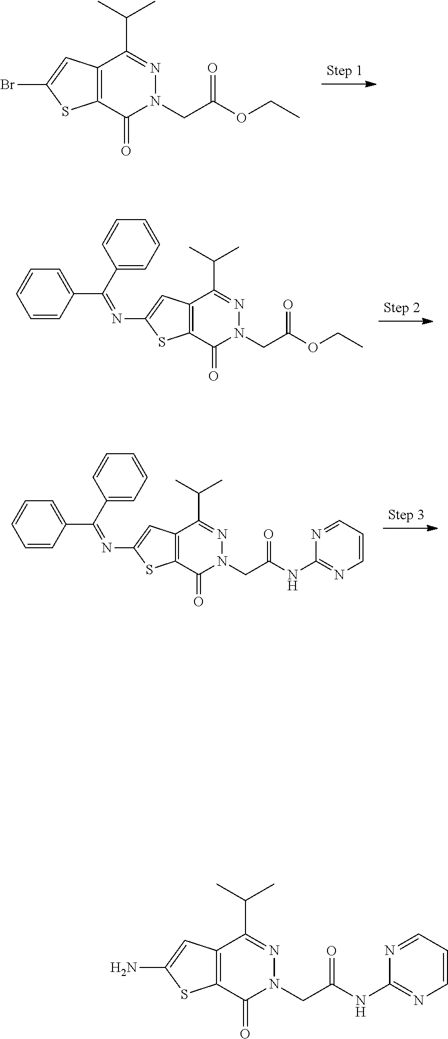 [SMILES: CCOC(=O)Cn1nc(C(C)C)c2cc(Br)sc2c1=O ; CCOC(=O)Cn1nc(C(C)C)c2cc(N=C(c3ccccc3)c3ccccc3)sc2c1=O ; CC(C)c1nn(CC(=O)Nc2ncccn2)c(=O)c2sc(N=C(c3ccccc3)c3ccccc3)cc12 ; CC(C)c1nn(CC(=O)Nc2ncccn2)c(=O)c2sc(N)cc12]

Step 1. Ethyl 2-[2-(benzhydrylideneamino)-4-isopropyl-7-oxo-thieno[2,3-d]pyridazin-6-yl]acetate. To a solution of ethyl 2-(2-bromo-4-isopropyl-7-oxo-thieno[2,3-d]pyridazin-6-yl)acetate (Intermediate D4, 0.70 g, 1.95 mmol) in dioxane (7 mL) at 25° C. was added Pd(OAc)₂ (44 mg, 0.19 mmol), diphenylmethanimine (392 μL, 2.34 mmol), (5-diphenylphosphanyl-9,9-dimethyl-xanthen-4-yl)-diphenyl-phosphane (113 mg, 0.19 mmol) and Cs₂CO₃ (1.27 g, 3.90 mmol). The mixture was stirred at 105° C. for 12 h under N₂. The reaction mixture was diluted with H₂O (10 mL) and extracted with DCM (3×10 mL). The combined organic layers were washed with brine (3×10 mL), dried over Na₂SO₄, filtered and concentrated under reduced pressure to give a residue. The residue was purified by column chromatography (10-25% EtOAc in petrol) to give the title compound as a yellow solid. Y=89%. ¹H NMR (400 MHz, DMSO-d₆) δ 7.80-7.70 (m, 2H), 7.65-7.55 (m, 4H), 7.54-7.50 (m 2H), 7.45-7.32 (m, 3H), 4.82 (s, 2H), 4.12 (q, J=7 Hz, 2H), 3.30-3.22 (m, 1H), 1.25-1.15 (m, 9H).

Step 2. 2-[2-(Benzhydrylideneamino)-4-isopropyl-7-oxo-thieno[2,3-d]pyridazin-6-yl]-N-pyrimidin-2-yl-acetamide. To a solution of ethyl 2-[2-(benzhydrylideneamino)-4-isopropyl-7-oxo-thieno[2,3-d]pyridazin-6-yl]acetate (0.50 g, 1.09 mmol) and pyrimidin-2-amine (207 mg, 2.18 mmol) in THE (5 mL) at 0° C. was added LiHMDS (1 M in THF, 2.39 mL, 2.39 mmol). The mixture was stirred at 25° C. for 2 h. The reaction mixture was quenched by addition of H₂O (5 mL) at 0° C., and then the resulting mixture was extracted with EtOAc (3×5 mL). The combined organic layers were washed with brine (2×5 mL), dried over Na₂SO₄, filtered and concentrated under reduced pressure to give a residue. The crude product was purified by prep-HPLC (column: Waters Xbridge Prep OBD C18 150×40 mm×10 μm; mobile phase: [water (NH₄ HCO₃)−ACN]; B: 45-75%, 8 min) and lyophilised to give the title compound as a yellow solid. Y=15%. ¹H NMR (400 MHz, DMSO-d₆) δ 10.92 (s, 1H), 8.67 (d, J=5 Hz, 2H), 7.81-7.73 (m, 2H), 7.64-7.57 (m, 4H), 7.55-7.50 (m, 2H), 7.43-7.36 (m, 3H), 7.20 (t, J=5 Hz, 1H), 5.11 (s, 2H), 3.35-3.25 (m, 1H), 1.22 (d, J=7 Hz, 6H). ¹H NMR (400 MHz, DMSO-d₆+D₂O) δ 8.63 (d, J=5 Hz, 2H), 7.77-7.67 (m, 2H), 7.62-7.55 (m, 4H), 7.53-7.47 (m, 2H), 7.37-7.30 (m, 3H), 7.18 (t, J=5 Hz, 1H), 5.06 (s, 2H), 3.32-3.12 (m, 1H), 1.19 (d, J=7 Hz, 6H). LCMS (ESI): m/z: [M+H]⁺=509.2.

Step 3. 2-[2-Amino-7-oxo-4-(propan-2-yl)-6H, 7H-thieno[2,3-d]pyridazin-6-yl]-N-(pyrimidin-2-yl)acetamide. To a solution of 2-[2-(benzhydrylideneamino)-4-isopropyl-7-oxo-thieno[2,3-d]pyridazin-6-yl]-N-pyrimidin-2-yl-acetamide (0.20 g, 0.39 mmol) in MeOH (2 mL) at 25° C. was added hydroxylamine hydrochloride (93 mg, 1.34 mmol) and sodium acetate (97 mg, 1.18 mmol). The mixture was stirred at 25° C. for 2 h. The reaction mixture was concentrated under reduced pressure to remove MeOH (2 mL). The residue was diluted with H₂O (1 mL) and the resulting mixture was extracted with DCM (3×1 mL). The combined organic layers were washed with brine (1 mL), dried over Na₂SO₄, filtered and concentrated under reduced pressure to give a residue. The residue was purified by prep-HPLC (column: Waters Xbridge Prep OBD C18 150×40 mm×10 μm; mobile phase: [water (NH₄HCO₃) −ACN]; gradient: 20-50% B over 8 min) and lyophilised to give the title compound as a white solid. Y=25%. ¹H NMR (400 MHz, DMSO-d₆) δ 10.87 (s, 1H), 8.67 (d, J=5 Hz, 2H), 7.20 (t, J=5 Hz, 1H), 6.99 (d, J=5 Hz, 1H), 6.10 (s, 1H), 5.07 (s, 2H), 3.15-3.05 (m, 1H), 1.21 (d, J=7 Hz, 6H). ¹H NMR (400 MHz, DMSO-d₆+D₂O) δ 8.64 (d, J=5 Hz, 2H), 7.18 (t, J=5 Hz, 1H), 6.14 (s, 1H), 5.04 (s, 2H), 3.20-2.95 (m, 1H), 1.20 (d, J=7 Hz, 6H). LCMS (ESI): m/z: [M+H]⁺=345.2.

### Compound 102. 2-[2-(Ethylamino)-4-oxo-7-(propan-2-yl)-4H,5H-furo[2,3-d]pyridazin-5-yl]-N-(pyrimidin-2-yl)acetamide

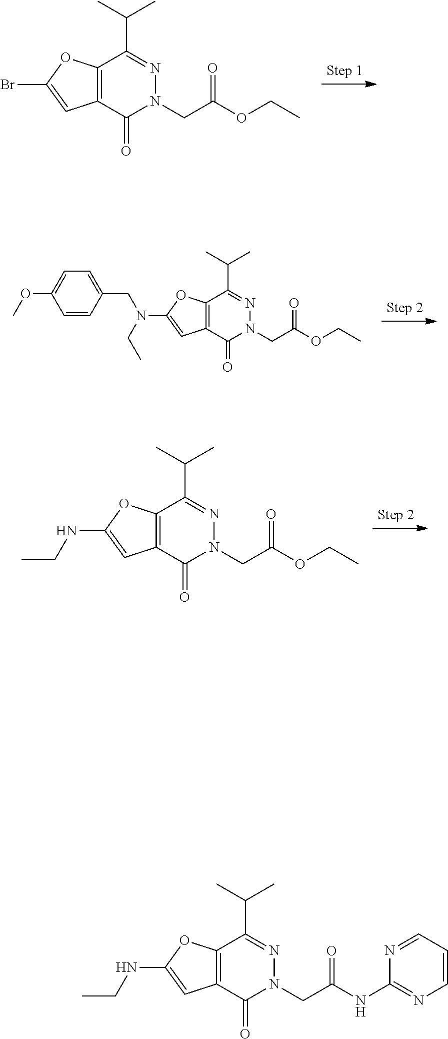

Step 1. Ethyl 2-[2-[ethyl-[(4-methoxyphenyl)methyl]amino]-7-isopropyl-4-oxo-furo[2,3-d]pyridazin-5-yl]acetate. To a solution of ethyl 2-(2-bromo-7-isopropyl-4-oxo-furo[2,3-d]pyridazin-5-yl)acetate (Intermediate D5, 670 mg, 1.95 mmol) in dioxane (10 mL) at 25° C. were added N-[(4-methoxyphenyl)methyl]ethanamine (0.97 g, 5.86 mmol), BINAP (122 mg, 0.20 mmol), Pd(OAc)₂ (44 mg, 0.20 mmol) and Cs₂CO₃ (0.95 g, 2.93 mmol). The mixture was stirred at 105° C. for 2 h. The reaction mixture was diluted with H₂O (10 mL) and extracted with EtOAc (3×10 mL). The combined organic layers were washed with brine (10 mL), dried over Na₂SO₄, filtered and concentrated under reduced pressure to give a residue. The residue was purified by column chromatography (50% EtOAc in petrol) to give the title compound as a white solid. Y=36%. ¹H NMR (400 MHz, DMSO-d₆) δ 7.26 (d, J=9 Hz, 2H) 6.91 (d, J=9 Hz, 2H) 5.64 (s, 1H) 4.80 (s, 2H) 4.52 (s, 2H) 4.13 (q, J=7 Hz, 2H) 3.73 (s, 3H) 3.43 (q, J=7 Hz, 2H) 3.20-3.05 (m, 1H) 1.26 (d, J=7 Hz, 6H) 1.19 (t, J=7 Hz, 3H) 1.14 (t, J=7 Hz, 3H).

Step 2. Ethyl 2-[2-(ethylamino)-7-isopropyl-4-oxo-furo [2,3-d]pyridazin-5-yl]acetate. To a solution of ethyl 2-[2-[ethyl-[(4-methoxyphenyl)methyl]amino]-7-isopropyl-4-oxo-furo[2,3-d]pyridazin-5-yl]acetate (500 mg, 1.17 mmol) in DCM (3 mL) at 25° C. was added TFA (87 μL, 1.17 mmol). The mixture was stirred at 25° C. for 2 h. The reaction mixture was filtered and the filtrate concentrated under reduced pressure to give a residue. The residue was purified by prep-TLC (SiO₂, 1:1 EtOAc/petrol) to give the title compound as a white solid. Y=28%.

Step 3. 2-[2-(Ethylamino)-4-oxo-7-(propan-2-yl)-4H,5H-furo[2,3-d]pyridazin-5-yl]-N-(pyrimidin-2-yl)acetamide. To a solution of ethyl 2-[2-(ethylamino)-7-isopropyl-4-oxo-furo[2,3-d]pyridazin-5-yl]acetate (80 mg, 0.26 mmol) and pyrimidin-2-amine (37 mg, 0.39 mmol) in THE (1 mL) at 0° C. was added LiHMDS (1 M in THF, 0.57 mL, 0.57 mmol). The RM was stirred at 25° C. for 2 h. The reaction mixture was diluted with H₂O (2 mL) and the resulting mixture was extracted with EtOAc (3×2 mL). The combined organic layers were washed with brine (2 mL), dried over Na₂SO₄, filtered and concentrated under reduced pressure to give a residue. The residue was purified by prep-HPLC (column: Waters Xbridge Prep OBD C₁₈ 150×40 mm×10 μm; mobile phase: [water (NH₄HCO₃)−ACN]; B: 20-50%, 8 min) and lyophilised to give the title compound as a white solid. Y=31%. ¹H NMR (400 MHz, DMSO-d₆) δ 10.86 (s, 1H) 8.67 (d, J=5 Hz, 2H) 7.43 (t, J=6 Hz, 1H) 7.20 (t, J=5 Hz, 1H) 5.45 (s, 1H) 5.09 (s, 2H) 3.20-3.09 (m, 3H) 1.28 (d, J=7 Hz, 6H) 1.18 (t, J=7 Hz, 3H). ¹H NMR (400 MHz, DMSO-d₆+D₂O) δ 8.65 (d, J=5 Hz, 2H) 7.19 (t, J=5 Hz, 1H) 5.45 (s, 1H) 5.07 (s, 2H) 3.22-3.07 (m, 3H) 1.27 (d, J=7 Hz, 6H) 1.16 (t, J=7 Hz, 3H). LCMS (ESI): m/z [M+H]⁺=357.2.

### Compound 103. 2-{2-Cyclopropyl-7-methyl-4-oxo-4H,5H-furo[2,3-d]pyridazin-5-yl}-N-{[1,2,4]triazolo[4,3-a]pyridin-6-yl}acetamide

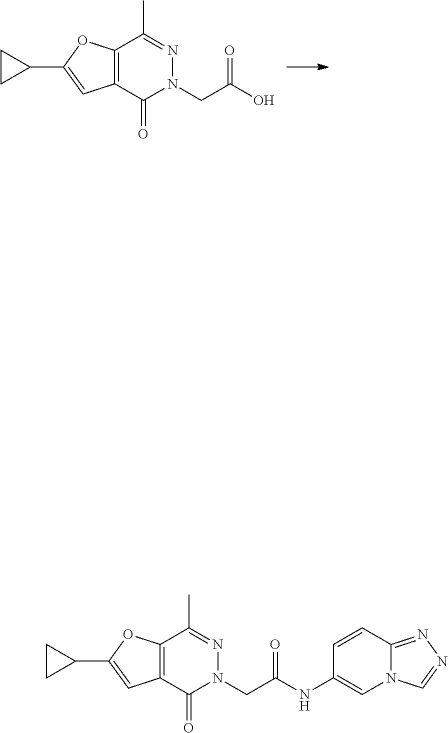 [SMILES: Cc1nn(CC(=O)O)c(=O)c2cc(C3CC3)oc12 ; Cc1nn(CC(=O)Nc2ccc3nncn3c2)c(=O)c2cc(C3CC3)oc12]

Prepared in an analogous way to Compound 82 using Intermediate D1 and [1,2,4]triazolo[4,3-a]pyridin-6-amine to give the title compound as a white solid. Y=41%. ¹H NMR (400 MHz, DMSO-d₆) δ 10.70 (s, 1H), 9.38 (d, J=2 Hz, 1H), 8.43 (s, 1H), 7.86 (d, J=10 Hz, 1H), 7.68-7.56 (m, 1H), 6.81 (s, 1H), 4.99 (s, 2H), 2.45 (s, 3H), 2.23-2.14 (m, 1H), 1.11-1.04 (m, 2H), 1.00-0.94 (m, 2H). ¹H NMR (400 MHz, DMSO-d₆+D₂O) δ 9.38-9.32 (m, 1H), 8.42 (s, 1H), 7.83 (d, J=10 Hz, 1H), 7.64-7.58 (m, 1H), 6.75 (s, 1H), 4.97 (s, 2H), 2.43 (s, 3H), 2.22-2.13 (m, 1H), 1.10-1.03 (m, 2H), 0.97-0.90 (m, 2H). LC-MS (ESI): m/z: [M+H]⁺=365.0.

### Compound 104. 2-[2-Cyclopropyl-4-oxo-7-(propan-2-yl)-4H,5H-furo[2,3-d]pyridazin-5-yl]-N-{[1,2,4]triazolo[4,3-a]pyridin-6-yl}acetamide

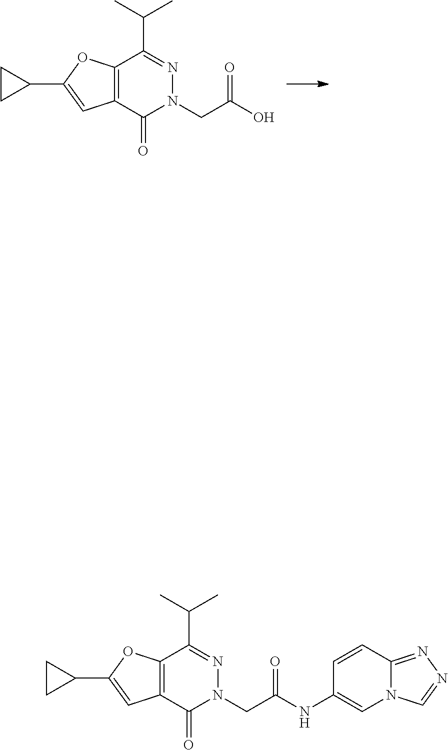 [SMILES: CC(C)c1nn(CC(=O)O)c(=O)c2cc(C3CC3)oc12 ; CC(C)c1nn(CC(=O)Nc2ccc3nncn3c2)c(=O)c2cc(C3CC3)oc12]

To a solution of 2-(2-cyclopropyl-7-isopropyl-4-oxo-furo[2,3-d]pyridazin-5-yl) acetic acid (Intermediate D6, 100 mg, 0.36 mmol) in DMF (1 mL) at 25° C. was added HATU (275 mg, 0.72 mmol). The mixture was stirred at 25° C. for 10 min, then treated with [1,2,4]triazolo[4,3-a]pyridin-6-amine (97 mg, 0.72 mmol) and DIPEA (126 μL, 0.72 mmol) at 25° C. The resulting mixture was stirred at 25° C. for 2 h. The reaction mixture was diluted with H₂O (1 mL) and the resulting mixture was extracted with ethyl acetate (3×1 mL). The combined organic layers were washed with brine (1 mL), dried over Na₂SO₄, filtered and concentrated under reduced pressure to give a residue. The residue was purified by prep-HPLC (column: Waters Xbridge Prep OBD C₁₈ 150×40 mm×10 μm; mobile phase: [water (NH₄HCO₃)−ACN]; B: 35-65%, 8 min) and lyophilised to give the title compound as a white solid. Y=85%. ¹H NMR (400 MHz, DMSO-d₆) δ 10.69 (s, 1H), 9.38 (d, J=2 Hz, 1H), 8.43 (s, 1H), 7.86 (d, J=10 Hz, 1H), 7.62 (dd, J=10, 2 Hz, 1H), 6.80 (s, 1H), 4.98 (s, 2H), 3.27-3.19 (m, 1H), 2.26-2.16 (m, 1H), 1.31 (d, J=7 Hz, 6H), 1.11-1.06 (m, 2H), 0.99-0.94 (m, 2H). ¹H NMR (400 MHz, DMSO-d₆+D₂O) δ 9.32 (d, J=2 Hz, 1H), 8.40 (s, 1H), 7.81 (d, J=10 Hz, 1H), 7.62 (dd, J=10, 2 Hz, 1H), 6.80 (s, 1H), 4.96 (s, 2H), 3.26-3.14 (m, 1H), 2.25-2.09 (m, 1H), 1.26 (d, J=7 Hz, 6H), 1.10-1.06 (m, 2H), 0.95-0.90 (m, 2H). LCMS (ESI): m/z: [M+H]⁺=393.2.

### Compound 105. 2-[2-Cyclopropyl-4-oxo-7-(propan-2-yl)-4H,5H-furo[2,3-d]pyridazin-5-yl]-N-{[1,2,4]triazolo[4,3-b]pyridazin-6-yl}acetamide

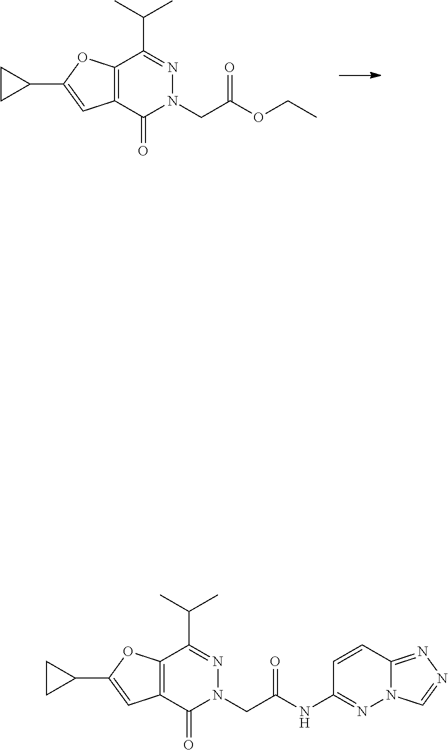 [SMILES: CCOC(=O)Cn1nc(C(C)C)c2oc(C3CC3)cc2c1=O ; CC(C)c1nn(CC(=O)Nc2ccc3nncn3n2)c(=O)c2cc(C3CC3)oc12]

To a solution of ethyl 2-(2-cyclopropyl-7-isopropyl-4-oxo-furo[2,3-d]pyridazin-5-yl)acetate (for synthesis see Intermediate D6)(85 mg, 0.28 mmol) and [1,2,4]triazolo[4,3-b]pyridazin-6-amine (38 mg, 0.28 mmol) in THE (2 mL) at 0° C. under N₂ was added LiHMDS (1 M in THF, 0.61 mL, 0.61 mmol). The mixture was stirred at 25° C. for 2 h. The reaction mixture was cooled to 0° C. and quenched by addition of H₂O (1 mL). The resulting mixture was extracted with ethyl acetate (3×1 mL). The combined organic layers were washed with brine (1 mL), dried over Na₂SO₄ filtered and concentrated under reduced pressure to give a residue. The residue was purified by prep-HPLC (column: Waters Xbridge Prep OBD C₁₈ 150×40 mm×10 μm; mobile phase: [water (NH₄HCO₃)−ACN]; B: 30-60%, 8 min) and lyophilised to give the title compound as a white solid. Y=18%. ¹H NMR (400 MHz, DMSO-d₆) δ 11.40 (s, 1H), 9.51 (s, 1H), 8.33 (d, J=10 Hz, 1H), 7.90 (d, J=10 Hz, 1H), 6.80 (s, 1H), 5.03 (s, 2H), 3.27-3.20 (m, 1H), 2.26-2.13 (m, 1H), 1.30 (d, J=7 Hz, 6H), 1.11-1.06 (m, 2H), 0.99-0.94 (m, 2H). ¹H NMR (400 MHz, DMSO-d₆+D₂O) δ 9.47 (s, 1H), 8.30 (d, J=10 Hz, 1H), 7.89 (d, J=10 Hz, 1H), 6.77 (s, 1H), 5.02 (s, 2H), 3.27-3.20 (m, 1H), 2.26-2.14 (m, 1H), 1.29 (d, J=7 Hz, 6H), 1.11-1.06 (m, 2H), 0.98-0.91 (m, 2H). LCMS (ESI): m/z: [M+H]⁺=394.0.

### Compound 106. 2-{2-Cyclopropyl-7-methyl-4-oxo-4H,5H-furo[2,3-d]pyridazin-5-yl}-N-{[1,2,4]triazolo[4,3-b]pyridazin-6-yl}acetamide

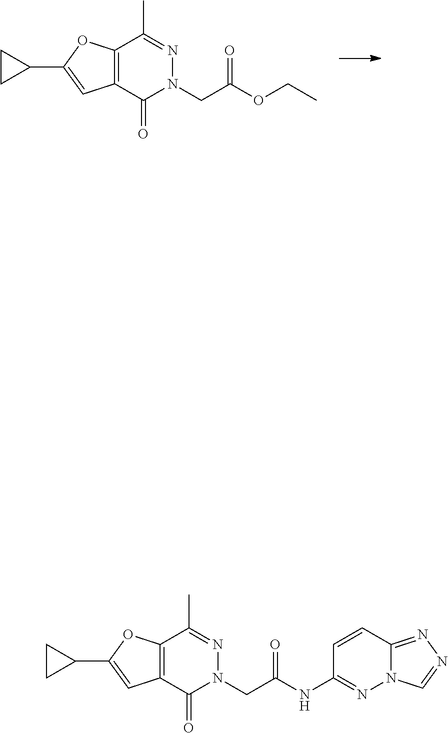 [SMILES: CCOC(=O)Cn1nc(C)c2oc(C3CC3)cc2c1=O ; Cc1nn(CC(=O)Nc2ccc3nncn3n2)c(=O)c2cc(C3CC3)oc12]

To a solution of ethyl 2-(2-cyclopropyl-7-methyl-4-oxo-furo[2,3-d]pyridazin-5-yl)acetate (for synthesis see Intermediate D1)(60 mg, 0.22 mmol) and [1,2,4]triazolo[4,3-b]pyridazin-6-amine (44 mg, 0.33 mmol) in THE (0.6 mL) at 0° C. was added LiHMDS (1 M in THF, 0.48 mL, 0.48 mmol). The mixture was stirred at 25° C. for 1 h. The reaction mixture was quenched by addition of saturated aqueous NH₄Cl (1 mL) and extracted with ethyl acetate (3×1 mL). The combined organic layers were washed with brine (1 mL), dried over Na₂SO₄, filtered and concentrated under reduced pressure to give a residue. The residue was purified by prep-HPLC (column: Waters Xbridge Prep OBD C18 150×40 mm×10 μm; mobile phase: [water (NH₄HCO₃)−ACN]; B: 15-45%, 8 min) and lyophilised to give the title compound as a white solid. Y=37%. ¹H NMR (400 MHz, DMSO-d₆) δ 11.41 (s, 1H), 9.52 (s, 1H), 8.32 (d, J=10 Hz, 1H), 7.91 (d, J=10 Hz, 1H), 6.81 (s, 1H), 5.05 (s, 2H), 2.44 (s, 3H), 2.24-2.13 (m, 1H), 1.10-1.03 (m, 2H), 1.00-0.93 (m, 2H). ¹H NMR (400 MHz, DMSO-d₆+D₂O) δ 9.49 (s, 1H), 8.32 (d, J=10 Hz, 1H), 7.90 (d, J=10 Hz, 1H), 6.78 (s, 1H), 5.04 (s, 2H), 2.43 (s, 3H), 2.23-2.13 (m, 1H), 1.11-1.03 (m, 2H), 0.99-0.91 (m, 2H). LC-MS (ESI): m/z: [M+H]⁺=366.0.

### Compound 107. 2-[2-Ethoxy-7-oxo-4-(propan-2-yl)-6H,7H-thieno[2,3-d]pyridazin-6-yl]-N-(pyrimidin-2-yl)acetamide

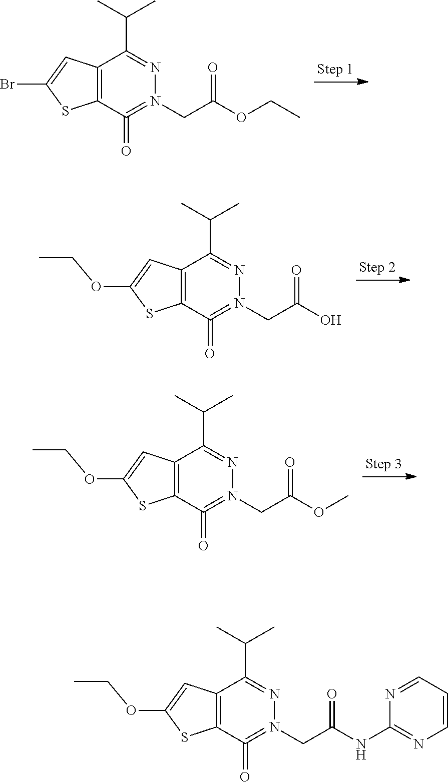

Step 1. 2-(2-Ethoxy-4-isopropyl-7-oxo-thieno [2,3-d]pyridazin-6-yl)acetic acid. To a solution of ethyl 2-(2-bromo-4-isopropyl-7-oxo-thieno[2,3-d]pyridazin-6-yl)acetate (Intermediate D4, 1.0 g, 2.78 mmol) in EtOH (10 mL) at 25° C. were added sodium ethoxide (379 mg, 5.57 mmol) and CuO (111 mg, 1.39 mmol). The mixture was stirred at 80° C. for 12 h. The reaction mixture was quenched with H₂O (10 mL) and extracted with DCM (3×10 mL). The combined organic phase was washed with brine (10 mL), dried with anhydrous Na₂SO₄, filtered and concentrated under vacuum. The residue was purified by column chromatography (33% EtOAc in petrol) to give the title compound as a white solid. Y=81%.

Step 2. Methyl 2-(2-ethoxy-4-isopropyl-7-oxo-thieno [2,3-d]pyridazin-6-yl)acetate. To a solution of 2-(2-ethoxy-4-isopropyl-7-oxo-thieno[2,3-d]pyridazin-6-yl)acetic acid (670 mg, 2.26 mmol) in MeOH (7 mL) at 25° C. was added TMSCl (29 μL, 0.23 mmol). The mixture was stirred at 50° C. for 4 h. The reaction mixture was diluted with H₂O (8 mL) and extracted with EtOAc (3×8 mL). The combined organic layers were washed with brine (8 mL), dried over anhydrous Na₂SO₄, filtered and concentrated under reduced pressure to give a residue. The residue was purified by column chromatography (33% EtOAc in petrol) to give the title compound as a white solid. Y=14%.

Step 3. 2-[2-Ethoxy-7-oxo-4-(propan-2-yl)-6H, 7H-thieno[2,3-d]pyridazin-6-yl]-N-(pyrimidin-2-yl)acetamide. To a solution of methyl 2-(2-ethoxy-4-isopropyl-7-oxo-thieno[2,3-d]pyridazin-6-yl)acetate (100 mg, 0.32 mmol) and pyrimidin-2-amine (46 mg, 0.48 mmol) in THE (1 mL) at 0° C. was added LiHMDS (1 M in THF, 0.71 mL, 0.71 mmol). The mixture was stirred at 25° C. for 12 h. The reaction mixture was diluted with H₂O (1 mL) and extracted with EtOAc (3×1 mL). The combined organic layers were washed with brine (1 mL), dried over anhydrous Na₂SO₄, filtered and concentrated under reduced pressure to give a residue. The residue was purified by prep-HPLC (column: Waters Xbridge Prep OBD C18 150×40 mm×10 μm; mobile phase: [water (NH₄HCO₃)−ACN]; B: 30-60%, 8 min) and lyophilised to give the title compound as a white solid. Y=18%. ¹H NMR (400 MHz, DMSO-d₆) δ 10.93 (s, 1H), 8.68 (d, J=5 Hz, 2H), 7.21 (t, J=5 Hz, 1H), 6.88 (s, 1H), 5.15 (s, 2H), 4.36 (q, J=7 Hz, 2H), 3.29-3.20 (m, 1H), 1.42 (t, J=7 Hz, 3H), 1.25 (d, J=7 Hz, 6H). ¹H NMR (400 MHz, DMSO-d₆+D₂O) δ 8.64 (d, J=5 Hz, 2H), 7.19 (t, J=5 Hz, 1H), 6.82 (s, 1H), 5.10 (s, 2H), 4.32 (q, J=7 Hz, 2H), 3.29-3.20 (m, 1H), 1.38 (t, J=7 Hz, 3H), 1.21 (d, J=7 Hz, 6H). LCMS (ESI): m/z: [M+H]⁺=374.2.

### Compound 108. 2-[2-Methoxy-4-oxo-7-(propan-2-yl)-4H,5H-furo[2,3-d]pyridazin-5-yl]-N-(pyrimidin-2-yl)acetamide

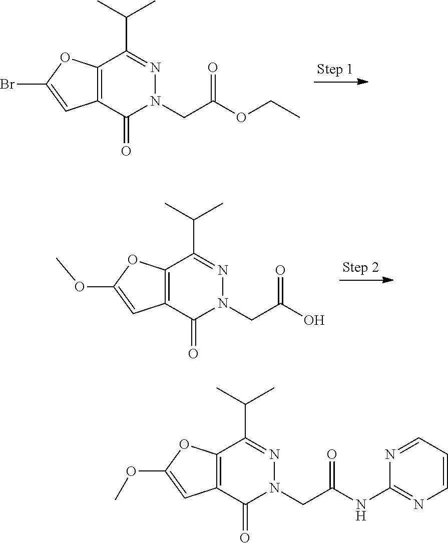

Step 1. 2-(7-Isopropyl-2-methoxy-4-oxo-furo[2,3-d]pyridazin-5-yl)acetic acid. To a solution of ethyl 2-(2-bromo-7-isopropyl-4-oxo-furo[2,3-d]pyridazin-5-yl)acetate (500 mg, 1.46 mmol) in MeOH (5 mL) at 25° C. were added 30% sodium methoxide in MeOH (10.2 mmol) and CuBr (63 mg, 0.44 mmol). The mixture was stirred at reflux for 7 h. The reaction mixture was filtered, and the filtrate was adjusted to pH ˜3 with 2 M HCl. This was concentrated under reduced pressure to give a residue. The residue was purified by prep-HPLC (column: Phenomenex Luna 80×30 mm×3 μm; mobile phase: [water (HCl)−ACN]; B: 20-50%, 8 min) to give the title compound as a white solid. Y=35%. ¹H NMR (400 MHz, DMSO-d₆) δ 12.97 (br. s, 1H), 6.12 (s, 1H), 4.76 (s, 2H), 4.04 (s, 3H), 3.19-3.12 (m, 1H), 1.28-1.26 (m, 6H).

Step 2. 2-[2-Methoxy-4-oxo-7-(propan-2-yl)-4H, 5H-furo[2,3-d]pyridazin-5-yl]-N-(pyrimidin-2-yl)acetamide. To a solution of 2-(7-isopropyl-2-methoxy-4-oxo-furo[2,3-d]pyridazin-5-yl)acetic acid (80 mg, 0.30 mmol) in ACN (0.8 mL) were added NMM (33 μL, 0.30 mmol), COMU (167 mg, 0.39 mmol) and pyrimidin-2-amine (43 mg, 0.45 mmol) at 25° C. The mixture was stirred at 80° C. for 12 h. The reaction mixture was purified by prep-HPLC (column: Waters Xbridge Prep OBD C18 150×40 mm×10 μm; mobile phase: [water (NH₄HCO₃)−ACN]; B: 20-50%, 8 min) and lyophilised to give the title compound as a white solid. Y=14%. ¹H NMR (400 MHz, DMSO-d₆) δ 10.92 (s, 1H), 8.67 (d, J=5 Hz, 2H), 7.20 (t, J=5 Hz, 1H), 6.12 (s, 1H), 5.14 (s, 2H), 4.04 (s, 3H), 3.20-3.11 (m, 1H), 1.28-1.27 (m, J=7 Hz, 6H). ¹H NMR (400 MHz, DMSO-d₆+D₂O) δ 8.65 (d, J=5 Hz, 2H), 7.19 (t, J=5 Hz, 1H), 6.05 (s, 1H), 5.11 (s, 2H), 4.02 (s, 3H), 3.20-3.09 (m, 1H), 1.27-1.25 (m, 6H). LCMS (ESI): m/z: [M+H]⁺=344.1.

### Compound 109. 2-{2-Trideuteromethylamino-7-oxo-4-(propan-2-yl)-6H,7H-thieno[2,3-d]pyridazin-6-yl}-N-(pyrimidin-2-yl)acetamide

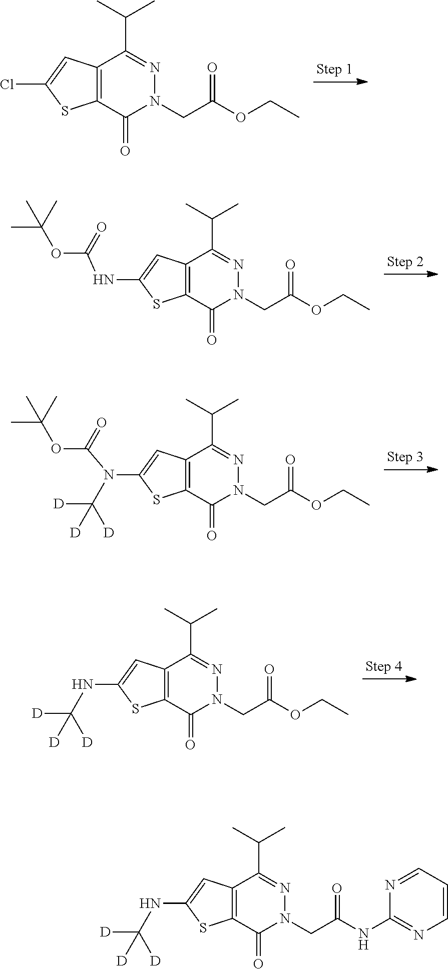

Step 1. Ethyl 2-[2-(tert-butoxycarbonylamino)-4-isopropyl-7-oxo-thieno[2,3-d]pyridazin-6-yl]acetate. To a solution of ethyl 2-(2-chloro-4-isopropyl-7-oxo-thieno[2,3-d]pyridazin-6-yl) acetate (Intermediate D7, 2.0 g, 6.35 mmol) in toluene (20 mL) at 25° C. were added Cs₂CO₃ (4.14 g, 12.7 mmol), tert-butyl carbamate (893 mg, 7.62 mmol), Pd₂(dba)₃ (58 mg, 64 μmol) and (5-diphenylphosphanyl-9,9-dimethyl-xanthen-4-yl)-diphenyl-phosphane (110 mg, 0.19 mmol). The mixture was stirred at 105° C. for 3 h. The reaction mixture was diluted with H₂O (20 mL) and extracted with EtOAc (3×20 mL). The combined organic layers were washed with brine (40 mL), dried over Na₂SO₄, filtered and concentrated under reduced pressure to give a residue. The residue was purified by column chromatography (10-33% EtOAc in petrol) to give the title compound as a yellow solid. Y=44%. ¹H NMR (400 MHz, DMSO-d₆) δ 11.32 (s, 1H), 6.83 (s, 1H), 4.85 (s, 2H), 4.20-4.08 (m, 2H), 3.23-3.13 (m, 1H), 1.51 (s, 9H), 1.26-1.19 (m, 9H).

Step 2. Ethyl 2-[2-[tert-butoxycarbonyl(trideuteriomethyl)amino]-4-isopropyl-7-oxo-thieno[2,3-d]pyridazin-6-yl]acetate. To a solution of ethyl 2-[2-(tert-butoxy carbonyl amino)-4-isopropyl-7-oxo-thieno[2,3-d]pyridazin-6-yl]acetate (500 mg, 1.26 mmol) in DMF (40 mL) and THE (10 mL) at 25° C. were added Cs₂CO₃ (824 mg, 2.53 mmol) and trideuterio(iodo)methane (94 μL, 1.52 mmol). The mixture was stirred at 80° C. for 5 h. The reaction mixture was diluted with H₂O (1 mL) and extracted with EtOAc (3×1 mL). The combined organic layers were washed with brine (1 mL), dried over Na₂SO₄, filtered and concentrated under reduced pressure to give a residue. The residue was purified by column chromatography (10-33% EtOAc in petrol) to give the title compound as a white solid. Y=77%. ¹H NMR (400 MHz, DMSO-d₆) δ 7.06 (s, 1H), 4.87 (s, 2H), 4.24-4.08 (m, 2H), 3.38-3.35 (m, 1H), 1.54 (s, 9H), 1.25 (d, J=7 Hz, 6H), 1.20 (t, J=7 Hz, 3H).

Step 3. Ethyl 2-[4-isopropyl-7-oxo-2-(trideuteriomethylamino) thieno[2,3-d]pyridazin-6-yl]acetate. A mixture ethyl 2-[2-[tert-butoxycarbonyl(trideuteriomethyl)amino]-4-isopropyl-7-oxo-thieno[2,3-d]pyridazin-6-yl]acetate (400 mg, 0.97 mmol) and 4 M HCl in EtOAc (4 mL) was stirred at 25° C. for 1 h. The reaction mixture was concentrated under reduced pressure to give the title compound as yellow oil, used as is.

Step 4. 2-{2-Trideuteromethylamino-7-oxo-4-(propan-2-yl)-6H, 7H-thieno[2,3-d]pyridazin-6-yl}-N-(pyrimidin-2-yl)acetamide. To a solution of ethyl 2-[4-isopropyl-7-oxo-2-(trideuteriomethylamino)thieno[2,3-d]pyridazin-6-yl]acetate (70 mg, 0.20 mmol) and pyrimidin-2-amine (32 mg, 0.34 mmol) in THE (1 mL) at 0° C. was added LiHMDS (1 M in THF, 0.49 mL, 0.49 mmol) and the mixture stirred at 0° C. for 1 h. The mixture was quenched with saturated aqueous NH₄Cl (1 mL) and diluted with H₂O (1 mL). The resulting mixture was extracted with EtOAc (3×1 mL). The combined organic layers were washed with brine (1 mL), dried over anhydrous Na₂SO₄, filtered and concentrated under reduced pressure to give a residue. The residue was purified by prep-HPLC (column: Waters Xbridge Prep OBD C18 150×40 mm×10 μm; mobile phase: [water (NH₄HCO₃)−ACN]; B: 5-45%, 8 min) and lyophilised to give the title compound as a white solid. Y=25%. ¹H NMR (400 MHz, DMSO-d₆) δ 10.87 (s, 1H), 8.67 (d, J=5 Hz, 2H), 7.53 (s, 1H), 7.20 (t, J=5 Hz, 1H), 6.09 (s, 1H), 5.08 (s, 2H), 3.23-3.07 (m, 1H), 1.23 (d, J=7 Hz, 6H). ¹H NMR (400 MHz, DMSO-d₆+D₂O) δ 8.64 (d, J=5 Hz, 2H), 7.50 (s, 0.1H), 7.19 (t, J=5 Hz, 1H), 6.09 (s, 1H), 5.05 (s, 2H), 3.21-3.07 (m, 1H), 1.21 (d, J=7 Hz, 6H). LC-MS (ESI): m/z: [M+H]⁺=362.1.

### Compound 110. 1-[2-(Ethylamino)-7-oxo-4-(propan-2-yl)-6H,7H-thieno[2,3-d]pyridazin-6-yl]-N-(pyrimidin-2-yl)cyclopropane-1-carboxamide

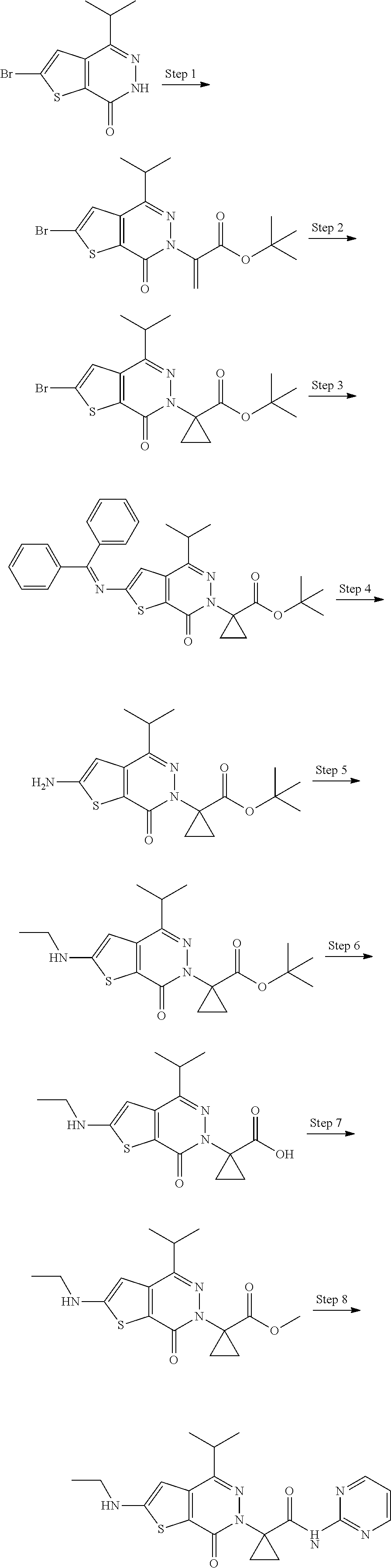

Step 1. Tert-butyl 2-(2-bromo-4-isopropyl-7-oxo-thieno [2,3-d]pyridazin-6-yl)prop-2-enoate. To a solution of 2-bromo-4-isopropyl-6H-thieno[2,3-d]pyridazin-7-one (Intermediate C19, 2.0 g, 7.32 mmol) in DCM (20 mL) at 0° C. under N₂ was added PPh₃ (960 mg, 3.66 mmol) and tert-butyl prop-2-ynoate (1.51 mL, 11.0 mmol). The mixture was stirred at 25° C. for 3 h. The reaction mixture was quenched by addition of H₂O (20 mL) and extracted with DCM (3×20 mL). The combined organic layers were washed with brine (20 mL), dried over Na₂SO₄, filtered and concentrated under reduced pressure to give a residue. The residue was purified by column chromatography (25% EtOAc in petrol) to give the title compound as a white solid. Y=68%.

Step 2. Tert-butyl 1-(2-bromo-4-isopropyl-7-oxo-thieno [2,3-d]pyridazin-6-yl)cyclopropanecarboxylate. To a solution of trimethylsulfoxonium iodide (1.10 g, 5.01 mmol) in DMSO (10 mL) at 25° C. under N₂ was added NaH (60% in mineral oil, 200 mg, 5.01 mmol). The mixture was stirred at 25° C. for 40 min. The mixture was treated with tert-butyl 2-(2-bromo-4-isopropyl-7-oxo-thieno[2,3-d]pyridazin-6-yl)prop-2-enoate (1.0 g, 2.50 mmol), then stirred at 25° C. for 1 h. The reaction mixture was quenched by addition of H₂O (10 mL) and the resulting mixture extracted with EtOAc (3×10 mL). The combined organic layers were washed with brine (10 mL), dried over Na₂SO₄, filtered and concentrated under reduced pressure. The residue was purified by column chromatography (25% EtOAc in petrol) to give the title compound as a white solid. Y=34%. ¹H NMR (400 MHz, DMSO-d₆) δ 7.96 (s, 1H), 3.33-3.31 (m, 1H), 1.72-1.63 (m, 2H), 1.52-1.44 (m, 2H), 1.30 (s, 9H), 1.24 (d, J=7 Hz, 6H).

Step 3. tert-butyl 1-[2-(benzhydrylideneamino)-4-isopropyl-7-oxo-thieno[2,3-d]pyridazin-6-yl]cyclo propanecarboxylate. To a solution of tert-butyl 1-(2-bromo-4-isopropyl-7-oxo-thieno[2,3-d]pyridazin-6-yl)cyclopropanecarboxylate (530 mg, 1.28 mmol) in toluene (5 mL) at 25° C. under N₂ were added Cs₂CO₃ (585 mg, 1.80 mmol), BINAP (80 mg, 128 μmol), Pd(OAc)₂ (29 mg, 128 μmol) and diphenylmethanimine (258 μL, 1.54 mmol). The mixture was stirred at 105° C. for 12 h. The reaction mixture was quenched by addition of H₂O (5 mL) and the resulting mixture extracted with EtOAc (3×5 mL). The combined organic layers were washed with brine (5 mL), dried over anhydrous Na₂SO₄, filtered and concentrated under reduced pressure to give a residue. The residue was purified by column chromatography (25% EtOAc in petrol) to give the title compound as a white solid. Y=62%. ¹H NMR (400 MHz, DMSO-d₆) δ 7.76-7.37 (m, 11H), 3.28-3.19 (m, 1H), 1.67-1.58 (m, 2H), 1.46-1.38 (m, 2H), 1.29 (s, 9H), 1.22 (d, J=7 Hz, 6H).

Step 4. Tert-butyl 1-(2-amino-4-isopropyl-7-oxo-thieno[2,3-d]pyridazin-6-yl) cyclopropanecarboxylate. To a solution of tert-butyl 1-[2-(benzhydrylideneamino)-4-isopropyl-7-oxo-thieno[2,3-d]pyridazin-6-yl]cyclopropanecarboxylate (410 mg, 0.80 mmol) in MeOH (5 mL) at 25° C. under N₂ were added sodium acetate (131 mg, 1.60 mmol) and hydroxylamine hydrochloride (277 mg, 4.0 mmol). The mixture was stirred at 25° C. for 2 h. The reaction mixture was quenched by addition of H₂O (5 mL) and extracted with EtOAc (3×5 mL). The combined organic layers were washed with brine (5 mL), dried over Na₂SO₄, filtered and concentrated under reduced pressure to give a residue. The residue was purified by column chromatography (25% EtOAc in petrol) to give the title compound as a white solid. ¹H NMR (400 MHz, DMSO-d₆) δ 6.97 (s, 2H), 6.06 (s, 1H), 3.13-2.95 (m, 1H), 1.66-1.57 (m, 2H), 1.43-1.35 (m, 2H), 1.31 (s, 9H), 1.21 (d, J=7 Hz, 6H).

Step 5. Tert-butyl 1-[2-(ethylamino)-4-isopropyl-7-oxo-thieno[2,3-d]pyridazin-6-yl]cyclopropanecarboxylate. To a solution of tert-butyl 1-(2-amino-4-isopropyl-7-oxo-thieno[2,3-d]pyridazin-6-yl)cyclo propanecarboxylate (100 mg, 286 μmol) in DCE (3 mL) were added acetaldehyde (40% in water, 60 μL, 0.43 mmol) and acetic acid (41 μL, 0.72 mmol). The mixture was stirred at 25° C. for 1 h, then treated with sodium triacetoxyboranuide (121 mg, 0.57 mmol). The mixture was stirred at 25° C. for a further 1 h. The reaction mixture was quenched by addition of H₂O (1 mL) and extracted with EtOAc (3×1 mL). The combined organic layers were washed with brine (1 mL), dried over anhydrous Na₂SO₄, filtered and concentrated under reduced pressure to give a residue. The residue was purified by prep-TLC (SiO₂, 1:1 petroleum ether/ethyl acetate) to give the title compound as a white solid. Y=56%. ¹H NMR (400 MHz, DMSO-d₆) δ 7.53 (t, J=5 Hz, 1H), 6.06 (s, 1H), 3.20-3.16 (m, 2H), 3.14-3.10 (m, 1H), 1.67-1.58 (m, 2H), 1.43-1.37 (m, 2H), 1.31 (s, 9H), 1.25-1.17 (m, 9H).

Step 6. 1-[2-(Ethylamino)-4-isopropyl-7-oxo-thieno[2,3-d]pyridazin-6-yl]cyclopropanecarboxylic acid. To a solution of tert-butyl 1-[2-(ethylamino)-4-isopropyl-7-oxo-thieno[2,3-d]pyridazin-6-yl]cyclopropanecarboxylate (110 mg, 0.29 mmol) in DCM (0.5 mL) at 25° C. was added TFA (0.5 mL, 6.75 mmol). The mixture was stirred at 25° C. for 12 h. The reaction mixture was diluted with H₂O (1 mL) and the resulting mixture extracted with EtOAc (3×1 mL). The combined organic layers were washed with brine (1 mL), dried over anhydrous Na₂SO₄, filtered and concentrated to give the title compound as a white solid. Y=96%.

Step 7. Methyl 1-[2-(ethylamino)-4-isopropyl-7-oxo-thieno[2,3-d]pyridazin-6-yl]cyclopropane carboxylate. To a solution of 1-[2-(ethylamino)-4-isopropyl-7-oxo-thieno[2,3-d]pyridazin-6-yl]cyclopropanecarboxylic acid (85 mg, 0.26 mmol) in MeOH (2 mL) at 25° C. was added TMSCl (3.4 μL, 26 μmol). The mixture was stirred at 50° C. for 4 h. The reaction mixture was quenched by addition of H₂O (1 mL) and the resulting mixture extracted with EtOAc (3×1 mL). The combined organic layers were washed with brine (1 mL), dried over anhydrous Na₂SO₄, filtered and concentrated under reduced pressure to give the title compound as a white solid. Y=96%.

Step 8. 1-[2-(Ethylamino)-7-oxo-4-(propan-2-yl)-6H, 7H-thieno[2,3-d]pyridazin-6-yl]-N-(pyrimidin-2-yl)cyclopropane-1-carboxamide. To a solution of methyl 1-[2-(ethylamino)-4-isopropyl-7-oxo-thieno[2,3-d]pyridazin-6-yl]cyclopropanecarboxylate (85 mg, 0.25 mmol) in THE (1 mL) at 25° C. was added pyrimidin-2-amine (36 mg, 0.38 mmol). The mixture was cooled to 0° C. and treated with LiHMDS (1 M in THF, 0.56 mL, 0.56 mmol). The mixture was stirred at 25° C. for 12 h under N₂. The reaction mixture was quenched by addition of H₂O (1 mL) and the resulting mixture extracted with EtOAc (3×1 mL). The combined organic layers were washed with brine (1 mL), dried over Na₂SO₄, filtered and concentrated under reduced pressure to give a residue. The residue was purified by prep-HPLC (column: Waters Xbridge Prep OBD C18 150×40 mm×10 μm; mobile phase: [water (NH₄HCO₃)−ACN]; B: 20-50%, 8 min) and lyophilised to give the title compound cyclopropanecarboxamide as a white solid. Y=15%. ¹H NMR (400 MHz, DMSO-d₆) δ 9.81 (s, 1H), 8.65 (d, J=5 Hz, 2H), 7.58 (t, J=5 Hz, 1H), 7.21 (t, J=5 Hz, 1H), 6.11 (s, 1H), 3.24-3.17 (m, 2H), 3.17-3.10 (m, 1H), 1.80-1.72 (m, 2H), 1.45-1.37 (m, 2H), 1.25 (d, J=7 Hz, 6H), 1.20 (t, J=7 Hz, 3H). ¹H NMR (400 MHz, DMSO-d₆+D₂O) δ 8.61 (d, J=5 Hz, 2H), 7.19 (t, J=5 Hz, 1H), 6.11 (s, 1H), 3.17-3.15 (m, 2H), 3.13-3.07 (m, 1H), 1.80-1.70 (m, 2H), 1.48-1.37 (m, 2H), 1.22 (d, J=7 Hz, 6H), 1.17 (t, J=7 Hz, 3H). LCMS (ESI): m/z: [M+H]⁺=399.2.

### Compound 111. 2-[2-(Methylamino)-4-oxo-7-(propan-2-yl)-4H,5H-furo[2,3-d]pyridazin-5-yl]-N-(pyrimidin-2-yl)acetamide

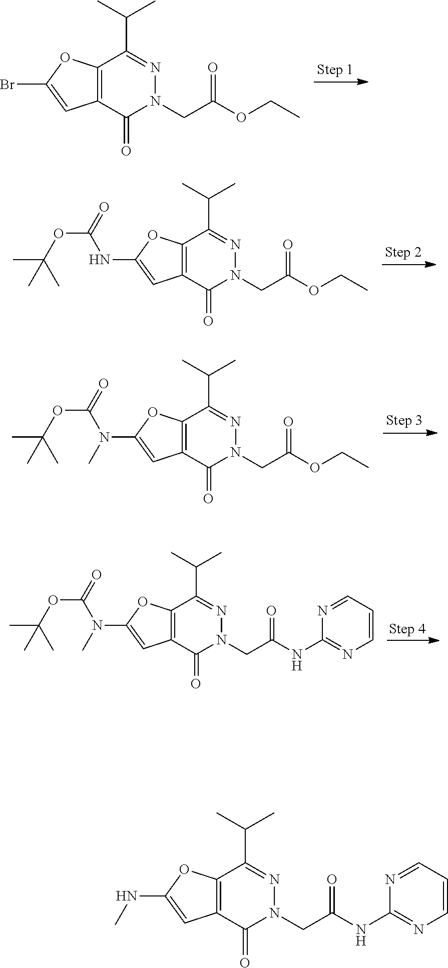

Step 1. Ethyl 2-[2-(tert-butoxycarbonylamino)-7-isopropyl-4-oxo-furo[2,3-d]pyridazin-5-yl]acetate. To a solution of tert-butyl carbamate (205 mg, 1.75 mmol) in toluene (5 mL) at 25° C. were added Cs₂CO₃ (0.95 g, 2.92 mmol), ethyl 2-(2-bromo-7-isopropyl-4-oxo-furo[2,3-d]pyridazin-5-yl)acetate (Intermediate D5, 0.50 g, 1.46 mmol), Pd2(dba)₃ (13 mg, 15 μmol) and Xantphos (25 mg, 44 μmol). The mixture was stirred at 105° C. for 7 h under N₂. The reaction mixture was diluted with H₂O (3 mL) and extracted with DCM (3×5 mL). The combined organic layers were washed with brine (3×5 mL), dried over Na₂SO₄, filtered and concentrated under reduced pressure to give a residue. The residue was purified by prep-TLC (SiO₂, 1:1 petroleum ether/ethyl acetate) to give the title compound as a yellow solid. Y=45%.

Step 2. Ethyl 2-[2-[tert-butoxycarbonyl(methyl)amino]-7-isopropyl-4-oxo-furo[2,3-d]pyridazin-5-yl]acetate. To a solution of ethyl 2-[2-(tert-butoxycarbonylamino)-7-isopropyl-4-oxo-furo[2,3-d]pyridazin-5-yl]acetate (0.20 g, 0.53 mmol) in DMF (16 mL) and THE (4 mL) at 25° C. was added Cs₂CO₃ (515 mg, 1.58 mmol). The mixture was stirred for 0.5 h, then treated with CH₃I (43 μL, 0.69 mmol). The resulting mixture was stirred at 80° C. for 2 h. The reaction mixture was quenched by addition of H₂O (5 mL) and extracted with EtOAc (3×5 mL). The combined organic layers were washed with brine (5×5 mL), dried over Na₂SO₄ filtered and concentrated under reduced pressure to give a residue. The residue was purified by prep-TLC (SiO₂, 1:1 petroleum ether/ethyl acetate) to give the title compound as a white solid. Y=82%. ¹H NMR (400 MHz, DMSO-d₆) δ 6.67 (s, 1H), 4.87 (s, 2H), 4.15 (q, J=7 Hz, 2H), 3.31 (s, 3H), 3.26-3.18 (m, 1H), 1.47 (s, 9H), 1.31 (d, J=7 Hz, 6H), 1.20 (t, J=7 Hz, 3H).

Step 3. Tert-butyl N-[7-isopropyl-4-oxo-5-[2-oxo-2-(pyrimidin-2-ylamino)ethyl]furo[2,3-d]pyridazin-2-yl]-N-methyl carbamate. To a solution of ethyl 2-[2-[tert-butoxycarbonyl(methyl)amino]-7-isopropyl-4-oxo-furo[2,3-d]pyridazin-5-yl]acetate (0.18 g, 0.46 mmol) and pyrimidin-2-amine (52 mg, 0.55 mmol) in THE (2 mL) at 0° C. under N₂ was added LiHMDS (1 M in THF, 1.0 mL, 1.0 mmol). The mixture was stirred at 25° C. under N₂ for 2 h. The reaction mixture was quenched by addition of H₂O (3 mL) at 0° C., and the resulting mixture was extracted with EtOAc (3×3 mL). The combined organic layers were washed with brine (3 mL) dried over Na₂SO₄, filtered and concentrated under reduced pressure to give a residue. The residue was purified by prep-TLC (SiO₂, ethyl acetate) to give the title compound as a white solid. Y=49%. ¹H NMR (400 MHz, DMSO-d₆) δ 10.93 (s, 1H), 8.68 (d, J=5 Hz, 2H), 7.20 (t, J=5 Hz, 1H), 6.68 (s, 1H), 5.16 (s, 2H), 3.31 (s, 3H), 3.25-3.19 (m, 1H), 1.47 (s, 9H), 1.31 (d, J=7 Hz, 6H).

Step 4. 2-[2-(Methylamino)-4-oxo-7-(propan-2-yl)-4H, 5H-furo[2,3-d]pyridazin-5-yl]-N-(pyrimidin-2-yl)acetamide. To a solution of tert-butyl N-[7-isopropyl-4-oxo-5-[2-oxo-2-(pyrimidin-2-ylamino)ethyl]furo[2,3-d]pyridazin-2-yl]-N-methylcarbamate (90 mg, 0.20 mmol) in TFE (1 mL) at 25° C. was added TMSCl (50 uL, 0.39 mmol). The mixture was stirred at 25° C. for 15 min. The reaction mixture was added dropwise into ice-cold saturated Na₂CO₃ aqueous solution and the resulting mixture extracted with EtOAc (3×1 mL). The combined organic layers were washed with brine (1 mL), dried over Na₂SO₄, filtered and concentrated under reduced pressure to give a residue. The residue was purified by prep-HPLC (column: Waters Xbridge Prep OBD C18 150×40 mm×10 μm; mobile phase: [water (NH₄HCO₃)−ACN]; B: 10%-40%, 8 min) and lyophilisation to give the title compound as a white solid. Y=20%. ¹H NMR (400 MHz, DMSO-d₆) δ 10.86 (s, 1H), 8.66 (d, J=5 Hz, 2H), 7.36 (q, J=5 Hz, 1H), 7.19 (t, J=5 Hz, 1H), 5.43 (s, 1H), 5.09 (s, 2H), 3.14-3.06 (m, 1H), 2.78 (d, J=5 Hz, 3H), 1.27 (d, J=7 Hz, 6H). ¹H NMR (400 MHz, DMSO-d₆+D₂O) δ 8.65 (d, J=5 Hz, 2H), 7.19 (t, J=5 Hz, 1H), 5.44 (s, 1H), 5.08 (s, 2H), 3.16-3.04 (m, 1H), 2.78 (s, 3H), 1.26 (d, J=7 Hz, 6H). LCMS (ESI): m/z: [M+H]⁺=343.2.

### Compound 112. 2-[4-Cyclopropyl-2-(methylamino)-7-oxo-6H,7H-thieno[2,3-d]pyridazin-6-yl]-N-(pyrimidin-2-yl)acetamide

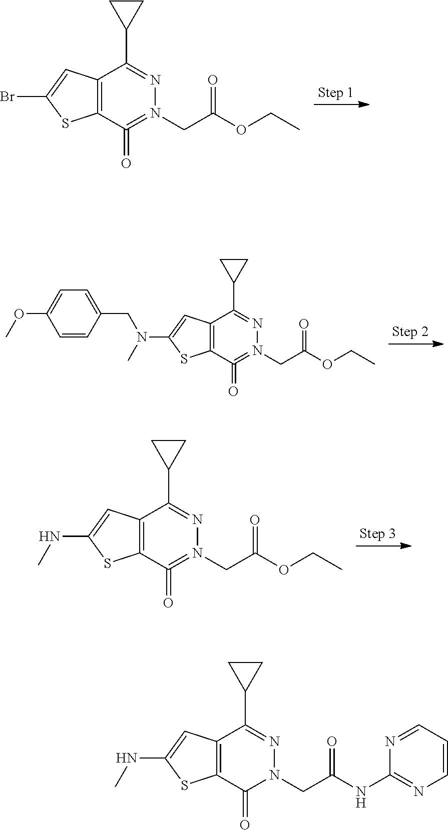

Step 1. Ethyl 2-[4-cyclopropyl-2-[(4-methoxyphenyl) methyl-methyl-amino]-7-oxo-thieno [2,3-d]pyridazin-6-yl]acetate. To a solution of ethyl 2-(2-bromo-4-cyclopropyl-7-oxo-thieno[2,3-d]pyridazin-6-yl)acetate (780 mg, 2.18 mmol) in dioxane (8 mL) at 25° C. under N₂ were added Pd(OAc)₂ (49 mg, 218 μmol), BINAP (136 mg, 218 μmol), Cs₂CO₃ (2.13 g, 6.55 mmol) and 1-(4-methoxyphenyl)-N-methyl-methanamine (660 mg, 4.37 mmol). The mixture was stirred at 105° C. for 3 h. The reaction mixture was diluted with H₂O (8 mL) and extracted with ethyl acetate (3×8 mL). The combined organic layers were washed with brine (8 mL), dried over Na₂SO₄, filtered and concentrated under reduced pressure to give a residue. The residue was purified by column chromatography (50% EtOAc in petrol) to give the title compound as a white solid. Y=43%. ¹H NMR (400 MHz, DMSO-d₆) δ 7.26 (d, J=9 Hz, 2H), 6.93 (d, J=9 Hz, 2H), 6.46 (s, 1H), 4.75 (s, 2H), 4.60 (s, 2H), 4.12 (q, J=7 Hz, 2H), 3.73 (s, 3H), 3.10 (s, 3H), 2.25-2.14 (m, 1H), 1.17 (t, J=7 Hz, 3H), 0.96-0.81 (m, 4H).

Step 2. Ethyl 2-[4-cyclopropyl-2-(methylamino)-7-oxo-thieno [2,3-d]pyridazin-6-yl]acetate. A mixture of ethyl 2-[4-cyclopropyl-2-[(4-methoxyphenyl)methyl-methyl-amino]-7-oxo-thieno[2,3-d]pyridazin-6-yl]acetate (201 mg, 0.47 mmol) and TFA (2 mL) was stirred at 70° C. for 2 h under N₂. The mixture was adjusted to pH ˜9 with saturated aqueous Na₂CO₃ and extracted with ethyl acetate (3×2 mL). The combined organic layers were washed with brine (2 mL), dried over Na₂SO₄, filtered and concentrated under reduced pressure to give a residue. The residue was purified by column chromatography (50% EtOAc in petrol) to give the title compound as a yellow solid. Y=83%. ¹H NMR (400 MHz, DMSO-d₆) δ 7.62 (q, J=5 Hz, 1H), 6.23 (s, 1H), 4.74 (s, 2H), 4.12 (q, J=7 Hz, 2H), 2.86 (d, J=5 Hz, 3H), 2.20-2.14 (m, 1H), 1.17 (t, J=7 Hz, 3H), 0.95-0.89 (m, 2H), 0.86-0.81 (m, 2H).

Step 3. 2-[4-Cyclopropyl-2-(methylamino)-7-oxo-6H, 7H-thieno[2,3-d]pyridazin-6-yl]-N-(pyrimidin-2-yl)acetamide. To a solution of ethyl 2-[4-cyclopropyl-2-(methylamino)-7-oxo-thieno[2,3-d]pyridazin-6-yl]acetate (100 mg, 325 μmol) and pyrimidin-2-amine (46 mg, 0.49 mmol) in THE (1 mL) at 0° C. under N₂ was added LiHMDS (1 M in THF, 0.72 mL, 0.72 mmol). The mixture was stirred at 25° C. for 3 h. The reaction mixture was quenched by addition of H₂O (1 mL) at 0° C. and the resulting mixture extracted with ethyl acetate (3×1 mL). The combined organic layers were washed with brine (1 mL), dried over Na₂SO₄, filtered and concentrated under reduced pressure to give a residue. The residue was purified by prep-HPLC (column: Waters Xbridge Prep OBD C18 150×40 mm×10 μm; mobile phase: [water (NH₄HCO₃)−ACN]; B: 10-50%, 8 min) and lyophilised to give the title compound as a white solid. Y=23%. ¹H NMR (400 MHz, DMSO-d₆) δ 10.85 (s, 1H), 8.66 (d, J=5 Hz, 2H), 7.59 (d, J=5 Hz, 1H), 7.19 (t, J=5 Hz, 1H), 6.23 (s, 1H), 5.02 (s, 2H), 2.87 (d, J=5 Hz, 3H), 2.18-2.15 (m, 1H), 0.92-0.84 (m, 4H). ¹H NMR (400 MHz, DMSO-d₆+D₂O) δ 8.65 (d, J=5 Hz, 2H), 7.19 (t, J=5 Hz, 1H), 6.23 (s, 1H), 5.01 (s, 2H), 2.85 (s, 3H), 2.21-2.09 (m, 1H), 0.95-0.85 (m, 2H), 0.85-0.75 (m, 2H). LCMS (ESI): m/z: [M+H]⁺=357.1.

### Compound 113. 1-[2-(Methylamino)-7-oxo-4-(propan-2-yl)-6H,7H-thieno[2,3-d]pyridazin-6-yl]-N-(pyrimidin-2-yl)cyclopropane-1-carboxamide

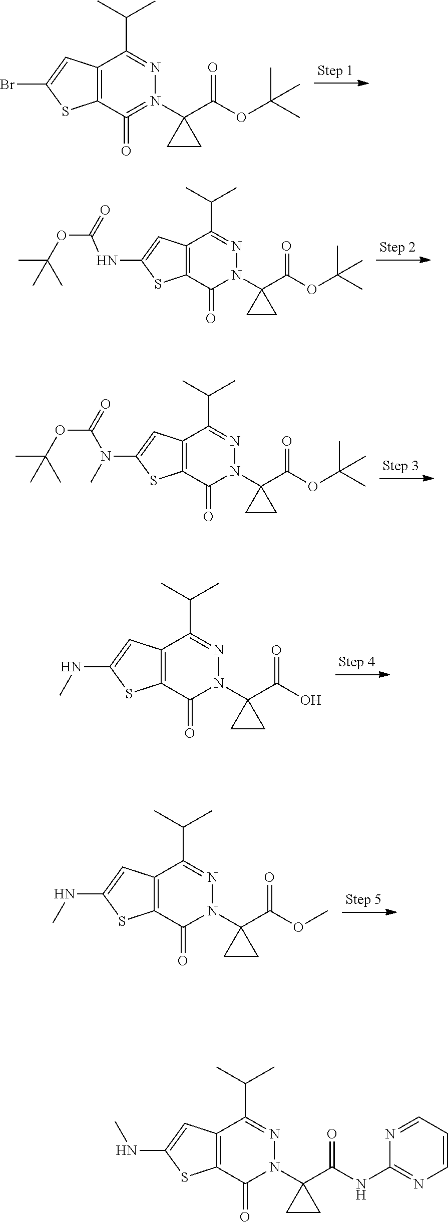

Step 1. Tert-butyl 1-[2-(tert-butoxy carbonylamino)-4-isopropyl-7-oxo-thieno[2,3-d]pyridazin-6-yl]cyclopropanecarboxylate. To a solution of tert-butyl 1-(2-bromo-4-isopropyl-7-oxo-thieno[2,3-d]pyridazin-6-yl)cyclopropanecarboxylate (for synthesis see Compound 110)(100 mg, 0.24 mmol) in toluene (1 mL) at 25° C. under N₂ were added Cs₂CO₃ (158 mg, 0.48 mmol), (5-diphenylphosphanyl-9,9-dimethyl-xanthen-4-yl)-diphenyl-phosphane (4.2 mg, 7.3 μmol), Pd₂(dba)₃ (2.2 mg, 2.4 μmol) and tert-butyl carbamate (34 mg, 0.29 mmol). The mixture was stirred at 90° C. for 3 h. The mixture was diluted with H₂O (1 mL) and extracted with ethyl acetate (3×1 mL), the combined organic layers were washed with brine (1 mL), dried over Na₂SO₄, filtered and concentrated under reduced pressure to give a residue. The residue was purified by column chromatography (25% EtOAc in petrol) to give the title compound as a white solid. Y=46%. ¹H NMR (400 MHz, DMSO-d₆) δ 11.27 (s, 1H), 6.80 (s, 1H), 3.20-3.12 (m, 1H), 1.68-1.62 (m, 2H), 1.51 (s, 9H), 1.47-1.42 (m, 2H), 1.30 (s, 9H), 1.24 (d, J=7 Hz, 6H).

Step 2. Tert-butyl 1-[2-[tert-butoxycarbonyl(methyl)amino]-4-isopropyl-7-oxo-thieno[2,3-d]pyridazin-6-yl]cyclopropanecarboxylate. To a solution of tert-butyl 1-[2-(tert-butoxycarbonylamino)-4-isopropyl-7-oxo-thieno[2,3-d]pyridazin-6-yl]cyclopropanecarboxylate (120 mg, 0.27 mmol) in DMF (12 mL) and THE (3 mL) at 25° C. under N₂ was added Cs₂CO₃ (261 mg, 0.80 mmol). The mixture was stirred at 25° C. for 0.5 h, then treated with Mel (22 μL, 0.35 mmol). The resulting mixture was stirred at 80° C. for 2 h. The mixture was diluted with H₂O (2 mL) and extracted with ethyl acetate (3×2 mL). The combined organic layers were washed with brine (2 mL), dried over Na₂SO₄, filtered and concentrated under reduced pressure to give a residue. The residue was purified by column chromatography (25% EtOAc in petrol) to give the title compound as a white solid. Y=97%.

Step 3. 1-[4-Isopropyl-2-(methylamino)-7-oxo-thieno[2,3-d]pyridazin-6-yl]cyclopropanecarboxylic acid. To a solution of tert-butyl 1-[2-[tert-butoxycarbonyl(methyl)amino]-4-isopropyl-7-oxo-thieno[2,3-d]pyridazin-6-yl]cyclopropanecarboxylate (110 mg, 0.24 mmol) in DCM (0.5 mL) at 25° C. under N₂ was added TFA (0.5 mL). The mixture was stirred at 25° C. for 2 h. The mixture was diluted with H₂O (1 mL) and extracted with ethyl acetate (3×1 mL). The combined organic layers were washed with brine (1 mL), dried over Na₂SO₄, filtered and concentrated under reduced pressure to give a residue. The residue was purified by column chromatography (33% EtOAc in petrol) to give the title compound as a white solid. Y=96%.

Step 4. Methyl 1-[4-isopropyl-2-(methyl amino)-7-oxo-thieno[2,3-d]pyridazin-6-yl]cyclopropanecarboxylate. To a solution of 1-[4-isopropyl-2-(methyl amino)-7-oxo-thieno [2,3-d]pyridazin-6-yl]cyclopropanecarboxylic acid (50 mg, 0.16 mmol) in MeOH (0.5 mL) at 25° C. was added TMSCl (2.1 μL, 16 μmol). The mixture was stirred at 50° C. for 4 h. The mixture was diluted with H₂O (1 mL) and extracted with ethyl acetate (3×1 mL). The combined organic layers were washed with brine (1 mL), dried over Na₂SO₄, filtered and concentrated under reduced pressure to give the title compound as a white solid. Y=96%.

Step 5. 1-[2-(Methylamino)-7-oxo-4-(propan-2-yl)-6H, 7H-thieno[2,3-d]pyridazin-6-yl]-N-(pyrimidin-2-yl)cyclopropane-1-carboxamide. To a solution of methyl 1-[4-isopropyl-2-(methyl amino)-7-oxo-thieno[2,3-d]pyridazin-6-yl]cyclopropanecarboxylate (50 mg, 0.16 mmol) in THE (2.6 mL) at 25° C. under N₂ was added pyrimidin-2-amine (22 mg, 0.23 mmol). The RM was cooled to 0° C. and treated with LiHMDS (1 M in THF, 0.34 mL, 0.34 mmol). The mixture was stirred at 25° C. for 2 h. The reaction mixture was quenched by addition of H₂O (1 mL) at 0° C., and the resulting mixture extracted with ethyl acetate (3×1 mL). The combined organic layers were washed with brine (1 mL), dried over Na₂SO₄, filtered and concentrated under reduced pressure to give a residue. The residue was purified by prep-HPLC (column: Waters Xbridge Prep OBD C18 150×40 mm×10 μm; mobile phase: [water (NH₄HCO₃)−ACN]; B: 25-55%, 8 min) and lyophilised to give the title compound as a white solid. Y=26%. ¹H NMR (400 MHz, DMSO-d₆) δ 9.81 (s, 1H), 8.65 (d, J=5 Hz, 2H), 7.58-7.56 (m, 1H), 7.21 (t, J=5 Hz, 1H), 6.10 (s, 1H), 3.20-3.10 (m, 1H), 2.85 (d, J=5 Hz, 3H), 1.79-1.73 (m, 2H), 1.44-1.38 (m, 2H), 1.26 (d, J=7 Hz, 6H). ¹H NMR (400 MHz, DMSO-d₆+D₂O) δ 8.59 (d, J=5 Hz, 2H), 7.19 (t, J=5 Hz, 1H), 6.10 (s, 1H), 3.12 (q, J=7 Hz, 1H), 2.82 (s, 3H), 1.81-1.71 (m, 2H), 1.48-1.36 (m, 2H), 1.22 (d, J=7 Hz, 6H). LCMS (ESI): m/z: [M+H]⁺=385.2.

### Compound 114. 2-[4-ethyl-2-(methylamino)-7-oxo-6H,7H-thieno[2,3-d]pyridazin-6-yl]-N-(pyrimidin-2-yl)acetamide

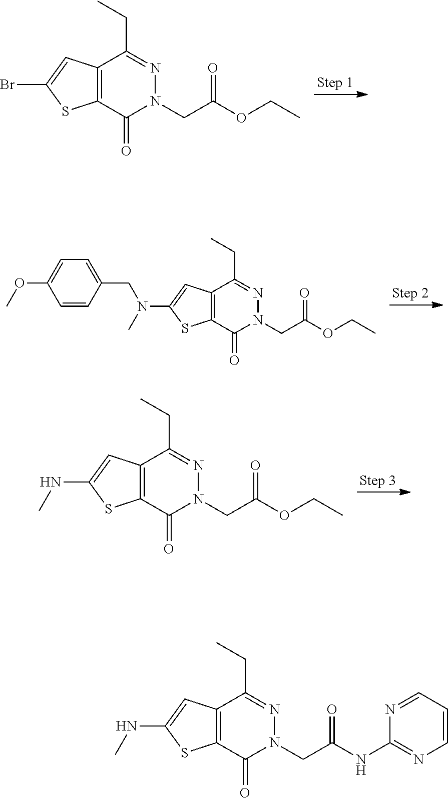

Prepared in an analogous way to Compound 112 using Intermediate D9 to give the title compound as a white solid. ¹H NMR (400 MHz, DMSO-d₆) δ 10.87 (s, 1H), 8.67 (d, J=5 Hz, 2H), 7.58 (q, J=5 Hz, 1H), 7.20 (t, J=5 Hz, 1H), 6.06 (s, 1H), 5.08 (s, 2H), 2.86 (d, J=5 Hz, 3H), 2.74 (q, J=8 Hz, 2H), 1.20 (t, J=8 Hz, 3H). ¹H NMR (400 MHz, DMSO-d₆+D₂O) δ 8.67 (d, J=5 Hz, 2H), 7.19 (t, J=5 Hz, 1H), 6.06 (s, 1H), 5.07 (s, 2H), 2.85 (s, 3H), 2.71 (q, J=8 Hz, 2H), 1.19 (t, J=8 Hz, 3H). LCMS (ESI): m/z: [M+H]⁺=345.1.

### Compound 115. 2-[4-Ethyl-2-(methylamino)-7-oxo-6H,7H-thieno[2,3-d]pyridazin-6-yl]-N-(1,3-oxazol-2-yl)acetamide

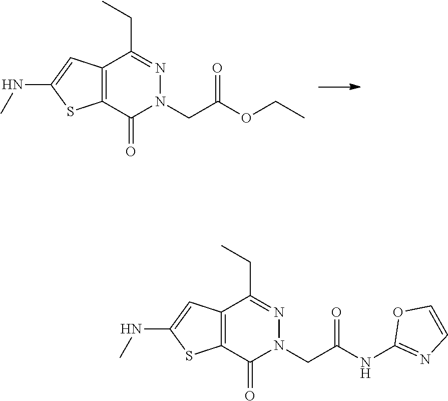

To a solution of ethyl 2-[4-ethyl-2-(methylamino)-7-oxo-thieno[2,3-d]pyridazin-6-yl]acetate (for synthesis see Compound 114)(200 mg, 0.68 mmol) in THE (3 mL) at 25° C. was added oxazol-2-amine (85 mg, 1.02 mmol). The mixture was cooled to 0° C. and treated with LiHMDS (1 M in THF, 1.49 mL, 1.49 mmol). The mixture was stirred at 25° C. for 1 h. The reaction mixture was diluted with H₂O (3 mL) and extracted with EtOAc (3×3 mL). The combined organic layers were washed with brine (3 mL), dried over anhydrous Na₂SO₄, filtered and concentrated under reduced pressure to give a residue. The residue was purified by prep-HPLC (column: Waters Xbridge Prep OBD C18 150×40 mm×10 μm; mobile phase: [water (NH₄HCO₃)−ACN]; B: 10-50%, 8 min) and lyophilised to give the title compound as a white solid. Y=13%. ¹H NMR (400 MHz, DMSO-d₆) δ 11.52 (s, 1H), 7.87 (s, 1H), 7.59 (q, J=5 Hz, 1H), 7.11 (s, 1H), 6.06 (s, 1H), 4.91 (s, 2H), 2.86 (d, J=5 Hz, 3H), 2.74 (q, J=8 Hz, 2H), 1.20 (t, J=8 Hz, 3H). ¹H NMR (400 MHz, DMSO-d₆+D₂O) δ 7.82 (s, 1H), 7.08 (s, 1H), 6.06 (s, 1H), 4.90 (s, 2H), 2.84 (s, 3H), 2.72 (q, J=8 Hz, 2H), 1.18 (t, J=8 Hz, 3H). LCMS (ESI): m/z: [M+H]⁺=334.1.

### Compound 116. 2-{4-Ethyl-2-methyl-7-oxo-6H,7H-thieno[2,3-d]pyridazin-6-yl}-N-(1,3-oxazol-2-yl)acetamide

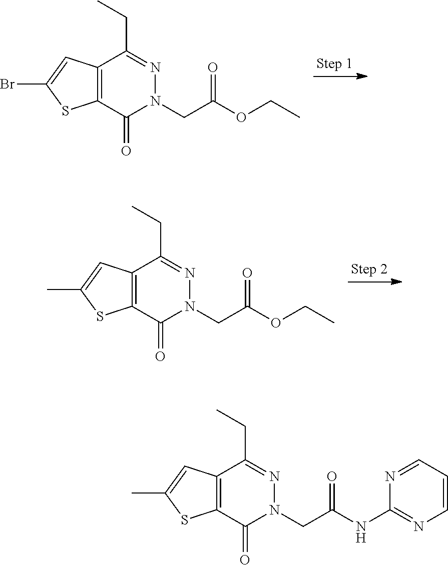

Step 1. Ethyl 2-(4-ethyl-2-methyl-7-oxo-thieno[2,3-d]pyridazin-6-yl)acetate. To a solution of ethyl 2-(2-bromo-4-ethyl-7-oxo-thieno[2,3-d]pyridazin-6-yl)acetate (Intermediate D9, 500 mg, 1.45 mmol) in DME (12.5 mL) and H₂O (2.5 mL) at 25° C. under N₂ were added 2,4,6-trimethyl-1,3,5,2,4,6-trioxatriborinane (405 μL, 2.90 mmol), K₃PO₄ (922 mg, 4.35 mmol) and Pd(dppf)Cl₂·CH₂C₁₂ (59 mg, 72 μmol). The mixture was stirred at 90° C. for 12 h. The reaction mixture was diluted with H₂O (15 mL) and the resulting mixture extracted with EtOAc (3×15 mL). The combined organic layers were washed with brine (15 mL), dried over Na₂SO₄, filtered and concentrated under reduced pressure to give a residue. The residue was purified by column chromatography (50% EtOAc in petrol) to give the title compound as a white solid. Y=15%.

Step 2. 2-{4-Ethyl-2-methyl-7-oxo-6H, 7H-thieno[2,3-d]pyridazin-6-yl}-N-(1,3-oxazol-2-yl)acetamide. To a solution of ethyl 2-(4-ethyl-2-methyl-7-oxo-thieno[2,3-d]pyridazin-6-yl)acetate (50 mg, 0.18 mmol) in THE (2.5 mL) at 25° C. was added pyrimidin-2-amine (25 mg, 0.27 mmol). The mixture was cooled to 0° C. and treated with LiHMDS (1 M in THF, 0.39 mL, 0.39 mmol). The mixture was stirred at 25° C. for 1 h. The reaction mixture was diluted with H₂O (3 mL) and extracted with EtOAc (3×3 mL). The combined organic layers were washed with brine (3 mL), dried over anhydrous Na₂SO₄, filtered and concentrated under reduced pressure to give a residue. The residue was purified by prep-HPLC (column: Waters Xbridge Prep OBD C18 150×40 mm×10 μm; mobile phase: [water (NH₄HCO₃)−ACN]; B: 20-60%, 8 min) and lyophilised to give the title compound as a white solid. Y=9%. ¹H NMR (400 MHz, DMSO-d₆) δ 10.94 (s, 1H), 8.68 (d, J=5 Hz, 2H), 7.39 (s, 1H), 7.20 (t, J=5 Hz, 1H), 5.17 (s, 2H), 2.86 (q, J=8 Hz, 2H), 2.66 (s, 3H), 1.23 (t, J=8 Hz, 3H). ¹H NMR (400 MHz, DMSO-d₆+D₂O) δ 8.68 (d, J=5 Hz, 2H), 7.38 (s, 1H), 7.20 (t, J=5 Hz, 1H), 5.16 (s, 2H), 2.85 (q, J=8 Hz, 2H), 2.65 (s, 3H), 1.22 (t, J=8 Hz, 3H). LCMS (ESI): m/z: [M+H]⁺=330.1.

### Compound 117. 2-{2,4-Dimethyl-7-oxo-6H,7H-thieno[2,3-d]pyridazin-6-yl}-N-(pyrimidin-2-yl)acetamide

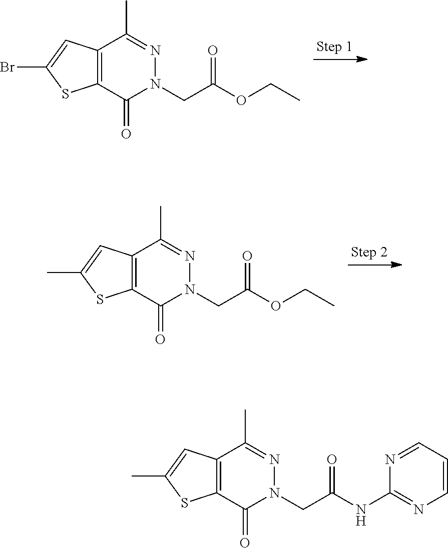

Prepared in an analogous way to Compound 116 using ethyl 2-(2-bromo-4-methyl-7-oxo-thieno [2,3-d]pyridazin-6-yl)acetate (for synthesis see Compound 98) to give the title compound as a white solid. ¹H NMR (400 MHz, DMSO-d₆) δ 10.94 (s, 1H), 8.68 (d, J=5 Hz, 2H), 7.34 (d, J=1 Hz, 1H), 7.21 (t, J=5 Hz, 1H), 5.16 (s, 2H), 2.66 (s, 3H), 2.48 (s, 3H). ¹H NMR (400 MHz, DMSO-d₆+D₂O) δ 8.66 (d, J=5 Hz, 2H), 7.33 (d, J=1 Hz, 1H), 7.20 (t, J=5 Hz, 1H), 5.14 (s, 2H), 2.65 (s, 3H), 2.47 (s, 3H). ¹H NMR (400 MHz, MeOD-d₄) δ 8.63 (d, J=5 Hz, 2H), 7.26 (d, J=1 Hz, 1H), 7.17 (t, J=5 Hz, 1H), 5.28 (s, 2H), 2.70 (s, 3H), 2.55 (s, 3H). LCMS (ESI): m/z: [M+H]⁺=316.0.

### Compound 118. 2-[4-methyl-2-(methylamino)-7-oxo-6H,7H-thieno[2,3-d]pyridazin-6-yl]-N-(pyrimidin-2-yl)acetamide

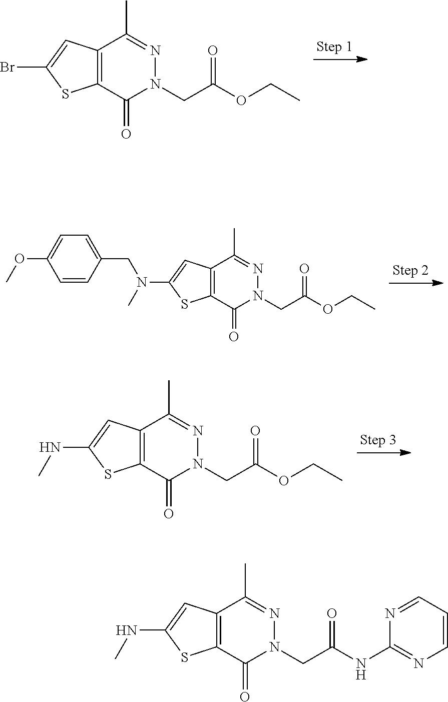

Prepared in an analogous way to Compound 112 using ethyl 2-(2-bromo-4-methyl-7-oxo-thieno [2,3-d]pyridazin-6-yl)acetate (for synthesis see Compound 98) to give the title compound as a white solid. ¹H NMR (400 MHz, DMSO-d₆) δ 10.87 (s, 1H), 8.67 (d, J=5 Hz, 2H), 7.59 (d, J=5 Hz, 1H), 7.20 (t, J=5 Hz, 1H), 6.03 (s, 1H), 5.07 (s, 2H), 2.86 (d, J=5 Hz, 3H), 2.36 (s, 3H). ¹H NMR (400 MHz, DMSO-d₆+D₂O) S 8.64 (d, J=5 Hz, 2H), 7.18 (t, J=5 Hz, 1H), 6.04 (s, 1H), 5.03 (s, 2H), 2.83 (s, 3H), 2.35 (s, 3H). LCMS (ESI): m/z: [M+H]=333.1.

### Compound 119. 2-[4-Methyl-2-(methylamino)-7-oxo-6H,7H-thieno[2,3-d]pyridazin-6-yl]-N-(1,3-oxazol-2-yl)acetamide

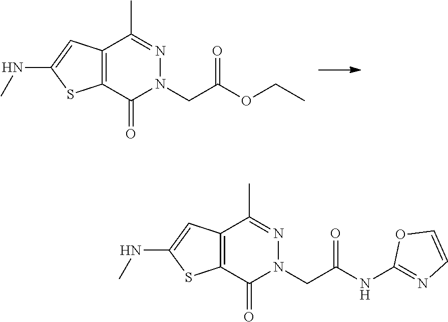

To a solution of ethyl 2-[4-methyl-2-(methylamino)-7-oxo-thieno[2,3-d]pyridazin-6-yl]acetate (for synthesis see Compound 118) (50 mg, 178 μmol) and oxazol-2-amine (22 mg, 0.27 mmol) in THE (1 mL) at 0° C. was added LiHMDS (1 M in THF, 0.39 mL, 0.39 mmol). The resulting solution was stirred at 0° C. for 1 h. The reaction mixture was quenched by addition of H₂O (1 mL) at 0° C., then diluted with H₂O (1 mL). The organics were extracted with EtOAc (3×1 mL), washed with brine (3×1 mL), dried over Na₂SO₄, filtered and concentrated under reduced pressure to give a residue. The residue was purified by prep-HPLC (column: Waters Xbridge Prep OBD C18 150×40 mm×10 μm; mobile phase: [water (NH₄HCO₃)−ACN]; B: 1-30%, 8 min) and lyophilised to give the title compound as a white solid. Y=33%. ¹H NMR (400 MHz, DMSO-d₆) δ 11.52 (s, 1H), 7.87 (s, 1H), 7.61 (q, J=5 Hz, 1H), 7.11 (s, 1H), 6.03 (s, 1H), 4.90 (s, 2H), 2.86 (d, J=5 Hz, 3H), 2.36 (s, 3H). ¹H NMR (400 MHz, DMSO-d₆) δ 7.74 (s, 1H), 7.04 (s, 1H), 6.05 (s, 1H), 4.88 (s, 2H), 2.82 (d, J=5 Hz, 3H), 2.34 (s, 3H). LCMS (ESI): m/z: [M+H]*=320.1.

### Compound 120. 2-[2-(Aminomethyl)-7-oxo-4-(propan-2-yl)-6H,7H-thieno[2,3-d]pyridazin-6-yl]-N-(pyrimidin-2-yl)acetamide

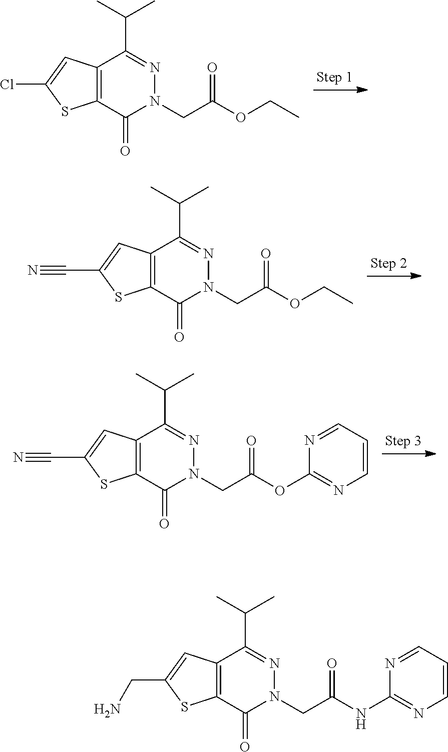

Step 1. Ethyl 2-(2-cyano-4-isopropyl-7-oxo-thieno[2,3-d]pyridazin-6-yl)acetate. To a solution of ethyl 2-(2-chloro-4-isopropyl-7-oxo-thieno[2,3-d]pyridazin-6-yl)acetate (Intermediate D7, 2.0 g, 6.35 mmol) in DMF (20 mL) at 25° C. were added Zn(CN)₂ (887 μL, 14.0 mmol), DPPF (352 mg, 0.64 mmol) and Pd₂(dba)₃ (291 mg, 0.32 mmol). The mixture was stirred at 140° C. for 2 h. The reaction mixture was quenched by H₂O (20 mL) at 0° C. and the resulting mixture extracted with EtOAc (3×20 mL). The combined organic layers were washed with brine (20 mL), dried over Na₂SO₄, filtered and concentrated under reduced pressure to give a residue. The residue was purified by column chromatography (50% EtOAc in petrol) to give the title compound as a white solid. Y=52%. ¹H NMR (400 MHz, DMSO-d₆) δ 8.73 (s, 1H), 4.95 (s, 2H), 4.16 (q, J=7 Hz, 2H), 3.45-3.36 (m, 1H), 1.35-1.16 (m, 9H).

Step 2. 2-(2-Cyano-4-isopropyl-7-oxo-thieno[2,3-d]pyridazin-6-yl)-N-pyrimidin-2-yl-acetamide. To a solution of ethyl 2-(2-cyano-4-isopropyl-7-oxo-thieno[2,3-d]pyridazin-6-yl)acetate (0.60 g, 1.96 mmol) and pyrimidin-2-amine (280 mg, 2.95 mmol) in THE (6 mL) at 0° C. under N₂ was added LiHMDS (1 M in THF, 4.32 mL, 4.32 mmol). The mixture was stirred at 25° C. for 2 h. The reaction mixture was diluted with H₂O (8 mL) and extracted with EtOAc (3×8 mL). The combined organic layers were washed with brine (8 mL), dried over Na₂SO₄, filtered and concentrated under reduced pressure to give a residue. The residue was purified by prep-HPLC (column: Waters Xbridge BEH C18 250×50 mm×10 μm; mobile phase: [water (NH₄HCO₃)−ACN]; B: 20-50%, 10 min) and lyophilised to give the title compound as a white solid. Y=43%. ¹H NMR (400 MHz, DMSO-d₆) δ 11.00 (s, 1H) 8.84-8.58 (m, 3H) 7.21 (t, J=5 Hz, 1H) 5.23 (s, 2H) 3.45-3.36 (m, 1H) 1.27 (d, J=7 Hz, 6H).

Step 3. 2-[2-(Aminomethyl)-7-oxo-4-(propan-2-yl)-6H,7H-thieno[2,3-d]pyridazin-6-yl]-N-(pyrimidin-2-yl)acetamide. To a solution of 2-(2-cyano-4-isopropyl-7-oxo-thieno[2,3-d]pyridazin-6-yl)-N-pyrimidin-2-yl-acetamide (200 mg, 0.56 mmol) in EtOH (10 mL) under N₂ were added Raney-Ni (100 mg) and 25% NH₃·H₂O (1.74 mL, 11.3 mmol). The suspension was degassed and purged with H₂ 3 times. The mixture was stirred under H₂ (15 Psi) at 25° C. for 2 h. The reaction mixture was filtered and the filtrated was concentrated under reduced pressure to give a residue. The residue was purified by prep-HPLC (column: Waters Xbridge Prep OBD C18 150×40 mm×10 μm; mobile phase: [water (NH₄HCO₃)−ACN]; B: 10-40%, 8 min) and lyophilised to give the title compound as a white solid. Y=3%. ¹H NMR (400 MHz, DMSO-d₆) δ 10.80 (br. s, 1H), 8.68 (d, J=5 Hz, 2H), 7.50 (s, 1H), 7.21 (t, J=5 Hz, 1H), 5.17 (s, 2H), 4.06 (s, 2H), 3.30-3.25 (m, 1H), 1.27 (d, J=7 Hz, 6H). ¹H NMR (400 MHz, DMSO-d₆+D₂O) δ 8.66 (d, J=5 Hz, 2H), 7.49 (s, 1H), 7.20 (t, J=5 Hz, 1H), 5.15 (s, 2H), 4.04 (s, 2H), 3.23-3.34 (m, 1H), 1.19-1.32 (m, 6H). LCMS (ESI): m/z [M+H]⁺=359.1.

### Biological Activity of Exemplary Compounds

The biological activity of the compounds of the present disclosure was determined utilising the assay described herein.

PBMC IC₅₀ determination assay. The compounds of the present disclosure were tested for their inhibitory activity against IL-1β release upon NLRP3 activation in peripheral blood mononuclear cells (PBMC).

Protocol A. PBMC were isolated from buffy coats by density gradient centrifugation on Histopaque-1077 (Sigma, cat no. 10771). Isolated cells were seeded into the wells of a 96-well plate and incubated for 3 h with lipopolysaccharide (LPS). Following medium exchange, the compounds of the present disclosure were added (a single compound per well) and the cells were incubated for 30 min. Next, the cells were stimulated either with ATP (5 mM) or nigericin (10 μM) for 1 h and the cell culture media from the wells were collected for further analysis.

The release of IL-1β into the media was determined by a quantitative detection of IL-1β in the media using an IL-1β enzyme-linked immunosorbent assay (ELISA) Ready-SET-Go!, eBioscience cat. No. 88-7261-88. Briefly, in a first step, high affinity binding plates (Corning, Costar 9018 or NUNC Maxisorp Cat No. 44-2404) were coated overnight at 4° C. with specific capture antibody included in the kit (anti-human IL-1β ref. 14-7018-68). Subsequently, plates were blocked with blocking buffer for 1 h at room temperature (rt) and after washing with a buffer (PBS with 0.05% Tween-20) incubated with protein standard and culture media. After 2 h of incubation at rt, plates were washed and incubated with biotinylated detection antibody included in the kit (anti-human IL-1β Biotin ref 33-7110-68) for 1 h at rt. Plates were washed and incubated with HRP-streptavidin for 30 min at rt and washed again. The signal was developed after addition of 3,3′,5,5′-tetramethylbenzidine-peroxidase (TMB) until colour appeared and the reaction was stopped by 2 M H₂SO₄. A microplate spectrophotometer (BioTek) was used to detect signals with 450 nm. The detection range of IL-1β ELISA was 2-150 ng/mL.

Protocol B. PBMC were isolated from buffy coats by density gradient centrifugation on Histopaque-1077 (Sigma, cat no. 10771). Isolated cells were seeded into the wells (280,000 cells/well) of a 96-well plate and incubated for 3 h with lipopolysaccharide (LPS, 1 μg/mL diluted 1000× from a 1 mg/mL stock solution). The compounds of the present disclosure were added (a single compound per well) and the cells were incubated for 30 min. Next, the cells were stimulated with ATP (5 mM final concentration diluted 20× from a 100 mM stock solution) for 1 h and the cell culture media from the wells were collected for further analysis.

The release of IL-1β into the media was determined by quantitative detection of IL-1β in the media using HTRF®, CisBio cat. No. 62HIL1BPEH. Briefly, cell culture supernatant were dispensed directly into the assay plate containing antibodies labelled with the HTRF® donor and acceptor. A microplate spectrophotometer (BMG) was used to detect signals at 655 nm and 620 nm. The detection range of IL-1β HTRF® was 39-6500 μg/mL.

The determination of the IC₅₀ values was preformed using the Graph Pad Prism software and the measured IC₅₀ values of compounds of the present disclosure are shown in Table A below (“+++++” means≤0.1 μM; “++++” means≥0.1 μM and ≤1 μM; “+++” means≥1 and <3 μM; “++” means≥3 and ≤10 μM; “+” means≥10 and <50 μM). These results show that the compounds of the present disclosure are capable of inhibiting IL-1β release upon inflammasome activation.

TABLE A

| Compound | PBMC IC₅₀, |
|---|---|
| No | μM |
| 1 | ++ |
| 2 | +++ |
| 3 | ++ |
| 4 | ++ |
| 5 | +++ |
| 6 | + |
| 7 | +++ |
| 8 | ++++ |
| 9 | + |
| 10 | +++ |
| 11 | +++ |
| 12 | ++++ |
| 13 | + |
| 14 | + |
| 15 | ++++ |
| 16 | ++++ |
| 17 | ++++ |
| 18 | + |
| 19 | + |
| 20 | + |
| 21 | + |
| 22 | + |
| 23 | ++++ |
| 24 | +++++ |
| 25 | +++ |
| 26 | ++++ |
| 27 | ++++ |
| 28 | ++++ |
| 29 | ++++ |
| 30 | ++ |
| 31 | +++ |
| 32 | ++++ |
| 33 | ++++ |
| 34 | +++ |
| 35 | ++++ |
| 36 | +++++ |
| 40 | +++ |
| 42 | ++++ |
| 43 | ++++ |
| 46 | +++ |
| 47 | ++++ |
| 50 | +++ |
| 51 | ++++ |
| 54 | ++++ |
| 55 | ++++ |
| 59 | ++++ |
| 60 | ++++ |
| 61 | +++ |
| 62 | ++++ |
| 63 | ++++ |
| 64 | ++++ |
| 65 | ++++ |
| 66 | ++ |
| 67 | ++++ |
| 68 | +++ |
| 69 | ++++ |
| 70 | + |
| 71 | +++ |
| 72 | ++++ |
| 73 | ++++ |
| 74 | +++ |
| 75 | ++++ |
| 76 | ++++ |
| 77 | +++++ |
| 78 | +++++ |
| 79 | ++++ |
| 80 | ++++ |
| 81 | ++++ |
| 82 | ++++ |
| 83 | ++++ |
| 84 | ++++ |
| 85 | +++ |
| 86 | ++++ |
| 87 | +++ |
| 88 | ++++ |
| 89 | ++++ |
| 90 | ++++ |
| 91 | ++++ |
| 92 | ++++ |
| 93 | ++++ |
| 94 | +++++ |
| 95 | ++++ |
| 96 | + |
| 97 | ++ |
| 98 | +++ |
| 99 | ++++ |
| 100 | +++ |
| 101 | +++ |
| 102 | ++++ |
| 103 | +++ |
| 104 | ++++ |
| 105 | ++++ |
| 106 | ++++ |
| 107 | +++ |
| 108 | ++ |
| 109 | +++++ |
| 110 | ++ |
| 111 | ++++ |
| 112 | ++++ |
| 113 | + |
| 114 | ++++ |
| 115 | ++++ |
| 116 | +++ |
| 117 | +++ |
| 118 | +++ |
| 119 | +++ |
| 120 | + |

### EQUIVALENTS

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The foregoing description has been presented only for the purposes of illustration and is not intended to limit the disclosure to the precise form disclosed, but by the claims appended hereto.

1. A compound of Formula (III): 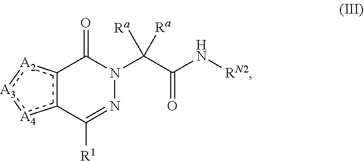
or a prodrug, solvate, or pharmaceutically acceptable salt thereof, wherein:
each [symbol] is independently a single bond or double bond as valency permits;
A₂ is CR², N, NR^{2a}, O, or S, as valency allows;
A₃ is CR², N, NR^{2a}, O, or S, as valency allows;
A₄ is CR², N, NR^{2a}, O, or S, as valency allows,
wherein at least one of A₂, A₃, or A₄ is N, NR^{2a}, O, or S, wherein when A₂ is S, A₄ is CR², NR^{2a}, O, or S;
R¹ is H, —N(C₁-C₆ alkyl)₂, C₁-C₆ alkyl, C₂-C₆ alkenyl, or C₃-C₁₂ cycloalkyl, wherein the —N(C₁-C₆ alkyl)₂, C₁-C₆ alkyl, C₂-C₆ alkenyl, or C₃-C₁₂ cycloalkyl is optionally substituted with one or more R^{1S};
each R^{1S} independently is halogen, cyano, —OH, or C₁-C₆ alkyl;
each R² independently is H, halogen, cyano, —OH, —NH₂, —NO₂, —C(═O)NH₂, C₁-C₆ alkyl, C₂-C₆ alkenyl, C₂-C₆ alkynyl, —O(C₁-C₆ alkyl), —NH(C₁-C₆ alkyl), —N(C₁-C₆ alkyl)₂, C₃-C₁₂ cycloalkyl, 3- to 12-membered heterocycloalkyl, C₆-C₁₀ aryl, or 5- to 10-membered heteroaryl, wherein the C₁-C₆ alkyl, C₂-C₆ alkenyl, C₂-C₆ alkynyl, —O(C₁-C₆ alkyl), —NH(C₁-C₆ alkyl), —N(C₁-C₆ alkyl)₂, C₃-C₁₂ cycloalkyl, 3- to 12-membered heterocycloalkyl, C₆-C₁₀ aryl, or 5- to 10-membered heteroaryl is optionally substituted with one or more R^{2S},
or two R² together with the atoms to which they are attached form a C₃-C₁₂ cycloalkyl or 3- to 12-membered heterocycloalkyl, wherein the C₃-C₁₂ cycloalkyl or 3- to 12-membered heterocycloalkyl is optionally substituted with one or more R^{2S};
each R^{2S} independently is halogen, —OH, —O(C₁-C₆ alkyl), —NH₂, —NH(C₁-C₆ alkyl), —N(C₁-C₆ alkyl)₂, or C₃-C₁₂ cycloalkyl;
each R^{2a} independently is H, C₁-C₆ alkyl, C₂-C₆ alkenyl, C₂-C₆ alkynyl, C₁-C₆ haloalkyl, —(CH₂)₀₋₃—C₃-C₁₂ cycloalkyl, or —(CH₂)₀₋₃-(3- to 12-membered heterocycloalkyl);
each R^{a} independently is H or C₁-C₆ alkyl; or two R^{a}, together with the atom they attach to, form C₃-C₁₂ cycloalkyl;
R^{N2} is C₁-C₆ alkyl, C₂-C₆ alkenyl, C₂-C₆ alkynyl, —O—(C₁-C₆ alkyl), —O—(C₂-C₆ alkenyl), —O—(C₂-C₆ alkynyl), —NH—(C₁-C₆ alkyl), —NH—(C₂-C₆ alkenyl), —NH—(C₂-C₆ alkynyl), C₃-C₁₂ cycloalkyl, 3- to 12-membered heterocycloalkyl, C₆-C₁₀ aryl, 5- to 10-membered heteroaryl, —(C₁-C₆ alkyl)-(C₃-C₁₂ cycloalkyl), —(C₁-C₆ alkyl)-(3- to 12-membered heterocycloalkyl), —(C₁-C₆ alkyl)-(C₆-C₁₀ aryl), or —(C₁-C₆ alkyl)-(5- to 10-membered heteroaryl); wherein the C₁-C₆ alkyl, C₂-C₆ alkenyl, C₂-C₆ alkynyl, —O—(C₁-C₆ alkyl), —O—(C₂-C₆ alkenyl), —O—(C₂-C₆ alkynyl), —NH—(C₁-C₆ alkyl), —NH—(C₂-C₆ alkenyl), —NH—(C₂-C₆ alkynyl), C₃-C₁₂ cycloalkyl, 3- to 12-membered heterocycloalkyl, C₆-C₁₀ aryl, 5- to 10-membered heteroaryl, —(C₁-C₆ alkyl)-(C₃-C₁₂ cycloalkyl), —(C₁-C₆ alkyl)-(3- to 12-membered heterocycloalkyl), —(C₁-C₆ alkyl)-(C₆-C₁₀ aryl), or —(C₁-C₆ alkyl)-(5- to 10-membered heteroaryl) is optionally substituted with one or more R^{N2a};
each R^{N2a} independently is oxo, halogen, cyano, —OH, —NH₂, —C(═O)H, —C(═O)OH, C₁-C₆ alkyl, C₂-C₆ alkenyl, C₂-C₆ alkynyl, —O(C₁-C₆ alkyl), —NH(C₁-C₆ alkyl), —N(C₁-C₆ alkyl)₂, —C(═O)(C₁-C₆ alkyl), —C(═O)O(C₁-C₆ alkyl), —NHC(═O)O(C₁-C₆ alkyl), —S(═O)₂(C₁-C₆ alkyl), —S(═O)₂N(C₁-C₆ alkyl)₂, C₃-C₁₂ cycloalkyl, 3- to 12-membered heterocycloalkyl, C₆-C₁₀ aryl, 5- to 10-membered heteroaryl, —(C₁-C₆ alkyl)-(C₃-C₁₂ cycloalkyl), —(C₁-C₆ alkyl)-(3- to 12-membered heterocycloalkyl), —(C₁-C₆ alkyl)-(C₆-C₁₀ aryl), or —(C₁-C₆ alkyl)-(5- to 10-membered heteroaryl); wherein the C₁-C₆ alkyl, C₂-C₆ alkenyl, C₂-C₆ alkynyl, —O(C₁-C₆ alkyl), —NH(C₁-C₆ alkyl), —N(C₁-C₆ alkyl)₂, —C(═O)(C₁-C₆ alkyl), —C(═O)O(C₁-C₆ alkyl), —NHC(═O)O(C₁-C₆ alkyl), —S(═O)₂(C₁-C₆ alkyl), —S(═O)₂N(C₁-C₆ alkyl)₂, C₃-C₁₂ cycloalkyl, 3- to 12-membered heterocycloalkyl, C₆-C₁₀ aryl, 5- to 10-membered heteroaryl, —(C₁-C₆ alkyl)-(C₃-C₁₂ cycloalkyl), —(C₁-C₆ alkyl)-(3- to 12-membered heterocycloalkyl), —(C₁-C₆ alkyl)-(C₆-C₁₀ aryl), or —(C₁-C₆ alkyl)-(5- to 10-membered heteroaryl) is optionally substituted with one or more R^{N2ab}; and
each R^{N2ab} independently is oxo, halogen, cyano, —OH, —NH₂, —C(═O)H, —C(═O)OH, —O(C₁-C₆ alkyl), —NH(C₁-C₆ alkyl), —N(C₁-C₆ alkyl)₂, —C(═O)(C₁-C₆ alkyl), —C(═O)O(C₁-C₆ alkyl), —NHC(═O)O(C₁-C₆ alkyl), —S(═O)₂(C₁-C₆ alkyl), or —S(═O)₂N(C₁-C₆ alkyl)₂.

2. The compound of claim 1, wherein:
R¹ is C₁-C₆ alkyl, C₂-C₆ alkenyl, or C₃-C₇ cycloalkyl, wherein the C₁-C₆ alkyl is optionally substituted with one or more R^{1S};
each R^{1S} independently is halogen;
each R² independently is H, halogen, cyano, —NH₂, C₁-C₆ alkyl, —O(C₁-C₆ alkyl), —NH(C₁-C₆ alkyl), —N(C₁-C₆ alkyl)₂, C₃-C₁₂ cycloalkyl, wherein the C₁-C₆ alkyl and —NH(C₁-C₆ alkyl) is optionally substituted with one or more R^{2S},
each R^{2S} independently is halogen, —O(C₁-C₆ alkyl or —NH₂;
each R^{2a} independently is C₁-C₆ alkyl or —(CH₂)₀₋₃—C₃-C₁₂ cycloalkyl;
R^{N2} is C₃-C₁₂ cycloalkyl, 3- to 12-membered heterocycloalkyl or 5- to 10-membered heteroaryl, wherein the C₃-C₁₂ cycloalkyl, 3- to 12-membered heterocycloalkyl or 5- to 10-membered heteroaryl is optionally substituted with one or more R^{N2a}; and
each R^{N2a} independently is halogen, cyano, —OH, C₁-C₆ alkyl, C₃-C₁₂ cycloalkyl, —C(═O)O(C₁-C₆ alkyl), wherein the C₁-C₆ alkyl is optionally substituted with one or more R^{N2ab}; and
each R^{N2ab} independently is —C(═O)O(C₁-C₆ alkyl).

3. The compound of claim 1 or claim 2, wherein:
A₂ is S, A₃ is CR², and A₄ is CR²; or
A₂ is CR², A₃ is CR², and A₄ is S; or
A₂ is N, A₃ is NR^{2a}, and A₄ is CR²; or
A₂ is O, A₃ is CR², and A₄ is CR²; or
A₂ is NR^{2a}, A₃ is N, and A₄ is CR²; or
A₂ is N, A₃ is CR², and A₄ is NR^{2a}; or
A₂ is CR², A₃ is N, and A₄ is NR^{2a}; or
A₂ is CR², A₃ is NR^{2a}, and A₄ is N; or
A₂ is CR², A₃ is CR², and A₄ is O.

4. The compound of any one of the preceding claims, wherein R² independently is H, halogen, cyano, —NH₂, C₁-C₆ alkyl —O(C₁-C₆ alkyl), —NH(C₁-C₆ alkyl), —N(C₁-C₆ alkyl)₂, C₃-C₁₂ cycloalkyl, wherein the C₁-C₆ alkyl and —NH(C₁-C₆ alkyl) is optionally substituted with one or more R^{2S}.

5. The compound of any one of the preceding claims, wherein R² independently is H, halogen (such as chlorine or bromine), cyano, —NH₂, C₁-C₆ alkyl (such as methyl, ethyl, or propyl), —O(C₁-C₆ alkyl) (such as —O-methyl or —O-ethyl), —NH(C₁-C₆ alkyl) (such as —NH-methyl, —NH-CD₃, —NH-ethyl, or —NH-isopropyl), —N(C₁-C₆ alkyl)₂ (such as (—N(Me)(Et)), C₃-C₁₂ cycloalkyl (such as cyclopropyl), wherein the C₁-C₆ alkyl and —NH(C₁-C₆ alkyl) is optionally substituted with one or more R^{2S} (such as —CH₂—CF₃, —NHCH₂CHF₂, —CH₂—O-methyl, —NHCH₂CH₂OMe or —CH₂—NH₂).

6. The compound of any one of claims 1 to 4, wherein R² independently is H, chlorine, bromine, cyano, —NH₂, methyl, ethyl, propyl, —O-methyl, —O-ethyl, —NH-methyl, —NH-CD₃, —NH— ethyl, —NH-isopropyl, —N(Me)(Et), cyclopropyl, —CH₂—CF₃, —NHCH₂CHF₂, —CH₂—O-methyl, —NHCH₂CH₂OMe or —CH₂—NH₂.

7. The compound of any one of the preceding claims, wherein each R^{2a} independently is C₁-C₆ alkyl or —(CH₂)₀₋₃—C₃-C₁₂ cycloalkyl.

8. The compound of any one of claims 1 to 6, wherein each R^{2a} independently is C₁-C₆ alkyl (such as methyl, ethyl, isopropyl) or —(CH₂)₀₋₃—C₃-C₁₂ cycloalkyl (such as cyclopropyl or cyclobutyl).

9. The compound of any one of claims 1 to 6, wherein each R^{2a} independently is methyl, ethyl, isopropyl, cyclopropyl or cyclobutyl.

10. The compound of any one of the preceding claims, wherein R¹ is C₁-C₆ alkyl, C₂-C₆ alkenyl or C₃-C₇ cycloalkyl, wherein C₁-C₆ alkyl is optionally substituted with one or more R^{1S}.

11. The compound of any one of claims 1 to 9, wherein R¹ is C₁-C₆ alkyl (such as methyl, ethyl or isopropyl), C₂-C₆ alkenyl (such as isopropenyl), C₃-C₇ cycloalkyl (such as cyclopropyl) or C₆ alkyl is optionally substituted with one or more R^{1S} (such as fluoromethyl).

12. The compound of any one of claims 1 to 9, wherein R¹ is methyl, ethyl, isopropyl, isopropenyl, cyclopropyl or fluoromethyl.

13. The compound of any one of the preceding claims, wherein both R^{a} are H or two R^{a}, together with the atom they attach to, form C₃-C₁₂ cycloalkyl.

14. The compound of any one of claims 1 to 12, wherein both R^{a} are H or two R^{a}, together with the atom they attach to, form C₃-C₇ cycloalkyl.

15. The compound of any one of claims 1 to 12, wherein both R^{a} are H or two R^{a}, together with the atom they attach to, form, C₃-C₆ cycloalkyl.

16. The compound of any one of claims 1 to 12, wherein both R^{a} are H or two R^{a}, together with the atom they attach to, form, cyclopropyl.

17. The compound of any one of the preceding claims, wherein R^{N2} is C₃-C₁₂ cycloalkyl (such as cyclobutyl), 3- to 12-membered heterocycloalkyl (such as piperidinyl, octahydroindolizin-8-yl, or oxaspiro[3.3]heptan-6-yl) or 5- to 10-membered heteroaryl (such as oxazolyl, pyrimidinyl or triazolylpyridinyl), wherein the C₃-C₁₂ cycloalkyl, 3- to 12-membered heterocycloalkyl or 5- to 10-membered heteroaryl is optionally substituted with one or more R^{N2a}.

18. The compound of any one of claims 1 to 16, wherein R^{N2} is cyclobutyl, piperidinyl, octahydroindolizin-8-yl, oxaspiro[3.3]heptan-6-yl), oxazolyl, pyrimidinyl or triazolylpyridinyl, each of which is optionally substituted with one or more R^{N2a}.

19. The compound of any one of the preceding claims, wherein R^{N2a} independently is halogen (such as F or Cl), cyano, —OH, C₁-C₆ alkyl (such as methyl), C₃-C₁₂ cycloalkyl (such as cyclopropyl or cyclobutyl), —C(═O)O(C₁-C₆ alkyl) (such as —COO-ethyl), wherein the C₁-C₆ alkyl (such as methyl) is optionally substituted with one or more R^{N2ab} (such as —C(═O)O(C₁-C₆ alkyl), in particular —C(═O)O(ethyl)).

20. The compound of any one of the preceding claims, which is a compound of Formula (III-a), (III-b), (III-c), (III-d), (III-e), (III-f), or (III-g): 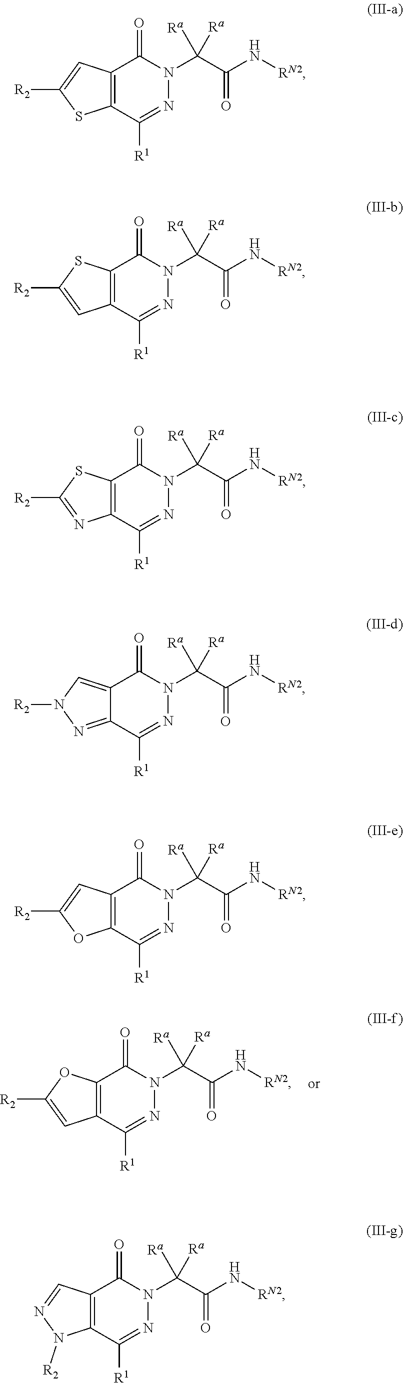
or a prodrug, solvate, or pharmaceutically acceptable salt thereof.

21. The compound of any one of claims 1 to 19, which is a compound of Formula (III-b), Formula (III-d), or Formula (III-e).

22. The compound of any one of claims 1 to 19, which is a compound of Formula (III-e).

23. The compound of any one of the preceding claims, wherein the compound is selected from the compounds described in Table 1 and prodrugs and pharmaceutically acceptable salts thereof.

24. A compound being an isotopic derivative of the compound of any one of the preceding claims.

25. A process for preparing a compound of Formula (III) of any one of the preceding claims which comprises:

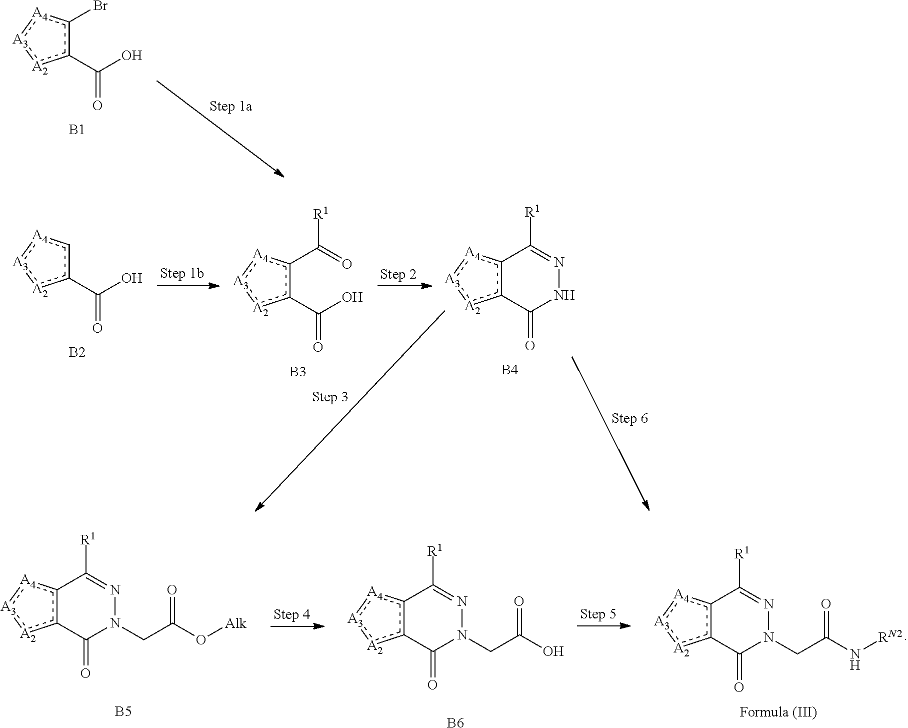

26. A pharmaceutical composition comprising the compound of any one of claims 1 to 24 and a pharmaceutically acceptable diluent or carrier.

27. A method of inhibiting inflammasome activity, comprising contacting a cell with an effective amount of the compound of any one of claims 1 to 24; optionally, the inflammasome is NLRP3 inflammasome, and the activity is in vitro or in vivo.

28. A method of treating or preventing a disease or disorder in a subject in need thereof, comprising administering to the subject the compound of any one of claims 1 to 24, or the pharmaceutical composition of claim 26.

29. The compound of any one of claims 1 to 24 or the pharmaceutical composition of claim 26, for use in inhibiting inflammasome activity; optionally, wherein the inflammasome is NLRP3 inflammasome, and the activity is in vitro or in vivo.

30. The compound of any one of claims 1 to 24 or the pharmaceutical composition of claim 26, for use in treating or preventing a disease or disorder.

31. Use of the compound of any one of claims 1 to 24 in the manufacture of a medicament for inhibiting inflammasome activity; optionally, the inflammasome is NLRP3 inflammasome, and the activity is in vitro or in vivo.

32. Use of the compound of any one of claims 1 to 24 in the manufacture of a medicament for treating or preventing a disease or disorder.

33. The method, compound for use, pharmaceutical composition, or use of any one of claims 27 to 32, wherein the disease or disorder is associated with an implicated inflammasome activity; optionally, the disease or disorder is a disease or disorder in which inflammasome activity is implicated.

34. The method, compound for use, pharmaceutical composition, or use of any one of claims 27 to 32, wherein the disease or disorder is an inflammatory disorder, an autoinflammatory disorder, an autoimmune disorder, a neurodegenerative disease, or cancer.

35. The method, compound for use, pharmaceutical composition, or use of any one of claims 27 to 34, wherein the disease or disorder is an inflammatory disorder, an autoinflammatory disorder or an autoimmune disorder; optionally, the disease or disorder is selected from cryopyrin-associated auto-inflammatory syndrome (CAPS; e.g., familial cold autoinflammatory syndrome (FCAS), Muckle-Wells syndrome (MWS), chronic infantile neurological cutaneous and articular (CINCA) syndrome/neonatal-onset multisystem inflammatory disease (NOMID)), familial Mediterranean fever (FMF), nonalcoholic fatty liver disease (NAFLD), non-alcoholic steatohepatitis (NASH), gout, rheumatoid arthritis, osteoarthritis, Crohn's disease, chronic obstructive pulmonary disease (COPD), chronic kidney disease (CKD), fibrosis, obesity, type 2 diabetes, multiple sclerosis, dermatological disease (e.g., acne) and neuroinflammation occurring in protein misfolding diseases (e.g., Prion diseases).

36. The method, compound for use, pharmaceutical composition, or use of any one of claims 27 to 34, wherein the disease or disorder is a neurodegenerative disease; optionally, the disease or disorder is Parkinson's disease or Alzheimer's disease.

37. The method, compound for use, pharmaceutical composition, or use of any one of claims 27 to 34, wherein the disease or disorder is cancer; optionally, the cancer is metastasizing cancer, brain cancer, gastrointestinal cancer, skin cancer, non-small-cell lung carcinoma, head and neck squamous cell carcinoma or colorectal adenocarcinoma.

38. The method, compound for use, pharmaceutical composition, or use of any one of claims 27 to 34, wherein the disease or disorder is an inflammatory disease.

39. The method, compound for use, pharmaceutical composition, or use of claim 38, wherein the inflammatory disease is associated with an infection.

40. The method, compound for use, pharmaceutical composition, or use of claim 39, wherein the infection is a viral infection.

41. The method, compound for use, pharmaceutical composition, or use of claim 40, wherein the viral infection is caused by a single stranded RNA virus.

42. The method, compound for use, pharmaceutical composition, or use of claim 41, wherein the single stranded RNA virus is a coronavirus.

43. The method, compound for use, pharmaceutical composition, or use of claim 42, wherein the coronavirus is Severe Acute Respiratory Syndrome Coronavirus 2 (SARS-CoV 2).

44. The method, compound for use, pharmaceutical composition, or use of claim 38, wherein the inflammatory disease is associated with an infection by SARS-CoV 2 leading to 2019 novel coronavirus disease (COVID-19).

45. The method, compound for use, pharmaceutical composition or use of claim 38, wherein the inflammatory disease comprises cytokine release syndrome (CRS).

46. The method, compound for use, pharmaceutical composition, or use of claim 45, wherein the CRS is associated with COVID-19.

47. The method, compound for use, pharmaceutical composition, or use of claim 45, wherein the CRS is associated with an adoptive cell therapy.

48. The method, compound for use, pharmaceutical composition, or use of claim 47, wherein the adoptive cell therapy comprises chimeric antigen receptor T cell (CAR-T) therapy.
